SEQUENCE LISTING (154 sequences)

SEQ ID NO: 1 - DNA, 14836 bp, Mus musculus
  source 1..14836: mol_type = genomic DNA
cctcccccgtgtctccccacacccgggttggggttgttttggttgaccagagtggaacacaacgatctattggcagggctgaacaccaatgggtctatttgtaaagcgccaatgaccactttctgaagcagggttttagggagcggggccttagggaactctttggtcctttttagaacactggactttcttctggaaaggcaggaaacactgaagtttaagaagttgtttccagcttccattaactgaacacacattaaaaccaagcacagagaatcaggacgtttcgcgggagtgagacccagtcatttctcctccgtttccattctgcagggtgagagttgtaatcacccacccactattcgtaccatccacccacccccagtcgagagaataggggtacagaggggaggtggcaaagaaaattcacgatactgagtatctctgggagacctgtttggtctctttgctcggtagcgcagccctacgttagaatgcatcttcccgggaatgactgtagtgagactttggctgggaatccaagttattctaactgtagattggtccacgttgccctaagcctagcagtccactgcggcacagacaccctggacatgaggtgggtcagcttaagttcctggcacgaaagaaagggtactctggcaacttttggatgcggcgaaacagactgtttcgtctctcaggttcttatttcacggcttgtgcctttgacagccccttagtttctctatctgcaggatgggagcattaagctctacgacccagcctctttacaattcaggtccaaagagcccgcccaagttggggactgggaagatcaaaggtctcagcacccagcggagccgcggacactgagggcgccaagaagggggtgggtaggtagggaactggaagggcggctgctccgcaggggatgcgcgtcagagaccccagccacactccaggcccgccccttgatgagccccgccccgccccgcctggttttcgcctctaaagcgcccagcgctcgcctcccgctgccgcactttcactctcggtccacctcggtgtcctcttgctgtccagctctgcagcctccggcgcgccctcccgcccacgccatggacgccaaggtcgtcgccgtgctggccctggtgctggccgcgctctgcatcagtgacggtgagtgcaatccgcggccgggcccgggaaaggctcgcagctctgcgccggagctccttcgggtccgcggttcctctgcccgcgccgaagtcgcggagaaagaactcggtcggcgccgttcactacaagcgaacttggggcagtccactttgcagggcgcactcccaccgggtgccctttcccgtgtcccacgggtcgcaccgaggttttgtgctctgcgaagtgcggccataggacctagagagggctgcaggggaggacccgcaggattgttgggcaagagtgggttcggcgcggaatggaagcgtgggcgattgtgtccggggcttgggccccggagcgcgccagctgcactcagctagtgtctaccggcgcccagatgtttccagaggcgaagggcagcgcggtcccggagttgaccgtgcaagaggttcactcgggtggtgcgtgtgtcagcaaactctcaaagaccggtcaagtagctcgaagtgcatggcttggctataggttcagtggtgaggctgagtttcgtcccctgcgggtgtagcgtgttctcttacagcaccctcgaggggctcagggccaccagcagcgcagcgcagctcttgaactcgcgctgccagccagggccgcgcttctgcacagttcgttggtccgtagcgacgcggacctgagcacgcgtctcttcactgcccctttttcttcttacccgggtcactagacaaaggctcagcagttacccaagctatatgcacacctctccccaacccccaaacacacctgcaaacgggcgcttttgtagccagccccggagtcctcagctctggaatgagagctgcagcggagttcagtctcccagacccagggtggtgtcttctttcactgggaaagggctttcattttgttttctttttttgacactgaagagaaaactctcagcgctgttacaagacaccgttgctgcaaaacaaaacaaaccattgcctctgaacacaaaacaaaatcctactagtcgatcccctgccttcctccgcagtggtgtttcctggagagaactgagggacagtcggggctcttggtgagactgagctctaaatgctgcccaagtacaccaactcgttcgtttgggttctttccctgtgacaacggggtacgggaatggttggagttgcctagtccgagggaaatgttctgtaaaagaatagtcagttgctgatcggagtagtaaaaaaaaagaaatgaaaggcagtttcgatttttttttttttttttttttttttttttgttaccgagaacacccgggaggctgagccttcccactggtcccccagtgccccgtcatggagcacattgatttgggcattaataattgaatgagctggtgatgttgcaagggtcacagcctctggcaagttaggtatggggcaagaatgtaggactcaggtcctcaaggttggagtgcaattatccagagtaaaagttgtctcaccctcaacatattctgaccctaggaagagtcggattgttgacagtgtctggatcagacctgttctctaggcaggaccccattgtgctgcccgaatgaacttttttacctcctagtgcctgtgtgccctctgatcttacacagccctcaagttgcagcacggctaaccttgctgtggttcctgtcttttcccatcagctactccaactcagaagctagatagtagacaccggaggcttctttggttaaacccagagcagcaggcttgccaggcttgttagattgaatggacccctggttccctaagccaagctctctagattcccaagtccagggtggcagcagagctggattagactttggtctgtacctgaagtctggttttcctatgctttagagtctaaagacactacccttcctggggcatgcatcccttagctaaataatgcttgcagaagaagataatcccatcatatatttaattcggtccacttctccagctgcttcccaaaggcagtgaacttcagaatacccagaagtctcctggaactctaaataagcaaacttaaaatcctggggctaactattctcagtcatacttttaaactttggtgaaaagacccataaattgaaacatttggggatgctcagtagagctaggataaaaccctgttgttgggggagcagctacaaatccagcagtcctcagggtttgcaattctagacttaaagggtggttcttaagggggggttctaaaggagccccttgctaatttacactaatgagtgtcaattatagcattttgcaaattggtgaattggcaaacaaagctggtaataggatccaggaggcctaggcatccaggtagtgaccataaaagccacggttgaccccagcttttgggaaaagctggatagaaggtaaatccgggtcctcccctctggattcttttgtgatttccagggcttaggatagggtgagtgggaggagggaaaactgcaggtggtagaagtgaagccccccacctccaggcctgcaccagagggccacaagggagcccagaactctgccaccccacttctcctgggtccttttgtcctttagaggctgagcccagtcagatctcactgtgatccctggccgaggggatggtctttgcaagaaactttctgtaaccattcctgctgatgttcctgagtcttccccacaagagccaccaaaccccctgcaccaggcagataatgactggccccacttttctctctacacctcctctaggtaaaccagtcagcctgagctaccgatgcccctgccggttcttcgagagccacatcgccagagccaacgtcaagcatctgaaaatcctcaacactccaaactgtgcccttcagattgtgtaagtcctagccgccatcccccaaagaggagcatggtatagaagcctcggacttggcataactaggggcagctgttaccaccaccaccacggggacactgatatgccatcagacatgggtttcaaaggatacttttgttccccagagccctgatgtcctcagtgtttctcactcttgctttccaagctgtttcttgcagcacagtgggccgcctctctacagaaaaagccatggacttgatggaggtcagccctcagctgacagttgggtctgtcttgtcagtttcaaggttctggtgtccaaagttaatcctttctcacatagaaaaaaaaattacaagacccggatggcacggggggggggggggttcagttttactcacttgcactcacttgctcagaggtcatttttgttttagagttttagagtttgctggagtgtgatggtagctgccagtatttgatttaaatttacctgggaaataagaaaagcccaaaaaaggtataaatgatgtgaatatctcactcagagtctggtagacttggcagagatgtgtcctgtgctagtctgtcctgctcactgccccccagcaggggttcccatcctcgggagactcaacactaacaacagtataaggatgcagcagctggagcaatgctagcctgacggctttgtcacccaacggtgactgcttcagactttctgtgctcatcagccttcctctccagcctccgctgctgtgttatgtacagtaggctttagagacctagatgatgaatattatttttgctgttttgattaaaatacaatactctcccgagaaagggattttaaagatgatgagtttacgtttgaataggctgtgctggtgcactgtcccgggaagggcccttgaacttagagggtcaaatacaactattgattctgggtgatcactaagttaataaatggcaggatccagactgacacccctgatccctgttgaagttacatccctctgaacgactggtcaactgcagggcagcctgcttgaagagggttacctgtccctaggacactgaacaggcatttgtttttcctagaagacagttcaccagctggagaggagtcgtctcccgtagtttctgtttggttgcttttggtttttgtttggttttggttttttaattatctggcatccaggacttgatggaaaataaccagagctaagctcaccggttcatctgcccattaggaagttctagggatgggagaaagaacacggcgtcaattaacaaatccacaaagctaagaccttgaagcattctgtgaacttgtaaacgcgctcaggcaaccattggacaatttgtctagactgctccttgcccacctgaactgccctgttcctccccttctggactcctgccgtcttcctccagagctacctttaaggttgtcccatgtactatcaaggtgctctgtcaaaagttcttaggctgcttctggcactctccagaattttccaagacctcccccccaccatgatatcagtcatccgcgccttctgggtggttcttcctccacaccctttgggcactttgactcctgtgggatattcgtccttccttttcctttagctttcctcacttgccaagctccaacttggccagaagctcaaatgcctccactgtggtctcttctctgtgtcccctgggagacatccttagcacgtccctaactctgcggtggtggtcccaacacgattcaagtgctatgtcttccaaaactgaagcttccgggagcagcagctgggccctgcagtgaggacctttagctgggtgtgttgggtgagcccacaggatcgctttctcccgcttggctgtacagcgtctctccccttgtgttttggcagtgcacggctgaagaacaacaacagacaagtgtgcattgacccgaaattaaagtggatccaagagtacctggagaaagctttaaacaagtaagcacaacagcccaaaggactttccagtagacccccgaggaaggctgacatccgtgggagatgcaagggcagtggtggggaggagggcctgaaccctggccaggatggccggcgggacagcactgactggggtcatgctaaggtttgccagcataaagacactccgccatagcatatggtacgatattgcagcttatattcatccctgccctcgcccgtgcacaatggagcttttataactggggtttttctaaggaattgtattaccctaaccagttagcttcatccccattctcctcatcctcatcttcattttaaaaagcagtgattacttcaagggctgtattcagtttgctttggagcttctctttgccctggggcctctgggcacagttatagacggtggctttgcagggagccctagagagaaaccttccaccagagcagagtccgaggaacgctgcagggcttgtcctgcagggggcgctcctcgacagatgccttgtcctgagtcaacacaagatccggcagagggaggctcctttatccagttcagtgccagggtcgggaagcttcctttagaagtgatccctgaagctgtgctcagagaccctttcctagccgttcctgctctctgcttgcctccaaacgcatgcttcatctgacttccgcttctcacctctgtagcctgacggaccaatgctgcaatggaagggaggagagtgatgtggggtgccccctccctctcttccctttgctttcctctcacttgggccctttgtgagatttttctttggcctcctgtagaatggagccagaccatcctggataatgtgagaacatgcctagatttacccacaaaacacaagtctgagaattaatcataaacggaagtttaaatgaggatttggactttggtaattgtccctgagtcctatatatttcaacagtggctctatgggctctgatcgaatatcagtgatgaaaataataataataataataataacgaataagccagaatcttgccatgaagccacagtggggattctgggttccaatcagaaatggagacaagataaaacttgcatacattcttatgatcacagacggccctggtggtttttggtaactatttacaaggcatttttttacatatatttttgtgcactttttatgtttctttggaagacaaatgtatttcagaatatatttgtagtcaattcatatatttgaagtggagccatagtaatgccagtagatatctctatgatcttgagctactggcaacttgtaaagaaatatatatgacatataaatgtattgtagctttccggtgtcagccacggtgtatttttccacttggaatgaaattgtatcaactgtgacattatatgcactagcaataaaatgctaattgtttcatgctgtaaacctcctaccgtatgtgggaatttatttacctgaaataaaatctactagttgttagatggagtgcacatacatttctgaagatggagaaaaacaggtgtgcctgctgatcaggtgctgtgggctgccctgcagtcctggtgagcgacagacactgaggcaggcttgtctcatgaacaggctgcctctgcagtgaaagtttttgtgtattttttttaacccaagctagttttctaatgaataatacttgactcactaatttcccctcctcctccttctcctcagttctcctaacatcctcatgtgatccccagactcaactccagtaatatcaagctttcctattttcccatgtaaaaaaatcccatgactctgggccatgttaatatcaggcttttgtgggaacaggtggcctcaccccataaatcattaaataccattcagcttgaatcattttaatgtgacagtcacaaaccagttgctctaataaaaactctgctaaccatccttctccttagctctctagaacaatctcagttatccctagggatgctccccagcatccagaaaagagaagtgggatcaatcatcctgcctttctccccctcctctcttggagggctgcctgagcccgtggcctccacctcccctgctttgtataatttgaaatgcagatttgtagtgaaggcagagttcacctctgcattgaaagggaaggcaggcccagagcttccttccctgccctctgagatgtgcatttatgtctcaggatggatgagctttggtaggaatgctcaaaaccaggaccagccagacaaactggcagtccctgtaagcggttcccgggtcatagggttagggcacccctgtttaactttggggtggggaaagtatctggttttctttgataaattgcttgtgaaccacatttgccaagtggcctccaggcctcaaactcaaagaccgagctaaatcgactcggaaggcaatgctgaatgaagattgtgggaactgagatagatacactcctctatgttgcaatgtgattaatggttctactaattttatctaagggggcgcagagaagaaaaagtggggaaaaaagaaaagataggaaaaaagaagcgacagaagaagagaaaggctgcccagaaaaggaaaaactagttccccgcttcctgccgatggaccgcagtgcgctctgctctggcgctttgtaactcgctcctccctcttcgggggcagaccccacactccgggcaggtgctcaaacctgacggtaaactcttccctcttcgggggcagaccccataccccggggcgggtgcttaggctttcctgccctggtggccacaccagctgctgtatttatgtgcttcataaggccctgctctgtctgctaaagctatgaagaaagatgtgcagagactggggtggagactaagccaaagaggagctgcctagcctggcagcattgccccgagctgagcccccttggccaggacttcacaaggctcacacctacaatcccatgaaggccagggtggtctgcttagccaggaaagggcaagtgccttcccctcggccacactgccccttgtggccttctcgggacatgtggtaactgacttgctctcaggcccacccgcagcttttccaaatacctgcagccttcagccctgctgccctgcctgtgggagcagctttgactccagtccagaagggtttctgcagactgtgttgggtgagacgcagaaaggatgaaatctcagaacacatgtcagctgcttctcaggaaatcttttctttggacaattcactttagagtctttaaacgggtctctcgtggggaggatagatgtgctctggaactttctgaaggaccagcagcttcagggactcttagtctgtccttccccacttttggtcccaacatccctgggatggtgtgctgtctgggcaccacggtctccatcctcactcctgagagatttctgccttctgtgagttgggttaaagctctggaattatctactatcccaatccactaccctcacctggcaatatttgtctgtttttgtttgtttgtttgtttgtttttgtcttttgccagtttgaattagaaggcaaggctctgattttagtagtgttttggaaaaggacttttttcttcaccttcctctttgcctcatgtgtacacacacacacacatcttgtaccccagacctctgggtataattttcataattggtgcagaaagaagaaatgatctgaagatgtgttaaatggattgcaggggaaggaaggcccagggccctgtgtgtcatgccctcttgggttcctaagttctatgttccttagaggttctagcattaaacagataaagcccttcatggtcctggctgaggaagagtcttgctagggggattcagggaagacccgtgttaccagctcttaccctttatctggacagctctcctaccctgtatcttctcctcagatctgaggatagcaggctggactattggtgggcacctttcaagcccagggctactgtttgtcctgtggcagccggctacagtctcgtctgagtggcctcatctggacccttcctgttattaataaaacgcttctggaggccagatctgtgctcaagccatagttctgcttagaaagggatgccccacccttaccggacactgggaagaactgttggcccctagaaaccaaaggccaaactgaggctgccctgagttggaagaccactttctgaaatgcccatggactctgcctcccaaccattcgtctctcactcctagcagagctgtctgtgcagactgtttcttaggaggcacagcaagctccagggaaccctctgtgcttatgaagctcgtctggtgggcaaccccagcccactggacagagtcctcatggaaatgcctgggaagctgatttcatctaaggatgggttgaagtaggatgtgctcctgcgacttctcaggcaggtgagaggggtagtccttacactgtctagcataaacgccttccggaaggacctgcagctccagagaccacctcctgagcaccaagacctcttctggtggtgtggaaccagccaagagatttcaaggaagagtgattatttgatgaatgctatgggaatggcctcttctcttggagttctgaggcctggggatgcccaggaacactgggcacctgctgctgttagggccaatgcatagtctcagcaccggtgtcctaaggttaaggcggtgcgccttgtcatgtgctccttgtaccatgccatctgtgccagtgtgtgtctgcctcaccctgtgcttgacatgttcacccatcttctctgcttcccgccaccatccagatcctcagcggccgccccggctgtgcccttccctgctctcccgctctctcaggcctcggaaggaagatcggtggctgcgagctgaactaaggagtagggcctgtggctcagcgctaggccacgcacgcagcatcccaggcatgtggtgagaaactgccttaatgtgtctcctctgttcttgtcaacaggaggctcaagatgtgagaggtgtgagtcagacgcccgaggaacttacaggaggagcctaggtctgaagtcagtgttagggaagggcccatagccacttcctctgctcctgagcagggctgaagccgtttgcaagggacttgctttgcacagttttgctgtactttcacattttattatgtagcaagatacatggtgatttttttttttttcatttagcctgattttccaacgtcattggtgacaggccaaggccactatgttatttcctttgttctggtatccttcccttggaggaccttctctgagtagtggctccccaggtttgtcctttgagctgaggcaggaggctcacccattcttctgaataggaactgggtgttcccaccccccaaggactgcagggctttcccaagctgaggcaggaacgtgaggccagggaagagtgagcttcaccctcatcccacgctgtcctcctcaacccaccatgctcatcattctgtctcatccatccatccatccatccattcatcgccatgtgtccgcaagactgtctccatgaccctgaaaaaggactctcgagatgaaatcctttattcaaatgggacagcaagaaggaaaagccaatgtctggtgtctctccccccgcccctaccctgcgcgcatctatgtcttgtttggaatattgtctcttcaaccccctgttcatgtccttctcactcatgatcgatgtcttgtctgtgcactgtctctaacccaaatgcaaaggctgagtgtgaggtgatggccccgaggtccaggttgtagtcatggaaagagccctgctgtctcccttctcagggggcccattttagacacacaaagcccaaagaaaggtggtttgcaacagtgcttagctcgagcctccatatttccataactgttagcttaaaactgtggggttttaccttcctggaaccaaatgcattcttctgttgaggagtaacaggtctcaattcttttcaattaattttaaaagtcaatcactaagagcatcggctttgggccctgatgggcaggcatttccctggaaagggggtgaactacctacctctcctcaagacagccgaagggtgggattggtgccgctctgggaagcgtggccccaggagttttgtcctctgcagtttttaatgcaagttcactgccactttgacaaaagcccaattagaagccagtctctagttccttaaacaaaacagacagagtaaggaaaggaaggagggtggcagccagctggctggacactcgagaaagacggggaagtaagctacagaaagatagtcttcaaaaacaggtgtttgagagtgaatactctgtagaattgttagtggggtgtgtgtggtggtggtggggggatttctacaaaatagtcctttaagttgagtttacagcagatgaaaaatccaaccagcaaaattttgatcaaatttgaacaaaaacccaaaaacctaaaactgttgagcaggttgcgatgaggagcacagggctagctgcagagctggatcctcaggaggatagcgaattattttcaaccctggaatagaaaccacacactggcttgctgtgcaccagccactttgcatctaatccaagctttgaagggtgttgcttgggaggaaacaaatacagccttccatcttcactccagttagggatcctttcaaagtctccttcacagtgaggaaaaagagaagggtagaaactttagggagccggatttgtgtatcaattcctccgctgacagtcagtttctagatggagacagcctgcttaaagcaaatccgaatttaaataggacatttacatcggaaaagtctctccctaccttaatcccccattctcttgctttcaaaatacaagcacagcagtccttgaatggctgttgacccagggcacctagctgtccctgctggtcctggggctgccagaattcccttgggcgccaagcaacctgccaggtagccagtccctctgttacaagcctttgcatctggatagggaaaggggtggagacatacagtctgctttgtgttgaaacccagatttgtaccctgtgtttatacactgctgctggctcccgaggacagtgggactttagcaaggaagtgcagccgaggggtaaagagccctctggttcattgcctgatcggctttgagagagggtttggagggcaaggggctgcattcctctgagggacttggcctgaggcctttcgggcctctccagtgggttctgtttatcctctcatgggtgattatctcagtggtgtcaccaggggcttcctcccagaagtcagtcatccccaggccgtgcacccttttcagctggatgagagccagggatgcattctctccaaacagctaccctggcccattttaaggtaatctcattcttcaaaatgttccatagaatcctccaaattcccccagcagacttctaccctcgccaagttcccaaaacccactcagcaaagttgccaacctcgacgggctagcagtgtctaagcagcgatgggttcagtgttgtgtgtggtgaatactgtattttgtttcagttctgtctcccagataatgtgaaaacggtccaggagaaggcagcttcctatatgcagcgtgtgctttcttattcttatttttaatatatgacagttatttgagaagccatttctactttgaagtcattatcgatgaaagtgatgtatcttcacctaccattttcctaataaagttctgtattcaaatatagctgccaagcatcctcagtgaatgttaccatgtggaattttccacacttggttttaccccctcaaacctgactctgaccgtgcagtcttagcagaagagcttagcaggtcctagtgttcactcttggtctaactgctggtgtcagaagatctctacagggagaggtgttccattttctccacatgacctggattgctccttagaggtcagacagccttgcactgtacaaggcaatggcttagggtaaagtcccaggagttttccctacagtcccaagaatttggaagaggaaggcccacactacacatgcaggtcatggtggaaggtgacagaggaaggactctgtccctgtaagacagctggaaaccacaatattctgcatgttcctatcctgggtgaggacgctaatggaagtcaaaggggaatttgctaactgctgttggccagcttcctccaagaatcctgcttccccaacagacagagcctttgtctcttatagtttggtcttcagattctctttatcccacattcagccatttttgtaaaagagaggctagcaccagctccaaatatccaaatctgcagtgtttgagatctcactgcgcctcctccataccaacacatttgccattacttatagggtagttttcatgtgagttctaagttgattaacacacaagaattagaagggtgggaggctctaggaaaggcactgtgggactatttgactgcatgggtgtgaaaatgtaaggaacaggcaagagcttggatcccattctctctgcccacattgtgacttgagatatactaattgctcttgggggtctcagtcatataccatccataacagagttaaactgagagagatacaggatcagctagaatgaaaagcccaccccatgcttccagaaagtcccctctttatacctcctgtgatatgaactagaggaaaagcaattgactttgcttctcaaacagcctacggcaaagccctgtgagtttg

SEQ ID NO: 2 - DNA, 25001 bp, Mus musculus
  source 1..25001: mol_type = genomic DNA
aagggtttctttggcttacacttttcttattgttgttcattattgaaggaagtcaggacaggaattcaaacaagtcaggatcaaggaggcaggagctgatgcagaggccatggagggatgttacttactagcttacaccccccccccccccccggcttgctcagctttctttttttttttttttttttttttttttttagatattttctaatttacatttcaaatgctaccctgaaagtcccctataccatccccccacactgctccccaacccacccactcctgcttcctggccctggcattcctctgtattgttgctcagctttcttatagaacccaggaccaccagcccaggaatgacaccacccatagtaggctgtgccttcctccactgatcaccaacggagaaatatgggcaacagcttgcccataggatgggtcactagttgggttggttactgttgggccattccctcagtctctgctccatcccccatctctacatttcttgtagacagaatcaattttgggtcaaaagttttgcaggcaggttgttgtccctcttacatttctcatggaggcatttcctcaacttatttcctctctctgatgtctccagctcatgtcaagttgacacagttgttcgggacccacatgaagaccaagctgttcatctgctacatgtgtggggaggcctaggtccaacccgtgtatgctgtggtagttcagtctctgagagccaaaacagtccaggttagttgactctattgatcttcctttggagttccatcccctttggggccctcaatccttcccacaactctaccatcagagtccccaagcttcatccactgtttggctgtgggtctctgcatccgtctgagtcagctgctgggtggagcctctcagaggacaggctttctagactcctgtctgcaagcatagcattaatagcatcagggattggtgcttgcccataggatggatcactagttgggttggttactgttggccattccctcagtctctgctccatcccccatctctacatttcttatagacagaatcaattttgggtcaaaagttttgcaggcaggttgttgtccctcttgctccactgaggttcctgcctagctacatgaggtagcctctttaggtttgatatcctcaatgctgtgaatcccaactaagatcaccccccattcattcctggtgtctcccctatctcaggtctcagatatgccttcaagatgccccccccccacctctccacctctgccagctgcagatttccattcattctcatggccatctggctatctctcctgttcctccccatacctggtcctgaacccccttcaccccactccccatcccctctcccacccagttcctttcctccatcttcctcctatgactcttttattccctcttctaaataagattcaagcatcctcgcttggacattccttcttatttagcttctttgggtctgtggagtggagcgtgagtattccaacttctaaggcacacagacaacctcagattctccagccctttgtgtgtgttgcttatttgaacaaacgggtgaaagaaaacacacaaagttggcgtgttgaaagagttagtcgatcttctggggtaggtttcagtacagagaccaaagggacattctcagacactagacacactatgcaaagacaggatgtcacatgacaaaggataacggcacaagtaaacatttaagcaacagtgttccataccggctcacgtagaaaaaggacaagactataggaaagaaagcaaacactccgccgaggactacagcaaagacagaaagtatctgcaggtacggcttcaaaaggagcatttctctcagcaacttatatctgttaatgccctgtcttctggaataagggcttagtttttatcagtagagagagattgatttttaagatgtatctgattttacattgtagatctccttagtcaccccctgtagtaaactaaggaaaacttccgtggagggagaggggaagattagtaactcgtagtgagtaagaattctctttcaagaaaaagattcaagagcaatacaaggcctagatatgaaggtgtgtgtgtgtgtgtgtgtgtgtgtgtgtgtgtgtcttaacagcctgttcagaatttagtaggtcacatacacttacaagtaatgaagacaatatattaatgaatttgcagtaattttttgttttagaaatagaaactgttgtaaggaggataatcattcagagctctttgatatgtatcactcacattcacatacatgcatacacacagagagagagaaagagagatacagagatagacagagagagaacccaaaaatgtaaagagaggaaatgagttgaaagaaaaaatgggaaactgggttagggaggggttcagatgacagtgactgggggctttcagagttgggagtgaggcagcgatggagagagggcagggaggagggagtgtccattgtgacctctgcagaactctgactagactgagcagctcacaccgtgttggagctgtcctaacactaccaaggggacgggggagaccccatgaacaccacctagggagttgctcctttcattctgtgtaaagtctgatgtcttcaaacttgttgtaaatttatactctgttctaaaaacagtgacattcttctctttgtaggatgacctcattacatcaggtgttatattttatcttttttgcctcagtttctagtggtaagttgctgtatttattttcccctaacataatattttttattacttgagagtttcatacaatgcaccctgatcacactcactttccattccttctaagttcaccctcccactcttgagccctgccagctcactccccccttcttgaaaaaaaatcatcaagtcaatcaatttgtgttgagaatatatactcgttgggaatgtgatcaaactcccaatggtcagccccttaaagaaaagtgagtctttttcctcccccacttctctccccactcccttacccagttggaagccatcaactgtgaagagttatacttcagcatctttactacaattttaaaggactctcttcagtatttaagtatggcttagaaatagctcattccttgacctgtaatgtaggaaacagcctaagtccacaaaaagaaattacacttcagaccccatatattgtggaataattccatgctgtgaactccagggaaggaaatagagtcgtttattttccagtgaaagctcccctttaatacatcaaagaaagaaagagatttaaatatagaattacaaagagtcttcacctatatcatctgaatgctagtaatatctgtctatagagttgcatctctatctacctacacaacacattgccatgattcctaggagcaagattagaaagagaagactcgactcacctcattgattattattccataagggattcagtctagtatctctctctgtctctgtctctctgtctctgtctctgtctctctctgtctccgtctctctctctctctctttctctctctctctctctctctcacgcacacacacacacacacacacacacacacacacactcaccaattcctgactgaaaatgttatagaaaaattaatgtgtggcttacacatttggttaatttaccccttgcaattatgcttccattctacattacatccagtaaatacattgcttaccattcagtagaatgaaatgggaagttacctcaccaatactgatcttaacaacttagtgtaagcacttcttaaaataatttatttatgttatattgaatgcctgagactgccattgacatattaagcatagttagttctttttggtgtgacacatgtgaacagtagcagatctaaaataaaataaacatatgtaacatattaaattatacagattatagcttaatttttctttgtgattagattgattttcaggttattccttcattatcaatgttttgaaatcccattgttatttgtactgtcttgttcagtactgttttgacatgttgttgttgttgttgttgttgttgttgtttgacacagagtttctatgtgtagccctggctgtcctggagcttgatttgtagaccaagctgacctcaaactcagagatctgcctgcctctacctccaagggctgggattgaaggtgtgcaccatcatcgctcggcagcctgtcttaacatcttaaacactgagttcaataactgtgtcgattcacaaggacattctgagaattataagacttttttgcttatgaatatatatatgcaaatgtaactgacaaaatattatccattgtggttgtatcacacttaaaaatctcagagccgagaaagttggggcaagatgattaaaagttcgaggacaggatgggctacataacaaggttctgtctcaaattggctataccaaaccgtccaacacatattttaaagaaaaataaatgggaggctagagagatggctcagtagttaagagcacttagtgctcttgcatgggatcagttcaattctcagcgcccatgttagatagttcacaacttcctatgactctaacttccaggaatacagcaccctcttctggcttctgtaggtacacacacacacacacacacacagacggcatacgtacatacatacatacatacatacatacatacatacatgcctacatacacatgtacatgcatacacaataaaaaagtttttaaaatctttttttttaaagaaagaaaattaaaagaccaattacattggcatattttggccaagtttgcttaattctaggaacaaggagttactttaatctaagaaaaacaatcaatggatgcaatgtagatccaaaggaagtgaaagaagagagtcctacagagatttgtcatttgtcttcctgctatcgggcagagaaccagcaagagagaaacgtgggcatttgaagcccactcagccgtgtcatagcacaagttgggtcttcaccaatggacagaaggttaaacaaaatataatatcacagtatgcacatgcaacacaaaacaggatattactcagcgtgtgaggaagaaaattctccctcatactggggcatagctgagccttcgtggttttatactcaacgaaatgagtcatttacaaatgaacacatgactgaacccctaacgtttggttcccagagatgccgatttcaggaaaacaaaagaccgaagagaactgaccaggggctaatgctaatgactatttaatgggtacaagttttcagttggaaaagctgaaggaattctagaatagtggtggaaattgtacctaaggtacatacttcattccacaactctagagacctgaaaagggccggagtgacaaactttatgtcatatatattttgccatagaagaacaaaattaaaataatctaacacatcgaagattttaaagattttcataataaaatagtttagcaaactcagaactttcccaatgactaagtagtactgtaaaacaacaacaactagaaaaacattcaaaccaaaagttttcaagaaatctcatatgtaatggtgactgaaatagtgtttccctgaggtactggggtaaggcaggagtatccacaacttggctcctgtgaataggaaacaaggaacagagagatgcaagcatcttcaaaaagttgtcataggatccatcatggacattccaggaggggttcagttctacttttagttttctgtgacatctcattacaggttttgattttttccccccatagcttcgccagcatggaaatttattcacaagtaccaagtcagggagtctgcctatgtgtccatcaatagatgaatgcaaagagcaaacactggatgtaggcacagtgcagggggcagggaggggtggcatgttctgcatttagctaaaatggttgttttagtttttactaattcttcaagaatgccatataccatattttgctaatttttacctcgactccccaaactccatccagaaccccccattctgtacccacctaactctgtgttctccctgtttcatttggccacagtgaggaatgaaatcgtgccatttgtgtaacaatggctgtgactgggcatcatcattattcagtgagataagtcagattgaagaagataaatatcccattttctcaaatttgtggattctagaccttatatggatacataaaatccagtgtgtgtgtgtgtgtgtgtgtgtgtgtgtgtgtgtgtgtgtgtgtgcttgagtgacacactctaggctccagagtctaggggaatgacagagactagaaggaaagagggaaacgaaaagatatgaagaataaatatagtcgaagcacatgatacatgcctatgatattatgatgtatacacacctatgcaggtgtcccatgaagccccctgctatgcaacgaatacgtgtcaataaatgtgttgagagactatctactgtcacttcccttaaacattatattagagatcctgtgtagcacagtagggaaggtcgaaggacctaagagtcaggagaaagatggaaattaactgactcttcccataaatgtggttatgtacatagaaaaattcaaagtaatgaatgcactgattatttcaggcagatcagcaaggagtttacaaactcgatggacccccgggagtggaggcacacacttgtaatctcatgactcaggaggcagagggcacatcagagaccctaactccagctcttccgttcaaaatcttcaatatacaaacagtagccaactctcgccagtactaggaactgtactgagattatgaaagtcaatattataggttaaatgattcaagtagtggctacatcctaatcaaaactgcacatttattaatcctttaaaaatcaattcaacttatcaaatcctggtgttgtgcttgataagaacattttaatgtgttatgatggcttaatagtaaaatgttgcatttcttagagttaacaggtaaactgtgaggtgggaatagactgccaattagctggaaacagacgcagtatggtttggtgggagacaacactgaacttggaaaaagaagtcggatctctatgtagctcctgggatacttgcctgtagtggagaaaaccatgttccctaccaagtctctgctgaattttggaaatggatctgtagtgaggttgctcttcctccccacaaatcccttccgaggccccatggacgcagcggagcctatccttggaataacttttctaaaggcatggactaaaaatatatagggttgctggggaaattaattatattgaaatatgagtagctaaagtggaaaatactcagtacagaattagttgtggctcggagttaacaccttacatgacattagtggcatgcccaaaactgaatagaaataaccaacaaattatcgacaaatcataatgatgttatatctcatcatcacatacatgacaaatactaatcatgttaattgattacattaataatacaggtatgtatgtaccacattttggttagggaatcaatgaaaaaccacatgaggtttttttgagctcgttggttatctaggtcctacaagcctcataggagggttttgagactgctatgcacctacaatcctctcaagacatgactgatggctgttacagtagtctatatttgagttaaattttattcttctggaagtacttccacgtgagtctggtaatgttttcaagttataaagagaaatgggctagcaacacctctccatgggcatgtatggctgcatgaagtaatcttttattcagtcactaggcaacctactgtgtgaattattagctatctcttctagcaacagagcacttttgaatttttatgcattttggctttttaaaaactaaagctgatgtaaggttttcgctaaatagaactcagtaagttttccctggtaaatgttggtcatgagaccatttttaatgttcgcatgacatcatacaagggcccctcagtcttggcttttagtttgtggtagtgtcaaaacaataaatgaacgtaaacacagcacacccttgctgaccccctagaggggtttcatggggaccgtgttcttcttgccggtcaacagctgagaaatcactgagttgctcctgcagccttcccaagacaatcactcataaatgtgacaacacggagggcccccatgttgtttgtgatttatagttccatctggggctgttcactgaagagcgttgcccgatctgatgtccgaggtcaggcatatttagcactgactttttaacaaaccatgtaacgaggtaagtgcccaccaacatctgcagtaatgtgcgcccgcgtgtttttttcttcacagaatgcgttactaaggtcttcaaagacatcagctttcaaggaggtgacctgagtactgttttcacaccgagcgccacatactgccgcttggtctgcactcaccacccacggtgcttgctcttcacgttcatggctgagtcatcttcggatgatcctaccaaatggtaacagtttctgtttctctgaagaggaactgatttccagtgccagttactcaccaatgcagattaccttacaaccccacttttgtcattttacaaaaggggatgttagtggctggaaagtgagtttccttccacggaaatactaaatattacaggaaggatagcgcttaggcatattggtggaaaaccgtaataactcctgggacctcaatcttaaagtcacgtttttgctcttcatccatggtttgtcttacattctttttctgtcgtggctattggctaacaatggtggggttatctagagcttccttctaacctttcaattagggggaaaatgtttaaaaagctactttaaaaattattacaacaattttctaccccctccaatatcctgtcatattatgtggctttctcggctcccatcagtggttcaataaggcatgcagacttttatataaaacttaaggcccttcattggggcaggcagactctcagctctcttctctaatcttagatcgtctgccaagtagctaatgatttatctcgtttccattcctttccctatctgcctctgaatctcccacctctgctttagttctctctctctctctctctctctctctctctctctctctctctctccctctctctctctctctctctctctctctctctctctctccccctctctctgtcccacccccagaagtcccacccttgtacttcccgtcccactttaggcaatatgagtgggtgaggaaggacaagcacttacaaatcagaagctggtgatgggccatagacatgacgataccaaacatctgccagaacttagctctttgccagtacagtaatcaacaattgcacaattgaaagtacagagacacgccttaatacaatataaggaaggtcatcgcaacactgtcatatttgctttacctacctgtatatggtcacagataaatactgaaagtgagtttaaaatatcatcgacctgcatatctaaatacttcaacatgaatttcccaataaggaaactgtaagaagcccaacaaagttaaacatacttgaaatgttaattttagctgttaattaaattctcatcaaatttcacaggttcctatggctgtttttccctctctatcttatttttaaaatattgcatccatttctgtctctatgctaagcttgaaattttaatatataacctttaatgcaaaattaagctgccaatatctttgttattgctgttttctcagatggttacatattgctgtcaacattaaatattcttctgaaatctagaacagagatcggcaagacaggaataaaactttaggctttgttgaaccaaatgcaaaaagaacattctgtgtttctaacactgttttagaatgtaaccatgtgtgggctggagggattgctcagtggttaagtttgctggctgctcttctaaagggccctaggtttgttaccccaacttacagagatttgcaatcattggtaactccagctctagatgccatcttctgcctttctcatgcatgtgatcacacacacacacacacacacacacacacacacagagagagagagagagagagagagagagagagagagagagagacagagagagacagagacagagacagagacagagacagagacagaggcagaggcagagagagagacagagagaggcagaggcacagactgagagagagtgtgtgtttaaaatttaaaatgtagccatgtaaaaaatagaaaaccatttctatctcaaggataacacaaaaatggcagattttaagcaacattttagcaataggttaaagtccttccattcttattcatgtcctgttggaaaggagggccgtgtgtgttcttttagatgtagggatgttgtaataaaaaacaaagtcaaatcttgctaattaaacatttgttttggcaggaaacccagtcgtttgtctccttctctttgaccttaggatcccctctttgaaagcatgaattcctgctgaataatgttattttcattttattttactattttattttttaaaaaagaacaggtttgcctgcatcctgaaggacagcgtcacagaaatattgccaatggtaaacatgacaggcgcgatctctggatattccttcaagcaatgccctcagcaattaagtagtaagattttttttatcaaatacaattaaaactagccattagagtatatacgtgtaatcgtttcagatacaggtttgtggctataaaataaaatactaccacctaggactaaacttcccaccaaggaaccatcttcctatgtagagtagaggacacagttttctcccttcctctccccataagcacactgtctcgctttaccatttcttagcctcataaaggcatgtcagcacctcagttcttattattgcacacgaaagccctcgttataaaagctaattcaaatcaagaggagaaatgcagactgaagtggcacatgttttaatagtggaacgggcacttttcagtaagttaaggggtgaaccagttggtcagtgcggtttgaggtcagaaagtcaaaattcaaattcacaagctttctatttgttaaaaaaattttttatttattttatatgcattggtgttttgcctccatgtgtgtttgcgtgagcgtgtcagatcccctgaaactagagttacacacagttgtgagctgccatgtgggtgttgggaattggacccaggacctctggtagagcagccagtggtttgaatcgctgagtcccctctccagtctcaaatgtacaagcttaaagcggttccagtctaagaggcacattggcttcctcggaatacctcctagtggaagtgggaatcacagctgagtgaaacaaaacatagaacttccacgggagagtgggcgagaagcgagcagtttccacgcgagagggccagcagcgaaagcagcgtactgtagttgcccctcgcttctggtgacgcctagattctccgcctttatttccagcttgcagcaaagatgtgtacgtgaacctagacatgaagggcatgaactataacagctctgtcgtgaagaatgctcgagaatgccaggagagatgcacagacgatgcccactgccagtttttcacatacgcaacagggtattttcccagtgtggaccatcggtgagtgagcgggagtccgagccgctggatataagcctgcccagggaaagaaaaccgctggttccgtaggtattttcatcaatttgaagcctaaacttctttttttaaaccccaagatatttgcataacaacaatcactgttttgtcatgaaaaggtcatagcgtgtctaacacacatttacgacatattcaaatttcagaactggaggatggctcggtgggtgcgtaaacacactgcttatgcagggaccctgagtttggatcccagcatgcacataaaagccagatatgggtgtgtatgcctttaaccccagtgcttgggacatgggcacgggacagagggaggatggttggagctcactggttagctccaggcctttattattattattattattaaatattgtctttatttacatttcaaatgttatctcctttccctgccccccccccaaccccctctcccatccccctcccatttctactaggttgtttacccacacaccaacccactcctgcctccctgccctggcattcccctacactggggcatagagccttcacaggaccaagggactctcctcccattgatgcctgacaagaacattctctttagctccaggcttagggagagatcctgtctggaggggaaaagacagagaggtcatagggcaggaactctaacaccccccccccacttggacttcctattctcacttgcatacccacacccccacaccccacacacaccatatacaatacatacagcgtggcctcacagattctgtgtatgttgtaaagcatacacaagcttaccctatctaacttcaaaaggcattacattttcacgcttgtggttctgagagctcggtttgcttggccatgctcttcccagtctaacaaatgtcctaacctaaaaatcatcaaaacttaaagtttgtttctctatctacctacatgtacagttctgtcccttacccaagacgaagtcattggaacccatgtgcaaagttttctcctgtttgatgtgggattccaaactcctccaaggaagaaatctgttatataaactaacgagagggaatgactaaatctgcatcttcagtttaaattgttgttagaaaagtatatgacttgttcttttacaaacattttaattttgtgtgtgtgtgtgtatgtttatgtatgtgtgtgcctgtgcgtttagacacacacgtgtgcagataactatatagccagaagagacatctctcaatctctcaaggttcatttttagacttaggaatctttgacattatctagatttgcacagtaaacaacaggtatgtggtagttggattttaacattggctattcctacttcactttccataattctgggggggaaaagatgaatgggaagtgaaacattaaagatgtcttttagaatgaataataaaaaaatggaaagtagccttctatggctctctttagctctcctcagaggacatgttttatgtcttagacttcacagaatgccaactgcaggctaggaaaatgttcctagggcttacacaaaagcgtatttggagggctaacgtgaacccgttcacacaaccacctcacaccacatcaggtcttctgtgttgtcctctcactgatgagataacattattcctgagtccacagagcctccctgtggttagaagagctgccaggactcatgcagccacctggctctgcaggtgaacactcctgggcagttcctctgttattacagtcatttcccccctgcggaccgtaacttcttatcctctcgtgtttttatacacttggaaagaacaatattttgccatttttgtttgtattcagtaaaatgtgtcttttgaagtacacccgaacggggacgccaaccacaataacgaagctcaatggcgtggtatctggattttcactgaagtcctgtggactttcaaacttgggtaactatcatttttctcaatgagatattggtaccattaagcctgagtgaagcagagactatgtgcaatgggtctaactttaaaaacagctgatggttatacatgaagcgaacccagtaaccttttactgtcttcaaagtgaaatggttcactatgtcctggaaagcatttccttcttaaatttcaaatttgttctttttataaacaacaacaacaacaacaacaacagcataaataaataaataaataaataaataaataaataaataaataaataaataaggtcttcggatgaactttccatttataatacaatttacaaaccctgtctgggatgtctatgattttgctggtgcccctgccttcctaacacagtttcttcccttggacagcttgtatcagggacattttccctaacacggtgctggcagaccttaacattgacagcgtggtggccccagatgcttttgtctgtcgtcgcatctgcacgcatcaccccacttgtttgttcttcacattcttttcccaagcatggccgaaagaatctcagaggtaaggcgttgtcattaagggtcatctggtctttttaaaaaaacggccaataaaaatgtgctgcacaatcaagataggaaacgtctaggcagcaggacacttctggactccttgagatagatttgaattgcggaaaggaatggtaccagcaggaggaaagactgggaccacggacaatagggcaaggttcaaaagtgttttgaaaagttcttagtgacattacaatttacagaacgcgacttggtgattcaagaagcaatgttaggatgaggttgctattaaatgcttctctgagctacctttatttgctatacttgtaccaagtggtctttctctttgctatattttatctgatttattcatacactccctttggtcctttagacatctttgtctccttaaaacctctgaaagtggattaccaagcacacgcattacaaagagccacgccctttcgggcttcagtctccagcactgcaggcacagtgtcccaggtaaacaatgcaggctgtccctctctctgagctccacagccccaaggaactggatggctgtgaaggctacacacttcaaacctggcgtgtgctttgttgtctagtattctgccatccgtccttttacaacgacactgatttcttgggagaagagctggacatcgtcgatgtgaaaggccaagaaacctgtcagaaaacgtgtaccaataacgcccgctgccagttctttacctactatccatcgcacagactgtgcaatgagaggaagtaaggcacaagttaggtggatgctcttggagcatctccttgtaggatgagttttgcttacagagttttgttttcagccgcaggggcagatgttacctaaagctttcctccaatggatctccaacgagaatacttcatgggaggggaggcatctctggatactcactgaggctgtgcaaaatggataatggtgaatacttgaaaaaatacaactgaaggggaatagtcaacctaacgttgctagtctactacacgaggctagtctacaacaaccatagagagatggagacagcagcacaaggaggttgaggcaggagaatcagaaatttaataccagattggattaaaaggcaaaatcctgtataaaaaatgacaacaaaatagacatggaagagagaacaaagttaacaaatttggaggttttcccttacatatatgtatgtcatatatatatatatacttatatatatatgtatatgtgtatgaatatgtatatgtatatatgtcatttcaagtggcatttcctgtagagacagacccagagggccaatttttgttttcaagaagtgttttttttaattatcagagattaaactattaaacagtccattaaataaattattcattttcttcccacttaatatttcagtgagccatgattagatgctatgatatatgatatgatatatacacacacacatatatataatatctctcatatatatatatatatatatatatatatatatatatatatatatatacatacacatacatacacacatgcatatatataattccagatgttaagctatcctgtaaattgtgatgagatttcatcaataaagtgtgaccctaattactcctcgtgaaagtttcaaaagtataaaacctttttcatcagatcatttgctattctagaaggtgactctatccttagtttcagaggacctgatttacagcacattgagatgttttatcccaacaactgcagtgccctaaacagaaaacatgccttcctagaattcactggtttgatagcaatctctgggtggcctgagcctcttaagacagttaattaagttatagttcatacacactgtgttttgctcatgataaacttacctaataagaaggaacattcaagacaagtattgtcttaattctacttcttcatggtagaaggggcaactagaaagacggctaagtcatgtgagcatgctttaaaaactgggatccagaacagatagcttgatacactgaagattacatttctcacccacttctgccttcattatgtttgtctctgttgaatttatagcctggtctgtacaggtgacagaatggatcagttgtagattgacagaagagaaaatgtggagggtaataaacctgtctgccttctcatgcatagagaagtggttacactgtacaatattgggctacaatacttacctttatgcaagagagaagatcgaactcagttgtttttcgtatttactctgttgttggtctctaatgtaacttgacttcctaaagacacctagcaatggacaccactaaaagaagtatttcttcatccccaatgcaaagttgagcactataagttttcagcattctgttcaagttgatggagcagacatcgagatagaactttttctgaaggcttgcattgggcttactgataatgtgtcctacttactgcttgcctgttaactttctaaaggttacctttctgctgatggactgaaaggtttctgagggatttctcagaagcctttcaggacgagggacattgaagcctaggtaactgctaaccacactctctctgttgtagtgtgcacaactaaaatcaaccccagagtggtaggaggagctgcgtctgttcacggtgagtggccatggcaggtgactctgcacatcagccagggacacctgtgtggaggctccatcattggaaaccaatggatactgacagcagctcattgtttctctgggtgagtattattgctattctcctgggattgccatcatgaaggtgaaatctgggactatcataagagtcaataaacactttgaaaatgtaaatgatcctgtttcctaaattaattctctctgtgtgggcagggacgatggtgtagaagagaccagtcctcatcatttggccacaatagaacaggggcaggagcagagcagattgcccacctctgccttttcattcaaacgcaaattatttccattgtcttcctgatggtgcctggtggcgaagcacagggccagagtgaagcttacaatcctcagctctctgaatcctggttaccctagtctctctttctctgcctccgaatgcttagagttcagcacacaccatcagaccagatccccgcacttagcattgcttttcgatattggccaaatgtgaacatcttagccggggaagtgtgtatctcgaggaaattcggttgagtgaaacctttctgtgctacttttagtgcctctgttgcttccagacacaggtttagaggctaatcgttttgttaatttttttccatgcatggatgcattatgcacataattcaatgctacacttgagatcaatagtcccctttgcaagcacatatgaaaaaacacagaaagtcccagtgacttttctttaaattctgcccaagacaaggttgagactaatacccaactctcctgagcttggagatgtgctggggagtagaaagaccatttatttaaagtgtccaatattagtgcaagaactaatccagtgatttcactgtagaggaaatatgtgactaaaagttttgagaataaaatcactttttttaccacctaaaggtagaaacagacacagagtataaataactgtgaaacacaaatatttggaaattgcctagtgatagatttttttttccattctgtttgttccttaggatagagacacctaaaaagctgcgtgtctacggtggcattgtaaatcaatcagaaataaatgaagggactgctttcttcagggttcaagaaatgataattcatgatcagtatacgacagcagaaagtgggtatgatattgccctgttaaaactggaatcagccatgaattacacaggtatatatatagagagagagagttttaggtgacctagataaaacattcacgttaggagactcacagtctcatctatggggtctaatcaacagacagacaaggaaggtctgaaaagatggcctcactctgttgagacagagagtttgccttagaatactagattagcgatccatacttatctccttgtactaaggtcaaatctaagtggatcaaggaacttcacataaaaccagagacactgaaacttatagaggagaaagtggggaaaagccttgaagatatgggcacaggggaaaaattcctgaacagaacagcaatggcttgtgctgtaagattgagaattgacaaatgggacctaatgaaactccaaagtttctgcaaggcaaaagataccgtcaataagacaaaaagaccaccaacagattgggaaaggatctttacctatcccaaatcagataggggactaatatccaacatatataaagaactcaagaaggtggacttcagaaaatcaaataaccccattaaaaaatggggctcagaactgaacaaagaattctcacctgaggaataccgaatggcagagaagcacctgaaaaaatgttcaacatccttaatcatcagggaaatgcaaatcaaaacaaccctgagattccacctcacaccagtcagaatggctaagatcaaaaattcaggtgacagcagatgctgtcgtggatgtggagaaagaggaacactcctccattgttggtgggattgcaggcttgtacaaccactctggaaatcagtctggcggttcctcagaaaattggacatagtactaccggaggatccagcaatacctctcctgggcatatatccagaagatgccccaactggtaagaaggacacgtgctccactatgttcatagcagccttatttataatagccagaagctggaaagaacccagatgcccctcaacagaggaatggatacagaaaatgtggtacatctacacaatggagtactactcagctattaaaaagaatgaatttatgaaattcctaggcaaatggatggacctggagggcatcatcctgagtgaggtaacacattcacaaaggaactcacacaatatgtactcactgataagtggatattagcccaaaacctaggatacccaagatataagatacaacttgctaaacacatgaaactcaagaaaaatgaagactgaagtgtggacactatgcccctccttagaagtgggaacaaaacacccatggaaggagctacagagacaaagtttggagctgagacgaaaggatagaccatgtagagactgccatatccagggatccaccccataatcagcatccaaacgctgacacctttgcatacactagcaagattttatcgaaaggacccagatgtagctgtctcttgtgagactatgtcggggcctagcaaacacagaagtggatgctcacagtcagctaatggatggatcacagggctctcaatggaggagctagagaaagtacccaaggagctaaagggatctgcaacccaataggtggaacaacattatgaactaaccagtaccctggagctcttgactctagctgcatatgtatcaaaagatagcctagtcggccatcactggaaagagaggcccattggacatgcaaactttatatgccccagttcaggggaacgccagggccaaaaaggaggagagggtgggtaggggagtgggggtgggtgggtatgggggacttttggtatagcattggaaatgtaaatgagctaaatacctaataaaaatggaaaaaaaattaaaaaaaaagaatactagattagactttgagagtgcagacggagagacttgccttcaggcttcttcaggtacatagaatgacatcgttttataaaataccgaagcaataagaataatgattgatattttgctttagtaacaaagggcttgacagcacagttgagcaaatgctacagaatattaaaccacattaaaaatgaaaggtggtgtaaagagggcctgtcttaccctttcccttgtcccttctgcttgggacatttcatatgtgccactgaacacatgacatgaaacacatgagaaaaagataacgaatatcataaagaaagagagcatgattaatgtcaggtgagataacacccctttctgggacaaatgattggctttctttctgcttcggtcagcttttcgctactgtgacaaaatgcctgatgtaaccaccctgtaagaaagaaaggtgatatgggctctcttgatgtcagaggcttcagcctgcagttgtgtgtgtgtgtgtggggggggggggtccactgctttgtagcccttggtaagaaatgtgcagaggagcaaattttcacctcctggatgctgggaagcattaatgaagggacggggctcctgatagccccttcaaaggcccaccctcaatgacgtcacttccttcctacaccttcccccccaacacacacacacacacacacacacacacacacacacacacacattctccattatctctaagtagtgtcacaggctgggatggagtctttgaacatgggcctttggggagacacctcagatccaaaccgtagtggcctctgatgactaaactgtgattttcaaaattagattttcagcggccaatatgcctgccttccaaaggagatagaaacgcagtgcacacagaatgctgggtgactggatgggggtacacagcactaagaggtaacaaaccatgccttctatctctgctttattctgaagtcaaagaacagagcttaaccattgcctctgttttctatctagtcatatggcccaaacgtgagtcaagtcacctactcaataacaggaagactgataacaaagatcaatacatctgatcagaaacgttaaatatgattaaacccctctaaagaccattttaactggagacttttagtttgggacctaacactctatgtaaaagttctagcctggtttctaattattttgtctgaaaagaaattctacttagtgtcagttaattttgaacttaataacattaatgaaattatgtacacaatagtagaaacaatgtcttctttatactccatacttacaaaaattacttatgaatcaagcttagtaataccacccccccccaggatttgtatgtacaattttggcttttaaatataattgtatataaacctatagtaattattcctctaaaacactaatatgacccttttcaggtgaagtacaaagtactcttcagaaagccaaggttccattggtgtcaaatgaagaatgtcagacaagatacagaagacacaaaataaccaataagatgatctgtgcaggctacaaagaaggagggaaggatacgtgcaaggtaaggcagtctcaagcaatcagtcatgccagattgaagtgagagcttaatgcatttgtacaaaccactgtaccattgagcagtgtccgagtgtgcttcctgttgctgtgataaaacactgaccaaacacaactcagggaaggaaagggtttatcaagcttacaggttacacagtccaccatagcggaaagtcaaggtaggcaggaactgcagtagagacggtggaggagtgctacttcctggcttctgtttagtcttgtgttccctacttttcttttgtgacaatgtggttaacaattagcagtggagaaagttccccacagtccaggctgatggcagaggtgcctcagctgtgttccctcttcccaggtgtgtgaggttgacaactcagattagccatcgcaagcagatcacttggtgggtttattttaggtaaactaaactctacaggaggaaggaaagctggataaaggagaacaattggatgtttggatgcttgtgagagggccagaatattatgtaaaattgctgtgagcaatacttacttaactcaggaaatgcctaccatgatcccggcatgtgtcttctttttctccccctttgacagggagattctggagggcccctgtcctgcaaatacaatggggtctggcacttggtgggcatcacaagctggggtgaaggctgtggtcagaaggagagaccgggggtctacacgaacgtggccaagtacgtggactggattctggagaaaactcaaacagtctgaaagagttcaactggtatcactttgtggccctggaagattattccatagaaatgagcttgacgtctctgatgaagacactgggatactgactcttccactgtaaccaattgaatggccttgatgtacgtaagaacacccagaaagaaaactattattttcagaattcctgatctgggagaaccactggttgttttctgcatccagctactactcaaggaaacaaatacagcaaggagattttaaaaataaaaacacatcagatatataaggaaaatatcaagtaagggtgctgtctgcctttttagtctctgtgacaaatacctaaagtagttcacaaaaggaaaaatttcttttgcacacctttcctcaggtttcagcctacgatctggttggctggccccattgctttagcctgaggtgaggcagaaccatatatccataggaggctgtggagaaggagtctgctcagttcatggtaggcaggaagcaaatggaaacaggaatgtattggggacacgaatggtccttcaagaatatactgtcaatcatttacttcttccagagacatcctgctccctaacctccctttccttcccagataacacctctgtcctagctggccccaagagatcaggtagaaaggcagaggaaaccatataaagagttgttaagtgcaaaatcaaaaccagaaggaatgcagacaggagctcaaaatgtccatttataagaatctttttttttctctgcctatatgaatccccctcctgtataaaggactgactcaattcagtgatgggttttgagaagtctgtttgtgtgtgtgtgtgtgtgtgtgcgcgcgcgcgtgtgtgtgtgtctgtctgtctgtgcacattcatgtttaggtatgtgcaggtacctggtgggggcgatcaaccttgcattatttctcatgtgccatctaccctagcttttcaaagacagagtgtcttactgggattggagacttgggctaagctagctatctagcaagtccagggaccattctgtctctacctccccaaactgaaattaagaagacatgccatggtgcttaatttaaacctctctctgtctttgtctctgtctctgtctctgtctctgtctctgtctctgtctcgctctctctccttcctccttctctccctcccactctctctttgggtgtgcacgtgcgcatgtgcaagtcatagtgtgtgtgcagggcagtggaccatcttcaggagtcaatgctctccttccatcatgtaaggtccagggatggacattaggtgttcagatttggtgacaaatgtctgtaccttcttagccacgtcacaagccagctgttccgatttcctacagatgctgggaatcaaatctgagtcctcggggttgcctggcaagcatcattactgactgagctctccagtggccttgtcagtcttctctctgcattttcccaaactggcttggacaagcaccattgcaggtgttaagtgcacacttcctaatttccacatgggccgagtataggaggagcaattttccaggaagtggtcccttgaagacacaccgtactgatttgcttgcctcggaaagtatctcagcgtagcctgcactctttttgcagtgttaggggaaagtacaggtggatggagataaggaagacaagccaaaacctaccaagatctgccagtgagtgggagtttacaaagctgagtaatgaatgtgctggacggaaatgtgtgttgaaatcgtacatactacgggggggggggggggggtggataatttgggagcaaatgtggtttcaatagaggctgcagcctcctcaaacagttctctgtattctgagtacctgacttttgtcctcacatggggcaataatgtagtatttggactttgtccccgtacttttcagtcagcgttgataactatacaagttgtccaaatgaaaagtatttattgtgcccaattatgtcagagtgtcttgttgagcttggggaactgaagcgccagccaataaattatgaaggtttcataaggttttctgttgatttagtacgaaccgaagagaggagctgcacaaaatctatactttcaaacaaagatgaccatgacacaaagggttctaagaaatgacaacgaagaagagttagcagaagctaagagagtggcatggaaaggaagtggccccaagcaagacaaagcaaagacagcaaacaagcaaaagccagagatcgatgtcactgaaatggcacgagcaggctggattcaaaatgcttctagagtaagacagaattgacatcaaatggggtcacaacttcacaacccatgaacaagcagcgccttttataacctatttattacatttcacataggaaatcttttataacctatttattacatttcacataggaaattgaggaggcattgctgtcttctctgagaagtatttaaggaatgttttcgtcttaattttttttcagaacaagtgcaacatcttaattctgaatatctagtacctagaaaatgctatgagctataaggaataagaaattacgctgagcagattcacatctccacaccaacaagctgcgaatctgtatactttctggcacttttctcacttaatcttctctctcctggagctagctc

SEQ ID NO: 3 - DNA, 74000 bp, Mus musculus
  source 1..74000: mol_type = genomic DNA
tgtaaactagatctccatcttagattaggctgtgtgaaccgaatttatttacatcgttagaaaccaaatagaggcctctcggctattgttctcagattgctctcattggtcatccctgccctccctctactgaccagaaccttgccccaaaacagcctgtaataaacatcaacgctggcttagcttgggctgctatctctggcggagagatctttaaaggatgtatctaaatgcaatgtttgagtagcttcagagagctctaatagaactgtaaatatccccggtttaattagcagtcctgcagttcggtaatggcccatagctctctgagccgagcctcttgaggtttctagacttcagaggctgcctgcaactatgctgtgtggacctatgaaattttccttctcctgtactctaaacccccagctagcctttcctagacacctactcgcaattattgcaaatccataactgactactatcctccggatttctaaaatgatccagtgtttcagcttaggtctcaactcagagatactttagggctcagattggcatcctgagaattaagtcccctgggaaaagaacaataaggaagaaaactctacctacattggagttgatgtcattttttttttccctccaagctcaaggtgatcgcttgctttgtggctggttggtgggggaggaggggctgtacgctagttatcagcatttctgaaccagctctctcaaccgcgacaggtcagccaatcccggcagtaagcttttacttgacaggtttgttctgggctgacagccattgactaggtgctcagataagtcacttggctgagtctacggtaggtggggcgcgctcaccagttcaggggcagtgactggaagtttgttgcaacatcggtaagcctaaccagccagcagcaacaggagatacccttttgccccgcgagtacagatctagaaagggttcacctcattaagcgaaggagatgcgtcaatcccccccacccccgccccgcgcctccccctagggcccggcctcttctcccacggttgggaacgcgcggtgtgggcagatccagaacaggagtctcgtgtcccggccttctggctagctctatgggttacaagcgaaagggaggaacagcttggggactctccgcgtcagcgtgcacaaaccggcggcggccagcagagaggggtggcgggggcacgtgcttggatgtggctgcttgtgtaaccagctccccaggcgctcggccccgacagcgctcctgcggacggctcgtggatgctattctctgctccgatccggcaagagaggggtccagcagaccacacgggagaaggaggcgggggcgatcacctaatagagcagaggggaccaagctcctgccccaggagcacacagataggggaatgggaatttggaaagttccccaactaggaccacacgtgacctcctcctgaaagtagttccgaccgcggctcatgtatccttccacctcgcctttgagccctcccaggcctgctcgccccgcccactcgctggctgcagcttccgaacgtcccatactccacacccgggctcagtaaccgggtcctcgaacatgcaaggtccgacagggtcagaacctggccatcgcgatccaattctgccgggttttcatagcggccacgaagtggggattgggggtgggggcttagctctttgaagactgagcttggctgtgatccggtagacccaccgctgcggggagctgcgggtctcatcaccgggcggtggaggggtgtgtgtgaggtgcactctattcacggagacccactttgtccaaccaggggtgtcctttgggccctggaaactcaggggagatgtgaatgtacacgccccgtatgcacaatcatcatgcttggctgggagcgttcatctttcgggcaaatgaacccagctgcctgggaagcaagaggcggggcagggaaccggagcccgatgaggtgacccacgcgggagacacaataggggttgttctttgtgcaaagactgacaccttgaggacaccgtgagggggagaggtgtgttatctaggtaaagactgtcgccgacaaatcctagcgaagcactgcaatctgaccacagcgcagggcagggaatgaaagccgttccgaagaaacgcagggacagacgcaggaaggataatcctgcccctgaggctcccggagcaccgaccaaggcggtcagctagtgcgatccacctgtgagcggtcagcgattgtgctcagcgcaccctcactcggccccagcctgttgtacctttgccgggtctctctgcgctgaggccaaagccggcgtagctccgggagcgagccgcggacacactgggcatgctccgcggcgttccccgcccctgtcccttccgacgccccgccccgccccgccccgtccccggctcagcgcccgcctcccgcccgcctcccgcctcccctccggctttccgaggcgccctgctctcccggcggggcggcggagggggcgggctggccggcgcacggtgatgtggcgggactctttgtgcactgcggcaggatacgcgcttgggcgtcgggacgcggctgcgctcagctctctcctctcggaagctgcagccatgatggaagtttgagagttgagccgctgtgaggccaggcccggcgcaggcgagggagatgagagacggcggcggccacggcccagagcccctctcagcgcctgtgagcagccgcgggggcagcgccctcggggagccggccgggcggcggcggcggcagcggcggcgggcctcgcctcctcgtcgtctgttctaaccgggcagcttctgagcagcttcggagagagacggtggaagaagccgtgggctcgagcgggagccggcgcaggctcggcggctgcacctcccgctcctggagcgggggggagaagcggcggcggcggccgcggctccggggagggggtcggagtcgcctgtcaccattgccagggctgggaacgccggagagttgctctctccccttctcctgcctccaacacggcggcggcggcggcggcacgtccagggacccgggccggtgttaagcctcccgtccgccgccgccgcaccccccctggcccgggctccggaggccgccggaggaggcagccgctgcgaggattatccgtcttctccccattccgctgcctcggctgccaggcctctggctgctgaggagaagcaggcccagtctctgcaaccatccagcagccgccgcagcagccattacccggctgcggtccagggccaagcggcagcagagcgaggggcatcagcgaccgccaagtccagagccatttccatcctgcagaagaagcctcgccaccagcagcttctgccatctctctcctcctttttcttcagccacaggctcccagacatgacagccatcatcaaagagatcgttagcagaaacaaaaggagatatcaagaggatggattcgacttagacttgacctgtatccatttctgcggctgttcctctttgcttttctgtcactctgataacgtgggagtagacggatgcgaaaaatttctgtagttggggtgactataacgtttaattctgggcgcatttctagatcgtgcatattgtgtctcttccagtgtattcaacctagggagtgttcggctagacggaactcatgcctccttgcaagtgtcaaggcacggattgttttcttgtcgagctctgtggtctcttcttaaaatctattgtccggtaatacagagtactgtacactggattagcgagctcgtcaatccagccttctaaatgaactaaaaaaaaaaaaaaaaatagacgctttgggttgtgcatatttcgattagatcgtgacttgggccctagatctagggtgtagatgagattaaaatgaaagtccgtgctgcacgtaggcatgttgcctcagaatcttgcagtgattgtttttagtttcctgggttgcattggaagatttttctgaaatgtgacttgtggactgatttgcataactttataagcatagtcatcgtcagcatggacggtgcacatttgagggtagaagtagttttgtgtgtttaaaccgacgtaattacaaatttggagcctgcatttggaagttttgtagtttaaaatcaatagtgccagaatatatatcattggagcttttaatagaactttgttaaattagactttctttcctgatgagatgtgggataggatatgaaatttgcatttcagtggtatgttccattcatttgacctcgccacacttgatagaatggagatcgaatagtttttgatcccagatgatgatgtctgtgagtccagggatgcttgctgtgtgtggcaaatgactgcagaggttaaactcagttctcacatgaaatttaagtagtagcgtgaattcgctgctcatattttttgtttcaggtgctctgactcgttggcttatcagtgggttttagcctgatttgtgcagaacgtcaagtataatttagcccgctaatatccagacaaactggcaggaatggatcactggctccagattgtaggtatgctagctctctgcatgcagtaatttactgagtcagcttgttgaataagacagaaacccaagaattttaacgttgtatagttctgattgtctaaaagttaggcctttgtacttcaaaagttcgtggtttaaagcatttatctttgcagtttcaggtatggtgctggttatgatcaggggtaggaaagagagccctcctcttttgcagagtgggaagtgttgacagaaattgagagctttggggaggccagtgtgcaaggtgtgcgaaaacacaagttgcaggtctgaagtgactgagcctttaaatgattgggccttttggtacaacccaaaattttgttatgtgacttgtagcattgaacagtatttagttaaaattatgtaattttataattggtggtaaggtattctttgtaaaaattattttttagttaatattagtagacattttcttaaggtggaagggcagcagcaacttgtctatacatgtcctgctaatagatcactatggagaactttgtttattaattaatttattattttttttgaagaaatattttcatttggaagaggtttgatggattacattagtggcatataaaatgcatagctaattttgattgagttttttgttctgacatgaagatgaaactctcactgaagagctggcttcacacaggctaattttctttcgagctaaaaatacagtttgaaaggaatctttaatttccgagtttgcgttaattgagaagctttactcaggatgaacttgaccaaaagataattggtgagcattctgagttgtcacttgcttcaaggcagtttactgttggtgtgaattaatgttaataatctcgatctagaaggaattagtcaggtatctatttaattaaaaaaaaaaaaacattcaaagaaaagtctagaatgcctggtaggcagacttcttgtagagtccaaagtacacttctgctttgccttcctttccccaaaggacttcaagattattttgtaaattttttttttttgtctgagcagtgacgtgattgcagttccttatttgatttcatactgcatgggcttctgcacgtgcagtttatgtggctgaagaaatccaaagctctccctttagttatggtatgcgtagaaagcagcatcaaatactgcatcaaatgactaataacaaactcttaatttctagagctgtggtttgatggagccaaatgttgatgtgagaacgagtcaggaggggaaaaagtcagcgagccttgctcttctgagaggtgtgcgccccttagttctgagaagctagggagagggcttgcttggttcctcatcagggttttctgtgtttggggcagggattaaaatggtcattgaagatttgtgctggtcagtgttggcaatgaggttggccaagttaaatttcattatgaaagtagtaagttgaatattttcatttgctgaaggcacaattgaattactgtaggaatgtgttggttggaaattattattttttttttaatatataaaactctgaagtctttttttttttaaagatttatttatttttatttatacaagcacgctgtagctctcttcagatacaaaagaagagggcatcagaccccattacagatggttgtgagccaccatgtggttgctgggaattgaactcaggacctctggaagagcagtcggtgtgctcttacccgctgagccatctctccagcccaggaaatttttcttaccgtctggaaagtaggctgaatgtgagacagcaagagttaatgatgttaaagcacagagaaggttcaggtttctggtacagcttttttgtttggtatatttacttttcagatactgaccacaggaatattagtaccttgcagagagaatagtgattattgatattagctcttttgttaaaggcaagtgattgttgcaggtaattatggtttttctttataagtgactctacaaacttcatcttgcctttttcatagtcatttgtgtctttcctgaaaaatacaactagtatagtaagttgcaaatttcatttttcctaaaacttaagaggagtaaagtgcatcaaaaggagtaattttcagttatgtgtaattcaatataaggaaaagaaattttattactgtcaattttagaagtttacacagcaatagagatgtagactgagattccttataacgtattaaataaagattaaatgtttcaagttaaatatgatgaatataatctggttatttctagacatagatttagaaagtggagattcatggcctttaaaaatctttattttagtaacaggagaaaggcagatagtgtattttcatttttttgtgtggaacattatgtctaatttatcataattatataaaatatatacctaagtattatgaaatagcttacctcctggaaatgaattcagttcttatttttaaatgttacaacatgagttgttggtttttttttttttttttttttttttttttctgttcatttgtttatagagtgacagtattttttttttttttttcccgagacagggtttctctgtgtagccctggctgtcctggtactcactctgtagaccaggctggccttaactcagaaatccacctgcctctgcctcccgagtgctgggattaaaagcttgcgtcaccaccgcccggcaagtgacagcattttaagccacttctaaaatagtcactttaatgacttgattagaaaattgttcatgtaggaaaatagaaagtttgaacattcactttagtaaatattcagttttcaatactttatgtctcagtaagctccaaatgtaggcagtgtttttctattgataacttatgtaaattcagagttgacagggataagtttagagtcctgagtatgtaggtcaggttttcatttctgtaggcctattccatttagaagtattctgttctatgaggcagtgttagttattagtacagcttaaaaagtttacttgctcgcattttttgtttttccatgataaactactggctcataaaacagaagtttggctgcacagagctggtggctcgcgttttgttcccctttcttctccctgtagataaaaaagttgattttcagaaccagcgttggagagttggttttgtgatccactcttttgaaaagttacaggacttgttccaaagatcatgatttaggtatgacactttgtgtggttttatcagaagtgatttctgtaaatattcagggtttttttgtagttgatttgtatgtggtagttttgaagttcatgcagttgctgagctattagtgatgttgaatacagattaaatagctttatgttagtatttgctgatttttttttttataggtcttgctgctgttgcttaaaagtgtctaattgtgggatgatggttctcaggtcctgaagctattggcagagatgaaattaggatcctggctttgatgtcttaccttcccattctgtattctctgctatggtttcagaagggacgatatagcagtttcagcttctaaaataatttatactggaacaaaaagcctgtttcttggaggaaggttactatcagagtaaacaactaattttgcctgggaagttttaattacatttagaatagtcaggtatactcttctattgcaacggacatttgtggacatctttggagtactaagacttaacacttgtttgtggtgttaactttggagagatgcaaactgcagttgcacccactaatgacctgtatgtgcgtggtgtggaaaagcttgcaaatctgctgttttgtgtaattagtcaaacagttataatcagaaagccaacgcttaatacgaacagtgactttaacgtaaatatttgaatttttctatgtgtaggcatgcatgtgagtatcaagttaacaaacttactagtctggaaaatggattttctccttaaagaacagactactttggaagccattttaaaccataattgagtcttttaaattacattgaaatacggctggtcttatctatactctgaagttataattatttatcttagttttaggtagacagggtccctgtcttggctctgtcacagagctgggtaactatgctatttaatttctcttagtcttactttacctgaaataaaggaaggataatgatacccaccacaaaagtttatctaataccagcaaggcagaggaaggaggctattgctaattcctaggcagtagccttcagtactacatagtgaatttgaggtccactatagttctatactgtgagttccaagccggctaaggctccatagcgagaccatgtctgaacaagcaacaatcaaatcacatagttaatccccctacagggtttctctgtgtagctctggctgtcctgggactcactctgtagaccaggctgacctcgaactcagaaatccgcctgcctctgcctcccgagtgctgggattaaaggcgtgtatcaccactgtctggatgttttcaacttttaagggaacatgaaaacttccttagttttcacataataactttaagaactatctgttgtaagattaaattttcttagtattcaatatagatgaatataaatagcatgacatttataatatgtgtgcttatatgatacgatacatagcatatatgatctatgatacagttatggtatgcttgtccttaggaggcagagcctcaccatgtagccctggcttgtctggaacttgtggccatgcttggtcagtatcacagttctttttcacaaagagtattcctcaccactaagaagtaggacttcgaatatgtaactcagttatataaactctcaggaaaggtacattagcatttcctttctgctcttccagctcacatacatacagcagacaatacagtctgcagtgagctactataggaatttaaagttactgttaaaccttgccctcagtttcacagtgagccaggattttcagtataactacaactttgaaatgggaagaaagagctagagagatttttttccttcccatactgcatttttaaaaattactcatttatttggggttggggatgggaggaatggaacagggcatacgtttggaggttagagaacagctttcgggagttgattctctcattcctctctttgacactgggaatggcagtcaggttgttagggtagcaggaagtgccttttagccagctgagccatcctagaagctcacatgcttttttcttatctaggggagggaagaacggagtaagcttaaggctcacctacctgcacgtgacagggactagtttttccttcctgtgcttgtggtttcctgggaatggagcgccttaatttccagggctgtttcttatttattttgaaatgcagagtggatgctgctgtaattattcatgactgcgctcttgtaatttttatttattttttgctgagctttccctgagtattttagctgttttttttttttgtagcagattataaaattgagttgcatatctttatatctgaacctatctcactgtctgaacgagtttctatctttctcttctgtctgtgtacctgtactataagcacataggtgttatatcactagaggaccagtgactttcattgcatggtaacactcatgtaggttttcaggctgaagtagaataatttcctccctccctccctccctccctccctttttcttccttcctagtgatgcagacctcatttgtatgtttgttcttgaccctaggagtaggatgcagttactcaaggtgtatggaagggtatgaattctgctttgaaacagcttatcttttaaggagatatatttctaaaagtgaattgtaaaaatcaatatttgtttaacaacgctagaagcaaatttttgctttgaaacaaaatgcagattttttccaataaattttttctctttaatgagactttaaaaaaaaaaaaggttggtttattagtgtatatgagtacattgtagctgtctacagacacaccagaagggggcatccaatcccattacagatggttgtgagccaccgtatggtttctgggaactgaactcaggacctctggaagagcagtcagtgctcttaaccactgagccatctctctagcctgagacttgttttttgaatgttttatgtttctctcaactaaatagaattaaaatttttcttttatgttgagaatgaagtttggggagatattattaaagttgtccttaggactctgtattacattatctcctatcaaacaaaccttaatgtactatattctctgtgtatgtgtgtgtttatagtttataggctagcaatcaacattggtgtctcctctgttgctctgtgccttgtttattgagacagggtctctcattgaacctggagcttgccaatttggcaagactgtcttctgtctgtgccctccagtgctgggatcacaggtgtacacaccatacatggttgtacacaccacacatagctgtacatactatacatgggtgcgtaccaccatacatggttttgacattggtgctagggctctgacctcagatctttgtgcgtgcatagtaaagtactttatccactgagccattccctcagctcccatgctatatactcattgagttaatagtatttacaggtgcattatatcaatagacataagtcctaaataccctaatgtaagaaatatgttgatttgttttgtagacaaggcccagtgaattagaaaggcagcctgctagaatgcaaagaacatagcctggcattagccaggccctggaattagattcctgccctagttcccatctttctaatgaggggtaaagctgtgacttgtaggtgtttctttttgttgatctgaacttttggtaatgtctagaaacattttggtgaacatggtcatacctgaggtgacagaaggcctacttgcacttagtgggttgaggctactgggctcttagacatttgcagaacctgggatctgtttaatgttaggggaagtgtttgatatgtagatatagtacctttgccaaggtcaactgttgctgggtggtaaaaactgggcccacataggttttctgactattgtggggcctttctctctctctccccccccccctctcttttctttcttcctttcttccttccttccttccttcctttcttccttccttccttccttccttccttcctttctttctttctttctttctttctttctttctttctttctttctttctttctttctttctttctttctttctttctttctttctttctagacatggtttctctgtaaagacctggctgtcctggacctcactttgtagaccaggctggcctcgaactcagaaatccgcctgcctctgcctcctgagtgctgggattaaaggcgtgtgccaccaccgcccagcggggcttatgtttcttctaacaactttgtccactaaagatatctctaataagaaaaagatttatttgacaaggtctcatgtcttttagcattttgtgggagtttgaacctctgctgaaatgctgtctttgtcgctgttctgaagagtatgacagggaatgcaaggtggatgtgcagtcaggtgttttgctccttgaagctgaagcaggaggattatttttatgtttgaggacagcttgggcaacctcatactacaccttgagttctagggccaggtcagactgtctcaaaggccaggttcgcgctcacccccactcagtgtttgaacagttttctttgtaattgtttagggagttactttgctgctagtgtctttaatgttactgttgtttaactgcttctttagtcttagatttttttttttttttttactctaaacctttgtttcttcagatggggtgaagttaaatgtgctcatgtgaagaaggggcaggtttctgacaaagaataagaaaaatcatgaaatttttccagatgaagagaatgggttggcctgactgaagttcccgggtgctggtcacctgcttttctgagccttacattggttgttagcctggttactggaaattaccgtgatgctcctgaattgggcatgcgagtttgcattagtccaaagatgcacagaaactaaagcagtaaaaaggacagagaatttcttggtttatttttagggatcacaagcataattataagtgttctaggaatagattagttgctatgttggtttctttttcctgtttaattttacaatttcatttgctgctcactttacaacagaataaaatgtccaaggccttaatttttaatttcttaggaaacttaagtactgatttagagttttggttcttgaagttgagggcagtgtttaggtatgtatactgataaatgttttgaaaccgacatgacttgtcatgatagcttcatattcaccaagaaaagcaaatagtatccattgctactaaatactatccattgctaaattgaatagattaacagtgattaagtctgaattaagagctcatttgcttactaaagaaaattccttttataaagagattactaagagaaaaattttgttgtttgctcatttgttttttcatttcttcaaaacttcaagttgttttctaattcttggtgaactcaaagaggtatcttttttgaataatttatatagtgcttcataattgaaattatcaaaaatacatatttacctataaattaagatttctccatttttttctcttactatttttttatatagtggacttaaagtttcaaatagggacaatttttatgattgagattttacacaaatcacaatcaacaagaacaaaaaaaacaattgaaatattagagctttgaaatgccttcatctgaaattacttctaggccatctattgcacatttaatacttaatgtttatattacttatgtgagatagcacacacacatataatgttatatgttacatataatatatattaaatatttgggacaactttccagtttcctagaccatgctgccctcgaactcattataaacatcccatttttcctgcttctcaagggctatggttataggtttatattagaattccaggctccaaatacttttgatatattaatttcatttattttggagtattttgctgatttgaaattgaaatggaatagaaataatttgaacaattttcatactaaagtgggcatagctttgttgctgtacctttgaatccataattattggcttgccatgatgttacagttgcagtttgaataatgtggtttatatttgtatagttggcttgtatttgttgtactatcaattaatcaatgtgcactgataactagtactagcttttggtgaaaaatctgcatttaacagtttgtcagattttatttttaagtcagatactaaaagagaatctttttttcatagatttttattatttaaatgcattttatatatatcaaacataataatactgagtattttaggaatcaaataatacataaaaatttgttcaatttttatattcatatctaaaagagaatctttaaccatatcagctaaccattaattctttatcaataaattatattaatattttttgtataaacaatttaaaacaaagtgatagaaacatcgttttttattttataaacaaagttactactaggtgtgctctgtggcctgtgaccttagcaccctggagctgtccccaaggaaaccagaagtgcagttactcttggccacattgtaaggtgaagcttagtctagaccaaaagagacagaaccaagacagaaaaacaaattacctgcttattattaagtacgattctttcccaaataaagtgaaacttaagcttgaaattttcatctaggcataaggttgctatcttgggttctaataaaaattagtggtggcaaaaatcttttgttactgttttcttgttgctgcctggggactgaagacccagtcttcagtagctttggtggtctggtttgtagggcatttgatgaaagaacagtattggaaaactgtgaagcactttctcctgcattgtgtccggattgtggtcatgattgcgtgagtttgaaaatatgagaaggtccttagaaagatgtttgtttgttattactgcaaaggtcataggttgtggttgtgccgtgtccagatacccaggcctgccatctgttcctcagatgtaggaaaaagttaaggtaatttagtggtagtaagtggttttgacacctttgtttttggaatcatctaattataagttttaagacctaagattaagttttaggcctttgtaaaaatgaaacttacacagtatgctatcatttgtgcaaaaccactaagggcctgcttcagagctgaagatggctgctccgagcccaactctttactgaagtgtctgcatgcttcagcacaggagtgtcacgtgtcacgtgtcatgctttcacctgacgtactaaattacacaaaatgttggggttttttaaactaggttttctaacacgttatgtaagaattgataatacagaactcagtgagggcactgacttagtaagcagttttaaacatttcttttaagtatttttttcaagccctgtgctttttttctctcctacaacttaattcttaattttcatctgcgtttgaaggagcttcctcaggtaaaccttttctcttgcattatacttgcatgaaaatgatgttagctatgctgtatttaaaatctttaaaatgcctctatatggcttaatataatctgatgagttttgtattatagttggagtttttgatgtgccaagtggttattgtaaaaataatgtctgaagtttaaatatttgaatttgtatttttttttaaagatttatttattgtatttatatgagtacactgtcactgtcttcagacacaccagaagaggacattgaatcccattagagatggttgtgagccaccatgtggttgctgggatttgaactcaggacctctggaacaacagtcagtgctcttaaccactgagccatgtctccagcccgaatttgtattttcaaacccaacttcgggcctggggtctatagtttttgagtgcaagcttcttgtcaggttaatgtctgggaggaagaaggttttgtcagtaaagtttctgaacttttgactagaataactgactttctgtgatgaagactaaactacaggccatgactgcacaggagtcagggcgtacatagatgatcctcagattttgggcagcttttacccaaacattgagtttagaaactgttctccattcggctcacttcgtatttggatagcatggagtattaaaggagttctcattattgtcatttttattgcttccatttaactggtatttttgtcttttgtgtattgagctctctttctgtctcccccctctctgttagggcctcactgtgtaggtctagttggcctgaacctcacaagactcccactgcctttgcctccttgggttacaggagggtgccacctcacttcagccaaacaaaccttgtatgtttcagcactttatttatcagttgaaaatactgactttcttctttttttaaattttagcctttttttctatggctttaaaatatattctttgaacataaggatgataaattacaaaggtgctttctcccacagagccagtaattattttcttataaaaattgtacccctccaatcaatttctttaaaaagtagtttatttaaatgtgcaccaaagactcatttcttttttttccaaatatttttgtattttatatgtaagattgttctgtctgcatgtatgcctgcatcaagtccagtggttgtgagccaccatctgggtgttgggaattgaactcaggatctctgggagagcagccagtgttcttaactgctgagctatgttcccagcccctaaagacttgttcttactttccatctatcaatacagttaaaatgttaatagaaaagtttcatattctaaagtgaattttaactagcatattttaaaatacaatttcacagttcagggaataattttataattcaaagttgattataatgatttaaatgttttcagtgggaataggaatccttttacaatattaaaacaaggcaattataaaatcatcaatatgactctactctagtaatgagtgacagtatatatgtcttcatgatttaaaatctacataattacttccggttcattttatttttcttcagtccaaaacagtccataggggcaaatttatttataccacttgcaatagaaacgaagccatttcatttgatttctatgttagtaatacagggtaaagaaacattttatttcatgtattacatcaaagtcatatgtaaagtgaactaattcagcccacagatttggagtttttctgttttaaatagtctcaaagtctgctttgaagggtaaaattttgactgttaaaagcaactaaaaattttgcaattttggctaaattgaaggagactggtccatcggcaggcaggacatactgcgtgaggaccctgagtctcttcattctcagtgctgaacttgctaaggcattgcaggtgaatgttttgggtttttttttatttttgcaagatgcagctactactaagttaattggcactaaatatgacaggggctttgttctcaagttactggtccatccttcaggcacataggaatatctctcctgtcgtaagttagagactttaatatcttttgaatgattataaagtagctaaaacttagtgttttgtttctaagcagcctgttgcaggagtaacttagaaatctaatatcaaatatgaagattagagcattagagagatatctcttatagctttatgttagtgttgaatcaattacagctttagtttttatagtcttgtgtgtaccattatgatttttctgtagacagtttggagtttgtgtttgccttagttctcataaagttgttaatatgcctgatacatgttgggcttctgcacttttgtgcatattttgtcattagtggtcaatcttagatggcttttttagtggcaagaaaaccaaagagatgccgggctggtggcgcacgcctttgatcccagcatttgggagctagaggcaggcgaatttctgaggccagcctggtctacaaagtgagttctaggacagccagggctacacagagaaaccctgtctcgaaaaaccaaaaaaaaaaaaaagaaaaccaaagagtgttatgcataaaagtgtgtatgtgtgtgtgaaccaaagagacagcgttgtgtgtggatgtgtgcgcacacttgaatgtagtgtaaatgcagtagagctttgaaacaggaaaccagtcttttcatattttccagagacctaggaagaagctaattgttctatataaactcattagaaattaaaagttttagaggctcacatatgtaggcttttgagcagttgcaggtacattatttcataattaacttctaggaagaaaaactgggtgggaaactagtagggatgactagtaagcaagccatcggtaaatatgcccaataaaccttactttggatttcggagttgctgactttaaaaggaacttaataaactaagtaaaccttatttgaaattcagaatacttttttcattgaaaaattgaaagttttattgaaagtttttcattgaatttattcatatccagatataaataagattggaatcacttttaaagagattctaaaacttaaggattcagtggaaaaaaatgtgctggggatacccagggacttccagatgtaaggcagagtgctctaccattgagctatgaaaccattcctttctttctttacttttttttttaaagagatttatttattttatgtatatcagtataccattgctctcttcagacacaccagaagaggtcatcagatcacattacagatggttgtgagccaccatgtggttgctgggaattgaactcaggacctctggaagaacaagcagccagcgctcttaaccgctgagccatctctccagccctctttctttacttttgagtcaagttttccttcactgacccaggctagtcttaaaccctggaggcccagaacttgtgatcctccagtctcaccctaccaaatagctaagcattatatagccctgcaccaccatgccaggttgattctgtttcaaagggtgttactggcacttgggtgtggtgcctgtaatcccagtatttagggaagacaggaggaacaagaggagttaaactttcctgctggcaagttgcagaccagttcaggctaagacacccctcttcccacaaaaagaaagtttgtcactggaaattaagttagttaatgtatatgcttacattctcatgtatgttgttattgcatagccattgtcagtgtttgatacggttttcttttcacaaagagttttttttttttttttggtttttcgagacagggtttctctgtgtctggcctagtatttgtttttgtttgtttgtttttttttttttttactttatttattatatgtaactacactgtagctgtcttcagacactccagaagagggagtcggatctctttacggatggttgtgagccaccatgtagttgctgggatttgaactcggaactttgaacctttggaagagcagttgggtgctcttacccactgagccatttcaccagcccttatttatttatttatttatttatttatttatttattttttgagacagggtttctctgtgtagccctggctgtcctggaactcactcggtagaccaggctggcctcgaactcagaaatccgcctgcctctgcctcccaagtgctgggattaaaggcgtgcgccaacacacccagcttgccctttctttcttaagcattttctttgtaatatgttacatgcatgttagggctttcagtgtcccttgttgaaagcactccagtaatggtaaatgtaggttgttcttgatgtctgctgacttgacaggccatgacgaggcttttccccttcaggctttcccttgttcttgactatgaccccatttatgcatatatgcctgagtaaattgaactacttgacaggcatccctaaacctgtgtctgttttatgtaaatcctgtcctttctgtgtgtctttatgagttgcattgggctcttgttcctggatagatttctgtctctttcctgcagttctctgcttggactgttctagccacttaagtatatcttttctaatataaatcttattttttatgtgtatgagtgttatgcctgcaaacatgtctctttcccgtatgcgtgtctggtcttcactttgatatgggtacagggaaccaaaccggatgctctttctgcaagagcagcaagtatgtttaactgctgggtcatctctccaaacactcctgtgttttcttctgtcaccagaaggcgtgtgtgagtgctacccaacataatactcacttggtgatgcttatacatacttccacggatccctctgaaaacatcttcatttaaaaaatacagtagtacttttagtgccatggtaggtctgtgtgcctgtctttcttgaggacggtaaccactgcccggccctacagactttttaatttgtctcatttattcttgcataatattatttagcctgtccctctatattattcctataagttaacattttttttctcaaaggctttgagagttggtgttaaagattcttggccattacagatgatgctctgcctttgtagtacctatggccaaagccttctcatgacttggagatcaattactgagttatatgtagaaggcaaatgtatccagaatatgtaggcggaggtcttaagtggttgttttaaaggaggtaacttggtatagttgatgtgaaaatcttgtaggtagttatgagatggaaccccagaacaaatgagagctagaaagatggataaaattcatggaagtgtagatttttagttaatcggaaataaattctcccagaatatagagatgggtttttatgttaactggttttgaattgaaactaaggacatgctaaggactaattacactgatgagaagaaagcatgtaggcttgagcctcagtcgcgtattctgacatcacagctgtcagggatgaggttatcactgcccgccgagtcactgtgggcagtaggaacttatagaagtctaaggatagtgagtggctgactgtccaggctatagctcaaggagcagacaagtacatttgacgaccttttataatcacagctagcgtgggaaaagctaatgttttcaaatgcatgcatatttgtgtcattgtatattctaggtatttccttaacttaataatttagatatttatccaaatattattgctatgggatttcctgcagaaagacttgaaggtgtatacaggaacaatattgatgatgtagtaaggtaagcattcttgattttctatttcttatattaataaattattttgatgtgttttatttagaaaagatcccgaaaacacagaccagtatttgcattttgatgtgttttggtaaaactctgaaagttttaacctaaagcacctgacagctctcactcggctggatgcgtcactggatgagaactggctagttatatagtcgtgtttgtttatgtcatgaagatttttttttttgtattccataatatgtctcttaccaggttattctctggcttgtattacagtacaaggttttgactttgtatttgggttaggccttgcttaagtaggtttgtttagttattcaccctgcggtatgagtgaccgatgtgttttatgtagcacttatacctgtagcagtgtttgatacaatgattttggagagacttgctggacattcattcaaaagagtaaatgaagagtatcataattttacaaaatttccaagtgtgattgttgcttagttcagaaaagtgtttctcaaggcccacttaaaaaatttagtttcagaataaaaatgcaattgtatgagtaaatgaacattaaatttttgttgcaaactatcatagtttttaacaattcattaatactgagtcttgctgtatttgctatgctggttctgaagtactttaatgaatttaaaagttaaaattctggattatttcctttttactcataagtgagcatctttcctggtgcttttaactatgtgtgattatgcagatttaaataggtgggagagtgctttgtaacttagagtgtcatgcagatgtaggatgcttttgtggtactgcatgactttatgtctttagatgagctagtagtcagtttcctaagtggctaatttttactgttttaaattgtgcttgtttggataatttattataatattgtttatgttttgttcaggtttaaatttcaccatctataaaagtctaccaaaactaacaccaaaccccttactgctttgtagtttccattaaaccttaggttatcctgttagaggtaactgtttaaaagcctttaacaatagggaaatacatttgccttcttataattaggtagaacacagtggaaggatacttagcatggcaggcccacaggaacacagctcgataggaacacagtgatgatgcagagaaaatgaaatacgccacagaaaaaaagtaagagaggaaattttttcattgaaagttgcagagtatggttaagtggcagtttggccagataattattttctgctttgatgcagaattgtttatgtctttcatgttatgcctcataagatacactatttcacatcaattttatattcagccaaggaccaatttaaccaaaccaaattcagctgtattgaaaaggggagcaagttctttctttaagtggaggggatcttttctggccatgtaatgttcttttgactaatctggcatgctaattttatattgatttctcttaagtatgaattagtaagtgagggtgattagattatctttaaggttacaattccagtttctagacctttacccctggttattagaccttatactacagtaacttagccaaaaatatgttgtgttgaaaaattagtttggtcctgagtcttctggtttcatgattcctatacttttgcttgcaaattaagagaattgcacattcatttttataattagtaactaagacggaaattaattctatctagctttatctatctagtttttttatttagccttaggtgttctctctcttttaaagaaatctatctgtattatttagcttgttcttttagcttcttactttaatgtttttataaaactgttctgtgtttggtttttgttgtttgtttgttaagataaatgaccattaaggcctggataacttctgtttatcagttccccacccacttttatagtaaagtatgaatagtgactagaaaggaagataggccttttcctacaaactaaagtgactaaaaataacttttttgtgatttgtttgtttgtttgctttttttttttttttttttgagatagggtctgaattaagtaagtagccttggctagcctggaagtccttatgtagaactgtcttgttttgaactcataggcatttgcctctctgtctccgcctcggaggactgggatcaaagccaacaccattacattagcccctacaaataatgaattacaatgccttttaggattgccttctccaaagaacacttgtgtctaatatctaaataactgattacttcagtttgttcacactatattggtgatgttagttttaatcaatagtgagtgcttccttttgctttaaagttgataacatctcagtacaagtatttttgttttcatatcaccagatagcccagaagaagcaggtgatgtaagagggggttcgtttgcctcattgtttgagaaggctttctggctgtggagggtgaggagggagggcagaggccctccatccctccatccatggcttcctgagtttggggcagagttcatggtggaaggagtggaggcagtaactcctcatggctgtgaaaggaagcaggaaccagagaaacctgcagataatttgccctgactgactgatttctgtcagccagatttcacttccaaaggctccacaaccgggctgggaggaggatttcagattgaaactataacatgtttgctttcttattaataaactttttttttgcagttaatttttctttgctttgtctagtagggtatccaaaagatattttaaactatttttcttttatctaattccatatttttcttatatttcccccaaacaagaaatatataaaacttaaatctttgctattgtactatagcgttacctggagtgaggtaaacttaaattgcaaatattttcatgcttaaattgtcctatgcagaaaggtttggaagatatagtgtgctatgataagtaaaatttcatagctgagatctacaaaatttgagaaaaatatttagcttcttcatttgagatctgggattcatgtaaaaaatggaaatggaggaacactgggcttggaatttgctttctaattacagatgatttgctttttttaatctaaggagtgagctgacagggcaggcagggcaggagggactgcggaggctcccacagtagttgttgtcgttcctcttaactgcttccttctttttttttttcctcattttttgcaatgaagagggggtgatgcaccaccagcacttggcttgatttgtgcttttccagtgtcctttaggctggtgggaaagctctgtggctttaaatctaagcagtcgtgaacagactggggcatagtgtgtctaaggagcttgtcttgtaatctcttctgcccaatgactcaggaaaatgtagatgtaacagaggcaaactactttttacacaaaatgtttagtacagtgccaccaggtagacctttccccacctttatgttttctcttggtttccttttataatttgctaactctcatcttcctagacagtgtttcatggagctcaatttggaaaatgttgaaaatgaaaaagttatgtgtacagaattgttctaagtttgatatgttttgaggtggaggcatgctatatgacccaggctgacttatgtatagtccatgctggtttctaattcatgatttttacactcctgcctatgggtgtccatcaagcccagtttgtctatagcttttataaggaaatgattagagctcagtcttgtgacataatgatttaacaaaaagctattactgttttgtaaacccatgtataacctgttacaggttcaagagtcttattgtttcttgactgctggcatatatattttctttctgaagaaactttgcaaatgttcaattcttttttttttttttttttttttttttggtttttttgagacagggtttctctgtgtagctttgtttgtactggaactcactctgtagaccaggccggccttgaactcagaaatccgcctgcctctgcctcccaagtgctgggattaaaggcgtgcaccaactctgcacagccctattcattacttaatggccctctgaataatgttttcaagtcttatttttaattgatagtcaataatgatgctagaataacaaatagttctttctcattgaaaatcaagaaaaaaaaatgttgagcggtggtggtgcacgcctttaatcccagcacttgggaggcagaggcaggcggatttctgagttcaaggctggcctggtctacagagtgagttccaggacagccagggctacacagaaaaaccctgtcttggaaaacaaaaaaaaaaaaaaggaaataattaataataataataataaacaggaaagattacttctgtttttccattctaacatttttttgcccctttcctcattttgccttcctcttttccattttaaaaaaattagtgagttaacataatagtcactgttttaaactgtaatttgagtgacatttaatattttcacagtatagccagaggtaatgcagtcaccaccgataccaaattccagaacctttttatcactcctgaaagctcctggctgcccttcccttctgctggctgtctttcctgcacatggggagatttagtgtgttggacaataataatgttatgggcacaaactagctttgctatttaatagcggatactgttttctgtacatttatttatttactatttatatctgttaactattgtataacgagcttgcatataccatcttactaagtttgtagacggaatctaatccctatttcacttttataagtgccttaaaagaaattagaatcttcagttgccacagcttagaagtagcaagaagctggaagttgagtccgtgtctgtgagattccaaagttgattttttttcttttttgtttgttactgtatttaaggcaaggtctcactgtgtagctaaggccagtcttgaactcaaggtcctcctggttccatattctcagagtgctctgtgtataatcttatggtgatctggccagcaaaaattcaatttttaaaaagttttttaaccaggggagggatgcagtctggagtttgaactcagtcattactaggtacttcaccactgggcattacttaaagctgcttcccttaatatcttttttctgtacatcaaaggacaaaatctagaaacacttggacaaatcgagactatacttaaaaatcatgaagcctcgtgcaaaaccctgtatttctagcttatttttaaaagctgaaagagctgtcaaaactgaattcaacattcctgtgtgatggatcaagtatggttgtgtaatgctctctggcattaaattgttaccatttctccattaaggactgtttcttagtgaggtttccattgctggaaatgtgcctggcccaaggaatggcactattaggaggtgtggtcttgttgagggatgtgtgtgtgtcattttggggttaggctttgagaccctctcataactgcctgaggacagtctgctcctggtgtcctttggatgaagatatagaatcctggacaccaccatgcttcttgctgtgataactttcattatattgaaagttgttatattttaataatactgttaatgttttaccttgcctagtttattcaacttcattgataagtgtgtatatggaaaatgtacatacagtttgtcgctgtccaaagtttcaggcagccatatggaaggcgtgtgtatagtgtaaggtttcagctgaggatacattacattccttgttaaaccttagtgctgcagatgttctctcacttccacttgaagggctgtctatagcatttcttttaagttttcgtaacttttgtttctctgggaatatttcaatttatttctcattttttgaaggacagttttgctggatattttttatttctaggaccttaaatgttatattcttacttcctgttctgagggggaaaaatctgctgataccaacgctcctttgtgacatgttccttctctctgtatttgagactgtgggtctttctcaggagagggtcttgcataatacaggctagcctggagtccttgtatatatgcaaggatgtactaactgacttttgatcctcctgcttccacttgactagtgctctgatttcaggagtgcaccaccatcaaaggtttatgtagagatggggacagatttctcagagctctgtttgtgttatccaatgagatatgcccccagcctgtctttgtcttgtgatggtttgattgcgatctgtcttagtaagtctctgagtttttgtgaattgtggttcattgagccagaagttctttatattcttttatcaaatttgagaagttttgactgtacttctgtatataattatttttctggaactttaaagatccctaaggtagtccagttgctgttgtctcacaggttggttaatctctttacgtttcttcagtaagttgctttctgtattttgatatttatcctcctaccctagttcctgggtgtggaatgtggtttactccaggaaagaagattgactttctcactcttggtagccaatagctccttagccggggatgggactttggtcatcacttttctttgcttggcttgtgctttcacaggtcttgtgtatgctgttagggttgctgtgagttcatatgtgcatctggcctgttgtgtctagcaagcgctgtctccttgaagtcacctatcattcttgctcttacagccttcctgccctcttccacatagatgcctgagccttgaaaggaagggtatgatgcagaataccatttgctctgaacattttgaagtctttctttgcaggttgtatgtactaattgccatcaacagaagcttctctgatgagggttgagctgtgcactgtctgtggtttatttagcagtagtcattagcaatcattctattgctgtgtccacttacccgaggaatattggtaggttttccctaggctccatgcccatctagctacaggcttttggcctcattttgacaatgttagatgtggcttccatcttatagaacagacctaaatctaatcaaaaggtggttggttattcctataaacatttttattccacttgactgtacttttcagccatggctaggattacaagtatgaactactgtattgttctatttaaatttttattaagagttttttcatatatcttgatgatattctttcccattcttcaactcctcccaggtcttttcccacctcccattccgacaaatgtcatgttctttttttctttccttctcaagaagaaaaaaaagaaaatcaacaaaacccaataagacaaaaagtgacaaaacaaaacagaaaagcacaaaaaccatggagtccattctatgttggccaactactcctgtgcatgagcgctgattggagcgtagttgatatgttggagaaaactgatcttctgtttctcagtaggaatcaactgcaaatcgtttcttggttagaggcagggctttgtgtctgcttcagatttagtgctgagattttgtttggtttgacttgagcagatcttgcacatgctgaaacaatctgtgagtttgtgtgacacccttgttgtgtctggaagatgctgtttgcttagactcatttactacctctagctcttcccatctttcttccctttcctcggagtagatccctgaaccttgaggggaggggttcaataaatgcatcccatttagtactgagtgttccaaagcctctccatttgtacgctgtgttgatgtatatgcttaatttcatgtgggggttactgctttagatcattcagtttccagataaaaaacacaaacttttaaaaattatttataagccttaatgagcactaaagctgggctggtatctaccttctaggctattagtatctacttccttattggtagccctgagttataacttgccatatttcatctgggccactcttaactccaattggccagccttcatgaccgagttttcatgaatcacttaaccccactgtggcttctcctctctctattgtttcctgatcttctgcctcagaccccaagcctgggaacccaaaccccacctaactctcttcagcctagctataagctgtaggcatcttcattcaccaatcaaggatagctttcagggttatagagcattatttgatgtatgtgaggatcaccttggcccagaggtaaccagggccaatatttagcattacaatatataacaacagaccaaaccttaacggttttaaattaaggtgtaaggtttatacagcaaaggctggtaaatgtgaaattcacttgtaggtctaaatcttttagtacagaattcagcattgctatacatagcaacagaccaaacctcaacacactcttcagttgtgggtctctgtgttaatcaccatctactgcaaaaagaattttctctgatgagtgacacactcatctatagggagagcagtatgttaggaatatttctgtttctttaatagaataatagtagtagtaggttttcccctaggctcatgacttgtctagccttaaattcttagcctcactagcagtggcaggcatgggttctattttaaggaatgggtcttaaattcagtttttaaaaagtggttgtttgttcccataacatttatgccaatattggatcaatatatatgcccgcgagcatgcaggtctttgttgtgggtcacagagtttgtagctgggttatattgatgactacttttatcttccagtcgtgtgcaaaagtaccttccagcaccacgagtgctagtcagtagtgctgaatctctagttggctgtcagctcaatctctctgtgctcgatgacacaagtaagcagtatcttaagcaacaggactaccatctggttgtggaggaaaacagtagccttggcagtagccatgatgttgggattgcaagtatgtgctatcgcactttgttctttttttcaagacagggtttctctgtataccccttgcttcctggaactcactctgtagaccagaaatccacctgcctctgcctccgaagtgctaggattaaaggcgtgtggcaccactgcctggcctgtgctttgttctttatgtgggttctgggaccctaaacttagactaaggtgccttcctagtcctggaattttcctttttaaaatttttttatttgtttttgtatgttggggtgtgtgtgtgctatgccatgccacacttttagaggtcagaggacaacttataattctttccttttactgcatggttcagtttggtggcagttatcttttttttatcttctcagctacccatcttgttaataactcagaagctgcactttcctgcctcagccttccgaatgctggcggacaagtgtgtaccactacacctagctctttgtttctctttcactttattgatacttctgttcatcatttttcttgatcttacccgtgtcttttttttctttttttgagacagggtttctctgtgtagccctggctgtcctggaactcactctgtagatcaggctggccttgaactcagaaatccgcctgcttctgcctcccaagtactgggattaaaggcgtgcgccaccacgcctggcatacccctgtctttcattagctttctgagtatcattaagaccacacaaatctttgcctagtaaatttgctttctggtttttctgagagacagtttgttgactttttaacttattcgatttttgagtatccacacatcttattttggggtgtgagtatgcacacttgtgtatgcatgtatgtttttgtcttggtcttgtaaatgtgtgtgtatgtgtgtgtgtgtgtgtgtgtgtgtgtgtgcatgtggtgtgtgtgtgtgtgtgtgtgtgcatgtgcgcttgtgtgtgtgtgtgcatgtgtgtgtgtgtgcatgtgtgtgtgtgtgtgtatgtgtgtgtgtgtgtgtgtgcatgtgtgtgtgtgtgtatggggggaggtagctaaaaacaatctggatcttgtagggtcgaagatcctctctcttttcttgatggcccagctttcccttgttttctgtattgggtatctactatgctatacctatgcaaagattaccatgctaaactcatgcaaacttaagatctttggggctggagcagtggccgagtgtttggggacactggctgttctcacaactgcctgccaatctagtctgagggtacccgatagcctcttctaaactcgggtggcaggcactacatgctagtggcatgcaagtggtgcacagacatacattcaggcaaaatactaaatacacaaaatcataataaattaaagatcttttaggcttgggctttttttctaggtatagggaatgacttcctaaattttttttgtatgtgtaattaatctcagttgttattatctttaaatgttgggttctttgaaagatccaaaggaaggaaaaagaagtgggcagggcgagtagatttaaaatcccttgatgtcttcagttggtgggcgacagcttcctccatctacgtatgcgtgttcaaaagcagcaattagtgaccagcacacagatttaaaatattggaacgtacggcatttattattaactttggcttttgaaagttgtttgtaagtctctatagaggtatatcaatgactgtatgagaagtccttgttgtataagagctaaaatcagggctgtggagatggctcccttagtaaagttcttgctattctgagttcccattgttctttttttttttttaatagaagaaaaggtttattttactcacagttccatataacagttcattatcaaaagtaatgaggacaggaactgaaacagggcaggaacctggaggcaggagccaatgcagagagcatgaaggggcactcctgactggcttgctcagcatgctttcttttcttttctcttcttttcttttcttttcttttcttttcttttcttttcttttcttttaatattttttattattacgtattttcctcaattacatttagaatgctatctcaaaaatcccccataccctccccccaccaccacttccctacccacccattcccatttttttggccctggcgttcccctgtactggggcatataaagtttgcgtgtccaatgggcctctctttccagtgatggctgactaggccatcttttgatacatatgcagctagagtcaagagctccagggtactggttagttcataatgttgcacctacagggttgcagatccctttagctccttggatactttctctggctcctccattgggggccctgtgctccatccagtagctgactgtgagcatccacttctgtgtttgctaggccccggcctagtctcactgagttcccattcttagaactcatacaaaagccagcttgctcagcatgtttctgcgacctctgtgacaggaagcaggcagagaagactatcctgggggcttgctggccagttagcttagccaaaataactagctccgtgttcagtgagggaaccgttctcaaaaacagactagttgcaaagtcatagagaaatacctgttgttcccatgacacacacacacacacacacacacacacacacacctaaaattgtttaagttaaccttcattttctgtcagagctgactcactgaaagtgtcagcgtttgcctagattccctgggaaaggttccgcaagtgcagtcggtggtcagggctggcttctggggctgcttctctgtcctcttgaactactttggtttctttgtttctgttttgtggggttttttaagatttgtttttgttgttttgttgtggatttttggtaatactttctagcatttgaaatacatgtttatataaaataaatttaaaattcactattgtggcttatctagatttatttcctaagaaatctttcatgctcatacatcagcctcagtttatctcagtgagacagacacacagacacagcacagttggaaaggaggctcaacagggataggagggtgagagtggtgaggcgatgtgagacagacacacagacacaccacagttggaaaggaggctcaacagggataggagggtgagagtggtgagggcgatgtgagacagacacacagacacaccacagttggaaaggaggctcaacagggataggagggtgagagtggtgagggcgatgtgcagtcagttccttcaaggaagatgcagttctaggaggtgtcttaggtcgtgcagggttagggagcattgcctctcactgctgtctaatattttagcctctactatctaaatacatctctgtaggcaagtttgcccatttctctttggaatgtgctgtttcacttgtctttcctcacttgtctttctatgtgtcagactgagagaacagtgggggaagtgcggaatgtgtccctaagtaatcagttctctttgagacagttatccccccaccccttcaaatgatggaatgatgtactgtacccattaaagggctgctttcttctgttagacttgctgttgctcacatgctagctaagaaatcagaatgttcaactgttaaggggcacacagataggatttccctaagcctaaggtaaacacacggtaggaaagactcttgaaagaattatgagtttttagttgcaaatgacataaaatgtctttaccagaaaggaataatgctctggaggaagttcccattgtggaaagcagaagtttaggaaacgtggtgtaggggctacagtctgcttagacaccaatgcatggtcctacatcctggttgctgtctgtgaattcccaggtcttccagtgagatctttgaagaatctactgttctcttgtaccttgctgcccactctgtaggagtgagtgtctcacaacaagggaaagagaaaagaaatacctgcctctgatctcagtgtttgctaactggttgacataagggtggcacaatttccttatgaaatttttatacttcatccccctttcagaaatttgtagctgtgtttacatataagaagccgtggtctttgtttgtttgtttgggtgttcttggactttctagcttccaaagcttcggacagttaacttctgtggggcattgtgtgcatacgtggtgtttactttgtgttgactttcttttcaactgagttttcttttaaattgtttaaactgctttgattccttttgtagacacagctttataatgctttataagtcctttctttatgcctttataatatagcctttataaatcccttctgtgcccttagattcagataaatgttgactaaagaaattgatgggttatattttgctcagaataactgattgctaactctgctttattgttgtatataattactatattttctattgctagctcttaaataatcaagaagcagctttgcttaaattatcaagtagaaaagatttaacttatgaggaattgttaatatatctcctactactgactcggcatttttcttttggacagagaatagagaagtgaaaggtttagggctccctgcctttttcctgtttccagcattatacaccagtcaagcgtatggaattctagtttctttttgttctgttgctccactccaacctttagttgatactgtttttgtgttccttcttatacaccactttgtgctgttctgatttcatctctgagcactccttctgccattgtgatgaccgtgttttaaaatggagctttgtgagctctctgcagctaagtgttttttcctgaataatttgttcattacaaaagagaattctagagaatcctaccaagtccatagcattgttactgtgattgctgttttgagatggtgtccaactctaatcccagctgacttcaaactcagttctatagacctggctgtgtttacatgtgtgcggtggtaacatgcatggcacatgtcacttagtgggcttgacctttctttctctctctttctttctttctttctttctttctttctttctttctttctttctttctttctttctttctttctttctttctttgaatcatcaaagtatgacttcatgttttgtcttttaaaaaattacatttccctctgtgtttaaacaaatgagcctagtttatagttccccatggattacagttaaatcctctctgtagtcttcttttagattgggttgtagattcctaggctgctgctgaggcgaagcatttgcaatgctttacagtccagtatggtatctcactatgccagcatttccttccttgtctgatgtcagctctagaattacatgaacactttccctctgtttcctgacatttccagagttgtagtttccttctaaaaattatttataaaagagaactaaccaaccatttcaagatttttttttttaaagaaaaacctcagaagttaaaagaaccagattcctaatattttgctctatttttcttgtaattttataatgtattccgaggatgtgcccactttggtaacctgactgtgacacaaatgtattgtgtcatactgcttggttttctttctttaattgaaaataaaaaatagatattttttcatacaatattctgattatggtttctcctatcccaactcctcctagtttccctcccttctcccatacagatttacaccctttctgtctctcattagaaaacaggtgtctaaaaactaatagagtgaaataaagtaagcaaacaaactggaataggacaaaacaaacaaacaagaaaaacacaagacccacgtaggctcagagacacgtgtttgcacacatagaactcttataaaatcacaactggaaaccgtactatgtgtccaggagatctatgttctcggttttaatttacacgcacacacacacacacacacacacacaccctgctctgtaaatctcacagtgattgagcacatttggtgctcatcagtttctcgtactcctgggtcttcctgaccgacctaactctgacctaattgccttctgtgtgtgcagcctgaggtaccccttgcgatccttggggtcctcacttcttttacaggttgggctccctggttcccagaaccgattatgatttttcactctcaacatcttttacaactgagatagtgtatgggaaacaaatgacttgttgtagaacagtgcctttattgtattatatactcacccacgatttatagtctgtcttgtatagcattctaggctggaagtaaattttctgaaaaatcaaactttgtataattgtttttaggaagctagtgttaatggcagtgcgtttgtcgttttgtcttatgctgtctactttccatgccaactttagggtctggtgttctctttggcacttagaaataacgtagatatatatggctccatttgcggctcccctagaccccctttttaaagtcaattttattagctatttatgtcttcatcttgggaactcatgttggacctggagatgtaaactgacagaatgttttgctgaggctctaggtttaattgccagcactgcataaacccaggttggtgatacagacctgtagtcccagcaccccagaaatggagggaggagggtcaggaattcagggccagcccgggctacatgaaactattttctccttttgtctcattattaattcttcaccattataccttgctgagtcttctgtttcaggcctgaaggttaaacaaatttacatacataaagtacttaaataatacctggcatgtaataggtgctttggtacctgtgatcactgtgtggtttcacagctggttggaaggagtggcccctgctctgactcttcatttactagcttcacaccttggacaagcttcataatctcttgaggtttacttccttttcctgtaaaatgtaaattccatctctgcgatgttggtcagggacaagagaaagtatacatgtatacatgtgaaaaatgcttacagaactacattgctattgtacttttcagattgtgggtttttttttttttccctgctaggaagattacattttaagcttttttttttttttcatggaagtctgtgagctgggtacacttgaactgctaatatcgttttgtcaagacgtgattgtaatttattagactgaagacatagatatgaaaacagtttttgataaagtcagctctacttcagaatgtataaatctgtgtaatgtaataactattaatgaatgaggggatatgtatttgtgttattaatagtatgtgagataagggtaaataaatctgttttagtcctgtgcagcattaatgtaatttgaaatattagctcatttttgttaatggtgttttttttgtttgttttgttttaaggtttttggattcaaagcataaaaaccattacaagatatacaatctgtaagtatgctttttttatttgtctctgttaaaataactaaataaaagttatttctttgttgaagataaaaatatatttagatatttttatatttgaggaactggattcctgaaaacagttgcagtctgatagagagagttgttgggtctcgaagcgtggtgatgaggtgcagcagcttggcacagcctccggttacttgatctgcttttacagacttggcacctcgcccatccttgagcccataatcatgtgataatttgaaatgtaatccacagcggagctgctgttagtattaacgatggcttctaaggagacagactccagggtggatggacagacttttgtttcctctgtgcttgttgatcaatatactgaaacagctatttgaatattttctgtgtataacctagtaagttatgcagcattgtttagttatctagtataggatttgagggattgctcattaaaacttattggcctatctttaaaccttcactttcttttgacttttggagtagtgacatgaaaacaggaaaggaagacaaatcattaaacaccctttgtctttcaaaaccatttttattttccccaaatactgagcatttttaaaaatttaaaagataaattaccatgtttctattatgtcctttaattttctatgtctatgatttatataacaggagaatgttatgcaatggtagaataccaattagtaattaaccattttctgtagactttatcaaatataactacaagtgttttctgttctgcttcgagtggctatttgaattgctacccagaaggatggagaattttctatgtcttgttatagtgctagatgttacttttattttttcagtctttaatgatatttctgttttgataagacttcaaagtattcatgtgcaatagttaccaatattatttctcttcgcttttgctgacttcagatcagaaaggtgcagccatggtgaaacatgcagatagagtgctcatatggctagttccagccctctagtagcctatagcttgatgtgaaagtaggagggagcaggagagaagtgtggacaaagtaactggccccacaggaggcctctgtaaaagaccagatgtgtgggctgtgattaacttctgataccttctttcttctatccctgcttgttatatacttgtaagactaagaggagtttctgttttatttcttttaattttaagattatttctttgcaaacataaatttaaagatcttgaaatatttccatggcttttctactaatgaaaatcaataggagttatctattagacctgggaggatgagccaaggcaagtcagaagattgatagtataatggtatttgaaatatggcagataactcattttgggcaggtggtggtgtatgctggcttaggtggggttgtgctaataaaaggtggataggagaaccacaagactgtttctgaacagctgcattcagaaggtgactgaaaaaggacaagaactgttgaaagctggaattgatgaaatgaatcatttcagactcacactgtcaggtttggggatttagagaggtcccaatagggaagtagaaagacattagaagacacatttctgcctgagctgaaatcttatgcctgtttaacatatctaaagcacagggaggaaattcttttcattcctgcctatagtgacttcctgcccctagaatttagggattaggtttatgctgtctcctttgttgtatttcagtatagttagaggtggcattgggtggacctaggaacttgatttgagtttccaagcatttgattcccaatttaatgaaccatctctttattagttgagagcagcctttagtgcatatgaacttattcccttgtcatttggaactgaggctttcagaatggcaaaggatctgaagggtccttttagcagtgccttcttatcttatagacagggcattaggcctaggaagttaaatgaggtagccaaagacaggtaggtgcataataacagactacccactgtttgcaccagaatcccttttgtttgctggttagctcttcgttttatttacttcaaaagtttttaaacatatacaaaattgagtgttttaatttgagtaccccctctcccctgcctactgtgtatctgattttaggcaagtgagactagccacaacagatgtttttgttttatttctttttgtcttaggataggaattacaggtagtatgtattttttttttcttggaaatgtagatgtttgaaggtcctaaagtatttttcactggacatctgtatagttagtagtttgtgagaccttttatagcagcagtgttgcacatgaatgaagaactatcagcctaagctttctgataatctagcttatctattattattaatcagttattttgaaaaagggcaacattaattaatcagttttatatgagtgtttttaaaatttctttgttgctccctgttcagagaatacaagattttaagtttttattatattttagtgaatatttgctgtacttggcaaacatttaactgtgttatttttctgttaagatttcctttgtaaaacactgtagagtgaagaagagagctccctaccatgtagttctatggcaaggcctagttgtctgcaagtttgcctttctggtttcactcctcctcttaatttctgttgccaccttgggaaacctcattttccttgttttttttttttttcatttctcttttcatataagccaatttaagataaggacaaaaatatcgtttgagttttaattacaaagaaaaatttaaatccaaattgttatttgctatcttctattttagtatgtggagtgacttactgctaatatgccataagaaatttaaaagaaactccgctgtgaattttggctatataccagagattctaactaaggtggaaggtttcttcttgaccctgtgaccctttctttctcttgagcactgtttcacaggcagccctagcatgtcctcccaaagcccctccgcttgcctataaggagctgcatgctcccctccccccccaagtcaattgttaggtctgtcttcagtgacaaatactgctcatgtttgtgctgtaaaatttgtcactgctttttcatttaagacttgaatgtttctgttatgttgaatgaaactgtaatagaagttgttggatttagttgagcaaggatactaagcttgagttcctgtctcacggtgacttcatgttgttattaggaaagcttttaagggcctttctaaatcttagcttttccatatatacatatgcctcacatatacaatggggatgtaaactgttacatgattgtgagggtgaaaacatggatgtcagctgtaaggtgcccatatcctgtagacttcagttgttactgtgttcctttcaccttaactgatgatacatgacaaccagtttgtaatggtgatcttaagcagtgcttattaaaccaaacttttcagagtgtttgttccatctttctctggggtgggaccctcccttcccctcctctccccttccctgcatcacctccgcaggcaattgggatccctgaccctagaccagaaagtgtggcaaactgaaaaatctgacttgtaggacactaacaaccggcttcttagggtatgtgcctagcttcctcttgtttcctgattgtatccttaattcttgactgtcttccactgtgggctcttcaccacacagcacctctcagaagagcagaacctggcttccctgtgtggagttctaacacttggaggtggagggagaagggaattcagagccagtcttgggtatatgagatcctgactcaaggaaaaccaaagaggaagggaggaaagagaatatagaatatgtgatcttttgtatatgtgtcagttttcttcttcctatctcatttttaggtaagcagacatttagcagagtatttagcaaggatgcatacgtcatctaataaattttctcttttcaaaaacagtacatcaggtaatacactaaaagaaaaacacatgtgtgtgtccgtgtctgtgtgtgtgtgtgtgtgtgtgtgtgtgtgtgaatacagaagttaattcccctcaggtctgctccattgggctgtagtttatggataatttgttcaatctttgtgtgaactgggttttgaaatacagttgagttgtacaaattccagatgcccagtgcaggcccacagctatttatttggaagtcttggatcagttttattttggtacatagaaaatttcagttttcaaaaaactaaaaaactaaataaaacaagaaaatccatatcttttgtgttactctagtatccactgtggtagactagtcggtactcagcaggtatgttggttgaacaacctcagattgggtcctgttcgagttgagattacctatttataactttggagtttgagatttgggctaaggaataatggaactttgttttaaaacactaacttttatttttcagtaatttctttttgtttgtttgtttgttttttttgagacagggtcttgtatcccaggctggcctaggactcactagatagcaaaggctaatcttaaagatataatctttcccagtaactcttctgaagtgctaggattacagcctgtggtaacactcctagcttatttgaataatgcttaagtgtctgatttccttagtagttggagtcaccaggatgcttctgaccccactaatatgtaggatacccttcatagtatcactgattagtgttattattgaaaagctaagtgtttgtcttaatgtgtcagtattttactatcagtgggttttagttattttattgtgatctggtattaaattttgtactctgagagattattggaaatgagatttgtatataaaagagtaaaggtctggcttacaatttttagtaagcattgtgttaataattaaattagtatcattcagttgtcttttacatttcctttgttctttttctttatttttaacatgtatgttttaagtaatggtttaagattgtatgtgatcatctgtcaggtaaagataatagtaagagtagctatttattcataggtatttgtgaaataaaaaatacattctaaagccatgtatagtctttatccaagaaattacagggtcagtgcagttgaatttacagtgttgcatgttgatgtcacaaattctgtgaacaaatatatgcacacaaattgcatgcatgcgtttaacttttattaaagctttggtctccttaattataagaatgataatagtacctacttcagaattcttgaagttaacggaaatagtgactgtaaaaacacttagcgcagtgtttttacatgatagaaaaggtggtatgatagaaagggtggataaatattgctaatattgatactcttccttccagtgtgaaaggtaactttatgccacatttaaactttcttgtagatgtgctgagagacattatgacaccgccaaatttaactgcagaggtatgtataaacataaccacagcatactgtataactaaagaccaatagacttgtcttttactgcctggtgataattatcaagattagtgagataaaaatcttaagaatggcctttgacaattaaaaaaagtgtatttaatgttagagttgttctttaagacctatctattgtcaggaaaactaaatcacagaatacttggagaggtcccaagactaaactaggattggaggtgcttattgacggtgtgggacagctagcgctgctggaaacaatcacaagaagagagcagaaccattttaacttttctacatcgaagaatggcataaagttaggaaaagatgtagcattggtctgtctgtctgtctgtctgcctgtctgtcttctcagaatcatgaagcactaaggagtaagtaagaacagtttctggggaccgacagacctaggctactgctcattaggaaacatgccatggttgaaggtcacttagctttaaatgtacattttaacagactcttgaatgttcttgtgtgccactggggaaatgaggtcgggagcacagttagacagatggttaagtaaaagctggcctgcagcctcttggtgaatgtagtttgccattgtttaccacagagctttcctgtcatggaaaggagtaaatggatggattgttcttgtaccattttacgatggcttgctttaggataagtcagagtttttacatattagataatatggcagataatcagaacagtaatatcaccaggattttttgttttaattttaagacaagggtctcagggtctcagtgtcccagagtgaccctgaactcaatgtttagctgagggtgactttgaacttgtgatcccaattctcctgcttttactcctcaagtattaggattacagacttgcaccacatcctcagttgtgtgtttactcaaggcagggatgagcccagagctgagcatcctaagcaagcactctgcgaactgagctacatcccagagttcataccaggatttaaggatctcaataggatagaatcaaaacagatactagtaagataaaaaccagtagtgatagaacggaagtcttgcttctagataatagcatcttgccttcaaaaacttaactctgactatagagaacaaagacatcttagattcttaattcatgtgaaaaaaatctgaaacttaatttgctataaactttacttcagttgtatgtttttctgtgagtgattaatctcatgtatatggaaatataatgtttgtgagaccattttaaaaacaagtcactgggtaattttattatgggataggaaaagtcagtcttttccatagttgactctattagtaattatactttcttcggagcatgtctggcaatgctgtagtaatatctgctattggtcctgatagaagttactacttgacaagaggcctgggtgacgtgcatttggattcagttgtactgataggctatgacgtgttcccttcatgcacagattcatcctccctggagtgaagagcacaatgcttgtttccatgtctaatgaatgcatttaagaattaataaaagactttctttaaaatctaggtttaattagtaataaattaaaatttcctgaaagttaggcttcttttaagaaccagtaagtttatatataacattttgaaagttaacctatgtttttaaataaaaaatttaaaattttcttacactgggattatctttttgcaacagttgcacagtatccttttgaagaccataacccaccacagctagaacttatcaaacccttctgtgaagatcttgaccaatggctaagtgaagatgacaatcatgttgcagcaattcactgtaaagctggaaagggacggactggtgtaatgatttgtgcatatttattgcatcggggcaaatttttaaaggcacaagaggccctagatttttatggggaagtaaggaccagagacaaaaaggtaagctgtttactttttccttcctccctctttgtggaccaagaatttattgggaaacaggttttctccctcttgctttattgaggtataaccaacaaagtcttaatctacttacagtgtgatgctttgagaactgttatattgtggttgtatccacttagtgtatccctcatccctggtatccccaccctcttccttagctgtactgagaacatccaagacctacctggagtaggtgctaggcacacagtatggattttgatgacaacttgaatgccattacctagtaaagcaaggtatttaatttgatggtaaataaaacattttctgatgggggtattcactagtatagttaactaatcaaagattcattggttattcagaaaactaaagactgttgaattagtggcatgttttgtctatggtacaattgaaaacaaaagcaaattcttggactgctttttcagaggactcgtttagttagtgtaacaccaagattctttgcatgtttttctttctccaagcacagcacctatagtacttcagatgaattgaaagctcagggtagcagtgaaagtgccccaacataaggtcataaactcacttaacctttgagttggtttgcagtcttttttgtagacattgtaagtgacaacatcagtttgcaatgccaagggttggacatggctgctctggggagtaagacatttgaaacttgattctagtattaaatttggacttgtgccccacccccgcttctcttctgcctcctctcccttctgtctttctcctcctctactccattcttcccccttctcctttttttgagccctgattttatctggatcaactttgggccatgcccatcacactaaggtctgtggctgcagcggtcctgggccctgtacttctctttcacctgctttttaaaaaccctgtcgttataactcttttgagtttgtacaagaatatcaagactgtttgttcattggtgggagttcacaaaattacatctttaatgcagtaaaaaagtcatgtgttagaaaatcagatttaagctagagactcctcaactctgactcccgatgaagtgttcagatgttctgttattcgatgtatgtggtatatacataaccataaattgttgttggtagcttccatttgccttcagacaaaatataaaggaacttctaacaaattatgtctcatttctcccatttaaaaaatcagtaccccttacctgagaacagtaggtatctaaatgggttgattctgttcaatagtgaaatttatgataaacaagttttaaaaacaagttgaaagcttgccattgtttgactcttacatcatccttgctctcagtgttatttttattcttgtttagtgaaaataaattatgaaaactcttatttcacctatgagagaaatatggaacataatatgtttttgaccaattaaagtaggctgtgtcagataaaatctctaagactagatacgatcatctattagtttctttgccttcaagatcattatctctgtggggcaggaaaagattatggaccattttaattttcaggttaaagcattaaactgcttgacagcacagcgttgtctggcttctagatatcagtggacctgtgtgcagtgaagagctttctaggtccttctgtctgttgacaaagcctcttgagattctcttgcttgaaggtcttggctctctcttgtgaatttgggtctttcacagcagtaggtttttcatgacaaatcatcagcaggggaacttttcacaactgtagaagagagtctcctaggattaatgtgatcttgctacccagtgcagcagtgagggccagggcagagatatcatccacttttaccagtgcagggaaaccagaggagctgcagggaaagctgctagagtgtggaggactcttagggccagcgatgtgctcagccatttttatttaacttttttacattaagacttttcagttctttgagactgtaataaattatatcatgagtatgtatgtatgtgtagtacaacctgtggatatgtttttagggcttaccatttggataacaaattggcatgctctcttagcatttcttagttaccagtagatctttgtctagggttgaggcttggtcagctttctcccattctggcatgtcttttgttctccttgctcagctcatgtttggtagtcatgttgatgagactttatgggtgtagcttctgacattgccagtagacaatctcacagcaaactcccagatcctctacctcttacagtctctaagccttcgtctacaatggtccctgagctgtaggtgcagttattgtgttgtagatggctcagttggaactgagttccacaactctgtgttttgaactgttgtggttttctgcaatgttttccttatgctacaaagagaaatttccttgatgagggataagcactatgcttatctattggtataaatttagagcatagttatggactatatatattatttagtaaattagtggttgtaggttctctgtcaagaagcatgacttccctagcccaaggtagttggctaggtttccaataccaggcagtttctctgtctttaaggggatttaaaatttttatttgtttatctgtccatccatctgtccatctgtctgaaatctatctatccgtccacccatttaggtgaggcctgagagacagagtcaaggaaatgtcaccatctttaaaaaaaaaaattaagaaggaccctttctttaaaagaccttaaattatttgctgattatttattcatttgtttttattgccatgcataattttgatggaaactttgaactgattacactaagagcttaattactagtaagtggtactcctgatacttctatggcctgtcttgtattttgagagagcaatgtttgctttggagaaatggatgagaatatttgaagtcctgcgactctctcagccatttcctgccaggaatctcagctacttgacatttttttctgtttctgtttgtttgtttattgttgttttgatgttttcatttttctctctttctctcattccccttttgtaatggtaaggattgagcctagggccttgtacatggacacatattctaccagtaagctatatccctcagcccttgtttgactcattcatcatgttaaactacatgaccttttgttttgctttgtttctgtttgtttgctagttcagacaaggattttcacttgagacaaataatggttgtgaaatggtttaggcttctctaagctctgctgtcaatctccattctttttccttcacttcttcagaattgccagcgtgtgtatagagtgataagatgctggggtggtctgtttccttatgttcaagggtaagagcaagacttgggcacttgaactttcctgtaaatttctctgtttccaggccagggtaaatgcaggaatccaggttagacgcaggagtcaagttaggtgcaggagacccatgtcagaaactgaggtcttgacacactagtcattacagcctggtggcaacagttctgtttccctctagaagggaggggagggtgtttgtggaagtgacaagaagtcttgggaaatgttacaaggtagggggtatttgtgagtgaaatgtgaagcctctagtgtcatttggtaaaatgtaataataatacttgtgtcctccatagaacttttcttggcactctccatctcttgaccatttcttccatgtgaaaatgatagactaatggacatagaagatgcagggctgtgcacattgttagagctgtagctggtgcatttgatgtttagccattgcattatactaagactccttaagtaatttagcatcttacagccacatcagtaggatctactgtacctgttgcatagttaatatacctagacaagtgttttggataagtttatacacggacctaaagcagaatccttatgtgtatcctacctttattctttcgattatttaaaaaacaagaaagaacttatgaacagatactgttgaccaagctgtgttttactaatggttgcttttgcattttaagctacaatagtattagaaacctacatccagataaaagctttcagatatccctactccaggaatgcttctttacagatgacatcaaggcccagaaaagtgaactgtagtgatcagaattcgtgacggtgtcaatcagttcatgacagcatttacagtaaaaacaagtcccttggtgtgttgttaaatttctttgtatattatcaagcttacagattctgaactgcaaaaggttccatatttattgctgatggttttgaaaggcttctcagttttatgcttttctttacaaatcgtagtgtttaataaccctccaggttaaaatcttggagtgtttaacctccagtctaagtttaaaatctccagagagagattgctagttttgtaactataatgctgtgcatttccttaaaatttttacaatttaattttctcaaagcttttaaatgtaaagaaaaataaaatcatggcatttataggaagagaatggaactagaagtcattactttaagtaaaacaaatctgatcagaaaaacgtgttttctctcatgggaagaacctagcttggaggtatgcataccctttgggggagggagatctgaagggaagatggtgaaaggaagagtggaacctgctccacacagaaatgcagaagaggaactgtttagggagggaggtgactcagaaggaagtacaacggtacctctatgaggaaatgtcatagtgaaatgcgctagtaacaaagaaaaataataaaacaaaccttaaaaggcaccgggccttgggctggtagagtggctgtggaatgatggatgtgttttagcttgctgagtataggaaagctgctttagtacagctgaggaagtcttcctagaagaatcttagagaattgatgactcagtgtggcagtgtagacacacagtggacttgcagacttgtagactagctaggagtccctgtggctacgttcgtgctgcttgttgtgtttgctgtttctggatttatactttgtgattgtaaatgtgaacttctggagattaatacttaaatctgctttgtagttaatcatattagaattgtctgtattattttgtttatatttgtaattttaactatggagggaccataagaagctgaagaaatttgtgttctggtacagaatcctcatcttctttcttttatgttaaaggaggaggcaattggcctgttgtgaaaaaattgtagactttgcctaaaatgtgtgtttttttttttttggcttttacttatagtactttctttctcagagtcctgtagttaggtcttgatatgatgcaggttggatttgcaagctgaccataaaatgtagcattgtgttcttaatgctggaacaatacttcaggttttgaatattgcaatgaagttcaacagttgctacttcctataaagaaaaaatatctcaagggcgaagtaactgctcaagggaacaaagacccatacccttattcagtcaaagaagtgtgttgcccactttactgggaattatttacttgtatagttgatattttcctttgtgaaaaggtggcaaattatccaaatccaaagtcactgagggtgaatcccgactgactgcacatgcgcgcgcgcgcacacacacacacacacacacacacacacacacaccccgcgccagcccctcctgctgtaaattaccttctggggttggtttccacgtgtgccttgctgtttgcaaatgcacttagggttcagtgctgcaatacatggtttgcgggatgtaaactatgaggagagtgtaagaaagaccttttcagaatggcaatgagcgcaggttttggagtaactttccaaacttgagggaagacactgccactaaccatacgtagtgtctgttctccctggcctccagcagtggcaaggaaagtaaccttaatgcttagttctcttattaaacttaggggagggattgaaacgtttcagtttgccttttctttgcttacaagttggttattaggtaggtgtgtgtgttttgactacgtgagtgtgtgtgagagagaatgtgtgtgtgtgattagtttactacctcagtttgtcaactttactaaaataacaatgccaagcttaattttaagagatttcattattttctgtgagaagtgaacttttataacctactatcaaggttatccccctacccctgttttacttattaaatcccctcatcataaaattttcttgcttagtccttatttttaaatcaaacaagttgtggttgctgttgctaaaacaaaaaactcagtataattttcttgtaatgtcaattatcatgtgttctcttctgatgcatactgtttgggctattatctactatctagcagtccactgattcataaagctgtgcagttttaggctttgaaagatttaagcagttacttaatacaaacccatttgcaaagaccacactgccctgattcatagtctttctttgtcatactcaacacctggcttcctaattttgttccttggattctctctgctgtactaagctctacttaattttttccttttctttttgattcctaaaatagaaaccaacaccaaaaaaaaaaaaaaaaagagagagagagagagagaaaaattgctgttttgaatgtgtgagtttttcagttttaggtaagagaaacccaagtaggagaaggagacagagaagacacaagggagatgtttattgactaatagaacctaaaaggtcaggtctggatctaaatgctcaataagtgtcaggaacctctcctttctacactcttcccttttctttcttggctatgtttattcagaaaggatctttgcaagtaataccaagctatggtcccgtagctttaggcttacactcttgactaactgcagtggtaaaagaacacccatttgactagaggttcctagaataggttctgatttccctggcttaaagggtgtgtctttttatttttttatttttttgtaagatttgtttatgtatatgagtatactgtcactgtcttcaaacacaccagaagagggcatcagaccccattacagatggttataagacaccatgtggttgctgggaattgagctcttaaccactgagccatttctccagcccacatctacctcttttttgaaacaaatacttttgctagtgagtggaatcctcttacaaattcatatatctgaattgaagaataggcccaacaccccaaaccacaaaactgtcattatagcttctaaagaaaatccaggtgaggaaagagtgttggccagaaatgatctgtgtcacttttgtcacagagattctgtaagttaccatagaactcgaagcattatgtattccaaacttcagaggttcttcataaaggggaaaaactgaattaagagaagttatggcttcattcagatcgcatagcattcctgttttcaggtcacctgagtcagattcatttaggtcctgtactgcatttgagcaagataaacatgtatagtcctacttgtttcttattaaagtagaggcagcagggctggcctcagtgtgcgtgggacccagcagttggcaggtagagttaggaggatcaagagtcaaagccaccatcagatcctgcagtgtgtttgaggccagctctcccctggggggaaattgaggttgtaatggttggctgtggtgtcacagaggcctataatctccactgttggagcctgaagcaatagaatacctattttgagaccattctggggtacataatgagaccctgtcttaaaacataagatatgtgataatatataccttgtaattttatgggaataatagaaatatgttttgaatattagaaaatatctacaataactactattgtcatagtattagtaaccacattcttttgtttattgaattactgccttgtttattgtggtcattcagaatgtaggtttttcctcttcaagcaaatactttttaggttgaaacttttatttatgagtttagacttggaaaatattttccagtttattaagaaagtttgctcaaatagaaaattacttctttaaggactctaagttgtgaatttaccttctagaagatcatctagaatctgaatgcatagaggatgaacatacattatatatacatatatatatacacacacacactgtgttgttccgtgctaatcccgtatagtagtcttatgacagtagtatagatctcaaagatgtatgtcctccatataagtctgtttttcaaagagccagccaggcatggtggaacacaccttaaatcccgagcacttgggaagtatgagtttcaagacacaaagaaactcttatcttgaaaaccaaaaagagaaaaagtaattagcatcaagtagccaaggcttataaaagaagaattaatgaaaaatacactaggaaaatgtaaaattccaaggcaggcttttcttatcggaatagcattataataaaaaccataaaactgatcaaagatgtcattttacactcacacaggagacacttcatacggacactgagctgttacgaacgtctgtgccaaaaggagtcaactttgttaagcaaatgctagagaaggaaaataaaaagctagactcggaggaggcgcaaagtaaatatgtgcacactgaagacagtgatagagtcggcacctgagtgctttgcagtgcccacttgcttaggttaataactcaccagattaactacaaatcccacaagcttggaaagtagagccctgttacctgtcttgacagttgagtggaactatagtttgaagtttaattcaggtcattatggaaatttaaagccattttcttcaataatttttgagccatttgaagaaagatacattgattattcaccagtagaaaatgaaacaaaaatataaaggagtgaactgaggaaatgctgaactatttgggctagcaaatcgaacattgactacaagaaagagaactggtgttttttttaatagtgtttagggttaaatctgggtttttgtaactgtctcaaagatgaataatgtagcacatgtttgtgtttagctaggcagttaagtcctaggggtgtacgatgctctggcctcgccttgctgcctaggccttagtatggtgcaagcttccctctgtatcaccactgcctgtgagctctgtgagctctattcacacaactgctgttgatctgagaattgtttacttacatatcaacccagcttttttcaaaaggatgagcatataaatgcttgtgataataggtgtccttcccatcacttgagaatctgtttattgctgtacacaagtgtgaaatgtgtaccaaaagctagagagcaggagaaatgccatctgaacattatcagctactatatttttttgtttattttaacttcttttatataactgaatttgctggtttatcctcatgtaaattctattaaggaggtttgtattattgtattaatttttttaaagaagcaaattggaagtttatattttctcagaattcaaatataagctttgactatttagtatgtgatttttcactctagccaaagtcttaaattgccaatcatgatttcaagtggtttatatccatttttgtttgtttgtttttgtttttgagacggggtctctctttgtagccctgttggcttggaatgcacagagatccaccagcttctttcttcctcctgaattctggaattaaagttgtatactgccacttatccattttgatacacatatggagcagagaaacagagaaagacagagagaggggaagagggtggggtcagagttcatatacctgtatttgctagacatcgtattccttctatacttcttagcttgtatgctgtcttcttccccagcagaagggtgtctgtgtctagagtccttatcacagtagaagttgagaggccatcagtaaactaaagagcattgtaatacctttttcttattaacataaaatacctattttctgcaattctgtttgttgagactgggcctcacataggtcaagctggccttaaacttacgattttggttgtacccaagtaccgagattacaagagggtactgccatgccaaaactgctgtttactttaaactgttagattaaatgcttattatttgccttatattgacttttttactgttatattgtgaatagaaaaatattaactcattagtgaatgaattgttacacatatggtatgtatgtctttgtgtgtgtgtgtctatatgtatatatgtatgtgtctgtatgttccttggatgtccatcctctggtgccatctatcttgttcagacagggtctctaattggtctggaattcaccaagtaggctaggccagcctgtgaatcccaagaatctgcctgtctctgctcccccatgccgaagttttaggcatatatatggttacgtggtaccttaaaaaatgtatataggcatgggctaggttggctgaagtgatagctggtttaaagcactgttgttcttgcagaggacccaggttaactcccagtacccacacaatggcttacaaacatctgtaactccagtttcatggcatgccttctggtttctgtgggcaccaggtatacatatggcaaaacactcacaaaatatttcaagaactaagaaaaaaacatatgcatgggttttgggtcatacatgcgattgattgattgattgactggtttccggctggcctggaactatataaaccctgggctggttcggaacagtcctctgcttcatcccctttagtggtaggattacaggtgtgggacaccattcctggttagggtccccttttaaatgtcatatgaattatgtcttttaaatatttgtaatattttctaagccattttatgcttccctaatgtctgcaaggagtggatatagcacaaggataccgataccccttactgcctctgttaacaagttgtattgaatatgtttatcattcagctgcatactctctcttttccttttttttttggactctaaggtcagaatctcatttttcctgcacagaggcaacacgtttctctagtgtactgattctgatatgattttgcttagaaagtatgtaaacaggtttgtgaagacttgcctagtaaacccatctttgagaggagacttgggtatgtgtagctgcagtacagagaccattgactgtctgtttgaatacacagtggcctttgcttattgggttcatagatcagagcattatttaatatttgaataaatgaggtatttaataactacattttaagaatattcttttgttattgctcttatttattctaatactgagttagatttgattgttagccacttaaaggagaaactttgggataacccagttatagcaattttgtataataactttaatgaaactttccttttgaatttgacttctctttttcttttgtctccctcctccctctgccccctgggcctatcagggagtcacaattcccagtcagaggcgctatgtatattattatagctacctgctaaaaaatcacctggattacagacccgtggcactgctgtttcacaagatgatgtttgaaactattccaatgttcagtggcggaacttgcagtaagtgctctaaattcttagctgtctgtgtgtcggaaaactttttaaaaccatatctaaatgtatatgtaaatgtttagaaatacatttaccaggttaaaaggagtatatttctgaagttggaattaattagttcagttgttcaactcactgggttaagaaagctaacccaatgaattttggtgttctttttttgtttgtttgtttttatgtttttcgagacagggtttctctgtatcgccctggctgtcctggaactcactttgtagaccaggctgtccctgaactcagaaatctgcctgcctctgcctcccaagtgctgggattaaaggtgtgagccaccacacctggcaattttggtgttcttcaagtcaattatatttatgggagaaatattctatgttataaccaccagatgcagtctctgttctgacaaaataaccgagagactcttgacagatcagtgtgagaatctgagagacactcttgacagatcagtgtgagaatggcctctggctacaaccaggtcctcatcagagggaaacatgttcactgtcagagacagaaagagaggagcccttcaaatatagagaataaaagggtgtgtgaattgggatgtgtggagaaaacctaagtgtagctttgaagtcaagctctcagcctgtggcttggacttgtcatctcctcactgagtttgcttgttagtaaaataaagaaatcttggtaagtatgacgtcatgacatcagtgtgtgcttgtagtaatttctcttaccttagtgacacctgggagtggtcagtcatgttttacacttaagatattttatttgtattttcaatataacgtcatagtatcagatgtcacttattagaatcccttgatattattttatggatgtttgcagtttgtcagtgattttagtttttcgcctccatgaaatacttcattaaattaacttgttctttctaaaataatttttatgtagagaatcagtttcttcgtcagataggtgtgattgcccatgagtatggcaactttgttaagggcccctaatttatttatatagcttgttggaaagttctaaggaacttccatactgattaagtatattagtataattttaaaagtgtgagtacaaggcacaatggactattgaaagcaattgacctaggaacttcactaggttcattgagaaggaaaggggataggaactcatgttcttcctatagtttaggcatcaatatagagctgtaaaggcggtatggaggcaatacggaaaagctctgctcaggaatctcactaggtactgactttttgtcagctctactttgcttattgattaaactgctgttgggcttttagtcggacaacattatgtttgcatctctacgcttaaaaaatattgactactaaattctttttaatctttaaaaatttctagttacttatttatgtgctgggatttaacactatatgttgaagttatattaaaataaagctcatgagattcatatgtataaattataactgccatgttcacaaaaggttttggaaacaatttcatgaagaaagtcggtcactggagaacacatgcagtttggattggcattgagtgtgaaggaaatattacaggcagtagaaataaggtgaccgaaacacagcagaagaactggacgagaaatagaagatgtgattggctgtcacaggatcttgaatgggatgtaatagacttggactattggtgtcacgataattctaggaacttcagctgtgttttaaggacaggcccatgttgtatagacagttgtgatgtaggaggggtagtacagccagctctgtgacttggcagatgttgctagagcgatggaatgtactttgtttggattttctttagttgttcatacccagagtgcatttgagatttggagatataaatattatcactttgcttttaagttattttaaatctgatgagatcaaggtacttgtgaggatctgcagtggggctgcagctctgtcttggtggtgtagtgtttttctcaagctagagtgcaggattgccttcagtctccagcccgaggaggaggaggcatgagtgaaacagacagaactagtgataaaaggtttagtgtcttctgcagagggaccaggaagtgtttgccaagcgtttgtcttattaccttactttttctacgtgaagagaagttgctgtataataaaaatgatcagtttgcttttgtgtcaggatgcactctcatccttgtttttgtcaagtccagatcctgtcatttcttcagaacatagttaattaacatagcatatatttcagatgtgcatctcagaagtcaagaggactgtactaatattagaatattgatgttagaacttccatccctcctgctgggcgtggtggcacacacctttaatcccagcacttgggagacagaggcaggcggatttctgagttcgaggccagtctggtctacaaagtgagttccaggacagccagagctatacagagaaatcctgtctcgaaagaccaccaaaaacaaaaacaaaaaagaaacaaacacacaaacaaaaaaaacaatttccaactcaatgggaaactcatggtctataggaggtcgttttgtctcagtggtcttagaaaaagctgctgttttttcattactgtacactacagctggggacattgtcaaggaaagatgccatgacaaggcaattcttataaaagagttaattgggacttgcttacactttcagaggatttgtccattatcaacctatcaggaagcatggtggcacacaggcagatacgctgagagttctacatccagaggctggcagcctctacctcctgagtgccagcacttttaaaagcacgggctaccatcacccagctatacatgttttttatttttttaaacatattatttttattaattccttgagaatttcagataagcattcagtgtatcttgatgtaactcatttcctacttctcccaacagccttgtctagttccaggacagccagggctaactaaacaaagaaactctgtcttaaaatgccaaaaccaacaaacaaaaacttgtgaactggaggtcaaaagaaagatgatcagtattgtatatttctatattataaaggcttaggtttacatcgtttattaaaatattcttgtaaagttgcctctatgcagttcacaaaagtcccaggaaattgttaaagttattttatgcttaaagtagtttgtgatgtgtcttttgcatgttaatgtaaagctttaattgttaatatgctgctagagtctagtcttagaacttactgtttgtgaagtagcaattgattatcatatctgtaatagaactttacttaaggattcagattgaagaagtccttacatgggttggttatgtttttacatattttatttctactgatttgttttagataagcagtcaatatgaagagttatcattttgtgtatatttctgtttattttatcattaaagtagtttttgataagttgaagacatttcttgtgaaatgatcctatatgtatttaaccacacagatcctcagtttgtggtctgccagctaaaggtgaagatatattcctccaattcaggacccacgcggcgggaggacaagttcatgtactttgagttccctcagccattgcctgtgtgtggtgatatcaaagtagagttcttccacaaacagaacaagatgctcaaaaaggtttgcaattcagttctattgtgtagaaatagccattcctcaatgagtaaccacagaccttttgcttaaagcctttggttaggaaattattctgtaaggagagactatttctctgccatctaccaaactgattactcttttgttagattggcagactcttcatttctgtggcattgatgagacatgtaagcagactcacctgccccgacccttagtttgttggcttttttggtttgttctatgttcttatgttgcacaagccatgttttacccatttgggtttatgtttcttgagagtagactattttatattttgtttccccaggttactgagtatttagtcagtactgagtatttagtcagtactgagtatttagtcagtattacttagttgaaagaagtggttgtgaagaagtaatagtaattaaaaaaaaaaaaaaaaacataccaaaaaacaaaagggtggaaacagctaagcatatcctctgtccagggttgctggggcttgagatacacttgggaggaggagagagctcaggtgcagtgtccttggtgtgaacatttatccttggtggctttagtacccttagttgaatgtgaacattaaaatgagtcttaaggtgtagaagttttagctggatcagaagaagataccagttttgtgcagaatcaattcccgtgaagcacatagagcagtacatagtccggaagggggttttataaaaagcagtaaaaggagagcttacttactataaaaccttgcaatgtcagatcaatttatcaggagagataatttcttttttctaggatctacatagctaaagaagaaaaaaaatcacaaagttaaccaaattaatgtttttatcatggacctctcatgaaacaaaaaagctaaatataaatatctttgagttactggtttttttgtttattttctgagaccacaataaatgtgagcaaacatttcaaaaattaggtatttggaaggctttagtttttttccctcttacttcatttgtttgtttgtttgtttgtttgtttgagagacatggtctctctgtctagctctagctgtctcgaacttgttttgtagacaaggctagtctcaaactcagagatcgattgcctctgcatcctaaatgctgggattaaaggcctgtgccatccctgcccgctatctcttacatcttgtaagactctgattcacataagagtgtgtgtgtgctcacacttgtgtgtgtgcgtgcgctcacacatgcatatgtttttctttccttgtttgtttgaagacagattcttactctgtagctcaagctggtctggaactcactaagtagctcaggttggcttcatataaaacagtctttttgctttaaattcccaagacaggagccgcctctcttggctttatattgcttgtaaaaagagataattacctcactgtctccttgtagttttagagatgtgctgtttcttactccagatctctgaagattggatatttataatatatttgtatattttagattactttccataaatttccatttatcataggttttccttactaattatatgcctgttctgatttaaataaaaacagttttagaaaatcagtgcacatcagtgagctacatttgcaagtttaaataatattaccgtgttttcttctaagtaaaatatgattgccaactgaacaactttgttaaaataattgagatcaataaggtttgaaaagggctggaaacacgactcagcagttaggagggagcgctggatgttctggaggacacaggcttggtcctcagcacccatctgttgtcttacagttgtctgtaataccagacctgggggggaatctgagtcctacctcgggcctccatggacactggacagatacaggggccagaaatgtagttgccatagggatcaacttactagggcttctgttgaagtagcaacacctgatctttgtttatatttacatatgcataaactcagaatatttttaaaatccagtattagaataagaagtagttttcttaggattttaagataaaagtaagataccagattttacatgattatattttttatttgaaattttagaaaagataacttagtgaataagaatattccacaaggtgtttgccttcacttaagaatatcaaataatatatggtttatatgttgactatttgtggtacatttttaaataagtgaaatacatcttccatttcttttctgtttaggacaaaatgtttcacttttgggtaaatacgttcttcataccaggaccagaggaaacctcagaaaaagtggaaaatggaagtctttgtgatcaggaaatcgatagcatttgcagtatagagcgtgcagataatgacaaggagtatcttgtactcaccctaacaaaaaacgatcttgacaaagcaaacaaagacaaggccaaccgatacttctctccaaattttaaggtcagttaaaaccatttgggggtgggagggtgtgttttattctgattgtgaagctaagagctagacattgttctagtattgttcaatatgtaacattcccgagtggttgcgtagcctttccttttctgtttattactccacttgctctccttatttcttctcctcccccctccccatcttgagtctttcctctttcctttttcctttttaaattcttttttcattccattgtatttgtttcattcacatgtaacaaactcaaccatagattattatattattattataacataataatactcctaatatttaaaatagaaaatgaccttcacacatgttaagtgaacatagacctaatattcagtgcctttggatctcaagtatgttttaagtttcctttagaatcttcaacttttcattttttccttttctctaaatttacatttctatcaaccacctgcttcctgatttttattttagataggatcttggtatataggctcaggctggccttgaactggcatgtctctgcctcagcctcacctggcaaccatttgcttttaaaatgtatttctctctagttttactgttttaatatttttctggcaatgtgcctcaatgcttgtctaacatgagaaatgccttgagttgaacaccaagtcctacaaaacaaccagtaaaaaaataaagcataaaatagttcgtgtagataaaagctgttttcattgtcactgtaaggtaagtcctatgaagcatgcagctggcaaagaaagtctggtcaagtggatggtctgtaaagagggttgtctgcgtgaaatgggtgcatttaattatgcaaattgtatgcgacctacagaacagttatgtgggggaaggaggaagagtagaaatgagaatctgaggcatgaatgagcttggtatattcagtggctgaagtcatttccactgagtcacaggaacccagtcttgatgtagcagtgtggagtgctgaacacagagaaggtataatctgagctttttaaccttccagttcacttcctaaattgagactggctcagaaactagggagaggagaggccgtggggctgtagtggcaaggagcagtggtaaattactaactttgaagggggatttttaaagcaaatgttggttataagaaaaacttataatgacttgcataatgggaaaagagcaaagatatgatttaaacctggtctctgccaattgttcactagagattgtcctttagcatgtggcctagacttcagggtttttgtttcttaccttttaaaatggaaataacctaagaactgcagccatgcatgtaaaggctactgatcaatcctcacagcacagagtgctctgttagcttgcaccattctctcgtcagatgactgttgtagagctagttgaccttattaatcagtattgtgtagtgtcactaatgtaaacatagagttgtctcagagattgcaagagaaatgtttttgaaacataagcacttgtaaattgaattttgtgatttagtgagttcattgccttcagtttgcacttttatagaattatacagtgcttatggctgatgtattttaaaaatagctttcaactcatcattgtgtcttttgagagcacagtagtcaatcttcaggtcatctgaaaagcagtgcccttcagaattcattttgttatagtactttcaagtaaatctgcaaaacagaatgtctttgctaatacagaactcattctaattgttcattttcatcttaaactttctttctctaggtgaaactatactttacaaaaacagtagaggagccatcaaatccagaggctagcagttcaacttctgtgactccagatgttagtgacaatgaacctgatcattatagatattctgacaccactgactctgatccagagaatgaaccttttgatgaagatcagcattcacaaattacaaaagtctgattttttttttcttatcaagagggataaaataccatgaaaaaaaaaaaacttgaataaactgaaatggaccttttttttttttttttttttttaaatggcaataggacattgtgtcagattgcagttataggaacaattctcttctcctgaccaatcttgttttaccctatacatccacagggttttgacacttgttgtccagttaaaaaaaggttgtgtagctgtgtcatgtatatacctttttgtgtcaaaaggacatttaaaattcaattaggataaataaaagatggcactttcccattttattccagttttataaaaagtggagacaggctgatgtgtatacgcaggagtttttcctttattttctgtcaccagctgaagtggctgaagagctctgattcccgggttcacgtcctacccctttgcacttgtggcaacagataagtttgcagttggctaaggaagtttctgcagggttttgttagattctaatgcatgcacttgggttgggaatggagggaatgctcagaaaggaatgtttctacctgggctctggaccatacaccatctccagctccttagatgcacctttctttagcatgctccacttactaatctggacatccgagagattggctgctgtcctgctgtttgtttgtgcattttaaagagcatattggtgctagacaaggcagctagagtgagtatatttgtagtggggtacaggaatgaaccatctacagcatcttaagaatccacaaaggaagggatataaaaaaagtggtcatagatagataaaagacacagcagcaatgacttaaccatacaaatgtggaggctttcaacaaaggatgggctggaaacagaaaatttgacaatgatttattcagtatgctttctcagttgtaatgactgctccatctcctatgtaatcaaggccagtgctaagagtcagatgctattagtccctacatcagtcaacaccttacctttatttttattaattttcaatcatatacctactgtggatgcttcatgtgctggctgccagtttgtttttctccttaaatattttataattcttcacaggaaatttcaacttgagattcaacagtaagcaggttttgttttttttttttcctagagattgatgatgcgcgtcctcagtccagtggctgtcagacgttcagcccctttgaccttacacattctattacaatgagttttgcagttttgcacattttttttaaatgtcattaactgttagggaattttacttgaatactgaatacatataatgtgtatattaaaaaagtcattgtttgtgttaaaaaagaaattagagttgcagtaaatttacagcactgcacgaataataaggcattgaagtttttcagtagaaattgtcctacagatgctttatcgacttgctattggaagaatagatcttcttaaatgtgcagtgttgagtcacttcgttatagtggtagagttgggattagggcttcaattttacttcttaaatatcattctatgtttgatatgcccagactgcatacaatttaaagcaagagtacaactactatcgtaatggtaatgtgaagatgctattacaaaggatctcctcccaacccctcgggaatttggtgtctttcaaattatatcttgaccttgacatttgaatatccagccattattagatttcttaatggtgtgaagtcccattttcaataacttattggtgctgaaattgttcactagctgtggtctgacctagttaatttacaagtacagattgcataggacccactagagaagcatttatagtttgatggtaagtagattaggcagaacgccatctaaaatattcttagaaaataatgttgatgtattttccatacctcatcagtttcactcaaccaataaagtttttaaaattgtaacaaagctcttaggatttacacatttatatttaaacattgatacatgaatattgactgactgttgataaagtcagagacaacttttcctgagatctcaccatggaaatctgtacacccccttgtctttcctaaaagctgaaagtggctgactaaaatgcaaagcagctgttgatgttttgaagatagtgataaacactgttctttgttagttttgggcacagcatgctaaactataacttgtattgttccaatatgtaacacagagggccaggtcatgaataatgacattacaatgggctgttgcactgttaatatttttcctttggaatgtgaaggtctgaatgagggttttgattttgaatgtttcagtgtttttgagaagccttgcttacattttatggtgtagtcattggaaatggaaaaatggcattatatatatattatatatatataaatatatatattatacatactctccttactttatttcagttaccatccccatagaatttgacaagaattgctatgactgaaagggttttgagtcctaattcaaactttctttatgacagtattcacgattagcctgaagtgcattctgtaggtgatctctcccgtgtttctggaatgctttcttagactcttggatgtgcagcagcttatgtgtctgaaatgacttgaaggcatcacctttaagaaggcttacagttgggccccgtacatcccaagtcctctgtaattcctcttggacatttttgccataattgtaaaagggtagttgaattaaatagcgtcaccattctttgctgtggcacaggttataaacttaagtggagtttaccggcagcatcaaatgtttcagctttaaaaataaaagtaggttacaagttacatgtttagttttagaaaatttgtgcaatatgttcataacgatggctgtggttgccacaaagtgcctcgtttacctttaaatactgttaatgtgtcgtgcatgcagacggaaggggtggatctgtgcactaaacggggggcttttactctagtattcggcagagttgccttctacctgccagctcaaaagttcgatctgttttcatatagaatatatatactaaaaccatccagtctgtaaaacagccttaccccgattcagcctcttcagatactcttgtgctgtgcagcagtggctctgtgtgtaaatgctatgcactgaggatacacaaatatgacgtgtacaggataatgcctcataccaatcagatgtccatttgttactgtgtttgttaacaaccctttatctcttagtgttataaactccacttaaaactgattaaagtctcattcttgtcattgtgtgggtgttttattaaatgagagtatttataattcaaattgcttaaatccattaaaatgttcagtaatgggcagccacatatgattacaaagttcctgtgcatttttctatttttccccctccttgctatccttccaagcaaagcatctttctgtcatcttggtagacacatacctgtctactcatggttaagaagagcactttaagccttagtcatcacttaataagttattccaggcacagtaaaaagttcaaggttcttggaaaacggtgcttatttctcttcttataagccagatgtctgaagatagccctaaccccaagaacgggcttgatgtctcaggtctgttctgtggctttctgttttttttaacactgcagttggccatcagcacatgggaggtttcatcgggacttgtccagagtagtaggctcaaatatactatctcctttctaatattcttaaaggctaaggagtcctttcaatataacagtaagataacttgtgatgttttagaagtaagcagaccattaatgtcaatgtggagtcttaatgttacatgaagttgatagtttctctgtgacccatttaaaaatacaaaccgagtagcatgcaattatgtaaagaaatatgaagattatatgtagtcacacattttctttagaattcttagtttggtgaaaacttgaatataaaggtattttgatttatatgacattttgatgatatttgaaaaaaaggaatttcctgacattttgcttttagatcatgtcccccattgtgctgtaatttaagccaacttggttcagtgaatgccatcaccatttccattgagaatttaaaactcaccagtgtttaacatgcaggcttctgagggctcccggagaatcagaccttaagcccagttgatttacttctaacgtgaaacttcgagttcctgtatactttgctagataatttgtggtacatctaaagcttagtcttaagtggcttgtgtgtggattttattcaacattcttgttgctagggtagagagaaatgttgctgagtagaaacaagagtacccagttcaatgtggtacagagagcagtccctaaaatctgtacacagtgtaatggaccactttaggagtcaagaggctgatttttcctatgaaattacattgcaacaggaagccttctagtatagttccttttactgttagaatatgtttttatgcatacgctatagctgctttcccatcttccaacaacaggtatcaggatgtaagcaagctttaaacagtgtgaagatggcaggatagtgtcatcggtaacagtcctctgactctaaatgtagttgctctgtaacactttgtgaatataacatcacaattctcatgtccttggggggggggggcatacccagtattagtatgttttagtgactaagcaatcatttttctgtttactcatgtacattttctctttaaaactaaaacctgtactgtgtatgtctccaaagccttttagcttagtttttaggaaatgaacactgaatggatcactttttagtgtagcaggtatgggatatgtgcattatagagagaccttgtcagctctctgggcctatttgaatgtttattgttggtgtgaggatggtaggggaatcagtaaatacaagttacgttggtttagcagagcaagctcagtgtgggtatttctctttgaagcgtggtgcgtgacgcactgtgagtagagaatttggtcaccctttgagtcctcttgcattttgcaaacttgctcagcaaatgcgtacctaccttgccccctaggtaaaagcaggaactactactgatttatctgtcactcagctgtctttatatgtgtgcttctgtgacttgtatcacacaagaatcttaaagatttcacaaattgttaccttttagctctgaatgttgagtattctggtgggctaacaacaagacaaactcttgacagtcatttgagaattttcatgaaacatttagctgaaaacattttataatttatgaaaaaaatgtgttaccttaaacttttacatatgtgggagacattaactgccatatttgagcatactgaattttaaatttaaaataaagctgcatatttttaaatgaaatgtttaacaaggattcatattttttgttttttaagattaaaaataatttatgtcttctcatgtggaacctcatctgtcacaatggttagattatacagaatggagcaaggcttgtagtggtttagcttacagtaaaattcttaatgtttagatgtgtttacttactggctgttatgtatacttttgagattttccacctgttctgtgtagttttctaaatgatactcctacttaaaaacagcattttagtatctattttctgtctccattaaatggtcctcattttctattgagtttggaagtgtgcacattgtgtgtgtgtgtgtgtgtgtgtgtgtgcacacgtgtgcgcgcccgtgcgtgtgtctatttgtggagtttgtatgggagaattagttttgaaagtgctagaatagagatgaaatttggttcaagtaaaattttcccactgggattttacagtttattgtaataaaatgttaattttggatgaccttgaatattaatgaatttgttagcctcttgatgtgtgcattaatgagatatatcaaagttgtatattaaaccaaagttggagttgtggaagtgtttttatgaagttccgtttggctaccaatggacataagactagaaataccttcctgtggagaatatttttcctttaaacaattaaaaaggttcattatttttgatgatttgcttcttagctttttattcatccgagaatcaatccctgctatgtgtaacaggtatttggactatattacttagtgaaagaggaacatcctgatcttactgttgaaaagtttatacttcaccagagtaaactacgtaaaagctgtggggtgtgctaggaagtatgagacctcatggctcccttgcatgtccatcctcagagtttagattctgtttactagagcattactagagtgaaatagccatgaaagtgcagagaaacttccaagtaaaggcagtgagagatccctgtaccctcctgtctttttccttccctagtgcagtgtcctgaagtgtgtcctagtgtgtgtcatggctctgccagcccaggtcttgaggtaacacctgcagtggagctcctgagggtcttcctctaccccctttcaacacaagcaagaaacaataatattttgggttgctattctaaccaatatgctttgaaaaacttagctttcttgagcagcaaagggaattaataataaaacccaacatatttagattagtttgtccaattaacaggatgcaagatgtccaaatgtttttttttttgatgagtagttattgcggtacgtcaggcattgtgaacgctcggcatgcacttgggtgaacagcagtttcctgtctgtgaagtttgcattctagcaagaaaaatagagcaaagataaatgtgtcagctcacctcaagcagagtgacttcagtgttggataggttatttgagagtggcttcagcaggtgtggggggtttcagtagactggtgcaatttgtacccagaaacttccacaaaactttactcagacttggagtttgaaaatgtttagtatatctaaggagaatttatagtaacctcatcttaagtagccatgaccattctgttgtctaaattggtttggtaaagcctgtttaaacaattggttcctctttattctcagagttaactttttagtgttctctgtgtatgtttcaaaaacactaagtcatttttcagaagttaaaggctctattttgaccttaaatagttttgtaatgttgaagagtcatgccatttatgatttgttgttggacagtgagttataaatgttgagttactcaggatatgggtgtgatttatgacctggtgctctatgtattagttcctaacccataaccaaacatcctgaaaaataccattatctaagtatcagaagcagcatttccacacaaagataaaaatccccacctctcctgcttcatctcaaagattgaagtcaaagtgtttaataatctgctacaatggacacttgaaacattttattcattacatcattaacattttagtagtggcagggtccactcctcagatcaggcccaggactaaggggtgaggtcttttatttatttgtttatttctgtgtatacacgttcacatgtgtgggagcatgtatggcacgtgtatgtatatgtgggtggagtcagagcttaatgttagttgccttcctccatggctattgaggaagccatctcttcctaaaatcttcctaaatctcttcctaaacttggtgcttggtgattcatctgcaattacagttggatgtacagcccagcatgtagtgttgggatcataacactagtcctcatagttgcaaagcaagtactttgctcagtgagccatctccccagtccctagggaggaaatcttgaaagaaaatgttgactttttttttttaattcttgattttaaaataggtttaaaagaatttgatagtcatctatagatagaccatgaatctgagcaaggagaggtatataggaagatttggaagggaggaaggcaaggggagaaattatgtaattaaattacattatcgggctggtgagatggctcagaggttaagagcaccgactgctcttcggaaggtcctgagttcaaatcccagcaaccacatggtggctcacaaccatccgtaatgagatatgatgccctcttctggtgtgtctgaagacagctacagtacttacatataacaataaataaattaaaaaattacattgtctcaaaaaataagaaaataactaaaaattttaattttacttaatgttttcctgtgaatattattgataagaaacctttttgtgtggctgcctttaacagaggagccacaagctatattcttgttacttattacgtttgtgtgtttttgtatatgcaaatgtttgccatgatactccatgtaggttaagaggacaacttgtcaggagtcttttttctccttccactatgtaggtcccagaagttaaacttaggttgtcaggcttggtgctcatctcctgagccttctcatctgcccataccaggtattctttagcaagtacttcagtaagtacagccattgtaagtcacaagagcaattagacttcttcatggtagaaataattagctgatggaaagattatattttaggattataaatgttcttggggggggtttgtttttgtttgggagtttttggtttgttttgagacagggtacttaggctggcattatactcacgatatagttctggctgacctgcagcacatgatcctttggcttcagtctcccaggtaagaatactggaattacagatgtgttgccaacacctctggttattagtgatgtgtgtgtgtgtgtgtgtgtgtgtgtatacacacacagtgaatgttttactcttttggtaaaaatggaggtctgaggtaaaagtttcctatgtcagctgtccatagggaagacttaaaagactttgcttcaaaagattgagggctggtgagatggctcagca

SEQ ID NO: 4 - DNA, 9001 bp, Mus musculus
  source 1..9001: mol_type = genomic DNA
ggcagatttctaagtttgaggccagcctggtctacagagtgagttccaggacagtcagggctacacagagaaaccctgtctcgaaaaaaaaaaaaaaatccctttctctggctataaggatggctcagtggttaagagcactgactgctctgccagaggtcctgagttcaagtcccagcaaccatctgtaatgggaaataatgcactcttctagtgtgtgtctgaagacagctgtagtgtactcatataaataaaaaataaatcctaaaaaaaaaaagaattcccttcctcttattgataccctttcttgtctctagggaccccccatatatctttctagacatttctgagaactcatgtaaatacatgctgagccccctctttgtagtttgtaacctttgctcattccataccattttaacaaatattttccttgaaacactatttctcacccattgcatggaggtatcacataggactttatcaggcatcctgttctccagtgtgtggcttagagccagtggaatgcacggcgtgtccgagaaccacttcacacagggaagagaatacagatttttactcagcaagtaacaccagctgggaatggtggggcagacaagcaatcctagttactagggatgctgaggcaggaggatcttaaattcaagtttagtccctatctcaaaaattaaaaaagaacccctctaaccccagcaactgagaggcagaggccagggagatctctgtgagttcaaggtcagcctgtctgttctacacaatgagttccatgagagccaaaggtacacagtgtgatatttttaaaaaggtatgtgtgtctttttttttttcaaatttttattattttcttcatttacatttcaaatgctatctggggagtcccctataccacgcccccctgctcccctccccacccactcccacttcttggccctggcattccactgtactgaggcatataaatttgcaagaccaagggcctctcctcccagtgattgtcgagttggccatcttctgctacatatgcagctagaaacacgagctccggaggtactgggtagttcatattgttgttccacctatagggttgtagaccccttcagctccttgggtactttctctagctcctccattaggggccctgtgttccatccaatagatgactgtgaacatccacttctgtatttgccaggcactggcaaagccttacacgagacagctatatcagggtcctttcagcaaaatcttgctggcatacgcaatagtgtctgcatttggtggctgattatggcacggatctccaggtggggcagtctctggatggtccatccttttgtctcagctccaaactgtgtctctgtaactccgtccatgggtattttgttcgttattttaaggaggaatgaagtatccacacgttggtcttccttcctcttgattttcttgtgttttgcgtgtgtgtgtgtgtgtgtgtgtgtgtgtgtgtgtcttaactggcagagcacttgtctgtcatgcagggggcggggtggggggtggggactgtctaatctccagctctagtaacaaaaataaaagaagtaaaaaataagtaagaaacgggggtgtgtctagagatagaacatgggctttacacattttagacatcatgagaaaataaagctggaaatgacactgggcatccatcttggcgcatctcaactttcacactgcaaccgaggcgcgctgtgcaaagtcagtgacaatccgcatttccagacacagtgggttcagaccttccaggcgcgcacgcgggcctcgtgttctcggtttccgcggcgactcggccgacgtcacagttagaagacaatagcgactttccccgctcaggctcctcgggaactttctcagtccgcacgctccaggagccggagctaccctccgccccgcccccagcgtgccccgcggccagggagctccacgaagggcgggcggaggccgcgggtagcgattggttccgtgccaaggtgggcgtggtcagactcaggcctataaaagctccgtggcgccagggcctcgtttttttgcgcggtcctttcctgcggcgccttccgtccgtcggcttctcgtcttgctctctctggtccctccggaggaggccgccgcgcgtctcccggggaagcatggcgatgaaagcggtgtgcgtgctgaagggcgacggtccggtgcagggaaccatccacttcgagcagaaggcaaggcccggggcgcggggcgcaggccgcggtgacgcggcgcacctgtgcgggagcacgccacgcccccgccacggcctgagcccgctaagtgctgagtcaccgtggcctggggcaggggctgggcgccgggaagcgaggcccggggcgccgcggggccttccgggcgggcgggggcctccccgcgccccggagcggctgggcctgcccgggagagccggcttggcatccgttatccttctggggctgctgcttttccggtgtccgtgtcccacaggctcagagccccgtggccaccggctgcgattgttgtaagaatatttgaacccggtggtgccagaccggactaaggccgcaggacgcgtttgcggcactttaaagcaaagtcctgggctgttctgtactaggtcagggtcgtgtcgcaaggcggaaagaaagagatggccttggacagccgtcccttgctttgcactccagagagagacccggctgtgggttttttctaccacagcgagtttctgagcacattttggaaaagtacatagagatattttcgaaaatactgtgaccctgcaaaaacacatgcgtcacagggaagatgtgtgtggtaaggttgtgtccagagccttagggaggttaccgttgttgtattcaccttaatcccgagagaatatttgataaatgagcgttatgtgctctctgaagtggtggacatacgtgtgagaaggcagacaccatagtgaatcccaagtgtttggtttacgacgagaactgataacggcaatttagagtttttcgtaactagcctcgtttccagcagtttcttggcattgaaatgcgttttgttgttttcctgtggaagttttttgtttttgtctttttctcctccccacgtaattcactgtgagaaagacgaagttcggctgggtcttacccctgtgtgtgggtttctgtcatcttccaccatgccatgccagagcagctcgcactatttttgtgacgctgcaaactacacatcgctggtgccctttgtacccaatgaaacgatagttaagcattccagattggcagttgtaatcaaagctggttgatttaacctgttgccaacttttcagaatcagatttttctacccaaagttcatattcccttattctgttgcaaaagttggacatttaaaaaaaaaaaaacctaaaaaatgattgtccttgcttgttggtcggttgctcttacattttctccctattgctacactttctggagcagtactaatttgaattttgggtgttcttttcttttttgttaagtggcaaattttctagatttggatagctaatgagattttttttttaaggtagctctggttagacccaaatggatctccacaggcagtaggacaaaggcattttctgaccactaaataaaaataggggaactgataaaatcactgaatgtggagaacagggttctcggcagccagtgttctgtaagagtcaagtctgacagtgcagtagccatctcttccccaggcctggcattcagtagcccctgtttgttccacctggtgctttctaaatgctgttcagtccaggtgcctgcacacatggcatctggcagcaagtgttaggagaagtgtgacagggagagagaggcctagagctgagcgtctccagagccaccctgtaggaagtgggtctacttggatctgaacataggtttgattttcactgttgtgtgttttgacttgagctttttactgtgcttggttagggtgtaacccagcaacagccctggtgcaggagtatttacactcaaacttgatgtcttcatttttgtatttttttaaatcaaggcaagcggtgaaccagttgtgttgtcaggacaaattacaggattaactgaaggccagcatgggttccacgtccatcagtatggggacaatacacaaggtaggtcctaggctggctagtgaccagtgatggaaaggaactgagtcaggacccaattactaaccatttaaaactatctcgtttgttttctttttcttttagataaagttaaaatgaccacttaggtcaaccttggaaagtagccacaaaagtattttatttagtatcaagtattgcttgcttccttaagtgtgggaaggtaaagaaggtgatttttcttcattgtaattataattaagcagcaccttgcttattctgggtgtttattgggtgcttatttgggtgtttggagctgggcgttgaggatggatgcattaggcagagtgtctaaggacaaccatgccttagcatgagaggcatagcgggacagaagtgacaaaaactgaagattcaatataaatgcttaagtaagatttattttctctatttgggattagaatcaagtcagtaaaaagtagtggcttaaattgcagttagtgaacttttaccatattggagtaatgatctgaatttgcttaccgtcatttaagagcctcatccatgttgcgagagccttttcctttcctctccttctcccctgccttcttcccttctccacatagcccacggtggcctggaatttagtcttgtgtgtgctgtgttaaaggcatgtaccatcaacctgtgtgctatgtgccataatttgttctacagttacttaggattgggtttgacccatttgataattactaaagttaccccgagttgcctctggcctggtagctttgatttgttaagctccttccagaatcctgcccagttcctattttcttggtctgagtaaacactggaagtcctgcatataaaaggacttgctgcattgttgagctgtgccttgtgactggcatcccttagcccacatgagtagtgtggtacacctcctggagttgaggacaccagccctggcccttgggaacaagccatctaacagtctgcctgccccaagtaaaagctagacaggtgagctgtttggtggcacatggtctagaaagataagtatttttatcatgaagtatgctcccttcttaaaagccaaggtctttaaatgtgggactttaactttagaagtgccattaaagatcacatctgttccagttacaaggaaggaacaagagccaggcatgctgtcctgacactgccatggccctaaggctaaggtgggagggtcataggtcgcagatatcctgagctgtagtagtgagacactgtctcaaaactcaaaagcaaacaaaaagaaaaatgtgacagtctaggaaaaaaaggtagcctgagaatgtaaggctatacagtgcagctacttacaccagggcgctgctgcctgtttttatcgccccagcacataccaggtcagtgtttgctatgttggaggttgtaagaatgcctgtgttgttacatataggggtttacttcataatctgactgctggtttctggtaaataggctgtaccagtgcaggacctcattttaatcctcactctaagaaacatggtggcccggcggatgaagagaggtgagcagcacgctctgtatgcatggtggaggagaggggtctgtggaggaccccagtaagacagaactgcatggcctcctgcctctgcttttgtgtttgtttccattcacccaactcactcccacaaccccacgtgctagaatagcttctgttgggtgaaggagctgacaaatgtggactcttaaagtgatttggttttgtagcatttattgaagatgaactaatacaagtgccaaaaggaaccaatacagaaaatatcatggataacagtactgtcagtcactggcaaagtaaatcattgtataataggacgctaatgcagataatgaaaactagttgagattccatttgtatgtgaaaccttaggaaagtcctaaataaagaagggctagcctgtttttagaatgggggcctgggagcaaacctttgctaactcaggagctggcatactttactaaagccccagattatgactcttctcagagcactacctttaaacttgaagaactgtctgtcaaggtatcctgtagctacctgttttgaactttgtgtttccagacctttgccggtctggaaaagccatcatagttgataatgtatgtgtactttttcatccactcatacgtatttgacttagtcagattttaacttagttattgaactctagtgatgtgaaatagacatcattgttcatccacctgatgctgttttaatgttaggcatgttggagacctgggcaatgtgactgctggaaaggacggtgtggccaatgtgtccattgaagatcgtgtgatctcactctcaggagagcattccatcattggccgtacaatggtggtaagtttttcatataaggatatatacataggatttcttctaacatagttatgtaccttcccatgactttatggtggttaaactagtttctaaagagtcacataaattgttaagagttcagggtaggaaaaaagttcttttattggctgtgatagtaaagaattaatttgcctaggtcagttaagaacactgttgtgctgaaatgcagtagaaagcagttacatttgatgagactggatctgagttgaggatacaatagtctttagtctaaaacagccggattttcttgccatgattgcccccccccttgcaacatttcgttgagtctaaaatctgcgatggatggcagtattcaagtctgtaggttatcgcttggttaccatatgggagccgtcttcccaagttaccctcgggagatgcatctgggtcatgcagaacaccaagtagtaaaggctcttgcccacctcgggcagctaacttttcagtaggcacttccttccttgcagttgaccctttatccttagaatgctcttcagccctattggtgaagcagaacagtcattcataagtgttgtaaaataaagctttagagtcttgttgctaagtagagatacttagaattgcctcttatgtgtaggcctatagttctttcaacatgagattttgatagagaaatttgtaagaatgactactgtgtagttggggaggagctaagatcagcatgtacctggtagttacttgggtcttagtatttcatctagaaatagccactagcaaggaaaaacttagtggtctgctcttaactgctagtatttaagtctgtagtattgctgggaagaagtactagttacttgatcattcaaacctaaatgttcttcttttcaaaggtccatgagaaacaagatgacttgggcaaaggtggaaatgaagaaagtacaaagactggaaatgctgggagccgcttggcctgtggagtgattgggattgcgcagtaaacattccctgtgtggtctgagtctcagactcatctgctaccctcaaaccattaaactgtaatctgaagagttgtaactgtgtgactcctttgactgggctaaggacagcaatgacagctgatggagactgtgtacaactcactgaattcaaatctgtttctgtgcctttccatattttgccagactacacaggtgataagctgaaattctcatttgagcctgttagtaaatatgtgtggcacttattttgagcctattaatgtgtacaaaaaaaaattttaagttagctctatacattgagcatcaataacagactcaatgatgctaactcatagtatttcattttgaaagtgttttatgtgataccatcaaaatggtgggtggtagcccaaacaaaatttgagcagaaaattttctgccccttatcagagaaattattgaaagctctcaagattcagagtacttaaccttatattttaaaattgtattaggattagatgtcatgatttaagaaaaagccctttagtaaacttgtatcaaactcatagaaggcaaacatggagcctcagctagctctactagccaagtgaagttggtaccacccatctttaaggttggcactcagggaaaacacaatagctcggggaatgacaccaagtttgactggaggttctggctaaatcgacttttatagccccaggtaatgaaattgagtgccttaatacccaagaaagagtgcctttgaaaggaaatattaacaggcttgtgactatctgaaatagttcaattgaagtattttcaacaaattgggtgtaaaccatagttctcactgatatactgaagtcactgaagaagagacaactaaattggaaaagcacataatttggtgtttccaaccttaaaatttttaagtttagatttccaatctaagattgctcataatgctttttcaagtagttatgttgaagttccaggtaaatcctatgtaactgatttccttaatgtagttttgatgtgggggatgactcaatgcggattaacttggtaatcacaaaccatttagtggctcacgtctcagtatttttagttggaaagacaagctgcaagtctgtccttggaatctgacattggatcatcgtcggatgcatgttttatgatactctaataaggacttaaaagcctaagtagggtcaccagaaagctgaagcctggcaaagctacagacacatttcttccatcattaggaagagctcagatctaaatgtcaaatgggaacatacaaaaaggaacttctaggtacgataaagctaagtttgacaagttttttgtttaacctagcaccttgtagttttaaaaatcatttttagggtgtgtgcactaagaggaaaacaagttcatattcttccaccttttattgtccc

SEQ ID NO: 5 - DNA, 66001 bp, Mus musculus
  source 1..66001: mol_type = genomic DNA
ggccttggcctcactgaggaaggaagtcaccccatgcctcagggttttttttttttttttttttttttttttttttttttttggctgtaacacgaagcatttagaattaaaggcttaaaggctctggctgtggtctgagaacgagctaactgggcactgggcagagcagggaaagacagagtccctaccaccctgctggtatcctgagtggggttcatggccaaagaacgcataactgagctcaactgagctggtggtgtctctgggtgataaggaagagtcagcagagagcatagccgccaggcagtctgataagcccctcgaacacattcctggatggagtcaggggcccagcgcgtgtctggggcctggagttttgagtggagggtatgcttaaggcctcgagggacacagaattctgtacgaagggtgtgcaaaggtcccgggagacatggaattccgaggggacagtgtgcaaaggccacgagggacacagaatggagggtgtgcaaaagcccaaagggatatggaattccgagtgggggaagtgcaaagggccccgagggacacggaattttgagtggagggcgttcatggcccagaggctggcccgggctgactgcaactgattttaatgcaggggagcgggaggcattcgcaggagtccggaagaaagaagaggtcgcaggggcgggggtagtggatgcagacggtgccagggtcttctgccctctgtagagggcacatcggttcccacctagaccagcaaccaccaggaaagcccagcagctcggaggggcggcgcccaaaggaagccacgcccacgcctcaccatcagagcaccgcccactccccgcctcttcccacccctcgccggaatcccgcgccgaactcgggggcgggctgcccgggccatggcgcataaagcctctggccacctgcagggctactgctgctccggccaccgccaggcacacaccttgctgctgagggagtctcggcttctgtcatctctgtggcctccgtcacctctgtctccgtctccttcaggtcctgagccccgagagccccttccgcgcacgcggacatgggcggcagctccagggcgcgctgggtggccttggggttgggcgccctggggctgctgtttgctgcgctcggcgttgtcatgatcctcatggtgccctccctcatcaagcagcaggtgctcaaggtgagtgaagtgtcttggggagatgggggttggggatcgggacgcagggtgggacggtggagctacctcctcaccaccagggaggcccaggctcaccccagaggcttggtctgagtcaccagcgtcccaggagcctaaacctcactgaagcagaagtatggcctggttgtccctgagtttcgactgtagctgtcgcgacctccagcccttctgaacgcgccggccggtgactgtacctagtaacctcagagctgcgcgaaacccctgtacatctgtgaggctcccacgggctccagttctttgggtgctctgcctgtttcctcccgcccagatgccccagagatgccctggctcgcccccacactgcctgccagtctcgagagttaagctgctttctgccccccacgctctggcagaggtagacacgactcaggtctgccgaggataggcagcccagcctctcccttgatctcagccttaggccctgttttgccctttcaaccgggcaccgatattaggcgggtggtgccggtccagccacagactctgccctgggatccggtgtccttctatttgagccgggagctcattaagtcctggagattaccgagtaattgtgttttctgagacgcagggttactgcggaaggggaaacgggggctggctctcagggaacatccagttagaactggatctgggatccagaaggggctgttgtcacagggtttgactgacatctttcctcttagcgggtttgtttactgggagtttaagaatggcttcgtgtcccatgtcccatcgcccgctgaaaggagggaatgaatgagggatactccctcttgggctggccgctacctgccaggtcctggcgggtcttcaggatggagccacaggtggctttcctggagttgtggtgggtatttccgagttctcttggccacactgtcatttctcttgcttagaattttcaacccttgtacatgaaggtcccaggagttgccctgtcttgggtagagggaagatccttcttgttggagcatcagaattgctctgggcctttctgttaggtttggcaaagtagtggtgacagccttgtctcagtggcagcctgtggtttcagtaagaacccagccctgtgcactccgcacgtggactgggtttcctgctcagccagcctgtagtgggtagagtgagcagatatcccctgttcttcgatttgggggtgagggggaatctgagctggttccttctcaaactcctttcgggaatgttttcttttgggggttggcacagagactaaggatggggggggttggtgtgtgtgggcgtgcacgttagcgttgggactgcgcaggcctgctccatcagtcagggtcccatctgcccgtgcttgagaaagaccacgactggcagaattgccccatagctggtagcttggggcttcacgtttgagcgagcggcttccacaatcttacttgtgaaaatcccaaaggcgattggttgggggctgagtggcgtgaaggaaggaagagcctaagacacgcctagcccaaaacagcctcttcttagaggccctggctggaagccgacctcttgagggaggaaaaaaaagttcacttcaggggatggtgggaggatggcagcgaggggagcagggagggaccctggagttgactttggattcttgcgttctgtgctacttgaatggaagccttgtctttgtgtgtgtgtgtgtgtgtgtgtgtgtgtgtgtgtttctttctttccttttttttttttttttgtaagttaaagaagttgatttataaaagtcaggaatactaggttgtttaagaaaacaagttcaggacttggggtgtagctcagtggtagagaacttaccttgcattcaacaggccttgggttccaacacacacacacacaccttgcattcaacggccttgggttccaacacacacacacacacagagagagagagagagagagagagagagagagagagagagagatttcactcttattttcttctgatgatatcattttttgacataaattgtgtgtgtgtgtgtgtgtgtgtgtgtaggtcacctgatttcttcccttacagatagggtctcttgatgagctagacagccatccccctgtctctggggttacaggtgcaatcctatcgatgcacaatattgtatatgtcactgggagtttgaactcaggtcctcatgtttgcctagcgtggacatcttttacaaaacccgagttggatagtatgtggttttcttcttttcttcctcctttctttccttcctttcttcttcttttttctcctttctcttgattcgtggtctcactagatagcccaggaactcagtgcatagtccaggctggcctcaaactcagagatccacctacctttgcctcttgagtgttaggattaaaggtctgtctaccatgcctggcttaaatagtatggtttttctgttcagtgttagtattcaaaggggtgtgtgtatgcctgtgtgtgtatgtgtgtatacacgcacacgtgtacacacacatacatgcacgtatactcatgcacctgtatggacgtgcttgccacgcggtgagaccagaggttgaagtgcagtgtcttccacagtgtctctccaccttatttttttgagacaggctctctcattaaatatgaggtgtgccttctctgctagccactgaacaacaggtccctggaatctgcctgtctccgcctgtcctggtactggggttccagacatgtatcagacgtgctcagctatttttttttctgcgtgtccctggccatcctagaactccctctgtaggctctccctgcctcctgagtgctgagactaaaggtataggccaccacccctggctctgtgttgagcttttgtttgggttttgagaatcagaactcagatcctccagcgagcactttacccactgggcgatcttcccagcccctttcaaaggttttgacaggccgaacatgccaagtctgttactgccttttccccccttactgtgctgggattcctttgtttacagttcctctgttgactggccatgtctttgtctcagactccagagaacacatagcgtcctgtgtgagtccaggaacacttgcacggtctgagccttagctgtggacccacaggtgcgctctgtttggaatttggaattctctactattgctcccgctgtcaagctgtgggaccatccacttcgatggcaatgaagtgtcatttctaaaccccatcaccctctttggctacctgtggttttgatttcacatcttcagcgccacaaggttgggcaggaggaacgtgatccccgtgtaggtgacagttttcagagctcaggggctcaggcctactgtaccatgtgctagctggagccggttcatcactcaagagagccaagtttttcccccagcagtgagacaggggtcagatctggtttgctgtccctgagggattagcaagttgcaaaggagattacaagcggcctttggaaatgccctgtgagcttgcctgcttctggactcaatctaaccacggcagcgttgtgaaacaggcagaggctgcgaattcttcctctctacagataagggatctggcccatagcaaaggtcggcagctggttattaaacatggcctgtggtgggctgggagggacttcaagcggccatgctcggtttaccttccatagggaagtaggaagcaagcaagagccctctctttctgtacagtgagctgctgggcagtccaagaaaagaattaagaagggccaggtttcaggaagacatgcatgatagaaaaaaggctctgttactattaaagtccctcttccccagcctcttacccggcatcccagctacttggagtctgaggcaggaggaatgaatattcaaagccggaggaagttcagagcctgcctgggttgcacaggaagctcaggggagcttagtgagacccctgtctttatatcaaatgtaagcaaggtgcattagtgcatatttttaaatcccagcgctcagaaggcagaggcaggcagatctctgagtcaagtcaagtgcctggatagccagggctatagagagagatcctgtctcataaagaaacaaacacacaaataaataacacataaataagtaagtaaataaataaataggtaagtggaaaactgaagatagagctcactgattgatagactgcttcccagtatgtgcacatccctgggttcaatccccagcactgcaaaacataaagagaaacacacacacacacacacacacacacacacacacacacacacaccaaaatccctcagccagtcggtggtcgcagcggcggctcatacctttaatcccagcactcgggaggcagagacagagtgatctctgtgtcagccagggttacacagagaaaccctgtctcaagacaaaacaagtcaaccaaccaaccaaacccaaaatgacaagaatgacatcaaaaccctaacagtattctatattttcaacagactcgtgttagtgccaaaatggtttagaaaaaggtgcggaagggtgcgtgccagacacacaaggtagataatgctggctgcacgactctctgtacatttttgttttattttatgtgtaggggtgtttttcctgcatgtacctctgtgcatgcagtgcccttgtagaccagacaggacatcagatcctctggaacttggacttacagatggttgtgagccaccatgagggtgctaggaatttcacgaacccaggtcctgtatcagagcggccagcctggctcttaaccaccgagcaatctccaaaccatcatgattctttttgtaaaatattatgtgtatgtatttgttggtgtgtgagtggaggggtggtgctgcaacatgcctatgcaggtcatgggacaactttgaggagtgatttctctcctgctcctaagtggggtcctagggattgaactcaggtcgtcaggctgccgagccgtctcactgagcccaatggttactatcttgtttgtagttctttagggcaaggtttcgttgtattactcaggctttatttgaaatcctcctgcctcagcttcttgttcatcactgttcatcatggaagggagtcagggcaggaactagaacaagggagggacctggaggcaggagctggtgcagagaccatggagggatgctgctacgaactttctctttatgacttgctcagcttgctttcttacagaaccgaggaccaccagctctggggagacccctcccacaataggccacgcccttccccatcaatcactaattaagaaaatgttctacaggcttgcccattgcccagtctttttttttttttttaagatttacttatttatttaatgtatatgagtacaccgtagctgtcttcagacacaccagaagagggcgtcagatctcattacagatggttatgagccaccatgtggttgctgggaattgaactcacgacctctggaagaacagtcagtgctcctaaccactgagccatctctccagccccccccccccccccccccagtcttatggaggaatttgtcttaattgaggttacctcctctcaaatgacaatagactgtgtcaagttgacgcaaagctagccggcacaggcagcgtggttgacacaggtccttgtcagttaaggaaacagctgaaaatggtaagaagtaccccgtgactttcttggagtgtgtgtaagtgtcacacggagggaaacccaccagccctggaacccctgtgtgtctcacctagaacccagcctggcacgcggcttgcccgtccacagggccaccgccttcagcacagagcctcataatctttcccaagtctgggtctgcatcagctccactgtcctgacgtggcatttcctgttgcactgtgcgtgatctgttcatctgctgatgcctccctcctctgtcccatgtgtcccctaggtgcaaagcggaagcctcaggctgccctcacctgctccgttgttctttctgctacctcatacctgcttcaccattaccttttatgacccctctacccccatggccttactgcctgcttggacccgtgtggtagtctatcctgctcttctagtgagggatggtgcccaccctgctcagactctctaacattcccgtctttttctgagtgaaccccaaattcctctcagcagcttgaaggctcccaatttgtttctctttcccaccctcaattcagcaccccacccccacaccactatgctcctggtttgagcccattagacctagtccctcttcagaggccctggcgtgtccctttggtggcacatgcctttaattcccagcacttggaaggcagagaggtaggtggatctctgagttcaaggccagcctggtctatagagtgggttttatgacagccagggctacacagagaaaccctgtctcaaaacgccctatccccctaaaaaaatgatttatttattattttcagtgttttgtctgtattcacgtgtgtggtgcttgaggagggtcaaaagaggtgtcaggattccctggaactgtgacctgtcatgtgggtgctgggaaccgaactcagattctctatgggtgcagccagtgcttgtaaacctcggagccatccctccagcccaccatccttattctgtcacaggcttggatgtggctcgctctttttcctgattgagaagttggtcagccaggtgaagggctagggagctgactggatgctggccaatagctggctctctacccctccttgcctcaattctaatctgatcccttctcccacagctaggagttgggctagcctgggatatctactctgttcctgagttgttttgtgacctctggcagagcccattggcttgacacttggaaggtgtccctgatggcagatagctccttgtagatagatactgtgttacgatgctcctcagagccttttattaagtatctaaacgactacagttgctctctgcttcatctagggagagggacacaaagagagattaagtaaaacttgtcccctggtgtcccatgtctccccaccccaaccctctacctccgcattccagatttaggtgacagaaccaagatgtagcctctgctaaaagattgtaacccttgaacagctggccttgactggccgttccttctttttctcccttctcttccatcgatgtgcatggctagcgggtgtattccagggttttctcccacaagagaaggctgcagattcagcaccccttggtacctcctcaggaggactcttaagtgggaaggtctgccttttcccagtttgtaccaccctgtgaccttgggatcaaggtctgcctgggccatcgtgggctgctccccaagtggagactggggccccagatgtcaggatgggtgtccctgcaagttccctgttctctgggccctgggtaagaatgcttgggtgtgctgggatttcttctgttttcttcctctttcctgatgcctggtttccggttcccatggcttccggtccccctggcatgtgtggaactattttagttcccttcagggtgcagctgtggctatagactacctatttttagtcccaagcatttatttctgttcctttctggctcctcgtacatccacaagtcctcatagcttcagtgtgtgtgtctgttggtatacacaccttttggtgcgggtccacgtaatagtgtcccccaggtatagtagcacacagacagagcttggtgtatgtatggtccagtgctgacatccatggatgtgagtgtttctgtgtaccgcacgagctacaacctatccacatggagaaaagccaggaaggggatgcaggcacatgggtcattgtctgggcccatgtagacacatgcacatatgttgatgcctgttccatttgctgggggtgggcagtggacatggctgcatggcagaatgatggccttcgaggtgcaagccactgggctccgaccttagcactgcagcagacagaaggggtcacgggcactgcatggtcactgtgtgcagtagttaggtgctggtattttgaagccagactctcctgttgcattcctggaaggaagttaccctgggacccttcctggaaggatggattctttgctatcttcccacagtgactcatcccctcattcgctccaattcctggagttcggctaccaggaatcctgttctctggccttggatcccagcagccgactctggggccagtcctggagagagccacttggttctctgcagagcttatcctaggtgcatcttggttcctaggaggcttggcccaggctgccagctctgtggccttctgtggcttttagggtacagaggagtattcaaatgtccttccttggccagcaaaagagaggccctgggctaccgtgagcttctccccaggttgccactggatgtttgacatttcactcttaaggaccatgagttcagctgagaggccttctgaatgagagaggggggggggagagagagagagagagagagagagagagagagagagagagagagaagaaaacatggatgctcttgggtatagtggaggagaaggtttattgtagataaaagggagaacgggagaacatagctagaggcagagaccgctggaagagtccagagtggacatgaccttgaaccctgagctagatgaggggagggggagggaaaaggagaggagagaggggagccaggtgcaactgccaagaaggagggagggcaatcaaaatggttggattatcctggggagagtagcccagccctctgggctatagagttcagggtagggggcagggtatgccagcctgaaggggttctgaaactggtagggactgagggatgctgggagaacgtggcagccaggtctgctttgatatgttaactagccacctaaaccatttgtgcagggtttgaaactcaataccttcagtgccagatcctgtgccatcaatttagtttgaggaggatttagcctggcaggggtgggtgggggtgtaggtggggctctgcccaaaatagaaacaggttaggaggctaaagcagggggattgctagttctaggccagcctgggctaccctccccccaaaataaataaaaaaaattccataaaaatgaaaaaccagaaaacaaaaacacttgatccttttacctctctcctctctcctcactctctccctcactggtgaccttggcctccactggcctagggcagggtcaggccaggctgagctacaaagagaaacaacaacaacaaaaaaacccaggtctggctcagcctggctctgagtcccagtggctgaggaggttccatggctgccgagggaagatttccacccctctcctcagatcccgggctttctggcctctccccagctcccatctcctctgctttgtgatcttcagctgcctagggaggggaaggaataggagtgacctgacccttgcttgcttcctggcttttcaatgaagttccttccaccctctctggaagcttctagaaaagggtcttggctggggcctctctggctgtgggttgtctccatggttctcgtggggcaggggttctctttccccctttgcctcagccagactctggtggaatatgcccaggcccaattcacagctcagggccttaacttcccccccaacccccccacccccaccccctgctcagccccaaactgtcccctcctgggccttgacctcaaaggagtctggtgtgccaggtggctcaactttttcctgaccttccccttcaactccaggcctgtcccccttcaccttcctggttagcttggcctgtggtcttctagcttgtggcggcagcaagccacgaccttggcagcggccttccctcgccctctctgcttcccacacgtgagccctggggaagtgagcgtctcccacctttttgtccgtttcccgaggctgacttctgtcattttcttatctggatccttaaattcctttcctctgtctcttgtgttcctcccagctcccagtacagcagccagaaacagaccctaggtccttcccatgtgtcaagaggccagtaccttgccctctgatacctttctggcttgcacaccccaccccttgattcctctcgtgcgtgggctaggtctatagtctaatgtctgacatttccaagtgggactccgctgaggtcaattcagacccaatatgccacactagttacatgtacattgggcttttttccttctgccacgttgtccccatggggagtccccaggctcaggagggcaaggacctctgtggtgtttgttctttgtccccagcgttggaactgatgccagacactgggccttgggttcccaaatgtttacccaaagtggacagggtcgaatcacagggtgttctggtaccctggcactaggaagtcctgtgccaggctagaccatgggtgccgtggtgctactgtcctgccatgggtgatccggctgatccggcacttccccctgtcacagacagagtgctctttcgaagacacagtgctttagtttaaagctgtttcaggatccagctaggtgagatgatgtggatttgatccccagagcccacgtcaaacaagctggtaattccatccctggggaggcagagacaggagactccatggggctcccgggattcattcctgcaacgcaactggggagctccaggctttagaaagaatcgatcttaaaaaaaaagatagatagtgctggagggagcacacctgaggttggattggcctctggcctctacatatatacatacacacacacacacacacacacacacacacagagagagagagggagagagagagacccacacccacagaggcagacagacagacaaacacacacacccagagagacagacagacacacacaaagagccaaacacatacacccacagagacacacacatagagacatacatgcacacagaggcatagagaaagatagacacacacacacacacacagagacggaggagagacatatagagacacacacacacacacacacacacacacagacacacatacaaagacagacagacagacagacagacacacgcacacactcactcactcttgccccgatccatcagtcctttcttcacaggctccaacctgtctggccagtcagcggttctgttctaagcccacaagtctctctcctcctccattctcacacgcgccggaagttccttctgcccagatacttttccccttggctctttcactcagttcctctcctgtgtttcaaaataaatgacacgtatttgtattttggggtgcttgggagggacacagagaggcactcatactagacaagtgttctcacaccagtacacccaaagcctgtgtggggatgtttttgaagacacttcctggtcccagcttccctgacctcccttgacctttggaagtttccttgcgcccatctctagtgaactcattcctttggttgatgtcctaccaggatggggatctggtgtgttttgtacatagctttactcctgcagtggtggcctggctggggtgggaccctagcagccatcgctgaggtcacaagccttgctgactgcctctgctccattgggggggggggggctgtgcctctgttttgcttttttgtttttgtttttttgtttttgtttttgtttttgtttttgttttttttttttttttttttttggtttttcgagacagggtttctctgtgtagccctggctgtcctggaactcactttgtagaccaggttggccttgaactcagaaatctgcctgcctctgcctcccgagtgctgggattaaaggcgtgcgccaccactgcccagcaagggctgtgcctctgtctccagggggctgcctcctgatgccactacctaagcctcagaccatgagcctctgtcccctggatgccatttcttgaagccacctgggtgagttggtgttcatatgtcccagaccacacccactggcccgagtctctggcaggtgaccctagatccagggtctgagtctctgcatgttttctcttgactccagcaactcctcactggtgtctgtgccagggctgctggcttcccccagtaacctcttcctgtcccaccacaaaggcagtggaccagaaggaagagacccagttctcagccatgtcatcgggatggctgtgggcggtggataaataaatcacacctgtcttagccaagggcgggagagaccaggaggtggggtgcaggtttgcaatgcacggccttcgggagtgaacgggagtcttcagcagccttggtttgacgtggtccgtgtacagtcctccgcaggggacaagtttaggtttatgaataaagcgaagtcacttcctccccggagttccagataacctgccccagtgcgggatgcagtgcgggatgcagtgcgggatgcagtgcggggtgcagtggacagctgctttctcctgccagtgatctatatagaaggacctgggaggtgtggctcacaggtgcgaagaacaaaaataaactgggtgccattacacacctgtaatcctggcattccagaggcgaaaacaagagaaccaggtgttaagtcatcctgactacagagggaattggaggccagccctggctaaagaggctgtctaaagggttcagtgagtaatgcttgccaccaagcctgttgattggcgttcgatcctgggacccacacagtggaagaaaagaacaggctctttcaagttgtcctctgaccgctgtgggtgtacctcaggcacattcacactcctcacacatacactcaaataacaaatgcaaaaaacaataacaaatgcaaagcaatgacgttaagggctgggagaagggggcggggcttggggctgtccattgcctctgattggcttaggtcacatgctcatgtttgagccaatcagtgtgtgtcaagaaagctttctttggaaagaggcgctctctctgaggactcaggaggggatggagcgatgttctatgggttggcaggaggcaagggagacaggtagccagagtaaggacagaacccttggcccctagttagcaccggatcaccccaggttccgctcgctgcctcttatgtacgtacatgggtgctttttttgacgctggctccaggcttttttgattggggaggcagcgcgccagggtaaattttataaatcttaggttgttgggtcttcaagtacttacttgggtttctctctttttattttatttcattcttatcctcattgttattttggttttttgaaacagggtttctctatgtagtcctggctggcctggaacttactttgtagaccaggctgtcctcgaactcatagagatccatctgcctctacctccctcctgagtgctaggattagagtatttcaccacgcccagattttgctacgggctacggctgcttctgcttctccttcctatttattattattattattattattattattattattattatttgaggcaggggctcacactgtagcccaggctggcctcacacttttggcaatccttctgcctctgcttcccaaatcctgggagtaaagacatgtgccgtagtgcctggcccatctttttgtttgttaaaatcacatgtttgtttatttgtgtgtattgtggagggtaggggtgccatatgtgtactacacatgtgtagagggcagaggacaacttgcaagagtcagttttcttcttctaccattatgcatggggttgaactcaggcaggcttggctgctggggctttattggctaagtcatctctcctgccctcatgcttttgtttttgtttttattttgtttcttgagactgggtttctctgtgtagccctgactgtcttagaactcaatcagtagaccaggctggcctcgaactcacagagatctgtctgcctctgcctcccaagtgctggaattaaatgtgtgtgccatcaccgcccagcttagcgcctgtgttttatttttctctttaaggacccagatctcagagggagctgtgatctgttcaaggccatagttggtagccagtgttagctggtcgtgggccaggttaactgccaggctggccggggaagtggggtgctgggctagggaaagataagagatctcggtaagtgagggctagtcatgttgttttctggtgacttcgggcagtctcccagctccctcccacctacctcttctttgctctcatctgactgtaggagtgggaggaggtgttttgataaagtaggagagatgtcaggacccggtataccaaagttcaagctctatgcgccttctctgaggctctgagtgcgccgtaggatgcaaacagcgagaaccagcccaagcctccgcgcccttcactacactcggcatcaggctcagatgcagaggaggtaatctgctctggacgtgttctgcaatatagctcaggagtcggctcacgccccagacagagcagctgttggctcttacccaagactctgaggtacgccatgtccttctggagcagtatggggactgaagctgagcttctgctagtgtctctttgtaagccgagctgggctctctggctacccacatgtttgccgtctgcctggaggagaggtgggaggtgagtgtgttcctggagggtcactgccaaggttgtaaacccattggaaagtctctgggcttgagccttgcgggggatggctctgccaattccctgctgggtaacctcgggcaagtgccttaattcctctgtgccttttcaccgtgttcttaatagtgtcctgtgtctgaggagatcatacctgttaaacacctagaataacaagctgctgagtatcgctcagtatgctgatgcacacatcctcggggacctgtcatcaatgcttgcaactgacttctcttctacatcctgctctgagatgtcacagggcatgctcatggcacacccagctttcccaccagccttaagagtggctggggatgtctcaagactgattctgcagttgcccaagtaccttgctggctagccatgtcccagtaaactcagccaccggctgttctccctccccacgtctaggtttttcttttaaaaattattgttttggaaaaatcgctgacacaatgggacagtgggtcacattggcactcacccaaacctcttggccctgcagagaatgtgcgtagacctctgttagagtcaaggtcacttatcctcctgcccacagcctgcagctacaggctggcccctgtgtgtgaggcctctgcctccagctctgtcctttttccattcaaatctgtcttacttattttctgccagggtaggaactgaacctggggtctcctgcttgatgtgccatatccccagtcctttctccttaaaaacctttttacacttacttatttaaagattttttttttaaattatgtgtctttgtgtgggtatgtgcacatgagtgcaggtgcctgtggaggccacatgaaggcgccagatcccctggagcaagagttctagtgagccacctgactatagggtactggaaatggaacttagggtctctgcaatagcactccatgcttttaactgctaagcggtctctctggcccttatttttgtttgtttgtttgtttgtttgtatagcgattagaggacatccctagtttgtcagttcccttttctgtcatgttgacagcaagcacctttacttgctgagccattttgaaagcccaactctttcctaatattattattaaagttagtctcactaagtgtcccaggctagccttggatgttcagtcctcctgctttagcctggggtggcaggcttgcccacagagtccatatttggttgtagctgttaagtttagaggtttgtcctgtgtggatctccttcctcctatcagtggtacaaccaggcctctcttgccacctgttctgcaagccttgtttcccaggagctgatggccaccttctaggcagggcgccctggttgtggttgctatgtgctcttcccacagaccctgcacataaactgcctagggttatcctggctgagcaggcagtgcgagcagcggggcttagagtttcttgcaccagcttctgcttgagtttcctggctttcccagccccacctccatgttcggagaggtgttgatccctctgacgaccaggctgaggccagctcagggctcccacccacccttgcagctgtaggagtcctggcatgtcagagctggccttctggagttggaggcccaggaaggtgggacctgggggctcatcctgctctgctccctaccgccaccccaacccccatcctgacctctgagccctgcagaaattctaagccctggccctggtcccctcatctggcctcttagcccaggaggctcccctgggctgagagcccatcttagccagtgctccattgtagctggggtcctgacctgcgctggcctcgggatctccttctcctgtctgttcccagccctcactgtgcctcttgcgcttcctttcttattattgtgaaatagtacaaagttctaacaaaggtcataatacacatccgtcaggttaaatcagccccaggtgaaaagatggagcctcatggtcccgaggccctgctggagggagccaccacccttgggtgctccttgccttttgtagaaccatccctatatatccgacttggtctggttttgaacttcctttagtgtctacaaactctgactttattgctgtggcattttgctttcgttgctttgtttgtttgtttgtttggagaccaagcctgctctgtagctcagactaacctggtatcagtctttctgtctcagtctctggcatgcaaggcccacagatgcgcaccgccaagcctgaccacatttgcatcctggctgtggcccgtttttttcaagttcatctatgtaatagcgtatacctgtgcctcctaactctctgtgactggacaatgttttgtgtgtgtgtgcggtttttaatggtatagatgtcacattgtgtttatctgtctttagccggtggcagcaggtagctagtggctcttgtgggtgtggggagcgtggtgctgtggggtgtggacacatgctcacttgtcatgtcctctctctctccttccctgtctcctcctgtcagtgtctccctctgtggctcatcctctctctggcttcgatcttgagtacgttcttccatctgcttcagtcccatccatccacatcactgtgggtgagagcttacgttccgaggccttgggctggggtccaggtgttggctcagccttctcccagctatgtgacctagtgccattggctcacctctctgccttaggtgacttgctttaccaggccatcatcgtgaaaggccatcagctgtgtgacttaagcaacatccgcttcctttctggctcttctggaggctgcatgcccttggctgccatggctgctcctgctgtctagcttgcagccactgcctgcctttccctgtctccctgctgctatcctcagtatctctctcctagctcttcttgctgtagactccggtcacaccgcatcagggaccatcctgactaatttattgcctccttttttttttttaacctactgtggccccatcatgggctctgaatccagtacattccaatacagtttgaggcatgggggtggggtagacaggtgggagtggttcggcacctggaacgcttcaggcaatgacgctatgtggacaccttgaaatcagagtctctgtgattagtggctgggacccttgaaagactgggtcttgtgggaatgaagaggtgccctggagtagccatgcagtaagcagttgctagggatggggtggggtcacacagctgcctttgagtcatccgtgcttctataagtgaccccagtatcattgctgcaccccatctgatgggccagaatctcctcttaggtcaggtgtcactttggggtgaatagaaagagtctcaaggaaaaatcacacaagaacagggggtttgctgggcagtggtggtggcgcacgcctttaatcccagcacttgggaggcagaggcaggtggatttcagagttcgaggccagcctggtctacagagtgagttccaggacaggcagggctatacagagaaaccctgtctcaaaaaaccaccccccccaaaaaaaacccaaaaaaaaaccccagggggttttatgtttattttgtttatgtatttacttgtgtgtgtacgtgcacatgtatgtttgtgtgtgtgtgcatgagtttttgcgtatgcacgagtgtgtgtgtgtgtgtgtgtgtgtgcatacagacctacagacccaaagatgacatcaggtgtcttctttttctgtcattttctgtgggaataaaggtagattctagtattctggttacagtgtccagggaggtggtggcatatcccagcactggccaatcacttagccaggtgttaccatcttatctgggttgcccgtgtgcatatctggctcctggctccttgctgttgcctggtctccctgtttaatgtctgtgagagcttgtctgttaccatggtgacagatgccctggtttttccaactctatcaaccatatacagcatacgcaacacacttcctgggacctgccacattgggtggcttcagattggtccctgggatgtttgagtaggcagctggctgcctactgttagcaggctggttacacaagtgtccattcaccctctcagagttctgtgtgaccagccttcttgaagaagacgacctgtgggtgtttcagtttgcatttctgaggtgtgtgtgtgtgtgtgtgtgtgtgtgagcatacatctatgagtgtgtgtatatgtgtaggtgggtgcatttctgtgtacttgtggataccagagtctaggttgggtgttctcccccatgattctcaagtttgcatgtttgtttattctgaggcaagctcttattgtgtagtgtaggctgaccaggaactcactgtgtagatcaggctagcctcggaactcactatgtagatcaggctagcctcgaacttacagagatgcacctgccttttcctcctaagtgctgggattaatggcatgcaccaccacacttgatcgtaaattatgtgtgtgtatataaattatatatttatataaatctacaaaatgtatactatatttatgttatatatatttatatatataagtaaatgtatatttggtggtgccggagtttgagcccagagcttcgtaggtgctagttaagctctctgccactctcctacctcccatgccagtttatttatttatttatttatttatttatttattatttatagatttatctttagatttatttattttatgtaagtacactgtagctgtcttcagacactccagaagagggcgtcagatctcattacggatgggtgtgagccatgtggttgctgggatttgaactcaggaccttcagaagggcagtcagtgctcttacctgctgagccacctctccagccctgccagtttatttttgactcagggtctctcactgaatctggagcttgctgattgtctagactgtctgaccatcaagcttcaggaaccctcctttctccactgatcccaatgcccagagtgctgtagttatagacctgtatgtctgggtttgatgacccacttggctttttcatgggtgctggggcgagggtccgcactcgggtcctcagcttgaacggctggtacttgtgtctagagccgtctgtgcagcctgctgagcctcgctggcttgtgagtaaggctctgagaacttgaaggactttaacaggctaacctgaggtttgggcttcacttcttcagagcatttgtgttgcgctgagcatgtggctcactttgtagctgcttgcctggcacacacgaggccccgggggcagcccccaggaccccatgaactttatatgatggtccctatctgtcatctcagattctgcttggctactctagggagtaggaagccaatctgggctagagagacactggcccccaaaaaggaatctgtgatgaaggtcatggggctcaggagtgtctgcatcttgcctgggatgacagtttgatagaggccagcagaaacgtcccgccaagggctagaggccaaggacgagtctgtcccatcaccagagctgaaatcagggtgtgtttctttcagtggtggtgccccagcctcctagagaggcctgtgttagtcacctaggtctaaacttgtgtccttaagccgtgggaacttcatggctaggctgggcgcttctagtgtcttgagggctcttggctctctccattcctggaggtgttcaccgttcctcagttgacggcatcaatgctcctgtttctgtctctatctctgtctcaacgtggcctcctttgtattttccatttcctcttcaaaactgagttattagtattatttatgtgtatgtgcatatgcctgcatgagtttatgtgcatagcatgcgtgcttggtccccatgaaggccggaggacatcagagcccctggacctggagttacagatagtgtgagccaccacgtagctgctgggaaccgaacaccgatgcctgcatccatcgagccatctttccagctcccctgctcttctttgagacccaacctaagtccaaacactttgacattcattcctattttgagttaagagtctcaccgtgcagcccaggcaggctttaaattcacaatcctcctgcctcagcttcccaagtgctgaagtgacaggcctgtgccactgcgctgatctgatatttttaacttgacatctggccagaccctatttccgattaaggtcacacagggcacagtcaagtatcttaggatcaccacctacatagatgcatggcagagtaccttttgcccagacagcccctactgtgaaaataaactacaggacaaaaatcaccagtctgtcctgatagccggctctcagcacttgaaggtaggaagcaagaggatcagaagttcaaggtcatctctgctaatagtgagtttgaagccagtccagaggggaatgaccctctgggaagacaaaggtgacatgacactcgcgtcccccagcgtcccaagcacagaggtaactttggttcatggctctttgttccttgctgcaaaaaacatgtttgtgtttagaagagcccctccctgccctccctggcctccctctcctggcccccggtacccgtgcgcacttcctgcactttgcagtctcggattgggcactcagatggctctgggtctcccccttctttccagagcgctggagggatcagccgtgtttcccatcagccaaggccgcggggaatggctgtgcattcaatagcatgtctctgggaaattgggcctattccagcaccatcttcccgtgtcttccggagctttctcaggagatatatttgtttctacagacgccactaattactcctggctgggtgcctggaaatcacatctgtggattaaggtgccaccagatctgagcttcctctagggccccaggagtcctgacatatcccccattcagcatacttgctcgcttgcttgcttgagctcagtttgatttctccactcatacagcccaggaccccttgcctagggaatggggccgccaatggtggactaggtcttctcaggtcagataagctaatcatttagacaatccccacaggccaatccaaatgtaggcaggccgtccttcactgagaccctcttcccaggggaatctacgttgtgtcaggttgccagagctaaccgctgcaaggttataattttaaaggacaagacacatcattgggcgtgcttagctcaaagttgtggcttaagtacgtaaaagagtaaaggttaatcaacacagaatttggtccccgaagtcacttgccgtgtttagtcaccctcagagcttcacagtaagaacttttctcaaagaaaaaaaaaaaaagaacagccgggcagtggtgtcgcatgtctttaatcccagcacttgagaggctcatatttctgagttcaaggccagcctggtctaccctccaggtaccatgctgtgtgaggtccaggcaagctcaccagatccagatccgctcccatctactgaggtccactcaaggcattttctgcaagtggaggctcacaggcactgaggtagaggtacccgtgtgaggccagatgagtgaaccttgactcaaatccccatgtccaggaacttgctgtgcatcaagatggcaggatttatgtggcagcccctggcagagctgtccccttaaggatcttgatctttagaccatctcctctctggatggtcatctctggtgtgtggctgagggcaagaacttgattccttgcgcctttggtttcctgcttgtcagtggggctggggctgagagagataaatgggtatcattggggaatcagagaggtgttgccgggatcggatccagctgctggctggcatgggtagcaaggtggccaattcaaatcgacagtgaagagggccagagagacggctcatcacggggaagggttcttgctgctaagcctagtaaccggaattggatctctggaacccacatggtggaaggacagacagactcctataagttatctatcctctggcttccacacgtgtgctgtggtgcatctgtgcctgtgcacataataaataaatacacaaataagtaaatgtgatttaaaaaaataattgaaagggtctaagaagtgtcctgtgttttctgtaattctccactaagtttatggccgatggccgtatggagtagaatggaggaggcgggcccaggtctagcttccgactcagtcagggtctcatctcccaaggactcagtttcccggtctgcagagtggttggtgttaattcctgtatccagttgtcaggacttaggggtcagcccacctacctataaggctgttgtctggggtgggagtcattgatgatgatgaaggtgtccccggggacctctcacacaaggggacgagtcaaagaaaggtgaggagaagaggccagccagggaaatggccatggctgtgggcgggaggctgcagtggactgagcagcctggccttcgaggtctccccacagctgaccttcccagtctgggctttcatggtgtgtggtccaggagcagataagtggaccgagctatcagcagtcagggtcaccgagtcctcatgtggaagtggaggcgggggagaaggggaagggagatatagaatgggctttgtttcaaggtagaggccaatggagagccattgtggaagaaccctatatgtatagcctggcctcagagagtgccattgccccctacccagacccacagcggggtctagctgagctctggctgaaaaaaaaaaccaaaaccccccaaacaacaataacaacacaaaacctgtgggattggaaggtgcctgtctcctagcagctgggatacccacaaactccagctctaagtgtcgatttaagacacttggtgccttaagtcacctccaggtgtctttcagtcccaccaaaaccttggggattattgatctggcagatgggacagacacagaggagcagggtagagaggaggaggagtctgaggctcttctggggaagcaggtgttccctcttcctgttgagctgactgccccgttgcatgctgggcccctcccaggccctggagttgcatggtgggcagagaaacagggtcttgatggactgtctgcaggacttgacacctgcacaagaggggcagtgtgactggcctgtggtgagtgtggctaacaaggccactggtgagggaggggcctctggtgctgaggagaagcaggcccctctgagggccaggcaaggagcatttccacctgacagaaaacgaaatgagtatccgaagtagtttacaggtggtgagagccctggagcagatagatgctcaggacacctgtcactgggctgcaggttgctgaaggtcaaaggtcgtgcaaacccacaaggacagcctctgttgaacttagtaggtcgtaggtgatgttgctggtactgtaaaagggtgcagctgccatgagaagctggtgtcaggtcactgtcccgtgcagccgtcctcctggggagtctggacaccacacaacagctgaaggtagaagggtcccaatacttagtgatgctggatgggcagataggaaaagcagatgaaatacagagaggtgagctggagagggtttcagaggtcaggagctcacatggctcttccaaaggaccagagttcagttccctgcacccacatcaggcgagtcacagctacttgtaatatcagctccaggagatctgacacccttctgggagcctctttagggactgtgctcatgtgcacagagcaacacacacattaataaaattaagattaaatcttcagaaatgaaaagagggctctccaagagggaatagtatgcagccgtataaaggaagagagagattccatttatttatttatttatttatttatctatctatctatctggagacagggtttcattatatagtccccaggtaaatagttcaaactcgtggtcctcctgcctcagctttaaagtcctgcgatagctgctgcagaggagagaattctcccacacactggaacatggatgaatgctgaagttagattctgagagacggcaggatggttgcagcgtgatttcgtttactggggcccctagagtacctattgatgaggttggagaaatgggacactaaggaggtagtgggaggggaaaggggccagggagagtgttaagtggagacagggtctcagaaggacatggaagggcgtggccattggtggggacagttgcccaagccatgggtgtacttaacaccatcagaatggatgctataaaatggcactgcctttaatcctagcagtggaagcagaggcaggcagatctctgagttcaagaccagcttggtctacagatggcgttccaagacagctgaggctacagagaaaccatgtctggggttgggggcttggggggagagtgacaaaacaaaacccaagtctggagactttagcatgataaaaatgaataaatcattatggctgatgtcccctgaagggacatgggtcacagccctccttactctggtttgtaaaagggggacccagtggccatagtgactatgttccagcctgatgagtgtcttagaaaaaacccagttccctggaaatatggccctgagctgatagctgatgctggctgccactgcccttcctctgaggtttggcagggctctgggattgttcttgaaccccagccagtctacagctggaggctctgtgatatgtcttccttgaagtgtctgggagttggtatcttcctagggacagccttgtgccccaggctattgcatgcagtgccactggaaggagccctaaggggaccaggcaacagcctggggattaggctgtagagacctgaggatggggcccctggccaaccccaacggccttgtgtggcactggctcaatggagaactcacacgtttctaatgtctgtagctgctactgaaagcattctctagaagtctcccaaagacttctcactcagtctaaagtcagggctaccttccctgtatggttgggctctacccaacaggtgaaccacatgtgtttctagaggggtaggaggcagagaggccttgggtgggcaacaaggtaatctgcttctgtgtcaatttcttatctggcatgtggctgtcgagaagcagagaccggaggatcatgggcatgagctatacactgaggtccaggctagcctgggtgacataacctgtcagaaaaactacaagaaaagattcgcattcaccatctggcttggaccttggttaagtgataaccattgtcattagcctgacacttaggcgccaggatcaagaaacaagaactcccagaatcctttggggcaccagaaagcctttcattttgagataggctctggctgtgtctctggctctcgtggaagtcacacagcaatccctttgccttagcctaagtgcagagattgtagacgtgagttatactagcctcctctgcatggattggcttgacctaaactgagctgggctctccatcgacttgcttctgcgatttcagagtggcagcctgacctagctctgcagtggcttggtgcagctcagcctgaggctggtgagatgcttacaagtcatagctggttgctgcttggtgctggtggtcatgttatgagcaaactggtccctggtcagtgggctccggattagggctgtgatggaggcctctaggaatctcgctaagtctctcactggttgttttcatctctctgagcgatgtgtaggattgagtgacagatggcagataattgggagtgggtgacaggcttcagtattctactttaatggctgttacccggtgatgccggcataggcgtctgatgctctgagtacttcctctcatgtccctgagtcatgcagacttaggaggcctcttctccactcacaaccgtttccctgagggcagaatccagttcggttaactttgcttcgtagtactaaataccagtttggggcctcgacagtgcgggggagtggggcgggttagaatgaaaaaaaaaggagcaaacaggttcattaacagctaaatgaatgaatgactgatgaaaggatgaatgaatgaagtgttctcagcctgtgctgttctgggtcagggctgagtgggaatgtcaaagggtgtggccactatgtgagccatggagttggggttagagggaaggctgcctgtgggctgcaccagtgttggggtgcaacccttgttgggcagcacctagttttccaaagggagtcagaaacgaggcttttgagactaaccttggttatgtctgacagggtttgggggtggggtgggcacatagacactcttgctttttgtttgtttccttttcttttctggggagtgtgtggtgtcaacttgacagggtctagaatcaccttggagacaaatctctggatgtgtctgtgatggagccatcctaactgctggagcagagaaaaaggagaaagatcctagcagtcattcacccctccctgcttcctgactgtggacacagcctggcaagctgcccctggcttcttcctctattgcctttccaccaagatggactgcggctttgatcagtgagccagattagccttccttaagctgcttctgtcctacgtatgtatgtatgtatgtatgtatgcatgtatgtatgtatgtagcaagccaggggcctgtgcacataccacacaagtgctccgatgttagcatttggcccttgagacttgagcgttggagtttctatcgagtctgggaaaattactgcacaccagaagcctgttaaacagtaacaggccccagagcgagagccaacctcacacgcctcccgtaagcttctcccactgacagctgtctgtgggccagacacggttgatatgcgcacctgctccctggtcagcatggtaggggttctcctctgagggactttattggggtagcccttgggagcagaaggaatatggtccggggattgacatacacataggtgcccacgtgtttgcgtccagtccagtccaaccagggagcttcctaagaaccctggagtggctctcactccacttgtctccagggaaactcactaggtcctgatgttgccctagaggatgctgggaccttctgatggagccaccttagccctctttcctggggctctgacttttatagctggacaggctctctgctttgtcaagctggggtcaaagtgaggtcacaaggcttataccttgaggacacctttctacttctgtccctaagatgtatctgcctgctctggtgtgtcaccggagcagaaaccagtttccccatagcccgagggactcaccggccacaccctttgacgtctagggatcatttgaggatgagattatgcatctcccagccactcaatcggacgtcggcttcacccttgtgtattaagctactgtagcaaaacccttgaaaacatttatggataaccagcgggcaggtgctggcgtttccccaggatgtacagtgaacaggtacttagctctagcaggcaaaccatggcgccgacacagttccaccgacatcgtgcatgcattctggttgccccagagccttgccaacacttgtgattatcaatccgtctccttttggctgtgctggtatacgtcatagagattcttgtgaatttgggggatatgtccagagttggggagggctttatgatgtcacaggccaccagtctttatgctctgtcatcctttgggtatggttttcagtctcaaagctttcccatgatccaaggtagctctgtagctctctccatcaggaccgaatttctgggtgggggtggcgggtgtttcactgaaaccaccggaacttttagtgtggtgggaggggctgctgttacaaagattggtcctgtccattttcttttttctttttttaagatttatttatttattacatgtaagtatactcagacacctacagtatgcatagggcatcagatctcattacggatggttgtgagccaccttgtggttgctgggatttgaactcaggacctttggaagagcagtcagtgctcttaactgctgagccatctctccagcccttgtccattttcttccctgctgtggtttaggcctagcacacagcctcttgtgcactgggtgctcagcagaggcctcgtggatgccaggaactgaacccaggtcctttgtaagagcagccagtgctcctaaccactgagccatctctccagccattcctggtgtgttcctggtgtgtggtgtcttcatgccctaggctggattgtcctttggggcccatccttcctgtatacatagtaactctacagggctagtacattgcgactggctggctggctcagagctgacaggatatgtgtctcaacccagatccagggctggtgaacccagtggcaatgaccagaactgtttcctctccctcctcctcctccctctcctcctcctcctcctcctccttctcttcttcttcctcttctctccttcctcttcttcctcctcttgtttggagatagggtttctttctgcatccctagctgccctggtagaccaccaggttagccttgaacacagagatccacctgcttctgcctcctaagagttggattaaaggtgtgagccgacctctgtcagcctggctcaaaaacctggtttttttttgaacctgaaatgtgtttgctgtgattcttgggggaggagggactggattttgcaatttcccaggtcagattaatgattgctttgcgagggctgcagcacctgagctgagccacactgagcagggcctggagaggctaggtacagtgtcctggagatcctgtgcagtcctgtgtggggatagaggctgggggagcttccatctctgtgatcttcatgaaccagaggcctctgagcctgatgcctgtgcacctgggatatatggtcagagagttaaagaggtgtagtgaccagaagtgtcctgttacccacacagagggtctgttgggtccctagcacctcatgtggcggccttgatcagctccagctgcctttttgtttgtttttcttttgaatggagcctctgatgtccaagtgtgttcggttcctgcagactgacctatatcctggccatcagagaacccgctaacccccggattatggtgctcactcccagtttggcttgcccagaccccacctcagactcaaagcctttgtttctctgatgcccagccaccctacccaatccctctctggaaggcagatggcttattctgtgcttattctgtacccaggtcactgctgccaagcacatgcctagtatgtactggggtcgtgggggagcaggggtccaagcaaatgatcagaatgctcacagacctttgtggtgtggagcatggagattgctccagttttcctgtctctcgggcacttggtgtgcattttggacatccccaaggcttgcagaatggagtcgaggtagcgtatggctccccacggaaggaggaaacctcagtgcattagtctgtcggtattgctgtaatgaaagacacaggcttggtgttagcatcacatctgatggggccacttcatgttgttgcgaactgcttggagcagctatgagccgagagattacagtggggaagccagaaggcaaagaaagtctgcaccatccccaccaccaatccccagccctttcccagtggggctaagcctcctgagggtcccctcccaaggcttcatgtcagagaccaagtctccaactgccaccgaaagcacgagaataaagtgtttgcagagcagacatttttaaggtaatgatctggtgggccaatgagtgtcctttagagaagacagaaaagaatgtgcagtggagaggaggggacagagaagcaggggtgggagccaagaggctgtacgaggctgagaggctcaccgctggagctcagggatgctttggtgacagctggcccaggaagtgggagctgtggtcatttattttagaaatataaatgtgtgtgtgtgtgtgtgtgtgtgtatctgtgcacatgtgcatactcggtgactttggaggccagaggatgatgtcatatcccatggaactagagttacagatgattgtgagccaccaagtgggtcctgggatttgaacctgggtcctctggaagaacccagccaatgctctaaccaatgaaccacctcactttgttgttcaaaggcagggtctctcactaggatgtgagtctcgccatttttaggctagcctggctggccagcaagccctgggcattatcccgctgtctccacctctccagagctgggatcacaagcatacaccaccatgcctggctttgtatgggggccaaactcggctccttccacttatgaagcaagcactttatggctgagccatctccctagaattccaaactctgttctcaagaacacactatgtcaaaggtgcccacaggatgctgggggtttgtttcctacaatgatggattatggcgctccagaattggcttgggctccttcctccaagtcctctgaggtccttccaggtgcctggcactgctcagagttcacggatccagcatgagcatcattggtggggtttatggcctggcagagagacgtgagcacaatacacaggatgagtcattaacgtaatcgggaagaaggcagggagagcctcggaataaacaagagtttaagagggagcccagggctcggggtgggctctgtgggggttcctgagcacagacacaggatcccaggaggcttcctctgcaaacatggaaggcagaagaagcgacgaccagtgcaagggcctggtgggtgagagtgtgtgaacaggcaaggcagagcaagcatgccaggctggtctaaaggactaatgggggtatcagtaagacacagccctagagatgccactgagccagatgggcaaaggcttataggtcagtgttagcatggtaaaatttacaataaataaataaacaaacaatccatttggtttggggatggccatacatatgccagggtgcatgtagaagacacagaacaatgtcttggagtcttctgttctgttgtgtgggtcctggggatccagctctggtccttaggcttggtgattaagctcctttattttgcaggccacaatctgaaagatgctgggccctgggcttcagataggggaaggccactccccacaggcattccattccccactctggtggctgtacatgaggtacctcgtggttcagcctcccgcaacaccccaaccacctgcttgcagagggaaccgcccatcaccccatggttttcagtgagattctgcctagtcctctgtgccaaaaaagccaagcagaatctttggggaggctgcctttcctgctctcctgagagttctctgggtggcgaagggagacaatctgtctgagatgaggaccttcgggcgctctccaccgctctgttggcctctcaaaccttgagaatccctccggggcagagttttcaaactgggatgttatctttctgatatcggttgtgacatccctggggtgtgtgggcaactgatgtttaaaacatggcgggagtgtgctggtgatgatggggtgataaagaaatggctcagaggttatgaggagtatgtctcatctcacacacacacacacacacacacacacacacacacacatacacacacacacacacgcgcacgcacatttgagtgtggggtgctaggaactgaattcagtctcctggaacagcctcaagtgcttttaaccactaagccagctccccacccccaccccacccccgcaaattaaaaacaagcaaatatataaaagtctaaaacacagtgtgagccaggtgtaatgataccccaggctccctataatctggacattgaagaggcggaggcagaaagatcaggagtttcagagtcatcctcagctacatatcaaatcagaaatcctggagttggggtggaaatcattttgtactggagaggttgccctgggtggcaggcagcatttgggagctggtattcggggtgttgtcccaggtagaggctccgcagagccatagggcactgcacagatatgatctatgttagaagtgcacacaagtacccgctggctgcccgggctgtgtacctcacccatgccctcacttatttagcttcagcttcctattgcagtgttctggggaagtatagtctacttgactttacagctgaccttttaaaaatgatgggtgcatttcagtttcggtattcaatggagggcgcagcaagatggctcagtgggtgaaggcacttgctgccaagcctaatggcctgagttcgatccctggggcccatatggtggaaggagggagaactgtcctctgacctccacacatgcaccctggcgtaagcaaacccacacacatacccatatacacaaataagtaactgcagagattaaaagaatataaaaacctttattgagacacgccagccttgtttattctggaatttctcacagtaggactcacaacgccctgtgcatatttccttctgtattctctcccccagtagtatactgagtccttccccttcccctccctttttctttttgagatggtcttacgtagcccaggctagccctgaattcactatgtagctgaggatggcctgggactcctgctcctcctgcctgtgcttctggagggctggggtggcagaggtgggcactgtgcccactttgttcagggctggggatggatctaatttaatccaccaggtcacacacacacgagtggagccatgagcacccgtgggacttgtctcgtgttggcagaggcaggttggtaatctcccagcagctccacagatgtttcctagagtctcggtttataaacactcctgtcatgggtggacacggagccccgcacgcttgctcggcaggccagcctttgctggaacaccctcctgggaccttttcctggcaaagcctgttttgtctttgttttgtcctcttgctgttgtgaagcgggtgattgccatgcagcaaggctggcctcagaaagccagtcaaccctctgccttagctgcctgagcgctaaagtgactggcgtgtgctccaacagcgtgcaccccgagagccagctcttaatttttttttttttttagttttccaccatgtgttgatttggttgtggtggtagggattgaacccagggccttgcacgggctgggtaagctcttttccactgagctgcatctatgtcttctcttcttttaaatgtaaaatcgtttattcagtctttgagaaccacaacattcatacaacgtatttcaatgatacacgcttgctactcctcccagatccccctacaggccactcctgattttatatccattttggttttggttttggttttgttcaccccttcgagacaggatctcactagatagtgctggctggtcttaaactcacatagatccttgtgcctctgcctcccaagcgttggaagtaaagtcacgtgccaccacagccagtactttcttcttgtcttttctttttcccccttatttacccactcaatccaattgctgtggcccatatacatattgtaggggcagttttacactgtgtggcagtaagcaaaatagtagttctggggtcatgggggaacagcacccccagagcctgtctcagccatttgattctcagccagatttatagtatcgggcatgtttttcgtcctatggagaaggccttaaatccagtcagagagtattggctaaacccataatatccgtgccactaatgcgtccgtgggcagatcttaccaggctgataagctgcagggtagtttgcaggagtcacagctgcctaagggtgccagggtcttgaaatgcctctctgtttttgccagatgttcaggctgtgggtgaccaacggaaggtgcggcaggacatctcttaggtggactcagcaggggatttttctttcagtgggtcccgaggaggacaggccagacccagccatggctttcatgactccggccttgcagtactcaaggacaaaccccaggcccagagagccaagctaggcctgagagagtgtggcccagtgtccgcctgcctcggagtctactctgacgtggggctgggcttgacctgccagttctggcttgccacctcccctggagcctctgcctgttgtttctggctgtttggggacagtggggtggttgtctttgtcctggcaggtcgagttcaaagtcaaagctgtttggcttttggacactgctatctctgatgagctgggggtggtgactcttagctctgtggcattagcagaggtcccaggtcagggaatggtccacctcttcctcctagccttggagaaaatcagtagttctgttttgagacagagtgtctcattcactgtgtatcccaggctgacctcaaagttaccatctaactccttcagtgtcccaagtgctaggatgacagccctgcatgactggacccagggctgcctgcacactaggcaatcactaagccccgcccccggtcctgcttactgtcttctgtccctcactgctggtgactcaagaatcatcctcttccctctccctgctcctcagtacatcacacagcactggctctgcccccgctccacccccccccacccccacccccccacccggtgcataaagcccttgtcttcccgatttggtaaaagcttcacgggactggagacagccagcctgtctacttcccactcctgtctgctactgcgggaacctcatctgtcaaaaatgaatgaatgagtgaccaaatgaatgaatgaagaaaagaacaaaagaatgcgggagtaaatgtgaaatgatcagtgaataatgaatgaatgaacgaacgaacggctttctggatgagttattgactgaaagaatggagagacagccacctgtgcagagctccagccctaggagcacctccctcgatgagcactacctagccagtgttctgttttgctgtgaagagagaccacaacgaagacaactctgtattataaagcatttaactgggggcttgtttacagtttcagaggcttagtccgttatcctcatggcagggagcatggtgcaagttaggtggagttagtgttggagcaagttagtgttggagcagtagctgagatcacaggcagagagggagagagagagacagggagggagggagaccaatggaccctgtgcttggagtgagcttttgtaacctcaaaccctgcccctagcgacacacctccttcaacaaggccacacctcctccgacaaagacacgcctcttcctccgacaaggccacacctcctccaacgacacaccccctaatccctgcccatagcgacacgcctcctccgacaacgacacacctcctaatccttccaagccgttctccaactaaaggctaagctgcaaatatctgagcctgtggggcctttctcattcagaccaccccagacacccttccagatgagccttggagggtccatggcgcaggccaagtctcaggcagctgttgcagagccgtaaagtggggaagcccctcctcacactcctccctgtgtctccccacagaatgtccgcatagacccgagcagcctgtccttcgggatgtggaaggagatccccgtccctttctacttgtctgtctacttcttcgaagtggtcaacccaaacgaggtcctcaacggccagaagccagtagtccgggagcgtggaccctatgtctacaggtgaggccaggcagggtggggtgggactgtgtgtgggtgtgtgggtgtggaggggggtgttctgccatgctgagttttggagattgattgctccagagacagaagtcggacagacaccgcctctccacagactgtcagccacacagagaagccggaggccacagtggctgacagtctgtggagaggcggtgtctgtccgaggcgagtgcatactcgcatgcgtgtcctggggtgagggctaatgtgagtgtaaatgtgttgtcatgggggcggggcacagaggacccaacagcgcacaaaatgggcaaaatggctgctgttgagtcagaaaagtcaaacaaacatgcagaagtgaagccagttacgcagcatctaagaagtgagcggtgctgggtagtggtggcacacaccttgaatcccagcacttgggaggcagaggcaggtggaattctgagttgaaaactggatccaggagcggctgcctggtcctccaccttggacagagagttttaacttccataatgcctattttagccccatctgtgagacagaggtaatggtacccactgtgggtaggttcaaggataagatgagatagttagttggagccacttaagctctgtctgccatgccataaactctcagtaaacgctgcctgtgtggatatctgctccatgctggttgtcaagtgaaagggagacgtggggggtgggggcagcagttggagttgttttccagagaggctcaaagagccttatggctcaccagggaaagcagagaagagctgattggtggtggcagtgtcttgccctggacagacagcgaggatataaacaagaggtggcaagcttgatttggtggccttggtgctgttgggtatcagtgtaccatcaccccagggcatgctgggagatgatgggtgctttcatgtgacctcacagagccacagctgactcaatgcctttttacagggagttcagacaaaaggtcaacatcaccttcaatgacaacgacaccgtgtccttcgtggagaaccgcagcctccatttccagcctgacaagtcgcatggctcagagagtgactacattgtactgcctaacatcttggtcctggtgagactgtggccctgtgtcaaccccatgccaaccctgcttcctcccagcttagccttcaggagtcagagagcaagggccacctcagccccactgtctccatggtgaccatcccctccttctcattgccttacctcccatctgagactccgggacctccactgtggtccctggggaagcatgaaggtcagagccactgggggccagagggcaggtgggagtccggaagagcaggcatttattgagcacactgtgtggggtcgctgagtctgtcctttggtgtatgcggggatctactcaaccctgctgttcaggggaggaagcaaaagccaggcagaggcagggaatttacacaggggccagaaggaccctccccaaggacaaaatcccagataccaattgggccagtgttaccaaggcaggatcggatcctgtggtcagacatcacggccctcgctggaagtttgaggacagggtacacggtggttggtagtggggggacagtcaaggcatgatgtcacgaggtaagcattaccattcccagtgacactaacttgagagattggaactgtgagctttgtgtgattttcatgctttttagaagattcttctgatctccccccacccccaccctcaaccataggaaactgttgaaaacaccagctttcctgcagttgatgcgggccgatgctgactggattggtcctttagccaccttgcctcgagtccattttcagaggtgtcccccgggtccgatggggctggagggacactttttgtttttattaatttattatattactactatcattatcattattttgagatagggtgttactgggcagccatagctgccctgtatctcattatgtagaccaggccagccttgaactcacagagatctgccagcctctgcctcaagtgctgggtgtgagccaccacatttgtcatattaattccagtttttaaaaattatttattttatgtatatgagtacactgtagctggcttcagacacaccagaagaggtcatcggatcccattacagatgtttgtgaaccactttgtggttgctgggaattgaactgaggaagtagtactttaaccactgagccatctctccagcccttaattccatttttgatattgaaaaagcaacagcattctgtgctgggccagcaggatggctcagtaggtaaaaggtgcctgtcactaagtcggatggcctggctttaatctccaggacccacatgactgcctcacattgtcctttgacctctatacgtgcactatagcacccctcaataaatataataaaagccctgtgttgaaggagacctgtaagtgttggtgggtaggcatcatgcgcttgcttgatggaaagaacctggagacaccagccatgcgttgcccaggaggtggaaggtggcttttggttgaatggcctttgggaaaaaacaagagcccagatgctccatacggaggaagaacaaacaagaaaactataaactcaactgaacttggggcagttagaccctgggggggcttcgggggagtagatattctgggctcccagtgttgaggtggggcagggacatgagaagtgaggtggaatccctgtgaggagactgcggcaggagaatgctgatttccaagccagcccagattaatcattgagaccttgctgtaaacatgttcaaaaggaggccacggggagtcggggaggctttctccatatgctgtgtttctaggctcatgttactgatagaatagggttcacgtcctagctataaaccgggtatccaaggggctggcgagatggctcagcgggtaagagcactactgactgctcttccagagatcctgagttcaaatcccagcaaccacatggtgactcacaaccacctgtaatgagatctgacaccctcttctggtgtgtctgaagacagccacagtgtacttatttataataataaatgaatttgggaacaagaagaggccctaaaaattcaattcccaacaacatgaaggctcacaaccatctgtacaactacagtgtagtcatatacattaaaataaataaataaatctttaaaaatcaaaaacaaaacaaaacaaggtattcatatctttgctggcccccttctgtgttaggttttctcttaaccttggctccagcctcatccgttcgcttggtgagggatggcatgggagcctccttctctgtgccccggctctgcggagtgtctctggtggttcactggttccatttaggcctcaggtgtggctctacagacacagtggctcaggaaggcttccggaggtcaggcctgagcttcgtgtatgcctctgagtccttgtagagttcactcaatctgaacccatcatcgggtgttctcgagagtacaggttaccctggcaacaaggaccagagaaaggtccatcctgagggcctgagctctttgcctgaagcctggttgaaagtgggggatggtgtcctaccatgtcctttaccctaatcccatgtccttggctttccaggggggctcgatattgatggagagcaagcctgtgagcctgaagctgatgatgaccttggcgctggtcaccatgggccagcgtgcttttatgaaccgcacagttggtgagatcctgtggggctatgacgatcccttcgtgcattttctcaacacgtacctcccagacatgcttcccataaagggcaaatttggcctgtttgttggggtaagtgtcttctgtcccttcagagagtcaggttatctctcaaggaccctaactcaaaccagcttaaacaaaaattgggaaattatcagctcatataacaggagatgccaggattacattcaggtgcggcttgatccagttcgagtggggggatatggctggggtttaacttagaagatttgaggcacagcttgcggggaggaggaggaggactggatcttcgaagcacacctctcaaatgtccccatcagtgtgacttagaggcacccactatttctcacagttgacctttggtaacctgagctaaggtttggtctctgacccctgaccccaactctctggttcagctcagctttctctgtggggatctactgacggggtgtgcttgcagtaacgttacccacaccctccaggacctgttattactgagttcacgtgagatttactacccagtggttagagcactggctgttcttcaagaggacccaggttcgattcccagcacccacatgacggctcacaccccatctgtagctccagttccaggggatctgacaccctcttctggctcactcaggcactgcagtgcaggcaatgcacagacctacatgcaggcaaaaagactcagacacataaaataaaaatatgtccaatcttgtggagaggagacggggtaaggcactgtgtctcccagtgaccagatggggaccccatggggtgaacacataacgagggcggcaggctgaatacccagtcttaaagcttttacagagttagaggccctgggtaaagccctgtacctgtctgtacctgcccttcccagtaccccatagagactgccatccctctcttcctgacaattgaggtggggcccttctactgagcttctagatcgttcctgtcagaaatttgtattaaattctttctttctttttatctttgttttattatttttattgagtcagggtctctctatgcagtcctggctgtcctggaattcactttgtagaccaggctggcctcaaacagatgtctgcctacctctctctcctgagtgttgggagtaagggtgtgtgccactaaacccccgctaaaatgcctttttactttattagctgttggccctgattgagtagacttgtcatctgtgtacatgggagtatgaggcaggagtatggcaaattcaaaaccagcctggacaaccctgtctgaaaatgtgcaataaaaaagaatgctggatggagggtggagctggagagatggctcagcagttaagagcactgactgctctaccagaggtcctgagttcaattcccagcaaccacatggtagctcacaaccatctgtaatgagatctgatgccctcttctgatgtgtctgaagagagctacagtgtactcacataaaattttttaaagggtggggggaagctgggggtgtgactcagtggatgagtacttgcccacatgtgtaaggcactgggttctgtccccatcatttatgcagcaacagtgaggggacatacctatagctgaagaataggtgggcttgtgcctatgttgtgatctatgcccagggctaaaggtgctgttccctgcctgtctgtagatgaacaactcgaattctggggtcttcactgtcttcacgggcgtccagaatttcagcaggatccatctggtggacaaatggaacggactcagcaaggtgagcaggagggcagacagtccccatcgattggtgtggggactacaccagaacaagccttggcagagggtgtccgggtcacccgaggacttcacggatcccacaactgtccctgcggtatttctgtcgggaactcttttctgttcctgagttgtcatttctaaggctgacaggaagacattcccataaagataagacaatgaggtccagcaccttgcctaggcccacccggaaatcccccaacactgcttatagaacccagcttcctggccatttacccaccaccacgcctctgtgtacccagagaaatgtgttttccttccttcttcgaatacacagaaatcctgtcatgtgctattttgctgctggcttttctccttaggggcatctttatggataatgtgtttttgttctgttgtgttgtccagaggcccaggtactaaagatggaacgcaggccatgtgaatctttgacaaggacaccactgctgagctagacttctgcccttcactgggggaattctaggcaggggctctaccactgagccacgcccccagcccctcactgggggattctaggcaggtgctctaccactgagccacacccttaaatttttttattctgaaataaggtctctgtgtatcccaggctagcttcaaaaattcaatcctcctgtctctgttgccttgtaagtgtttaattcatagatgtgtgttaccacacccagccaacagctattcttagtgggtgctgttcacattaaccagctgctgttttatatgcattggcattttgtctgcatgtatgtctgtgtgagggccttggatcccctggaactggaattatagacagttgtgagctgccatctgggtgctaggcattgaacctgggtcctctggaaaaacagccagtgctcttaaccactgagccatctctccagcccctaatcgtctcctttctactatgagaagggctgattgaaagctcttacggcagagtgggaagtgcgtttagttttgctgttgctgttgctgttttgttttgttttgttttggaagcagggtctcagtgtagtcctggctagcctggaactcgatattgtacaggtgacttaactgagtccccacaatactgtatgttctgtccccactgtggggaacaggaagctgggctagaacctctgtgcacacagcctgctcacgggcttggatatgatgatgtctgtactagggcacacattgggtgtagatcttggctgtccctcaaggctatatctcatcctgtcctgcagatcgattattggcattcagagcagtgtaacatgatcaatgggacttccgggcagatgtgggcacccttcatgacacccgaatcctcgctggaattcttcagcccggaggcatgcaggtaagccctgtgtagggactccctgcctcctaccaggaaactctgcttctgagatggttcagggtccactcaggtagctccctggaagtgtgctcaaagtgtctggccttctgtgtactatgcctcaatggtttttcttgtcactgtgacaagatacctgacagaggtgacttgcgaggtgtctggggactcagtcatggtggcagtggtgaagggtactggtaggaaagtgttaaagtcttgtagcttgggctccaccatccccagggtcctaactgtggcctaggaagacatggtgggtcttcagagacccatttccaggtggtgccctagcctgtgcatccattctggccacaccagagatgactctggcctggggactcactgggctgatctttgagccctcttcctctctagctcacacaggcagccatgcttcatgccaggtgtgaacacagccacgacggggtgagcaaagtctgtgtgctctggtggactttcaaaagcagaacttcgggtttggttttgttttgtttttacattttatttattactggttttttaattttgtgtatctgtgtgtgtgcacatgagtgcaggtgcctgaggaggccagcagagggcgtcagatcacctggagctcctgtaagaggtggctgtgagctgccctacgaggatgctgggactcaaacgttttgttttgttttgttttgttttttgagacagggtttctctgtatagtcctggctgtcctggaactcactttgtagaccaggccggcctcgaactcagaaatccgcctgcctctgcctcccaagtgctgggattaaaggtgtgctgcgccaccacgcccggctgctgggactcgaacttaagtgttgctctaaactactgagccacctctccagcccatttatttgtgtgtgtgtgtgtgtgtgtgtgtgtgtgtgtgtgtgtgtgtgctgataatggaacccagggcctcacacactctaagtaagtggcctgatactgagtcatgccgcagacactaaataggatttcagcctggcagaattatatgagagcagggtgcaggccaggtgtctctttccttcagtccttcatggggttatctccagaaacaaaggcctcttcttacacaatcacaaggatcgaaatcagagaatttaacatcgccctaataagagcctctaagctggtgctgctgcataaaataacaaaacaaactgtgcttataatttcagattaggggccatctttgtggctggtggtggggggagatcaaagcaggaactccaggcagctaatcaatccacttacaggaacagagtcacactgaatgcactcatgcctatagctcagatggctgtctccattcttatacagtccatgagccaaacctagggaatagtgccgcccacagtgggctggctcttcccacatcaattaggggaatccagacaatttcacacagtgagagtctcttcacaggagattctaactatgtcaaattgacatttaaaaacttaccaccagactgtgtgaaaagattttctgagaagagcttttgcttagtgttgggtcttagaaatctgggtgtcggtcaggtgtggcagtgcttgcatttaatcctagcactcaaggcagaggcaagggaatccttgggagtctaaggccagcctggtctacatagctatttccagttctttacatagagagactatagaactacataaggaaggggcagagggagggagggagggggagagagagagagagagagagagagagagagagagagagagagagagagagagagagagagagcgccaatgtcttatcctcttttttttggttcatcccaattggtttcctcttgttttgctttgttgttttgagttgggggggggggtctcactgtgtagcctggcaaaaaagtatcctcctgcctcagcctgcacagtcctaggattgaagccatgagccacagaccgagttctgccctggactctctttgttttgaggattgagcagacatgtgtgtgtgttatggtgttgaccatggacagtgtgggtcacccgggccttgtttcccatcagaagagtgtttgaggccagacagtgggagtgaatggatgaagaggagtcagggtggacaagggagactgggaaagccgaagtctgggctgagggagaggtgggatccggaggttcaggggcaggaagggacatgactaaggccctggcaggtacagtgagacaggaagtggagagggaagctgtggatggcatctgtggatggcatcgcactgcagggtgactgcagggccatggtggttgctcatccctccgcatgcacagaaaaccattgcccaaagccttggtttccacagtcctcatcagctctcaagcccctatcaagagaacatggcacatcgttgtaagtgtaagggaactctgtaaacttcagattatctcaagtgactgacacagctactgcagacatgtgctgtgcatgttttagcttgctttctttctttctttctttcttcctttctttctttctttctttttttttgtgtggtttttcgagacagggtttctctgtgtagcccttgctgtcctggaactcagtctgtagacccaggctagcctggaactcagaaatccgcctgcctctgcctcccaagtgctgggattaaaggcgtgtgccaccactgcccggctgctaccgtactgttatgaatcataatggaaatgtctgcgttttccagtgggcttttgtgggcggttacgacccacaggttgagaacctccggcctagggcatgctggccaaactgtgctaaactgtgctgagttctggtttttaatttgcctcattgggaggatggattagtgagtggggagccaagggagcaggcagaacattgagggaccgctgaagtttcctcagcaacaggtgactagtgtcattgctggtccctgtggtttcttctgcaggggtgccccagcctctgtgctcaaggcacagttctcaccctgcctcccctctgcaggtccatgaagctgacctacaacgaatcaagggtgtttgaaggcattcccacgtatcgcttcacggcccccgatactctgtttgccaacgggtccgtctacccacccaacgaaggcttctgcccatgccgagagtctggcattcagaatgtcagcacctgcaggtttggtgagtgtcctctaagtgttctccacctctaggctgaggtgagagggtggtaccgggattttatctgctgccagcttgctgtcccattaatctctggtctctgcacccatgatgatctcagtgctacagatagggagctaaaggtaactggaaaaaagataacagccaccctgataaatgtcccaccggcacgcgttatccacaagccgtgtcgctccgaagtccgaagggtcctgccccgaggttaagattccatcagtgggcactgcttcttggccaaggagaggagcctgccctcatcctttcctgcttccggaggcaccacattccttggcttgtggtcccttcctttctcaacctagcatccagcttgagactgggtagcgcgttgtggcccaagttagcttcaaacttgcgcatccttcctgcctctgccttcagaggtatcgggatagaaggcatgtacccagacaccagatccccccaaaacactccgttctatgaccacctcctcctcctctatccagcccatagctttcaggaccccagagactgactgggtagaggccactgggataacctggtctgttatcctggtggctctaacttctttgcccactaggacgaggctggaaacattccctgtggccattcacctgctgggagcaaaagctagagtcctgcccttcactcaaactggccacgctcctattgtgtcaaactctcctctttagccaagttgggtgtgaatcttggtactggctgtggccccctggcacatctgctctgtgttctaatcctgggccctaccctgctgctattgctcaagtctgctccgtgcctcacccctcacctggcagtcaggcccaggctgctctaagcacctcccacacagtctttctccttcccacccaccttcttcccgtgtcactcgggtgcccttgctctctcgccagctcgtattgcaccagatgtcaatcaaactcaaatgaaatgcttcctctgatcctgatcagatcctcccagacactctccctgatcttccgtcccctctggctctccagccgctctctggtacactctctcttatgccttcctgttgcctgctcatggctgtcgatgttatttactgaaccctcccctgcttgtctcctccttggactgcaaaccctgtggccatgtcccagtaggatattgggagtcaaacttctaaggcccctacacagacgtgggatccagggcttgcctgcggacaggaagtttcactcacaactgcacctccgccttcccctccttgtaccagcaaaaatcccagggcttggttgaactggtccagctctgacctagtcattaggctgtgttaattggccaggctgggtcccacacaccgcaggggcagcaacaggcccacatcacacagaactctgctgtcaggaacagaatttcagctgtgaaagagaatctaagtgtagttgtgtctgtagcttgttcttttgttttgttttgttttgttttccggagacagtgggtttaagtatgcgtgtgcgtgtcgcacacgcgtggagggaggtataaacaggtgcatcgatggatggagaccggaagtgagtgtcttccttcatcactctttgtttgtttgtttgtttgtttattgatgcagatttttcaatcactaatcctggatcttgggaggtccagggatccacctgtctctgcctcccagttacagatgtggcgctgctgggaacccgaattcgggtcctcacacttgcacaggaggctcttaactgactaaactgttttcccagcccctaaagcttgccccttgtaaatcgggtgagaacaagaaaggggaagaggaacagggattcgagtgatagggaacacggccatgtgaccaacaaccccacaaagaggcaactcttaccacacccaacatctccccttcctgctcctcctgcccactggcctgacttttggtctctcggctctctgcctggcattactgtttacatagatggagtcacaactgttctttctccctcgatccctgatgcctcccatgatggctctcatgattatatggtaaatctgcaagatagcgggtgatagcctagattgttgggtggtgttccatggtggggctaactctgcttgccttactccttcctggctcacggagctttcttttggcatttcaccactacaaggatgcttctagaacattctcgcacctgctgtaggaggagtgcacctgagttcgaatttctgtggcattttatcttaagataaagaatacacataggtttccctttctgggtcacgagactttcttggactcaggactccccgtgtttcctgtccttgtcttgggagcaccttggatcttccctgaagtggtccttaatcaccacgcgggtaatcctgagcagttaagtctggatacacgggccataaagctataaattaattatacttattgcccgggtgcgtctctctgagggtgtttggaaatcaaaagaaaagatgagccacacctattgttagaaagccacgggcatgccacctgcagcaggtctgtgctggcagccttgacatcctgttggaatctgtgcttccaggtctggttaggtgggggcttcatctcccggggatccatggcatccctctgaaagaaatggggttctctctgatctttgctggtgtctcctttcacagtatggttggaaggatgggtagggttttccaagtagaaggcagcatatctcagcgtgactttagaagggtccacagaggtgacgttaggcctcagggattgatcaggtctcttttctgttctgttgtatccttggtgttgtgtttccatgagggcttgaaggccaccctgaggatccttcgcctgtctttctgacaggaccaggtgtgtctctggagcctcgaaggtgctagggttcacctggactctcagtgatccgggttctctgctgcatccctgcccacgtgcacctcctggctaggccatttctctgccaaggctgtcacaggtgcagcactcttgtgtctgacacctgcacttgaagatgcccatagcagccggttcaggggttgggagcaagaggtaggggttgggggcttcaagcagtggatgtcctggagaccagactcggatggaactcagagatccacctgcctctggctcccaattaaagaggtatgctagcctgtctggctttggattttaaagaaatgttttcttttttttaaaaggtttattatttattttatttatgagtacaccatagctgtcttcagacatgccagaagaaggcatcggatccccattacagatggttgtgagccaccatgtgattgctgggaattgaactcaggacctatggaagagcagtcagtgctcttaaccactgagccatctctccagccctgtttgttgtgtttttgagacagagtttctttttgtagtcttagctgtcctggaactcactatgtaggccaggctggcctcaaactcagagatctccttgcctcttcctcctaggtgctgggattcaaggtgtgcaccaccagacccagctaaaacacaatgccccccctgtggatctgttgccctgggagcctcttgtggcctccccagggagactcagaactcggaactccccaggcccttgtcctgagatctctgggttgagccactttggctggttggtggtaaagtcccacctcagggccctgggtgggaatatctcattggtgctgtcccttccattcctgtgatgtcagagacccactcacgtgtacccctgagctacaatgtctccaaaagcagtctgtgtgcctggcttgacattcagactccgcttccactcctaaattcatatggtgttaattttcctttccactttcatctcccatgattccctgctgcacctccagacctggaggggtgggggtggggggttgaaaatcacgtccaagctctcacctcttgctacactcccacccggaatgcctttcccttgccgggtagcagctgctctctcccttcctccacccacccacccacccacccccaacactgtaatcggccactctggctccatccacccttcccaggtcctgtgtgtgctcagcttggtccccagtgttagcatccctgcatccctggcaccgaggcaggccaacgtgacgctcccgtcttctaacgcctctcttcccctcctccaggtgcgcctctgtttctctcccacccccacttttacaacgccgaccctgtgttgtcagaagctgttcttggtctgaaccctaacccaaaggagcattccttgttcctagacatccatccggtaagctccgtgtcctgctagtggctggggcccattcccagagattgccactggccgctgggcacccgccccgctccccccaattccccaggacagagcgggttccaactgaggcaggggagttcagttccttccacagcggctgctcctagaacttcaagtcatcaaagtcttactttaccccaattccagagttcagtgatatcattaccttttttattggggcctataaaaagatataagatagtaaattaattaagacctatcagccgggacgtctccttcaccagagacagatacccttgacagtgtcaagacactaaagacatcatcgtgggccagtgtctcacagagaaatcagggggaggaatttgtcctctgttaacttctctctgcctctggtcactgtaagatgtgtccagtccagggttcctgccctacgtgacgccctgaaggcattccgtgaagaactcttttttgttttactgggtggggatggaagcctgggttttgaacacacaaggccccaggctctgatgccgagctgtgcctgcagctggccttgaacttgcaaatcctcctgcctcggcttccagtgtttcttcacacctggctcatagctgagggttatcgcagaggaagggtgggcacatggggcacagtcgtctgaagtctttcccaggttgtgcctagctccccagcaaggaacttgacatagaggtgttttatatcaggaaagcccctcagaataggggggcctagttgtcgactggggacctgtcctgtgagcctctctccctggggcagatccatgcttcatcccagaggacgaaggtgtttggtattattaactgtctatgcaaatggcacagtgaggccaacacactagcttagagaatggggcaccctcacctcaccccccagaacttaggttccaagaccaacactgggcacctaccctggacgacttctagggagggttgccgggcccacccatggtatgcgtggttatcacatccatgcctctgtccaaaagtcataccaggtttctggaggcctttgccctgggggcaggagggatctgggcgtcctgggcaaaacgtgtcctggccttctactgtgggtgattacaatctgagaggtggcctccggcctcccatcaatctttcaagagtctcatgatctgggcagggctaaaggcatcaaacggtggtctcttttcaccaaccttttccattctagaactttcttcctgatttaaataatacctgtgaattctcctagtgattttttggttgttgttgtttttaattttatgtatgtgagtacactgtcactctcttcagacacaccagaagaggacattggatccaattccagatggttgtaagccaccatgtggtggctgggaattgaactcagcacctctggaagagccgtcagtgctcttaaccgctgagccatctctccagcgagatttttttttaaaagcatcacattttaaaaagagagctgcaagcaggcagctctctgtggctacctctgttagggagcgccaggatgctacatagtgggaaactggctcgattaaacctactcaccctggaccaggcccttcctttggttccctgggctattaggaaatgtaaacaggagaaaaaagacatccttactgcgcatgccccacacttgggaggcactgtcttaggcagcttcgtgagggctgggaggatagcttagtaggtagggtgctcccctgccctgcacagagccctggcttccatcccagcaccatgtaaagttaggcataagccagtcctcagaaggtagagacaggaagatttggagttcaaggtcatccttggctacatagcaaattggagtccaacctggaccacatgaaatctgtccccaaacgaacatgcagatgtggggggcggggggtgtcatccctctggcaacaagtgtctagagaggggatcttgcgatagctgatccctttgggagcaggtatttcctgagggagagcccagtgggtgtgtgggaaatttctagaagggtggatgccccagcaggtattatctggtttaagttggataagaaaccccagggctggagcaggctggagcaggctggagcaggctggagcaggctgggaggctccccacaggatcttacatctcctcactgtatttatgctctaggtcactgggatccccatgaactgttctgtgaagatgcagctgagcctctacatcaaatctgtcaagggcatcgggtgagtggagttgggacgggagcttctgaaagcttcaggggccaggaggtttcatgcattggtgatgcaaggcacaggacagggcgaggcccaagcgttgggatccaaccacctcttttcctgggattgcatcattgctctggatggagactcagacccaggctgcatcctgacactacctccatcctccacgcccaagggctcttctgccattagtcgcattggtaatcttgtctcccccacagtaggatggatttgcctgcttgtatgtctgtgcaccatgtaagtgcaagagccgccgaggccggaagagggcatagggacctggagttagacggagttgtgagccactgaatggatgctgagacttgaacctgggtcctgccagtgttctcaaccacaattccctgggtttactttatgtttagctttgtgtaacgtacagagtgagactgagagtcctcctgcccccattccctgcttgcccggcagaaacatggtggtaggagtccagccttggggctggagagatgtcttagcagttaagagcactgacggctcttccagaggtcctgagttcaattcccagcaaccacatggtggctcacaaccatctctaatgggatctgatgccctcttctggagtgtctgaaaacactaaagtgtacttatgcaaataaaataaataattctttgaacgaaaggacccacatgctggtcactcctcagcatcgcagatcacatgcctgggaaccactgaggaagaggtggactctgggcttcatggtgaaaactaagtccgggaattgctggactcagctaggcttctgtacacacacacaccctgggactcctgcatgccagaaaacactctttcactgaattacatcccagccctcttcactttttaggtctcactagattacccaggctgcccttaatctctctgtagcccatcatggccttgaacatgcgccacttgatctgactcatctgggctcttgaaaccggaggccaaagtgggctggccacgggcttgagttcctgaggtcagcagccaaacattgatttgctccactgcctcaaatactctttagaaatctttatagatttatggagaggtgtcagagacgcaagcaaatctttgttctctgtccccggtgcattcagtactgtccgctcctggcagtatctgtcacggtaaggaaccaaagtctagtgcagcgttattgccacaactgcagcttctcagcacgagatgccgcccgctcacgcctcacgcccggctcccgtggctgtgatgggttgttttgttttgtttttttcttttaatctttgtttcttgttggtgttgtgagttttgggattttgttttggtttttggaggggaaggtggttcatacagaggccaaaagaggatgccatgtgtcctcttttatccctcttccctttcttccctcgcggcaggaattctcaccgaagccccagctagcttctacctgtctcagccctggggtcacaggttcaaagccacacacagcttttagcctgggttcaggggtctcacctcgggttcatttgcttgagcagcaaatgctttccccactgaaccatccccagcacagtgatagtttctcctctagcttttggggtgttaaagtacttgctggacctgggtgtgtgtggtgcgatcaggttcccctgtagatagtaacaggggcagccacccaggctttggtgggaggagactcattattattttattattattggtattattattattattattattattactactactactactactactactactactatttggctttttgagacagggtttctctgtgttaataaccttggctgtcctggaactcactttgtagaccaggttgacctcaaactcacagagatccacctatctctgccttcccaagagctgggattaaaggcattgtgccaccacatacacagatattattatttatttttaaagttgtatttatgtattttatgaatgagtggtctgtctgaatgtacacctgtgagccagaggagggcatcagatgccatgatagatggttgtgagccaccatgtggtggctaggaattgaactcaggaccttttaaccgttgagttatctctctagccccatttttagttgtgtttagtgtgtgtgtgtgtgtgtgtgtgtgtgtgtgtgtgtgtgtgtgtgtataggtgcatgccacaatgaacatgtgaaggttgggacaactttagggagcctgttccaccttctgggtcccggggatggaactcaggtcttcaggcttagcagcaagcactgtcacccactcagctgagccttgccacgccccttaattaattttttgcatcagggtttcctgtagcccaggctagcctcaaacttggctaaggatagtcaactgtacgtagcgttacaggctcccctttatctttctgtctcattttcttgtgaagtggtttcgactgagcgaaggcctttcacatggtaaccagtagctctcccactgacaccccaagccccaaacatagctccattgcgagggtcacatcacccctggtcactcactttgtgtcccatgtttccaggcaaacagggaagatcgagccagtagttctgccgttgctgtggttcgaacaggtgagtctatgaaggtagaagggagtctggggtctctgtatgttagctgaggcttttagttcgctttgatgctaatgttatgttaacttccggtacctaaatttagctatttgtatgtttagcctgcccatatgtatgtatgtatggttatcatgtgagtgcccggtacccgcagaagcccagagagcatcaggtctcctagaactggaattacagatggttgtgagccgtcaagtctgaaacgtgggctctcttgaagagcagccagcgctcttaaccacagagccatttccaggttttgttaatggcccctgagaggggggagtggagatggaattccgcaccaggccaccctgcaggcatcctgatctcgtcgcccttcaggtcctaagactagattctaaccagcgaaagagatgactgttaggcgaggaggcttggggccagatccacctcccattagccagtgcctcatttcatgggcgtgccaacagcaagggcgtctgggaaatgtagttctctttggacttctagccaaagcgcccgaggctctctgttctgatttggggcggggcacggcacagtgggatgggtgccttggagacgcagcgtgcagcccgacctttcttctgcagagcggagcaatgggtggcaagcccctgagcacgttctacacgcagctggtgctgatgccccaggttcttcactacgcgcagtatgtgctgctggggcttggaggcctcctgttgctggtgcccatcatctgccaactgcgcagccaggtaagtaggaggggcggccacgcctcggactcggctcgggtttcagccgacctctgtttcctgcagctagctcttgttctacctcctctctcgcaccctgcagtaacttttctctacaagtcctggaaggcccaggaccctcccaggtcggggctaggctgaggctaggtacccagtcatttggccaacaagtgggtgccacttggtgcccgctctggaaagtgcctgccgaaggtagaggtggggggtcagggctgggctggttagagaagcaaaggaaagccaggttaaaccaaacagagaagatagtttttcccaaggaaaatgctctgggctggagttgggtgtgagatggaagtgggaggagccacgtgtggaaccatgaacttagattttctaagtccagagaaaaaagtgaaagaaattagttataaattttgcttgacccaaatatatcagcatatgaggctgaagttttacatcagccatagagcacttgtttagccagtgtgaggccttgtattcaggcctcagcgctgacaggaaaaaaaaataatcaaaaataatgaaaatatttaaaaaagaagaagaaaagaaaagaaggtctggagagatagctcagtgattatgagcacttactgcttgcagggacccaggttcgattcccagcacctacctggtgttctgcaaccaactgtaacccttgttctgtaaccctagttctgtaaccctagttccaggtgatctcaaggcacacacacagtgcatatacttgcatggagaacactcatacacataaactaatacaggtgcctataatcccagcactcaggaaggaaggaggccaggggagaaggatccggttcaaaatcatccctggttacatgtagggtttgatcaaggccagcctaggatacgtgacatcttgtctccgaaaatcaattaaacttcaaaaaaagaatagtgtgggcgactggagagatggctcagcagttaagagcactggctgctcttccagaggtcctgagttcaagtctcagcaaccacatggtggctcacaaccatctgtaatgggatccgatgcctgcagagcaacagtgtacttgtatacataacataaataaataaatcttaaaagacaagaatagggctggtgagatggctcagtgggtaagagcacccgactgctcttccaaaggtcaggagttcaaatcccagcaaccacatggtggctcacaactatccgtaacgagatctgactccctcttctggagtgtctgaggacagttacagtgtacttacaataaataaataaataaataaataaataaataaataaatctttaaaaaaaaactttaaaaagaaaaagacaagaatagtgtggttcagacaggggtaggggtcacttccataaagaaaggatgactgaggaatgtaacatcagggcatgcagcccctgaaagcaggaagcaaaggggtcatgactagaaccttcgttcccttctggcctcaggagtaccggctcgtgcatgagatcttggggttacgacattggtcaagtcccctggggtgcttgctgatagctgtagttgtgaagcagggaatttgaggacaggactgtgatcagctgtgattcaagagggctgctgtctgcaaacaggggagccactggccacctttgaacactcgtacaagagccactcacagggtcccacctgagatgtcctggggccacaggctgggctttctgtgcgtgcttggtgtgtgtgttgggggggtcttcccacctgctctcctgcagtcagccagctggctgcctttcttctttaaggcttgcttgcttgcttgcttgcttgcttgcttgcttgcttgcttgctttgggaagaacggatccctgtggagtccccagcacccggcttccctttccaaacacaggccccaagtctcttgtcatcaccaaggggtgtctggttgcctctggctctccatgttgactgtagctatgaggatctggaggacatcacggtctgggcttccgggctggtgttcctccctgctgctgcccagtgagaactgagccaggctttttcagtcaaccattggaggagagaccccaaaatagaaggcaggcgtctcgctggggtctgggggcaaggttgtttgtcctgcgggtttcatcagagagaggccaaagaagccttgtcctgacaaagctaggcctcacctgtgtgttcctgtccttgagcatctgggggcgggggggttaactgcttgctggtaaagaacatactcagtcatacgccattcataaatcaagagtatgttaatgtttccctcagattgacaaataccatggacttggagaagaggttaagagagagattgtacccagtaggctccaggccctaggttcaaatccagccccaccagaccagaccagagaaattcaaacactcagcaggccagtcccgctgtggccacctccgccactctcttcctgtcggtgactttctgcttgccggacttccggggcgcttcgcccacagtgcagggtggaacaagactctgcaatcttgttctcttctgtccctttgcttctggggggacccagcgtcaccccagtgacacggcatgccatataaactagcttggttgtcttccaggagaaatgctttttgttttggagtggtagtaaaaagggctcccaggataaggaggccattcaggcctactctgagtccctgatgtcaccagctgccaagggcacggtgctgcaagaagccaagctataggtgcgtaccaggtaaccccccctcttcaccccacctactcatagccagtagacctaccgtctctacctatagcatcttcctggatgttattcacatggagagcagttaccctcctgcctctcaccctcctgcgagatgggaatcttgcctcggtttcttggaccctttcagtcattgactctcatttacaaagtcctgttagaagatgacagttagggctggtgagatggctcagtgggtaagagcacccgactgctcttccaaaggtccggagttcaaatcccagcaaccacatggtggctcacaaccatccgtaacgagatctgacgctctcttctggagtgtctgaagacagctacagagtacttacatataataaataaataaataaatctttaaaaaaaaaaaaagaagatgacagttaggccagttgcttctcagtgtcctatcccctgcaggttgctaaatacaggtccctgggcatcccggaaagccaagagactctcctagcccaaaaggtttgaaaactttttttttttttggacttcaagggcatgtaaaaggacacactgactccagttcagaatagagagactgaaaatgaagacgtttaaacatcagttaagcaccagctacatactcagtcagggcctgaggatagaatttcatggtcaagtacacagggtgtgcgacagactgctcaatacagtctgaatttagatggagtcgtgtgtccgccctgtgctttgctgcgatgggtggtccaggtggtgagccaccgagtcaggtatagctggcgtcctggtttacttagtagccatctcgcaaggatccaggaccgtacttctccaagatctccaggtcctgtagggatcaaggctcaggagagaggctgagccctacagggtgtgtccctgcctgggaagggactggaagagctgtggtccctgcaatttggggttagcagaggtgagtgagtgtctcaatagaagctgctattttagcttgtgagccaacttgggtgtgagcactttcttggagggagctgggggtggggggtggaggcggggcttgggggggcggggaggggaggccacagctcagctcctggaggccagctgactatttgctgcaatgtcatttgcttgagaagcaaataaggcagaactctggtgactgtgtgattgtcctggtgagtgccacctccctggaggaggaagaagctctgcagctcagtgggtccccatatgtccctggagctgagaaagggtcctgccaggaagaggtggggagatggttagtgtctgccagtcatggtgatgtttgggtattgccagagtgcgaggggacaaatgtaggctccgagcagaggctccgcagtccctttagcccagatggagcccataggtacacagccatagccctgtccagaccctaatagaatgaacaagattgggcttacctctttactttcccagcttggtctaatcctcgtttggcttcaagattcccctgggtgccccctagtcctggccatgtgctagggagggcagtgtcctctgaggatcagctacagcacatgggtccttgcatagggacctcaccctcccccccccgaccccccagtgcctggctaagtgaaattcggtggggccccacactctgccatgcacctgggtgtgggggtggtccctagaggctgctgctgattgctatagaatggaaacagacgtgccgctagaccaaagggtattattatatgaataacgtccctctgggtggatgggcatttctgtaaggcaagggggttgggggttggcaggcagggttggctgtccggccagctgctgtgaacacagtagcctttaagaaaatgacctgatcctctgcttctttttggtctgtctctcattctgaattctcctctgtcagacacagtacagggaggagccaggacagaggaggggaaatctgcccacccgccctggctgcccagagccttgtggctttgtgtgtgttttcatgggagagaccaagctacagagttaactcttgctttattctctggcaacaagggcgttctgggccactcacgctggcttcgaacttgtggacatcctcctgcctctgtctcccgagtgcggagattccaagcatgcaccactactgtgccttgtgtccccttccccattttacttctgttttggttttggttttgtgttttgttgttttcgttttttgggttttggttttgggtttttgagacagggtttctctgtgtagccctggctgtcctggaactcactctatagaccaggctggcctcgaactcagaaatctgcctgcctctgcctcccaagtgctgggattaaaggcgtgcgccaccactgtccggccttttacttctgtttttttgtctgtccatttgtctgtctgtctgtgtgtggtgaagccagggccttttgcttagtgtagtccacctctgagcttcaccccagcacttcctgttttacttctgagtgagactatctggaatgctggggtgctgtgtcgggggtggaggagtgggatggggggtgctcgttggctttgatccaaagagaagacgcctccctgcagtttccttccctgccactagggggatcggtatgggggttgcagggcaggggtgcccagtgttcttaaacccggccttccattcattttcctaagttgggaagtcttttcaagtcagagtctcccaacttaccaagccatcctttcctgttctgcaccctttgcccaacatcctactgtccctaaggcccgaggccaccattacagcagtctggtgagagtgtgtctctcttacctctatgaacaagagccagttttaatagctgttcctggcccgtcaggtgacagcattttaaacatgtgggcagacatttaagccattcctagtttctttgtgcggcggtgttatctacccactccggactctgcccccttcactgtaacaggccagtctccctggcctcagttttcccatctctaaagggagaggttggaaccaagagacctacatgactgaggagtccccgtgtctatagagtggcatctctgggttcatggcagacagggctggatgtgtcaggaagtggtttctgtgctggtgggatgtgcggtgggactaggggaggtaggcagggcagggctaggtgacaggcttgtgactcagcttccttttttctccacagggtcctgaagacactataagccccccaaacctgatagcttggtcagaccagccacccagtccctacaccccgcttcttgaggactctctcagcggacagcccaccagtgccatggcctgagcccccagatgtcacacctgtccgcacgcacggcacatggatgcccacgcatgtgcaaaaacaactcagggaccagggacagacctgctgccaagtgagcctgatgggccacaggtgtgctcttctaaatggcctgtgagccaggctgtgggaactctagctgctgtcagcccctcctgtaggagctggccctgcccaggctcctgacttccctcaggaagtctttctgtctttctccatcagtctgaaagccttagttcccacagaggacggatctgtcactcctaggggctgggcatatgtcggcctcttgtgccaaggccaggcaagcagctccaggtcctgaccagtttgcacacacactctggagctgtatctggcgctttttctatcgtctctgctatgtcactgaattaaccactgtacgtggcagaggtggcaggcccctcagggtccttatttttcaggcatggggtcaaagctagaggtatgggccgtctacacccccccgccccccggcatctagtgtacctcaccagagggtattcggaggcccagcatcctgcaaccgaccccttttttctactggaagagaaattttatcatcttttgaaaggaagtcatgactgaagcaataaaccttttcactgattcaacaacactggcttctgtgactgttttctgggcagggctgggtctccagaatccaggccacatcagtaggtgttcccatgactgccagcgagtctcctggtgtgaggccagcaccggccactagccatgtttccacctcaaggctaatgtggtatgtggcttggatgcaccaggacaggctagctctgtcctttctctgtcccgtggaaccttctgggccttccagcagtctgtgtccaagatcagaacatccttgtgaccccaagtgacaagcctgcagcgtctgggggagggctggaagggagggtctaacttttgtcccaagttcaagcaggggttctacctggcatctctgagagtaaaaccatgttttgcccttaagggacccactaggaactgggcaatagattttcactgtgaacattaaaaactacatagccgggctggagaggtggctcagtggttaagagcaccgactgctcttccaaaggtcctgagttcaattcccagcaaccacgcgatgactaacaaccatctaatgggatctgatgccctcttctggtgtgtttctgaagacagctacagtgtactcatataaaatgaataaataattaaaaaaaaaactacatagacactcactcggtggttgtaatggtcttcacccttccctttcttagaaaagaatttgaattattgtgtgtgggtccatgccatggcataagtcagagggtgagctctaagagttggttcttttctcaccatgtgggtcccgggcatcgaactcaggtccccaagtttggcagcaagctcctctagctgcggagccatctcgctggccttgcacttccttaatgagcactgttctctacctgccctggaagcattgaaagtttcctacctcatactataaactgcattataatcgtgagacataaacttctatataaggcaaacatttttagtcttgtaagacggggtctagctttgtaggccaagctgggctggacctcatcatccgcttgccttctgctcctaatgctggggtgacaggcacaccctcaccatagccagatttctttttttccaagacaggatttctctgtgtatccttagctgtcttgga

SEQ ID NO: 6 - DNA, 70000 bp, Mus musculus
  source 1..70000: mol_type = genomic DNA
agtgtcctcagaggccagaagagggtatacggtccctcataactcaagttacagaggattgagagccaccaagtggaggtgaaacagagtccaggagctggatcctcagcaagagcagcctctgcccataactgttgagcctcgtcttcagccctttcatttttccacccaagaaaaatgaaaaatgaaaggcctagggcagccacacagtaaacctcagcagaagaccggagttcagttcccagaatctatacagagccgctcacaaccacctctaactccagctcctgagacctagtgccctcttctaacctgagggcacccacacatccgtgaacacagataaaaagctttaaaatctttaagaataaataaataaatagatagatattttaaaaacaaaactcctaatgacagagcttcttgcctcacctgtttcctctacccccagaggctctgtcacctacctaggcatcaactgggcacagcccagacttatctgaggacataacatgaggggaattgcctcattcaattggcctgtggcacatctgtgggagtagctcaattggtagggtactcaacatcctggccattgtctccagcaccacacagacctggcacaatggagcacatctgcagtcccagtatgggcaggtagtggcaggtgatcaggagttcaaggtcactcttggctacgtcacgagtaccagaccaacatgcactacttctcaaagaatggtagggtggaggggcaggagcactcactggacactcttgccgaggacccaagttcaagtattcctagcatccagatggtagttcacaagcatttgtgcatggagccttatctgtgtatgttcgtgagtgtgtgtgtgtaccatttcctgctgagccatgtctctggccctgacttcatttcctagggtaggaaactgaggcttacacgggaccaggaggggctcacactgacacacctagccctggtcttgtggcttcgattgtttcttctctgggatagtaaacactgccaactttcctttcactgggtgctcgggaaggcaaggcagaagaaaacagtggagtggcccccaaagtgggacaggcagggtacatttcacaggacattttcactctctggtgggaacagcgggacacagaagaagcctcatggactccaagtttcttttcccacactcgttggcagccttgggtttgttatccttgcgacaatgccactgtcctcatgaaacctggtgacgttggctgagcaaatactacctccgatattgactcaaaacgtgtacaggggctcaaagttcaagttcagtggggcctgaggcttggaggaggaggggaaaacactgcctcctgtgattacaacattctttagtatgaagtagagttccctcaaaactaggtctcatagccaagtccccaaacgtcttaagcactttggatagatatctattgagtcctgccaaccggaaaggaggctagagagaatgaccatggagcagccatcctcttggcaaaacagtgggggctgtgaaaattccctgtcacatatattaggtgggatagttagggttgggatgttgtttggccttctacacataagaccttggcttccagctctagtactgaaaaaaaaaccaaaccaaaacaaaacaaaacaaacaaacaaacaaacaaaaaaacgagggctgtggtagctccgtggcagagcacctgactagcatgcatgcaaccctacgattaatcagcttatcttattctttccattgtcctcaaacttgatatgtggccaaggacaatttgaacttcttgctgggatcacaagtgtgcatcattataccctctttatgtggtgtgggggatgggatctaggacttcatgcctgctagacaagcgctgtaatactgagccacatccccagttgtggtttgttttttagattaaaaataatgtgtatgggaatgtatgaacatataaatgcaggtgcccatgatggccagaagggggagcaagatccccaggagctggagttataggtgtttgtgaacattgagacttggatgccgggaaccaaactgtggtcttgggcaagagtagtattcgtacttagttactgagcaatttcctccaactctgtttttgttttgttttgttttgttttgttttggtttggtttggtttggtttggtttggtttggtttggtttggtttggtttttgagacaggctcacgctgtaacccaggctggtctccaactcatatatgatcctcctgcctcagtctcccaagtaatgaaattacagatgtgagccaccacacagggcaaacgtatccatcgtgagcagctggcacagtctatgggattgaaactgagaaagaatgtgaaaaataaacaggcgagcttgagaaaggttattctagccttggtgccagttcctggtatattttcagcgctcagggagtttaggttaatgccccaagcttccccatccccaccccgaggggcgagggggaggttaagctctttattaatagccactccgggagctacagtccttgggaaaagggctatggtgagggtagaggaaggaaaatgaggtccccacaggataaaaccaggtgcaggcggctctggctcattatggtgcctccccaaggcacgaggtgtctgagagttccttctgatgttgtctccgcccctgctcctgtccctcctggccggcagggagggcctgctgtcaaggccgctgagcctcctggacagacagggcacagggccaatccagagggacgcaaagcaggacgccaagaaaagaggaagtgtccttggggaggggaccggttaggactggggttctagggagagaaggaagggcatttaaggtgagtccaacagtccaggacttaaaaggagccttaagtagggggcagcaagaggtgacactagagccatcgaggccgatgaggcccgggggacagcagaaagtgtttccagatagcacctctgcagtgccagccacatgtcacactggtctctgcttccacgctcaggaacaggtgctcagataaggatgcttaagaagatggaaggcgtttgggtgtgtcagaacaatgaaggcaagtgaaagaccccgattctctatgagtcccttcacataaactaaaaatctcaaagttacacccaacaatcgcctgtccttttaaataatagaggtgtagggggagatgagatacagcaacagttaactacagttgggggcgggatcaaggggaactttcattttctctatggaacccttccacggcaaggcagagaaagatggaggcaaacagggctcttccatgaccaggcgtgcttgacctcagagctgcgactcttccttcttcagcgcatttatggagagcttgctacacgcagggcctggagctgcagcctgggagggcgccccatctggcgggtagagtgctttgccatgtctggcccgacttctctacaccctgttcacaactcaccctgcaaccttcgttccccgtgtgctgccagcgatccccctccagaagccactctgtccgaggttccaatcactcccctattgacacctattcgttctcagcgttcaaagagtacttcgattccgttgcttttgggcgcagagacttgaatccaggaccttacacaggcaaggaatgcacggcattgtgagctacccacacaccctaattgttccgcagatgagggttcccccgccccctgacgattttatcccagggagactatgagtgtgttggcctctggcgccaacctgcccccggatgtcaaacatctgttctcaaacttggaacctgagcttacttcttcattctctgatgttagacaagccgttcttcccaacaccaccccaagcgtcccactccccttttttaaaaaaaaaaataatcaaccaaacaaccaaacaaaccaaaagtctggagcaaaatcagcataactcccgcagggttgcagagaagtttacgggcggattagtgtaaaccgcgagtgtaagccaagtatggtgcaaagagctgttttggacatggctttaatgagttggtcccttttgtatatgaagaaactgaggctctcagggtaaggaatgttgtcctaggtctgccgttggcaagctcgggacttagcctcagtttccttactgacctcctccccgcagccccgccccgtccaggccccgccccgtcctgcccgcgctgcagcccagctgggctcagccaatcacccgcggcgcgcgtcggtgagcctggcctcgcccgccccggcccggactccgctcgctcattccgccgccgccgtctgcagactcggtaagacaccgcgagggcagcgctgcggcaccgccctgggctgggcgcgcgggtgtcattcttaagagtgtggagggagacccagactgtgcccgtagggtacgcgtgagccgcgcattggagaagcagagggcactccctcccccgcgcagtgacggatctgggggatcccgggacgggaggaaagatgcttggtccccagactgggcaaaatgcctccactcgggacgcgtgggacctcggctcgtggtgtcccgccccacggtggattcactcaccgcgtgggtttggggtcttcattgctctgtgaaagatgcttatgattaggcgtgactccttggtccctgaccagtaggagtgtcaactccctttctgacccctgctcactccccactccgcgggtggcttggggccagtgtggacgccgagcgttctccaggctctttccacccgctactctgcaggtggattggttctcggggtccgtgagggtcgggctcggcagtaggaaggatgctcgcacaggggacttggcaggtggccggtgccctctcttcgtgcgggtccctgcagtatagccttcgggacacacccttggcgcagagacttaagtgttttgcacccctcaccgcggttttgcccggggttcaagggcaggtgggtgtcagagactgcccaaggtcgcgccgggaccagagcccgggagggtgggggcgtcacgtgacctggcctgagcgaacttggcctgggacagccggacagcacggtctgggcaaccaggctgtgcggtaacagcgggtacccttcgtttttcttcatcttattgtgaatcgggtctggttttcgttgatgtcacagaacacagatgaacgctcaagtttttaaggagttaaaaagttcctagtgtgaaccaggctacatacaatcaagctcagtactctcagctctgggccgactctcgttccgtgcatttaaatagttgtccaaaaacagattttagtcggaaactgctaactttggaagttcgcaaataaaatgaaagtgagtttagaagcctttcggaacgcgtatttagttacagcattttgttgcacaggcgtctggtttctggtcattcctgatattcaagcgtgtttatcactgtcaaaaccacaagccaaatgactaaaacacagctttggccttttatttgtgccagcaagcatacatcaccaccacccccttcctagtcggtcgactcaggactcagtgaggacctaaagcagaggactgtggtgtggggaggacagtgcttgctccggggcagtgcagagtagtaggtaggtctgggcagcgtccgcattcagagtcctgggctagactggttttgctgcagcacccggagctcttttgtcatgtaaattattgcttttcttaactaaagcctcgctagacttaaccttacacttgaattttctcttttatctatttatttacttttaaaaaatgagataggatctcatgtaacacaggctgccctccagcctgctctgtacccaaggttggttttgaacccccgatctccttcctgcacccacccataagtgcggtgattataggcgagcaccaccacgcccggctttgaactccttaaccgtctttggaatcaagctccggttaggttttgtatcaaaagtggccgaagcggcttcctgcacaagacaaaacctttcgagtttgagctagaaagccacctgactgcagatgaggggtggaacgcctcccttccgccgccgctgcgcttggccctgacccagtggtcacgaaaccggcaggcctgattctctcgggagtttggggcgctggccggggtttagccaaccctgattcctcttgcccattttgcccattcataaaagcgctgcaatttttactttattttccccttgggtttcgcagcgaaacccttcgaggcacggtgccggctggctgttttgttgtatgcgacagctgtcctcggacaaagcggatcggtgactccgggatggcagcggcgacgcgtcgctccggaaggcctgcgtgggctcggccggtggaacaggatccgagcgccccggacgcgccagcacgggggcggggcacccgtaacctttcccctacttttctcagccgtcacgtgacccggctgggtaggggaaggggcggggcgggaagccgctgttgttagtgcccttcccttccccagcgccttgaacttgcagtctggatctgcaggcggctagagccacgcgactttcctgtactttgaccgtttgaaggtttttttttttttttggttagaaaatatgtttagtcactcttcagtcactcttcatagatatttaccggtaggcccgagtcttaaagctggtggaagtctgcatttgaactagtttagaataaaatggtagtccaaggtggataattttaaagtgtgtttagcagactggacagtcccctgaaggagggagagtcgctcgaatctgggagttaagggacagcctgggaataggcaaagatccatcctcagctcgggaaagaagaaaaagggaggtggggttttaagccggtggcggtggcgcgcctagtaggcttaaaaaggattactgcacacttgaattaatgccaatttaagctcttagtgcaagaatttttgttgtttggaaaataagcctgctgccaagcctgactaccctaactcgaagggtgaaaatgatcccgacttctgcacaccgtgctatagtgtgcactcaaccgccaccataaataaataagatacatggttttgttttaagtaaaaagcaatacaagcatattgtgcaaaattagaaaggcaaaaatggggtgcgatgcagtcacctacttactcagtagttactgcaaaggccaccactgtgagcagttcattcaggactgtgatggtgtgtacctgtaccatctagctcttcggaggtggacagaggagagccggacttaaggccagctaccaccatccgtgagttggagtcctgtgtgggatacacaagaacttgtctctaaaccaaacctagactgcaaagtttgtttatttttttaatctcttacctaacacaaggagtgagttctccttgttgatttcaaatatctgttgccttttgtactcttcaccttgaaccccgtgcaaatattgaggcggacattgggtccccacaggccctgggctaaggccagctttgctctgttgaggtaacgtggcagcctggttctgagaagtgcccaccctttgccctttttgtgcgagttgttctgaccttcaggaatgtttttcggaagttaatagcagcaggcacactgcccgtttcgcgccccgacacctgggtaactgccactttagaaggttgggaaataggatggctactgaccttgcctctgaggagtgttgcacaccgaccatttaggtagaaatgtaagagaagcttggtgtggtacaaaacacttataatcctagggtttgggggatggtgctgaggcaggattccgtgactccatagtgagaccttgactcaaaaaacaaaattaaccaaaccaaacccagcaaagaccggattaggctgggttgtccatgcctataatcccagtccttagtaggtggagacaaaattagtagttcaagatcagcatcagtgatgtacctggtttagggctagcatggactacataagacctgtctcaaaaacaagacaaaatactgtatgcactggtattaatgcactggtgatatatattggagttatgcactggtgctgtgcactggtgtgaatgcactggtgctgtgcactggtgctgtgcactggtgtgaatgcactggtgctgtgcactggtgtgaatgcactggtgcgaatgcactggtgctgtgcactggagttgtgcactggtgtgaatgcactggtgctgtgcactggtgctgtgcactggtgtgaatgcactggtgctgtgcactggagttgtgcactggtgtgaatgcactggtgctgtgcactggtgctgtgcactggtgtaaatgcactggtgctgtgcactggtgtgaatgcactggtgtgaatgcactggtgctgtgcactggtgctgtgcactggtgtaaatgtactggtgctgtgcactggtgctgtgcactggtgtaaatgcactggtgctgtgcactggtgctgtgcactggtgtgaatgcactggtgctgtgcactggtgtgaatgcactggtgtgaatgcactggtgctgtgcactggtgctgtgcactggtgtgaatgcactggtgtgaatgcactggtgctgtgcactggtgtgaatgcactggtgctgtgcactggtgctgtgcactggtgtgaatgcactggtgctgtgcactggtgtgaatgcactggtgtgaatgcactggtgctgtgcactggtgctgtgcactggtgtgaatgcactggtgtgaatgcactggtgctgtgcactggtgtgaatgcactggtgctgtgcactggagttgtgcactggctgtggcacttaggaaaacaaaggtcatctatactctttctgtctcctcctcctccatgtgtggtccaaggagctgagtctcacttctgtttcttaagtcccactagaatttgtcttcctcagtctccctgaccctatgcaggcctgcaaatcattgctgaaggaaggacaggtttggggcgccctttctccgctggactgaaccagcctactgatattaagggggtgagggccttttggaagtttcactggctacacccctagaaattaatgcccgagagggagggcttagaggaagtggttgggcccctagctcagtgtgcttccatggtacgtggctctgggatgacttgcctgacatctctgtccccatccaggccacatgctctctgcagtgaccacgtccacacccacctctcttcttttagggtttccttggctttgggtaaagccatgaaaagagaattcaacaagcctcgttatcatggcctgaagggatattcgtatgtctacaggacagggacactgagtaactccccacaaccctatcccccttgtagtcctggctagcctgaaactcactatgtagaccaggatgactttgaacttgtgatcctcttgctccacttcagataaaaacttggccctttatgaaactggctggtttattaacatactcacccctagcttccagcagctgctcctggaaatgctacaaggaaggtccagccccggagcccattgcgtcctgcgggcctgtgaaggtattctgaaaagatatgaatgagctgcccatgttagtcagttagaagataggctgaggtaccatatgctcgccctgaagcattgagctgggtgggccttgggggatagagccaggacagacacccactttcttgaaggacaggtgttgactgctcaggctcctggtgggataacagagtgtggccttgtattaaagaacacgttctgtgtttgcacactctagtatccttcgattgatgggagctgaggcagtctgttctgccctgtgctgataaagcataggagagaggctgatgtttgtcagtgagctgcagatctggaagtgaacccagatctgtgttgtagccactctctcacctgctgtcatagagcagggctcttagtggcagccatcacctctatatatgcagacttagactgatatcgaagtgcgggcatgtctgggtgttcatttccttctctgtcaaatggcagctctgatacctgctctcctgtgtcccttgagtcccaggagactccattaaggagtgagacctcagtcttagcattcaggtgacaaagataggctgatctctaggtttgaggctggctttgtcttcccgcagtatgacctatgcaaaatcgtgaactttatataaaaaaaggatttattttattcatcttttttgttttttgttttttgttctttgttttgttttaggatcgctatgtatatagcctttactagcctggaacttgctacatagaccaggctagtcttgaactcacagatatctacctgtctctcgctccagagtgctgggattaaagtgtaccaccacacacagtgctttttacatttttaacttttttatttacgagtatgctatgcatctccgtgtgcgcacctccgatggctgggggcatcagctccccaggggctggagacaggcagttataagctgcagaggcactaggagctgtgctcactcctctgaagagcctcaggcactcttagcctccctctagcctggtccctgatgcctgaaaacaccacacatcccatggaaagtgaatttagaaggttgactttggatcttctcctggggctaatgaggtgaagtgccatcttccatgtgagtctgggtgacttcagcatgacccacaactgtgaggaggaccagaagcccttcacaggacattatgttgttaaggtgtgaaggtgtgacagacaagcagtagattgaatggactccgtgctttttccttttttttttcccactgcatggagactggctatgaggattattgacgaacttgtgcaagcagcagacattcttaagccctaagccatgtctccagccccacctaacagcattttctacctggaggtggcctgtggcatgtgacccctgccccacacatcaaaacatttgtagatacaggttctagacgccagtgtcaggactgcacccgggcgggttttctggtggtcagatctcaggttgctgaggtgacaaacatagacaggatgtttagaacagtccctgaggatttgggaacgtgtatagggacagtggtatgaggagaagttcagttttcttgagctaacagattcatgccacttcctggcagtcaagagttcaggctggcatcctgttctggcaagattggacccatgtgctgtttcagaatggatggggtagactgttagccttgttaaagtgtatttcacgtcttattacaaatttttacatgttttgttgttgttgttgttgttgttttggtattgtgaggcagaggtgtgtgtgtgtgtgtgacctgtggggaggtcagaggacagtttgtgggacttggttctcttcctctcctgtgcgggtctcttgaagattgaactcagttgacttggcagcaagagccttctcctgctgaatcatgttgctggtcctctttgttactttgttttgcagtttttgagacagagtcttagatatgcttggtgagcttgaactcactatgtagttgaggacaatctgatcctcctgcctccccctcttgagtactgggattacaggtctgtgccaccaagcctgctttagtggtgttagagatgcagggtcattcggacttaccccatgctagacaagcactctgtcaactaagccatgtccccagtcgtggctacttcccttagaagtgtgtcctctgctggcttccccatgtgtcacatttctctgattgccatttcttaatgatgtgctgcagtcgcctaaattggattttgctttactttgaacttttctttactaagtagctctagctggcttagaactggagatgtagaccaggctggccccaaatgtacagcagtccctctgcctgctcggattacgggcaagctccaccacacccggcttttatttcattattattgttatcattttaaatggaggcagggtctctgtagcctcagctggctttatggccacagagcctttgcattcctgattctgttgtcttcagctcccaagtgctggagctggaacgattcggcatcctgtctagcatgggtttttacagtgtgtggtaggcgggacttctcttgttattttgggggaatgtagactgaattgaaaatagattaaaaaaaaaaaagaaaatagatggaaatcttacttttaccatccaaaaactaaccctgcttagcttcttttaaaaatcagaccatttaaaatggtatggccagggctggagagatggctcagcggttaagagccgactgctcttccaaaggtcctgagttcaattcctagcaaccacatggtggctcacaaccacctgtaatgggatccaatgccctcttctggtgtgtctgaagacagcaaactatgtactcacatatgtaaaataaataaataaatcttttttaaaaaattaataataaaatagaatgatatggccatagaccagaatttgttttctaacctgatctgaactattgtgttcagccaaacagcacaaatggcttatggtgatatgccatgatgtgggaaagtcacctagggctaaattacataatgcaaatgaagactgttcaggcatgaattccaaatttaacctttcagggtgagttcctgtgacctctccacctatacagtaagttaacaacaacaaattgaaatctggtagccaatgtgacagtttccaaggtggagtctttcaagcagggtctagtgtcttggtctggattcgttcttagattggattaattctcataggtacagtgccttgggcctgctttctctcctccacactcttccactcttgcccacgctccaacccctccagtgccttctcccatcccaggacatagcataaggctctcaccgagaaggagtgatcctgaactctccagtctctggagctatgagacccataactctctttataaagtacccacccttggcaactctgttacagcaactgtaaatctactaagacattccttcaccttcctcataccccaagccagcccacagaaggcacaaggtgcaaaggtctcatttctatttgggtatagcagttgtactcagatttttaagtctcttttttatttttgatgtgtgcgcatttcacatgtgtatgtagcccagaggccacaggcagtgtcatctgcagttactcttcggctttcttttttttttctttgaaaggaaggttacccctaccaagtctttcttgtttattttagatttgaagtgatttacagaacttagatataaggctttgaaacctttctttcatcctagagaagctcacatgtatttgtctttttaaaaatatttttattaggtattttcctcatttacatttccaatgctatcccaaaagtcccccataccctcccccccccactcccctccccacccactccccctttttggccctggcattcccctgtactgtggcatataaagtttgcaagtccaatgggcctctctttccagtgatggctgactaggccatcttttgatacatatgcagctagagacatgagctctggggtactggttagttcataatgttgttccacctatagggttgcagatcactttagctccttgggtactttgtctagctcctccattgggggtcctgtgatccatccaatagtatttttttttctttgaaacaggagtgtgtgtgtgtgtgtgtgtgtgtgtgtgtgtgtaatggccttggctaacgtggacttatttattgaccaggctggcctcgaactcacagagctccacctgcctctggctcttgagtggtatgctcgaccatgcccagcctctctaagttacagccatttctgtttagcggagcagagcgctcccagaagttagatcccagcagcagcatctgtccaggggcaaggcccagggttcatcagcctggtgctgttccttgtgagggatgcctgccatgttcctggatgtcctgcctagaattctgttaaggtgccacttcctgtgggtaaccctttctctcaggatttccagaggttgtttactgctagtcaagatggacagctggtgagggattagagctgcaggcagagcctgtgtctgcctggcaatgaatgatgtgattctggtgggcacctaggtcacactgataagagtcattagccttggtccgagtttgtagtaatcaagttttcctgttggctaccatctatcattttggctacagcagtgacgtaagctggtgtgtgttgcaactgcaggaatcttttgctagcctttgctgtcacagctgctactgcactttggaaggtaaggacatgcccactggagctgagcctggacctgattcctgctttacagttggctagctatgaccttacgcaagcgtttgacatctgagtcttgttttcctcgtcagtgaatggggacaaccggggattcattaattagagttgtagttagaatttaacaagctgatggatgagaaacaaattaataataataataaaaccggcaggtggcccatgcccttaattccacttggtaagcagaaggtggaatctgtgagtctcaggccaggcagggcctccctcatagaccctgtcagaagaagaagagaaaaggcttgggatgaggatacggttcggtgagttatgtgcttgccgtgcaagcatgaggagctgagtacggatcccagggcccacataagaagccgggagcagtggtgcatatctgtaatgctaaccctgggattagagacagagaccggaggaaccctggggctcgctggttagccagcctccccacatcggcggtctctgggctccgagggagaccctttctcaaacaataaggtaaagagtgatggacaccgatttggacacaggcattcatgcacaaaatagggagggcattgtttggtttgtgccaagcagttattaggcaaaccccacaccttcttggccatgaaaacaagcctgctaaccatccatgctagtatacggtgagtcacctgcttgcctgttgtcttgtctaccgacctatctggctccccatctgtgggctgcagggctgtgttgcaatactccctggggcccagaatccatttgcataattcagttactcagaagcctgtcatccctgggtaggtcttattgtttgatttatatggtggacacggttttctcattgcagtttctctggcatgtgccagtttctggcggtgtgctgggcctctcctgaacttcgccctcctttcctagctgactgcctctcaactctgcagccctggggagctacaggaagagcagagaggctgagagggcctggtcgtggggtggcagcttggtggcatggccatcgttagggctcatgggctgacctcagaggtgaaatggttttgatgcaggcggcttgactgtggagaaagcaagcccagtagataagtccatcagccaagctcagaggactggcgcttgacaccaagccatgttattaaaatgaagacagtctcaagtcagcaagaagcctcagcagataaagtgcttgctgcgcaagctttgtgacctgagacaactccctgatcccacatacatgtagaagataagagactccacagagctgtccactgcctctgcacatgctcagtagcgtgtgtccccaccccaccccaccccctccatgcacaaacactagaacaatactaaagacaaattaaaaaaatgaagacagttgtggcagaggtaattgtgctgcagcctgcagggttctcatgatttgatcaagagcattaagctttggggtctttgaaaagctttcagcttattggggggggggtgattagacacggcttggaaagggggaatcctttgccagagagcagaggtgagtgcttgagtgccatcttgtgcatctctgtgctttgggaatgtgaagtcagtagctgtccaaaaccgggtcctgagggggctaactgtgatgaaggtaagaacaggtatgacccagtaggggctaaggaaatggaacagacagtgaagtgcttgccttgcaagcgtggggacccaagaaaggtcaggtgtggcagtgcacagttgcaaccccagctcaggagaggtgaattcaggagaacctctggggctcaccaccagccaaaccagcctcgctgaattagccagggagagaagcttggcccttcaggaacaacacccaggattgatctcaggtacacacacacacacacacacacacacacacacacacacaccaagagcatagtgcaggcagagatacaacatagcaagcaggtgccagttaagcaccagacctaatatgtgtccttagtttttggtggaggtctcttgttctgcattgcagtgaggggcttaggagactaaagtagccctctttgggagttcctccttggcaagtgaggttcgaggctggtatgtaggcagggttgggcctggaagcatggggacacctgcgacaaggaagttggcaggtaggatttgggagtgagcagtcacccaccacactaacactgccacttggggcagagcaagagcacaggtctccacgggagcatctttgtgttctctctgacaccagaaaccaagcctagtcatacacctaacggaggttttaactctaggctttgtgggtgtattcctgtccgtgtctaactctaggccttgtgggtgcatccctggccatgtcctcctgggacctgggctagggtagaggtttctgtcacagaaaccacctgcagttttgccctgagccattgtgaggacacagggcaagacactgtgttgtgttaggaacaatcccagaggggcctttcctgtgtacctggccagacttgcttctgggttgaagctgtccgtgtgtttaccctacagggtgtgtcccatgtaacccttttcctctggttttgatttctaatccaaaggctaaagctaggtgtggtggttatatatactgtaatcccaatatgagggcagccacaaggtcaaggccagtctggtgtagacactgagttgtccttgctgagcagcttgggagggacagtgagtggcagagggcaggtcaaagcccttctctctgtgcccatgtgtctccatcatgcttctgtccagtgtgtgctcttgtggccctctttgaacttgggtcccaggtgcatgccctacaggtatagtccctgtggtcctggtgctgtgatggctcagcccctgggggaatatacaaagcagagcttcatggggcacttgggacatcttgtgagcagtaatagaggtcggggagttgggggggggcactgaaaagccgagtctatacccaggcaaagaagggtgcaggttgaggaggtgctggtgggctaacacacctccttcctctgtgggttctgtgcctgggtgagtgaagcagaggcagggagattattttgggtaactgatggttcctgtgtgccccactgaggcaggccaagtaggggagaggtcaggtcgagagggtgcagcttgagagatgggctgatgtccagtctctgtagtttgggtcttggaaggtgggtttgtaacatctgatcacatgcacaggttgtagaggtctggttagggacaggggccctggctagctcctagggtaaagagagccaaagaagctcctattagtgcagacagagatcggggctggaggtctagcagacaatgtggtgggcctggcctgtgtaatcgaggactggttgtgggaaggcagtgctttagtgccccctgcatggagccaccatggaggtctgccgggccctccctgcaacagcctttggaagggaggaagcagctgcccaatgtcagtggtctgtatccaggcacagctggcttgccagacagaatggctgggacagttggccccgagggcctttcttccaggacgtcaggttcccagcttggtagacacaagaatgcagccttgtgcccaggctgggcagggtgtgatggcaggaagggagagtgggaagggagagtgggagtcagagcagtcagggctcttactctccggcctcaggtgccagctggggaatggattggctttctattggcaagggcacaccaaagaagaatgggggatacggcaagatttgtggaccacatgcacatacatgcttgcatgtacaaatgtgtacactcacacaggtagcccatggctgctcagaagcccagggttccagggaagaggtgtctgaaatgtctgcgtgtgtgtatcatgtgcattgcagtggccttggagcctagaagaagacattgggccctctggagttggagttcaggcagctattgagcctcatagtgttggtgctgggaactgaacttgggtcctctgcatgaacagagtgtgtttttaactacctagccacctctccagcccgaggtgttcttatttatttatttttactttttattgattctttgtgagtttcacactgtgcaccccagtcccactcatcttcctgtcccctcaggtcagcccttgtaaccccaaaataacacacacacaatagaaaaaacatctcatcacataagctgtagtgtgttagtgtgtcccacagtatatccgtctgtccacacatattcccttgcaacaaggagtcattggaagtgtctggcttctgtgacaccatcaatactgggtcctcactgggtctcctccaggttatgctgttgttaccctgtgtcgtggaggttctgcacctttggatcagtagtaccagcttttccatgcatcccagcgattgacagatgatacagattttggggtgggccaattcagtgccctgggtctgggcctgggcagtagctgagctgatggtcagcacactggctcttcctcaccagcaccactagggcaagttctccagcattgtttcagctaagccatccaatgccaccatcagcaggaggcagagctatgccctcaggctggttcacccacacccatgccttcagagccagctccactgtgctgcccagtcaaggtgcagggcccactttccccagggctgcaaccagtaagggggcagggccagttctcccgctctcacactcttggagctggctcccccttgcctttgccatcagagccagctccactgagttgcccaggcaaggtgcagggcccactcttccgagtgctatagccagtgagggtgcagagctaggtctcccactctcatccctagggaccaactaactaccctaactgctgagatggtaagggaaggagggaaggcatcacccccgcatccttgccatctcccagcagacaagtggcagagcctgcctcccccccttgctcttgtcctcagggttggagcacccacacactcttgaccagggccagcccttctgtgttgtccaggtgaggggcaggaccagctcatctgctctcacagccctgtggtcagctctcccaactgccacaggtggcaagagaggagggaagtgcatcacggccaaaaccacatcacctcctggcaacgtggggccagctctcccccacccccacccccatccccaccccactccactcttaccctcaggggaccagctcacctgtgctcccaccaccagggccagctctgctgtgctgtccaggggaggggcagggcctgctctcccgagggctgcagctggtgagggacggggctagctctcgtgagtccactgccagagtcaactctccccactctcacaccatgagggccagctctccagagtgctgtagccaatgaggggcagggccagttctgcacagcccttggtcagccttatggtcccctatggctgtcccaactaggaacttgcccatgtcctctagtggtaatatgagtcaaggatagaaacaccggcccctgccactgggtagccattgactcagacttggcccttaacagcagcaagctgggacctgtaacaagctcaggtggcagggctggccactcacaacaggcgactcctctccaccctcgagtctccagtcccatctcttcataatactcaagctgctgcgcttctccctctcttccctctgaccacccattcctgcgaattgtggtggctcctgctgcaggctggtcatgcagttggtggcccctgggtgacatcctcagtctgtacaacagagcagcagcctgtgctgtgtgccagagggcagtcctgtgggtggcattgtggtctgcaggccttgtcttccttctcgttacactgcgtggtgacaggcggggctctgtgtgtctgtggcctgcctgtgccacggggcagctctggccaccaagccaggcatcaaactaggatgaacaaatgactgccctaaccctataaggttagactgctataaggacagcaaggtgtctcttcgcctttggctgggggttggggggtgctgtattattaattgaagaggaccagattgccataggtctgtgtcaccaacaggccattgctggagcaggtacacaggtgaagttctgtactgacctgtatgtgttgtgttttataactgagcatttgaggaagtgttcagttaaggccaagttcatggaggctttgggggaactcttgcaagctccttggatttgtgggcattttgcttcctccatgggactgtgtataccccagtaccttctccccacacatccagccggctctgccaggtgcaccaggcctccaatcccacgcttgagggtgaactcgcttttggtgctttgcagagagctactcattctaccacagtggcttcaaatggtaggtgagggggaccgagggtgtctgacccgcagcctgtggggcacatgcagctaaggctatgaaaacagcccagcacatgctccatagcattgagtagaggttagaagacagctttcaggggttggttcgcttatcccacccacccgtatattctgggaattgactgtgagtgggcaggacttggcagcatgcacctgtccccggtagcaatcttgctggcccaggatgtttttccttttttctttttcgtaacttgattgattgttgtagataagtaatgttctgttgaaagtcaaaacgtgagacaccctgcctacggttactttccctcgagggcctctgggatgctggctctgagtcagggatgaagagagccttccccacacgggcctgggatcaggtttactaagcagaactgtggtgactgggttgggactctctgggactactgtttctggggtcacagagcctcccacagggacatctgtgcccacctgaaaaggagggagctgcaggtggcagtttctgccaggtgtggggtgtggtctccagggcaggaagagaatggcaggaccagcgtgcatcccgaccagcaggtgtcattgggtctggtcaaagtggagagtggcgtatgtacacacatgaactcaatccagatcaggccacaaaagcccaactgggcaaaacagtgatttttggcggggagaggtatttacgggaatataggggagggattccttactggagcagaaattactcaaagacagctgccttcccaaggcacaccccagcatgggtgacagcccacaaagctgggaacttagagcacactgcacacactgatggcccctcacacattggagagagttctttccaggagcctcggttagtctaaacctctatcagttagcttggctggtttctgcttcttttaggcagctggtctctagaggcttctttgaagcttgtcttgtctttgtcttagtagcagctctgttgttgggctagtctcagagggacccaccgcttttattgctttctctgtcagtgagtgcctagtggatctggtcagtttcagggacttcctggaggtgtttacccttgttaaggagcctgcctgcatgatggggtgttttggaggaaatggatacacaagaagtgggcatgggatgggagttgggagcttgggttgctgcagctgccttcccctccctcctgactccacttgcaggagtctgctgtgctgtacgtgggtggagcaagtgcttacttcacagcaagcatgggcacccagttctgcttagcatccatgagagctctggcttactgaacagacagctgtgtgggtacattcagagctgtggggagacgatagacttgaacagaggaagagctcagaggatggggcttcctgtggtcactgcccacctttccgggctctcactgaagctttgcccactcttcctcacccaagccctaggtgccatggaatccatgctgggattcgtgtgggtcagaggtcagccgtgattgtcattccttgcacagtctgccttgtttttttgaggcagggttgaagcaggctggctggtgagcccaagggatcctcctgtgtctgcctcccagtgttgggattacattgtgcctggctttttttgtagattgagctcagatcttcattccgctcagcaagggacgcgcctaacagtctgagctgtctgcagatgctttggtatctgtcctaccgcaccaggggatccctgaaaaattacttagttctggagggtgatatttgttctgaatgaagccttggtgggaacagtggtggggtggcctgctagagccactccttccacactgatggccgcaactgtgtgaccagagatggatttgatgcttaagtgtaagtgttaagaagacaacatttgtgttttgtgagaggacatttcctttttttcctttctttcttcacagttttttctttgtgaatgtgcagtatattcatgtgtatgcgtgccttcatgtgtgtgcatgtggaggtcagaggtcaattgtcttagcagtggtcagttttctttccacttaaaaattttagtttagcactggggaggcagaggcaggaggatttctgagctcgaggccagcctggtctacagagtgagctccaggacagccagggctatacagagaaaccctgtctcaaaaaacaaaaaacaaacaaacaaacaaaattttagtttactattactgtgtacatgatgtgtgtgagagagagtggggatgcatgcaccataatgtatggagggcagaggacagctctggaagtcagtcctttcctttcaaatttttttccttttaattaaaagaaatttggggggggggtgtgcacgcgcatgtgtgcacatgagtgtaagtgtcctcagagagcaggattaggacccaactcgggctgttttcttcaggtgcttcacaactgaaccacctccctgttctgttttttgtttctttgtttgtttattttttaaagacagtttcttacttctgtaggtttcacagcaaacctcctgcctcaaccactcagagtctgggattgcagatgtgaactaccatgcccagctgagacagggttggttttttttttcccctaagtgatatgtttagcagggcgtggtggtgcccacactttaatcccagtactggggagaaagaggcagaggggtcgctggaagtccaagaccattctggtttacagagtaagttccaggataactagggctacatataaagaccctgactcaaactaccaagacaaaacaaaaaagataagtttatgcatcaatttttaaaatttattttttattgattgattgattggtttttcaagacagggtttctctgtatagccctggctgtcctggaactcactttgtagatcaggctgacctcgaactcagaaatccacctgcctctgcctcttggtgctgggattaaaggcgtgtgccaccatgcccggcaaaatttatttatttttctgtgtttggatagtttgtctgtatgcacagctatgcaccatgtgagtggagtgtctggtggccagggagaccagaggaaggcatcacatcccctgggacaggggtcacagatggttgtgagctaccatatgggtgctgggaatctgacccaggtcacttagaagagcaaccagtggctgctgagccatctaggaagagaagctcaatttaaatttctctttatttatttgtgtatgtaacgcgcacgtgtgtgtgtgtgtgtgtgtgtgtgtatgtgtgtatgtgtgtgtgccacataagtgcagtgcccatagaggccagaagaaggcatcaaatatcctggaactataattacaggtggttgtgagctgcttgataagggttctgggaactgaactcagatcttcgggaagagcagtaagctctcttaatgactgagtctctgagactatatttttaaataacaacctcaatgaggtgtgcctaagctagttttatatgaacttgacacagggtatagtcagtcatctgagagggaggaggaaacctcagttgagaaaatgcctctataagaccaggctataagggattttcttaattagtaattgatgggggagggcccagccccagcctattgtgggtggtaccatccctgggctgtaagaaagcagactgagtaagccatgcagagcaagccagtaaacaacatgcactgtggcctctgcatcagctcctgcctctaggtttggagtgagttcctatcccaacttcctttgatgatgtactgtgatacagacgcttgtgccaaataaaccttttcctccccagcttgttttgttcatgtgtttcattgaagcagccgaatcctaagacggtggagaattcacagaaggaattcacccatctaaagtgtgttttggtgatctttgatttattcacagcaacgtgccactgtgtagcattgtaaccagttagggaacatttttatcaaccctcccaaatactccttcttagcctcatccagcctcccctggtgtgtccctggctgctctggtctttccttctctgtaggtttgttctgttctggacacgtcatgtaaatggagtgagacattggcttccttggcttcctgccatgctggtttggtttgtcttggttttgtgagacagggtctcctgtagctcaggctaacttctagctaaggatgatttgaacatctgatcctcccacctcctctctcaagtgctctgacctcaggagagtgccaccatgctgtgttcatgtgtcgctgggatggaaccctgcaccatgtatgtgtaaggtgaaccctctgctgtgtccccagaccctggtgccatgctatcatctgtgtcactgcgtgtgttggtgctgctgtccttcttaggatagagtcatgttcctggtgtgtggctgcatcagtttacccacgctcttcctctgtggaaggggctggctgtactttcctctcctgctgtaattttgtctgtcatgagcattgtgaacgtgtgtgtgtgtgtgtgtgtgtgtgtgtgtgtaggttttcctgagaacttgttggggttagcttagccaaggagaggttgctgggttccatgggttttagttttgatttgttgttgttgttgttgttgagatgatggtcatttagtgtagcccaggctagcctggaattagctttatgcttagcctttcaggaaaggacaggctgtcttccagaatgacctgaacctgtcacatttctttgacagtggcgtaggtaggggacagggtggggtgggggagagctcctgatctctctcctccctctgacagcacctgtcactcattgtttgcattctagccattcctgtgggtagtatgtgctgtgatttgattgtacttctggaagcagtcagagaatttgaattagtctgtgaagccaacatggaggctgtggccgcctcattagatctgtaggcctgagtttctcaggctcatgttttctggcaccagtgcctgcttaatgacttgaagcagggaggctgtgagggcagagtgcattgttcatgcatgcccacggagcagcaggagtgcctgctatcgggggcctttattaaggcctttattaagtttaggcaggtctttttagtctgttggaggcagggttttatgtagaccaggctggtcttgaattcactgtgtagccaaagataaccttgaagagcctcttgcttctacatcgataatgttaggtttataggcttgtgccgtcacacctggtaggttcgggtggttctgaacattagactcaggtcatgtgacagggctatttcaccctggaaaggacaccataaatattcccaggccccatgattgtcctgagaccaggtttggtttccgggcaaacatggggttcatgactttggatagaattaattgcaacactgtgtttaatccactgctgttaacaacccccttgtgagctggggagatgactcagtgggtaaagtgcaggctgggcttgagggcctgagtttgagcctcagtatccatggaaagtgctgagaacagaagcgcaggtcagtttccagtgctggggttggtggtgggagcaggaggttcctcgggttcacggatcactcagccagtcagcgtgctccaggtcctgtgagagaccttgcctccaaacagtaaggttataagccacagaagaatgcatctcgtgttgaactcaggcctgcaggtatgcacacccacgaacgcatgtgcactcacatacacatacagaaaagtttgcatcgaaatagttccccggtgtttcaaaagttcattaatgtatttattgagttcgtttgtttttgttcttgctacaagatctgtctgtagtccagcctggaattcactgttgagcccacaggctggctttgaacttgaaacaatactcctgcctctgccttataactagtgggattacagagtatgagccattgtgcctggctcacccttgaggttgtataaaagattggccaggtggccagccacagagctctgttgccaagattgatgccaaatgtagacattcaccatggctaggccttgttgccctcttcctgcctgtcacaggatgtagatacacaggaacttcccttgggctctaaattatcttcagtaccaggctgtctccacctcaacctttctgaggtcagaacagcactcaaaggccagtttccaggtgtgccacccacaggcattgtcctcttcctcccacatccacctgtccctagccgccccacctaggtgtcctagatgacactgtgccatttccaacacagcaggcccacaggtgcattgtctgatgtctgctcctgccaccccaggaatggctccgcagagcacaatagttcaaggctggagggtccctttgatcagcttgtccagctcacaggtctcagacctgttcattagggtggcatcttcatcttcactagcaccacactgggccagggctgtctgctctccttttcagagtggcctgctgagctgtgtggtgtcctgcctggccctctttctctaactactatgtaggcattgcttgctctttgcctctcagctctgccacaggagggcagaagggtcgtttctgcgagggtctagttcctcacctcttgagttcacgggacgcagacagcaagtatttgttgaatggacaagtgattgacagttgataccatctcattctgcagcttaagctggtagatcactcaagctggcctcaaacttgtggcaatcatcctcctgaggtgcatgtcaccacacacagctagctttggttgatatctttgagttgtattgatgtgtgtgtgtgtgtgtgtgtgtgtgtgtgtgtgtgtgtgtgtattatttatttacttatttatttataatttcttttgcagtgctggggttaaaacccaggcctcacatacattagcaaatgatccaaccactgagctacatccccaattctgcttgttgatatcatgtagcccaggctggcctctaactttcgatataaccaaggataacactgaactcctgattttcttgcctccatctcctgagtgctggaattacaggcatgtactaccacacacaataataagcatcacaccgagggctcgtgcaagctaaacaggcattcttctatctgagctacagcctagaaccctgactgctgattttctaaaaaggcacatatttaaagttttttttaaaatctgagtgttttgcctacatgcatataaatatgcagcatatataagtcgtacctaggaaagacagaagacagtgtgtgatcctatggaactgaagatacagatggttgtgagccaccatttaaatgctgggaactaaagacaggtcctctgtaagaccagtaagtacttttaaaagctaaaccatctctctagtcctgatacctattggttttgacaggtttctcagactatctgggcctcagtaggaaagttactatctgatgagaacatacccataaatttgattttattattgttctgcatagtttttaccttgtatcttttatatatttgtgtgtatacaaatacatatatgtatacacattttatatgtatatagtatatacaaagtgtgtgtatatatagatacacacacacatacacacactttggacacacactttgtaacaaacagctttggaaaacagagggattgcacactttcagaagaggggatacctcaggcagtgctggggagtgagggaagccaggaaggggtgatacgtctgctgtactgagtgtggagggggttggaagctgttgattctgaagacaacaggagtaaatgaagaagagagggaaagaatgcattgtagatccctcttgttctattttttacctgtattagtgtacactgtgtgtatgtgcatgtgagtgtagtacccacagaggccagaagagggcatcagatctcatggagctggaggtatagatggcagtgagctagctgcttgtcttgggttctgggaattgaactcaggtcctctgcaagagcaggaaacattgttcccagccaagccatctcttcagctccagatcctcttattttggggggttggggatctaacctgggcttcacacatgacaggtattgagatagcacctgtagatttctaagatttactgtattgagttgggcaaagtacagaagaggccttgttttgtgcagggatgacgaaggccccatacatgggtgcttaagaccagtttgactttaggactggcctcagaggatcaggaccagagggtgtctgtgcagtttgtagggtgctagtggagtctttttcatttttcttgttgggttttgttttgttttgttttttaagtttttgttggtttttttgagtcagggtttttctatgtagtcctggctgtcttggaacttccactgtaaaccaggctggcctcaaactcagaaatctgccttcttctgcctcctaagtgctgggattaaaggcatacatcaccacaaccagtgttaaaagcatttttatttattacgtgggggtggggcagggcatgcacaccacagtgttgttgtgaaggtcaaaggacaactttgtggagtcggtcctctccttccacctttcctggattctgggatggagctcacatttgtcagtctttggaggactgagcttgtccagccgagctatctcaccaccccacttggagtctttatagcaccatccaaagctggaggtgcagtggtcctgtctgaaggccagggttcagggccactgggggcgtgtttgtggctactccaggtgtcttcacaaaaactgaaggggatgagctgcaagaagctgccatctctgcagttcaggtcaatgcatggccaccaatgtcctttgccatgctggctgagaatgccagtggaggtgtggctctgtgggataggtagagagggttggtgagcccacaccatcaccccgggtctctaaatggagcaggcctgttgggagctacacagtgaggtgtgtggtgtgaggggcaaaactgtatctattgagcatgagtctgttgggccacagggttcacttcgagagtgtcttctcctgctgcttcctggtgccaggcttctcaaagccatctcttgcccctcctccaggtcaccactcaagatggcagaggctcaccaagctgtggccttccagttcacagtcacccctgatggcatcgatctccgcctgagccatgaagccctcaaacagatctgcctgtcagggctgcactcctggaagaagaagttcatccgattcaaggttagttggttgatcgtctctgcagcacttgcttgcatgcacctccctgggtcccgctgaagaaggaatgtgtcttaggtgggtctctgacagagcagtggtctcagtgctggctaaggcttgtggaggacacagatatataaaatggcctcatcgcttcatagctccttcttattgttgggttttgggctttttgtttgtttgcttctgttttttaagacacagtctctctgcatccctcactctcctggaactcactcagtagaccagactggccttgaactcacagagatccgcttgtttctgcctcccaagtgctgggataacaagcctatgctaccatgccctggaaggcaactggagctcatcagattaatgtaaaacaggccaagcacctgatgccagagcagggggcccacttatggggagtcagtgtccaaataggaacacaggcctgccctctgcccctcagctggattccttgcaccttactgaagaggattattttcaaactttaatgaatttgtgtcttatttgcttaaagaaaagttctcaggtagctcaggctagcctgaaattctccatgtagctgaggatggccttgtatatctggttcttctgcctctgtcctccctggtgctgaaccacagacatgtctgttgggttttattaggtttcttagggtttggttggcatcagggatgccaggggtttgctcaggctaggcagtcatacatgttacatgtgtcacaaaaacgaaaggttctcagaggtcagtgtgatgtgacctgtcatcccagcactttagaggctgaggcaggactgtctagcttggactacaatgagaccctatcttaaaaattgcttttaattatgtagatctctgtgtgtctgtgtatgggtaggtgcctgcagaggccagaggcatgtgatcccctgaatctggcattataggtggtcaccagctacctgacatgggtgttaggaattcaactcgggtctctacaagagcaagtgctcttaactactgagccttctctagccccaagaatgtgtgtcttagttaggtttttactgctgtgaacagacaccatgaccaaggcaagtcttataaaaacaaaaacaaaaaacatttaattggggctggcttacaggttcagaagttcagtccattatcatcaagatgggaacatgacagcatccaggcaggcatggcgcaggcagagttgagagttctacgtcttcatccaaaggctgctagtggaagactgactttcaggcaactaggatgagagtcttaagcccacacccacagtgacacacctattccaaccaggccacacctccagatgttgccactccctggtccgaaaatatacaaaccatcacaatgtgtttttagttgaaaggtttttcttcttggttttgtttgcctgtgtttacaggagtggggggagagagagagtgtgtgagagtgagtgtgtgtgagcacacgagtgtgtttatgcatggaggcccaacctttacagcagatgtcttcttctctagcttcatttattgagatgagtctctcacagaacctgagatcactgattctggctagtctagagtctggctagtctagagtctggctagtctagttagccagcttgcctctggagcactggggttacatacaggtgaaagccattcctgcctggcttttacattgattctagagatccaaatgccaggccttcccccagcttgtgtgctaagtgctttacccactgagccatgtcccagcctagttttacttttttgagacggtgtctgaccctagccaggctcctctgtaactaaaggcagttctcctttcttagcctctcaagtgcttggaggatgtgtgagcccccacaccgtgcttggacagttgttgttattgttgttaatatcaatattattgagttaggatctcactgtgtactcatggctgacccggatctcacggtggagaccagactgccctcaaactcacagagctccatctgcctctgcttcccaagtgctgaggtgaaatgtgtgtatccattaagaatttattatgcagggctggagagatggcttagcggttaagagcactgactgctcttctgaaggtcctgagttcaaatcccagcaaccacatggtggctcacaaccatctataatgagatctgactccctcttctggagtgtcagaagacaactgcagtgttcttacatataataaataaatctcttttttaaaagaatttattatgctgggtgtggtggcacacttctctaatcgcagcattcaaaaaacaaaaccaaaaaaaacaacaaaatagaattaactttttgtaagactcaaacatccaacaaagcagaaaaaacagctctccctccccattaccctcctcctggttcccagagctggcggagtctgggcagtgttgagtcacgcaagccttctctccccaaacctcagttctttagaccagttttttaatcgggtgggcaagattgcagcccagctgatgacctgagtttgacccaaagttccacaaaataggaggagagatctgacctctgacctccacctttgcgccttatggtccttgcatgcacctacgtacaataaataaatgtaacaacaaaaaattaatgtaaactttattttgctgggttattttgcagcaatggtatgggggctcctgtaaacacatcttgactcaggaacagggcaagggtagtttgggagctggggttcctgactggcagatagtggcagtgtaggaatgggacctcacagtgcccaggctgccagtcaccatatggagacctgtatggtaagtcaggcctggctgttttaccaacctggagtccctttcttggagaatcttccaagatgcatttgggctcagaagtcaggggagagaatagactgtgtaaagaaaggtaaggcagttgtgactccaggcagagacacagctactgggctttggtctttggtggcctgcatatatggcaccttctccctgctgcctgatgtcccaactgcctgttgccacagggctgctgacaggtagtgatggtgacctcggggaggatgcagctcacagctcacatgtctttccagaatggcatcatcactggtgtgttccccgcgagtccctccagctggcttatcgtggtggtgggtgtgatatcatccatgcataccaaagtggacccctccctgggcatgattgcaaagatcaatcggaccctagacaccacgtgagtaacccacctccaccctctgcatcttgaagaagatggtggtacctctgttgactgtcctcgctctgaaatgtctgagtacaactatgctatcgctgtttttccgcatgctctttgccctcctgacctgctattgaaagtctgcagacactgccacccttcttctgaagactaggctaagagtggcagaggcacggggacctgccttccaggtgtctgtctcctgaggtcaagatggagacagtgatgacctcagagggcatctctgccccagaagatggctccaagtgtggctgttgccctggagttggtccatccagtgatctggtcctctgaacttactgccggtcattgcttagggacaactcagcccttggccccttcacagtggcttagatgcctgcagggaagtgcgttactccttggctttgagttttagtttattctgggaacttaaaaaagtagatgtcatcctgtctagggacaaatcctggccatcttctggtttttctttttcttttcttttcttttcttttcttttcttttcttttctcttctcttctcttctgttctctttctttctttcttcctttctttctttctttctttctttctttctttctcccttcctttcttccttccttccttccttccttcctttttttttttttgagacagggtctcaccgtatagctccagctgttctggcctagaactcagggatcttatctctgtctgtctctgtctgtctgtctgtctctctctctctctctctctctctctctctctctctctctctctctcctggttgttcagctgtagatactgtatgctaggaaggattgcacaagcaaacagatgagagagtggccccagggaacactcacagctggaggagttggtttctgctgactcagagcaactagcagatttaacccagcagtgtggcatgtttaagacagcttgaataggtgctcgggaatcctggaggtcagtctttaatgggtctgcatgccctgccttctgattgagacacccaggagtgactgagaagccagccagctcctcagcctccagccctcctggcagcattggtcattcctttgtaagcaactgtaggaaacaagataaaaatgcctgtccaggcatcctgccaagggtcctgtctgcctggggcctggaacatcgagctggggctaggttctcagaagcagctggcagagctggtcactaaagactggacctgtgctgtcctgagaggtgaggaaagtcctgtgaacgctcaccctgcttccctgagtgttgagctggctgccaggcccaaggcagctgtgaggccaacagccaagctttgctcttacatgggctgtgtgtctcaaagccggattgcacacagccacgccagctactaaaaccttgcttaatctcagtctttatacgatttgtttctttgtcccttagactctattttcgtaaactgggtcatgtctggctcaaatttagaaaggtcaggtgaaggctcagagacttgcagcctgaggggctgttctggaagcatctttccagtgtgggtggagtgcctgtgtctcctaggctgctttggttttgtttgctgagatctgaaaggaaggccatagaggttatcagcacagctgaaagtgaaattacttttttgtccttattggatttttttttttttttttggttttttgagacagggtctctgtgtagccctggctgtcctggaacttgctatgttgctatgttgaccaaccttgtctcatgaactcatagagatctacttgcctctacatgtgacaccatgcccagcttggattttttctttaaattttattctgtgtgtatgtgtactatacgtgtgcctggtgccttcagaggtcagaagagggcatcagatcccctgagaccagagtttcagatgagtgtgagccactgtgtatctgttcccatcagctgcaggaggaagcatcttccaatgacagttgtgctaggcacccatcctgttcttctgggaggcgaggcctccagagcataggtctctcacatgatgttaatgttttccaatatcagagctctgaaggttagatatgctactactttgatcatttgggacctcttatgttatgttttgcttttagatttatttatttttatgtattatgtgttagtcacatattatgcatatatgtttttaagaattgtctatttattttagtacactatagctgtcctcagacacacctgaagagagcatcagattcctttacagatggttgtgagccaccatgtggttgctgggaattgaactcaggacctctggaagagcagtcagtgctcttaacctctgagccatctctccagcctcatattatgcatatattatgtatgtatgtgtgtatgtgtgtatgtatgtatgtatgtgcctgcaggccagaagagggcaccagatctcattatagatcagccaccatttggttgctgggaattgaacttaagataactggaagagcagccagtgtgcttaacctctgagccagactcgatctcttttatttgttccttgacaattgcatacacgtcaacaggataccttgatttaacccatcccaactctccactcaaacctttttaaaaacccgctgagttcccttagcgccccctgctggaggatccctggtcggccagctggcttggtccacttctcagtgagtcttgtctgcagcagccatgttgtgttcacatctggctgcaccctttccaccccttcttccatgatgtcccctgagccatgaaggggaagggctagtttttatttttgtcactatttttccttttctaaaacaactgaaataacccctcaaatagcagcactttgttttgagttttctttcatagtcggtgactttgatttttaactatttaaaaatcttttaagttttttaaaagatttatttatttttatttatgtggggattttttggctttgttttgttctgtttttgtgtttgagacaaggtcttatgtagaccatgctagtcttgaactcacagaggtctacctgcctctgcctccagcttgctgggatatttgagtttcttataaataaagacagcatttaaggttatatctgaggactggggatataactcaggggttgagcccctgcctagaaagcatgaagcccatagttcacccctgggcacagcaaaacccagcaaatgctagcaaagtatgtttgagagactggcaaagcattataatctgaagaaaacgggccaggatccctttgagcagcagttggcaggtcccacccactgggagcagttgccatcgtgagcagctctctgccccacaggagcctctccaccattcctccagagactggaaaaactgtgtgttgttcaagactgctcaggtcgctggccggcctcttgattcttccttgggcctccagagacaggccactctgctgcacccagccaaggctggcccattttgagaggaatagagactgccagggcgcttggagaactggggtccctcaggcttcttctgcagcctgctcactcctgtcgctgggagggtgcttggtcagcccgctttctggtagctgccattaccaccttccatgctgacgtcttagctccttgtctgctccatcctcacagtggccgcatgtcaagccagacgaagaacatcgtgagtggcgtcctctttggcacagggctctgggtggcgatcatcatgactatgcgctactcgctgaaggtgctgctctcctaccatggctggatgtttgcagagcacggcaaaatgagccgcagcaccagaatctggatggtaaccacccgtcacccaccaggggacccgctccgagtcagagcaggacccttgcctccttagacctttcctcctctgttccttggtgtgtaacctgtccccagcctctggctatgggagagctatggctcatgacacttgcatggatggagccagggctgcttttgctaatggtttctgggagttctgccatctctctgttcacctctgtgtgacctgtggttcaggctggatattgagcataagtaccacccaggaggtctttgtagccttaggggccactttgcaggcagacaccagacggttttctgagttggtcagatcacatgatttaccctaatccgtcagtgttctctatttcttcgtgaccttaaggccaaggcaccatgtttaagagagagagagggttataccatatacatgtgtgtttgttaaattataactggttgtgaaggcttttgttgttgtttagttttggtttttgagatgggggctcatgtcaccctggctggccatttcctgactgtagccaatgatgaccttgagtttctggttctcctgcaccaccatgcctgatgtttatgagatgccagtgaccaaacccagagcttcatgcctggcttacccagatgagccgtctgcccagggaaggacatccaaagccccaacgagctgcttttataagcttagctctctggagtagggggaggaggatgacggatcactcctgggacctccctctttggcactcagaccccagcctttccttcccacacacaccattgtatggctgtcacatatcccccacccacccacccacatattgtcacccctcttggcttctgatttttataaagaaaaggtcattttctgcacccaaggtttgtaaaggatttcagtgaatctggttctggtaacattgcctttcaaatgcttgttactggagctcctcctttgtgcatgggaggagtaggtcagcttggccttgacttgcgggccagtgtgcccccatgcccgcacttgtgaacgtcttcaaggtccctttgctgcccaccacgcactcctggcctccatctttcacaaaccagcttccaccttatatattagttttttaatgtctagagtagcaaaacttaaccaacaggcttaacacagtgtcctctaggatggccctgtggctgctgttgagctgagtcccagcacttcctgagcatcgcaaatgagaatcaccgactacaccttgaacccttagagatgccctagctcctgccagcaggaggcagctgccttccatagctggcctgcaaaatcttcctacaggctgtggggggagctcactctctaggctgcttgacctgatgggcagggacacactgctgcccctgggggtggcttgagggtcaggcacctcagctgatcctactccctatgtgcagagcgggcaccagcctcaaatcaaggaccttccaggaggaggcagacagccctcctgcagaatcgggaaggacagtaggcagccacaccagaagacttctcttttcagctgagctctcggtgggaccggagcctgatggtgtagaccagggatggctctgtcataaaacactcagagagtcaaacgcagttgaaccctggctttggttctttaacacagcctcatctttgaaaacgtaacttgtatctccaggctatggtcaaggtcttctcgggtcgaaagcccatgttgtacagcttccagacgtctctgccgcgcctgcctgtcccagctgtcaaagataccgtgagcagggtaggtattatccacactcctgctgagatgccagtgtccttgggatccatgtttctgcctaattgtgactgtaaattaaagttcttaatctttgttattgctctgcagtctctcctgtagggttattataagaatgttcccctgtctcccaccccacagagtccttgcactggctagacaagtgttctaccagtgagctacatccacagccctttgatatatatatatatattggatacaggtctttctagttagcacacactggccttgaatctttttttgtttgcttgtttggttggggcttttgttttgttttgtttttctcgacagggtttctccatgtagccttgactgtcctggactgactttgtacactgggctgtcctagaactcccagagatcctcctgcctctgcctcctaagcactggtattaaaggcatgcgcaaccacttctggtgaacattttaagatctactcaccttttgctttatatgtgggagtgtttgactttgtgtatgtatgtacaccatatgctcgcagtgcctgaggaggccagaagagggtgttggattccctgggactggggttacagatgactgtgagcctccaagtgaatgttagcgacagagcctgggtcctctgcaagagtagccagtgttcttccctaccatccatccctcagccccatgaccttgaactcctgatctcttgctgtagcctcctgatggtagacgagtttcactatccatggcttgatactgttttctctagagcattattgcatctgcagtgtttaaagtgctgtgtcccacctattggggcatgatcacttttttgtttgtttgtttggttggttggttttgttttgtttttttgagacagggtttctctgtattagccctggctgtcctggaactcactttgtagaccaggctggcctcaaactcagaaatctgcctgcctctgccttccgagtgctgggattaaaggcgtgcaccaccaccacccggctgcatgattgctttttaatggtgttactgagatgcagtttaggactgtatagtgtccttgattaatgtgcagagttcagtgatttgagtttgtatccattattttaaactggaaaatacatacaaagaccacatttattattttttttaaggctgggcatggtggcactagccaccttttaatcttttaatcttagcacccaaaaggcaaaggaaggcagatctcttgtgagttcagaccagccagggctacacagtgaaacactttccaaaaaaaagaaaaaaacagaaggaagaaaatggttaaggagatggctcagtcggtaaaacgtttgcctggcaaacatgagagcctgcattctgcatttgatttgtcagcacccatacatgatagcgacagatttgcagcatacactggtaatcccagtactggggagtggaggcagggggatgcctgtggctcactgcccagtaagcctagcctgactggtgaacttcaggcggcttccagggaggtggatagcctgtgtcaggctgactcctgagtgccaccacacatgggttcatcttttcgctctggctctcgggcacgtgtgcacatacacagaattgttttaactgtttccaagtgtacgcccagcagtgcaactctctgtgctttagggtgtcaaggagttgtgaccaccctggattgtcaggcttttttatagccttgggcagaagccacaaaactccctgccacaggcctgccagcttctgttccactttctgtttctagttctgcctcgagagggagtatggtaggaatgggattatgtaatctgtgacttttgtgaggggttccccctactagcgtttatttttctttgagtcaggatcttaaccgaagctagcctcagacttgtaatcctcctgtctcagcttcccaggcaggatgatttcaagcttcatcactgacttttgttttttgtttttttgtttttttttttttgtttttgcttttgtttttgtttttcaagacgggtttctctgtatagccctggcttcatccctttcttaacaaagctatgcttcttccccttttgtggctgataacgctcctcgctcgtcgtgtgggtagaccagttatctgtccatctgatggtgtgaatgtgggttgtgttgtaagcagggaagctgggaatatctatgcatgttttttatctgggtacttgaccgcctctggttgatagcagagtccatcacatcacagagtaagtagcagaggctggctctgtcatgatcctgatctgctgtaaaatggcagcaccctagttctttccagcttccaatgctggagggcacccatattgttatgacttctgccctcttctcagaactgtaaatcaaaaacaccttggcttcaggtgttggttcctggcttagggatttagtccttattgcccttggcaagttcctttccatctctcatctcccagattggggcacaaacgagactcccagccttggcttgggggtggtgcagccacctacctgccccactcttcttcttaaaacaggatctcacattgcccagactggttttgaacttcctagactccctatatagctgagaacgacctagaacctcaggttcttcagtatctgccacctgagtgctgggtcacaggctggcctattacactcagttttatgcagtgctggggatagaatccagggccttgtgtgtgctaggcaagtgatctgccagcttagccatggagagtccaacctttgttttatcagtttggagttagccagacagttgcagctggctctgacccttcattgtgcaaagctggccctatgtagcctctgttgccctggaattttccatgtagaccaggctggacttgaaccctcattggttcctgctacttcacttcctaaatgctgggattataggtattgtccaccacaccctgctcaaactttgtttcctgaccaatcaggcttggcggcagttctgcttcctggaactcactcagtataggtgggagctttcagaatcctcagaaagagccacacttagccaagcgtggtggcacatgctgtcaccccaacacttgggagataaaggtaggaggaggatcaggagttcaagaccagcctcagttatattgtgagtttgagtccaaaaaagggggctggagagatggctcagcagttactcttccagaggtcctgaattcaatttccagcaaccacatggtggctcacaaccatctgtaatgggatctgatgccctcttttggcatgcaagtgtgcatgaaggtagagcactcatatgcataaatctttaggaaaaaagaagattgcgtgtgagatgttcctgtatgttttacccccaagcctgttcaaggtgtgtgtctgccagtctgtgtgagctgaggatctagaccctaaacatggcctgtgtcatgactgccttgttgagcacctgcctggcctcactgggctgtcagggttcccacccatctcaccacaccaacccaggtcatcactgaagcaatggctaggtgtcttggtacctccatcaggaagccgtgaccttaattatcatccacctgcagccctgctcagccctgcaggggactgtcttgtccggtctatctgcctatgtaaaagcagagaggacagactgacttagggaagcttttctaaccttttctacaccgtcttacttttctgaggcatttgaagtgatgcacggcttggagaagggtgtttaactctgcccgtgttttctctctccagtacctggagtctgtgaggccactgatgaaggagggagacttccaacgcatgacagcactggcccaggattttgctgtcaaccttggacccaaattgcagtggtatttgaagctaaaatcctggtgggccacaaattatgtaagtaattctgcatgtagtgctgtgaaagttcttacttgggtaccagggctcacagagttctagctgtggctgagggcagccatactttcccctgagagctgtgctcagagatgctagcatctcttccctcctgcaagctccttagcagcctttgcaccctgctcagggcagggagctgctttggaccctctagtcccgacctggcataccacaggctgttctctcctgtggtggcccagccaccccacctccctttgtggcatcttctccatccaagagtatcacttctgagtgctctggtcagagttcagatgctgttgacagtggcagctgtctgctgggagccagctaagcgtctgtgtgtggtgttgggggtgggggtagcaagtgggcacactgcaggatatgaagccaagagggggaattcaggtgtcatgaataacttgatggaagggaagggattgaaaataacctatgggagccagacacatgcctataggcaagataggtacatgcctatagtcccagcactcagaaggcagaggcaggagaactgtgggctcaaactaaccagggtcacacagggataccagaccctatctcaagaaaaactcttaaatttaaaaaattatttacttattcatgtatatatttgcctaggtgagtttatatgtattacacaaatgcagatgacatggaaactagagggcattgtatctcctaacagttgcaggtgattgtgtgctgcccaacacaggtgctgggagccaaactagggtcctctgtaagagcagcacacactgagccagctctgtagtccatcaaaaagaagtctcaagagctgggaatgaagctcagttggagtctctgcctctggtgcacaatgccctgggttcagtctccagcaccacacctgttctgtcccacacgcatgtatcccgagggcttgagagatagaggcagaaagatcaaagttcaaggttgtctgtgcttctatagagtttaaggtgagcctgaactgcttgagaccctgctttaaaaataaaaccaggagctggagagaaggctcagcagttaagagcatttgccattcttgctgaagacctgggtttggttcccagcactcacaaggtggctcacaactatctataactccagggccaggggatccaacatccttttctgacttctataggtgccatgcacacacatggtgcacatactttatatgtagacaaaacacacacatgaaagacatcttagtgcacacctttagtcccagcactgggaaggcagagagaggcaggtgggtctctgagttcaaagccagggctacacagagaaaccctgacccctggcccctcaaaaaagaaaaattattttaaaaacacaaataataagtagacaaaaatcaacagagaaagtagacaaaaagaaacaagagaagagccagcagatggtgccagacccaggcactgtgcccagcagggcaggaggccacagcacctgggtcatcaggactgggtctaatcctaagcagctggcccgctgtgcatttagactctcgtgcaagccttttctcaggtgtgcgctccagcacgatgctcaaatcatcacaggttgtagatcagcagcccctgggtggctagggcagcttgctgggtaacattgccatggtcactctggagaatcgagacagcacatgctggcctcaggcttagttctgtggctgagatgtaccaggcatcaggaagatcgatctttggggagatctgggttattttttttttcctgcagtgctggggatgggactgggacccaaggcctggtgaatatcagacaagcattcggcacagcactttggcagttaagggtttttggaaatatggcccccaggctagccttaaagttgctacatagcagtcttgaacttgctataaccttgaattcctgatcctcctgcccatgtgtgcactgctatatagttctatgcctagttttatacattccaacagttgagctaaaacccagcccgactctttttatttatttatttaaacgcaatatgaaactatgctctatagtctagccaggtctccctttaagaccagtgtgtctctcaattccttatggcctgtctgcttgagacaactttgttttggagcctgtcatacactggaattttccagaaagatttgcatacatctgggcatgactgctcagcgtgctgacttgtcgtgtttgtaccataggtgagtgactggtgggaggaatacatctacctgcggggccgagggccgatcatggttaacagcaactactacgccatggtgagttggtcttccattcccactgcagccaagatgggtttgtcctgctgggtggcctgggaaagctcctcttgctcctgcttcttttgtccaagtcttcatgttgctgtgaagagcccacctggggcccatgtgttagacccccctgtgtcttaacgggcccctgccctccacccaacagtgcaaatacattttcattgcaaacaacacttgctatgtaagtgcaggctgaccttgaacctgtggtagaacaacaggaagctggcgttgtaatagagcaataacaaaattagggcttgtggtgtctcatgcctgccaggcaagcactggggaggtggagacagggggattagttcagggtcatccttggctacataggtagttcaaggccctcctagattgtatgagatactctttcaaaaaagtaagtctgagccaggcgtgggggcgcacgcctttaatcccagcactcgggaggcagaggcaggcagatttctgagtttgaggccagcctggtctacagagtgagttccaggacagccagggctacacagaaaaaccctgtctcgaaaaaccaaaaaaaaaaaaaaagtaagtctggcgctagaaggatggtttagcagtttaagagcccttactgctcttccagaggacctgagttcggtttccagtacacacctcaagtggttcagaaccatctgtaactccagttgcaggaaatctgatgccctcttctggccactctgggtacctgcacacacatatacacttaaccccacacaatcacacacatacacatagagagaaaaaacatatccaatttgccagaaaaacaaatggacctaaaacagtaacaaaagtgtccaaagttgtaaagtcaggcatagtgacacatagctttaatcctagcactcagggacctggggtaggagaattgcctgaaagctacataacctgctcccaacaacaacaaaagccaaaagcccacctaaaacaaatacaaaaggcggctgtaacttagcacagctgtcagggagcaccccccccacacacacacacaccttgtatacagaccgccgtgtcaccccatgagccctcttttctgtctcatcctcacaggagatgctctacatcaccccaacccatattcaggcagcgagagctggcaacaccatccacgccatactgctgtatcgtcgcacggtagaccgtgaggaactcaaacctgtatgtcaacttgcctttaaaaatatctcttgggaggctagagaggcggctcgattaataaagtgcttgctttgagaagagtgttcagacttggcccagaggtgcccagctagatgcagcagtgcacggttgtaatcccggcactggggagccagagtcagacaacgctggggcttcccagtcagtgaggcccaggtgcagcgaggggcttgtcgaggcctgggtgttcaatcccgggaatccacatggtgggctgagagaaccagctcctgcaagctgtcctctgacccccacatgtactgatacacgggtaaactcctcaggagaacggtttcttcataacatcaggtggcttaagtgtttatctggaaggaacttcagtagcctggccctgctcggcaagtcccccaggtgctaccctagggatgctgagtgatgtggtcagactgggcagtgtgcacaggaaacaaaccgcattttaatcagttcattgcaaagctttcctctgggattttgcctgttggtttaaaacacatttttactacagattcgtcttctgggatctacaattcccctctgctctgctcagtgggagcgactcttcaatacttcccgcatccctggggaggagacaggtgggtagtgcttactggggtctccgctgggagccagcatttgccattttgctgctcggaaccaggaatagttggggaacgttgggaacagaatggtctacagggtctgtcccaagagcgtctgactctgattgttcctcctcttggaatcgctttgtcttttgtcattgggggtggtttgggaatgggatctcacatgttccaggctggcctaaaataatatgcagctaaggatgacccttttgcctcccagtggattctagacatgtgtcatcatgcccagagtatatatgttgctcaaattgaaactcttggcttcatatatattagacaacccaactgaaccgaagttcagctctggcctgaatttcctttgtccactaggcctgtacaacaggcccgtttccagattttagtgtttcttctgttgagagtttctttcgttgggcattttcctcagacctgctggaatattttcacatgactccttgggcaccttgttctggcccatgcagggtttgctgtaagatacccttgagaaccaatgtgcctgagaggctcgatggaggcccagctgctgctgctggcagcctggcctgggctgggctcatctcatctgcttctaacctaggacatccttggaaaaggatggagggagggagacggatgccagatggtgccatgcaggcctgctggggtcaagtgtacagcattggtcacttttaaacttggtgacaaataacgtgagcaaaaccacttagggaggaaagatttattctacctcctggagccagagcctggagcctattgttgctgggctatggaagggcaaagcatcatgggtacaggacatgtgagggagcagagcatttacctctaacaggaaggacacagctcaagaaaagggactagagtcaaaggtccccttcaggctgacaggtgactcagcgcctgaatgagcctgtctgtaaaccgggtgcttgtacttgatgccaggctctcttgtggtaaaaggagacaacagactcccaggagctgtcctctgccatctacacatgtatggggcatgcttgtcctcacacatatttgagctcacacaagataaataaataaattccatttatttattttatccttgagggagctggttttcttcttgtccaggccaggcagcaagtgtctttgccacctgagccatttcactggctccagcatggtcttcaatagtagtgatcacaaatattaactaaaatctgggtaacactgggtatggtggcatatgcctttaatcctagcattcaggaggctgaaacaggtagatttcagtgagtgtgagcccagtctggtctatactatgtctacatagtgagttacaggccagctaaagcttcttaatgaacccttgccacaaagacaagttatcatttatgaaaatgtagtatgctcggtgaggtcgtacagcgcatgctagctgtcagaggcctctcctgctctgtggtgggctctgattgtttgcgtcttctgagacaggatctcctgaaaccctggctcttctgaattccctagctagaccaagttaactcacagagatccacctgcctctgcctcctcagtgctgggattaaaggtgtgtgccaccacaaccggccttttgctatcaattctctttaagatttttcactgtgtttgtgtgtaggaaggacacagccacactatagcagtcatgtgcaggtcagatgacagtgccgtgaggctgcagatcaaacgagggccattggcttacacggcaagggttacccacaagtcatctcccaggtcctcttccccccctttgagacagggtcttatgtagcccagactagcctctaatttgttatgtagaagctggctttgagctcctgatccttccaagtggtacaccattgtgcctggctaaaacattcctttttttttaagatttatttttgctttttatttttatgtatgtgttgcctctttatgtgtatatgcactgagcatatgaagtgcccacagaagccagaagagggcaccagatccccggagctggagatccgggaggctgtaaaccacctgatgtgggtactgggacctgaactctgcctgtgacttatgggagagatctcattcaatactacgtgtgccaaccttgtttcatagataggaaactgacttaggttataggggctggttaagccaaagggagtgactcagtatggagtcaagggccacacagcagtattcccagaactcaggaggtggaggcagaagaatcgggagttcaaggtcacccttggctacatagacagtttaagaccagcctgggatacatgagatcctcacgaagcaaaccggtcaggtcagatgagcccaaggctttctgacttcttgtcactccttcgtctcctcaccacctcagacaccatccaacacgtcaaggacagcaggcacattgtcgtgtaccacagaggccgttacttcaaggtctggctctaccatgacgggaggctgctgaggccccgtgagctggagcagcagatgcagcagatcctggatgacacctcagagccgcagcccggggaagccaagcttgccgccctcactgccgcagacaggtgcgtgtgtgcgtgcgtgcgtgcatggcctcgcacacccttcccggtgtgtttgagggcctcagtccaatgccttaatcctggggtcctccgttcctccacctcctataagcttggatcaaagtcctgtatcacggcattcagttaacttccttcgtttctttttctgaggtaagtgctccaccactgagcagccagggagactctgtgtgtgtgtgtgtgtgtgtgtaatgtagtgtatgtgcatgtgtgtcaatgctgggtgtcttcatctgttcctctccatcttatattatgcagacaaggtcactcccaaagcctggagctcactgactggcttgtctggcagaccagtgagttctagggataatcttgtctctgccttctcagtactaggataataggtcagggccccagatcctcatgcttatgcagagagtactttacccacactccccaacacacaccaaaggagcatgcgtatctttaagaccagcattgtggggttggactgtgggaaagagaaactgccctgtgccctgtgccctgtgccctgtgccctgtgccctgctttgaacagggtaagaaacaggtagagtgagttggtggggagaggattttgctctctggggccaaaagtatacagcacttcacaaaagaccatctttcacagaaccctgagctgctctaagctctaactctttaataaagagttgctccaggcactcaggagataacaaggctgtgtgttgtttggagcctgggactgagaaggaaaaccagaaagcgaggaaggccatagtctatgcttcatctcctgccttgagggctggggtgcactgggggcctgcggcatttgtgggtgttgataccaaaagttgttgagagaggtgggtagtggatgggaggcagcacagaaggcctgaggctgagaactgtgggagggacccaggatccataggaggaagctacttcagggtagctctgaagagagaccaaggtgagcatcatggtgggcacatggaggtcattgtgggagtgtcatgtgaccagtcctggtctggggtcaggggccagatggtgtcgttccttccctgccccaaagtagttgtggcatgatctctgtgcctcaggtttcctcatctgacaaatgggtctgtggtgctgaagacatcagtggactgattgatgcctgagagaggctcaggtcagagcctggctccagggccaggtccttcggcatttgttataacttctgctggggatgtctggggcagagaagggtgcttctccctggctgctgcagagacaggttggatttcattctgagcagtgtgtgtgtgtgtgtgtgtgtgtgtgtgtgtgtgaatatgttgttcctcgggtactagctgtcgacactgatgttggagacagggtccctcactagcctggagcatatccatttaggtgacctgtctggctaacaagccctggggatcctgattttgtctctcagacctgagattaggagcacataccaccacacctggctcctctacatgggctgtgggggtgaaattcaggttctcgggctgacaagcaagcactattgcctactgagccatctccccaccctggacaactgattttagatgcgtttactccttgccacagagtgccctgggcgaagtgtcggcagacctattttgcacgaggaaaaaataagcaatctctggatgcggtagaaaaggcagcattcttcgtgacgttggacgaatcggaacagggatatagagaggaggaccctgaggcatctattgacagctatgccaaatctctgctgcatggtagatgtttcgacaggtaactcccatttttaatctctgaggttgatggggggttgtgggagatgtttctgggggtacctgtgagcagcctccccaaatctgacccctgctatggggttgtactaggaaggaataagcatccaaccagaaagctctgctttcttccctccacacaaactcagacacagttgtaggcccggtagttttgaggtcccctcctttgcttacccaccacccttcttaactgtgggttacctggctcccagcagaactgtgggaggggcccaggatacatggaggaagcacataggacagtcctgggaaccatcactggccttgtgcagttgctgtgaccctgccctcctggatcccctttctgtaggtgtaaagtgaggaggtacctcccaggcctggctggagagggcatggccccagactccttgttacagtagtctacacgcctctggacccagggctcacacacatgctatattgtatctctgatacaagaagctatgaatggctctgtgttctctgtgcccccttgagtcaccttcctaggatgtagaagcctggagaattgtcagggtggagagaagcaacggtctctgcagaaagaagcacccatgtagcttttgggttcccatgtcgatataaccctggaagcccatgatcatgttgtctccctgtaggtggtttgacaagtccatcacctttgttgtcttcaaaaacagcaagataggcataaacgcagagcattcctgggcggacgcgcccatcgtgggccatctgtgggaggtgagccacaccattgttacctgtctgaatgtggaaggtgctttgggaaagaaacccaacgcaggcccacttgaactctgcagtatttgtctaattctgtattggttacatttctctctccctgatgaaataccttggagagggaggagtgagtgtgtgtgagagtgagtatgagtgtgtgtgtgtggggggggtgctagggatggaacccagggcctgcctcatacttgctgggcagctgctccacaccaaggctgtataaccttagcctttattttggagacaaggtctcactagatagaaacatgctagacttgaactcactgtgttgcccagactggcctcagacctggaatcctcctgcctcagcgtgttgagtatcagagattacaggtatgtgccaccgggcctgggttagcatcttggcttataaaatggcagtgtcctggagccagagagatgctagtagggtagagtctgacgatctgagttcaacccctggcacccatgtgacagtagaagggcagcaccccatgtgcaccctcacttcacacaccccatgtgcaccctcacctcacacaccccatgtgcaccctcacctcacacaccccatgtgcatcctcacctcacacaccccatgtgcaccctcacctcacacaccccatatgcaccctcacacaccccatgtgcaccctcacacacctcatgtgcaccctcacctcacacaccccatgtgcaccctcacacacctcatgtgcaccctcacacaccccatgtgcaccctcacctcacacaccccatgtgcaccctcacctcacacaccccatgtgcatcctcacctcacacaccccatgtgcaccctcacacaccccatgtgcaccctcacacacctcatgtgcaccctcacacaccccatgtgcatcctcacctcacacaccccatgtgcaccctcacctcacacaccccatgtgcaccctcacacacctcatgtgcaccctcacacaccccatgtgcaccctcacctcacacaccccatatgcaccctcacacatcccctatgcaccctcacacacctcatgtgcaccctcacctcacacaccccatttgcaccctcacacatcccctatgcaccctcacacacctcatatgcaccctcacctcacacaccccatgtgcaccctcatacatcctaatagttaaatcaaaaaaaattaaggcaatgtatcttactgaaaatatttcagttactaaaatgaccaggttacttaaagaccaccaaatctctgtccccctttctcctaacccagctttcaaaaacacagagttgggtgtggtgacacatgtcaccctagtacaggagcctgaggcaggaggatcaggaacttgaggccagcttggactataaaacgaagtcatttcaaaacagagagaggcaaagcaaaagaaagggtcagacccagactgcagatctggtgcctttgtggacttctcagtcaaatagctgtattgcaggaatctgaaatttattaaatattgacgtaactctcaacgaagttttaaatttttgtaagattttggacttcaggctagggatgctctctctctttccctcccttcctctctcttccagggtctcaccatttagcccaagccaaatggcactgctcttgcctcagcctcgggagtgctgctggtttcgggttgcccggccacccccagctaagactttgcatcttagtttttccttgcagtgctgtgccagcgatttcttttcctgtggtgcagagtcaactgcagggtgcagattgtgtctgcttgtttgtgcgcatgctctggcctccctccctccctcgatcactcaccctctcatcccttgggtgctcggacctctgcttggggtcttggcagagtcccagctgctcggcttcagccagggaagagttcctacacgagacagcctgggaaaggaagaagcaggcagtctggatctaagacaccagcgtgctccgctgagacaccaattccaatggcgaagtgcaagggctcgtgtccctgcgaggcagcccctgaactgatgccagcagcagcctcagtccagatgcagatacctcacctccattagctcccagaccctgcggctcacacacagatctctgggaagctctgcaaagcccaacagtataggcccatgagaatatcccagggacttttcagataacacatgcacagctgccgttgggtaatgagggtctctgaggcccaggcaacttgcagcatacacagcaccgttctcaagggcagacatagagggcacatagtggcacatgcctataattccagcactccagaggctgagatgggagaattactgaaaagcctgaggacaatcagggctacataataaaaaacaaaaccaaacaaaaggttgaaaagagatgaagaagaaatttcagcaagatatccacaaggtctggctgatgttaaggtttcttgtgtgtctacatgtatgtgctcatgagtgcagtacatgcagcaaccaaaggggggcgatagatcccttggagctggggttaaaggtagttgtgagctgggacccaaaccccaaacttctgctagagcagcaggcacttttaacagctgagttggctctccagccctcgggctaatattttaaagtaactgatcaagataaggctaaggaggccaagatcaagataaggctaaggccaaggccacaagtagaatcagtatttagtttcctgtctgcctggtggaatgaggaggactcttgaatggagcctaggttcccaggatagtgctgggcacccagctctcatcttggagggacagtgccccagtaacaatgggctgattctgagatgtgaaaccaggtggggacaacttcgtagtagcatcctgtgtgctgtgggtccagcatcctgagaactgaggatcctcccatcagccctgattcaaggtttctccgcttaccttctcttgacagattcatctcctagagcagctctggctcggggagacgcctatgcttctctcttattacaaagggcgcagaaggctgtggcaggtgggctccagagatgcagaggttcagagccacggtgttaccctccctggtttctagaaaccacccatcagttagctttctggggccttcctgctctttgccctctggtgtgtgtgtgtgtgtgtgtgtgtgtgtgtgtgtgtgagagagagagagagagagagagagagagagagtgtgacagagactggagaaggtgcttggtgtgtggagattccgcccttactcccaggattggtcccaggagtgtggagtgtctgtcctgagcgcagtaggaaggaaacactgctcactcgtggcttctactccatctctgcttctttgtctcagtatgtcatggccaccgacgtcttccagctgggctactcagaggatggacactgtaaaggagacaagaaccccaacatccccaaacccaccaggctacagtgggacattccaggagaagtaagtctggtgtcccagagaccatcactcacctgggtggcaatcttgttcctgagctctggtgataggcaagctctctaccaaacagaaagctgtttgcccagagttggggaaggggcctgggatgctgagagaaatgggacttccgttctcagttgagatttcagctgaaaagtagctgggcttctgtaaattccctgtaaacagggataaaaacttaaatcctagtcaggcaatggtggtacactcccgtaatcccagcactcaggaggcagtagcaggcagatctttttgagttccaggccagcctggtctacaaagctagttccaggacaataaggactacatagaaaaacctagtcttagagattttggggctgtgtaaagatggcctgtttctgtcatcccaacccctgtggctgaggccagagaattctgtgaccgtgtctcaacaaccaacaacagattctgagattcagtccactcaagtaggagccctgagaagagatgttagggttggaaccataactccagcctgtctgatccaagtgtgagtttctggggcctgagaagtactttttcgtgtggtaaagtccagcactctgaggacccaggacagtgtgtgatccatagaggaacaatacctctgtggaggcctgaccttggggccgggctgactggaccaccagggctcactggctctcttcaacacacctgtaatagagccatgctgggttcatggcagaattttgagaaagatgcaggtatttcctggaaacccacggtctgccccctcagcatcctcactagtggtctgtcaggcctcctggcacatcccaggtttacacagcgtcgcctggatgggattgggtgtgtagtgtcacgtgactagtgcccagagtcagatggcagtgacattgctataaacaccatggcctccgcctctcatccagctccttcctggcccctggccaccgtggatccctgtgtttgtcctttaaaaaacaatattatttgtgcttctagaattgaacacatgtaaacaatgtgtcttgattatatccatggcttcctcctccagccagctcccctaaggccctagactagagactgaagtgcagacttccagactgttccgccctgcttccttctcctcctgctgtgcttccccactaagcagagctgccttcttcctctgcagtgccaggaggtcatagagacatccctaagcagtgccagttttttggcaaatgatgtggacctgcattccttcccatttgacacctttggcaaaggcttgatcaagaagtgccggacgagtcccgatgccttcatccagctggcactgcagctcgcacattacaaggtaagaagggcactcactgtgggcactctggagctgtttccctagtgggcagaggctataaagcccagcatggagggcagctgacaccaaacaccagccctggggctacctgcggtaccaaggtgtgaaatgctagactgcagagcagatcacccttggttttgtgacatccctctcaggctcctatcacagatgagagcttgggacataagcaaatgaacagtgtaacctggactggagagatggcctggcggttcagaccacgtgctacctctgcagaggaccccattcggctcttggcaccttcacatagcggctcacagccacctgtaactgcagttccatgggatctggtgccactcttctgccttctgtgaactacacatagttacacatatgcacaaaggcaagcacacacataaaatgttaaaatataaatcttgtaaaacaaccccataataagctctgactcttacagtattgttaagaaatacttaggacaaattgcttaatgtgtctgacttttgacaggcctttccttaaagcagaagttatgagctcctcattgctctaggctgagtcagatttctgtatccacccaggactgtgtgccaattctatcgtttccctgagcagtgctaaagggtggcagaagcaatctgtacctgtgtgaggatgtggaaggaactttagttcttggtagaggtcagcctactccagacagaagcattcagttgctggcaatgcagaagtcattgtggcaccagatctagaagtgcacagtgggagggtggacatcccaggggctcagcttcaggtgccaccagctgtgtccaccacccttgcctccccaggagctcttcctgccccacccctaccctgtttctcttcttgttctctctggcacaactcccaaggcctctgtaatcacttctaaccccagcttcctcgactctctccctagccacgccccctgaccactcccctgctgccttttccccagccctagccacactctcctccagccaggagcctctcatctcttcctgaagctcatcgccagcttctactctgcttccagaaaggcagccttacctgcagacacctttgcttcaagctaaggagtgctggctgcagtagccttccacctgtgatacctcccatcctgccttccacaggtggcccttctgtcccatccagacaataggcttttcccatctgcatctgctcgctcctcctggctactgtcttgtccatggccatagtgactgaactcctcctcattccctagtcctgccctcttgcctgtccctcccctgtgtctctcagacctcagaccttgctgtgtgccttgtcctttctcctaaaaattcacatcagctggagggctttctggaggacattgtggccttagtcatgtcttggaccagaaaccatgcaaagacaaactccaggaggctggagagacggctcagcagttaagagcaccgtctgcacttccaggggatttgagttcaatttctagcactcacatggcagctcacagtcatctgtaactccagtcctcagggatccagtgccctcttctggcctctggacaccaggtttatccttgttggtcagatgtatgtgccggcaacacacacacacacacacacacacacacacacagtgggggaagcagactgccacctcctctcagatccattccaggtcccttctcccttagacttagatggccactcccacctgttagcctgttagccctaagctagttttccttccttcccaggacatgggcaagttctgcctcacgtatgaggcttccatgactcggctcttccgagaggggaggacagagactgtacgctcctgcactacggagtcctgcaactttgtgctggccatgatggaccccacaacaacggtaagaccatggtggcagaacatggcatggtgcatggtttctggacgacactagaacagtggttctcaacttgtgggtcgtgaccccattggggtcacatatcagatatcctacacatcagatacttacactgtaattcataaagtagcaaaattacagttatgaagtagcaatgaaataattgtattcattatgaggaaccatgttacacaacatgaggaactgtgttaaagggagacagcaagaggaaggttgaaagtcactgccctggaagctgaccatgaagctccatagcccctccagctccacccttcctccctcaacccctcctccccctgctcctccacccttaccctacttctgcatcccctcccccagctccttatgcataggaacagaaagcatttaagaggacatacagaagagagtattgctcgctgagctttgggaatgagattatatgtatcgagacccttcacatgcgactggacatggcacagacacaggaaaccggacacccatccctggggtcagagcctgtctgaccaatctaccagatactacagagtactacagtatgttctgcctcttaggagccctgcagtggccttgggaaaatgaactcagatgaatccaatgcattgctggtcccagctggcacatggccacattcagagctatgcctaggttccccaagccctgagacaggcgtttaccaccaacagacaacagactgcattttttctttttttttttttttactctagagagaggaaaaaaaaaaaagcaagcggctacttctgtcactagaacttccaggaggctcctatgaggctgggtctgtcataggaaggccagtgccttgacatacctccatgtagaggccacgtgtctcaggtggtgttgtaatgtccagaagattccaaagcagcctatatgtctgtgtttgttatgtgtgaacacatgtgtatgtatgtacttgtgttatgttgtacatgtgtatacacatgtgtagtgaggccggaaatgtgcgctgttgtcttcatccattacgctccaccctgtactttgagccagggtctctccccgaatgtagagctgctacttaggctagactggctgctcagcaagcctcccccttgggtcataaggaagccctcacattttagcagagtagccaaggagggttagcgttcatccctgtgacaaaataaacttggggtcagagctgggatagagctccaggtttggcaagaactcttaacagcttgagtggtccacatttggctctctgtgtacatagcacacacaccgtcacagggtgcctctccatcaacctacctactgcccagcagcccttctgaggcaagaatctggaaagggatcgtgggctggtggggctctgtcaagtatattggtcacagcaccctgccttttacagaccactgaagagaaagtggcccgacgcacaacctgaaccatgcaaagcatgtccttctcctgttgacccttaggcagagcagaggttcaagctgttcaagatagcttgtgaaaagcaccagcacctgtaccgcctcgccatgacgggcgctggcatcgaccgccacctcttctgcctctatgtggtgtccaagtatctggcagtcgactcacctttcctgaaggaggtaagtgcttggactgtgtgtgtccagcctggcttttctgtgttgagacaggatcttactctgtagcctaggctagccttaaatagcaatcctcctgcttcagcctcctgaatgctgggactacaggcatgcatcagcatactggcttttaacagtggctgcatagaatgagctagaaaagcccttgagaacctagaggtgggtttgtagaggctaccttagaccccacgggctgtcctctgccttccctgtgagaggcagtttggtcaaggggaggggctcagatactttaaccttagtgtggccctgaggggcttggagaaatagtctctgaccacgcagtttttccttgcctccctcttaggggatcctcagacaaaaaggaggtattctgcattgctctctgcattgctctctgcattgctctctgcattgctctctgcattgctctctgcattgctctctgcattgctctctgcattgctctctgcattgctctctgcattgctctctgcattgctctctgctgctgtataaagcactctgaccacttgccccttggaagtcagggcaggaacttaagcaggaacagagtcagaagctgtggaggaaggcttcttgccggcctgtgcacgggctcacatttggttaactttctttacaaatataattttacatgtgtgctttgccttcatgtgtaggtgtatgtttggtgtgtgtgcagtgcctagagggatgtctaggaactagagttacaggtccactatgtgggtgctagcaatcaaagctgggtgctcttagccactgagccctctctctagtcctctccagctagatggacacctgtccagggacagaacccacctaccctgagctggaccttcctgcagcaagacaatttcccacagacatgtctgtcttaatgagtgttttgggcagaaagtgtttattcagcttacatgtccatatcatagtccattactgaaggaagtcaggatagaaacccaagcagaggtggacctgtgaggtgggacctggcgcataggccatggaggggtgctgcttgctggcttgctgcccctggttcactcagcctttcctatagaactcagcatcaccagcccagggatgatcccatccagaatgggctgggcctccctaaattgatggataattgagaaaatgccttacatctggatctcactgaggcagttcctcaactgaagttccttcctctatgatgactctagcttgtgtcaacctgacacataaaacaagccaggacagtgtccacaaatcagtgtgatggcagatagtcactcagttgagaatgtcttgtggttcctcccaggtgattgtcagcagtgtcaggttgacagtcggtgctcactaggagaagcgagtagggaagtcatgtggacattttttgctttgtcgcaggtactgtctgagccatggaggttgtccacgagccagactcctcagcagcaggtggaactgtttgactttgagaaataccctgactatgtgtcctgtggcgggggctttgggccggtaagtgcagccaggcccacccactaacttcaagcccatcccattgtccccaggctggcctcaaactcactcactgtaaagtccaggctggccatcaccatcctcctgcctcagcctcttcagtgcccagacttacattgagaacaggaagtcgccgtccacgttatctcatttcctagtgataaaactgccctttctctctctgcaggttgctgatgacggctatggtgtttcctacattattgtgggagagaatttcatccacttccatatttcttccaagttctctagccctgagacagtgagtatattgggcactactttttctagacatgaggtaaatttctttttggagggagcacatctttaaccaaaacattcatttcaggtgtcatcctttttttagcttagtttcacatcgatgaatttcacccatttgaaagacatatatttatttcatttacctgcctttgatggtagcagtggtcttaagtctcaatgtgtctgtgtttctgtcattctttctactctaaacaactccgtgagcaggttctggccttccgtagggagtaagtctctgagggagcctcttgggaacctctaatggtggccctccgcagtatcagcctagccagtgctacaaaatcttccaggctgcccactcccacccacagaccccgagtcctcaggaagctaggcatgagttacacacctctaatcctagcattatttaggaagctgaggcaggaactgagagttcagggatagcctggggtgtagtacagagtgacagggaggcctcagatctgacccatccatgcatatatcaagccacctatggctgtcccccactgagtgctcagggtgcttttattgctcctccactaattccctcagagcttggagagtgggaagacaggaggttggcctaacccagggacagatgataatgggcaattccaaaagtgagacttgctgcgtgccagagcttttccttctctgtgtctctgtctctgtctctctctgtctctctctctctctctctccctctctctctctctctctctctctctctctctctctctgtgtgtgtgtgtgtgtgtgtgtgtgtgtgtgtgtgtgcttgcacaagcactttatccactgagccatctccccaactcctctcatttggttcttttttttttttttccattttttattaggtatttagctcatttggttcttttttttccattttttattaggtatttagctcatttacatttccaatgctataccaaaagtcccccatacccacccacccccactcccctacccacccactccccctttttggccctggcgttcccctgtactggggcatataaagtttgcgtgtcctatgggcctctctttccagtgatggccgactaggccatcttttgatacatatgcagctagagtcaagagctccggggtactggttagttcataatgttgttccacctatagggttgcagatccctttagctccttggctactttctctagctcctccattgggagccctatgatccatccattagctgactgtgagcatccacttctgtgtttgctaggccccggcatagtctcacaagagacagctacatctggatcctttcgataatatcttgctagtgtatgcaatggtgtcagcgtttggatgctgattatggggtggatccctggatatgggtctctacatggtccatcctttcatctcagctccaaactttgtctctgtaactccttccctgggtgttttgttcccaattctaaggaggggcatagtgtccacacttcagtcttcattcttcttgagtttcatgtgtttagcaaattgtatcttatatcttgggtattctcatttggttcttgaagacagagtcttgctatatagcccaggctgggtttgaacttgctgcagttgatgctcctgcctcagttttcaggcgctgggatttatttgcccggctgctgatgtgctgttcggcttctttcttttaggactcacaccgctttgggaagcacttgagacaagccatgatggacattatcaccttgtttggcctcaccgccaattccaaaaagtaactgtcggagccgcacggaaggaaaatggactctagtgatacaaaccaaatgaataggtgttgctcctgaccataggacaggcagaaaattgttcttataaaactcagttttccttccagaaggtttaccgtcggtctccctagaacaaccgtaggctccaccgtttgacttgtgaccctactacatccagagatgccttggctccaggaatattgggcacagtcccccgaagtcttttgaatcggctcctaatggataaagggatttaaatgctggtgaatccctggattttgggggttgtatcaatatgtgttggaggtgacagacttcctcagtggtgaccctgtggatacttgggacttgacttcacccaggcagtgagagcatcaccttgtggaaagagaaagtggcttcagagccagtggaggtaacagctctagctaacacacctgtaacacactaatggaatggttaggcctggggattaaggttctgctatgaatgacagccaccatcgctttgggagtccacatttgactccacatttcctggaagcaggataccacctcctcagtgccaccttcgaaacccagtgccttaacgatgggagcccattggcaagtggggccatagagaaggcttagcatgtgaagcctttgggtggatatgtgagggtgctgcttcccctcacaagttcctgcatagagatgtccctaagtaagcacttcccccaccctagaagatgaggtccctggtggagggagcgatgcagaatctcattggccaccagttccattaagcaacaaaataacagatgtgtccacagagggaagtgaggggcttggtagtcaaaggctaccaagttggacaccagctggagagtgtggcagccattggcaaggagagtgagaccctggtcactgagtcaggcatactgacacaggcagccaaagccttgtcatggcagccaggagatagagatcctggcagatacaccaacgggctcatcttctaatcccacccagtcagattccaaccagagcaaattcgatagaaggctaggtcattttggtgacagactcgggggtctcaagtaatgggtgctttgtacccaaataccatccctctgtgagagtgcctttcttgacaacatccaatagactgtaaagcaactccgtttggtattccatgtaaacatagcataatggagtggcctcccctacctgttaccatcctgtcctgacaagtttagctcttcgtgttttaaatcatgtatttattttccagtgcccctttggccttgcttgattcctactcgtgtgctagctgtaacagaagtgagggtggggtggccagaagtacagagaggtgctggctgaacagctcatgcgtgttttataagtatccatgaatacaaaaaaaagaatcacacctacaagggccaaagtttttttcctactaaaaacaagaaaacaaaaggcaacataaatatatagcagagacaactgtaagtcaaagccgcctgaccgcgctcttaggactacttgctaacctctgttactcggagtattcctgctagtacccaagtgtgacattcctctctcaggtctccagtgtccttccttgctgctcctgagcagttaccaatgcaattttttactccttccaaggcagaagagtgggctttcactgtaagtgttcaaaggaggaggtaagactactatgtatttaatgtggaacaaaacatagtcttaccgcagccaaggttcgaatttggttttctaatctgtccattgcatgtaaataccatatgctgtttggatataaatcttagaaatgcatgtgtgaacgaatatagctgaccattaataaaacattaatcccgcctactacatcatctgtccctgtctcccttgtcatactaatcagggttatactatcaggtactggctgtgagcctgcagtgtgcagggcaccactgtcatgtacttgaccaaggagcagcacacagtggagctatcctccattcctgtgtgggtgcctggcaccacagaggtgctatagtgcatgttcagtgaatgataacacttcctttttagaaacatgtaatgtgtgtgcctgtgtggcatatgcacattggcatacggctgcaggtacatgaagaggccagaggagttgtccggcattcttcaccccctttctttgagacagggtctgtatctaaacctggagcttatatttttttggcaatgccagtggccagcaagccccaggagtgctgactcggtcccacttcagtgatgggttttaggcatgtgtacaagcacacctaccatgttaagggatgaactctggtatctgaactctggtcctcatgtcatctgtgtcttaactgctgagccatctctccagaccagcactgagtttcttcatcaggctcttccaagcatctgctctaggcacggagaataaagaactagctccctgcctctagttctggatgttagctccagttccaagacagccaactgaaggaaggatcctaagtattgcaactcgagtgaggtctgggcacgatgtcatggacctcgaatcccagtactaggaaggtgaaggtaggaagatcatgactatgaggccaacctaggctacatagagagttccagactaacctgatctatatagtgaaaccctgtctcatagaatgaaacaacatgaaactggacatagcaattaagaacacttgttgctcttccagaggacctggggtcaatgcccagcactcatggtaacttgcaaccacctgtaactccagttcagagcatctgacaccctcttctgacctccaacacaggcatgcacatggtgcacatacagacagacaaaacatccattcacataaaatttttttttttttttggtttttcgagacagggtttctctgtatagtcctggctgtcctggaactcactctgtagaccaagctggcctctaattcagaaatctgcctgcttctgcctcccgagtgctgggattaaaggggtgcgccaccaccacctggcaaaaataaatctttaaaaaatgtaaaaataaacaagagccccaagagccagatgtaggaagtggaagctggcacaaggaagtgcccagctgggcatgggttagaactgtgaccaacaagaataacaagacagaggcagctttagccaacctactgggacagcagcagtcatggaggtgccaggcttcattctgaccagcttggtgacataccagtgtctgtgtttatctggtggataggtgtctgagactccttaccattctttcttggcaatttttgattgaatgcactaggattctcaggctagcccttaatgcagtgtgcttattacagctgtggaacatgtgtgcacagacccaggagagcatagggagatacgagccattgtgacagtctggtgcacgacaagatgtcagcctcaccttccatttaaacagttggtgagagactttcatagtatgtaactgagtcggggctcgcctcattttaactggaagaagccgccagagcactgtatagaatccttctcaacgatatgggaatgcaaggtgcaaaggaattcttgctgccgactgtgggatgtggattagcttacagagtgcttgcctggcatgcaagaagccctgggtttaattcccatactaaataagctgatgatagtggtgcatacctattatcctagcactcagtaggcagaggcaggaaaatgagaagttcaagattgcctttgattacatagtaaattcaaggaagtctgggccacatgagaccttacctttaaaagatataagtaaaacatgagttggaagccaacctggactaaatagtgatcaccaggtcaactagagtcacgcaacaagaccctgtctcaaacagactaacaagaaactaaaatggctgatgaggtagcttagtgggtaaaggtgtgtgctgccaagtctgacaacctgagtttgatcccagaagccgcacagggaaggagagacaactctcgactctggaacgttttcctttccttcagattaatgccatggtgtgtccacaaatgtatatgcaaatacgcattaagtgtaaataaacaaaaccctgaaagcaaaacctgatggcacaggattttaattgcaactactgagactgagacaaggtaacacgttcaagggcagagttcccagcatgtaggaggccctagattcaatctccagtactgcctgagcaataatagataaatgtactggaagtgactcacaggttaatagcactttttttcgctgaggacccaaactcacttcccagcacctattaactcagtttcgtaatgccccccccccccccaggttggaccgttagggaagggggcggggccatctctgcctggacagggcgggccttaacaggggtgggtggggcctcccacctagcactgcttcaaatttcctggagtattagtcatgtttttaaccccaatttttgggaggcagaggcaggtggagctctgtgagttccaggccagcttggtctacgtaaagagttctaagccagccaagtctacctactaagattctgacttaaaaacaaaaacagatgaaaaggaggggtgaaaatgatgtaaatacagtactcaccaatgaaattttcaaagtttaaaaaaagagagaaagaaccaccacctgggagcctcagtttcccccactaggtgcaggagaggttggctgatacccagtccctccagcctccatccaagcataccgaaaggctgccttgtgggccaaccacagcccaggtcccatccatcatgctcaggccccgcccaccaagagccaaagcccaacctccatggcaaggccccgcccaccatagacacccacctaccacaacccaggctcctgtcatcttggcagaggccccacccaccgcagcccaggctccgccccgttcagtcagagacgggccccgcccctttccctaacggtcaacggtcgtccagcctctcagaagcaaggcgagctggacggccgcgtcgtgtcgctgttctcgggtcccagtggccatggaggacgcgctgctcggcgccatgactggccccgaagacgagctgggc

SEQ ID NO: 7-8 - skipped (2 entries)

SEQ ID NO: 9 - DNA, 20 bp, synthetic construct
  source 1..20: mol_type = other DNA
ccagagccaacgtcaagcat

SEQ ID NO: 10 - DNA, 20 bp, synthetic construct
  source 1..20: mol_type = other DNA
cagccgtgcaacaatctgaa

SEQ ID NO: 11 - DNA, 29 bp, synthetic construct
  source 1..29: mol_type = other DNA
tgaaaatcctcaacactccaaactgtgcc

SEQ ID NO: 12 - DNA, 20 bp, synthetic construct
  source 1..20: mol_type = other DNA
gccctcagaaagctctggaa

SEQ ID NO: 13 - DNA, 21 bp, synthetic construct
  source 1..21: mol_type = other DNA
tagggtcgaggctctgcttgt

SEQ ID NO: 14 - DNA, 30 bp, synthetic construct
  source 1..30: mol_type = other DNA
ccatcggtgcaaacctacagaagcagtatg

SEQ ID NO: 15 - DNA, 23 bp, synthetic construct
  source 1..23: mol_type = other DNA
tgatggtgttgaggaagcttttt

SEQ ID NO: 16 - DNA, 21 bp, synthetic construct
  source 1..21: mol_type = other DNA
tccctcaagtctcctgttcca

SEQ ID NO: 17 - DNA, 26 bp, synthetic construct
  source 1..26: mol_type = other DNA
acaacagatcgcgtgatgaccgtctc

SEQ ID NO: 18 - DNA, 16 bp, synthetic construct
  source 1..16: mol_type = other DNA
gcatgttctcacatta

SEQ ID NO: 19 - DNA, 16 bp, synthetic construct
  source 1..16: mol_type = other DNA
  modified_base 4: mod_base = OTHER; note = uracil
gcatgttctcacatta

SEQ ID NO: 20 - DNA, 16 bp, synthetic construct
  source 1..16: mol_type = other DNA
  modified_base 6: mod_base = OTHER; note = uracil
gcatgttctcacatta

SEQ ID NO: 21 - DNA, 16 bp, synthetic construct
  source 1..16: mol_type = other DNA
  modified_base 7: mod_base = OTHER; note = uracil
gcatgttctcacatta

SEQ ID NO: 22 - DNA, 16 bp, synthetic construct
  source 1..16: mol_type = other DNA
gtctgtgcatctctcc

SEQ ID NO: 23 - DNA, 16 bp, synthetic construct
  source 1..16: mol_type = other DNA
  modified_base 6: mod_base = OTHER; note = uracil
gtctgtgcatctctcc

SEQ ID NO: 24 - DNA, 16 bp, synthetic construct
  source 1..16: mol_type = other DNA
atcatggctgcagctt

SEQ ID NO: 25 - DNA, 16 bp, synthetic construct
  source 1..16: mol_type = other DNA
  modified_base 5: mod_base = OTHER; note = uracil
atcatggctgcagctt

SEQ ID NO: 26 - DNA, 16 bp, synthetic construct
  source 1..16: mol_type = other DNA
tgaggtcctgcactgg

SEQ ID NO: 27 - DNA, 16 bp, synthetic construct
  source 1..16: mol_type = other DNA
  modified_base 6: mod_base = OTHER; note = uracil
tgaggtcctgcactgg

SEQ ID NO: 28 - DNA, 16 bp, synthetic construct
  source 1..16: mol_type = other DNA
ttcagtcatgacttcc

SEQ ID NO: 29 - DNA, 16 bp, synthetic construct
  source 1..16: mol_type = other DNA
  modified_base 6: mod_base = OTHER; note = uracil
ttcagtcatgacttcc

SEQ ID NO: 30 - DNA, 16 bp, synthetic construct
  source 1..16: mol_type = other DNA
  modified_base 5: mod_base = i
gcattttctcacatta

SEQ ID NO: 31 - DNA, 16 bp, synthetic construct
  source 1..16: mol_type = other DNA
gcatattctcacatta

SEQ ID NO: 32 - DNA, 16 bp, synthetic construct
  source 1..16: mol_type = other DNA
gcatgatctcacatta

SEQ ID NO: 33 - DNA, 16 bp, synthetic construct
  source 1..16: mol_type = other DNA
  modified_base 5: mod_base = p
gcattttctcacatta

SEQ ID NO: 34 - DNA, 16 bp, synthetic construct
  source 1..16: mol_type = other DNA
  modified_base 6: mod_base = p
gcatgttctcacatta

SEQ ID NO: 35 - DNA, 16 bp, synthetic construct
  source 1..16: mol_type = other DNA
  modified_base 4: mod_base = p
gcatgttctcacatta

SEQ ID NO: 36 - DNA, 16 bp, synthetic construct
  source 1..16: mol_type = other DNA
  modified_base 5: mod_base = p
gcattttctcacatta

SEQ ID NO: 37 - DNA, 16 bp, synthetic construct
  source 1..16: mol_type = other DNA
  modified_base 6: mod_base = p
gcatgttctcacatta

SEQ ID NO: 38 - DNA, 16 bp, synthetic construct
  source 1..16: mol_type = other DNA
  modified_base 7: mod_base = p
gcatgttctcacatta

SEQ ID NO: 39 - DNA, 16 bp, synthetic construct
  source 1..16: mol_type = other DNA
  modified_base 9: mod_base = OTHER; note = uracil
gcatgttctcacatta

SEQ ID NO: 40 - DNA, 16 bp, synthetic construct
  source 1..16: mol_type = other DNA
  modified_base 5: mod_base = i
gtctttgcatctctcc

SEQ ID NO: 41 - DNA, 16 bp, synthetic construct
  source 1..16: mol_type = other DNA
gaggatggcaagcaca

SEQ ID NO: 42 - DNA, 16 bp, synthetic construct
  source 1..16: mol_type = other DNA
cacctgcgggaagctc

SEQ ID NO: 43 - DNA, 16 bp, synthetic construct
  source 1..16: mol_type = other DNA
tgtgccccagcccatt

SEQ ID NO: 44 - DNA, 16 bp, synthetic construct
  source 1..16: mol_type = other DNA
cttccacagtatatct

SEQ ID NO: 45 - DNA, 16 bp, synthetic construct
  source 1..16: mol_type = other DNA
tactggtagtgttgct

SEQ ID NO: 46 - DNA, 16 bp, synthetic construct
  source 1..16: mol_type = other DNA
ttgacacaaagggagt

SEQ ID NO: 47 - DNA, 16 bp, synthetic construct
  source 1..16: mol_type = other DNA
gaatctccttttccag

SEQ ID NO: 48 - DNA, 16 bp, synthetic construct
  source 1..16: mol_type = other DNA
tttacacgcttccgcc

SEQ ID NO: 49 - DNA, 16 bp, synthetic construct
  source 1..16: mol_type = other DNA
agactctcggttccga

SEQ ID NO: 50 - skipped

SEQ ID NO: 51 - DNA, 16 bp, synthetic construct
  source 1..16: mol_type = other DNA
cttttctatcagtctc

SEQ ID NO: 52 - DNA, 16 bp, synthetic construct
  source 1..16: mol_type = other DNA
cttcttgatgtctttc

SEQ ID NO: 53 - DNA, 16 bp, synthetic construct
  source 1..16: mol_type = other DNA
aagtgtcactaaaacc

SEQ ID NO: 54 - DNA, 16 bp, synthetic construct
  source 1..16: mol_type = other DNA
ggactgaaatagcaga

SEQ ID NO: 55 - DNA, 16 bp, synthetic construct
  source 1..16: mol_type = other DNA
aggctggcccccactg

SEQ ID NO: 56 - DNA, 16 bp, synthetic construct
  source 1..16: mol_type = other DNA
ggtttttgattcttcc

SEQ ID NO: 57 - DNA, 16 bp, synthetic construct
  source 1..16: mol_type = other DNA
gcatgccgccccgtcc

SEQ ID NO: 58 - DNA, 16 bp, synthetic construct
  source 1..16: mol_type = other DNA
tcttcatacaatagca

SEQ ID NO: 59 - DNA, 16 bp, synthetic construct
  source 1..16: mol_type = other DNA
cgttcaaattccgtct

SEQ ID NO: 60 - DNA, 16 bp, synthetic construct
  source 1..16: mol_type = other DNA
tccggctgcggctcag

SEQ ID NO: 61 - DNA, 18 bp, synthetic construct
  source 1..18: mol_type = other DNA
agaggagaccgagcgaat

SEQ ID NO: 62 - DNA, 23 bp, synthetic construct
  source 1..23: mol_type = other DNA
catggtttgtgttgatgtacgac

SEQ ID NO: 63 - DNA, 24 bp, synthetic construct
  source 1..24: mol_type = other DNA
cctacatcagggagcgagaaggga

SEQ ID NO: 64 - DNA, 21 bp, synthetic construct
  source 1..21: mol_type = other DNA
gtcaagactacaacacacagc

SEQ ID NO: 65 - DNA, 23 bp, synthetic construct
  source 1..23: mol_type = other DNA
aaaactataaggaggggtgaagg

SEQ ID NO: 66 - DNA, 33 bp, synthetic construct
  source 1..33: mol_type = other DNA
ctgaaggacttttaaatgtagcctgctcactaa

SEQ ID NO: 67 - DNA, 20 bp, synthetic construct
  source 1..20: mol_type = other DNA
tgcagggagccactctgagt

SEQ ID NO: 68 - DNA, 24 bp, synthetic construct
  source 1..24: mol_type = other DNA
agaacccccactgacttatctgaa

SEQ ID NO: 69 - DNA, 26 bp, synthetic construct
  source 1..26: mol_type = other DNA
cacagagcctaagatgtgcacgcctg

SEQ ID NO: 70 - DNA, 20 bp, synthetic construct
  source 1..20: mol_type = other DNA
ccggagctagaagcgatcaa

SEQ ID NO: 71 - DNA, 24 bp, synthetic construct
  source 1..24: mol_type = other DNA
cctttagcttctcagcctcttcct

SEQ ID NO: 72 - DNA, 17 bp, synthetic construct
  source 1..17: mol_type = other DNA
ctcgagtcagggagatg

SEQ ID NO: 73 - DNA, 20 bp, synthetic construct
  source 1..20: mol_type = other DNA
aatgtgcctgctgtccttga

SEQ ID NO: 74 - DNA, 20 bp, synthetic construct
  source 1..20: mol_type = other DNA
  modified_base 5: mod_base = OTHER; note = uracil
aatgtgcctgctgtccttga

SEQ ID NO: 75 - DNA, 21 bp, synthetic construct
  source 1..21: mol_type = other DNA
agatcaatcggaccctagaca

SEQ ID NO: 76 - DNA, 18 bp, synthetic construct
  source 1..18: mol_type = other DNA
cagcaccttcagcgagta

SEQ ID NO: 77 - DNA, 24 bp, synthetic construct
  source 1..24: mol_type = other DNA
aagaggacgccactcacgatgttc

SEQ ID NO: 78 - DNA, 20 bp, synthetic construct
  source 1..20: mol_type = other DNA
acatgacaggcgcgatctct

SEQ ID NO: 79 - DNA, 25 bp, synthetic construct
  source 1..25: mol_type = other DNA
tctaggttcacgtacacatctttgc

SEQ ID NO: 80 - DNA, 26 bp, synthetic construct
  source 1..26: mol_type = other DNA
ttccttcaagcaatgccctcagcaat

SEQ ID NO: 81 - DNA, 16 bp, synthetic construct
  source 1..16: mol_type = other DNA
gttattgtggttggcg

SEQ ID NO: 82 - DNA, 16 bp, synthetic construct
  source 1..16: mol_type = other DNA
attctgtgtgcactgc

SEQ ID NO: 83 - DNA, 16 bp, synthetic construct
  source 1..16: mol_type = other DNA
tcttgtctgacattct

SEQ ID NO: 84 - DNA, 16 bp, synthetic construct
  source 1..16: mol_type = other DNA
ttttgtgtcttctgta

SEQ ID NO: 85 - DNA, 16 bp, synthetic construct
  source 1..16: mol_type = other DNA
ctgtttgagttttctc

SEQ ID NO: 86 - DNA, 16 bp, synthetic construct
  source 1..16: mol_type = other DNA
caaagtgataccagtt

SEQ ID NO: 87 - DNA, 16 bp, synthetic construct
  source 1..16: mol_type = other DNA
aatcttccagggccac

SEQ ID NO: 88 - DNA, 16 bp, synthetic construct
  source 1..16: mol_type = other DNA
tcatttctatggaata

SEQ ID NO: 89 - DNA, 16 bp, synthetic construct
  source 1..16: mol_type = other DNA
gtcagtatcccagtgt

SEQ ID NO: 90 - DNA, 16 bp, synthetic construct
  source 1..16: mol_type = other DNA
ggttacagtggaagag

SEQ ID NO: 91 - DNA, 16 bp, synthetic construct
  source 1..16: mol_type = other DNA
tctgggtgttcttacg

SEQ ID NO: 92 - DNA, 16 bp, synthetic construct
  source 1..16: mol_type = other DNA
tttccttgagtagtag

SEQ ID NO: 93 - DNA, 16 bp, synthetic construct
  source 1..16: mol_type = other DNA
tctccttgctgtattt

SEQ ID NO: 94 - DNA, 16 bp, synthetic construct
  source 1..16: mol_type = other DNA
ttgccaatatcaccat

SEQ ID NO: 95 - DNA, 16 bp, synthetic construct
  source 1..16: mol_type = other DNA
caactgaaccacccgt

SEQ ID NO: 96 - DNA, 16 bp, synthetic construct
  source 1..16: mol_type = other DNA
gcacaatatcattaac

SEQ ID NO: 97 - DNA, 16 bp, synthetic construct
  source 1..16: mol_type = other DNA
gactctctgatgatac

SEQ ID NO: 98 - DNA, 16 bp, synthetic construct
  source 1..16: mol_type = other DNA
ctataccatctctcat

SEQ ID NO: 99 - DNA, 16 bp, synthetic construct
  source 1..16: mol_type = other DNA
catcatctataccatc

SEQ ID NO: 100 - DNA, 16 bp, synthetic construct
  source 1..16: mol_type = other DNA
acacatttagcatgac

SEQ ID NO: 101 - DNA, 16 bp, synthetic construct
  source 1..16: mol_type = other DNA
attatatggcaactca

SEQ ID NO: 102 - DNA, 16 bp, synthetic construct
  source 1..16: mol_type = other DNA
gactaatatgcagttt

SEQ ID NO: 103 - DNA, 16 bp, synthetic construct
  source 1..16: mol_type = other DNA
gtcaaattcaagggtt

SEQ ID NO: 104 - DNA, 16 bp, synthetic construct
  source 1..16: mol_type = other DNA
cataaagcatggtgga

SEQ ID NO: 105 - DNA, 16 bp, synthetic construct
  source 1..16: mol_type = other DNA
tagtctctgtcagtta

SEQ ID NO: 106 - DNA, 16 bp, synthetic construct
  source 1..16: mol_type = other DNA
gtacctatagtctctg

SEQ ID NO: 107 - DNA, 16 bp, synthetic construct
  source 1..16: mol_type = other DNA
tcatgtacctatagtc

SEQ ID NO: 108 - DNA, 16 bp, synthetic construct
  source 1..16: mol_type = other DNA
tcttaatttcatgtac

SEQ ID NO: 109 - DNA, 16 bp, synthetic construct
  source 1..16: mol_type = other DNA
accctcaagtctcctg

SEQ ID NO: 110 - DNA, 16 bp, synthetic construct
  source 1..16: mol_type = other DNA
cagatataggactgga

SEQ ID NO: 111 - DNA, 16 bp, synthetic construct
  source 1..16: mol_type = other DNA
gacgcgcctgaaggtt

SEQ ID NO: 112 - DNA, 16 bp, synthetic construct
  source 1..16: mol_type = other DNA
ggacacattggccaca

SEQ ID NO: 113 - DNA, 16 bp, synthetic construct
  source 1..16: mol_type = other DNA
ggccaccacgctgtca

SEQ ID NO: 114 - DNA, 16 bp, synthetic construct
  source 1..16: mol_type = other DNA
tgccaccgtagacacg

SEQ ID NO: 115 - DNA, 16 bp, synthetic construct
  source 1..16: mol_type = other DNA
gctagcctctggattt

SEQ ID NO: 116 - RNA, 20 nt, synthetic construct
  source 1..20: mol_type = other RNA
gataatgtgagaacatgcct

SEQ ID NO: 117 - DNA, 20 bp, synthetic construct
  source 1..20: mol_type = other DNA
aggcatgttctcacattatc

SEQ ID NO: 118 - DNA, 16 bp, synthetic construct
  source 1..16: mol_type = other DNA
ggctactacgccgtca

SEQ ID NO: 119 - DNA, 19 bp, synthetic construct
  source 1..19: mol_type = other DNA
ttttttgcgcggtcctttc

SEQ ID NO: 120 - DNA, 22 bp, synthetic construct
  source 1..22: mol_type = other DNA
gagggaccagagagagcaagac

SEQ ID NO: 121 - DNA, 19 bp, synthetic construct
  source 1..19: mol_type = other DNA
cgccttccgtccgtcggct

SEQ ID NO: 122 - DNA, 21 bp, synthetic construct
  source 1..21: mol_type = other DNA
tggagactctcagggtcgaaa

SEQ ID NO: 123 - DNA, 22 bp, synthetic construct
  source 1..22: mol_type = other DNA
ggcgtttggagtggtagaaatc

SEQ ID NO: 124 - DNA, 20 bp, synthetic construct
  source 1..20: mol_type = other DNA
cggcggcagaccagcatgac

SEQ ID NO: 125 - DNA, 17 bp, synthetic construct
  source 1..17: mol_type = other DNA
gcatgttctcacattat

SEQ ID NO: 126 - DNA, 16 bp, synthetic construct
  source 1..16: mol_type = other DNA
  modified_base 5: mod_base = OTHER; note = uracil
cacctgcgggaagctc

SEQ ID NO: 127 - DNA, 16 bp, synthetic construct
  source 1..16: mol_type = other DNA
  modified_base 5: mod_base = OTHER; note = uracil
agactctcggttccga

SEQ ID NO: 128 - DNA, 16 bp, synthetic construct
  source 1..16: mol_type = other DNA
  modified_base 5: mod_base = OTHER; note = uracil
cttttctatcagtctc

SEQ ID NO: 129 - DNA, 16 bp, synthetic construct
  source 1..16: mol_type = other DNA
  modified_base 5: mod_base = OTHER; note = uracil
cttcttgatgtctttc

SEQ ID NO: 130 - DNA, 16 bp, synthetic construct
  source 1..16: mol_type = other DNA
  modified_base 5: mod_base = OTHER; note = uracil
ggactgaaatagcaga

SEQ ID NO: 131 - DNA, 16 bp, synthetic construct
  source 1..16: mol_type = other DNA
  modified_base 5: mod_base = OTHER; note = uracil
aggctggcccccactg

SEQ ID NO: 132 - DNA, 16 bp, synthetic construct
  source 1..16: mol_type = other DNA
  modified_base 5: mod_base = OTHER; note = uracil
ggtttttgattcttcc

SEQ ID NO: 133 - DNA, 16 bp, synthetic construct
  source 1..16: mol_type = other DNA
  modified_base 5: mod_base = OTHER; note = uracil
gttattgtggttggcg

SEQ ID NO: 134 - DNA, 16 bp, synthetic construct
  source 1..16: mol_type = other DNA
  modified_base 5: mod_base = OTHER; note = uracil
attctgtgtgcactgc

SEQ ID NO: 135 - DNA, 16 bp, synthetic construct
  source 1..16: mol_type = other DNA
  modified_base 5: mod_base = OTHER; note = uracil
ctgtttgagttttctc

SEQ ID NO: 136 - DNA, 16 bp, synthetic construct
  source 1..16: mol_type = other DNA
  modified_base 5: mod_base = OTHER; note = uracil
aatcttccagggccac

SEQ ID NO: 137 - DNA, 16 bp, synthetic construct
  source 1..16: mol_type = other DNA
  modified_base 5: mod_base = OTHER; note = uracil
tcatttctatggaata

SEQ ID NO: 138 - DNA, 16 bp, synthetic construct
  source 1..16: mol_type = other DNA
  modified_base 5: mod_base = OTHER; note = uracil
caactgaaccacccgt

SEQ ID NO: 139 - DNA, 16 bp, synthetic construct
  source 1..16: mol_type = other DNA
  modified_base 5: mod_base = OTHER; note = uracil
attatatggcaactca

SEQ ID NO: 140 - DNA, 16 bp, synthetic construct
  source 1..16: mol_type = other DNA
  modified_base 5: mod_base = OTHER; note = uracil
accctcaagtctcctg

SEQ ID NO: 141 - DNA, 17 bp, synthetic construct
  source 1..17: mol_type = other DNA
ggcatgttctcacatta

SEQ ID NO: 142 - DNA, 16 bp, synthetic construct
  source 1..16: mol_type = other DNA
ggactgaaattgcaga

SEQ ID NO: 143 - DNA, 16 bp, synthetic construct
  source 1..16: mol_type = other DNA
  modified_base 4: mod_base = OTHER; note = uracil
gtctgtgcatctctcc

SEQ ID NO: 144 - DNA, 16 bp, synthetic construct
  source 1..16: mol_type = other DNA
acatcttcagatcatt

SEQ ID NO: 145 - DNA, 16 bp, synthetic construct
  source 1..16: mol_type = other DNA
  modified_base 4: mod_base = OTHER; note = uracil
tagtctctgtcagtta

SEQ ID NO: 146 - DNA, 17 bp, synthetic construct
  source 1..17: mol_type = other DNA
  modified_base 6: mod_base = OTHER; note = uracil
gcatgttctcacattat

SEQ ID NO: 147 - DNA, 16 bp, synthetic construct
  source 1..16: mol_type = other DNA
acaaggacaccaagat

SEQ ID NO: 148 - DNA, 25 bp, synthetic construct
  source 1..25: mol_type = other DNA
gggattacagagatcgtgactgatt

SEQ ID NO: 149 - DNA, 20 bp, synthetic construct
  source 1..20: mol_type = other DNA
tgcagctggaagaaccaaaa

SEQ ID NO: 150 - DNA, 30 bp, synthetic construct
  source 1..30: mol_type = other DNA
cagagtaaaatacccattccagctcctggg

SEQ ID NO: 151 - DNA, 21 bp, synthetic construct
  source 1..21: mol_type = other DNA
gattcgtcagctttgccaagt

SEQ ID NO: 152 - DNA, 22 bp, synthetic construct
  source 1..22: mol_type = other DNA
cgtctgttcagttgtcaatgca

SEQ ID NO: 153 - DNA, 29 bp, synthetic construct
  source 1..29: mol_type = other DNA
tctgggcctcaaggataacaacatcgttt

SEQ ID NO: 154 - DNA, 16 bp, synthetic construct
  source 1..16: mol_type = other DNA
  modified_base 6: mod_base = OTHER; note = uracil
gttattgtggttggcg

(19) United States
(12) Patent Application Publication
(10) Pub. No.: US 2025/0354135 A1
(43) Pub. Date: Nov. 20, 2025

(54) Modified Compounds and Uses Thereof

(71) Applicant: Ionis Pharmaceuticals, Inc., Carlsbad, CA (US)

(72) Inventors: Punit P. Seth, Carlsbad, CA (US); Michael Oestergaard, Carlsbad, CA (US); Michael T. Migawa, Carlsbad, CA (US); Xue-hai Liang, Del Mar, CA (US); Wen Shen, Carlsbad, CA (US); Stanley T. Crooke, Carlsbad, CA (US); Eric E. Swayze, Encinitas, CA (US)

(73) Assignee: Ionis Pharmaceuticals, Inc., Carlsbad, CA (US)

(21) Appl. No.: 18/984,293

(22) Filed: Dec. 17, 2024

Related U.S. Application Data
(63) Continuation of application No. 17/697,577, filed on Mar. 17, 2022, now Pat. No. 12,234,447.
Application No. 17/697,577 is a continuation of application No. 16/968,701, filed on Aug. 10, 2020, now Pat. No. 11,332,733.
(60) Provisional application No. 62/629,632, filed on Feb. 12, 2018.
(60) Provisional application No. 62/686,632, filed on Jun. 18, 2018.
(60) Provisional application No. 62/713,698, filed on Aug. 2, 2018.
(60) Provisional application No. 62/739,088, filed on Sep. 28, 2018.
(60) Provisional application No. 62/742,265, filed on Oct. 5, 2018.
(60) Provisional application No. 62/746,511, filed on Oct. 16, 2018.

(51) Int. Cl. C12N 15/10; C12N 15/113

(52) U.S. Cl. CPC C12N 15/10; C12N 15/113; C12N 2310/315; C12N 2310/3231

(57) ABSTRACT

The present disclosure provides oligomeric compound comprising a modified oligonucleotide having a central region comprising one or more modifications. In certain embodiments, the present disclosure provides oligomeric compounds having an improved therapeutic index or an increased maximum tolerated dose.

### SEQUENCE LISTING

The present application contains a Sequence Listing which has been submitted electronically in XML format. Said XML copy, created on Jan. 20, 2023, is named “CORE0148SEQ.xml” and is 410,463 bytes in size. The information in the electronic format of the sequence listing is incorporated herein by reference in its entirety.

### FIELD

The present disclosure provides oligomeric compounds comprising a modified oligonucleotide having a central region comprising one or more modifications. In certain embodiments, the present disclosure provides oligomeric compounds having an improved therapeutic index or an increased maximum tolerated dose.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics, or affinity for a target nucleic acid.

### SUMMARY

The present disclosure provides oligomeric compounds and methods of using oligomeric compounds that comprise a modified oligonucleotide consisting of 14-23 linked nucleosides, wherein the modified oligonucleotide comprises a gapmer consisting of a 5′-region, a central region, and a 3′-region wherein:
the 5′-region consists of 2-5 linked modified nucleosides, wherein each nucleoside of the 5′-region comprises a 2′-modified furanosyl sugar moiety;
the 3′-region consists of 1-5 linked modified nucleosides, wherein each nucleoside of the 3′-region comprises a 2′-modified furanosyl sugar moiety;
the central region consists of 7-10 linked nucleosides, where each nucleoside of the central region comprises a sugar moiety selected from an unmodified 2′-β-D-deoxyribosyl sugar moiety and a modified sugar moiety; wherein
the 3′- and 5′-most nucleosides of the central region each comprise a sugar moiety independently selected from a 2′-deoxyfuranosyl sugar moiety or a sugar surrogate; and wherein
the central region comprises:
   at least one altered nucleotide, comprising a modified internucleoside linkage other than phosphorothioate and/or a modified nucleobase other than 5-methylcytosine and/or a modified sugar moiety; and
   at least 6 nucleosides each comprising an unmodified 2′-β-D-deoxyribosyl sugar moiety.

In certain embodiments, oligomeric compounds are provided comprising a single conjugate group linked to the 5′-end. In certain embodiments, oligomeric compounds are provided comprising a single conjugate group linked to the 3′-end.

In certain embodiments, the oligomeric compounds provided herein have an increased maximum tolerated dose when administered to an animal compared to an otherwise identical oligomeric compound except that the otherwise identical oligomeric compound lacks the altered nucleotide in the central region.

In certain embodiments, the oligomeric compounds provided herein have an increased therapeutic index compared to an otherwise identical oligomeric compound except that the otherwise identical oligomeric compound lacks the alterered nucleotide in the central region.

In certain embodiments, methods of inhibiting target RNA are provided comprising contacting one or more cells, a tissue or an animal with an oligomeric compound as provided herein wherein said oligomeric compound is complementary to a target RNA.

In certain embodiments, the cells are in a human. In certain embodiments, the target RNA is human RNA. In certain embodiments, the target is human mRNA. In certain embodiments, the target RNA is cleaved, thereby inhibiting its function.

In certain embodiments, in vitro methods of inhibiting gene expression are provided comprising contacting one or more cells or a tissue with an oligomeric compound as provided herein.

In certain embodiments, oligomeric compounds are provided for use in an in vivo method of inhibiting gene expression wherein the method comprises contacting one or more cells, a tissue or an animal with an oligomeric compound as provided herein.

In certain embodiments, oligomeric compounds are provided for use in medical therapy.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

As used herein, “2′-deoxyfuranosyl sugar moiety” or “2′-deoxyfuranosyl sugar” means a furanosyl sugar moiety having two hydrogens at the 2′-position. 2′-deoxyfuranosyl sugar moieties may be unmodified or modified and may be substituted at positions other than the 2′-position or unsubstituted. A β-D-2′-deoxyribosyl sugar moiety or 2′-β-D-deoxyribosyl sugar moiety in the context of an oligonucleotide is an unsubstituted, unmodified 2′-deoxyfuranosyl and is found in naturally occurring deoxyribonucleic acids (DNA).

As used herein, “2′-modified” in reference to a furanosyl sugar moiety or nucleoside comprising a furanosyl sugar moiety means the furanosyl sugar moiety comprises a substituent other than H or OH at the 2′-position of the furanosyl sugar moiety. 2′-modified furanosyl sugar moieties include non-bicyclic and bicyclic sugar moieties and may comprise, but are not required to comprise, additional substituents at other positions of the furanosyl sugar moiety.

As used herein, “2′-ribo-F” indicates a 2′-fluororibose.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “4′-modified” in reference to a furanosyl sugar moiety or nucleoside comprising a furanosyl sugar moiety means the furanosyl sugar moiety comprises a substituent other than H at the 4′-position of the furanosyl sugar moiety. 4′-modified furanosyl sugar moieties include non-bicyclic and bicyclic sugar moieties and may but are not required to comprise additional substituents at other positions of the furanosyl sugar moiety.

As used herein, “4′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 4′-position and is a non-bicyclic furanosyl sugar moiety. 4′-substituted furanosyl sugar moieties do not comprise additional substituents at other positions of the furanosyl sugar moiety other than a nucleobase and/or internucleoside linkage(s) when in the context of an oligonucleotide.

As used herein, “5′-modified” in reference to a furanosyl sugar moiety or nucleoside comprising a furanosyl sugar moiety means the furanosyl sugar moiety comprises a substituent other than H at the 5′-position of the furanosyl sugar moiety. 5′-modified furanosyl sugar moieties may but are not required to comprise additional substituents at other positions of the furanosyl sugar moiety.

As used herein, “5′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 5′-position and is a non-bicyclic furanosyl sugar moiety. 5′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration.

As used herein, “administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel, sequentially, separate, or simultaneous administration.

As used herein, “ALT” means alanine aminotransferase. As used herein, “AST” means aspartate transaminase. In certain embodiments, plasma levels of ALT and AST in a subject are measured in units per liter. As used herein, “units per liter” in the context of plasma ALT or plasma AST levels means international units per liter, the standard units for measurement of plasma ALT or plasma AST levels used by those of ordinary skill in the medical arts.

As used herein, “animal” refers to a human or non-human animal, including, but not limited to, mice, rats, rabbits, dogs, cats, pigs, and non-human primates, including, but not limited to, monkeys and chimpanzees.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified furanosyl sugar moiety. In certain embodiments, the bicyclic sugar moiety does not comprise a furanosyl moiety.

As used herein, a “central nervous system target” is a target RNA that is expressed in the central nervous system.

As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, a “cEt nucleoside” or “cEt nucleotide” is a nucleoside or nucleotide comprising a cEt.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups may comprise a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” or “adjacent” in the context of an oligonucleotide refers to nucleosides, nucleobases, sugar moieties, or internucleoside linkages that are immediately adjacent to each other independent of the other moieties of the oligonucleotide. For example, “contiguous nucleobases” means nucleobases that are immediately adjacent to each other in a sequence. Moieties that are “directly linked” are immediately adjacent to each other and not separated by any other type of moiety.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “effective amount” means the amount of compound sufficient to effectuate a desired physiological outcome in a subject in need of the compound. The effective amount may vary among subjects depending on the health and physical condition of the subject to be treated, the taxonomic group of the subjects to be treated, the formulation of the composition, assessment of the subject's medical condition, and other relevant factors.

As used herein, “efficacy” means the ability to produce a desired effect.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. Herein, the nucleosides of the 5′-region and 3′-region each comprise a 2′-modified furanosyl sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”.

As used herein, “hepatotoxic” in the context of a mouse means a plasma ALT level that is above 300 units per liter. Hepatotoxicity of an oligomeric compound or parent oligomeric compound that is administered to a mouse is determined by measuring the plasma ALT level of the mouse 24 hours to 2 weeks following at least one dose of 1-150 mg/kg of the compound.

As used herein, “hepatotoxic” in the context of a human means a plasma ALT level that is above 150 units per liter. Hepatotoxicity of an oligomeric compound or parent oligomeric compound that is administered to a human is determined by measuring the plasma ALT level of the human 24 hours to 2 weeks following at least one dose of 10-300 mg of the compound.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0.

As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “LICA-1” is a conjugate group that is represented by the formula:

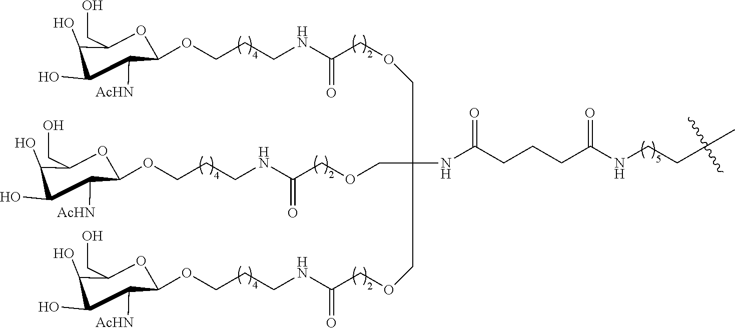

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic sugar” or “non-bicyclic sugar moiety” means a sugar moiety that comprises fewer than 2 rings. Substituents of modified, non-bicyclic sugar moieties do not form a bridge between two atoms of the sugar moiety to form a second ring.

As used herein, “linked nucleosides” are nucleosides that are connected in a continuous sequence (i.e. no additional nucleosides are present between those that are linked).

As used herein, “liver target” is a target RNA expressed in the liver wherein modulation of the expression of the target RNA in the liver is desired for therapeutic benefit. In certain embodiments, a liver target is expressed in tissues other than the liver as well as in the liver. As used herein, modulation of the expression of a target RNA that is “not a liver target” is desired in a tissue that is not the liver for therapeutic benefit. In certain embodiments, a target RNA that is not a liver target is expressed in the liver and is modulated by an oligomeric compound in therapy.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring.

As used herein, “MOP” or “methoxypropyl internucleoside linkage” means the alkyl phosphonate internucleoside bond shown below:

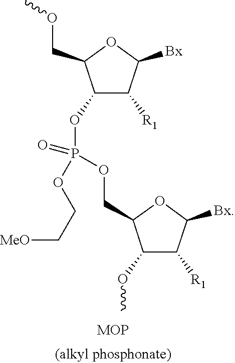

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “parent oligomeric compound” in the context of an oligomeric compound comprising at least one modification in the central region other than phosphorothioate or 5-methylcytosine means an oligomeric compound that is identical to the oligomeric compound comprising the at least one modification in the central region except that the parent oligomeric compound does not comprise at least one modified internucleoside linkage other than phosphorothioate and/or at least one modified nucleoside comprising a modified nucleobase other than 5-methylcytosine and/or a modified sugar moiety. A parent oligomeric compound and its counterpart oligomeric compound comprising at least one modification in the central region have identical nucleobase sequences or differ in nucleobase sequence only due to inclusion of a modified nucleobase other than 5-methylcytosine in the oligomeric compound comprising at least one modification in the central region.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “standard cell assay” means any of the assays described in Examples 1-9, and reasonable variations thereof.

As used herein, “standard in vitro activity assay” means a procedure, as described in Example 1 herein, wherein expression is measured by RT-PCR in cultured cells expressing the target RNA following administration of an oligomeric compound to the cultured cells.

As used herein, “standard in vitro cytotoxicity assay” means a procedure, as described in Example 8 herein, wherein activation of caspases 3 and 7 is measured in cultured 3T3-L1 cells following administration of an oligomeric compound to the cells.

As used herein, “standard in vivo experiment” means the procedure described in Example 10 and reasonable variations thereof.

As used herein, “stereorandom” in the context of a compound or moiety comprising a chiral center means the chiral center has a random stereochemical configuration. For example, in a population of molecules of identical formula comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not have a stereoconfiguration other than β-D-ribosyl. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “susceptible” in the context of a disease, disorder, condition, or symptom such as degeneration, damage, or elevated apoptosis means that a subject has a higher risk than the average risk for the general population for the disease, disorder, condition, or symptom.

As used herein, “target nucleic acid,” “target RNA,” “target RNA transcript” and “nucleic acid target” mean a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound.

In certain embodiments, the target RNA is an RNA present in the species to which an oligomeric compound is administered. As used herein, a “liver target” is a target RNA that is expressed in the liver, and modulation of expression of the target RNA in the liver provides a therapeutic effect. As used herein a “central nervous system target” is a target RNA that is expressed in the central nervous system, and modulation of expression of the target RNA in the central nervous system provides a therapeutic effect.

The present disclosure provides certain individual cellular, tissue, or organ targets. For example, a “macrophage target” or a “liver target.” For each such individual target, modulation of the expression of the target RNA in the individual cellular, tissue, or organ target is desired for therapeutic benefit. In certain embodiments, modulation of the target RNA in an individual cellular, tissue, or organ target provides a therapeutic effect. In certain embodiments, a cellular, tissue, or organ target is expressed in tissues other than in a particular type of cell, tissue, or organ as well as being expressed in a particular type of cell, tissue, or organ. For example, certain target RNAs may be expressed in both a macrophage and a hepatocyte.

As used herein, “therapeutically effective amount” means an amount of a compound, pharmaceutical agent, or composition that provides a therapeutic benefit to a subject.

As used herein, “treat” refers to administering a compound or pharmaceutical composition to an animal in order to effect an alteration or improvement of a disease, disorder, or condition in the animal.

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay, e.g. the assay of Example 12 or Example 1. In certain embodiments, the maximum tolerated dose is the highest dose of a modified oligonucleotide that does not cause caspase elevation of greater than 30,000 RLU as measured by a standard assay, e.g. the assay of Example 13, Example 8, or Example 4.

As used herein, “DNA isomer” means a nucleoside that comprises a modified sugar moiety that is a stereoisomer of β-D-2′-deoxyribosyl. As used herein, a “DNA isomer” does not include β-D-2′-deoxyribosyl nucleosides. Seven such isomers of 2′-β-D-deoxyribosyl exist: 2′-β-D-deoxyxylosyl (β-D-XNA), 2′-α-D-deoxyribosyl (α-D-DNA), 2′-α-L-deoxyribosyl (α-L-DNA), 2′-β-L-deoxyribosyl (β-L-DNA), 2′-α-D-deoxyxylosyl (α-L-XNA), 1, 2′-α-L-deoxyxylosyl (α-L-XNA), 2′-β-L-deoxyxylosyl (β-L-XNA). In certain embodiments, a DNA isomer is 2′-α-D-deoxyribosyl, 2′-α-L-deoxyribosyl, 2′-β-L-deoxyribosyl, or 2′-β-D-deoxyxylosyl sugar moiety. As used herein, “DNA isomer” does not include any nonfuranosyl sugar moieties.

As used herein, “DNA nucleoside” means a nucleoside comprising a 2′-H(H) β-D-2′-deoxyribosyl sugar moiety, as found in naturally-occuring DNA. A “DNA nucleoside” may comprise a modified nucleobase or a uracil nucleobase. A DNA nucleoside may be linked to adjacent nucleosides through unmodified phosphodiester internucleoside linkages or through modified internucleoside linkages.

As used herein, a “2′-modified DNA isomer” means a nucleoside that comprises a modified sugar moiety that is selected from 2′-β-D-deoxyxylosyl (β-D-XNA), 2′-α-D-deoxyribosyl (α-D-DNA), 2′-α-L-deoxyribosyl (α-L-DNA), 2′-β-L-deoxyribosyl (β-L-DNA), 2′-α-D-deoxyxylosyl (α-L-XNA), 1, 2′-α-L-deoxyxylosyl (α-L-XNA), 2′-β-L-deoxyxylosyl (β-L-XNA), and that further comprises a 2′-substituent. In certain embodiments, the 2′-substituent is fluoro, alkoxy, or C₁-C₁₀ alkyl.

As used herein, “DNA mimic” means a nucleoside other than a DNA nucleoside wherein the nucleobase is directly linked to a carbon atom of a ring bound to a second carbon atom within the ring, wherein the second carbon atom comprises a bond to at least one hydrogen atom, wherein the nucleobase and at least one hydrogen atom are trans to one another relative to the bond between the two carbon atoms. In certain embodiments, a DNA mimic comprises a structure represented by the formula:

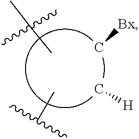

wherein Bx is a heterocylic base moiety, the ring contains 5-7 members, and the ring is attached at two positions to a hydroxyl, a phosphate, an internucleoside linking group, or a conjugate linker.

As used herein, a “standard RNase H cleavage assay” is an assay wherein a heteroduplex of the modified oligonucleotide and a complementary strand of unmodified RNA are incubated with each other to form a heteroduplex, and are then incubated with RNase H1 for specified time points before being analyzed on a polyacrylamide gel.

As used herein, a modified nucleoside “supports RNase H cleavage” when incorporated into an oligonucleotide if RNase H cleavage of the complementary RNA is observed within two nucleobases of the modified nucleoside in a standard RNase H cleavage assay.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered. In certain embodiments, therapeutic index is the ratio of the amount of modulation of a target nucleic acid by a modified oligonucleotide compared to ALT elevation, wherein the ALT elevation is measured by a standard assay, e.g. the assay of Example 12 or Example 1. In certain embodiments, therapeutic index is the ratio of the amount of modulation of a target nucleic acid by a modified oligonucleotide compared to caspase elevation, wherein the caspase elevation is measured by a standard assay, e.g. the assay of Example 13, Example 8, or Example 4.

As used herein, an “altered nucleotide” is a nucleotide that comprises one or more modifications relative to a nucleotide comprising a 2′-β-D-deoxyribosyl sugar moiety, a nucleobase selected from adenine (A), thymine (T), cytosine (C), 5-methyl cytosine (^{m}C), uracil (U), or guanine (G), and a 5′ to 3′ internucleoside linkage selected from phosphodiester or stereorandom phosphorothioate. In certain embodiments, the altered nucleotide is an altered nucleoside attached to a phosphorothioate or phosphodiester internucleoside linkage. In certain embodiments, the altered nucleotide comprises a 2′-modified sugar moiety, or is a “2′-altered nucleotide”. In certain embodiments, the altered nucleotide comprises a modified internucleoside linking group, and is a “linkage-altered nucleotide”. Herein, a linkage-altered nucleotide comprises an internucleoside linking group other than phosphodiester or phosphorothioate attached to the 3′ carbon of the sugar moiety, or the equivalent position for a sugar surrogate. The nucleotide on the 5′-end of an internucleoside linking group other than phosphodiester or phoshporothioate is not an “altered nucleotide”, as used herein.

Certain embodiments are described in the numbered embodiments below:
1. An oligomeric compound comprising a modified oligonucleotide consisting of 14-23 linked nucleosides, wherein the modified oligonucleotide comprises a gapmer consisting of a 5′-region, a central region, and a 3′-region wherein:
   the 5′-region consists of 2-5 linked modified nucleosides, wherein each nucleoside of the 5′-region comprises a 2′-modified furanosyl sugar moiety;
   the 3′-region consists of 1-5 linked modified nucleosides, wherein each nucleoside of the 3′-region comprises a 2′-modified furanosyl sugar moiety;
   the central region consists of 7-10 linked nucleosides, where each nucleoside of the central region comprises a sugar moiety selected from an unmodified 2′-β-D-deoxyribosyl sugar moiety and a modified sugar moiety; wherein
   the 3′- and 5′-most nucleosides of the central region each comprise a sugar moiety independently selected from a 2′-deoxyfuranosyl sugar moiety or a sugar surrogate; and wherein the central region comprises:
   at least one modified internucleoside linkage other than phosphorothioate and/or at least one modified nucleoside comprising a modified nucleobase other than 5-methylcytosine and/or a modified sugar moiety; and
   at least 6 nucleosides each comprising an unmodified 2′-β-D-deoxyribosyl sugar moiety.
2. The oligomeric compound of embodiment 1, wherein the 5′-region consists of 2-4 linked nucleosides.
3. The oligomeric compound of embodiment 1, wherein the 5′-region consists of 2 linked nucleosides.
4. The oligomeric compound of embodiment 1, wherein the 5′-region consists of 3 linked nucleosides.
5. The oligomeric compound of embodiment 1, wherein the 5′-region consists of 4 linked nucleosides.
6. The oligomeric compound of embodiment 1, wherein the 5′-region consists of 5 linked nucleosides.
7. The oligomeric compound of any of embodiments 1-6, wherein each nucleoside of the 5′-region comprises a 2′-modified ribosyl sugar moiety.
8. The oligomeric compound of any of embodiments 1-7, wherein at least one nucleoside of the 5′-region comprises a bicyclic furanosyl sugar moiety.
9. The oligomeric compound of any of embodiments 1-8, wherein each nucleoside of the 5′-region comprises a bicyclic furanosyl sugar moiety.
10. The oligomeric compound of any of embodiments 1-8, wherein at least one nucleoside of the 5′-region comprises a non-bicyclic sugar moiety.
11. The oligomeric compound of embodiment 10, wherein the non-bicyclic sugar moiety of the at least one nucleoside of the 5′-region is a 2′-substituted ribosyl sugar moiety.
12. The oligomeric compound of any of embodiments 1-7 or 10-11, wherein each nucleoside of the 5′-region comprises a non-bicyclic sugar moiety.
13. The oligomeric compound of embodiment 12, wherein each nucleoside of the 5′-region comprises a 2′-substituted ribosyl sugar moiety.
14. The oligomeric compound of any of embodiments 1-7, wherein each nucleoside of the 5′-region comprises a 2′-modified furanosyl sugar moiety independently selected from a bicyclic sugar moiety and a non-bicyclic sugar moiety, 2′-substituted ribosyl sugar moiety.
15. The oligomeric compound of any of embodiments 8-11 or 14, wherein each bicyclic sugar moiety of the 5′-region is selected from among cEt, LNA, and ENA.
16. The oligomeric compound of any of embodiments 10-14, wherein each nonbicyclic sugar moiety of the 5′-region has a 2′-substituent selected from among 2′-MOE, 2′-OMe, and 2′-NMA.
17. The oligomeric compound of any of embodiments 1-16, wherein none of the nucleosides of the 5′-region comprise a sugar moiety having a 2′-F substituent.
18. The oligomeric compound of any of embodiments 1-17, wherein each nucleobase of the 5′-region is independently selected from among thymine, uracil, guanine, cytosine, 5-methylcytosine, and adenine.
19. The oligomeric compound of any of embodiments 1-18, wherein each internucleoside linkage of the 5′-region is selected from among phosphodiester and phosophorothioate internucleoside linkages.
20. The oligomeric compound of any of embodiments 1-19, wherein the 3′-region consists of 2-4 linked nucleosides.
21. The oligomeric compound of any of embodiments 1-19, wherein the 3′-region consists of 1 nucleoside.
22. The oligomeric compound of any of embodiments 1-19, wherein the 3′-region consists of 2 linked nucleosides.
23. The oligomeric compound of any of embodiments 1-19, wherein the 3′-region consists of 3 linked nucleosides.
24. The oligomeric compound of any of embodiments 1-19, wherein the 3′-region consists of 4 linked nucleosides.
25. The oligomeric compound of any of embodiments 1-19, wherein the 3′-region consists of 5 linked nucleosides.
26. The oligomeric compound of any of embodiments 1-25, wherein each nucleoside of the 3′-region comprises a 2′-modified ribosyl sugar moiety.
27. The oligomeric compound of any of embodiments 1-26, wherein at least one nucleoside of the 3′-region comprises a bicyclic furanosyl sugar moiety.
28. The oligomeric compound of any of embodiments 1-27, wherein each nucleoside of the 3′-region comprises a bicyclic furanosyl sugar moiety.
29. The oligomeric compound of any of embodiments 1-27, wherein at least one nucleoside of the 3′-region comprises a non-bicyclic sugar moiety.
30. The oligomeric compound of embodiment 29, wherein the non-bicyclic sugar moiety of the at least one nucleoside of the 3′-region is a 2′-substituted ribosyl sugar moiety.
31. The oligomeric compound of any of embodiments 1-26 or 29-30, wherein each nucleoside of the 3′-region comprises a non-bicyclic sugar moiety.
32. The oligomeric compound of embodiment 31, wherein each nucleoside of the 3′-region comprises a 2′-substituted ribosyl sugar moiety.
33. The oligomeric compound of any of embodiments 1-26, wherein each nucleoside of the 3′-region comprises a 2′-modified furanosyl sugar moiety independently selected from a bicyclic sugar moiety and a non-bicyclic sugar moiety, 2′-substituted ribosyl sugar moiety.
34. The oligomeric compound of any of embodiments 27-30 or 33, wherein each bicyclic sugar moiety of the 3′-region is selected from among cEt, LNA, and ENA.
35. The oligomeric compound of any of embodiments 29-33, wherein each nonbicyclic sugar moiety of the 3′-region has a 2′-substituent selected from among 2′-MOE, 2′-OMe, and 2′-NMA.
36. The oligomeric compound of any of embodiments 1-35, wherein none of the nucleosides of the 3′-region comprise a sugar moiety having a 2′-F substituent.
37. The oligomeric compound of any of embodiments 1-36, wherein each nucleobase of the 3′-region is independently selected from among thymine, uracil, guanine, cytosine, 5-methylcytosine, and adenine.
38. The oligomeric compound of any of embodiments 1-37, wherein each internucleoside linkage of the 3′-region is selected from among phosphodiester and phosophorothioate internucleoside linkages.
39. The oligomeric compound of any of embodiments 1-38, wherein the central region consists of 7 linked nucleosides.
40. The oligomeric compound of any of embodiments 1-38, wherein the central region consists of 8 linked nucleosides.
41. The oligomeric compound of any of embodiments 1-38, wherein the central region consists of 9 linked nucleosides.
42. The oligomeric compound of any of embodiments 1-38, wherein the central region consists of 10 linked nucleosides.
43. The oligomeric compound of any of embodiments 1-42, wherein each of the two internucleoside linkages connecting the central region to the 5′-region and 3′-region are independently selected from among phosphosdiester and phosphorothioate internucleoside linkages.
44. The oligomeric compound of any of embodiments 1-43, wherein the modified oligonucleotide consists of the gapmer.
45. The oligomeric compound of any of embodiments 1-43, comprising a conjugate group.
46. The oligomeric compound of any of embodiments 1-43 or 45, wherein the modified oligonucleotide comprises 1-3 linker nucleosides.
47. The oligomeric compound of embodiment 46, wherein the linker nucleosides are linked to the 5′-end or the 3′-end of the gapmer.
48. The oligomeric compound of any of embodiments 45-47, wherein the conjugate group comprises GalNAc.
49. The oligomeric compound of any of embodiments 45-47, comprising LICA-1.
50. The oligomeric compound of any of embodiments 1-49, wherein the central region comprises one, and no more than one, modified sugar moiety.
51. The oligomeric compound of embodiment 50, wherein the each internucleoside linkage within the central region is selected from among phosphodiester and phosophorothioate internucleoside linkages.
52. The oligomeric compound of any of embodiments 50-51, wherein each nucleobase of the central region is independently selected from among thymine, uracil, guanine, cytosine, 5-methylcytosine, and adenine.
53. The oligomeric compound of any of embodiments 1-50 or 52, wherein the central region comprises one, and no more than one, modified internucleoside linkage other than phosphorothioate.
54. The oligomeric compound of embodiment 53, wherein the modified internucleoside linkage other than phosphorothioate contains phosphorus.
55. The oligomeric compound of any of embodiments 53-54, wherein the modified internucleoside linkage other than phosphorothioate is a neutral internucleoside linkage.
56. The oligomeric compound of any of embodiments 1-50 or 52, wherein the central region comprises two, and no more than two, modified internucleoside linkages other than phosphorothioate.
57. The oligomeric compound of embodiment 56, wherein the two modified internucleoside linkages other than phosphorothioate each contain phosphorus.
58. The oligomeric compound of any of embodiments 56-57, wherein at least one of the modified internucleoside linkages other than phosphorothioate is a neutral internucleoside linkage.
59. The oligomeric compound of any of embodiments 56-57, wherein the two modified internucleoside linkages other than phosphorothioate are neutral internucleoside linkages.
60. The oligomeric compound of any of embodiments 1-51 or 53-59, wherein the central region comprises one, and no more than one, modified nucleobase other than 5-methylcytosine.
61. The oligomeric compound of any of embodiments 1-60, wherein each of the unmodified sugar moieties of the central region are 2′-β-D-deoxyribosyl sugar moieties.
62. The oligomeric compound of any of embodiments 50-61, wherein the one modified sugar moiety of the central region is at one of positions 2-9 of the central region.
63. The oligomeric compound of any of embodiments 50-61, wherein the one modified sugar moiety of the central region is at one of positions 1-6 of the central region.
64. The oligomeric compound of any of embodiments 50-61, wherein the one modified sugar moiety of the central region is at one of positions 1-4 of the central region.
65. The oligomeric compound of any of embodiments 50-61, wherein the one modified sugar moiety of the central region is at one of positions 2-4 of the central region.
66. The oligomeric compound of any of embodiments 50-61, wherein the one modified sugar moiety of the central region is at one of positions 3-4 of the central region.
67. The oligomeric compound of any of embodiments 50-66, wherein the one modified sugar moiety of the central region is a sugar surrogate, a bicyclic furanosyl sugar moiety, a non-bicyclic 2′-modified furanosyl sugar moiety, a non-bicyclic 4′-modified furanosyl sugar moiety, a non-bicyclic 5′-modified furanosyl sugar moiety, or a modified 2′-deoxyfuranosyl sugar moiety.
68. The oligomeric compound of embodiment 67, wherein the one modified sugar moiety of the central region is a bicyclic ribosyl sugar moiety, a non-bicyclic 2′-modified ribosyl sugar moiety, a non-bicyclic 4′-modified 2′-deoxyribosyl sugar moiety, a non-bicyclic 5′-modified 2′-deoxyribosyl sugar moiety, or a modified 2′-deoxyfuranosyl sugar moiety.
69. The oligomeric compound of embodiment 68, wherein the one modified sugar moiety of the central region is a bicyclic ribosyl sugar moiety, a non-bicyclic 2′-substituted ribosyl sugar moiety, a non-bicyclic 4′-substituted 2′-deoxyribosyl sugar moiety, a non-bicyclic 5′-substituted 2′-deoxyribosyl sugar moiety, or a modified, unsubstituted 2′-deoxyfuranosyl sugar moiety.
70. The oligomeric compound of any of embodiments 67-69, wherein the one modified sugar moiety of the central region is a 2′-substituted ribosyl sugar moiety.
71. The oligomeric compound of embodiment 70, wherein the 2′-substituted ribosyl sugar moiety is a 2′-F, 2′-MOE, or 2′-O-methyl substituted sugar moiety.
72. The oligomeric compound of any of embodiments 67-69, wherein the one modified sugar moiety of the central region is a 4′-alkyl substituted 2′-deoxyribosyl sugar moiety.
73. The oligomeric compound of embodiment 72, wherein the 4′-alkyl substituted ribosyl sugar moiety is a 4′-methyl substituted 2′-deoxyribosyl sugar moiety.
74. The oligomeric compound of any of embodiments 67-69, wherein the one modified sugar moiety of the central region is a 5′-alkyl substituted 2′-deoxyribosyl sugar moiety.
75. The oligomeric compound of embodiment 74, wherein the 5′-alkyl substituted ribosyl sugar moiety is a 5′-methyl, 5′-ethyl, or 5′-allyl substituted 2′-deoxyribosyl sugar moiety.
76. The oligomeric compound of any of embodiments 67-69, wherein the one modified sugar moiety of the central region is a modified, unsubstituted 2′-deoxyfuranosyl sugar moiety.
77. The oligomeric compound of embodiment 76, wherein the modified, unsubstituted 2′-deoxyfuranosyl sugar moiety is an unsubstituted α-D-2′-deoxyribosyl, α-L-2′-deoxyribosyl, β-L-2′-deoxyribosyl, or β-D-2′-deoxyxylosyl sugar moiety.
78. The oligomeric compound of any of embodiments 67-69, wherein the one modified sugar moiety of the central region is a bicyclic ribosyl sugar moiety.
79. The oligomeric compound of embodiment 78, wherein the bicyclic ribosyl sugar moiety is cEt, LNA, or ENA.
80. The oligomeric compound of any of embodiments 67-69, wherein the one modified sugar moiety of the central region is a morpholino, cEt, 2′-F, 2′-O-Methyl, 2′-MOE, 4′-Methyl, 5′-Methyl, 5′-allyl, 5′-ethyl, β-L-2′-deoxyribosyl, α-D-2′-deoxyribosyl, β-D-2′-deoxyxylosyl, or α-L-2′-deoxyribosyl sugar moiety.
81. The oligomeric compound of embodiment 62, wherein the one modified sugar moiety of the central region is a 2′-O-Methyl substituted ribosyl, unsubstituted α-D-2′-deoxyribosyl, unsubstituted α-L-2′-deoxyribosyl, or unsubstituted β-L-2′-deoxyribosyl sugar moiety.
82. The oligomeric compound of embodiment 81, wherein the one modified sugar moiety of the central region is a 2′-O-Methyl substituted ribosyl or unsubstituted β-L-2′-deoxyribosyl sugar moiety.
83. The oligomeric compound of embodiment 63, wherein the one modified sugar moiety of the central region is a morpholino, 2′-O-Methyl substituted ribosyl, unsubstituted α-D-2′-deoxyribosyl, or unsubstituted β-L-2′-deoxyribosyl sugar moiety sugar moiety.
84. The oligomeric compound of embodiment 64, wherein the one modified sugar moiety of the central region is a morpholino, unsubstituted α-D-2′-deoxyribosyl, or unsubstituted β-L-2′-deoxyribosyl sugar moiety sugar moiety.
85. The oligomeric compound of embodiment 65, wherein the one modified sugar moiety of the central region is a 2′-F substituted ribosyl, 2′-MOE substituted ribosyl, 2′-O-Methyl substituted ribosyl, 5′-(S)-Methyl substituted 2′-deoxyribosyl, 5′-allyl substituted 2′-deoxyribosyl, unsubstituted α-D-2′-deoxyribosyl, or cEt bicyclic sugar moiety.
86. The oligomeric compound of embodiment 85, wherein the one modified sugar moiety of the central region is a 2′-MOE substituted ribosyl, 2′-O-Methyl substituted ribosyl, or 5′-allyl substituted 2′-deoxyribosyl sugar moiety.
87. The oligomeric compound of embodiment 85 or 86, wherein the 5′-allyl substituted ribosyl sugar moiety is stereorandom at the 5′-position of the modified sugar moiety.
88. The oligomeric compound of embodiment 66, wherein the one modified sugar moiety of the central region is a 2′-F substituted ribosyl, 2′-MOE substituted ribosyl, 2′-O-Methyl substituted ribosyl, 4′-Methyl substituted 2′-deoxyribosyl, 5′-(S)-Methyl substituted 2′-deoxyribosyl, 5′-(R)-Methyl substituted 2′-deoxyribosyl, 5′-stereorandom Methyl substituted 2′-deoxyribosyl, 5′-(S)-Ethyl substituted 2′-deoxyribosyl, 5′-(R)-Ethyl substituted 2′-deoxyribosyl, 5′-stereorandom Ethyl 2′-deoxyribosyl, 5′-(S)-Allyl substituted 2′-deoxyribosyl, 5′-(R)-Allyl substituted 2′-deoxyribosyl, 5′-stereorandom allyl substituted 2′-deoxyribosyl, unsubstituted α-D-2′-deoxyribosyl, unsubstituted α-L-2′-deoxyribosyl, unsubstituted β-L-2′-deoxyribosyl, cEt, or morpholino sugar moiety.
89. The oligomeric compound of embodiment 88, wherein the one modified sugar moiety of the central region is a 2′-F substituted ribosyl, 2′-MOE substituted ribosyl, 2′-O-Methyl substituted ribosyl, 4′-Methyl substituted 2′-deoxyribosyl, 5′-(R)-Methyl substituted 2′-deoxyribosyl, 5′-(S)-Ethyl substituted 2′-deoxyribosyl, 5′-(R)-Ethyl substituted 2′-deoxyribosyl, 5′-(S)-Allyl substituted 2′-deoxyribosyl, 5′-(R)-Allyl substituted 2′-deoxyribosyl, 5′-stereorandom allyl substituted 2′-deoxyribosyl, unsubstituted β-L-2′-deoxyribosyl, cEt, or morpholino sugar moiety.
90. The oligomeric compound of any of embodiments 50-61 or 67-69, wherein the one modified sugar moiety of the central region is at position 1 of the central region.
91. The oligomeric compound of embodiment 90, wherein the one modified sugar moiety of the central region is a morpholino, unsubstituted β-L-2′-deoxyribosyl, or unsubstituted α-D-2′-deoxyribosyl sugar moiety.
92. The oligomeric compound of embodiment 90, wherein the one modified sugar moiety of the central region is a morpholino or unsubstituted α-D-2′-deoxyribosyl sugar moiety.
93. The oligomeric compound of any of embodiments 50-61 or 67-69, wherein the one modified sugar moiety of the central region is at position 2 of the central region.
94. The oligomeric compound of embodiment 93, wherein the one modified sugar moiety of the central region is a 2′-F substituted ribosyl, 2′-MOE substituted ribosyl, 2′-O-Methyl substituted ribosyl, 5′-(S)-Methyl substituted 2′-deoxyribosyl, 5′-Allyl substituted 2′-deoxyribosyl, unsubstituted α-D-2′-deoxyribosyl, or cEt sugar moiety.
95. The oligomeric compound of embodiment 93, wherein the one modified sugar moiety of the central region is a 2′-MOE substituted ribosyl, 2′-O-Methyl substituted ribosyl, 5′-(S)-Methyl substituted 2′-deoxyribosyl, 5′-Allyl substituted 2′-deoxyribosyl, or unsubstituted α-D-2′-deoxyribosyl sugar moiety.
96. The oligomeric compound of embodiment 94 or 95, wherein the 5′-allyl substituted 2′-deoxyribosyl sugar moiety is stereorandom at the 5′-position of the modified sugar moiety.
97. The oligomeric compound of any of embodiments 50-61 or 67-69, wherein the one modified sugar moiety of the central region is at position 3 of the central region.
98. The oligomeric compound of embodiment 97, wherein the one modified sugar moiety of the central region is a 2′-F substituted ribosyl, 2′-MOE substituted ribosyl, 2′-O-Methyl substituted ribosyl, 4′-Methyl substituted 2′-deoxyribosyl, 5′-(R)-Methyl substituted 2′-deoxyribosyl, 5′-(S)-Methyl substituted 2′-deoxyribosyl, 5′-(S)-Ethyl substituted 2′-deoxyribosyl, 5′-(R)-Ethyl substituted 2′-deoxyribosyl, 5′-(S)-Allyl substituted 2′-deoxyribosyl, 5′-(R)-Allyl substituted 2′-deoxyribosyl, 5′-stereorandom allyl substituted 2′-deoxyribosyl, unsubstituted β-L-2′-deoxyribosyl, unsubstituted β-D-2′-deoxyxylosyl, unsubstituted α-L-2′-deoxyribosyl, unsubstituted α-D-2′-deoxyribosyl, cEt, or morpholino sugar moiety.
99. The oligomeric compound of embodiment 97, wherein the one modified sugar moiety of the central region is a 2′-F substituted ribosyl, 2′-MOE substituted ribosyl, 2′-O-Methyl substituted ribosyl, 4′-Methyl substituted 2′-deoxyribosyl, 5′-(R)-Methyl substituted 2′-deoxyribosyl, 5′-(S)-Ethyl substituted 2′-deoxyribosyl, 5′-(R)-Ethyl substituted 2′-deoxyribosyl, 5′-(S)-Allyl substituted 2′-deoxyribosyl, 5′-(R)-Allyl substituted 2′-deoxyribosyl, 5′-stereorandom allyl substituted 2′-deoxyribosyl, unsubstituted β-L-2′-deoxyribosyl, unsubstituted β-D-2′-deoxyxylosyl, cEt, or morpholino sugar moiety.
100. The oligomeric compound of any of embodiments 50-61 or 67-69, wherein the one modified sugar moiety of the central region is at position 4 of the central region.
101. The oligomeric compound of embodiment 100, wherein the one modified sugar moiety of the central region is a 2′-F substituted ribosyl, 2′-MOE substituted ribosyl, 2′-O-Methyl substituted ribosyl, 4′-Methyl substituted 2′-deoxyribosyl, 5′-(R)-Methyl substituted 2′-deoxyribosyl, 5′-(S)-Methyl substituted 2′-deoxyribosyl, 5′-(S)-Ethyl substituted 2′-deoxyribosyl, 5′-(R)-Ethyl substituted 2′-deoxyribosyl, 5′-(S)-Allyl substituted 2′-deoxyribosyl, 5′-(R)-Allyl substituted 2′-deoxyribosyl, 5′-stereorandom allyl substituted 2′-deoxyribosyl, unsubstituted β-L-2′-deoxyribosyl, unsubstituted α-L-2′-deoxyribosyl, unsubstituted α-D-2′-deoxyribosyl, cEt, or morpholino sugar moiety.
102. The oligomeric compound of embodiment 100, wherein the one modified sugar moiety of the central region is a 2′-F substituted ribosyl, 2′-MOE substituted ribosyl, 2′-O-Methyl substituted ribosyl, 4′-Methyl substituted 2′-deoxyribosyl, 5′-(R)-Methyl substituted 2′-deoxyribosyl, 5′-(S)-Ethyl substituted 2′-deoxyribosyl, 5′-(R)-Ethyl substituted 2′-deoxyribosyl, 5′-(S)-Allyl substituted 2′-deoxyribosyl, 5′-(R)-Allyl substituted 2′-deoxyribosyl, 5′-stereorandom allyl substituted 2′-deoxyribosyl, unsubstituted (3-L-2′-deoxyribosyl, unsubstituted α-L-2′-deoxyribosyl, unsubstituted α-D-2′-deoxyribosyl, cEt, or morpholino sugar moiety.
103. The oligomeric compound of any of embodiments 50-61 or 67-69, wherein the one modified sugar moiety of the central region is at position 5 of the central region.
104. The oligomeric compound of embodiment 103, wherein the one modified sugar moiety of the central region is a 2′-O-Methyl substituted ribosyl or unsubstituted β-L-2′-deoxyribosyl sugar moiety.
105. The oligomeric compound of embodiment 103, wherein the one modified sugar moiety of the central region is an unsubstituted β-L-2′-deoxyribosyl sugar moiety.
106. The oligomeric compound of any of embodiments 50-61 or 67-69, wherein the one modified sugar moiety of the central region is at position 6 of the central region.
107. The oligomeric compound of embodiment 106, wherein the one modified sugar moiety of the central region is a 2′-O-Methyl substituted ribosyl, unsubstituted β-L-2′-deoxyribosyl, unsubstituted α-D-2′-deoxyribosyl, or morpholino sugar moiety.
108. The oligomeric compound of any of embodiments 50-61 or 67-69, wherein the one modified sugar moiety of the central region is at position 7 of the central region.
109. The oligomeric compound of embodiment 108, wherein the one modified sugar moiety of the central region is a 2′-O-Methyl substituted ribosyl, unsubstituted β-L-2′-deoxyribosyl, or unsubstituted α-D-2′-deoxyribosyl sugar moiety.
110. The oligomeric compound of any of embodiments 50-61 or 67-69, wherein the one modified sugar moiety of the central region is at position 8 of the central region.
111. The oligomeric compound of embodiment 110, wherein the one modified sugar moiety of the central region is a 2′-O-Methyl substituted ribosyl, unsubstituted β-L-2′-deoxyribosyl, or unsubstituted α-D-2′-deoxyribosyl sugar moiety.
112. The oligomeric compound of any of embodiments 50-61 or 67-69, wherein the one modified sugar moiety of the central region is at position 9 of the central region.
113. The oligomeric compound of embodiment 112, wherein the one modified sugar moiety of the central region is a 2′-O-Methyl substituted ribosyl or unsubstituted β-L-2′-deoxyribosyl sugar moiety.
114. The oligomeric compound of any of embodiments 50-61 or 67-69, wherein the one modified sugar moiety of the central region is at position 10 of the central region.
115. The oligomeric compound of any of embodiments 50-61 or 67-69, wherein the one modified sugar moiety of the central region is a morpholino modified sugar moiety at position 1 of the central region.
116. The oligomeric compound of any of embodiments 50-61 or 67-69, wherein the one modified sugar moiety of the central region is a modified, unsubstituted 2′-deoxyfuranosyl sugar moiety at position 1 of the central region.
117. The oligomeric compound of embodiment 116, wherein the one modified sugar moiety of the central region is an unsubstituted β-L-2′-deoxyribosyl sugar moiety at position 1 of the central region.
118. The oligomeric compound of embodiment 116, wherein the one modified sugar moiety of the central region is an unsubstituted α-D-2′-deoxyribosyl sugar moiety
119. The oligomeric compound of any of embodiments 50-61 or 67-69, wherein the one modified sugar moiety of the central region is a 2′-substituted ribosyl sugar moiety at position 2 of the central region.
120. The oligomeric compound of embodiment 119, wherein the 2′-substituted ribosyl sugar moiety at position 2 of the central region is a 2′-F ribosyl sugar moiety.
121. The oligomeric compound of embodiment 119, wherein the 2′-substituted ribosyl sugar moiety at position 2 of the central region is a 2′-MOE ribosyl sugar moiety.
122. The oligomeric compound of embodiment 119, wherein the 2′-substituted ribosyl sugar moiety at position 2 of the central region is a 2′-O-methyl ribosyl sugar moiety.
123. The oligomeric compound of any of embodiments 50-61 or 67-69, wherein the one modified sugar moiety of the central region is a 5′-alkyl substituted 2′-deoxyribosyl sugar moiety at position 2 of the central region.
124. The oligomeric compound of embodiment 123, wherein the 5′-alkyl substituted 2′-deoxyribosyl sugar moiety at position 2 of the central region is a 5′-(S)Me 2′-deoxyribosyl sugar moiety.
125. The oligomeric compound of embodiment 123, wherein the 5′-alkyl substituted 2′-deoxyribosyl sugar moiety at position 2 of the central region is a 5′-allyl 2′-deoxyribosyl sugar moiety.
126. The oligomeric compound of embodiment 125, wherein the 5′-allyl 2′-deoxyribosyl sugar moiety is stereorandom at the 5′-position of the 5′-allyl 2′-deoxyribosyl sugar moiety.
127. The oligomeric compound of any of embodiments 50-61 or 67-69, wherein the one modified sugar moiety of the central region is a modified, unsubstituted 2′-deoxyfuranosyl sugar moiety at position 2 of the central region.
128. The oligomeric compound of embodiment 127, wherein the modified, unsubstituted 2′-deoxyfuranosyl sugar moiety at position 2 of the central region is a α-D-2′-deoxyribosyl modified sugar moiety.
129. The oligomeric compound of any of embodiments 50-61 or 67-69, wherein the one modified sugar moiety of the central region is a cEt or LNA sugar moiety at position 2 of the central region.
130. The oligomeric compound of any of embodiments 50-61 or 67-69, wherein the one modified sugar moiety of the central region is a 2′-substituted ribosyl sugar moiety at position 3 of the central region.
131. The oligomeric compound of embodiment 130, wherein the 2′-substituted ribosyl sugar moiety at position 3 of the central region is a 2′-F ribosyl sugar moiety.
132. The oligomeric compound of embodiment 130, wherein the 2′-substituted ribosyl sugar moiety at position 3 of the central region is a 2′-MOE ribosyl sugar moiety.
133. The oligomeric compound of embodiment 130, wherein the 2′-substituted ribosyl sugar moiety at position 3 of the central region is a 2′-O-methyl ribosyl sugar moiety.
134. The oligomeric compound of any of embodiments 50-61 or 67-69, wherein the one modified sugar moiety of the central region is a 4′-alkyl substituted 2′-deoxyribosyl sugar moiety at position 3 of the central region.
135. The oligomeric compound of embodiment 134, wherein the 4′-substituted 2′-deoxyribosyl sugar moiety at position 3 of the central region is a 4′-methyl 2′-deoxyribosyl sugar moiety.
136. The oligomeric compound of any of embodiments 50-61 or 67-69, wherein the one modified sugar moiety of the central region is a 5′-alkyl substituted 2′-deoxyribosyl sugar moiety at position 3 of the central region.
137. The oligomeric compound of embodiment 136, wherein the 5′-alkyl substituted 2′-deoxyribosyl sugar moiety at position 3 of the central region is a 5′-(R)-methyl 2′-deoxyribosyl sugar moiety.
138. The oligomeric compound of embodiment 136, wherein the 5′-alkyl substituted 2′-deoxyribosyl sugar moiety at position 3 of the central region is a 5′-(S)-methyl 2′-deoxyribosyl sugar moiety.
139. The oligomeric compound of embodiment 136, wherein the 5′-alkyl substituted 2′-deoxyribosyl sugar moiety at position 3 of the central region is a 5′-stereorandom methyl 2′-deoxyribosyl sugar moiety.
140. The oligomeric compound of embodiment 136, wherein the 5′-alkyl substituted 2′-deoxyribosyl sugar moiety at position 3 of the central region is a 5′-(R)-ethyl 2′-deoxyribosyl sugar moiety.
141. The oligomeric compound of embodiment 136, wherein the 5′-alkyl substituted 2′-deoxyribosyl sugar moiety at position 3 of the central region is a 5′-(S)-ethyl 2′-deoxyribosyl sugar moiety.
142. The oligomeric compound of embodiment 136, wherein the 5′-alkyl substituted 2′-deoxyribosyl sugar moiety at position 3 of the central region is a 5′-stereorandom ethyl 2′-deoxyribosyl sugar moiety.
143. The oligomeric compound of embodiment 136, wherein the 5′-alkyl substituted 2′-deoxyribosyl sugar moiety at position 3 of the central region is a 5′-(R)-allyl 2′-deoxyribosyl sugar moiety.
144. The oligomeric compound of embodiment 136, wherein the 5′-alkyl substituted 2′-deoxyribosyl sugar moiety at position 3 of the central region is a 5′-(S)-allyl 2′-deoxyribosyl sugar moiety.
145. The oligomeric compound of embodiment 136, wherein the 5′-alkyl substituted 2′-deoxyribosyl sugar moiety at position 3 of the central region is a 5′-stereorandom allyl 2′-deoxyribosyl sugar moiety.
146. The oligomeric compound of any of embodiments 50-61 or 67-69, wherein the one modified sugar moiety of the central region is a modified, unsubstituted 2′-deoxyfuranosyl sugar moiety at position 3 of the central region.
147. The oligomeric compound of embodiment 146, wherein the modified, unsubstituted 2′-deoxyfuranosyl sugar moiety at position 3 of the central region is an α-D-2′-deoxyribosyl modified sugar moiety.
148. The oligomeric compound of embodiment 146, wherein the modified, unsubstituted 2′-deoxyfuranosyl sugar moiety at position 3 of the central region is an α-L-2′-deoxyribosyl modified sugar moiety.
149. The oligomeric compound of embodiment 146, wherein the modified, unsubstituted 2′-deoxyfuranosyl sugar moiety at position 3 of the central region is an β-L-2′-deoxyribosyl modified sugar moiety.
150. The oligomeric compound of embodiment 146, wherein the modified, unsubstituted 2′-deoxyfuranosyl sugar moiety at position 3 of the central region is an β-D-2′-deoxyxylosyl modified sugar moiety.
151. The oligomeric compound of any of embodiments 50-61 or 67-69, wherein the one modified sugar moiety of the central region is a bicyclic ribosyl sugar moiety at position 3 of the central region.
152. The oligomeric compound of embodiment 151, wherein the bicyclic ribosyl sugar moiety at position 3 of the central region is a cEt or LNA sugar moiety.
153. The oligomeric compound of embodiment 151, wherein the bicyclic ribosyl sugar moiety at position 3 of the central region is a cEt sugar moiety.
154. The oligomeric compound of embodiment 151, wherein the bicyclic ribosyl sugar moiety at position 3 of the central region is a LNA sugar moiety.
155. The oligomeric compound of any of embodiments 50-61 or 67-69, wherein the one modified sugar moiety of the central region is a morpholino sugar moiety at position 3 of the central region.
156. The oligomeric compound of any of embodiments 50-61 or 67-69, wherein the one modified sugar moiety of the central region is a 2′-substituted ribosyl sugar moiety at position 4 of the central region.
157. The oligomeric compound of embodiment 156, wherein the 2′-substituted ribosyl sugar moiety at position 4 of the central region is a 2′-F ribosyl sugar moiety.
158. The oligomeric compound of embodiment 156, wherein the 2′-substituted ribosyl sugar moiety at position 4 of the central region is a 2′-MOE ribosyl sugar moiety.
159. The oligomeric compound of embodiment 156, wherein the 2′-substituted ribosyl sugar moiety at position 4 of the central region is a 2′-O-methyl ribosyl sugar moiety.
160. The oligomeric compound of any of embodiments 50-61 or 67-69, wherein the one modified sugar moiety of the central region is a 4′-alkyl substituted 2′-deoxyribosyl sugar moiety at position 4 of the central region.
161. The oligomeric compound of embodiment 160, wherein the 4′-substituted 2′-deoxyribosyl sugar moiety at position 4 of the central region is a 4′-methyl 2′-deoxyribosyl sugar moiety.
162. The oligomeric compound of any of embodiments 50-61 or 67-69, wherein the one modified sugar moiety of the central region is a 5′-alkyl substituted 2′-deoxyribosyl sugar moiety at position 4 of the central region.
163. The oligomeric compound of embodiment 162, wherein the 5′-alkyl substituted 2′-deoxyribosyl sugar moiety at position 4 of the central region is a 5′-(R)-methyl 2′-deoxyribosyl sugar moiety.
164. The oligomeric compound of embodiment 162, wherein the 5′-alkyl substituted 2′-deoxyribosyl sugar moiety at position 4 of the central region is a 5′-(S)-methyl 2′-deoxyribosyl sugar moiety.
165. The oligomeric compound of embodiment 162, wherein the 5′-alkyl substituted 2′-deoxyribosyl sugar moiety at position 4 of the central region is a 5′-stereorandom methyl 2′-deoxyribosyl sugar moiety.
166. The oligomeric compound of embodiment 162, wherein the 5′-alkyl substituted 2′-deoxyribosyl sugar moiety at position 4 of the central region is a 5′-(R)-ethyl 2′-deoxyribosyl sugar moiety.
167. The oligomeric compound of embodiment 162, wherein the 5′-alkyl substituted 2′-deoxyribosyl sugar moiety at position 4 of the central region is a 5′-(S)-ethyl 2′-deoxyribosyl sugar moiety.
168. The oligomeric compound of embodiment 162, wherein the 5′-alkyl substituted 2′-deoxyribosyl sugar moiety at position 4 of the central region is a 5′-stereorandom ethyl 2′-deoxyribosyl sugar moiety.
169. The oligomeric compound of embodiment 162, wherein the 5′-alkyl substituted 2′-deoxyribosyl sugar moiety at position 4 of the central region is a 5′-(R)-allyl 2′-deoxyribosyl sugar moiety.
170. The oligomeric compound of embodiment 162, wherein the 5′-alkyl substituted 2′-deoxyribosyl sugar moiety at position 4 of the central region is a 5′-(S)-allyl 2′-deoxyribosyl sugar moiety.
171. The oligomeric compound of embodiment 162, wherein the 5′-alkyl substituted 2′-deoxyribosyl sugar moiety at position 4 of the central region is a 5′-stereorandom allyl 2′-deoxyribosyl sugar moiety.
172. The oligomeric compound of any of embodiments 50-61 or 67-69, wherein the one modified sugar moiety of the central region is a modified, unsubstituted 2′-deoxyfuranosyl sugar moiety at position 4 of the central region.
173. The oligomeric compound of embodiment 172, wherein the modified, unsubstituted 2′-deoxyfuranosyl sugar moiety at position 4 of the central region is an α-D-2′-deoxyribosyl modified sugar moiety.
174. The oligomeric compound of embodiment 172, wherein the modified, unsubstituted 2′-deoxyfuranosyl sugar moiety at position 4 of the central region is an α-L-2′-deoxyribosyl modified sugar moiety.
175. The oligomeric compound of embodiment 172, wherein the modified, unsubstituted 2′-deoxyfuranosyl sugar moiety at position 4 of the central region is an β-L-2′-deoxyribosyl modified sugar moiety.
176. The oligomeric compound of any of embodiments 50-61 or 67-69, wherein the one modified sugar moiety of the central region is a bicyclic ribosyl sugar moiety at position 4 of the central region.
177. The oligomeric compound of embodiment 176, wherein the bicyclic ribosyl sugar moiety at position 4 of the central region is a cEt or LNA sugar moiety.
178. The oligomeric compound of embodiment 176, wherein the bicyclic ribosyl sugar moiety at position 4 of the central region is a cEt sugar moiety.
179. The oligomeric compound of embodiment 176, wherein the bicyclic ribosyl sugar moiety at position 4 of the central region is a LNA sugar moiety.
180. The oligomeric compound of any of embodiments 50-61 or 67-69, wherein the one modified sugar moiety of the central region is a morpholino sugar moiety at position 4 of the central region.
181. The oligomeric compound of any of embodiments 50-61 or 67-69, wherein the one modified sugar moiety of the central region is a 2′-O-methyl substituted ribosyl sugar moiety at position 5 of the central region.
182. The oligomeric compound of any of embodiments 50-61 or 67-69, wherein the one modified sugar moiety of the central region is a modified, unsubstituted 2′-deoxyfuranosyl sugar moiety at position 5 of the central region.
183. The oligomeric compound of embodiment 182, wherein the modified, unsubstituted 2′-deoxyfuranosyl sugar moiety at position 5 of the central region is a β-L-2′-deoxyribosyl sugar moiety.
184. The oligomeric compound of any of embodiments 50-61 or 67-69, wherein the one modified sugar moiety of the central region is a morpholino sugar moiety at position 6 of the central region.
185. The oligomeric compound of any of embodiments 50-61 or 67-69, wherein the one modified sugar moiety of the central region is a 2′-O-methyl substituted ribosyl sugar moiety at position 6 of the central region.
186. The oligomeric compound of any of embodiments 50-61 or 67-69, wherein the one modified sugar moiety of the central region is a modified, unsubstituted 2′-deoxyfuranosyl sugar moiety at position 6 of the central region.
187. The oligomeric compound of embodiment 186, wherein the modified, unsubstituted 2′-deoxyfuranosyl sugar moiety at position 6 of the central region is a β-L-2′-deoxyribosyl sugar moiety.
188. The oligomeric compound of embodiment 186, wherein the modified, unsubstituted 2′-deoxyfuranosyl sugar moiety at position 6 of the central region is an α-D-2′-deoxyribosyl sugar moiety.
189. The oligomeric compound of any of embodiments 50-61 or 67-69, wherein the one modified sugar moiety of the central region is a 2′-O-methyl substituted ribosyl sugar moiety at position 7 of the central region.
190. The oligomeric compound of any of embodiments 50-61 or 67-69, wherein the one modified sugar moiety of the central region is a modified, unsubstituted 2′-deoxyfuranosyl sugar moiety at position 7 of the central region.
191. The oligomeric compound of embodiment 190, wherein the modified, unsubstituted 2′-deoxyfuranosyl sugar moiety at position 7 of the central region is a β-L-2′-deoxyribosyl sugar moiety.
192. The oligomeric compound of embodiment 190, wherein the modified, unsubstituted 2′-deoxyfuranosyl sugar moiety at position 7 of the central region is an α-D-2′-deoxyribosyl sugar moiety.
193. The oligomeric compound of any of embodiments 50-61 or 67-69, wherein the one modified sugar moiety of the central region is a 2′-O-methyl substituted ribosyl sugar moiety at position 8 of the central region.
194. The oligomeric compound of any of embodiments 50-61 or 67-69, wherein the one modified sugar moiety of the central region is a modified, unsubstituted 2′-deoxyfuranosyl sugar moiety at position 8 of the central region.
195. The oligomeric compound of embodiment 194, wherein the modified, unsubstituted 2′-deoxyfuranosyl sugar moiety at position 8 of the central region is a β-L-2′-deoxyribosyl sugar moiety.
196. The oligomeric compound of embodiment 194, wherein the modified, unsubstituted 2′-deoxyfuranosyl sugar moiety at position 8 of the central region is an α-D-2′-deoxyribosyl sugar moiety.
197. The oligomeric compound of any of embodiments 50-61 or 67-69, wherein the one modified sugar moiety of the central region is a 2′-O-methyl substituted ribosyl sugar moiety at position 9 of the central region.
198. The oligomeric compound of any of embodiments 50-61 or 67-69, wherein the one modified sugar moiety of the central region is a modified, unsubstituted 2′-deoxyfuranosyl sugar moiety at position 9 of the central region.
199. The oligomeric compound of embodiment 198, wherein the modified, unsubstituted 2′-deoxyfuranosyl sugar moiety at position 9 of the central region is a β-L-2′-deoxyribosyl sugar moiety.
200. The oligomeric compound of any of embodiments 50-61 or 67-69, wherein the one modified sugar moiety of the central region is a 2′-O-methyl substituted ribosyl sugar moiety at position 2, 3, 4, 5, 6, 7, 8, or 9 of the central region.
201. The oligomeric compound of any of embodiments 50-61 or 67-69, wherein the one modified sugar moiety of the central region is a 2′-O-methyl substituted ribosyl sugar moiety at position 2, 3, 4, 8, or 9 of the central region.
202. The oligomeric compound of any of embodiments 50-61 or 67-69, wherein the one modified sugar moiety of the central region is a 2′-F substituted ribosyl sugar moiety at position 2, 3, or 4 of the central region.
203. The oligomeric compound of any of embodiments 50-61 or 67-69, wherein the one modified sugar moiety of the central region is a 2′-F substituted ribosyl sugar moiety at position 3 or 4 of the central region.
204. The oligomeric compound of any of embodiments 50-61 or 67-69, wherein the one modified sugar moiety of the central region is a 2′-MOE substituted ribosyl sugar moiety at position 2, 3, or 4 of the central region.
205. The oligomeric compound of any of embodiments 50-61 or 67-69, wherein the one modified sugar moiety of the central region is a 4′-methyl substituted 2′-deoxyribosyl sugar moiety at position 3 or 4 of the central region.
206. The oligomeric compound of any of embodiments 50-61 or 67-69, wherein the one modified sugar moiety of the central region is a 5′-(R)-methyl substituted 2′-deoxyribosyl sugar moiety at position 3 or 4 of the central region.
207. The oligomeric compound of any of embodiments 50-61 or 67-69, wherein the one modified sugar moiety of the central region is a 5′-(S)-methyl substituted 2′-deoxyribosyl sugar moiety at position 2, 3, or 4 of the central region.
208. The oligomeric compound of any of embodiments 50-61 or 67-69, wherein the one modified sugar moiety of the central region is a 5′-(R)-ethyl substituted 2′-deoxyribosyl sugar moiety at position 3 or 4 of the central region.
209. The oligomeric compound of any of embodiments 50-61 or 67-69, wherein the one modified sugar moiety of the central region is a 5′-(S)-ethyl substituted 2′-deoxyribosyl sugar moiety at position 3 or 4 of the central region.
210. The oligomeric compound of any of embodiments 50-61 or 67-69, wherein the one modified sugar moiety of the central region is a 5′-(R)-allyl substituted 2′-deoxyribosyl sugar moiety at position 3 or 4 of the central region.
211. The oligomeric compound of any of embodiments 50-61 or 67-69, wherein the one modified sugar moiety of the central region is a 5′-(S)-allyl substituted 2′-deoxyribosyl sugar moiety at position 3 or 4 of the central region.
212. The oligomeric compound of any of embodiments 50-61 or 67-69, wherein the one modified sugar moiety of the central region is a 5′-stereorandom allyl substituted 2′-deoxyribosyl sugar moiety at position 2, 3, or 4 of the central region.
213. The oligomeric compound of any of embodiments 50-61 or 67-69, wherein the one modified sugar moiety of the central region is a cEt ribosyl sugar moiety at position 2, 3, or 4 of the central region.
214. The oligomeric compound of any of embodiments 50-61 or 67-69, wherein the one modified sugar moiety of the central region is a cEt ribosyl sugar moiety at position 3 or 4 of the central region.
215. The oligomeric compound of any of embodiments 50-61 or 67-69, wherein the one modified sugar moiety of the central region is a LNA ribosyl sugar moiety at position 2, 3, or 4 of the central region.
216. The oligomeric compound of any of embodiments 50-61 or 67-69, wherein the one modified sugar moiety of the central region is a LNA ribosyl sugar moiety at position 3 or 4 of the central region.
217. The oligomeric compound of any of embodiments 50-61 or 67-69, wherein the one modified sugar moiety of the central region is a morpholino sugar moiety at position 1, 3, 4, or 6 of the central region.
218. The oligomeric compound of any of embodiments 50-61 or 67-69, wherein the one modified sugar moiety of the central region is a morpholino sugar moiety at position 1, 3, or 4 of the central region.
219. The oligomeric compound of any of embodiments 50-61 or 67-69, wherein the one modified sugar moiety of the central region is a modified, unsubstituted 2′-deoxyfuranosyl sugar moiety.
220. The oligomeric compound of any of embodiments 50-61 or 67-69, wherein the one modified sugar moiety of the central region is a modified, unsubstituted 2′-deoxyfuranosyl sugar moiety at position 1, 2, 3, 4, 5, 6, 7, 8, or 9 of the central region.
221. The oligomeric compound of any of embodiments 50-61 or 67-69, wherein the one modified sugar moiety of the central region is a modified, unsubstituted 2′-deoxyfuranosyl sugar moiety at position 1, 2, 3, 4, 5, or 9 of the central region.
222. The oligomeric compound of any of embodiments 50-61 or 67-69, wherein the one modified sugar moiety of the central region is an unsubstituted α-D-2′-deoxyribosyl sugar moiety.
223. The oligomeric compound of any of embodiments 50-61 or 67-69, wherein the one modified sugar moiety of the central region is an unsubstituted α-D-2′-deoxyribosyl sugar moiety at position 1, 2, 3, 4, 6, or 8 of the central region.
224. The oligomeric compound of any of embodiments 50-61 or 67-69, wherein the one modified sugar moiety of the central region is an unsubstituted α-D-2′-deoxyribosyl sugar moiety at position 1, 2, or 4 of the central region.
225. The oligomeric compound of any of embodiments 50-61 or 67-69, wherein the one modified sugar moiety of the central region is an unsubstituted α-L-2′-deoxyribosyl sugar moiety.
226. The oligomeric compound of any of embodiments 50-61 or 67-69, wherein the one modified sugar moiety of the central region is an unsubstituted α-L-2′-deoxyribosyl sugar moiety at position 3, 4, or 7 of the central region.
227. The oligomeric compound of any of embodiments 50-61 or 67-69, wherein the one modified sugar moiety of the central region is an unsubstituted β-L-2′-deoxyribosyl sugar moiety.
228. The oligomeric compound of any of embodiments 50-61 or 67-69, wherein the one modified sugar moiety of the central region is an unsubstituted β-L-2′-deoxyribosyl sugar moiety at position 1, 3, 4, 5, 6, 7, 8, or 9 of the central region.
229. The oligomeric compound of any of embodiments 50-61 or 67-69, wherein the one modified sugar moiety of the central region is an unsubstituted β-L-2′-deoxyribosyl sugar moiety at position 3, 4, 5, or 9 of the central region.
230. The oligomeric compound of any of embodiments 50-61 or 67-69, wherein the one modified sugar moiety of the central region is an unsubstituted β-D-2′-deoxyxylosyl sugar moiety.
231. The oligomeric compound of any of embodiments 53-55 or 60-230, wherein the one modified internucleoside linkage of the central region other than phosphorothioate is a phosphonate or phosphotriester internucleoside linkage.
232. The oligomeric compound of embodiment 231, wherein the one modified internucleoside linkage of the central region other than phosphorothioate is an alkyl phosphonate or alkoxy phosphonate internucleoside linkage.
233. The oligomeric compound of embodiment 232, wherein the one modified internucleoside linkage of the central region other than phosphorothioate is a methoxypropyl internucleoside linkage.
234. The oligomeric compound of embodiment 232, wherein the one modified internucleoside linkage of the central region other than phosphorothioate is a methyl phosphonate internucleoside linkage.
235. The oligomeric compound of embodiment 232, wherein the one modified internucleoside linkage of the central region other than phosphorothioate is an isopropyl phosphonate internucleoside linkage.
236. The oligomeric compound of embodiment 232, wherein the one modified internucleoside linkage of the central region other than phosphorothioate is an isobutyl phosphonate internucleoside linkage.
237. The oligomeric compound of embodiment 231, wherein the one modified internucleoside linkage of the central region other than phosphorothioate is a phosphonoacetate internucleoside linkage.
238. The oligomeric compound of embodiment 231, wherein the one modified internucleoside linkage of the central region other than phosphorothioate is an isopropyl phosphotriester internucleoside linkage.
239. The oligomeric compound of embodiment 231, wherein the one modified internucleoside linkage of the central region other than phosphorothioate is a tetrahydropyran phosphotriester internucleoside linkage.
240. The oligomeric compound of any of embodiments 53, 55, or 60-230, wherein the one modified internucleoside linkage of the central region other than phosphorothioate is a formacetal internucleoside linkage.
241. The oligomeric compound of any of embodiments 53, 55, or 60-230, wherein the one modified internucleoside linkage of the central region other than phosphorothioate is an acetamide internucleoside linkage.
242. The oligomeric compound of any of embodiments 53, 55, or 60-230, wherein the one modified internucleoside linkage of the central region other than phosphorothioate is a thioacetamide internucleoside linkage.
243. The oligomeric compound of any of embodiments 231-242, wherein the one modified internucleoside linakge of the central region other than phosphorothioate is between the nucleosides at positions 1 and 2 of the central region.
244. The oligomeric compound of any of embodiments 231-242, wherein the one modified internucleoside linkage of the central region other than phosphorothioate is between the nucleosides at positions 2 and 3 of the central region.
245. The oligomeric compound of any of embodiments 231-242, wherein the one modified internucleoside linkage of the central region other than phosphorothioate is between the nucleosides at positions 3 and 4 of the central region.
246. The oligomeric compound of any of embodiments 231-242, wherein the one modified internucleoside linkage of the central region other than phosphorothioate is between the nucleosides at positions 4 and 5 of the central region.
247. The oligomeric compound of any of embodiments 231-246, wherein the one modified internucleoside linkage of the central region other than phosphorothioate is directly linked to a nucleoside comprising a modified sugar moiety.
248. The oligomeric compound of any of embodiments 56-230, wherein the two neutral internucleoside linkages of the central region are independently selected from a phosphonate internucleoside linkage, phosphotriester internucleoside linkage, and a neutral internucleoside linkage that does not contain phosphorus.
249. The oligomeric compound of embodiment 248, wherein at least one of the modified internucleoside linkages of the central region other than phosphorothioate is an alkyl phosphonate or alkoxy phosphonate internucleoside linkage.
250. The oligomeric compound of embodiment 248, wherein at least one of the modified internucleoside linkages of the central region other than phosphorothioate is a methoxypropyl internucleoside linkage.
251. The oligomeric compound of embodiment 248, wherein at least one of the modified internucleoside linkages of the central region other than phosphorothioate is a methyl phosphonate internucleoside linkage.
252. The oligomeric compound of embodiment 248, wherein at least one of the modified internucleoside linkages of the central region other than phosphorothioate is an isopropyl phosphonate internucleoside linkage.
253. The oligomeric compound of embodiment 248, wherein at least one of the modified internucleoside linkages of the central region other than phosphorothioate is an isobutyl phosphonate internucleoside linkage.
254. The oligomeric compound of embodiment 248, wherein at least one of the modified internucleoside linkages of the central region other than phosphorothioate is a phosphonoacetate internucleoside linkage.
255. The oligomeric compound of embodiment 248, wherein at least one of the modified internucleoside linkages of the central region other than phosphorothioate is an isopropyl phosphotriester internucleoside linkage.
256. The oligomeric compound of embodiment 248, wherein at least one of the modified internucleoside linkages of the central region other than phosphorothioate is a tetrahydropyran phosphotriester internucleoside linkage.
257. The oligomeric compound of embodiment 248, wherein at least one of the modified internucleoside linkages of the central region other than phosphorothioate is a formacetal internucleoside linkage.
258. The oligomeric compound of embodiment 248, wherein at least one of the modified internucleoside linkages of the central region other than phosphorothioate is an acetamide internucleoside linkage.
259. The oligomeric compound of embodiment 248, wherein at least one of the modified internucleoside linkages of the central region other than phosphorothioate is a thioacetamide internucleoside linkage.
260. The oligomeric compound of any of embodiments 248-259, wherein the two modified internucleoside linkages other than phosphorothiaote of the central region are adjacent to each other.
261. The oligomeric compound of any of clamis 248-260, wherein the two modified internucleoside linkages other than phosphorothioate of the central region are the same as one another.
262. The oligomeric compound of any of embodiments 248-261, wherein one of the modified internucleoside linkages of the central region other than phosphorothioate is between the nucleosides at positions 1 and 2 of the central region.
263. The oligomeric compound of any of embodiments 248-261, wherein one of the modified internucleoside linkages of the central region other than phosphorothioate is between the nucleosides at positions 2 and 3 of the central region.
264. The oligomeric compound of any of embodiments 248-261, wherein one of the modified internucleoside linkages of the central region other than phosphorothioate is between the nucleosides at positions 3 and 4 of the central region.
265. The oligomeric compound of any of embodiments 248-261, wherein one of the modified internucleoside linkages of the central region other than phosphorothioate is between the nucleosides at positions 4 and 5 of the central region.
266. The oligomeric compound of any of embodiments 248-261, wherein one of the modified internucleoside linkages of the central region other than phosphorothioate is directly linked to a nucleoside comprising a modified sugar moiety.
267. The oligomeric compound of any of embodiments 60-266, wherein the one modified nucleobase other than 5-methylcytosine of the central region is 2-thiothymine, 6-methyladenine, inosine, or pseudouracil.
268. The oligomeric compound of embodiment 267, wherein the one modified nucleobase other than 5-methylcytosine is at position 1, 2, 3, or 4 of the central region.
269. The oligomeric compound of embodiment 267, wherein the one modified nucleobase other than 5-methylcytosine is at position 2, 3, or 4 of the central region.
270. The oligomeric compound of embodiment 267, wherein the one modified nucleobase other than 5-methylcytosine is at position 1 of the central region.
271. The oligomeric compound of embodiment 267, wherein the one modified nucleobase other than 5-methylcytosine is at position 2 of the central region.
272. The oligomeric compound of embodiment 267, wherein the one modified nucleobase other than 5-methylcytosine is at position 3 of the central region.
273. The oligomeric compound of embodiment 267, wherein the one modified nucleobase other than 5-methylcytosine is at position 4 of the central region.
274. The oligomeric compound of any of embodiments 267 or 270, wherein the one modified nucleobase other than 5-methylcytosine is 2-thiothymine.
275. The oligomeric compound of any of embodiments 267 or 271, wherein the one modified nucleobase other than 5-methylcytosine is 6-methyladenine.
276. The oligomeric compound of any of embodiments 267 or 271, wherein the one modified nucleobase other than 5-methylcytosine is inosine.
277. The oligomeric compound of any of embodiments 267-273, wherein the one modified nucleobase other than 5-methylcytosine is pseudouracil.
278. The oligomeric compound of embodiment 277, wherein the nucleoside comprising the pseudouracil nucleobase comprises an unmodified ribosyl sugar moiety.
279. The oligomeric compound of any of embodiments 1-52, 60-230, or 267-278, wherein each internucleoside linkage of the central region is independently selected from among a phosphodiester or a phosphorothioate internucleoside linkage.
280. The oligomeric compound of embodiment 279, wherein each internucleoside of the central region is a phosphorothioate internucleoside linkage.
281. The oligomeric compound of any of embodiments 1-279, wherein the central region does not comprise any phosphodiester internucleoside linkages.
282. The oligomeric compound of any of embodiments 1-281, wherein each phosphorothioate internucleoside linkage of the oligomeric compound is strereorandom.
283. The oligomeric compound of any of embodiments 1-281, wherein at least one phosphorothioate internucleoside linkage of the oligomeric compound is in the Rp configuration.
284. The oligomeric compound of any of embodiments 1-281, wherein at least one phosphorothioate internucleoside linkage of the oligomeric compound is in the Sp configuration.
285. The oligomeric compound of any of embodiments 1-284, wherein the nucleobase sequence of the modified oligonucleotide is complementary to a target RNA.
286. The oligomeric compound of embodiment 285, wherein the nucleobase sequence of the modified oligonucleotide is at least 75% complementary to the target RNA.
287. The oligomeric compound of embodiment 285, wherein the nucleobase sequence of the modified oligonucleotide is at least 80% complementary to the target RNA.
288. The oligomeric compound of embodiment 285, wherein the nucleobase sequence of the modified oligonucleotide is at least 85% complementary to the target RNA.
289. The oligomeric compound of embodiment 285, wherein the nucleobase sequence of the modified oligonucleotide is at least 90% complementary to the target RNA.
290. The oligomeric compound of embodiment 285, wherein the nucleobase sequence of the modified oligonucleotide is at least 95% complementary to the target RNA.
291. The oligomeric compound of embodiment 285, wherein the nucleobase sequence of the modified oligonucleotide is 100% complementary to the target RNA.
292. The oligomeric compound of any of embodiments 285-291, wherein the target RNA is a target mRNA or a target pre-mRNA.
293. The oligomeric compound of embodiment 292, wherein the target mRNA or target pre-mRNA is not a mouse, rat, monkey, or human PTEN RNA.
294. The oligomeric compound of embodiment 292 or 293, wherein the target mRNA or target pre-mRNA is not a mouse, rat, monkey, or human SRB-1 RNA.
295. The oligomeric compound of any of embodiments 292-294, wherein the target mRNA or target pre-mRNA is not a mouse, rat, monkey, or human MYD88 RNA.
296. The oligomeric compound of any of embodiments 292-295, wherein the target mRNA or target pre-mRNA is not a mouse, rat, monkey, or human HTT RNA, is not a mouse, rat, monkey, or human SOD1 RNA, and is not a mouse, rat, monkey, or human alpha-synuclein RNA.
297. The oligomeric compound of any of embodiments 285-296, wherein the target RNA is a human RNA.
298. The oligomeric compound of any of embodiments 285-297, wherein the target RNA is expressed in the liver.
299. The oligomeric compound of any of embodiments 285-298, wherein the target RNA is a liver target.
300. The oligomeric compound of any of embodiments 285-297, wherein the target RNA is not expressed in the liver.
301. The oligomeric compound of any of embodiments 285-298 or 300, wherein the target RNA is not a liver target.
302. The oligomeric compound of any of embodiments 285-299, wherein modulation of the expression of the target RNA in the liver is associated with treating a disorder or condition.
303. The oligomeric compound of embodiment 302, wherein the disorder or condition is a liver disorder or condition.
304. The oligomeric compound of any of embodiments 285-303, wherein the target RNA is expressed in the central nervous system.
305. The oligomeric compound of any of embodiments 285-303, wherein the target RNA is not expressed in the central nervous system.
306. The oligomeric compound of any of embodiments 285-298, 300, 301, or 304, wherein the target RNA is a central nervous system target.
307. The oligomeric compound of any of embodiments 285-305, wherein the target RNA is not a central nervous system target.
308. The oligomeric compound of any of embodiments 285-298, 300-301, 304, or 306, wherein the target RNA is a MeCP2, DUX4, HDAC2, APP, Ataxin 1, Ataxin 2, Ataxin 3, Ataxin 6, Ataxin 7, C9ORF72, UBE3A, Prion, PMP22, Tau, LRRK2, LINGO2, GYS1, KCNT1, IRF8, Progranulin, or GFAP RNA.
309. The oligomeric compound of any of embodiments 285-297, 300-301, 304, or 306, wherein the target RNA is a HTT RNA.
310. The oligomeric compound of embodiment 308, wherein the target RNA is a MeCP2 RNA.
311. The oligomeric compound of embodiment 308, wherein the target RNA is a DUX4 RNA.
312. The oligomeric compound of embodiment 308, wherein the target RNA is a HDAC2 RNA.
313. The oligomeric compound of embodiment 308, wherein the target RNA is a Ataxin 1 RNA.
314. The oligomeric compound of embodiment 308, wherein the target RNA is a Ataxin 2 RNA.
315. The oligomeric compound of embodiment 308, wherein the target RNA is a Ataxin 3 RNA.
316. The oligomeric compound of embodiment 308, wherein the target RNA is a Ataxin 6 RNA.
317. The oligomeric compound of embodiment 308, wherein the target RNA is a Ataxin 7 RNA.
318. The oligomeric compound of embodiment 308, wherein the target RNA is a C9ORF72 RNA.
319. The oligomeric compound of embodiment 285-297, 300-301, 304, or 306, wherein the target RNA is an alpha-synuclein RNA.
320. The oligomeric compound of embodiment 308, wherein the target RNA is an UBE3A RNA.
321. The oligomeric compound of embodiment 285-297, 300-301, 304, or 306, wherein the target RNA is a SOD1 RNA.
322. The oligomeric compound of embodiment 308, wherein the target RNA is a Prion RNA.
323. The oligomeric compound of embodiment 308, wherein the target RNA is a PMP22 RNA.
324. The oligomeric compound of embodiment 308, wherein the target RNA is a Tau RNA.
325. The oligomeric compound of embodiment 308, wherein the target RNA is a LRRK2 RNA.
326. The oligomeric compound of embodiment 308, wherein the target RNA is an APP RNA.
327. The oligomeric compound of embodiment 308, wherein the target RNA is a LINGO2 RNA.
328. The oligomeric compound of embodiment 308, wherein the target RNA is a GYS1 RNA.
329. The oligomeric compound of embodiment 308, wherein the target RNA is a KCNT1 RNA.
330. The oligomeric compound of embodiment 308, wherein the target RNA is a IRF8 RNA.
331. The oligomeric compound of embodiment 308, wherein the target RNA is a Progranulin RNA.
332. The oligomeric compound of embodiment 308, wherein the target RNA is a GFAP RNA.
333. The oligomeric compound of any of embodiments 304, 306, or 308-332, wherein modulation of the expression of the target RNA in the central nervous system is associated with treating a disorder or condition.
334. The oligomeric compound of embodiment 333, wherein the disorder or condition is a neurological disorder or condition.
335. The oligomeric compound of embodiment 333 or 334, wherein the disorder or condition alters the function of sensory or motor neurons.
336. The oligomeric compound of any of embodiments 333-335, wherein the disorder or condition alters the function of sensory neurons.
337. The oligomeric compound of any of embodiments 333-336, wherein the disorder or condition alters the function of motor neurons.
338. The oligomeric compound of any of embodiments 333-337, wherein the disorder or condition alters the function of glial cells.
339. The oligomeric compound of embodiment 338, wherein the disorder or condition alters the function of astrocytes.
340. The oligomeric compound of embodiment 338, wherein the disorder or condition alters the function of oligodendrocytes.
341. The oligomeric compound of embodiment 338, wherein the disorder or condition alters the function of microglia.
342. The oligomeric compound of embodiment 338, wherein the disorder or condition alters the function of ependymal cells.
343. The oligomeric compound of any of embodiments 333-342, wherein the disorder or condition is Alzheimer's Disease, Amyotrophic Lateral Sclerosis, Parkinson's Disease, a Spinocerebellar ataxia, Angelman Syndrome, Alexander's Disease, Lafora Disease, Charcot-Marie Tooth Disease, Prion Disease, a dementia, neurodegeneration, MeCP2 Duplication Syndrome, encephalopathy, neuroinflammation, or multiple sclerosis.
344. The oligomeric compound of embodiment 343, wherein the disorder or condition is Alzheimer's Disease.
345. The oligomeric compound of embodiment 343, wherein the disorder or condition is Amyotrophic Lateral Sclerosis.
346. The oligomeric compound of embodiment 343, wherein the disorder or condition is Parkinson's Disease.
347. The oligomeric compound of embodiment 343, wherein the disorder or condition is a Spinocerebellar ataxia.
348. The oligomeric compound of embodiment 343, wherein the disorder or condition is Angelman Syndrome.
349. The oligomeric compound of embodiment 343, wherein the disorder or condition is Alexander's Disease.
350. The oligomeric compound of embodiment 343, wherein the disorder or condition is Lafora Disease.
351. The oligomeric compound of embodiment 343, wherein the disorder or condition is Charcot-Marie Tooth Disease.
352. The oligomeric compound of embodiment 343, wherein the disorder or condition is Prion Disease.
353. The oligomeric compound of embodiment 343, wherein the disorder or condition is a dementia.
354. The oligomeric compound of embodiment 343, wherein the disorder or condition is neurodegeneration.
355. The oligomeric compound of embodiment 343, wherein the disorder or condition is MeCP2 Duplication Syndrome.
356. The oligomeric compound of embodiment 343, wherein the disorder or condition is encephalopathy.
357. The oligomeric compound of embodiment 343, wherein the disorder or condition is neuroinflammation.
358. The oligomeric compound of embodiment 343, wherein the disorder or condition is multiple sclerosis.
359. The oligomeric compound of any of embodiments 1-358, wherein the parent oligomeric compound of the oligomeric compound of any of embodiments 1-358 is cytotoxic in vitro.
360. The oligomeric compound of embodiment 359, wherein the parent oligomeric compound is cytotoxic in a standard in vitro cytotoxicity assay.
361. The oligomeric compound of any of embodiments 1-360, wherein administration to a mouse of the parent oligomeric compound of the oligomeric compound of any of embodiments 1-360 is hepatotoxic to the mouse.
362. The oligomeric compound of embodiment 361, wherein the administration of the parent oligomeric compound to the mouse is systemic administration.
363. The oligomeric compound of embodiment 362, wherein the systemic administration is subcutaneous administration.
364. The oligomeric compound of any of embodiments 361-363, wherein the mouse is a CD-1 mouse.
365. The oligomeric compound of any of embodiments 361-363, wherein the mouse is a C57BL/6 mouse.
366. The oligomeric compound of any of embodiments 361-363, wherein the mouse is a BALB/c mouse.
367. The oligomeric compound of any of embodiments 361-366, wherein the plasma ALT level in the mouse is higher than 500 units per liter following the administration of the parent oligomeric compound.
368. The oligomeric compound of any of embodiments 361-366, wherein the plasma AST level in the mouse is higher than 500 units per liter following the administration of the parent oligomeric compound.
369. The oligomeric compound of any of embodiments 361-366, wherein the plasma ALT level in the mouse is higher than 1000 units per liter following the administration of the parent oligomeric compound.
370. The oligomeric compound of any of embodiments 361-366, wherein the plasma AST level in the mouse is higher than 1000 units per liter following the administration of the parent oligomeric compound.
371. The oligomeric compound of any of embodiments 361-370, wherein the hepatotoxicity is measured 24-48 hours following the administration of the parent oligomeric compound.
372. The oligomeric compound of any of embodiments 361-370, wherein the hepatotoxicity is measured 72 hours following the administration of the parent oligomeric compound.
373. The oligomeric compound of any of embodiments 361-370, wherein the hepatotoxicity is measured 3 days following the administration of the parent oligomeric compound.
374. The oligomeric compound of any of embodiments 361-370, wherein the hepatotoxicity is measured 5 days following the administration of the parent oligomeric compound.
375. The oligomeric compound of any of embodiments 361-370, wherein the hepatotoxicity is measured 7 days following the administration of the parent oligomeric compound.
376. The oligomeric compound of any of embodiments 361-370, wherein the hepatotoxicity is measured 10 days following the administration of the parent oligomeric compound.
377. The oligomeric compound of any of embodiments 361-370, wherein the hepatotoxicity is measured 14 days following the administration of the parent oligomeric compound.
378. The oligomeric compound of any of embodiments 361-377, wherein 1-10 mg/kg of the parent oligomeric compound is administered to the mouse.
379. The oligomeric compound of any of embodiments 361-377, wherein 10-25 mg/kg of the parent oligomeric compound is administered to the mouse.
380. The oligomeric compound of any of embodiments 361-377, wherein 25-50 mg/kg of the parent oligomeric compound is administered to the mouse.
381. The oligomeric compound of any of embodiments 361-377, wherein 50-100 mg/kg of the parent oligomeric compound is administered to the mouse.
382. The oligomeric compound of any of embodiments 361-377, wherein 100-150 mg/kg of the parent oligomeric compound is administered to the mouse.
383. The oligomeric compound of any of embodiments 361-363, 366-370, 372, or 382, wherein the mouse is a BALB/c mouse, wherein 150 mg/kg of the parent oligomeric compound is administered to the mouse, and wherein the plasma ALT level in the mouse is measured 72 hours following the administration of the parent oligomeric compound.
384. The oligomeric compound of any of embodiments 1-383, wherein administration of 50 mg/kg of the oligomeric compound of any of embodiments 1-383 to a mouse is not hepatotoxic to the mouse.
385. The oligomeric compound of embodiment 384, wherein plasma ALT level in the mouse is lower than 100 units per liter following the administration of the oligomeric compound of embodiment 384.
386. The oligomeric compound of embodiment 384 or 385, wherein plasma ALT level in the mouse is at least 10-fold lower than plasma ALT level in a mouse administered the parent oligomeric compound of the oligomeric compound of embodiment 384 or 385, wherein the administrations and plasma ALT measurements of both the oligomeric compound of embodiment 384 or 385 and the parent oligomeric compound are completed in the same way.
387. The oligomeric compound of embodiment 386, wherein the plasma ALT level in the mouse is at least 100-fold lower than the plasma ALT level in the mouse administered the parent oligomeric compound.
388. The oligomeric compound of embodiment 386, wherein the plasma ALT level in the mouse is at least 500-fold lower than the plasma ALT level in the mouse administered the parent oligomeric compound.
389. The oligomeric compound of any of embodiments 359-388, wherein the therapeutic index in a mouse of the oligomeric compound of any of embodiments 359-388 is increased relative to the therapeutic index of the parent oligomeric compound.
390. The oligomeric compound of embodiment 389, wherein the therapeutic index in a mouse of the oligomeric compound of embodiment 365 is at least two-fold greater than the therapeutic index of the parent oligomeric compound.
391. The oligomeric compound of any of embodiments 1-390, wherein an otherwise identical oligomeric compound lacking the at least one modified internucleoside linkage other than phosphorothioate and/or at least one modified nucleoside comprising a modified nucleobase other than 5-methylcytosine and/or a modified sugar moiety in the central region is hepatotoxic following administration to a mouse; and wherein the otherwise identical oligomeric compound has a central region consisting of phosphorothioate internucleoside linkages, unmodified 2′-β-D-deoxyribosyl sugar moieties, and nucleobases selected from thymine, uracil, adenine, guanine, cytosine, and 5-methylcytosine.
392. The oligomeric compound of embodiment 391, wherein the at least one modified internucleoside linkage other than phosphorothioate and/or at least one modified nucleoside comprising a modified nucleobase other than 5-methylcytosine and/or a modified sugar moiety in the central region is a 2′-O-methyl modified sugar moiety.
393. The oligomeric compound of embodiment 392, wherein the 2′-O-methyl modified sugar moiety is at position 2 of the central region.
394. The oligomeric compound of embodiment 392 or 393, wherein the otherwise identical oligomeric compound lacks a 2′-O-methyl modified sugar moiety, and wherein the otherwise identical oligomeric compound comprises an unmodified 2′-β-D-deoxyribosyl sugar moiety in place of the 2′-O-methyl modified sugar moiety.
395. The oligomeric compound of any of embodiments 391-394, wherein the nucleobase sequence of the otherwise identical oligomeric compound is 100% complementary to the target RNA.
396. The oligomeric compound of any of embodiments 391-395, wherein the otherwise identical oligomeric compound is a parent oligomeric compound of the oligomeric compound of any of embodiments 391-395.
397. The oligomeric compound of any of embodiments 285-396, wherein the oligomeric compound inhibits the target RNA with an IC₅₀ lower than 100 nM in a standard in vitro activity assay.
398. The oligomeric compound of any of embodiments 285-397, wherein the parent oligomeric compound of the oligomeric compound of any of embodiments 285-397 inhibits the target RNA with an IC₅₀ lower than 100 nM in a standard in vitro activity assay.
399. The oligomeric compound of any of embodiments 285-398, wherein the difference between the IC₅₀ of the parent oligomeric compound measured in a standard in vitro activity assay and the IC₅₀ of the oligomeric compound of any of embodiments 285-398 measured in a standard in vitro activity assay is less than 4-fold.
400. The oligomeric compound of any of embodiments 285-398, wherein the difference between the IC₅₀ of the parent oligomeric compound measured in a standard in vitro activity assay and the IC₅₀ of the oligomeric compound of any of embodiments 285-398 measured in a standard in vitro activity assay is less than 3-fold.
401. The oligomeric compound of any of embodiments 285-398, wherein the difference between the IC₅₀ of the parent oligomeric compound measured in a standard in vitro activity assay and the IC₅₀ of the oligomeric compound of any of embodiments 285-398 measured in a standard in vitro activity assay is less than 2-fold.
402. The oligomeric compound of any of embodiments 359-390 or 396-401, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
403. The oligomeric compound of any of embodiments 359-390 or 396-402, wherein the nucleobase sequence of the parent oligomeric compound is 100% complementary to the target RNA.
404. The oligomeric compound of any of embodiments 1-403, wherein administration of 10-50 mg of the oligomeric compound to a human is not hepatotoxic to the human.
405. The oligomeric compound of any of embodiments 1-403, wherein administration of 50-100 mg of the oligomeric compound to a human is not hepatotoxic to the human.
406. The oligomeric compound of any of embodiments 1-403, wherein administration of 100-300 mg of the oligomeric compound to a human is not hepatotoxic to the human.
407. The oligomeric compound of any of embodiments 404-406, wherein the administration is systemic administration.
408. A composition comprising the oligomeric compound of any of embodiments 1-407 and a second oligomeric compound, wherein the second oligomeric compound is complementary to the oligomeric compound of any of embodiments 1-407.
409. The composition of embodiment 408, wherein the second oligomeric compound comprises at least 14 contiguous linked nucleosides that are at least 90% complementary to the oligomeric compound of any of embodiments 1-407.
410. The composition of embodiment 408, wherein the second oligomeric compound comprises at least 14 contiguous linked nucleosides that are 100% complementary to the oligomeric compound of any of embodiments 1-407.
411. A pharmaceutical composition comprising the oligomeric compound of any of embodiments 1-407 or the composition of any of embodiments 408-410, comprising a pharmaceutically acceptable carrier or diluent.
412. A method comprising administering the oligomeric compound or composition of any of embodiments 1-411 to a human subject.
413. A method of treating a disease or disorder comprising administering the oligomeric compound or composition of any of embodiments 1-411 to a human subject.
414. A method of modulating the amount or activity of a target RNA in a human subject comprising administering the oligomeric compound or composition of any of embodiments 1-411 to the human subject; and thereby modulating the amount or activity of the target RNA in the human subject.
415. A method of treating a disease or disorder in a human subject comprising administering the oligomeric compound or composition of any of embodiments 1-411 to the human subject having the disease or disorder; and thereby treating the disease or disorder in the human subject.
416. The method of embodiment 413 or 415, wherein the disease or disorder is a liver disease, and wherein the oligomeric compound modulates the amount or activity of a liver target RNA.
417. The method of embodiment 413 or 415, wherein the disease or disorder is a central nervous system disease, and wherein the oligomeric compound modulates the amount or activity of a central nervous system target RNA.
418. The method of any of embodiments 413 or 415-417, wherein the disease or disorder is a cancer, a neurodegenerative disease, a cardiovascular disease, an immunological disease, a hereditary disease, a repeat expansion disease, a muscle-wasting disease, or a metabolic disease.
419. The method of any of embodiments 412-418, wherein the plasma ALT level of the human subject after the administration is not more than three times higher than it was prior to the administration.
420. The method of any of embodiments 412-418, wherein the plasma ALT level of the human subject after the administration is not more than 150 units per liter.
421. The method of any of embodiments 412-416 or 418-420, wherein the human subject is susceptible to liver damage.
422. The method of any of embodiments 412-416 or 418-420, wherein the human subject is susceptible to liver degeneration.
423. The method of any of embodiments 412-416 or 418-420, wherein the human subject is susceptible to elevated apoptosis in the liver.
424. The method of any of embodiments 412-416 or 418-423, wherein the human subject has a liver disease.
425. The method of any of embodiments 412-424, comprising administering the parent oligomeric compound of the oligomeric compound of any of embodiments 1-407 to a mouse.
426. The method of any of embodiments 412-424, comprising contacting a cell with the parent oligomeric compound of the oligomeric compound of any of embodiments 1-407.
427. The method of embodiment 425 or 426, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
428. A method of designing an oligomeric compound comprising starting with a parent oligomeric compound and changing the design of that compound in order to arrive at an oligomeric compound of any one of embodiments 1-407.
429. A method of designing an oligomeric compound comprising identifying a parent oligomeric compound and changing the design of that parent oligomeric compound to arrive at a second oligomeric compound, wherein the second oligomeric compound is an oligomeric compound of any one of embodiments 1-407.
430. A method of improving hepatotoxicity of an oligomeric compound comprising the steps of (i) identifying a parent oligomeric compound that has plasma ALT levels above 300 units per liter in a mouse, and (ii) providing an oligomeric compound according to any one of embodiments 1-407.
431. The method of embodiment 428, wherein the method designs an oligomeric compound with improved therapeutic index relative to the parent oligomeric compound.
432. The method of embodiment 428 or 431, wherein the method designs an oligomeric compound with lower hepatotoxicity relative to the parent oligomeric compound.
433. The method of embodiment 429, wherein the second oligomeric compound has an improved therapeutic index relative to the parent oligomeric compound.
434. The method of embodiment 429 or 433, wherein the second oligomeric compound has reduced hepatotoxicity in a mouse relative to the parent oligomeric compound.
435. The method of embodiment 430, wherein the oligomeric compound according to any one of embodiments 1-407 has reduced hepatotoxicity relative to the parent oligomeric compound.
436. A method comprising administering an oligomeric compound of any of embodiments 1-407 to a mouse and separately administering the parent oligomeric compound of the oligomeric compound of any of embodiments 1-407 to a second mouse, wherein the therapeutic index of the oligomeric compound of any of embodiments 1-407 is improved relative to the therapeutic index of the parent oligomeric compound.
437. The method of any of embodiments 412-436, wherein the level of p21 RNA is measured in the mouse, the cell, and/or the human subject.
438. A method comprising administering an oligomeric compound to a subject and measuring the level of p21 RNA in the subject.
439. A method comprising administering an oligomeric compound of any of embodiments 1-407 to a subject and measuring the level of p21 RNA in the subject.
440. The method of embodiment 438 or 439, wherein the subject is a mouse.
441. The method of embodiment 438 or 439, wherein the subject is a human.
442. The method of any of embodiments 437-441, wherein the p21 RNA level is measured within 24 hours of the administration.
443. The method of any of embodiments 437-441, wherein the p21 RNA level is measured 24-48 hours following the administration.
444. An oligomeric compound or composition of any one of embodiments 1-411, for use in medical therapy.
445. A method comprising contacting a cell with an oligomeric compound and detecting the cellular localization of p54nrb protein in the cell.
446. The method of embodiment 445, comprising determining the relative amount of p54nrb protein in the nucleolus relative to other cells contacted with different oligomeric compounds.
447. The method of embodiment 445 or 446, comprising determining the relative amount of p54nrb in the nucleolus relative to the amount of p54nrb in the rest of the cell.
448. The method of any of embodiments 445-447, wherein the cell is in a plate containing at least 96 wells.
449. The method of any of embodiments 445-448, wherein the detection of the cellular localization of p54nrb comprises contacting the cell with a p54nrb antibody.
450. A method of screening for a tolerable oligomeric compound comprising any of the methods of embodiments 445-449.
451. The method of any of embodiments 445-450, wherein the oligomeric compound is the oligomeric compound of any of embodiments 1-407.
452. An oligomeric compound comprising a modified oligonucleotide consisting of 12-23 linked nucleosides, wherein the modified oligonucleotide comprises a 5′-region, a central region, and a 3′-region wherein:
   the 5′-region consists of 1-5 linked modified nucleosides;
   the 3′-region consists of 1-5 linked modified nucleosides; and
   the central region consists of 7-11 linked nucleosides and has the formula:

(N_{d1})(Nₓ)(N_{y})(N_{z})(N_{d})_{q}
wherein one of Nₓ, N_{y}, and N_{z},is a safety enhancing nucleoside;
the other two of Nₓ, N_{y}, and N_{z} are independently selected from an unmodified 2′-β-D-deoxyribosyl, a DNA isomer, and a DNA mimic;
N_{d1} and each N_{d} is independently selected from an unmodified 2′-β-D-deoxyribosyl, a DNA isomer, and a DNA mimic; and wherein
q is 2-7.
453. The oligomeric compound of embodiment 1, wherein the safety enhancing nucleoside is Nₓ or N_{y}.
454. The oligomeric compound of embodiment 1, wherein the safety enhancing nucleoside is Nₓ.
455. The oligomeric compound of embodiment 1, wherein the safety enhancing nucleoside is N_{y}.
456. The oligomeric compound of embodiment 1, wherein the safety enhancing nucleoside is N_{z}.
457. The oligomeric compound of any of embodiments 452-456, wherein the safety enhancing nucleoside has a sugar moiety selected from among a sugar surrogate, a bicyclic furanosyl sugar moiety, a non-bicyclic 2′-modified furanosyl sugar moiety, a non-bicyclic 4′-modified furanosyl sugar moiety, or a non-bicyclic 5′-modified furanosyl sugar moiety.
458. The oligomeric compound of any of embodiments 452-457, wherein the safety-enhancing nucleoside has a sugar moiety selected from among a morpholino, a 2′-O-methyl-2′-β-D-deoxyribosy sugar moiety, a cEt bicyclic sugar moiety, a LNA sugar moiety, an ENA sugar moiety, a 5′-methyl substituted 2′-deoxyribosyl sugar moiety, 5′-ethyl substituted 2′-deoxyribosyl sugar moiety, a 5′-allyl substituted 2′-deoxyribosyl sugar moiety and a 2′-β-L-deoxyxylosyl sugar moiety.
459. The oligomeric compound of any of embodiments 452-458, wherein the safety-enhancing nucleoside has a sugar moiety selected from among a 2′-O-methyl-modified sugar moiety and a 5′-modified sugar moiety.
460. The oligomeric compound of embodiment 459, wherein the safety-enhancing nucleoside has a 2′-O-methyl substituted ribosyl sugar moiety.
461. The oligomeric compound of any of embodiments 452-457, wherein the safety-enhancing nucleoside is a sugar surrogate.
462. The oligomeric compound of embodiment 461, wherein the sugar surrogate is selected from among a morpholino, a modified morpholino, and F-HNA.
463. The oligomeric compound of any of embodiments 452-462, wherein N_{d1} is an unmodified 2′-β-D-deoxyribosyl sugar moiety.
464. The oligomeric compound of any of embodiments 452-462, wherein Na_{d1} is a DNA isomer.
465. The oligomeric compound of any of embodiments 452-462, wherein Na_{d1} is a DNA mimic.
466. The oligomeric compound of any of embodiments 452-465, wherein no more than 3 of the central region nucleosides comprise a sugar moiety other than 2′-β-D-deoxyribosyl.
467. The oligomeric compound of any of embodiments 452-466, wherein each DNA isomer has a sugar moiety independently selected from among 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl.
468. The oligomeric compound of any of embodiments 452-467, wherein each DNA mimic has a sugar moiety is independently selected from among 5′-methyl-2′-β-D-deoxyribosyl and 5′-ethyl-2′-β-D-deoxyribosyl.
469. The oligomeric compound of any of embodiments 452-463, wherein each nucleoside of the central region other than the safety-enhancing nucleoside has a 2′-β-D-deoxyribosyl sugar moiety.
470. The oligomeric compound of any of 452-469, wherein at least one internucleoside linkage is a phosphorothioate linkage.
471. The oligomeric compound of any of embodiments 452-470, wherein at least 4 internucleoside linkages are phosphorothioate linkages.
472. The oligomeric compound of any of embodiments 452-471, wherein at least one internucleoside linkage is a neutral internucleoside linkage.
473. The oligomeric compound of any of embodiments 452-472, wherein at least one neutral internucleoside linkage is a phosphonate internucleoside linkage.
474. The oligomeric compound of any of embodiments 452-474, wherein at least one neutral internucleoside linkage is a methoxypropyl internucleoside linkage.
475. The oligomeric compound of any of embodiments 452-475, wherein at least one internucleoside linkage of the central region selected from among: the internucleoside Nd1 to Nx, Nx to Ny, Ny to Nz, or Nz to the adjacent Nd is a neutral internucleoside linkage.
476. The oligomeric compound of embodiment 475, wherein the modified oligonucleotide comprises one neutral linkage and the other internucleoside linkages are each independently selected from phosphodiester and phosphorothioate.
477. The oligomeric compound of any of embodiments 542-454 or embodiments 457-476, wherein the safety enhancing nucleoside is Nₓ and is a 2′O-methyl-substituted nucleoside.
478. The oligomeric compound of any of embodiments 2-453, 455, or embodiments 457-476, wherein the safety enhancing nucleoside is N_{y} and is a 2′O-methyl-substituted nucleoside.
479. The oligomeric compound of any of embodiments 452-453, 455, or embodiments 457-476, wherein the safety enhancing nucleoside is N_{y} and has a 5′-substituted 2′-deoxyribosyl sugar moiety.
480. The oligomeric compound of any of embodiments 452 or embodiments 457-476, wherein the safety enhancing nucleoside is N_{z} and has a 5′-substituted 2′-deoxyribosyl sugar moiety.
481. The oligomeric compound of embodiments 479 or 480, wherein the 5′-substituted ribosyl sugar moiety is a 5′-methyl, 5′-ethyl, or 5′-allyl substituted 2′-deoxyribosyl sugar moiety.
482. The oligomeric compound of embodiments 477-481, wherein at least one nucleoside of the 5′-region comprises a bicyclic furanosyl sugar moiety.
483. The oligomeric compound of any of embodiments 477-482, wherein each nucleoside of the 5′-region comprises a bicyclic furanosyl sugar moiety.
484. The oligomeric compound of any of embodiments 482 or 483, wherein each bicyclic sugar moiety of the 5′-region is selected from among cEt, LNA, and ENA.
485. The oligomeric compound of any of embodiments 452-482 or 484, wherein at least one nucleoside of the 5′-region comprises a non-bicyclic sugar moiety.
486. The oligomeric compound of embodiment 485, wherein the non-bicyclic sugar moiety of the at least one nucleoside of the 5′-region is a 2′-substituted ribosyl sugar moiety.
487. The oligomeric compound of any of embodiments 485-487, wherein each non-bicyclic sugar moiety of the 5′-region has a 2′-substituent selected from among 2′-MOE, 2′-OMe, and 2′-NMA.
488. The oligomeric compound of any of embodiments 477-480, wherein the 5′-region comprises a 2′-MOE modified nucleoside and an LNA modified nucleoside.
489. The oligomeric compound of any of embodiments 477-480, wherein the 5′-region comprises a 2′-MOE modified nucleoside and a cEt modified nucleoside.
490. The oligomeric compound of embodiments 477-489, wherein at least one nucleoside of the 3′-region comprises a bicyclic furanosyl sugar moiety.
491. The oligomeric compound of any of embodiments 477-490, wherein each nucleoside of the 3′-region comprises a bicyclic furanosyl sugar moiety.
492. The oligomeric compound of any of embodiments 490 or 491, wherein each bicyclic sugar moiety of the 3′-region is selected from among cEt, LNA, and ENA.
493. The oligomeric compound of any of embodiments 490 or 492, wherein at least one nucleoside of the 3′-region comprises a non-bicyclic sugar moiety.
494. The oligomeric compound of embodiment 493, wherein the non-bicyclic sugar moiety of the at least one nucleoside of the 3′-region is a 2′-substituted ribosyl sugar moiety.
495. The oligomeric compound of any of embodiments 490 or 492-494, wherein each non-bicyclic sugar moiety of the 3′-region has a 2′-substituent selected from among 2′-MOE, 2′-OMe, and 2′-NMA.
496. The oligomeric compound of any of embodiments 477-490 or 492-495, wherein the 3′-region comprises a 2′-MOE modified nucleoside and an LNA modified nucleoside.
497. The oligomeric compound of any of embodiments 477-490 or 492-495, wherein the 3′-region comprises a 2′-MOE modified nucleoside and a cEt modified nucleoside.
498. The oligomeric compound of any of embodiments 477-480, wherein each nucleoside of the 5′-region comprises a bicyclic furanosyl sugar moiety and each nucleoside of the 3′-region comprises a non-bicyclic sugar moiety.
499. The oligomeric compound of any of embodiments 477-480, wherein each nucleoside of the 5′-region comprises an LNA sugar moiety and each nucleoside of the 3′-region comprises a 2′MOE sugar moiety.
500. The oligomeric compound of any of embodiments 477-480, wherein each nucleoside of the 5′-region comprises cEt sugar moiety and each nucleoside of the 3′-region comprises a 2′MOE sugar moiety.
501. The oligomeric compound of any of embodiments 452-500, wherein the modified oligonucleotide has a nucleobase sequence complementary to a target RNA; wherein the target RNA is a mRNA or pre-mRNA.
502. The oligomeric compound of embodiment 501, wherein the target RNA encodes a protein that is expressed in the liver.
503. The oligomeric compound of embodiment 502, wherein the target RNA encodes a protein that is expressed in the CNS.
504. The oligomeric compound of any of embodiments 452-503, wherein the oligomeric compound is not toxic.
505. The oligomeric compound of any of embodiment 452-504, wherein a comparator compound is toxic; wherein the comparator compound is identical to the oligomeric compound except that each nucleoside of the central region of the comparator compound is a nucleoside comprising 2′-β-D-deoxyribosyl sugar moiety.
506. The oligomeric compound of embodiment 505, wherein administering a dose of the oligomeric compound to an animal results in a circulating ALT level that is at least 3-fold lower than the ALT level that results from administering the same dose of the comparator compound to an animal for at least one matched dose.
507. The oligomeric compound of embodiment 505 or 506, wherein the lowest dose of the oligomeric compound that raises circulating ALT to over 300 units per liter is at least three-fold higher than the lowest dose of the comparator compound that raises circulating ALT to over 300 units per liter.
508. The oligomeric compound of any of embodiments 502-507, wherein the oligomeric compound is capable of reducing the target RNA in a cell.
509. The oligomeric compound of embodiment 508, wherein the oligomeric compound has RNA-reducing activity comparable to a comparator compound, wherein the comparator compound is identical to the oligomeric compound except that each nucleoside of the central region of the comparator compound is a nucleoside comprising 2′-β-D-deoxyribosyl sugar moiety.
510. The oligomeric compound of embodiment 509 having RNA-reducing activity that is no more than 2-fold worse than the RNA-reducing activity of the comparator compound.
511. An oligomeric compound comprising a modified oligonucleotide consisting of 12-23 linked nucleosides, wherein the modified oligonucleotide comprises a 5′-region, a central region, and a 3′-region wherein:
   the 5′-region consists of 1-5 linked nucleosides; wherein at least one 5′-region nucleoside is modified;
   the 3′-region consists of 1-5 linked nucleosides; wherein at least one 3′-region nucleoside is modified; and
   the central region consists of 7-11 linked nucleosides, and has the formula:

(N_{d1})_{L1}(N_{d2})_{L2}(N_{d3})_{L3}(N_{d4})_{L4}(N_{d})_{q};
wherein N_{d1}, N_{d2}, N_{d3}, N_{d4}, and each N_{d} are independently selected from among a nucleoside comprising an unmodified 2′-β-D-deoxyribosyl sugar moiety, a DNA isomer sugar moiety, or a DNA mimic sugar moiety;
wherein each L1, L2, L3, and L4 is an internucleoside linkage; and wherein
at least one of L1, L2, L3, and L4 is a neutral internucleoside linkage.
512. The oligomeric compound of embodiment 511, wherein L1 is a neutral internucleoside linkage.
513. The oligomeric compound of embodiment 511, wherein L2 is a neutral internucleoside linkage.
514. The oligomeric compound of embodiment 511, wherein L3 is a neutral internucleoside linkage.
515. The oligomeric compound of any of embodiments 511-514, wherein the neutral linkage is a phosphonate internucleoside linkage.
516. The oligomeric compound of any of embodiments 511-515, wherein the neutral linkage is a methoxypropyl internucleoside linkage.
517. The oligomeric compound of any of embodiments 511-516, wherein each remaining internucleoside linkage is a phosphodiester or phosphorothioate internucleoside linkage.
518. A method comprising administering the oligomeric compound or composition of any of embodiments 452-517 to a human subject.
519. A method of treating a disease or disorder comprising administering the oligomeric compound or composition of any of embodiments 452-517 to a human subject.
520. A method of modulating the amount or activity of a target RNA in a human subject comprising administering the oligomeric compound or composition of any of embodiments 452-517 to the human subject; and thereby modulating the amount or activity of the target RNA in the human subject.
521. A method of treating a disease or disorder in a human subject comprising administering the oligomeric compound or composition of any of embodiments 452-517 to the human subject having the disease or disorder; and thereby treating the disease or disorder in the human subject.
522. The method of embodiment 520 or 521, wherein the disease or disorder is a liver disease, and wherein the oligomeric compound modulates the amount or activity of a liver target RNA.
523. The method of embodiment 520 or521, wherein the disease or disorder is a central nervous system disease, and wherein the oligomeric compound modulates the amount or activity of a central nervous system target RNA.
524. The method of any of embodiments 519-523, wherein the disease or disorder is a cancer, a neurodegenerative disease, a cardiovascular disease, an immunological disease, a hereditary disease, a repeat expansion disease, a muscle-wasting disease, or a metabolic disease.
525. The method of any of embodiments 518-524, wherein the plasma ALT level of the human subject after the administration is not more than three times higher than it was prior to the administration.
526. The method of any of embodiments 518-525, wherein the plasma ALT level of the human subject after the administration is not more than 150 units per liter.
527. The method of any of embodiments 518-526, wherein the human subject is susceptible to liver damage.
528. The method of any of embodiments 518-527, wherein the human subject is susceptible to liver degeneration.
529. The method of any of embodiments 518-528, wherein the human subject is susceptible to elevated apoptosis in the liver.
530. The method of any of embodiments 518-529, wherein the human subject has a liver disease.
531. The method of any of embodiments 518-530, comprising administering the parent oligomeric compound of the oligomeric compound of any of embodiments 452-517 to a mouse.
532. The method of any of embodiments 518-531, comprising contacting a cell with the parent oligomeric compound of the oligomeric compound of any of embodiments 452-517.
533. The method of embodiment 518-532, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
534. A method of designing an oligomeric compound comprising starting with a parent oligomeric compound and changing the design of that compound in order to arrive at an oligomeric compound of any one of embodiments 452-517.
535. A method of designing an oligomeric compound comprising identifying a parent oligomeric compound and changing the design of that parent oligomeric compound to arrive at a second oligomeric compound, wherein the second oligomeric compound is an oligomeric compound of any one of embodiments 452-517.
536. A method of improving hepatotoxicity of an oligomeric compound comprising the steps of (i) identifying a parent oligomeric compound that has plasma ALT levels above 300 units per liter in a mouse, and (ii) providing an oligomeric compound according to any one of embodiments 452-517.
537. The method of embodiment 536, wherein the method designs an oligomeric compound with improved therapeutic index relative to the parent oligomeric compound.
538. The method of embodiment 534-537, wherein the method designs an oligomeric compound with lower hepatotoxicity relative to the parent oligomeric compound.
539. The method of embodiment 535-538, wherein the second oligomeric compound has an improved therapeutic index relative to the parent oligomeric compound.
540. The method of embodiment 535-539, wherein the second oligomeric compound has reduced hepatotoxicity in a mouse relative to the parent oligomeric compound.
541. The method of embodiment 540, wherein the oligomeric compound according to any one of embodiments 452-517 has reduced hepatotoxicity relative to the parent oligomeric compound.
542. A method comprising administering an oligomeric compound of any of embodiments 452-517 to a mouse and separately administering the parent oligomeric compound of the oligomeric compound of any of embodiments 452-517 to a second mouse, wherein the therapeutic index of the oligomeric compound of any of embodiments 452-517 is improved relative to the therapeutic index of the parent oligomeric compound.
543. The method of any of embodiments 518-542, wherein the level of p21 RNA is measured in the mouse, the cell, and/or the human subject.
544. A method comprising administering an oligomeric compound of any of embodiments 452-517 to a subject and measuring the level of p21 RNA in the subject.
545. The method of embodiment 543 or 544, wherein the subject is a mouse.
546. The method of embodiment 543 or 544, wherein the subject is a human.
547. The method of any of embodiments 543-546, wherein the p21 RNA level is measured within 24 hours of the administration.
548. The method of any of embodiments 437-441, wherein the p21 RNA level is measured 24-48 hours following the administration.
549. An oligomeric compound or composition of any one of embodiments 452-517, for use in medical therapy.
550. The method of any of embodiments 445-449, wherein the oligomeric compound is the oligomeric compound of any of embodiments 452-517.
551. An oligomeric compound comprising a modified oligonucleotide consisting of 12-21 linked nucleosides, wherein the modified oligonucleotide has the formula A-B-C, wherein A is a 5′-region, B is a central region, and C is a 3′-region; wherein:
   the 5′-region consists of 1-5 linked nucleosides, wherein at least one nucleoside of the 5′-region is a modified nucleoside comprising a modified sugar;
   the 3′-region consists of 1-5 linked nucleosides wherein at least one nucleoside of the 3′-region is a modified nucleoside comprising a modified sugar; and
   the central region consists of 7-11 linked nucleosides, wherein
   the 5′-most portion of the central region has the following formula:

(N_{da})(Nₓ)(N_{y})(N_{z})(N_{db})
wherein one of Nₓ, N_{y}, and N_{z},is a safety-enhancing nucleoside;
the other two of Nₓ, N_{y}, and N_{z} are independently selected from a DNA nucleoside, a DNA isomer, a modified DNA isomer, and a DNA mimic; and
   N_{da} and N_{db} are each independently selected from a DNA nucleoside, a DNA isomer, a modified DNA isomer, and a DNA mimic.
552. The oligomeric compound of embodiment 551, wherein the 5′-region consists of one nucleoside.
553. The oligomeric compound of embodiment 551, wherein the 5′-region consists of 2-5 linked nucleosides.
554. The oligomeric compound of embodiment 551, wherein the 5′-region consists of 2-4 linked nucleosides.
555. The oligomeric compound of embodiment 551, wherein the 5′-region consists of 2 linked nucleosides.
556. The oligomeric compound of embodiment 551, wherein the 5′-region consists of 3 linked nucleosides.
557. The oligomeric compound of embodiment 551, wherein the 5′-region consists of 4 linked nucleosides.
558. The oligomeric compound of embodiment 551, wherein the 5′-region consists of 5 linked nucleosides.
559. The oligomeric compound of any of embodiments 551-558, wherein each nucleoside of the 5′-region is a modified nucleoside.
560. The oligomeric compound of any of embodiments 551-559, wherein each nucleoside of the 5′-region is a modified nucleoside comprising a modified sugar.
561. The oligomeric compound of any of embodiments 551-560, wherein each modified nucleoside of the 5′-region has the same modification.
562. The oligomeric compound of and of embodiments 551-560, wherein at least two nucleosides of the 5′-region are modified nucleosides having different modifications.
563. The oligomeric compound of any of embodiments 551-562, wherein each nucleoside of the 5′-region comprises a 2′-modified ribosyl sugar moiety.
564. The oligomeric compound of any of embodiments 551-563, wherein at least one nucleoside of the 5′-region comprises a bicyclic furanosyl sugar moiety.
565. The oligomeric compound of any of embodiments 551-564, wherein each nucleoside of the 5′-region comprises a bicyclic furanosyl sugar moiety.
566. The oligomeric compound of any of embodiments 551-565, wherein at least one nucleoside of the 5′-region comprises a non-bicyclic sugar moiety.
567. The oligomeric compound of embodiment 566, wherein the non-bicyclic sugar moiety of the at least one nucleoside of the 5′-region is a 2′-substituted ribosyl sugar moiety.
568. The oligomeric compound of any of embodiments 551-567, wherein each nucleoside of the 5′-region comprises a non-bicyclic sugar moiety.
569. The oligomeric compound of embodiment 568, wherein each nucleoside of the 5′-region comprises a 2′-substituted furanosyl sugar moiety.
570. The oligomeric compound of any of embodiments 551-569, wherein each nucleoside of the 5′-region comprises a 2′-modified furanosyl sugar moiety independently selected from a bicyclic sugar moiety and a non-bicyclic 2′-substituted ribosyl sugar moiety.
571. The oligomeric compound of any of embodiments 551-570, wherein each nucleoside of the 5′-region comprises a bicyclic sugar moiety.
572. The oligomeric compound of any of embodiments 551-571, wherein each nucleoside of the 5′-region comprises a non-bicyclic 2′-substituted ribosyl sugar moiety.
573. The oligomeric compound of any of embodiments 551-572, wherein each bicyclic sugar moiety of the 5′-region is selected from among cEt, LNA, and ENA.
574. The oligomeric compound of any of embodiments 551-573, wherein each bicyclic sugar moiety of the 5′-region is a cEt sugar moiety.
575. The oligomeric compound of any of embodiments 551-574, wherein each bicyclic sugar moiety of the 5′-region is an LNA sugar moiety.
576. The oligomeric compound of any of embodiments 551-575, wherein each non-bicyclic sugar moiety of the 5′-region has a 2′-substituent selected from among 2′-MOE, 2′-OMe, and 2′-NMA.
577. The oligomeric compound of any of embodiments 551-576, wherein none of the nucleosides of the 5′-region comprise a sugar moiety having a 2′-F substituent.
578. The oligomeric compound of any of embodiments 551-577, wherein none of the nucleosides of the 5′-region comprise a sugar moiety having a 2′-MOE substituent.
579. The oligomeric compound of any of embodiments 551-578, wherein none of the nucleosides of the 5′-region comprise a sugar moiety having a 2′-OMe substituent.
580. The oligomeric compound of any of embodiments 551-579, wherein none of the nucleosides of the 5′-region comprise a cEt sugar moiety.
581. The oligomeric compound of any of embodiments 551-580, wherein none of the nucleosides of the 5′-region comprise a LNA sugar moiety.
582. The oligomeric compound of any of embodiments 551-581, wherein each nucleobase of the 5′-region is independently selected from among thymine, uracil, guanine, cytosine, 5-methylcytosine, and adenine.
583. The oligomeric compound of any of embodiments 551-582, wherein each internucleoside linkage of the 5′-region is selected from among phosphodiester and phosphorothioate internucleoside linkages.
584. The oligomeric compound of any of embodiments 551-583, wherein the 3′-region consists of one nucleoside.
585. The oligomeric compound of any of embodiments 551-583, wherein the 3′-region consists of 2-5 linked nucleosides.
586. The oligomeric compound of any of embodiments 551-583, wherein the 3′-region consists of 2-4 linked nucleosides.
587. The oligomeric compound of any of embodiments 551-583, wherein the 3′-region consists of 2 linked nucleosides.
588. The oligomeric compound of any of embodiments 551-583, wherein the 3′-region consists of 3 linked nucleosides.
589. The oligomeric compound of any of embodiments 551-583, wherein the 3′-region consists of 4 linked nucleosides.
590. The oligomeric compound of any of embodiments 551-583, wherein the 3′-region consists of 5 linked nucleosides.
591. The oligomeric compound of any of embodiments 551-590, wherein each nucleoside of the 3′-region is a modified nucleoside.
592. The oligomeric compound of any of embodiments 551-591, wherein each nucleoside of the 3′-region is a modified nucleoside comprising a modified sugar moiety.
593. The oligomeric compound of any of embodiments 551-592, wherein each modified nucleoside of the 3′-region has the same modification.
594. The oligomeric compound of and of embodiments 551-592, wherein at least two nucleosides of the 3′-region are modified nucleosides having different modifications.
595. The oligomeric compound of any of embodiments 551-594, wherein each nucleoside of the 3′-region comprises a 2′-modified ribosyl sugar moiety.
596. The oligomeric compound of any of embodiments 551-595, wherein at least one nucleoside of the 3′-region comprises a bicyclic furanosyl sugar moiety.
597. The oligomeric compound of any of embodiments 551-596, wherein each nucleoside of the 3′-region comprises a bicyclic furanosyl sugar moiety.
598. The oligomeric compound of any of embodiments 551-597, wherein at least one nucleoside of the 3′-region comprises a non-bicyclic sugar moiety.
599. The oligomeric compound of embodiment 598, wherein the non-bicyclic sugar moiety of the at least one nucleoside of the 3′-region is a 2′-substituted ribosyl sugar moiety.
600. The oligomeric compound of any of embodiments 551-599, wherein each nucleoside of the 3′-region comprises a non-bicyclic sugar moiety.
601. The oligomeric compound of embodiment 600, wherein each nucleoside of the 3′-region comprises a 2′-substituted furanosyl sugar moiety.
602. The oligomeric compound of any of embodiments 551-601, wherein each nucleoside of the 3′-region comprises a 2′-modified furanosyl sugar moiety independently selected from a bicyclic sugar moiety and a non-bicyclic 2′-substituted ribosyl sugar moiety.
603. The oligomeric compound of any of embodiments 551-602, wherein each nucleoside of the 3′-region comprises a bicyclic sugar moiety.
604. The oligomeric compound of any of embodiments 551-602, wherein each nucleoside of the 3′-region comprises a non-bicyclic 2′-substituted ribosyl sugar moiety.
605. The oligomeric compound of any of embodiments 551-603, wherein each bicyclic sugar moiety of the 3′-region is selected from among cEt, LNA, and ENA.
606. The oligomeric compound of any of embodiments 551-605, wherein each bicyclic sugar moiety of the 3′-region is a cEt sugar moiety.
607. The oligomeric compound of any of embodiments 551-606, wherein each bicyclic sugar moiety of the 3′-region is an LNA sugar moiety.
608. The oligomeric compound of any of embodiments 551-607, wherein each non-bicyclic sugar moiety of the 3′-region has a 2′-substituent selected from among 2′-MOE, 2′-OMe, and 2′-NMA.
609. The oligomeric compound of any of embodiments 551-608, wherein none of the nucleosides of the 3′-region comprise a sugar moiety having a 2′-F substituent.
610. The oligomeric compound of any of embodiments 551-609, wherein none of the nucleosides of the 3′-region comprise a sugar moiety having a 2′-MOE substituent.
611. The oligomeric compound of any of embodiments 551-610, wherein none of the nucleosides of the 3′-region comprise a sugar moiety having a 2′-OMe substituent.
612. The oligomeric compound of any of embodiments 551-611, wherein none of the nucleosides of the 3′-region comprise a cEt sugar moiety.
613. The oligomeric compound of any of embodiments 551-612, wherein none of the nucleosides of the 3′-region comprise a LNA sugar moiety.
614. The oligomeric compound of any of embodiments 551-613, wherein each nucleobase of the 3′-region is independently selected from among thymine, uracil, guanine, cytosine, 5-methylcytosine, and adenine.
615. The oligomeric compound of any of embodiments 551-614, wherein each internucleoside linkage of the 3′-region is selected from among phosphodiester and phosphorothioate internucleoside linkages.
616. The oligomeric compound of any of embodiments 551-615, wherein the modified nucleosides of the 5′-region have the same modifications as the modifications of the modified nucleosides of the 3′-region.
617. The oligomeric compound of any of embodiments 551-615, wherein at least one modified nucleoside of the 5′-region and one modified nucleoside of the 3′-region comprise modifications that differ from one another.
618. The oligomeric compound of any of embodiments 551-617, wherein the 5′-region and the 3′-region together include at least one non-bicyclic 2′-substituted modified nucleoside and at least one bicyclic nucleoside.
619. The oligomeric compound of any of embodiment 618, where the bicyclic nucleoside is a cEt nucleoside.
620. The oligomeric compound of embodiment 618, where the bicyclic nucleoside is an LNA nucleoside.
621. The oligomeric compound of any of embodiments 618-620, wherein the non-bicyclic 2′-modified nucleoside is a 2′-MOE nucleoside.
622. The oligomeric compound of any of embodiments 618-620, wherein the non-bicyclic 2′-modified nucleoside is a 2′-OMe nucleoside.
623. The oligomeric compound of any of embodiments 618-622, wherein at least one nucleoside of the 5′-region or the 3′-region is an unmodified DNA nucleoside.
624. The oligomeric compound of any of embodiments 551-623, wherein the central region has the formula:

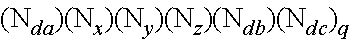

wherein each N_{dc} is independently selected from an unmodified DNA nucleoside, a DNA isomer, a 2′-modified DNA isomer, and a DNA mimic; and q is 2-6.
625. The oligomeric compound of any of embodiments 551-624, wherein the central region consists of 7 linked nucleosides.
626. The oligomeric compound of any of embodiments 551-624, wherein the central region consists of 8 linked nucleosides.
627. The oligomeric compound of any of embodiments 551-624, wherein the central region consists of 9 linked nucleosides.
628. The oligomeric compound of any of embodiments 551-624, wherein the central region consists of 10 linked nucleosides.
629. The oligomeric compound of any of embodiments 551-624, wherein the central region consists of 11 linked nucleosides.
630. The oligomeric compound of any of embodiments 551-629, wherein Nx is the safety-enhancing nucleoside.
631. The oligomeric compound of any of embodiments 551-629, wherein Ny is the safety-enhancing nucleoside.
632. The oligomeric compound of any of embodiments 551-629, wherein Nz is the safety-enhancing nucleoside.
633. The oligomeric compound of any of embodiments 551-632, wherein the safety enhancing nucleoside is a DNA isomer or a modified DNA isomer, or a modified nucleoside comprising either a sugar surrogate, a bicyclic furanosyl sugar moiety, or a non-bicyclic modified furanosyl sugar moiety.
634. The oligomeric compound of any of embodiments 551-33, wherein the safety enhancing nucleoside is a DNA isomer or a modified DNA isomer, or comprises either a sugar surrogate, a bicyclic furanosyl sugar moiety, a non-bicyclic, 2′-modified furanosyl sugar moiety, a non-bicyclic 3′-modified furanosyl sugar moiety, a non-bicyclic, 4′-modified furanosyl sugar moiety, or a non-bicyclic 5′-modified furanosyl sugar moiety.
635. The oligomeric compound of any of embodiments 551-634, wherein the safety enhancing nucleoside comprises a sugar surrogate selected from a morpholino, a modified morpholino, a modified cyclohexenyl, or a modified tetrahydropyran.
636. The oligomeric compound of any of embodiments 551-634, wherein the safety enhancing nucleoside comprises a sugar surrogate selected from a morpholino, a modified morpholino, CeNA, F-CeNA, HNA, OMe-HNA or F-HNA.
637. The oligomeric compound of any of embodiments 551-634, wherein the safety enhancing nucleoside is a DNA isomer comprising a sugar moiety selected from among 2′-β-D-deoxyxylosyl, 2′-α-L-deoxyribosyl, 2′-β-L-deoxyribosyl, 2′-α-D-deoxyxylosyl, 2′-α-D-deoxyribosyl, 2′-α-L-deoxyxylosyl, and 2′-β-L-deoxyxylosyl.
638. The oligomeric compound of any of embodiments 551-634, wherein the safety enhancing nucleoside is a DNA isomer comprising a sugar moiety selected from among 2′-β-D-deoxyxylosyl, 2′-α-L-deoxyribosyl, and 2′-β-L-deoxyribosyl.
639. The oligomeric compound of any of embodiments 551-634, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or O—C₁-C₁₀ alkoxy substituent.
640. The oligomeric compound of any of embodiments 551-634, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or OMe substituent.
641. The oligomeric compound of any of embodiments 551-634, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a fluoro or OMe substituent.
642. The oligomeric compound of any of embodiments 551-634, wherein the safety enhancing nucleoside is a modified DNA isomer comprising a sugar moiety selected from among 2′-β-D-deoxyxylosyl, 2′-α-L-deoxyribosyl, and 2′-β-L-deoxyribosyl and the 2′ position is further substituted with a hydroxyl, fluoro, or OMe substituent.
643. The oligomeric compound of any of embodiments 551-634, wherein the safety enhancing nucleoside is a modified nucleoside comprising a bicyclic furanosyl sugar moiety
644. The oligomeric compound of any of embodiments 551-634, wherein the safety enhancing nucleoside is selected from among cEt, LNA, α-L-LNA, and ENA.
645. The oligomeric compound of any of embodiments 551-634, wherein the safety enhancing nucleoside comprises a 2′-substitued furanosyl sugar moiety.
646. The oligomeric compound of embodiment 645, wherein the 2′-substitued furanosyl sugar moiety of the safety enhancing nucleoside has a 2′ substituent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
647. The oligomeric compound of embodiment 645, wherein the 2′-substitued furanosyl sugar moiety of the safety enhancing nucleoside has a 2′ substituent selected from: halo, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
648. The oligomeric compound of embodiment 645, wherein the 2′-substitued furanosyl sugar moiety of the safety enhancing nucleoside has a 2′ substituent selected from: fluoro, OMe, MOE, NMA.
649. The oligomeric compound of any of embodiments 551-648, wherein the safety enhancing nucleoside comprises a 2′-substitued furanosyl sugar moiety comprising a 2′-OMe or 2′-MOE.
650. The oligomeric compound of any of embodiments 551-649, wherein the safety enhancing nucleoside comprises a 2′-substitued furanosyl sugar moiety comprising a 2′-OMe.
651. The oligomeric compound of any of embodiments 551-650, wherein the safety enhancing nucleoside comprises a 2′-OMe modified 2′-β-D-deoxyribosyl sugar moiety.
652. The oligomeric compound of any of embodiments 551-634, wherein the safety enhancing nucleoside comprises a 3′-modified furanosyl sugar moiety having a 3′ substituent selected from: halo, allyl, amino, azido, SH, CN, CF₃, OCF₃, C₁-C₁₀ alkyl, C₁-C₁₀ substituted alkyl, S-alkyl, N(Rₘ)-alkyl, N(Rₘ)-alkenyl, N(Rₘ)-alkynyl, O-alkylenyl-O-alkyl, alkynyl, alkaryl, or aralkyl.
653. The oligomeric compound of any of embodiments 551-634 or embodiment 652, wherein the safety enhancing nucleoside comprises a 3′-modified furanosyl sugar moiety having a 3′ substituent selected from: C₁-C₁₀ alkyl, or C₁-C₁₀ substituted alkyl.
654. The oligomeric compound of any of embodiments 551-634 or 652-653, wherein the safety enhancing nucleoside comprises a 3′-methyl furanosyl sugar moiety.
655. The oligomeric compound of any of embodiments 551-634, wherein the safety enhancing nucleoside comprises a 4′-modified furanosyl sugar moiety having a 4′ substituent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
656. The oligomeric compound of any of embodiments 551-634 or 655, wherein the safety enhancing nucleoside comprises a 4′-modified furanosyl sugar moiety having a 4′-methyl.
657. The oligomeric compound of any of embodiments 551-634, wherein the safety enhancing nucleoside has the structure shown below, wherein R represents an optional 2′ substituent group and Bx is a heterocyclic base moiety:

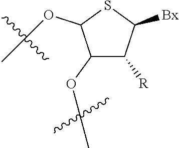

658. The oligomeric compound of embodiment 657, wherein in R is selected from among H, OH, OMe, F, or MOE.
659. The oligomeric compound of any of embodiments 551-634, wherein the safety enhancing nucleoside comprises a 5′-modified furanosyl sugar moiety having a 5′ substiuent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
660. The oligomeric compound of any of embodiments 551-634 or 659, wherein the safety enhancing nucleoside comprises a 5′-modified furanosyl sugar moiety with a 5′ substituent selected from: 5′-methyl, 5′-ethyl or a 5′-allyl.
661. The oligomeric compound of any of embodiments 551-634 or 659-660, wherein the safety enhancing nucleoside comprises a 5′-modified furanosyl sugar moiety with a 5′ substituent selected from: 5′-(R)-methyl- or 5′-(R)-ethyl.
662. The oligomeric compound of any of embodiments 551-634 or 659-661, wherein the safety enhancing nucleoside comprises a 5′-(R)-methyl-2′-β-D-deoxyribosyl sugar moiety.
663. The oligomeric compound of any of embodiments 551-634 or 659-662, wherein the safety enhancing nucleoside comprises a 5′-(R)-ethyl-2′-β-D-deoxyribosyl sugar moiety.
664. The oligomeric compound of any of embodiments 551-663, wherein the safety enhancing nucleoside does not comprise a 2′-F modified sugar moiety.
665. The oligomeric compound of any of embodiments 551-664, wherein the safety enhancing nucleoside does not comprise a cEt modified sugar moiety.
666. The oligomeric compound of any of embodiments 551-665, wherein the safety enhancing nucleoside does not comprise a 2′-MOE modified sugar moiety.
667. The oligomeric compound of any of embodiments 551-666, wherein the safety enhancing nucleoside comprises a hypoxanthine nucleobase.
668. The oligomeric compound of any of embodiments 551-667, wherein the safety enhancing nucleoside comprises a nucleobase selected from among A, T, G, C, ^{m}C, and U.
669. The oligomeric compound of any of embodiments 551-668, wherein the safety enhancing nucleoside is a modified nucleoside other than cEt, MOE, LNA, or FANA.
670. The oligomeric compound of any of embodiments 551-669, wherein each Nd is independently selected from among a DNA nucleoside, a DNA isomer, a modified DNA isomer, and a DNA mimic.
671. The oligomeric compound of embodiment 670, wherein each DNA isomer comprises a sugar moiety selected from among 2′-β-D-deoxyxylosyl, 2′-α-L-deoxyribosyl, 2′-β-L-deoxyribosyl, 2′-α-D-deoxyxylosyl, 2′-α-D-deoxyribosyl, 2′-α-L-deoxyxylosyl, and 2′-β-L-deoxyxylosyl.
672. The oligomeric compound of embodiment 671, wherein each DNA isomer comprises a sugar moiety selected from among 2′-β-D-deoxyxylosyl, 2′-α-L-deoxyribosyl, and 2′-β-L-deoxyribosyl.
673. The oligomeric compound of embodiment 670, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or O—C1-C10 alkoxy substituent.
674. The oligomeric compound of embodiment 673, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or OMe substituent.
675. The oligomeric compound of embodiment 674, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a fluoro or OMe substituent.
676. The oligomeric compound of embodiment 675, wherein each modified DNA isomer comprises a 2′-modified sugar moiety, wherein the sugar moiety is selected from among 2′-β-D-deoxyxylosyl, 2′-α-L-deoxyribosyl, and 2′-β-L-deoxyribosyl and the 2′ position is further substituted with a hydroxyl, fluoro, or OMe substituent.
677. The oligomeric compound of embodiment 670, wherein each DNA mimic comprises a structure represented by one of the formulas below:

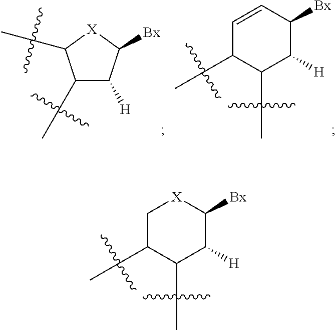

wherein X is O or S and Bx represents a heterocyclic base moiety.
678. The oligomeric compound of embodiment 670, wherein each DNA mimic comprises a structure represented by one of the formulas below:

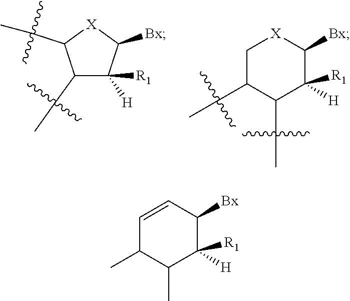

wherein X is O or S, Bx represents a heterocyclic base moiety, and R1 is selected from among H, OH, halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
679. The oligomeric compound of embodiment 678, wherein R1 is H, OH, OMe, or F.
680. The oligomeric compound of embodiment 678, wherein R1 is not F.
681. The oligomeric compound of embodiment 670, wherein each DNA mimic comprises a structure represented by the formula below:

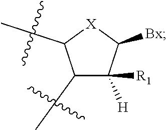

wherein X is O, Bx represents a heterocyclic base moiety, and R1 is H.
682. The oligomeric compound of embodiment 670, wherein DNA mimic has a sugar moiety selected from among: 5′-methyl-2′-β-D-deoxyribosyl, 5′-ethyl-2′-β-D-deoxyribosyl, and 5′-allyl-2′-β-D-deoxyribosyl.
683. The oligomeric compound of embodiment 670, wherein DNA mimic comprises a 2′-fluoro-β-D-arabinofuranosyl sugar moiety 684. The oligomeric compound of embodiment 670, wherein the DNA mimic does not comprise a 2′-fluoro-β-D-arabinofuranosyl sugar moiety.
685. The oligomeric compound of any of embodiments 551-684, wherein each Nd is a DNA nucleoside.
686. The oligomeric compound of any of embodiments 551-685, wherein the central region comprises no more than four nucleosides selected from among DNA isomers, modified DNA isomers, and DNA mimics.
687. The oligomeric compound of any of embodiments 551-685, wherein the central region comprises no more than three nucleosides selected from among DNA isomers, modified DNA isomers, and DNA mimics.
688. The oligomeric compound of any of embodiments 551-685, wherein the central region comprises no more than two nucleosides selected from among DNA isomers, modified DNA isomers, and DNA mimics.
689. The oligomeric compound of any of embodiments 551-685, wherein the central region comprises no more than one nucleoside selected from among DNA isomers, modified DNA isomers, and DNA mimics.
690. The oligomeric compound of any of embodiments 551-689, wherein the central region contains exactly one safety enhancing nucleoside and the remainder of nucleosides in the central region are DNA nucleosides.
691. The oligomeric compound of any of embodiments 551-690, wherein at least one internucleoside linkage of the central region selected from among: the internucleoside Nda to Nx, Nx to Ny, Ny to Nz, or Nz to the adjacent Ndb is a neutral internucleoside linkage.
692. The oligomeric compound of embodiments 691, wherein the neutral linkage is a phosphonate internucleoside linkage.
693. The oligomeric compound of embodiments 691, wherein the neutral linkage is a methoxypropyl internucleoside linkage.
694. The oligomeric compound of embodiments 691, wherein each remaining internucleoside linkage is a phosphodiester or phosphorothioate internucleoside linkage.
695. A chirally enriched population of modified oligonucleotides of any of embodiments 551-690, wherein the central region has at least one particular phorphorothioate internucleoside linkage having a particular stereochemical configuration.
696. The chirally enriched population of embodiment 695, wherein the central region has at least one phorphorothioate internucleoside linkage having the (Sp) configuration.
697. The chirally enriched population of embodiment 695, wherein central region has at least one phorphorothioate internucleoside linkage having the (Rp) configuration.
698. The chirally enriched population of embodiment 695, wherein the central region has a particular, independently selected stereochemical configuration at each phosphorothioate internucleoside linkage.
699. The chirally enriched population of embodiment 698, wherein the each phosphorothioate internucleoside linkage of the central region has the (Sp) configuration.
700. The chirally enriched population of embodiment 698, wherein the central region has one phosphorothioate internucleoside linkage having the (Rp) configuration and wherein each remaining central region phosphorothioate internucleoside linkage has the (Sp) configuration.
701. The chirally enriched population of embodiment 695, wherein the central region has an (Rp) phosphorothioate internucleoside linkage adjacent to an (Sp) phosphorothioate internucleoside linkage.
702. The chirally enriched population of embodiment 695, wherein the central region has an (Rp) phosphorothioate internucleoside linkage adjacent to a second (Rp) phosphorothioate internucleoside linkage.
703. The chirally enriched population of embodiment 695, wherein the central region has an (Rp) phosphorothioate internucleoside linkage adjacent to a second (Rp) phosphorothioate internucleoside linkage, and wherein each remaining central region phosphorothioate internucleoside linkage has the (Sp) configuration.
704. The chirally enriched population of any of embodiments 696, 697, 701, or 702 wherein each phosphorothioate internucleoside linkage that does not have the (Rp) or (Sp) configuration is stereorandom.
705. The oligomeric compound of any of embodiments 551-704 comprising a conjugate group.
706. The oligomeric compound of embodiment 705, wherein the conjugate group comprises a linking group attaching the remainder of the conjugate group to the modified oligonucleotide, wherein the linking group comprises 1-5 nucleosides.
707. The oligomeric compound of any of embodiments 1-705, wherein the oligomeric compound does not comprise additional nucleosides beyond those of the modified oligonucleotide.
708. The oligomeric compound of any of embodiments 551-707, wherein the nucleobase sequence of the modified oligonucleotide is complementary to a target RNA.
709. The oligomeric compound of embodiment 708, wherein the nucleobase sequence of the modified oligonucleotide is at least 85% complementary to the target RNA.
710. The oligomeric compound of embodiment 708, wherein the nucleobase sequence of the modified oligonucleotide is at least 90% complementary to the target RNA.
711. The oligomeric compound of embodiment 708, wherein the nucleobase sequence of the modified oligonucleotide is at least 95% complementary to the target RNA.
712. The oligomeric compound of embodiment 711, wherein the nucleobase sequence of the modified oligonucleotide is 100% complementary to the target RNA.
713. The oligomeric compound of any of embodiments 708-712, wherein the target RNA is a target mRNA or a target pre-mRNA.
714. The oligomeric compound of embodiment 713, wherein the target mRNA or target pre-mRNA is not a mouse, rat, monkey, or human PTEN RNA.
715. The oligomeric compound of embodiment 713 or 714, wherein the target mRNA or target pre-mRNA is not a mouse, rat, monkey, or human SRB-1 RNA.
716. The oligomeric compound of any of embodiments 713-715, wherein the target mRNA or target pre-mRNA is not a mouse, rat, monkey, or human MYD88 RNA.
717. The oligomeric compound of any of embodiments 713-716, wherein the target mRNA or target pre-mRNA is not a mouse, rat, monkey, or human HTT RNA, is not a mouse, rat, monkey, or human SOD1 RNA, and is not a mouse, rat, monkey, or human alpha-synuclein RNA.
718. The oligomeric compound of any of embodiments 708-717, wherein the target RNA is a human RNA.
719. The oligomeric compound of any of embodiments 708-718, wherein the target RNA is expressed in the liver.
720. The oligomeric compound of any of embodiments 708-719, wherein the target RNA is a liver target.
721. The oligomeric compound of any of embodiments 708-718, wherein the target RNA is not expressed in the liver.
722. The oligomeric compound of any of embodiments 708-718 or 721, wherein the target RNA is not a liver target.
723. The oligomeric compound of any of embodiments 708-722, wherein modulation of the expression of the target RNA in the liver is associated with treating a disorder or condition.
724. The oligomeric compound of embodiment 723, wherein the disorder or condition is a liver disorder or condition.
725. The oligomeric compound of any of embodiments 708-724, wherein the target RNA is expressed in the central nervous system.
726. The oligomeric compound of any of embodiments 708-724, wherein the target RNA is not expressed in the central nervous system.
727. The oligomeric compound of any of embodiments 708-725, wherein the target RNA is a central nervous system target.
728. The oligomeric compound of any of embodiments 708-726, wherein the target RNA is not a central nervous system target.
729. The oligomeric compound of any of embodiments 708-727, wherein the target RNA is a MeCP2, DUX4, HDAC2, APP, Ataxin 1, Ataxin 2, Ataxin 3, Ataxin 6, Ataxin 7, C9ORF72, UBE3A, Prion, PMP22, Tau, LRRK2, LINGO2, GYS1, KCNT1, IRF8, Progranulin, or GFAP RNA.
730. The oligomeric compound of any of embodiments 708-729, wherein the target RNA is a HTT RNA.
731. The oligomeric compound of embodiment 729, wherein the target RNA is a MeCP2 RNA.
732. The oligomeric compound of embodiment 729, wherein the target RNA is a DUX4 RNA.
733. The oligomeric compound of embodiment 729, wherein the target RNA is a HDAC2 RNA.
734. The oligomeric compound of embodiment 729, wherein the target RNA is a Ataxin 1 RNA.
735. The oligomeric compound of embodiment 729, wherein the target RNA is a Ataxin 2 RNA.
736. The oligomeric compound of embodiment 729, wherein the target RNA is a Ataxin 3 RNA.
737. The oligomeric compound of embodiment 729, wherein the target RNA is a Ataxin 6 RNA.
738. The oligomeric compound of embodiment 729, wherein the target RNA is a Ataxin 7 RNA.
739. The oligomeric compound of embodiment 729, wherein the target RNA is a C9ORF72 RNA.
740. The oligomeric compound of embodiment 708-727, wherein the target RNA is an alpha-synuclein RNA.
741. The oligomeric compound of embodiment 729, wherein the target RNA is an UBE3A RNA.
742. The oligomeric compound of any of embodiments 708-727, wherein the target RNA is a SOD1 RNA.
743. The oligomeric compound of any of embodiments 708-727, wherein the target RNA is a Prion RNA.
744. The oligomeric compound of any of embodiments 708-727, wherein the target RNA is a PMP22 RNA.
745. The oligomeric compound of any of embodiments 708-727, wherein the target RNA is a Tau RNA.
746. The oligomeric compound of any of embodiments 708-727, wherein the target RNA is a LRRK2 RNA.
747. The oligomeric compound of any of embodiments 708-727, wherein the target RNA is an APP RNA.
748. The oligomeric compound of any of embodiments 708-727, wherein the target RNA is a LINGO2 RNA.
749. The oligomeric compound of any of embodiments 708-727, wherein the target RNA is a GYS1 RNA.
750. The oligomeric compound of any of embodiments 708-727, wherein the target RNA is a KCNT1 RNA.
751. The oligomeric compound of any of embodiments 708-727, wherein the target RNA is a IRF8 RNA.
752. The oligomeric compound of any of embodiments 708-727, wherein the target RNA is a Progranulin RNA.
753. The oligomeric compound of any of embodiments 708-727, wherein the target RNA is a GFAP RNA.
754. The oligomeric compound of any of embodiments 725-753, wherein modulation of the expression of the target RNA in the central nervous system is associated with treating a disorder or condition.
755. The oligomeric compound of embodiment 754, wherein the disorder or condition is a neurological disorder or condition.
756. The oligomeric compound of embodiment 754-755, wherein the disorder or condition alters the function of sensory or motor neurons.
757. The oligomeric compound of any of embodiments 754-756, wherein the disorder or condition alters the function of sensory neurons.
758. The oligomeric compound of any of embodiments 754-757, wherein the disorder or condition alters the function of motor neurons.
759. The oligomeric compound of any of embodiments 754-758, wherein the disorder or condition alters the function of glial cells.
760. The oligomeric compound of any of embodiments 754-759, wherein the disorder or condition alters the function of astrocytes.
761. The oligomeric compound of any of embodiments 754-760, wherein the disorder or condition alters the function of oligodendrocytes.
762. The oligomeric compound of any of embodiments 754-761, wherein the disorder or condition alters the function of microglia.
763. The oligomeric compound of any of embodiments 754-762, wherein the disorder or condition alters the function of ependymal cells.
764. The oligomeric compound of any of embodiments 754-763, wherein the disorder or condition is Alzheimer's Disease, Amyotrophic Lateral Sclerosis, Parkinson's Disease, a Spinocerebellar ataxia, Angelman Syndrome, Alexander's Disease, Lafora Disease, Charcot-Marie Tooth Disease, Prion Disease, a dementia, neurodegeneration, MeCP2 Duplication Syndrome, encephalopathy, neuroinflammation, or multiple sclerosis.
765. The oligomeric compound of embodiment 764, wherein the disorder or condition is Alzheimer's Disease.
766. The oligomeric compound of embodiment 764, wherein the disorder or condition is Amyotrophic Lateral Sclerosis.
767. The oligomeric compound of embodiment 764, wherein the disorder or condition is Parkinson's Disease.
768. The oligomeric compound of embodiment 764, wherein the disorder or condition is a Spinocerebellar ataxia.
769. The oligomeric compound of embodiment 764, wherein the disorder or condition is Angelman Syndrome.
770. The oligomeric compound of embodiment 764, wherein the disorder or condition is Alexander's Disease.
771. The oligomeric compound of embodiment 764, wherein the disorder or condition is Lafora Disease.
772. The oligomeric compound of embodiment 764, wherein the disorder or condition is Charcot-Marie Tooth Disease.
773. The oligomeric compound of embodiment 764, wherein the disorder or condition is Prion Disease.
774. The oligomeric compound of embodiment 764, wherein the disorder or condition is a dementia.
775. The oligomeric compound of embodiment 764, wherein the disorder or condition is neurodegeneration.
776. The oligomeric compound of embodiment 764, wherein the disorder or condition is MeCP2 Duplication Syndrome.
777. The oligomeric compound of embodiment 764, wherein the disorder or condition is encephalopathy.
778. The oligomeric compound of embodiment 764, wherein the disorder or condition is neuroinflammation.
779. The oligomeric compound of embodiment 764, wherein the disorder or condition is multiple sclerosis.
780. The oligomeric compound of any of embodiments 551-779, wherein the parent oligomeric compound of the oligomeric compound of any of embodiments 551-779 is cytotoxic in vitro.
781. The oligomeric compound of embodiment 780, wherein the parent oligomeric compound is cytotoxic in a standard in vitro cytotoxicity assay.
782. The oligomeric compound of any of embodiments 551-781 wherein administration to a mouse of the parent oligomeric compound of the oligomeric compound of any of embodiments 551-781 is hepatotoxic to the mouse.
783. The oligomeric compound of embodiment 782, wherein the administration of the parent oligomeric compound to the mouse is systemic administration.
784. The oligomeric compound of embodiment 783, wherein the systemic administration is subcutaneous administration.
785. The oligomeric compound of any of embodiments 782-784, wherein the mouse is a CD-1 mouse.
786. The oligomeric compound of any of embodiments 782-784, wherein the mouse is a C57BL/6 mouse.
787. The oligomeric compound of any of embodiments 782-784, wherein the mouse is a BALB/c mouse.
788. The oligomeric compound of any of embodiments 782-784, wherein the plasma ALT level in the mouse is higher than 500 units per liter following the administration of the parent oligomeric compound.
789. The oligomeric compound of any of embodiments 782-788, wherein the plasma AST level in the mouse is higher than 500 units per liter following the administration of the parent oligomeric compound.
790. The oligomeric compound of any of embodiments 782-789, wherein the plasma ALT level in the mouse is higher than 1000 units per liter following the administration of the parent oligomeric compound.
791. The oligomeric compound of any of embodiments 782-790, wherein the plasma AST level in the mouse is higher than 1000 units per liter following the administration of the parent oligomeric compound.
792. The oligomeric compound of any of embodiments 782-791, wherein the hepatotoxicity is measured 24-48 hours following the administration of the parent oligomeric compound.
793. The oligomeric compound of any of embodiments 782-791, wherein the hepatotoxicity is measured 72 hours following the administration of the parent oligomeric compound.
794. The oligomeric compound of any of embodiments 782-791, wherein the hepatotoxicity is measured 3 days following the administration of the parent oligomeric compound.
795. The oligomeric compound of any of embodiments 782-791, wherein the hepatotoxicity is measured 5 days following the administration of the parent oligomeric compound.
796. The oligomeric compound of any of embodiments 782-791, wherein the hepatotoxicity is measured 7 days following the administration of the parent oligomeric compound.
797. The oligomeric compound of any of embodiments 782-791, wherein the hepatotoxicity is measured 10 days following the administration of the parent oligomeric compound.
798. The oligomeric compound of any of embodiments 782-791, wherein the hepatotoxicity is measured 14 days following the administration of the parent oligomeric compound.
799. The oligomeric compound of any of embodiments 782-791, wherein 1-10 mg/kg of the parent oligomeric compound is administered to the mouse.
800. The oligomeric compound of any of embodiments 782-791, wherein 10-25 mg/kg of the parent oligomeric compound is administered to the mouse.
801. The oligomeric compound of any of embodiments 782-791, wherein 25-50 mg/kg of the parent oligomeric compound is administered to the mouse.
802. The oligomeric compound of any of embodiments 782-791, wherein 50-100 mg/kg of the parent oligomeric compound is administered to the mouse.
803. The oligomeric compound of any of embodiments 782-791, wherein 100-150 mg/kg of the parent oligomeric compound is administered to the mouse.
804. The oligomeric compound of any of embodiments 782-791, wherein the mouse is a BALB/c mouse, wherein 150 mg/kg of the parent oligomeric compound is administered to the mouse, and wherein the plasma ALT level in the mouse is measured 72 hours following the administration of the parent oligomeric compound.
805. The oligomeric compound of any of embodiments 551-804, wherein administration of 50 mg/kg of the oligomeric compound of any of embodiments 551-804 to a mouse is not hepatotoxic to the mouse.
806. The oligomeric compound of embodiment 805, wherein plasma ALT level in the mouse is lower than 100 units per liter following the administration of the oligomeric compound of embodiment 805.
807. The oligomeric compound of embodiment 805 or 806, wherein plasma ALT level in the mouse is at least 10-fold lower than plasma ALT level in a mouse administered the parent oligomeric compound of the oligomeric compound of embodiment 384 or 385, wherein the administrations and plasma ALT measurements of both the oligomeric compound of embodiment 384 or 385 and the parent oligomeric compound are completed in the same way.
808. The oligomeric compound of embodiment 807, wherein the plasma ALT level in the mouse is at least 100-fold lower than the plasma ALT level in the mouse administered the parent oligomeric compound.
809. The oligomeric compound of embodiment 807, wherein the plasma ALT level in the mouse is at least 500-fold lower than the plasma ALT level in the mouse administered the parent oligomeric compound.
810. The oligomeric compound of any of embodiments 782-809, wherein the therapeutic index in a mouse of the oligomeric compound of any of embodiments 782-809 is increased relative to the therapeutic index of the parent oligomeric compound.
811. The oligomeric compound of embodiment 810, wherein the therapeutic index in a mouse of the oligomeric compound of embodiment 365 is at least two-fold greater than the therapeutic index of the parent oligomeric compound.
812. The oligomeric compound of any of embodiments 551-811, wherein an otherwise identical oligomeric compound lacking the at least one modified internucleoside linkage other than phosphorothioate and/or at least one modified nucleoside comprising a modified nucleobase other than 5-methylcytosine and/or a modified sugar moiety in the central region is hepatotoxic following administration to a mouse; and wherein the otherwise identical oligomeric compound has a central region consisting of phosphorothioate internucleoside linkages, unmodified 2′-β-D-deoxyribosyl sugar moieties, and nucleobases selected from thymine, uracil, adenine, guanine, cytosine, and 5-methylcytosine.
813. The oligomeric compound of embodiment 812, wherein the at least one modified internucleoside linkage other than phosphorothioate and/or at least one modified nucleoside comprising a modified nucleobase other than 5-methylcytosine and/or a modified sugar moiety in the central region is a 2′-O-methyl modified sugar moiety.
814. The oligomeric compound of embodiment 813, wherein the 2′-O-methyl modified sugar moiety is at position 2 of the central region.
815. The oligomeric compound of embodiment 812-814, wherein the otherwise identical oligomeric compound lacks a 2′-O-methyl modified sugar moiety, and wherein the otherwise identical oligomeric compound comprises an unmodified 2′-β-D-deoxyribosyl sugar moiety in place of the 2′-O-methyl modified sugar moiety.
816. The oligomeric compound of any of embodiments 812-815, wherein the nucleobase sequence of the otherwise identical oligomeric compound is 100% complementary to the target RNA.
817. The oligomeric compound of any of embodiments 812-816, wherein the otherwise identical oligomeric compound is a parent oligomeric compound of the oligomeric compound of any of embodiments 812-816.
818. The oligomeric compound of any of embodiments 708-817, wherein the oligomeric compound inhibits the target RNA with an IC₅₀ lower than 100 nM in a standard in vitro activity assay.
819. The oligomeric compound of any of embodiments 708-818, wherein the parent oligomeric compound of the oligomeric compound of any of embodiments 708-818 inhibits the target RNA with an IC₅₀ lower than 100 nM in a standard in vitro activity assay.
820. The oligomeric compound of any of embodiments 708-819, wherein the difference between the IC₅₀ of the parent oligomeric compound measured in a standard in vitro activity assay and the IC₅₀ of the oligomeric compound of any of embodiments 708-819 measured in a standard in vitro activity assay is less than 4-fold.
821. The oligomeric compound of any of embodiments 708-820, wherein the difference between the IC₅₀ of the parent oligomeric compound measured in a standard in vitro activity assay and the IC₅₀ of the oligomeric compound of any of embodiments 708-820 measured in a standard in vitro activity assay is less than 3-fold.
822. The oligomeric compound of any of embodiments 708-821, wherein the difference between the IC₅₀ of the parent oligomeric compound measured in a standard in vitro activity assay and the IC₅₀ of the oligomeric compound of any of embodiments 708-821 measured in a standard in vitro activity assay is less than 2-fold.
823. The oligomeric compound of any of embodiments 708-822, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
824. The oligomeric compound of any of embodiments 708-823, wherein the nucleobase sequence of the parent oligomeric compound is 100% complementary to the target RNA.
825. The oligomeric compound of any of embodiments 551-824, wherein administration of 10-50 mg of the oligomeric compound to a human is not hepatotoxic to the human.
826. The oligomeric compound of any of embodiments 551-824, wherein administration of 50-100 mg of the oligomeric compound to a human is not hepatotoxic to the human.
827. The oligomeric compound of any of embodiments 551-824, wherein administration of 100-300 mg of the oligomeric compound to a human is not hepatotoxic to the human.
828. The oligomeric compound of any of embodiments 825-827, wherein the administration is systemic administration.
829. A composition comprising the oligomeric compound of any of embodiments 551-828 and a second oligomeric compound, wherein the second oligomeric compound is complementary to the oligomeric compound of any of embodiments 551-828.
830. The composition of embodiment 829, wherein the second oligomeric compound comprises at least 14 contiguous linked nucleosides that are at least 90% complementary to the oligomeric compound of any of embodiments 551-828.
831. The composition of embodiment 830, wherein the second oligomeric compound comprises at least 14 contiguous linked nucleosides that are 100% complementary to the oligomeric compound of any of embodiments 551-828.
832. A pharmaceutical composition comprising the oligomeric compound of any of embodiments 551-828 or the composition of any of embodiments 829-831, comprising a pharmaceutically acceptable carrier or diluent.
833. A method comprising administering the oligomeric compound or composition of any of embodiments 551-832 to a human subject.
834. A method of treating a disease or disorder comprising administering the oligomeric compound or composition of any of embodiments 551-832 to a human subject.
835. A method of modulating the amount or activity of a target RNA in a human subject comprising administering the oligomeric compound or composition of any of embodiments 551-832 to the human subject; and thereby modulating the amount or activity of the target RNA in the human subject.
836. A method of treating a disease or disorder in a human subject comprising administering the oligomeric compound or composition of any of embodiments 551-832 to the human subject having the disease or disorder; and thereby treating the disease or disorder in the human subject.
837. The method of embodiment 835-836, wherein the disease or disorder is a liver disease, and wherein the oligomeric compound modulates the amount or activity of a liver target RNA.
838. The method of embodiment 835-837, wherein the disease or disorder is a central nervous system disease, and wherein the oligomeric compound modulates the amount or activity of a central nervous system target RNA.
839. The method of any of embodiments 834-838, wherein the disease or disorder is a cancer, a neurodegenerative disease, a cardiovascular disease, an immunological disease, a hereditary disease, a repeat expansion disease, a muscle-wasting disease, or a metabolic disease.
840. The method of any of embodiments 834-839, wherein the plasma ALT level of the human subject after the administration is not more than three times higher than it was prior to the administration.
841. The method of any of embodiments 834-840, wherein the plasma ALT level of the human subject after the administration is not more than 150 units per liter.
842. The method of any of embodiments 834-841, wherein the human subject is susceptible to liver damage.
843. The method of any of embodiments 834-842, wherein the human subject is susceptible to liver degeneration.
844. The method of any of embodiments 834-843, wherein the human subject is susceptible to elevated apoptosis in the liver.
845. The method of any of embodiments 834-844, wherein the human subject has a liver disease.
846. The method of any of embodiments 834-841, comprising administering the parent oligomeric compound of the oligomeric compound of any of embodiments 551-832 to a mouse.
847. The method of any of embodiments 833-846, comprising contacting a cell with the parent oligomeric compound of the oligomeric compound of any of embodiments 551-832.
848. The method of embodiment 846-847, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
849. A method of designing an oligomeric compound comprising starting with a parent oligomeric compound and changing the design of that compound in order to arrive at an oligomeric compound of any one of embodiments 551-833.
850. A method of designing an oligomeric compound comprising identifying a parent oligomeric compound and changing the design of that parent oligomeric compound to arrive at a second oligomeric compound, wherein the second oligomeric compound is an oligomeric compound of any one of embodiments 551-833.
851. A method of improving hepatotoxicity of an oligomeric compound comprising the steps of (i) identifying a parent oligomeric compound that has plasma ALT levels above 300 units per liter in a mouse, and (ii) providing an oligomeric compound according to any one of embodiments 551-833.
852. The method of embodiment 851, wherein the method designs an oligomeric compound with improved therapeutic index relative to the parent oligomeric compound.
853. The method of embodiment 851-852, wherein the method designs an oligomeric compound with lower hepatotoxicity relative to the parent oligomeric compound.
854. The method of embodiment 853, wherein the second oligomeric compound has an improved therapeutic index relative to the parent oligomeric compound.
855. The method of embodiment 851-852, wherein the second oligomeric compound has reduced hepatotoxicity in a mouse relative to the parent oligomeric compound.
856. The method of embodiment 855, wherein the oligomeric compound according to any one of embodiments 551-833 has reduced hepatotoxicity relative to the parent oligomeric compound.
857. A method comprising administering an oligomeric compound of any of embodiments 551-833 to a mouse and separately administering the parent oligomeric compound of the oligomeric compound of any of embodiments 551-833 to a second mouse, wherein the therapeutic index of the oligomeric compound of any of embodiments 551-833is improved relative to the therapeutic index of the parent oligomeric compound.
858. The method of any of embodiments 833-857, wherein the level of p21 RNA is measured in the mouse, the cell, and/or the human subject.
859. A method comprising administering an oligomeric compound of any of embodiments 551-833 to a subject and measuring the level of p21 RNA in the subject.
860. The method of embodiment 858 or 859, wherein the subject is a mouse.
861. The method of embodiment 858 or 859, wherein the subject is a human.
862. The method of any of embodiments 858-861, wherein the p21 RNA level is measured within 24 hours of the administration.
863. The method of any of embodiments 858-862, wherein the p21 RNA level is measured 24-48 hours following the administration.
864. An oligomeric compound or composition of any one of embodiments 551-832, for use in medical therapy.
865. The oligomeric compound of any of embodiments 551-832, wherein the oligomeric compound is not toxic.
866. The oligomeric compound of any of embodiment 551-832, wherein a comparator compound is toxic; wherein the comparator compound is identical to the oligomeric compound except that each nucleoside of the central region of the comparator compound is a nucleoside comprising 2′-β-D-deoxyribyl sugar moiety.
867. The oligomeric compound of embodiment 866, wherein administering a dose of the oligomeric compound to an animal results in a circulating ALT level that is at least 3-fold lower than the ALT level that results from administering the same dose of the comparator compound to an animal for at least one matched dose.
868. The oligomeric compound of embodiment 866 or 867, wherein the lowest dose of the oligomeric compound that raises circulating ALT to over 300 units per liter is at least three-fold higher than the lowest dose of the comparator compound that raises circulating ALT to over 300 units per liter.
869. The oligomeric compound of any of embodiments 865-868, wherein the oligomeric compound is capable of reducing the target RNA in a cell.
870. The oligomeric compound of embodiment 869, wherein the oligomeric compound has RNA-reducing activity comparable to a comparator compound, wherein the comparator compound is identical to the oligomeric compound except that each nucleoside of the central region of the comparator compound is a nucleoside comprising 2′-β-D-deoxyribosyl sugar moiety.
871. The oligomeric compound of embodiment 870 having RNA-reducing activity that is no more than 2-fold worse than the RNA-reducing activity of the comparator compound.
872. A method of modulating the amount or activity of a target RNA in a human subject comprising administering the oligomeric compound or composition of any of embodiments 551-832 to the human subject; and thereby modulating the amount or activity of the target RNA in the human subject.
873. A method of treating a disease or disorder in a human subject comprising administering the oligomeric compound or composition of any of embodiments 551-832 to the human subject having the disease or disorder; and thereby treating the disease or disorder in the human subject.
874. The method of embodiment 872-873, wherein the disease or disorder is a liver disease, and wherein the oligomeric compound modulates the amount or activity of a liver target RNA.
875. The method of embodiment 872-873, wherein the disease or disorder is a central nervous system disease, and wherein the oligomeric compound modulates the amount or activity of a central nervous system target RNA.
876. The method of any of embodiments 872-874, wherein the disease or disorder is a cancer, a neurodegenerative disease, a cardiovascular disease, an immunological disease, a hereditary disease, a repeat expansion disease, a muscle-wasting disease, or a metabolic disease.
877. The method of any of embodiments 872-876, wherein the plasma ALT level of the human subject after the administration is not more than three times higher than it was prior to the administration.
878. The method of any of embodiments 872-877, wherein the plasma ALT level of the human subject after the administration is not more than 150 units per liter.
879. A method of treating a disease or disorder in a human subject comprising administering the oligomeric compound or composition of any of embodiments 1-410, 452-518, 551-828, or 864-871 to the human subject having the disease or disorder; and thereby treating the disease or disorder in the human subject.
880. The method of embodiment 879, wherein the disease or disorder is not a CNS disease or disorder.
881. The method of embodiment 879, wherein the disease or disorder is a disease or disorder of the muscle.
882. The method of embodiment 879, wherein the disease or disorder is a disease or disorder of the lung.
883. The method of embodiment 879, wherein the disease or disorder is a disease or disorder of the kidney.
884. The method of embodiment 879, wherein the disease or disorder is a disease or disorder of the eye.
885. The method of embodiment 879, wherein the disease or disorder is a disease or disorder of the pancreas.
886. A method of screening a library of oligomeric compounds for activity against a target RNA, wherein the library of oligomeric compounds comprises a plurality of oligomeric compounds of any of embodiments 1-410, 452-218, 551-831, or 864-871.
887. An oligomeric compound comprising a modified oligonucleotide consisting of 12-21 linked nucleosides, wherein the modified oligonucleotide has the formula A-B-C, wherein A is a 5′-region, B is a central region, and C is a 3′-region; wherein:
   the 5′-region consists of 1-5 linked nucleosides, wherein at least one nucleoside of the 5′-region is a modified nucleoside comprising a modified sugar;
   the 3′-region consists of 1-5 linked nucleosides wherein at least one nucleoside of the 3′-region is a modified nucleoside comprising a modified sugar; and
   the central region consists of 7-11 linked nucleosides, wherein
      the 5′-most portion of the central region has the following formula:

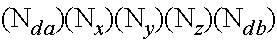

wherein one of Nₓ, N_{y}, and N_{z},is a safety-enhancing nucleoside;
the other two of Nₓ, N_{y}, and N_{z} are independently selected from a DNA nucleoside, a DNA isomer, a modified DNA isomer, and a DNA mimic; and
N_{da} and N_{db} are each independently selected from a DNA nucleoside, a DNA isomer, a modified DNA isomer, and a DNA mimic.
888. The oligomeric compound of embodiment 887, wherein the 5′-region consists of one nucleoside.
889. The oligomeric compound of embodiment 887, wherein the 5′-region consists of 2-5 linked nucleosides.
890. The oligomeric compound of embodiment 887, wherein the 5′-region consists of 2-4 linked nucleosides.
891. The oligomeric compound of embodiment 887, wherein the 5′-region consists of 2 linked nucleosides.
892. The oligomeric compound of embodiment 887, wherein the 5′-region consists of 3 linked nucleosides.
893. The oligomeric compound of embodiment 887, wherein the 5′-region consists of 4 linked nucleosides.
894. The oligomeric compound of embodiment 887, wherein the 5′-region consists of 5 linked nucleosides.
895. The oligomeric compound of any of embodiments 887-894, wherein each nucleoside of the 5′-region is a modified nucleoside.
896. The oligomeric compound of any of embodiments 887-895, wherein each nucleoside of the 5′-region is a modified nucleoside comprising a modified sugar.
897. The oligomeric compound of any of embodiments 887-896, wherein each modified nucleoside of the 5′-region has the same modification.
898. The oligomeric compound of any of embodiments 887-896, wherein at least two nucleosides of the 5′-region are modified nucleosides having different modifications.
899. The oligomeric compound of any of embodiments 887-898, wherein each nucleoside of the 5′-region comprises a 2′-modified ribosyl sugar moiety.
900. The oligomeric compound of any of embodiments 887-899, wherein at least one nucleoside of the 5′-region comprises a bicyclic furanosyl sugar moiety.
901. The oligomeric compound of any of embodiments 887-900, wherein each nucleoside of the 5′-region comprises a bicyclic furanosyl sugar moiety.
902. The oligomeric compound of any of embodiments 887-900, wherein at least one nucleoside of the 5′-region comprises a non-bicyclic sugar moiety.
903. The oligomeric compound of embodiment 902, wherein the non-bicyclic sugar moiety of the at least one nucleoside of the 5′-region is a 2′-substituted ribosyl sugar moiety.
904. The oligomeric compound of any of embodiments 887-889 or 902-903, wherein each nucleoside of the 5′-region comprises a non-bicyclic sugar moiety.
905. The oligomeric compound of embodiment 904, wherein each nucleoside of the 5′-region comprises a 2′-substituted furanosyl sugar moiety.
906. The oligomeric compound of any of embodiments 887-905, wherein each nucleoside of the 5′-region comprises a 2′-modified furanosyl sugar moiety independently selected from a bicyclic sugar moiety and a non-bicyclic 2′-substituted ribosyl sugar moiety.
907. The oligomeric compound of any of embodiments 887-901 or 906, wherein each nucleoside of the 5′-region comprises a bicyclic sugar moiety.
908. The oligomeric compound of any of embodiments 887-889 or 902-906, wherein each nucleoside of the 5′-region comprises a non-bicyclic 2′-substituted ribosyl sugar moiety.
909. The oligomeric compound of any of embodiments 887-903 or 906-907, wherein each bicyclic sugar moiety of the 5′-region is selected from among cEt, LNA, and ENA.
910. The oligomeric compound of any of embodiments 887-903 or 906-909, wherein each bicyclic sugar moiety of the 5′-region is a cEt sugar moiety.
911. The oligomeric compound of any of embodiments 887-903, or 906-907 or 909 wherein each bicyclic sugar moiety of the 5′-region is an LNA sugar moiety.
912. The oligomeric compound of any of embodiments 887-900, 902-906, or 908-911, wherein each non-bicyclic sugar moiety of the 5′-region has a 2′-substituent selected from among 2′-MOE, 2′-OMe, and 2′-NMA.
913. The oligomeric compound of any of embodiments 887-912, wherein none of the nucleosides of the 5′-region comprise a sugar moiety having a 2′-F substituent.
914. The oligomeric compound of any of embodiments 887-913, wherein none of the nucleosides of the 5′-region comprise a sugar moiety having a 2′-MOE substituent.
915. The oligomeric compound of any of embodiments 887-914, wherein none of the nucleosides of the 5′-region comprise a sugar moiety having a 2′-OMe substituent.
916. The oligomeric compound of any of embodiments 887-915, wherein none of the nucleosides of the 5′-region comprise a cEt sugar moiety.
917. The oligomeric compound of any of embodiments 887-916, wherein none of the nucleosides of the 5′-region comprise a LNA sugar moiety.
918. The oligomeric compound of any of embodiments 887-917, wherein each nucleobase of the 5′-region is independently selected from among thymine, uracil, guanine, cytosine, 5-methylcytosine, and adenine.
919. The oligomeric compound of any of embodiments 887-918, wherein each internucleoside linkage of the 5′-region is independently selected from among phosphodiester and phosphorothioate internucleoside linkages.
920. The oligomeric compound of any of embodiments 887-919, wherein the 3′-region consists of one nucleoside.
921. The oligomeric compound of any of embodiments 887-919, wherein the 3′-region consists of 2-5 linked nucleosides.
922. The oligomeric compound of any of embodiments 887-919, wherein the 3′-region consists of 2-4 linked nucleosides.
923. The oligomeric compound of any of embodiments 887-919, wherein the 3′-region consists of 2 linked nucleosides.
924. The oligomeric compound of any of embodiments 887-919, wherein the 3′-region consists of 3 linked nucleosides.
925. The oligomeric compound of any of embodiments 887-919, wherein the 3′-region consists of 4 linked nucleosides.
926. The oligomeric compound of any of embodiments 887-919, wherein the 3′-region consists of 5 linked nucleosides.
927. The oligomeric compound of any of embodiments 887-926, wherein each nucleoside of the 3′-region is a modified nucleoside.
928. The oligomeric compound of any of embodiments 887-927, wherein each nucleoside of the 3′-region is a modified nucleoside comprising a modified sugar moiety.
929. The oligomeric compound of any of embodiments 887-928, wherein each modified nucleoside of the 3′-region has the same modification.
930. The oligomeric compound of and of embodiments 887-928, wherein at least two nucleosides of the 3′-region are modified nucleosides having different modifications.
931. The oligomeric compound of any of embodiments 887-930, wherein each nucleoside of the 3′-region comprises a 2′-modified ribosyl sugar moiety.
932. The oligomeric compound of any of embodiments 887-931, wherein at least one nucleoside of the 3′-region comprises a bicyclic furanosyl sugar moiety.
933. The oligomeric compound of any of embodiments 887-932, wherein each nucleoside of the 3′-region comprises a bicyclic furanosyl sugar moiety.
934. The oligomeric compound of any of embodiments 887-933, wherein at least one nucleoside of the 3′-region comprises a non-bicyclic sugar moiety.
935. The oligomeric compound of embodiment 934, wherein the non-bicyclic sugar moiety of the at least one nucleoside of the 3′-region is a 2′-substituted ribosyl sugar moiety.
936. The oligomeric compound of any of embodiments 887-935, wherein each nucleoside of the 3′-region comprises a non-bicyclic sugar moiety.
937. The oligomeric compound of embodiment 936, wherein each nucleoside of the 3′-region comprises a 2′-substituted furanosyl sugar moiety.
938. The oligomeric compound of any of embodiments 887-937, wherein each nucleoside of the 3′-region comprises a 2′-modified furanosyl sugar moiety independently selected from a bicyclic sugar moiety and a non-bicyclic 2′-substituted ribosyl sugar moiety.
939. The oligomeric compound of any of embodiments 887-935, or 938 wherein each nucleoside of the 3′-region comprises a bicyclic sugar moiety.
940. The oligomeric compound of any of embodiments 887-932 or 934-938, wherein each nucleoside of the 3′-region comprises a non-bicyclic 2′-substituted ribosyl sugar moiety.
941. The oligomeric compound of any of embodiments 887-935 or 938-939, wherein each bicyclic sugar moiety of the 3′-region is selected from among cEt, LNA, and ENA.
942. The oligomeric compound of any of embodiments 887-935, 938-939, or 941, wherein each bicyclic sugar moiety of the 3′-region is a cEt sugar moiety.
943. The oligomeric compound of any of embodiments 887-935, 938-939, or 941, wherein each bicyclic sugar moiety of the 3′-region is an LNA sugar moiety.
944. The oligomeric compound of any of embodiments 887-932, 934-938 or 940, wherein each non-bicyclic sugar moiety of the 3′-region has a 2′-substituent selected from among 2′-MOE, 2′-OMe, and 2′-NMA.
945. The oligomeric compound of any of embodiments 887-944, wherein none of the nucleosides of the 3′-region comprise a sugar moiety having a 2′-F substituent.
946. The oligomeric compound of any of embodiments 887-945, wherein none of the nucleosides of the 3′-region comprise a sugar moiety having a 2′-MOE substituent.
947. The oligomeric compound of any of embodiments 887-946, wherein none of the nucleosides of the 3′-region comprise a sugar moiety having a 2′-OMe substituent.
948. The oligomeric compound of any of embodiments 887-947, wherein none of the nucleosides of the 3′-region comprise a cEt sugar moiety.
949. The oligomeric compound of any of embodiments 887-948, wherein none of the nucleosides of the 3′-region comprise a LNA sugar moiety.
950. The oligomeric compound of any of embodiments 887-949, wherein each nucleobase of the 3′-region is independently selected from among thymine, uracil, guanine, cytosine, 5-methylcytosine, and adenine.
951. The oligomeric compound of any of embodiments 887-950, wherein each internucleoside linkage of the 3′-region is independently selected from among phosphodiester and phosphorothioate internucleoside linkages.
952. The oligomeric compound of any of embodiments 887-951, wherein the modified nucleosides of the 5′-region have the same modifications as the modifications of the modified nucleosides of the 3′-region.
953. The oligomeric compound of any of embodiments 887-951, wherein at least one modified nucleoside of the 5′-region and one modified nucleoside of the 3′-region comprise modifications that differ from one another.
954. The oligomeric compound of any of embodiments 887-898, 900, 902-903, 906, 909-932, 934-935, 938, 941-953, wherein the 5′-region and the 3′-region together include at least one non-bicyclic 2′-substituted modified nucleoside and at least one bicyclic nucleoside.
955. The oligomeric compound of embodiment 954, where the bicyclic nucleoside is a cEt nucleoside.
956. The oligomeric compound of embodiment 954, where the bicyclic nucleoside is an LNA nucleoside.
957. The oligomeric compound of any of embodiments 954-956, wherein the non-bicyclic 2′-modified nucleoside is a 2′-MOE nucleoside.
958. The oligomeric compound of any of embodiments 954-956, wherein the non-bicyclic 2′-modified nucleoside is a 2′-OMe nucleoside.
959. The oligomeric compound of any of embodiments 954-958, wherein at least one nucleoside of the 5′-region or the 3′-region is an unmodified DNA nucleoside.
960. The oligomeric compound of any of embodiments 887-959, wherein the central region has the formula:

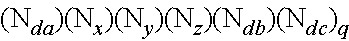

wherein each N_{dc} is independently selected from an unmodified DNA nucleoside, a DNA isomer, a modified DNA isomer, and a DNA mimic; and q is 2-6.
961. The oligomeric compound of any of embodiments 887-960, wherein the central region consists of 7 linked nucleosides.
962. The oligomeric compound of any of embodiments 887-960, wherein the central region consists of 8 linked nucleosides.
963. The oligomeric compound of any of embodiments 887-960, wherein the central region consists of 9 linked nucleosides.
964. The oligomeric compound of any of embodiments 887-960, wherein the central region consists of 10 linked nucleosides.
965. The oligomeric compound of any of embodiments 887-960, wherein the central region consists of 11 linked nucleosides.
966. The oligomeric compound of any of embodiments 887-965, wherein Nx is the safety-enhancing nucleoside.
967. The oligomeric compound of any of embodiments 887-965, wherein Ny is the safety-enhancing nucleoside.
968. The oligomeric compound of any of embodiments 887-965, wherein Nz is the safety-enhancing nucleoside.
969. The oligomeric compound of any of embodiments 887-968, wherein the safety enhancing nucleoside is a DNA isomer or a modified DNA isomer, or a modified nucleoside comprising either a sugar surrogate, a bicyclic furanosyl sugar moiety, or a non-bicyclic modified furanosyl sugar moiety.
970. The oligomeric compound of any of embodiments 887-969, wherein the safety enhancing nucleoside is a DNA isomer or a modified DNA isomer, or comprises either a sugar surrogate, a bicyclic furanosyl sugar moiety, a non-bicyclic, 2′-modified furanosyl sugar moiety, a non-bicyclic 3′-modified furanosyl sugar moiety, a non-bicyclic, 4′-modified furanosyl sugar moiety, or a non-bicyclic 5′-modified furanosyl sugar moiety.
971. The oligomeric compound of any of embodiments 887-970, wherein the safety enhancing nucleoside comprises a sugar surrogate selected from a morpholino, a modified morpholino, a modified cyclohexenyl, or a modified tetrahydropyran.
972. The oligomeric compound of any of embodiments 887-970, wherein the safety enhancing nucleoside comprises a sugar surrogate selected from a morpholino, a modified morpholino, CeNA, F-CeNA, HNA, OMe-HNA or F-HNA.
973. The oligomeric compound of any of embodiments 887-970, wherein the safety enhancing nucleoside is a DNA isomer comprising a sugar moiety selected from among 2′-β-D-deoxyxylosyl, 2′-α-L-deoxyribosyl, 2′-β-L-deoxyribosyl, 2′-α-D-deoxyxylosyl, 2′-α-D-deoxyribosyl, 2′-α-L-deoxyxylosyl, and 2′-β-L-deoxyxylosyl.
974. The oligomeric compound of any of embodiments 887-970 or 973, wherein the safety enhancing nucleoside is a DNA isomer comprising a sugar moiety selected from among 2′-β-D-deoxyxylosyl, 2′-α-L-deoxyribosyl, and 2′-β-L-deoxyribosyl.
975. The oligomeric compound of any of embodiments 887-970, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or O—C₁-C₁₀ alkoxy substituent.
976. The oligomeric compound of any of embodiments 887-970 or 975, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or OMe substituent.
977. The oligomeric compound of any of embodiments 887-970 or 975-976, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a fluoro or OMe substituent.
978. The oligomeric compound of any of embodiments 887-970 or 975-977, wherein the safety enhancing nucleoside is a modified DNA isomer comprising a sugar moiety selected from among 2′-β-D-deoxyxylosyl, 2′-α-L-deoxyribosyl, and 2′-β-L-deoxyribosyl and the 2′ position is further substituted with a hydroxyl, fluoro, or OMe substituent.
979. The oligomeric compound of any of embodiments 887-970, wherein the safety enhancing nucleoside is a modified nucleoside comprising a bicyclic furanosyl sugar moiety.
980. The oligomeric compound of any of embodiments 887-970, wherein the safety enhancing nucleoside is selected from among cEt, LNA, α-L-LNA, and ENA.
981. The oligomeric compound of any of embodiments 887-970, wherein the safety enhancing nucleoside comprises a 2′-substitued furanosyl sugar moiety.
982. The oligomeric compound of embodiment 981, wherein the 2′-substitued furanosyl sugar moiety of the safety enhancing nucleoside has a 2′ substituent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
983. The oligomeric compound of embodiment 981, wherein the 2′-substitued furanosyl sugar moiety of the safety enhancing nucleoside has a 2′ substituent selected from: halo, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
984. The oligomeric compound of embodiment 981, wherein the 2′-substitued furanosyl sugar moiety of the safety enhancing nucleoside has a 2′ substituent selected from: fluoro, OMe, MOE, NMA.
985. The oligomeric compound of any of embodiments 887-978 or 981-984, wherein the safety enhancing nucleoside comprises a 2′-substitued furanosyl sugar moiety comprising a 2′-OMe or 2′-MOE.
986. The oligomeric compound of any of embodiments 887-978 or 981-986, wherein the safety enhancing nucleoside comprises a 2′-substitued furanosyl sugar moiety comprising a 2′-OMe.
987. The oligomeric compound of any of embodiments 887-978 or 981-986, wherein the safety enhancing nucleoside comprises a 2′-OMe modified 2′-β-D-deoxyribosyl sugar moiety.
988. The oligomeric compound of any of embodiments 887-970, wherein the safety enhancing nucleoside comprises a 3′-modified furanosyl sugar moiety having a 3′ substituent selected from: halo, allyl, amino, azido, SH, CN, CF₃, OCF₃, C₁-C₁₀ alkyl, C₁-C₁₀ substituted alkyl, S-alkyl, N(Rₘ)-alkyl, N(Rₘ)-alkenyl, N(Rₘ)-alkynyl, O-alkylenyl-O-alkyl, alkynyl, alkaryl, or aralkyl.
989. The oligomeric compound of any of embodiments 887-970 or embodiment 988, wherein the safety enhancing nucleoside comprises a 3′-modified furanosyl sugar moiety having a 3′ substituent selected from: C₁-C₁₀ alkyl, or C₁-C₁₀ substituted alkyl.
990. The oligomeric compound of any of embodiments 887-970 or 988-989, wherein the safety enhancing nucleoside comprises a 3′-methyl furanosyl sugar moiety.
991. The oligomeric compound of any of embodiments 887-970, wherein the safety enhancing nucleoside comprises a 4′-modified furanosyl sugar moiety having a 4′ substituent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
992. The oligomeric compound of any of embodiments 887-970 or 991, wherein the safety enhancing nucleoside comprises a 4′-modified furanosyl sugar moiety having a 4′-methyl.
993. The oligomeric compound of any of embodiments 887-970, wherein the safety enhancing nucleoside has the structure shown below, wherein R represents an optional 2′ substituent group and Bx is a heterocyclic base moiety:

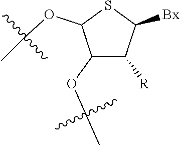

994. The oligomeric compound of embodiment 993, wherein in R is selected from among H, OH, OMe, F, or MOE.
995. The oligomeric compound of any of embodiments 887-970, wherein the safety enhancing nucleoside comprises a 5′-modified furanosyl sugar moiety having a 5′ substiuent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
996. The oligomeric compound of any of embodiments 887-970 or 995, wherein the safety enhancing nucleoside comprises a 5′-modified furanosyl sugar moiety with a 5′ substituent selected from: 5′-methyl, 5′-ethyl or a 5′-allyl.
997. The oligomeric compound of any of embodiments 887-970 or 995-996, wherein the safety enhancing nucleoside comprises a 5′-modified furanosyl sugar moiety with a 5′ substituent selected from: 5′-(R)-methyl- or 5′-(R)-ethyl.
998. The oligomeric compound of any of embodiments 887-970 or 995-997, wherein the safety enhancing nucleoside comprises a 5′-(R)-methyl-2′-β-D-deoxyribosyl sugar moiety.
999. The oligomeric compound of any of embodiments 887-970 or 995-998, wherein the safety enhancing nucleoside comprises a 5′-(R)-ethyl-2′-β-D-deoxyribosyl sugar moiety.
1000. The oligomeric compound of any of embodiments 887-999, wherein the safety enhancing nucleoside does not comprise a 2′-F modified sugar moiety.
1001. The oligomeric compound of any of embodiments 887-1000, wherein the safety enhancing nucleoside does not comprise a cEt modified sugar moiety.
1002. The oligomeric compound of any of embodiments 887-1001, wherein the safety enhancing nucleoside does not comprise a 2′-MOE modified sugar moiety.
1003. The oligomeric compound of any of embodiments 887-1002, wherein the safety enhancing nucleoside comprises a hypoxanthine nucleobase.
1004. The oligomeric compound of any of embodiments 887-1003, wherein the safety enhancing nucleoside comprises a nucleobase selected from among thymine, uracil, guanine, cytosine, 5-methylcytosine, and adenine.
1005. The oligomeric compound of any of embodiments 887-1004, wherein the safety enhancing nucleoside is a modified nucleoside other than cEt, MOE, LNA, or FANA.
1006. The oligomeric compound of any of embodiments 887-1005, wherein each Nd is independently selected from among a DNA nucleoside, a DNA isomer, a 2′-modified DNA isomer, and a DNA mimic.
1007. The oligomeric compound of embodiment 1006, wherein each DNA isomer comprises a sugar moiety selected from among 2′-β-D-deoxyxylosyl, 2′-α-L-deoxyribosyl, 2′-β-L-deoxyribosyl, 2′-α-D-deoxyxylosyl, 2′-α-D-deoxyribosyl, 2′-α-L-deoxyxylosyl, and 2′-β-L-deoxyxylosyl.
1008. The oligomeric compound of embodiment 1007, wherein each DNA isomer comprises a sugar moiety selected from among 2′-β-D-deoxyxylosyl, 2′-α-L-deoxyribosyl, and 2′-β-L-deoxyribosyl.
1009. The oligomeric compound of embodiment 1006, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or O—C₁-C₁₀alkoxy substituent.
1010. The oligomeric compound of embodiment 1009, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1, 2′-α-L-deoxyxylosyl, and 2′-β-L-deoxyxylosyl and the 2′ position is further substituted with a hydroxyl, a fluoro, or OMe substituent.
1011. The oligomeric compound of embodiment 1010, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1,2′-α-L-deoxyxylosyl, and 2′-β-L-deoxyxylosyl and the 2′ position is further substituted with a fluoro or OMe substituent.
1012. The oligomeric compound of embodiment 1011, wherein each modified DNA isomer comprises a 2′-modified sugar moiety, wherein the sugar moiety is selected from among 2′-β-D-deoxyxylosyl, 2′-α-L-deoxyribosyl, and 2′-β-L-deoxyribosyl and the 2′ position is further substituted with a hydroxyl, fluoro, or OMe substituent.
1013. The oligomeric compound of embodiment 1006, wherein each DNA mimic comprises a structure represented by one of the formulas below:

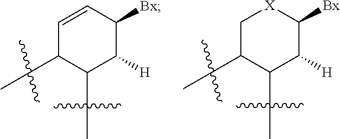

wherein X is O or S and Bx represents a heteorcylic base moiety.
1014. The oligomeric compound of embodiment 1006, wherein each DNA mimic comprises a structure represented by one of the formulas below:

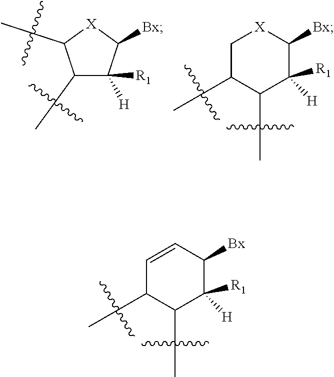

wherein X is O or S;
Bx represents a heterocyclic base moiety; and
R₁ is selected from among H, OH, halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH2C(═O)—N(Rₘ)(Rₙ), where each Rₘ and Rₙ is, independently, H, an amino protecting group, or substituted or unsubstituted C₁-C₁₀ alkyl;
wherein if the DNA mimic comprises the structure:

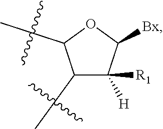

R₁ is other than H.
1015. The oligomeric compound of embodiment 1014, wherein R₁ is H, OH, OMe, or F.
1016. The oligomeric compound of embodiment 1014, wherein R₁ is not F.
1017. The oligomeric compound of embodiment 1006, wherein each DNA mimic comprises a structure represented by the formula below:

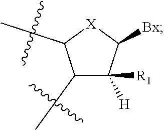

wherein X is S, Bx represents a heterocyclic base moiety, and R₁ is H.
1018. The oligomeric compound of embodiment 1006, wherein the DNA mimic has a sugar moiety selected from among: 5′-methyl-2′-β-D-deoxyribosyl, 5′-ethyl-2′-β-D-deoxyribosyl, and 5′-allyl-2′-β-D-deoxyribosyl.
1019. The oligomeric compound of embodiment 1006, wherein the DNA mimic comprises a 2′-fluoro-β-D-arabinofuranosyl sugar moiety.
1020. The oligomeric compound of embodiment 1006, wherein the DNA mimic does not comprise a 2′-fluoro-β-D-arabinofuranosyl sugar moiety.
1021. The oligomeric compound of any of embodiments 887-1020, wherein each N_{d} is a DNA nucleoside.
1022. The oligomeric compound of any of embodiments 887-1021, wherein the central region comprises no more than four nucleosides selected from among DNA isomers, modified DNA isomers, and DNA mimics.
1023. The oligomeric compound of any of embodiments 887-1021, wherein the central region comprises no more than three nucleosides selected from among DNA isomers, modified DNA isomers, and DNA mimics.
1024. The oligomeric compound of any of embodiments 887-1021, wherein the central region comprises no more than two nucleosides selected from among DNA isomers, modified DNA isomers, and DNA mimics.
1025. The oligomeric compound of any of embodiments 887-1021, wherein the central region comprises no more than one nucleoside selected from among DNA isomers, modified DNA isomers, and DNA mimics.
1026. The oligomeric compound of any of embodiments 887-1025, wherein the central region contains exactly one safety enhancing nucleoside and the remainder of nucleosides in the central region are DNA nucleosides.
1027. The oligomeric compound of any of embodiments 887-1026, wherein at least one internucleoside linkage of the central region selected from among: the internucleoside Nda to Nx, Nx to Ny, Ny to Nz, or Nz to the adjacent Ndb is a neutral internucleoside linkage.
1028. The oligomeric compound of embodiments 1027, wherein the neutral linkage is a phosphonate internucleoside linkage.
1029. The oligomeric compound of embodiments 1027, wherein the neutral linkage is a methoxypropyl internucleoside linkage.
1030. The oligomeric compound of embodiments 1027, wherein each remaining internucleoside linkage is a phosphodiester or phosphorothioate internucleoside linkage.
1031. A chirally enriched population of modified oligonucleotides of any of embodiments 887-1026, wherein the central region has at least one particular phorphorothioate internucleoside linkage having a particular stereochemical configuration.
1032. The chirally enriched population of embodiment 1031, wherein the central region has at least one phorphorothioate internucleoside linkage having the (Sp) configuration.
1033. The chirally enriched population of embodiment 1031, wherein central region has at least one phorphorothioate internucleoside linkage having the (Rp) configuration.
1034. The chirally enriched population of embodiment 1031, wherein the central region has a particular, independently selected stereochemical configuration at each phosphorothioate internucleoside linkage.
1035. The chirally enriched population of embodiment 1034, wherein the each phosphorothioate internucleoside linkage of the central region has the (Sp) configuration.
1036. The chirally enriched population of embodiment 1034, wherein the central region has one phosphorothioate internucleoside linkage having the (Rp) configuration and wherein each remaining central region phosphorothioate internucleoside linkage has the (Sp) configuration.
1037. The chirally enriched population of embodiment 1031, wherein the central region has an (Rp) phosphorothioate internucleoside linkage adjacent to an (Sp) phosphorothioate internucleoside linkage.
1038. The chirally enriched population of embodiment 1031, wherein the central region has an (Rp) phosphorothioate internucleoside linkage adjacent to a second (Rp) phosphorothioate internucleoside linkage.
1039. The chirally enriched population of embodiment 1031, wherein the central region has an (Rp) phosphorothioate internucleoside linkage adjacent to a second (Rp) phosphorothioate internucleoside linkage, and wherein each remaining central region phosphorothioate internucleoside linkage has the (Sp) configuration.
1040. The chirally enriched population of any of embodiments 1032, 1033, 1037, or 1038 wherein each phosphorothioate internucleoside linkage that does not have the (Rp) or (Sp) configuration is stereorandom.
1041. The oligomeric compound of any of embodiments 887-1040 comprising a conjugate group.
1042. The oligomeric compound of embodiment 1041, wherein the conjugate group comprises a linking group attaching the remainder of the conjugate group to the modified oligonucleotide, wherein the linking group comprises 1-5 nucleosides.
1043. The oligomeric compound of any of embodiments 887-1041, wherein the oligomeric compound does not comprise additional nucleosides beyond those of the modified oligonucleotide.
1044. The oligomeric compound of any of embodiments 887-1043, wherein the nucleobase sequence of the modified oligonucleotide is complementary to a target RNA.
1045. The oligomeric compound of embodiment 1044, wherein the nucleobase sequence of the modified oligonucleotide is at least 85% complementary to the target RNA.
1046. The oligomeric compound of embodiment 1044, wherein the nucleobase sequence of the modified oligonucleotide is at least 90% complementary to the target RNA.
1047. The oligomeric compound of embodiment 1044, wherein the nucleobase sequence of the modified oligonucleotide is at least 95% complementary to the target RNA.
1048. The oligomeric compound of embodiment 1047, wherein the nucleobase sequence of the modified oligonucleotide is 100% complementary to the target RNA.
1049. The oligomeric compound of any of embodiments 1044-1048, wherein the target RNA is a target mRNA or a target pre-mRNA.
1050. The oligomeric compound of embodiment 1049, wherein the target mRNA or target pre-mRNA is not a mouse, rat, monkey, or human PTEN RNA.
1051. The oligomeric compound of embodiment 1049 or 1050, wherein the target mRNA or target pre-mRNA is not a mouse, rat, monkey, or human SRB-1 RNA.
1052. The oligomeric compound of any of embodiments 1049-1051, wherein the target mRNA or target pre-mRNA is not a mouse, rat, monkey, or human MYD88 RNA.
1053. The oligomeric compound of any of embodiments 1049-1052, wherein the target mRNA or target pre-mRNA is not a mouse, rat, monkey, or human HTT RNA, is not a mouse, rat, monkey, or human SOD1 RNA, and is not a mouse, rat, monkey, or human alpha-synuclein RNA.
1054. The oligomeric compound of any of embodiments 1044-1053, wherein the target RNA is a human RNA.
1055. The oligomeric compound of any of embodiments 1044-1054, wherein the target RNA is expressed in the liver.
1056. The oligomeric compound of any of embodiments 1044-1055, wherein the target RNA is a liver target.
1057. The oligomeric compound of any of embodiments 1044-1054, wherein the target RNA is not expressed in the liver.
1058. The oligomeric compound of any of embodiments 1044-1054 or 1057, wherein the target RNA is not a liver target.
1059. The oligomeric compound of any of embodiments 1044-1056, wherein modulation of the expression of the target RNA in the liver is associated with treating a disorder or condition.
1060. The oligomeric compound of embodiment 1059, wherein the disorder or condition is a liver disorder or condition.
1061. The oligomeric compound of any of embodiments 1044-1060, wherein the target RNA is expressed in the central nervous system.
1062. The oligomeric compound of any of embodiments 1044-1060, wherein the target RNA is not expressed in the central nervous system.
1063. The oligomeric compound of any of embodiments 1044-1061, wherein the target RNA is a central nervous system target.
1064. The oligomeric compound of any of embodiments 1044-1062, wherein the target RNA is not a central nervous system target.
1065. The oligomeric compound of any of embodiments 1044-1064, wherein the target RNA is expressed in white fat cells.
1066. The oligomeric compound of any of embodiments 1044-1064, wherein the target RNA is expressed in brown fat cells.
1067. The oligomeric compound of any of embodiments 1044-1064, wherein the target RNA is expressed in adipocytes.
1068. The oligomeric compound of any of embodiments 1044-1064, wherein the target RNA is expressed in macrophages.
1069. The oligomeric compound of any of embodiments 1044-1064, wherein the target RNA is expressed in cancer cells.
1070. The oligomeric compound of any of embodiments 1044-1064, wherein the target RNA is expressed in tumor cells.
1071. The oligomeric compound of any of embodiments 1044-1064, wherein the target RNA is expressed in smooth muscle cells.
1072. The oligomeric compound of any of embodiments 1044-1064, wherein the target RNA is expressed in lymphocytes.
1073. The oligomeric compound of any of embodiments 1044-1064, wherein the target RNA is expressed in pulmonary cells.
1074. The oligomeric compound of any of embodiments 1044-1064, wherein the target RNA is expressed in heart muscle cells.
1075. The oligomeric compound of any of embodiments 1044-1064, wherein the target RNA is expressed in cardiomyocytes.
1076. The oligomeric compound of any of embodiments 1044-1064, wherein the target RNA is expressed in endothelial cells.
1077. The oligomeric compound of any of embodiments 1044-1064, wherein the target RNA is expressed in fibroblasts.
1078. The oligomeric compound of any of embodiments 1044-1064, wherein the target RNA is expressed in glial cells.
1079. The oligomeric compound of any of embodiments 1044-1064, wherein the target RNA is expressed in Schwann cells.
1080. The oligomeric compound of any of embodiments 1044-1064, wherein the target RNA is expressed in pancreatic cells.
1081. The oligomeric compound of any of embodiments 1044-1064, wherein the target RNA is expressed in kidney cells.
1082. The oligomeric compound of any of embodiments 1044-1064, wherein the target RNA is expressed in beta cells.
1083. The oligomeric compound of any of embodiments 1044-1064, wherein the target RNA is expressed in non-parenchymal cells.
1084. The oligomeric compound of any of embodiments 1044-1064, wherein the target RNA is expressed in hepatocytes.
1085. The oligomeric compound of any of embodiments 1044-1064, wherein the target RNA is expressed in oligodendrocytes.
1086. The oligomeric compound of any of embodiments 1044-1064, wherein the target RNA is expressed in astrocytes.
1087. The oligomeric compound of any of embodiments 1044-1064, wherein the target RNA is expressed in microglia.
1088. The oligomeric compound of any of embodiments 1044-1064, wherein the target RNA is expressed in ependymal cells.
1089. The oligomeric compound of any of embodiments 1044-1064, wherein the target RNA is expressed in sensory neurons.
1090. The oligomeric compound of any of embodiments 1044-1064, wherein the target RNA is expressed in motor neurons.
1091. The oligomeric compound of any of embodiments 1044-1064, wherein the target RNA is expressed in skeletal muscle.
1092. The oligomeric compound of any of embodiments 1044-1064, wherein the target RNA is expressed in cardiac muscle.
1093. The oligomeric compound of any of embodiments 1044-1064, wherein the target RNA is expressed in smooth muscle.
1094. The oligomeric compound of any of embodiments 1044-1064, wherein the target RNA is expressed in adipose tissue.
1095. The oligomeric compound of any of embodiments 1044-1064, wherein the target RNA is expressed in white adipose tissue.
1096. The oligomeric compound of any of embodiments 1044-1064, wherein the target RNA is expressed in the spleen.
1097. The oligomeric compound of any of embodiments 1044-1064, wherein the target RNA is expressed in the bone.
1098. The oligomeric compound of any of embodiments 1044-1064, wherein the target RNA is expressed in the bone marrow.
1099. The oligomeric compound of any of embodiments 1044-1064, wherein the target RNA is expressed in the intestine.
1100. The oligomeric compound of any of embodiments 1044-1064, wherein the target RNA is expressed in the adrenal glands.
1101. The oligomeric compound of any of embodiments 1044-1064, wherein the target RNA is expressed in the testes.
1102. The oligomeric compound of any of embodiments 1044-1064, wherein the target RNA is expressed in the ovaries.
1103. The oligomeric compound of any of embodiments 1044-1064, wherein the target RNA is expressed in the pancreas.
1104. The oligomeric compound of any of embodiments 1044-1064, wherein the target RNA is expressed in the pituitary gland.
1105. The oligomeric compound of any of embodiments 1044-1064, wherein the target RNA is expressed in the prostate gland.
1106. The oligomeric compound of any of embodiments 1044-1064, wherein the target RNA is expressed in the skin.
1107. The oligomeric compound of any of embodiments 1044-1064, wherein the target RNA is expressed in the epidermis.
1108. The oligomeric compound of any of embodiments 1044-1064, wherein the target RNA is expressed in the uterus.
1109. The oligomeric compound of any of embodiments 1044-1064, wherein the target RNA is expressed in the bladder.
1110. The oligomeric compound of any of embodiments 1044-1064, wherein the target RNA is expressed in the brain.
1111. The oligomeric compound of any of embodiments 1044-1064, wherein the target RNA is expressed in the glomerulus.
1112. The oligomeric compound of any of embodiments 1044-1064, wherein the target RNA is expressed in the distal tubular epithelium.
1113. The oligomeric compound of any of embodiments 1044-1064, wherein the target RNA is expressed in breast tissue.
1114. The oligomeric compound of any of embodiments 1044-1064, wherein the target RNA is expressed in the lung.
1115. The oligomeric compound of any of embodiments 1044-1064, wherein the target RNA is expressed in the heart.
1116. The oligomeric compound of any of embodiments 1044-1064, wherein the target RNA is expressed in the kidney.
1117. The oligomeric compound of any of embodiments 1044-1064, wherein the target RNA is expressed in the ganglion.
1118. The oligomeric compound of any of embodiments 1044-1064, wherein the target RNA is expressed in the frontal cortex.
1119. The oligomeric compound of any of embodiments 1044-1064, wherein the target RNA is expressed in the spinal cord.
1120. The oligomeric compound of any of embodiments 1044-1064, wherein the target RNA is expressed in the trigeminal ganglion.
1121. The oligomeric compound of any of embodiments 1044-1064, wherein the target RNA is expressed in the sciatic nerve.
1122. The oligomeric compound of any of embodiments 1044-1064, wherein the target RNA is expressed in the dorsal root ganglion.
1123. The oligomeric compound of any of embodiments 1044-1064, wherein the target RNA is expressed in the epidymal fat.
1124. The oligomeric compound of any of embodiments 1044-1064, wherein the target RNA is expressed in the diaphragm.
1125. The oligomeric compound of any of embodiments 1044-1064, wherein the target RNA is expressed in the colon.
1126. The oligomeric compound of any of embodiments 1044-1064, wherein the target RNA is a white fat cell target.
1127. The oligomeric compound of any of embodiments 1044-1064, wherein the target RNA is a brown fat cell target.
1128. The oligomeric compound of any of embodiments 1044-1064, wherein the target RNA is an adipocyte target.
1129. The oligomeric compound of any of embodiments 1044-1064, wherein the target RNA is a macrophage target.
1130. The oligomeric compound of any of embodiments 1044-1064, wherein the target RNA is a cancer cell target.
1131. The oligomeric compound of any of embodiments 1044-1064, wherein the target RNA is a tumor cell target.
1132. The oligomeric compound of any of embodiments 158-178, wherein the target RNA is a smooth muscle cell target.
1133. The oligomeric compound of any of embodiments 1044-1064, wherein the target RNA is a lymphocyte target.
1134. The oligomeric compound of any of embodiments 1044-1064, wherein the target RNA is a pulmonary cell target.
1135. The oligomeric compound of any of embodiments 1044-1064, wherein the target RNA is a heart muscle cell target.
1136. The oligomeric compound of any of embodiments 1044-1064, wherein the target RNA is a cardiomyocyte target.
1137. The oligomeric compound of any of embodiments 1044-1064, wherein the target RNA is a endothelial cell target.
1138. The oligomeric compound of any of embodiments 1044-1064, wherein the target RNA is a fibroblast target.
1139. The oligomeric compound of any of embodiments 1044-1064, wherein the target RNA is a glial cell target.
1140. The oligomeric compound of any of embodiments 1044-1064, wherein the target RNA is a Schwann cell target.
1141. The oligomeric compound of any of embodiments 1044-1064, wherein the target RNA is a pancreatic cell target.
1142. The oligomeric compound of any of embodiments 1044-1064, wherein the target RNA is a kidney cell target.
1143. The oligomeric compound of any of embodiments 1044-1064, wherein the target RNA is a beta cell target.
1144. The oligomeric compound of any of embodiments 1044-1064, wherein the target RNA is a non-parenchymal cell target.
1145. The oligomeric compound of any of embodiments 1044-1064, wherein the target RNA is a hepatocyte target.
1146. The oligomeric compound of any of embodiments 1044-1064, wherein the target RNA a oligodendrocyte target.
1147. The oligomeric compound of any of embodiments 1044-1064, wherein the target RNA is a astrocyte target.
1148. The oligomeric compound of any of embodiments 1044-1064, wherein the target RNA is a microglia target.
1149. The oligomeric compound of any of embodiments 1044-1064, wherein the target RNA is a ependymal cell target.
1150. The oligomeric compound of any of embodiments 1044-1064, wherein the target RNA is a sensory neuron target.
1151. The oligomeric compound of any of embodiments 1044-1064, wherein the target RNA is a motor neuron target.
1152. The oligomeric compound of any of embodiments 1044-1064, wherein the target RNA is a skeletal muscle target.
1153. The oligomeric compound of any of embodiments 1044-1064, wherein the target RNA is a cardiac muscle target.
1154. The oligomeric compound of any of embodiments 1044-1064, wherein the target RNA is a smooth muscle target.
1155. The oligomeric compound of any of embodiments 1044-1064, wherein the target RNA is a adipose tissue target.
1156. The oligomeric compound of any of embodiments 1044-1064, wherein the target RNA is a white adipose tissue target.
1157. The oligomeric compound of any of embodiments 1044-1064, wherein the target RNA is a spleen target.
1158. The oligomeric compound of any of embodiments 1044-1064, wherein the target RNA is a bone target.
1159. The oligomeric compound of any of embodiments 1044-1064, wherein the target RNA is a bone marrow target.
1160. The oligomeric compound of any of embodiments 1044-1064, wherein the target RNA is an intestinal target.
1161. The oligomeric compound of any of embodiments 1044-1064, wherein the target RNA is an adrenal gland target.
1162. The oligomeric compound of any of embodiments 1044-1064, wherein the target RNA is a testicular target.
1163. The oligomeric compound of any of embodiments 1044-1064, wherein the target RNA is an ovarian target.
1164. The oligomeric compound of any of embodiments 1044-1064, wherein the target RNA is a pancreatic target.
1165. The oligomeric compound of any of embodiments 1044-1064, wherein the target RNA is a pituitary gland target.
1166. The oligomeric compound of any of embodiments 1044-1064, wherein the target RNA is a prostate gland target.
1167. The oligomeric compound of any of embodiments 1044-1064, wherein the target RNA is a skin target.
1168. The oligomeric compound of any of embodiments 1044-1064, wherein the target RNA is an epidermal target.
1169. The oligomeric compound of any of embodiments 1044-1064, wherein the target RNA is a uterine target.
1170. The oligomeric compound of any of embodiments 1044-1064, wherein the target RNA is a bladder target.
1171. The oligomeric compound of any of embodiments 1044-1064, wherein the target RNA is a brain target.
1172. The oligomeric compound of any of embodiments 1044-1064, wherein the target RNA is a glomerulus target.
1173. The oligomeric compound of any of embodiments 1044-1064, wherein the target RNA is a distal tubular epithelium target.
1174. The oligomeric compound of any of embodiments 1044-1064, wherein the target RNA is abreast tissue target.
1175. The oligomeric compound of any of embodiments 1044-1064, wherein the target RNA is a lung target.
1176. The oligomeric compound of any of embodiments 1044-1064, wherein the target RNA is a heart target.
1177. The oligomeric compound of any of embodiments 1044-1064, wherein the target RNA is a kidney target.
1178. The oligomeric compound of any of embodiments 1044-1064, wherein the target RNA is a ganglion target.
1179. The oligomeric compound of any of embodiments 1044-1064, wherein the target RNA is a frontal cortex target.
1180. The oligomeric compound of any of embodiments 1044-1064, wherein the target RNA is a spinal cord target.
1181. The oligomeric compound of any of embodiments 1044-1064, wherein the target RNA is a trigeminal ganglion target.
1182. The oligomeric compound of any of embodiments 1044-1064, wherein the target RNA is a sciatic nerve target.
1183. The oligomeric compound of any of embodiments 1044-1064, wherein the target RNA is a dorsal root ganglion target.
1184. The oligomeric compound of any of embodiments 1044-1064, wherein the target RNA is a epidymal fat target.
1185. The oligomeric compound of any of embodiments 1044-1064, wherein the target RNA is a diaphragm target.
1186. The oligomeric compound of any of embodiments 1044-1064, wherein the target RNA is a colon target.
1187. The oligomeric compound of any of embodiments 1044-1186, wherein the target RNA is a MeCP2, DUX4, HDAC2, APP, Ataxin 1, Ataxin 2, Ataxin 3, Ataxin 6, Ataxin 7, C9ORF72, UBE3A, Prion, PMP22, Tau, LRRK2, LINGO2, GYS1, KCNT1, IRF8, Progranulin, or GFAP RNA.
1188. The oligomeric compound of any of embodiments 1044-1186, wherein the target RNA is a HTT RNA.
1189. The oligomeric compound of embodiment 1187, wherein the target RNA is a MeCP2 RNA.
1190. The oligomeric compound of embodiment 1187, wherein the target RNA is a DUX4 RNA.
1191. The oligomeric compound of embodiment 1187, wherein the target RNA is a HDAC2 RNA.
1192. The oligomeric compound of embodiment 1187, wherein the target RNA is a Ataxin 1 RNA.
1193. The oligomeric compound of embodiment 1187, wherein the target RNA is a Ataxin 2 RNA.
1194. The oligomeric compound of embodiment 1187, wherein the target RNA is a Ataxin 3 RNA.
1195. The oligomeric compound of embodiment 1187, wherein the target RNA is a Ataxin 6 RNA.
1196. The oligomeric compound of embodiment 1187, wherein the target RNA is a Ataxin 7 RNA.
1197. The oligomeric compound of embodiment 1187, wherein the target RNA is a C9ORF72 RNA.
1198. The oligomeric compound of embodiment 1044-1186, wherein the target RNA is an alpha-synuclein RNA.
1199. The oligomeric compound of embodiment 1187, wherein the target RNA is an UBE3A RNA.
1200. The oligomeric compound of any of embodiments 1044-1186, wherein the target RNA is a SOD1 RNA.
1201. The oligomeric compound of embodiment 1187, wherein the target RNA is a Prion RNA.
1202. The oligomeric compound of embodiment 1187, wherein the target RNA is a PMP22 RNA.
1203. The oligomeric compound of any of embodiments 1044-1187, wherein the target RNA is a Tau RNA.
1204. The oligomeric compound of embodiment 1187, wherein the target RNA is a LRRK2 RNA.
1205. The oligomeric compound of embodiment 1187, wherein the target RNA is an APP RNA.
1206. The oligomeric compound of 1187, wherein the target RNA is a LING02 RNA.
1207. The oligomeric compound of any of embodiments 1187, wherein the target RNA is a GYS1 RNA.
1208. The oligomeric compound of any of embodiments 1187, wherein the target RNA is a KCNT1 RNA.
1209. The oligomeric compound of any of embodiments 1187, wherein the target RNA is a IRF8 RNA.
1210. The oligomeric compound of any of embodiments 1187, wherein the target RNA is a Progranulin RNA.
1211. The oligomeric compound of any of embodiments 1187, wherein the target RNA is a GFAP RNA.
1212. The oligomeric compound of any of embodiments 1044-1211, wherein modulation of the expression of the target RNA is associated with treating a disorder or condition.
1213. The oligomeric compound of any of embodiments 1212, wherein the disorder or condition is Alzheimer's Disease, Amyotrophic Lateral Sclerosis, Parkinson's Disease, a Spinocerebellar ataxia, Angelman Syndrome, Alexander's Disease, Lafora Disease, Charcot-Marie Tooth Disease, Prion Disease, a dementia, neurodegeneration, MeCP2 Duplication Syndrome, encephalopathy, neuroinflammation, or multiple sclerosis.
1214. The oligomeric compound of embodiment 1212, wherein the disorder or condition is Alzheimer's Disease.
1215. The oligomeric compound of embodiment 1212, wherein the disorder or condition is Amyotrophic Lateral Sclerosis.
1216. The oligomeric compound of embodiment 1212, wherein the disorder or condition is Parkinson's Disease.
1217. The oligomeric compound of embodiment 1212, wherein the disorder or condition is a Spinocerebellar ataxia.
1218. The oligomeric compound of embodiment 1212, wherein the disorder or condition is Angelman Syndrome.
1219. The oligomeric compound of embodiment 1212, wherein the disorder or condition is Alexander's Disease.
1220. The oligomeric compound of embodiment 1212, wherein the disorder or condition is Lafora Disease.
1221. The oligomeric compound of embodiment 1212, wherein the disorder or condition is Charcot-Marie Tooth Disease.
1222. The oligomeric compound of embodiment 1212, wherein the disorder or condition is Prion Disease.
1223. The oligomeric compound of embodiment 1212, wherein the disorder or condition is a dementia.
1224. The oligomeric compound of embodiment 1212, wherein the disorder or condition is neurodegeneration.
1225. The oligomeric compound of embodiment 1212, wherein the disorder or condition is MeCP2 Duplication Syndrome.
1226. The oligomeric compound of embodiment 1212, wherein the disorder or condition is encephalopathy.
1227. The oligomeric compound of embodiment 1212, wherein the disorder or condition is neuroinflammation.
1228. The oligomeric compound of embodiment 1212, wherein the disorder or condition is multiple sclerosis.
1229. The oligomeric compound of any of embodiments 887-1228, wherein the parent oligomeric compound of the oligomeric compound of any of embodiments 887-1228 is cytotoxic in vitro.
1230. The oligomeric compound of embodiment 1228, wherein the parent oligomeric compound is cytotoxic in a standard in vitro cytotoxicity assay.
1231. The oligomeric compound of any of embodiments 887-1229 wherein administration to a mouse of the parent oligomeric compound of the oligomeric compound of any of embodiments 887-1229 is hepatotoxic to the mouse.
1232. The oligomeric compound of embodiment 1230, wherein the administration of the parent oligomeric compound to the mouse is systemic administration.
1233. The oligomeric compound of embodiment 1230, wherein the systemic administration is subcutaneous administration.
1234. The oligomeric compound of any of embodiments 1230-1232, wherein the mouse is a CD-1 mouse.
1235. The oligomeric compound of any of embodiments 1230-1232, wherein the mouse is a C57BL/6 mouse.
1236. The oligomeric compound of any of embodiments 1230-1232, wherein the mouse is a BALB/c mouse.
1237. The oligomeric compound of any of embodiments 1230-1236, wherein the plasma ALT level in the mouse is higher than 500 units per liter following the administration of the parent oligomeric compound.
1238. The oligomeric compound of any of embodiments 1230-1237, wherein the plasma AST level in the mouse is higher than 500 units per liter following the administration of the parent oligomeric compound.
1239. The oligomeric compound of any of embodiments 1230-1238, wherein the plasma ALT level in the mouse is higher than 1000 units per liter following the administration of the parent oligomeric compound.
1240. The oligomeric compound of any of embodiments 1230-1239, wherein the plasma AST level in the mouse is higher than 1000 units per liter following the administration of the parent oligomeric compound.
1241. The oligomeric compound of any of embodiments 1230-1240, wherein the hepatotoxicity is measured 24-48 hours following the administration of the parent oligomeric compound.
1242. The oligomeric compound of any of embodiments 1230-1241, wherein the hepatotoxicity is measured 72 hours following the administration of the parent oligomeric compound.
1243. The oligomeric compound of any of embodiments 1230-1241, wherein the hepatotoxicity is measured 3 days following the administration of the parent oligomeric compound.
1244. The oligomeric compound of any of embodiments 1230-1241, wherein the hepatotoxicity is measured 5 days following the administration of the parent oligomeric compound.
1245. The oligomeric compound of any of embodiments 1230-1241, wherein the hepatotoxicity is measured 7 days following the administration of the parent oligomeric compound.
1246. The oligomeric compound of any of embodiments 1230-1241, wherein the hepatotoxicity is measured 10 days following the administration of the parent oligomeric compound.
1247. The oligomeric compound of any of embodiments 1230-1241, wherein the hepatotoxicity is measured 14 days following the administration of the parent oligomeric compound.
1248. The oligomeric compound of any of embodiments 1230-1241, wherein 1-10 mg/kg of the parent oligomeric compound is administered to the mouse.
1249. The oligomeric compound of any of embodiments 1230-1241, wherein 10-25 mg/kg of the parent oligomeric compound is administered to the mouse.
1250. The oligomeric compound of any of embodiments 1230-1241, wherein 25-50 mg/kg of the parent oligomeric compound is administered to the mouse.
1251. The oligomeric compound of any of embodiments 1230-1241, wherein 50-100 mg/kg of the parent oligomeric compound is administered to the mouse.
1252. The oligomeric compound of any of embodiments 1230-1241, wherein 100-150 mg/kg of the parent oligomeric compound is administered to the mouse.
1253. The oligomeric compound of any of embodiments 1230-1241, wherein the mouse is a BALB/c mouse, wherein 150 mg/kg of the parent oligomeric compound is administered to the mouse, and wherein the plasma ALT level in the mouse is measured 72 hours following the administration of the parent oligomeric compound.
1254. The oligomeric compound of any of embodiments 887-1253, wherein administration of 50 mg/kg of the oligomeric compound of any of embodiments 887-1253 to a mouse is not hepatotoxic to the mouse.
1255. The oligomeric compound of embodiment 1254, wherein plasma ALT level in the mouse is lower than 100 units per liter following the administration of the oligomeric compound of embodiment 1254.
1256. The oligomeric compound of embodiment 1254 or 1255, wherein plasma ALT level in the mouse is at least 10-fold lower than plasma ALT level in a mouse administered the parent oligomeric compound of the oligomeric compound of embodiment 1254 or 1255, wherein the administrations and plasma ALT measurements of both the oligomeric compound of embodiment 1254 or 1255 and the parent oligomeric compound are completed in the same way.
1257. The oligomeric compound of embodiment 1256, wherein the plasma ALT level in the mouse is at least 100-fold lower than the plasma ALT level in the mouse administered the parent oligomeric compound.
1258. The oligomeric compound of embodiment 1256, wherein the plasma ALT level in the mouse is at least 500-fold lower than the plasma ALT level in the mouse administered the parent oligomeric compound.
1259. The oligomeric compound of any of embodiments 1230-1258, wherein the therapeutic index in a mouse of the oligomeric compound of any of embodiments 1230-1258 is increased relative to the therapeutic index of the parent oligomeric compound.
1260. The oligomeric compound of embodiment 1259, wherein the therapeutic index in a mouse of the oligomeric compound of embodiment 1259 is at least two-fold greater than the therapeutic index of the parent oligomeric compound.
1261. The oligomeric compound of any of embodiments 887-1229, wherein an otherwise identical oligomeric compound lacking the at least one modified internucleoside linkage other than phosphorothioate and/or at least one modified nucleoside comprising a modified nucleobase other than 5-methylcytosine and/or a modified sugar moiety in the central region is hepatotoxic following administration to a mouse; and wherein the otherwise identical oligomeric compound has a central region consisting of phosphorothioate internucleoside linkages, unmodified 2′-β-D-deoxyribosyl sugar moieties, and nucleobases selected from thymine, uracil, adenine, guanine, cytosine, and 5-methylcytosine.
1262. The oligomeric compound of embodiment 1261, wherein the at least one modified internucleoside linkage other than phosphorothioate and/or at least one modified nucleoside comprising a modified nucleobase other than 5-methylcytosine and/or a modified sugar moiety in the central region is a 2′-O-methyl modified sugar moiety.
1263. The oligomeric compound of embodiment 1262, wherein the 2′-O-methyl modified sugar moiety is at position 2 of the central region.
1264. The oligomeric compound of embodiment 1261-1263, wherein the otherwise identical oligomeric compound lacks a 2′-O-methyl modified sugar moiety, and wherein the otherwise identical oligomeric compound comprises an unmodified 2′-β-D-deoxyribosyl sugar moiety in place of the 2′-O-methyl modified sugar moiety.
1265. The oligomeric compound of any of embodiments 1261-1264, wherein the nucleobase sequence of the otherwise identical oligomeric compound is 100% complementary to the target RNA.
1266. The oligomeric compound of any of embodiments 1261-1265, wherein the otherwise identical oligomeric compound is a parent oligomeric compound of the oligomeric compound of any of embodiments 1202-1206.
1267. The oligomeric compound of any of embodiments 1044-1266, wherein the oligomeric compound inhibits the target RNA with an IC₅₀ lower than 100 nM in a standard in vitro activity assay.
1268. The oligomeric compound of any of embodiments 1044-1266, wherein the parent oligomeric compound of the oligomeric compound of any of embodiments 1044-1266 inhibits the target RNA with an IC₅₀ lower than 100 nM in a standard in vitro activity assay.
1269. The oligomeric compound of any of embodiments 1044-1268, wherein the difference between the IC₅₀ of the parent oligomeric compound measured in a standard in vitro activity assay and the IC₅₀ of the oligomeric compound of any of embodiments 1044-1268 measured in a standard in vitro activity assay is less than 4-fold.
1270. The oligomeric compound of any of embodiments 1044-1268, wherein the difference between the IC₅₀ of the parent oligomeric compound measured in a standard in vitro activity assay and the IC₅₀ of the oligomeric compound of any of embodiments 1044-1268 measured in a standard in vitro activity assay is less than 3-fold.
1271. The oligomeric compound of any of embodiments 1044-1268, wherein the difference between the IC₅₀ of the parent oligomeric compound measured in a standard in vitro activity assay and the IC₅₀ of the oligomeric compound of any of embodiments 1044-1268 measured in a standard in vitro activity assay is less than 2-fold.
1272. The oligomeric compound of any of embodiments 1044-1271, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
1273. The oligomeric compound of any of embodiments 1044-1272, wherein the nucleobase sequence of the parent oligomeric compound is 100% complementary to the target RNA.
1274. The oligomeric compound of any of embodiments 887-1273, wherein administration of 10-50 mg of the oligomeric compound to a human is not hepatotoxic to the human.
1275. The oligomeric compound of any of embodiments 887-1273, wherein administration of 50-100 mg of the oligomeric compound to a human is not hepatotoxic to the human.
1276. The oligomeric compound of any of embodiments 887-1273, wherein administration of 100-300 mg of the oligomeric compound to a human is not hepatotoxic to the human.
1277. The oligomeric compound of any of embodiments 1274-1276, wherein the administration is systemic administration.
1278. A composition comprising the oligomeric compound of any of embodiments 887-1277, and a second oligomeric compound, wherein the second oligomeric compound is complementary to the oligomeric compound of any of embodiments 887-1277.
1279. The composition of embodiment 1278, wherein the second oligomeric compound comprises at least 14 contiguous linked nucleosides that are at least 90% complementary to the oligomeric compound of any of embodiments 887-1277.
1280. The composition of embodiment 1278, wherein the second oligomeric compound comprises at least 14 contiguous linked nucleosides that are 100% complementary to the oligomeric compound of any of embodiments 887-1277.
1281. A pharmaceutical composition comprising the oligomeric compound of any of embodiments 887-1277 or the composition of any of embodiments 1278-1280, comprising a pharmaceutically acceptable carrier or diluent.
1282. A method comprising administering the oligomeric compound or composition of any of embodiments 887-1281 to a human subject.
1283. A method of treating a disease or disorder comprising administering the oligomeric compound or composition of any of embodiments 887-1281 to a human subject.
1284. A method of modulating the amount or activity of a target RNA in a human subject comprising administering the oligomeric compound or composition of any of embodiments 887-1281 to the human subject; and thereby modulating the amount or activity of the target RNA in the human subject.
1285. A method of treating a disease or disorder in a human subject comprising administering the oligomeric compound or composition of any of embodiments 887-1281 to the human subject having the disease or disorder; and thereby treating the disease or disorder in the human subject.
1286. The method of embodiment 1284 or 1285, wherein the disease or disorder is a liver disease, and wherein the oligomeric compound modulates the amount or activity of a liver target RNA.
1287. The method of embodiment 1284 or 1285, wherein the disease or disorder is a central nervous system disease, and wherein the oligomeric compound modulates the amount or activity of a central nervous system target RNA.
1288. The method of any of embodiments 1284-1287, wherein the disease or disorder is a cancer, a neurodegenerative disease, a cardiovascular disease, an immunological disease, a hereditary disease, a repeat expansion disease, a muscle-wasting disease, or a metabolic disease.
1289. The method of embodiment 1284 or 1285, wherein the oligomeric compound modulates the amount or activity of a white fat cell target.
1290. The method of embodiment 1284 or 1285, wherein the oligomeric compound modulates the amount or activity of a brown fat cell target.
1291. The method of embodiment 1284 or 1285, wherein the oligomeric compound modulates the amount or activity of an adipocyte target.
1292. The method of embodiment 1284 or 1285, wherein the oligomeric compound modulates the amount or activity of a macrophage target.
1293. The method of embodiment 1284 or 1285, wherein the oligomeric compound modulates the amount or activity of a cancer cell target.
1294. The method of embodiment 1284 or 1285, wherein the oligomeric compound modulates the amount or activity of a tumor cell target.
1295. The method of embodiment 1284 or 1285, wherein the oligomeric compound modulates the amount or activity of a smooth muscle cell target.
1296. The method of embodiment 1284 or 1285, wherein the oligomeric compound modulates the amount or activity of a lymphocyte target.
1297. The method of embodiment 1284 or 1285, wherein the oligomeric compound modulates the amount or activity of a pulmonary cell target.
1298. The method of embodiment 1284 or 1285, wherein the oligomeric compound modulates the amount or activity of a heart muscle cell target.
1299. The method of embodiment 1284 or 1285, wherein the oligomeric compound modulates the amount or activity of a cardiomyocyte target.
1300. The method of embodiment 1284 or 1285, wherein the oligomeric compound modulates the amount or activity of a endothelial cell target.
1301. The method of embodiment 1284 or 1285, wherein the oligomeric compound modulates the amount or activity of a fibroblast target.
1302. The method of embodiment 1284 or 1285, wherein the oligomeric compound modulates the amount or activity of a glial cell target.
1303. The method of embodiment 1284 or 1285, wherein the oligomeric compound modulates the amount or activity of a Schwann cell target.
1304. The method of embodiment 1284 or 1285, wherein the oligomeric compound modulates the amount or activity of a pancreatic cell target.
1305. The method of embodiment 1284 or 1285, wherein the oligomeric compound modulates the amount or activity of a kidney cell target.
1306. The method of embodiment 1284 or 1285, wherein the oligomeric compound modulates the amount or activity of a beta cell target.
1307. The method of embodiment 1284 or 1285, wherein the oligomeric compound modulates the amount or activity of a non-parenchymal cell target.
1308. The method of embodiment 1284 or 1285, wherein the oligomeric compound modulates the amount or activity of a hepatocyte target.
1309. The method of embodiment 1284 or 1285, wherein the oligomeric compound modulates the amount or activity of a oligodendrocyte target.
1310. The method of embodiment 1284 or 1285, wherein the oligomeric compound modulates the amount or activity of a astrocyte target.
1311. The method of embodiment 1284 or 1285, wherein the oligomeric compound modulates the amount or activity of a microglia target.
1312. The method of embodiment 1284 or 1285, wherein the oligomeric compound modulates the amount or activity of a ependymal cell target.
1313. The method of embodiment 1284 or 1285, wherein the oligomeric compound modulates the amount or activity of a sensory neuron target.
1314. The method of embodiment 1284 or 1285, wherein the oligomeric compound modulates the amount or activity of a motor neuron target.
1315. The method of embodiment 1284 or 1285, wherein the oligomeric compound modulates the amount or activity of a skeletal muscle target.
1316. The method of embodiment 1284 or 1285, wherein the oligomeric compound modulates the amount or activity of a cardiac muscle target.
1317. The method of embodiment 1284 or 1285, wherein the oligomeric compound modulates the amount or activity of a smooth muscle target.
1318. The method of embodiment 1284 or 1285, wherein the oligomeric compound modulates the amount or activity of a adipose tissue target.
1319. The method of embodiment 1284 or 1285, wherein the oligomeric compound modulates the amount or activity of a white adipose tissue target.
1320. The method of embodiment 1284 or 1285, wherein the oligomeric compound modulates the amount or activity of a spleen target.
1321. The method of embodiment 1284 or 1285, wherein the oligomeric compound modulates the amount or activity of a bone target.
1322. The method of embodiment 1284 or 1285, wherein the oligomeric compound modulates the amount or activity of a bone marrow target.
1323. The method of embodiment 1284 or 1285, wherein the oligomeric compound modulates the amount or activity of an intestinal target.
1324. The method of embodiment 1284 or 1285, wherein the oligomeric compound modulates the amount or activity of an adrenal gland target.
1325. The method of embodiment 1284 or 1285, wherein the oligomeric compound modulates the amount or activity of a testicular target.
1326. The method of embodiment 1284 or 1285, wherein the oligomeric compound modulates the amount or activity of an ovarian target.
1327. The method of embodiment 1284 or 1285, wherein the oligomeric compound modulates the amount or activity of a pancreatic target.
1328. The method of embodiment 1284 or 1285, wherein the oligomeric compound modulates the amount or activity of a pituitary gland target.
1329. The method of embodiment 1284 or 1285, wherein the oligomeric compound modulates the amount or activity of a prostate gland target.
1330. The method of embodiment 1284 or 1285, wherein the oligomeric compound modulates the amount or activity of a skin target.
1331. The method of embodiment 1284 or 1285, wherein the oligomeric compound modulates the amount or activity of an epidermal target.
1332. The method of embodiment 1284 or 1285, wherein the oligomeric compound modulates the amount or activity of a uterine target.
1333. The method of embodiment 1284 or 1285, wherein the oligomeric compound modulates the amount or activity of a bladder target.
1334. The method of embodiment 1284 or 1285, wherein the oligomeric compound modulates the amount or activity of a brain target.
1335. The method of embodiment 1284 or 1285, wherein the oligomeric compound modulates the amount or activity of a glomerulus target.
1336. The method of embodiment 1284 or 1285, wherein the oligomeric compound modulates the amount or activity of a distal tubular epithelium target.
1337. The method of embodiment 1284 or 1285, wherein the oligomeric compound modulates the amount or activity of a breast tissue target.
1338. The method of embodiment 1284 or 1285, wherein the oligomeric compound modulates the amount or activity of a lung target.
1339. The method of embodiment 1284 or 1285, wherein the oligomeric compound modulates the amount or activity of a heart target.
1340. The method of embodiment 1284 or 1285, wherein the oligomeric compound modulates the amount or activity of a kidney target.
1341. The method of embodiment 1284 or 1285, wherein the oligomeric compound modulates the amount or activity of a ganglion target.
1342. The method of embodiment 1284 or 1285, wherein the oligomeric compound modulates the amount or activity of a frontal cortex target.
1343. The method of embodiment 1284 or 1285, wherein the oligomeric compound modulates the amount or activity of a spinal cord target.
1344. The method of embodiment 1284 or 1285, wherein the oligomeric compound modulates the amount or activity of a trigeminal ganglion target.
1345. The method of embodiment 1284 or 1285, wherein the oligomeric compound modulates the amount or activity of a sciatic nerve target.
1346. The method of embodiment 1284 or 1285, wherein the oligomeric compound modulates the amount or activity of a dorsal root ganglion target.
1347. The method of embodiment 1284 or 1285, wherein the oligomeric compound modulates the amount or activity of a epidymal fat target.
1348. The method of embodiment 1284 or 1285, wherein the oligomeric compound modulates the amount or activity of a diaphragm target.
1349. The method of embodiment 1284 or 1285, wherein the oligomeric compound modulates the amount or activity of a colon target.
1350. The method of any of embodiments 1282-1349, wherein the plasma ALT level of the human subject after the administration is not more than three times higher than it was prior to the administration.
1351. The method of any of embodiments 1282-1350, wherein the plasma ALT level of the human subject after the administration is not more than 150 units per liter.
1352. The method of any of embodiments 1282-1351, wherein the human subject is susceptible to liver damage.
1353. The method of any of embodiments 1282-1352, wherein the human subject is susceptible to liver degeneration.
1354. The method of any of embodiments 1282-1353, wherein the human subject is susceptible to elevated apoptosis in the liver.
1355. The method of any of embodiments 1282-1354, wherein the human subject has a liver disease.
1356. The method of any of embodiments 1282-1355, wherein the human subject has kidney disease.
1357. The method of any of embodiments 1282-1356, wherein the human subject is susceptible to kidney damage.
1358. The method of any of embodiments 1282-1357, wherein the human subject has heart disease.
1359. The method of any of embodiments 1282-1358, wherein the human subject is susceptible to heart damage.
1360. The method of any of embodiments 1282-1359, wherein the human subject has pancreatitis.
1361. The method of any of embodiments 1282-1360, wherein the human subject is susceptible to pancreatic damage.
1362. The method of any of embodiments 1282-1361, wherein the human subject has a neurological disease.
1363. The method of any of embodiments 1282-1362, wherein the human subject is susceptible to neurological damage.
1364. The method of any of embodiments 1282-1363, comprising administering the parent oligomeric compound of the oligomeric compound of any of embodiments 887-1281 to a mouse.
1365. The method of any of embodiments 1282-1363, comprising contacting a cell with the parent oligomeric compound of the oligomeric compound of any of embodiments 887-1281.
1366. The method of embodiment 1364 or 1365, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
1367. A method of designing an oligomeric compound comprising starting with a parent oligomeric compound and changing the design of that compound in order to arrive at an oligomeric compound of any one of embodiments 887-1281.
1368. A method of designing an oligomeric compound comprising identifying a parent oligomeric compound and changing the design of that parent oligomeric compound to arrive at a second oligomeric compound, wherein the second oligomeric compound is an oligomeric compound of any one of embodiments 887-1281.
1369. A method of improving hepatotoxicity of an oligomeric compound comprising the steps of (i) identifying a parent oligomeric compound that has plasma ALT levels above 300 units per liter in a mouse, and (ii) providing an oligomeric compound according to any one of embodiments 887-1281.
1370. The method of embodiment 1368 or 1369, wherein the method designs an oligomeric compound with improved therapeutic index relative to the parent oligomeric compound.
1371. The method of embodiment 1368 or 1369, wherein the method designs an oligomeric compound with lower hepatotoxicity relative to the parent oligomeric compound.
1372. The method of embodiment 1368, wherein the second oligomeric compound has an improved therapeutic index relative to the parent oligomeric compound.
1373. The method of embodiment 1368 or 1369, wherein the second oligomeric compound has reduced hepatotoxicity in a mouse relative to the parent oligomeric compound.
1374. The method of embodiment 1368, wherein the oligomeric compound according to any one of embodiments 887-1281 has reduced hepatotoxicity relative to the parent oligomeric compound.
1375. A method comprising administering an oligomeric compound of any of embodiments 887-1281 to a mouse and separately administering the parent oligomeric compound of the oligomeric compound of any of embodiments 887-1281 to a second mouse, wherein the therapeutic index of the oligomeric compound of any of embodiments 887-1281 is improved relative to the therapeutic index of the parent oligomeric compound.
1376. The method of any of embodiments 1282-1375, wherein the level of p21 RNA is measured in the mouse, the cell, and/or the human subject.
1377. A method comprising administering an oligomeric compound of any of embodiments 887-1281 to a subject and measuring the level of p21 RNA in the subject.
1378. The method of embodiment 1377, wherein the subject is a mouse.
1379. The method of embodiment 1377, wherein the subject is a human.
1380. The method of any of embodiments 1377-1379, wherein the p21 RNA level is measured within 24 hours of the administration.
1381. The method of any of embodiments 1377-1380, wherein the p21 RNA level is measured 24-48 hours following the administration.
1382. An oligomeric compound or composition of any one of embodiments 887-1281, for use in medical therapy.
1383. The oligomeric compound of any of embodiments 887-1281, wherein the oligomeric compound is not toxic.
1384. The oligomeric compound of any of embodiment 887-1281, wherein a comparator compound is toxic; wherein the comparator compound is identical to the oligomeric compound except that each nucleoside of the central region of the comparator compound is a nucleoside comprising 2′-β-D-deoxyribosyl sugar moiety.
1385. The oligomeric compound of embodiment 1384, wherein administering a dose of the oligomeric compound to an animal results in a circulating ALT level that is at least 3-fold lower than the ALT level that results from administering the same dose of the comparator compound to an animal for at least one matched dose.
1386. The oligomeric compound of embodiment 1384 or 1385, wherein the lowest dose of the oligomeric compound that raises circulating ALT to over 300 units per liter is at least three-fold higher than the lowest dose of the comparator compound that raises circulating ALT to over 300 units per liter.
1387. The oligomeric compound of any of embodiments 1384-1386, wherein the oligomeric compound is capable of reducing the target RNA in a cell.
1388. The oligomeric compound of embodiment 1387, wherein the oligomeric compound has RNA-reducing activity comparable to a comparator compound, wherein the comparator compound is identical to the oligomeric compound except that each nucleoside of the central region of the comparator compound is a nucleoside comprising 2′-β-D-deoxyribosyl sugar moiety.
1389. The oligomeric compound of embodiment 1388 having RNA-reducing activity that is no more than 2-fold worse than the RNA-reducing activity of the comparator compound.
1390. A method of modulating the amount or activity of a target RNA in a human subject comprising administering the oligomeric compound or composition of any of embodiments 887-1281 to the human subject; and thereby modulating the amount or activity of the target RNA in the human subject.
1391. A method of treating a disease or disorder in a human subject comprising administering the oligomeric compound or composition of any of embodiments 887-1281 to the human subject having the disease or disorder; and thereby treating the disease or disorder in the human subject.
1392. The method of embodiment 1390 or 1391, wherein the disease or disorder is a liver disease, and wherein the oligomeric compound modulates the amount or activity of a liver target RNA.
1393. The method of embodiment 1390 or 1391, wherein the disease or disorder is a central nervous system disease, and wherein the oligomeric compound modulates the amount or activity of a central nervous system target RNA.
1394. The method of any of embodiments 1390-1393, wherein the disease or disorder is a cancer, a neurodegenerative disease, a cardiovascular disease, an immunological disease, a hereditary disease, a repeat expansion disease, a muscle-wasting disease, or a metabolic disease.
1395. The method of any of embodiments 1390-1394, wherein the plasma ALT level of the human subject after the administration is not more than three times higher than it was prior to the administration.
1396. The method of any of embodiments 1390-1395, wherein the plasma ALT level of the human subject after the administration is not more than 150 units per liter.
1397. A method of treating a disease or disorder in a human subject comprising administering the oligomeric compound or composition of any of embodiments 887-1281 to the human subject having the disease or disorder; and thereby treating the disease or disorder in the human subject.
1398. The method of embodiment 1397, wherein the disease or disorder is not a CNS disease or disorder.
1399. The method of embodiment 1397, wherein the disease or disorder is a disease or disorder of the white fat cells.
1400. The method of embodiment 1397, wherein the disease or disorder is a disease or disorder of the brown fat cells.
1401. The method of embodiment 1397, wherein the disease or disorder is a disease or disorder of the adipocytes.
1402. The method of embodiment 1397, wherein the disease or disorder is a disease or disorder of the macrophages.
1403. The method of embodiment 1397, wherein the disease or disorder is a disease or disorder of the cancer cells.
1404. The method of embodiment 1397, wherein the disease or disorder is a disease or disorder of the tumor cells.
1405. The method of embodiment 1397, wherein the disease or disorder is a disease or disorder of the smooth muscle cells.
1406. The method of embodiment 1397, wherein the disease or disorder is a disease or disorder of the lymphocytes.
1407. The method of embodiment 1397, wherein the disease or disorder is a disease or disorder of the pulmonary cells.
1408. The method of embodiment 1397, wherein the disease or disorder is a disease or disorder of the heart muscle cells.
1409. The method of embodiment 1397, wherein the disease or disorder is a disease or disorder of the cardiomyocytes.
1410. The method of embodiment 1397, wherein the disease or disorder is a disease or disorder of the endothelial cells.
1411. The method of embodiment 1397, wherein the disease or disorder is a disease or disorder of the fibroblasts.
1412. The method of embodiment 1397, wherein the disease or disorder is a disease or disorder of the glial cells.
1413. The method of embodiment 1397, wherein the disease or disorder is a disease or disorder of the Schwann cells.
1414. The method of embodiment 1397, wherein the disease or disorder is a disease or disorder of the pancreatic cells.
1415. The method of embodiment 1397, wherein the disease or disorder is a disease or disorder of the kidney cells.
1416. The method of embodiment 1397, wherein the disease or disorder is a disease or disorder of the beta cells.
1417. The method of embodiment 1397, wherein the disease or disorder is a disease or disorder of the non-parenchymal cells.
1418. The method of embodiment 1397, wherein the disease or disorder is a disease or disorder of the hepatocytes.
1419. The method of embodiment 1397, wherein the disease or disorder is a disease or disorder of the oligodendrocytes.
1420. The method of embodiment 1397, wherein the disease or disorder is a disease or disorder of the astrocytes.
1421. The method of embodiment 1397, wherein the disease or disorder is a disease or disorder of the microglia.
1422. The method of embodiment 1397, wherein the disease or disorder is a disease or disorder of the ependymal cells.
1423. The method of embodiment 1397, wherein the disease or disorder is a disease or disorder of the sensory neurons.
1424. The method of embodiment 1397, wherein the disease or disorder is a disease or disorder of the motor neurons.
1425. The method of embodiment 1397, wherein the disease or disorder is a disease or disorder of the skeletal muscle.
1426. The method of embodiment 1397, wherein the disease or disorder is a disease or disorder of the cardiac muscle.
1427. The method of embodiment 1397, wherein the disease or disorder is a disease or disorder of the smooth muscle.
1428. The method of embodiment 1397, wherein the disease or disorder is a disease or disorder of the adipose tissue.
1429. The method of embodiment 1397, wherein the disease or disorder is a disease or disorder of the white adipose tissue.
1430. The method of embodiment 1397, wherein the disease or disorder is a disease or disorder of the spleen.
1431. The method of embodiment 1397, wherein the disease or disorder is a disease or disorder of the bone.
1432. The method of embodiment 1397, wherein the disease or disorder is a disease or disorder of the bone marrow.
1433. The method of embodiment 1397, wherein the disease or disorder is a disease or disorder of the intestine.
1434. The method of embodiment 1397, wherein the disease or disorder is a disease or disorder of the adrenal glands.
1435. The method of embodiment 1397, wherein the disease or disorder is a disease or disorder of the testes.
1436. The method of embodiment 1397, wherein the disease or disorder is a disease or disorder of the ovaries.
1437. The method of embodiment 1397, wherein the disease or disorder is a disease or disorder of the pancreas.
1438. The method of embodiment 1397, wherein the disease or disorder is a disease or disorder of the pituitary gland.
1439. The method of embodiment 1397, wherein the disease or disorder is a disease or disorder of the prostate gland.
1440. The method of embodiment 1397, wherein the disease or disorder is a disease or disorder of the skin.
1441. The method of embodiment 1397, wherein the disease or disorder is a disease or disorder of the epidermis.
1442. The method of embodiment 1397, wherein the disease or disorder is a disease or disorder of the uterus.
1443. The method of embodiment 1397, wherein the disease or disorder is a disease or disorder of the bladder.
1444. The method of embodiment 1397, wherein the disease or disorder is a disease or disorder of the brain.
1445. The method of embodiment 1397, wherein the disease or disorder is a disease or disorder of the glomerulus.
1446. The method of embodiment 1397, wherein the disease or disorder is a disease or disorder of the distal tubular epithelium.
1447. The method of embodiment 1397, wherein the disease or disorder is a disease or disorder of the breast tissue.
1448. The method of embodiment 1397, wherein the disease or disorder is a disease or disorder of the lung.
1449. The method of embodiment 1397, wherein the disease or disorder is a disease or disorder of the heart.
1450. The method of embodiment 1397, wherein the disease or disorder is a disease or disorder of the kidney.
1451. The method of embodiment 1397, wherein the disease or disorder is a disease or disorder of the ganglion.
1452. The method of embodiment 1397, wherein the disease or disorder is a disease or disorder of the frontal cortex.
1453. The method of embodiment 1397, wherein the disease or disorder is a disease or disorder of the spinal cord.
1454. The method of embodiment 1397, wherein the disease or disorder is a disease or disorder of the trigeminal ganglion.
1455. The method of embodiment 1397, wherein the disease or disorder is a disease or disorder of the sciatic nerve.
1456. The method of embodiment 1397, wherein the disease or disorder is a disease or disorder of the dorsal root ganglion.
1457. The method of embodiment 1397, wherein the disease or disorder is a disease or disorder of the epidymal fat.
1458. The method of embodiment 1397, wherein the disease or disorder is a disease or disorder of the diaphragm.
1459. The method of embodiment 1397, wherein the disease or disorder is a disease or disorder of the colon.
1460. A method of screening a library of oligomeric compounds for activity against a target RNA, wherein the library of oligomeric compounds comprises a plurality of oligomeric compounds of any of embodiments 887-1281.
1461. An oligomeric compound comprising a modified oligonucleotide consisting of 12-21 linked nucleosides, wherein the modified oligonucleotide has the formula A-B-C, wherein A is a 5′-region, B is a central region, and C is a 3′-region; wherein:
   the 5′-region consists of 1-5 linked nucleosides, wherein at least one nucleoside of the 5′-region is a modified nucleoside comprising a modified sugar;
   the 3′-region consists of 1-5 linked nucleosides wherein at least one nucleoside of the 3′-region is a modified nucleoside comprising a modified sugar; and
   the central region consists of 7-11 linked nucleosides, wherein
      the 5′-most portion of the central region has the following formula:

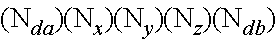

wherein one of Nₓ, N_{y}, and N_{z},is a safety-enhancing nucleoside;
the other two of Nₓ, N_{y}, and N_{z} are independently selected from an unmodified 2′-β-D-deoxyribosyl sugar moiety, a DNA isomer, a modified DNA isomer, and a DNA mimic; and
N_{da} and N_{db} are each independently selected from an unmodified 2′-β-D-deoxyribosyl sugar moiety, a DNA isomer, a modified DNA isomer, and a DNA mimic.
1462. The oligomeric compound of embodiment 1461, wherein the 5′-region consists of one nucleoside.
1463. The oligomeric compound of embodiment 1461, wherein the 5′-region consists of 2-5 linked nucleosides.
1464. The oligomeric compound of embodiment 1461, wherein the 5′-region consists of 2-4 linked nucleosides.
1465. The oligomeric compound of embodiment 1461, wherein the 5′-region consists of 2 linked nucleosides.
1466. The oligomeric compound of embodiment 1461, wherein the 5′-region consists of 3 linked nucleosides.
1467. The oligomeric compound of embodiment 1461, wherein the 5′-region consists of 4 linked nucleosides.
1468. The oligomeric compound of embodiment 1461, wherein the 5′-region consists of 5 linked nucleosides.
1469. The oligomeric compound of any of embodiments 1461-1468, wherein each nucleoside of the 5′-region is a modified nucleoside.
1470. The oligomeric compound of any of embodiments 1461-1469, wherein each nucleoside of the 5′-region is a modified nucleoside comprising a modified sugar.
1471. The oligomeric compound of any of embodiments 1461-1470, wherein each modified nucleoside of the 5′-region has the same modification.
1472. The oligomeric compound of and of embodiments 1461-1470, wherein at least two nucleosides of the 5′-region are modified nucleosides having different modifications.
1473. The oligomeric compound of any of embodiments 1461-1472, wherein each nucleoside of the 5′-region comprises a 2′-modified ribosyl sugar moiety.
1474. The oligomeric compound of any of embodiments 1461-1473, wherein at least one nucleoside of the 5′-region comprises a bicyclic furanosyl sugar moiety.
1475. The oligomeric compound of any of embodiments 1461-1474, wherein each nucleoside of the 5′-region comprises a bicyclic furanosyl sugar moiety.
1476. The oligomeric compound of any of embodiments 1461-1474, wherein at least one nucleoside of the 5′-region comprises a non-bicyclic sugar moiety.
1477. The oligomeric compound of embodiment 1476, wherein the non-bicyclic sugar moiety of the at least one nucleoside of the 5′-region is a 2′-substituted ribosyl sugar moiety.
1478. The oligomeric compound of any of embodiments 1461-1474 or 1476-1477, wherein each nucleoside of the 5′-region comprises a non-bicyclic sugar moiety.
1479. The oligomeric compound of embodiment 1478, wherein each nucleoside of the 5′-region comprises a 2′-substituted furanosyl sugar moiety.
1480. The oligomeric compound of any of embodiments 1461-1474 or 1476-1477, wherein each nucleoside of the 5′-region comprises a 2′-modified furanosyl sugar moiety independently selected from a bicyclic sugar moiety and a non-bicyclic 2′-substituted ribosyl sugar moiety.
1481. The oligomeric compound of any of embodiments 1461-1477 or 1480, wherein each nucleoside of the 5′-region comprises a bicyclic sugar moiety.
1482. The oligomeric compound of any of embodiments 1461-1474 or 1476-1480, wherein each nucleoside of the 5′-region comprises a non-bicyclic 2′-substituted ribosyl sugar moiety.
1483. The oligomeric compound of any of embodiments 1461-1477 or 1480-1481, wherein each bicyclic sugar moiety of the 5′-region is selected from among cEt, LNA, and ENA.
1484. The oligomeric compound of any of embodiments 1461-1477 or 1480-1481, wherein each bicyclic sugar moiety of the 5′-region is a cEt sugar moiety.
1485. The oligomeric compound of any of embodiments 1461-1477 or 1480-1481, wherein each bicyclic sugar moiety of the 5′-region is an LNA sugar moiety.
1486. The oligomeric compound of any of embodiments 1461-1474, 1476-1480, or 1482-1485, wherein each non-bicyclic sugar moiety of the 5′-region has a 2′-substituent selected from among 2′-MOE, 2′-OMe, and 2′-NMA.
1487. The oligomeric compound of any of embodiments 1461-1486, wherein none of the nucleosides of the 5′-region comprise a sugar moiety having a 2′-F substituent.
1488. The oligomeric compound of any of embodiments 1461-1487, wherein none of the nucleosides of the 5′-region comprise a sugar moiety having a 2′-MOE substituent.
1489. The oligomeric compound of any of embodiments 1461-1488, wherein none of the nucleosides of the 5′-region comprise a sugar moiety having a 2′-OMe substituent.
1490. The oligomeric compound of any of embodiments 1461-1489, wherein none of the nucleosides of the 5′-region comprise a cEt sugar moiety.
1491. The oligomeric compound of any of embodiments 1461-1490, wherein none of the nucleosides of the 5′-region comprise a LNA sugar moiety.
1492. The oligomeric compound of any of embodiments 1461-1491, wherein each nucleobase of the 5′-region is independently selected from among thymine, uracil, guanine, cytosine, 5-methylcytosine, and adenine.
1493. The oligomeric compound of any of embodiments 1461-1492, wherein each internucleoside linkage of the 5′-region is independently selected from among phosphodiester and phosphorothioate internucleoside linkages.
1494. The oligomeric compound of any of embodiments 1461-1493, wherein the 3′-region consists of one nucleoside.
1495. The oligomeric compound of any of embodiments 1461-1493, wherein the 3′-region consists of 2-5 linked nucleosides.
1496. The oligomeric compound of any of embodiments 1461-1493, wherein the 3′-region consists of 2-4 linked nucleosides.
1497. The oligomeric compound of any of embodiments 1461-1493, wherein the 3′-region consists of 2 linked nucleosides.
1498. The oligomeric compound of any of embodiments 1461-1493, wherein the 3′-region consists of 3 linked nucleosides.
1499. The oligomeric compound of any of embodiments 1461-1493, wherein the 3′-region consists of 4 linked nucleosides.
1500. The oligomeric compound of any of embodiments 1461-1493, wherein the 3′-region consists of 5 linked nucleosides.
1501. The oligomeric compound of any of embodiments 1461-1500, wherein each nucleoside of the 3′-region is a modified nucleoside.
1502. The oligomeric compound of any of embodiments 1461-1501, wherein each nucleoside of the 3′-region is a modified nucleoside comprising a modified sugar moiety.
1503. The oligomeric compound of any of embodiments 1461-1502, wherein each modified nucleoside of the 3′-region has the same modification.
1504. The oligomeric compound of and of embodiments 1461-1502, wherein at least two nucleosides of the 3′-region are modified nucleosides having different modifications.
1505. The oligomeric compound of any of embodiments 1461-1504, wherein each nucleoside of the 3′-region comprises a 2′-modified ribosyl sugar moiety.
1506. The oligomeric compound of any of embodiments 1461-1505, wherein at least one nucleoside of the 3′-region comprises a bicyclic furanosyl sugar moiety.
1507. The oligomeric compound of any of embodiments 1461-1506, wherein each nucleoside of the 3′-region comprises a bicyclic furanosyl sugar moiety.
1508. The oligomeric compound of any of embodiments 1461-1506, wherein at least one nucleoside of the 3′-region comprises a non-bicyclic sugar moiety.
1509. The oligomeric compound of embodiment 1508, wherein the non-bicyclic sugar moiety of the at least one nucleoside of the 3′-region is a 2′-substituted ribosyl sugar moiety.
1510. The oligomeric compound of any of embodiments 1461-1509, wherein each nucleoside of the 3′-region comprises a non-bicyclic sugar moiety.
1511. The oligomeric compound of embodiment 1510, wherein each nucleoside of the 3′-region comprises a 2′-substituted furanosyl sugar moiety.
1512. The oligomeric compound of any of embodiments 1461-1511, wherein each nucleoside of the 3′-region comprises a 2′-modified furanosyl sugar moiety independently selected from a bicyclic sugar moiety and a non-bicyclic 2′-substituted ribosyl sugar moiety.
1513. The oligomeric compound of any of embodiments 1461-1512, wherein each nucleoside of the 3′-region comprises a bicyclic sugar moiety.
1514. The oligomeric compound of any of embodiments 1461-1512, wherein each nucleoside of the 3′-region comprises a non-bicyclic 2′-substituted ribosyl sugar moiety.
1515. The oligomeric compound of any of embodiments 1461-1509 or 1512-1513, wherein each bicyclic sugar moiety of the 3′-region is selected from among cEt, LNA, and ENA.
1516. The oligomeric compound of any of embodiments 1461-1509, 1512-1513, or 1515, wherein each bicyclic sugar moiety of the 3′-region is a cEt sugar moiety.
1517. The oligomeric compound of any of embodiments 1461-1509, 1512-1513, or 1515, wherein each bicyclic sugar moiety of the 3′-region is an LNA sugar moiety.
1518. The oligomeric compound of any of embodiments 1461-1506, 1508-1512 or 1514, wherein each non-bicyclic sugar moiety of the 3′-region has a 2′-substituent selected from among 2′-MOE, 2′-OMe, and 2′-NMA.
1519. The oligomeric compound of any of embodiments 1461-1518, wherein none of the nucleosides of the 3′-region comprise a sugar moiety having a 2′-F substituent.
1520. The oligomeric compound of any of embodiments 1461-1519, wherein none of the nucleosides of the 3′-region comprise a sugar moiety having a 2′-MOE substituent.
1521. The oligomeric compound of any of embodiments 1461-1520, wherein none of the nucleosides of the 3′-region comprise a sugar moiety having a 2′-OMe substituent.
1522. The oligomeric compound of any of embodiments 1461-1521, wherein none of the nucleosides of the 3′-region comprise a cEt sugar moiety.
1523. The oligomeric compound of any of embodiments 1461-1522, wherein none of the nucleosides of the 3′-region comprise a LNA sugar moiety.
1524. The oligomeric compound of any of embodiments 1461-1523, wherein each nucleobase of the 3′-region is independently selected from among thymine, uracil, guanine, cytosine, 5-methylcytosine, and adenine.
1525. The oligomeric compound of any of embodiments 1461-1524, wherein each internucleoside linkage of the 3′-region is independently selected from among phosphodiester and phosphorothioate internucleoside linkages.
1526. The oligomeric compound of any of embodiments 1461-1525, wherein the modified nucleosides of the 5′-region have the same modifications as the modifications of the modified nucleosides of the 3′-region.
1527. The oligomeric compound of any of embodiments 1461-1525, wherein at least one modified nucleoside of the 5′-region and one modified nucleoside of the 3′-region comprise modifications that differ from one another.
1528. The oligomeric compound of any of embodiments 1461-1474, 1476-1477, 1480, 1483-1506, 1508-1509, 1512, 1515-1527, wherein the 5′-region and the 3′-region together include at least one non-bicyclic 2′-substituted modified nucleoside and at least one bicyclic nucleoside.
1529. The oligomeric compound of any of embodiment 1528, where the bicyclic nucleoside is a cEt nucleoside.
1530. The oligomeric compound of embodiment 1528, where the bicyclic nucleoside is an LNA nucleoside.
1531. The oligomeric compound of any of embodiments 1528-1530, wherein the non-bicyclic 2′-modified nucleoside is a 2′-MOE nucleoside.
1532. The oligomeric compound of any of embodiments 1528-1530, wherein the non-bicyclic 2′-modified nucleoside is a 2′-OMe nucleoside.
1533. The oligomeric compound of any of embodiments 1528-1532, wherein at least one nucleoside of the 5′-region or the 3′-region is an unmodified 2′-β-D-deoxyribosyl sugar moiety.
1534. The oligomeric compound of any of embodiments 1461-1533, wherein the central region has the formula:

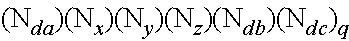

wherein each N_{dc} is independently selected from an unmodified 2′-β-D-deoxyribosyl sugar moiety, a DNA isomer, a modified DNA isomer, and a DNA mimic; and q is 2-6.
1535. The oligomeric compound of any of embodiments 1461-1534, wherein the central region consists of 7 linked nucleosides.
1536. The oligomeric compound of any of embodiments 1461-1534, wherein the central region consists of 8 linked nucleosides.
1537. The oligomeric compound of any of embodiments 1461-1534, wherein the central region consists of 9 linked nucleosides.
1538. The oligomeric compound of any of embodiments 1461-1534, wherein the central region consists of 10 linked nucleosides.
1539. The oligomeric compound of any of embodiments 1461-1534, wherein the central region consists of 11 linked nucleosides.
1540. The oligomeric compound of any of embodiments 1461-1539, wherein Nx is the safety-enhancing nucleoside.
1541. The oligomeric compound of any of embodiments 1461-1539, wherein Ny is the safety-enhancing nucleoside.
1542. The oligomeric compound of any of embodiments 1461-1539, wherein Nz is the safety-enhancing nucleoside.
1543. The oligomeric compound of any of embodiments 1461-1542, wherein the safety enhancing nucleoside is a DNA isomer or a modified DNA isomer, or a modified nucleoside comprising either a sugar surrogate, a bicyclic furanosyl sugar moiety, or a non-bicyclic modified furanosyl sugar moiety.
1544. The oligomeric compound of any of embodiments 1461-1543, wherein the safety enhancing nucleoside is a DNA isomer or a modified DNA isomer, or comprises either a sugar surrogate, a bicyclic furanosyl sugar moiety, a non-bicyclic, 2′-modified furanosyl sugar moiety, a non-bicyclic 3′-modified furanosyl sugar moiety, a non-bicyclic, 4′-modified furanosyl sugar moiety, or a non-bicyclic 5′-modified furanosyl sugar moiety.
1545. The oligomeric compound of any of embodiments 1461-1544, wherein the safety enhancing nucleoside comprises a sugar surrogate selected from a morpholino, a modified morpholino, a modified cyclohexenyl, or a modified tetrahydropyran.
1546. The oligomeric compound of any of embodiments 1461-1544, wherein the safety enhancing nucleoside comprises a sugar surrogate selected from a morpholino, a modified morpholino, 2′-fluoroarabinose, 2′-fluororibose, CeNA, F-CeNA, HNA, OMe-HNA or F-HNA.
1547. The oligomeric compound of any of embodiments 1461-1544, wherein the safety enhancing nucleoside is a DNA isomer comprising a sugar moiety selected from among 2′-β-D-deoxyxylosyl, 2′-α-L-deoxyribosyl, 2′-β-L-deoxyribosyl, 2′-α-D-deoxyxylosyl, 2′-α-D-deoxyribosyl, 2′-α-L-deoxyxylosyl, and 2′-β-L-deoxyxylosyl.
1548. The oligomeric compound of any of embodiments 1461-1544 or 1547, wherein the safety enhancing nucleoside is a DNA isomer comprising a sugar moiety selected from among 2′-β-D-deoxyxylosyl, 2′-α-L-deoxyribosyl, and 2′-β-L-deoxyribosyl.
1549. The oligomeric compound of any of embodiments 1461-1544, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1,2′-α-L-deoxyxylosyl, and 2′-β-L-deoxyxylosyl and the 2′ position is further substituted with a hydroxyl, a fluoro, or O—C₁-C₁₀ alkoxy substituent.
1550. The oligomeric compound of any of embodiments 1461-1544 or 1549, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1,2′-α-L-deoxyxylosyl, and 2′-β-L-deoxyxylosyl and the 2′ position is further substituted with a hydroxyl, a fluoro, or OMe substituent.
1551. The oligomeric compound of any of embodiments 1461-1544 or 1549-1550, wherein the safety enhancing nucleoside is a modified DNA isomer comprising a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1,2′-α-L-deoxyxylosyl, and 2′-β-L-deoxyxylosyl and the 2′ position is further substituted with a fluoro or OMe substituent.
1552. The oligomeric compound of any of embodiments 1461-1544 or 1549-1551, wherein the safety enhancing nucleoside is a modified DNA isomer comprising a sugar moiety selected from among 2′-β-D-deoxyxylosyl, 2′-α-L-deoxyribosyl, and 2′-β-L-deoxyribosyl and the 2′ position is further substituted with a hydroxyl, fluoro, or OMe substituent.
1553. The oligomeric compound of any of embodiments 1461-1544, wherein the safety enhancing nucleoside is a modified nucleoside comprising a bicyclic furanosyl sugar moiety.
1554. The oligomeric compound of any of embodiments 1461-1544, wherein the safety enhancing nucleoside is selected from among cEt, LNA, α-L-LNA, and ENA.
1555. The oligomeric compound of any of embodiments 1461-1544, wherein the safety enhancing nucleoside comprises a 2′-substitued furanosyl sugar moiety.
1556. The oligomeric compound of embodiment 1555, wherein the 2′-substitued furanosyl sugar moiety of the safety enhancing nucleoside has a 2′ substituent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
1557. The oligomeric compound of embodiment 1555, wherein the 2′-substitued furanosyl sugar moiety of the safety enhancing nucleoside has a 2′ substituent selected from: halo, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
1558. The oligomeric compound of embodiment 1555, wherein the 2′-substitued furanosyl sugar moiety of the safety enhancing nucleoside has a 2′ substituent selected from: fluoro, OMe, MOE, NMA.
1559. The oligomeric compound of any of embodiments 1461-1558, wherein the safety enhancing nucleoside comprises a 2′-substitued furanosyl sugar moiety comprising a 2′-OMe or 2′-MOE.
1560. The oligomeric compound of any of embodiments 1461-1559, wherein the safety enhancing nucleoside comprises a 2′-substitued furanosyl sugar moiety comprising a 2′-OMe.
1561. The oligomeric compound of any of embodiments 1461-1560, wherein the safety enhancing nucleoside comprises a 2′-OMe modified 2′-β-D-deoxyribosyl sugar moiety.
1562. The oligomeric compound of any of embodiments 1461-1544, wherein the safety enhancing nucleoside comprises a 3′-modified furanosyl sugar moiety having a 3′ substituent selected from: halo, allyl, amino, azido, SH, CN, CF₃, OCF₃, C₁-C₁₀ alkyl, C₁-C₁₀ substituted alkyl, S-alkyl, N(Rₘ)-alkyl, N(Rₘ)-alkenyl, N(Rₘ)-alkynyl, O-alkylenyl-O-alkyl, alkynyl, alkaryl, or aralkyl.
1563. The oligomeric compound of any of embodiments 1461-1544 or embodiment 1562, wherein the safety enhancing nucleoside comprises a 3′-modified furanosyl sugar moiety having a 3′ substituent selected from: C₁-C₁₀ alkyl, or C₁-C₁₀ substituted alkyl.
1564. The oligomeric compound of any of embodiments 1461-1544 or 1562-1563, wherein the safety enhancing nucleoside comprises a 3′-methyl furanosyl sugar moiety.
1565. The oligomeric compound of any of embodiments 1461-1544, wherein the safety enhancing nucleoside comprises a 4′-modified furanosyl sugar moiety having a 4′ substituent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
1566. The oligomeric compound of any of embodiments 1461-1544 or 1565, wherein the safety enhancing nucleoside comprises a 4′-modified furanosyl sugar moiety having a 4′-methyl.
1567. The oligomeric compound of any of embodiments 1461-1544, wherein the safety enhancing nucleoside has the structure shown below, wherein R represents an optional 2′ substituent group and Bx is a heterocyclic base moiety:

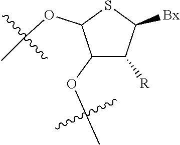

1568. The oligomeric compound of embodiment 1567, wherein in R is selected from among H, OH, OMe, F, or MOE.
1569. The oligomeric compound of any of embodiments 1461-1544, wherein the safety enhancing nucleoside comprises a 5′-modified furanosyl sugar moiety having a 5′ substiuent selected from: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl.
1570. The oligomeric compound of any of embodiments 1461-1544 or 1569, wherein the safety enhancing nucleoside comprises a 5′-modified furanosyl sugar moiety with a 5′ substituent selected from: 5′-methyl, 5′-ethyl or a 5′-allyl.
1571. The oligomeric compound of any of embodiments 1461-1544 or 1569-1570, wherein the safety enhancing nucleoside comprises a 5′-modified furanosyl sugar moiety with a 5′ substituent selected from: 5′-(R)-methyl- or 5′-(R)-ethyl.
1572. The oligomeric compound of any of embodiments 1461-1544 or 1569-1571, wherein the safety enhancing nucleoside comprises a 5′-(R)-methyl-2′-β-D-deoxyribosyl sugar moiety.
1573. The oligomeric compound of any of embodiments 1461-154 or 1569-1572, wherein the safety enhancing nucleoside comprises a 5′-(R)-ethyl-2′-β-D-deoxyribosyl sugar moiety.
1574. The oligomeric compound of any of embodiments 1461-1573, wherein the safety enhancing nucleoside does not comprise a 2′-F modified sugar moiety.
1575. The oligomeric compound of any of embodiments 1461-1574, wherein the safety enhancing nucleoside does not comprise a cEt modified sugar moiety.
1576. The oligomeric compound of any of embodiments 1461-1575, wherein the safety enhancing nucleoside does not comprise a 2′-MOE modified sugar moiety.
1577. The oligomeric compound of any of embodiments 1461-1576, wherein the safety enhancing nucleoside comprises a hypoxanthine nucleobase.
1578. The oligomeric compound of any of embodiments 1461-1577, wherein the safety enhancing nucleoside comprises a nucleobase selected from among thymine, uracil, guanine, cytosine, 5-methylcytosine, and adenine.
1579. The oligomeric compound of any of embodiments 1461-1578, wherein the safety enhancing nucleoside is a modified nucleoside other than cEt, MOE, LNA, or FANA.
1580. The oligomeric compound of any of embodiments 1461-1579, wherein each Nd is independently selected from among an unmodified 2′-β-D-deoxyribosyl sugar moiety, a DNA isomer, a 2′-modified DNA isomer, and a DNA mimic.
1581. The oligomeric compound of embodiment 1580, wherein each DNA isomer comprises a sugar moiety selected from among 2′-β-D-deoxyxylosyl, 2′-α-L-deoxyribosyl, 2′-β-L-deoxyribosyl, 2′-α-D-deoxyxylosyl, 2′-α-D-deoxyribosyl, 2′-α-L-deoxyxylosyl, and 2′-β-L-deoxyxylosyl.
1582. The oligomeric compound of embodiment 1581, wherein each DNA isomer comprises a sugar moiety selected from among 2′-β-D-deoxyxylosyl, 2′-α-L-deoxyribosyl, and 2′-β-L-deoxyribosyl.
1583. The oligomeric compound of embodiment 1580, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1,2′-α-L-deoxyxylosyl, and 2′-β-L-deoxyxylosyl and the 2′ position is further substituted with a hydroxyl, a fluoro, or O—C₁-C₁₀ alkoxy substituent.
1584. The oligomeric compound of embodiment 1583, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1,2′-α-L-deoxyxylosyl, and 2′-β-L-deoxyxylosyl and the 2′ position is further substituted with a hydroxyl, a fluoro, or OMe substituent.
1585. The oligomeric compound of embodiment 1584, wherein each modified DNA isomer comprises a 2′-modified sugar moiety, wherein the sugar moiety has the conformation 2′-β-D-deoxyxylosyl, 2′-α-L-deoxyribosyl, 2′-β-L-deoxyribosyl, 2′-α-D-deoxyxylosyl, 1,2′-α-L-deoxyxylosyl, and 2′-β-L-deoxyxylosyl and the 2′ position is further substituted with a fluoro or OMe substituent.
1586. The oligomeric compound of embodiment 1585, wherein each modified DNA isomer comprises a 2′-modified sugar moiety, wherein the sugar moiety is selected from among 2′-β-D-deoxyxylosyl, 2′-α-L-deoxyribosyl, and 2′-β-L-deoxyribosyl and the 2′ position is further substituted with a hydroxyl, fluoro, or OMe substituent.
1587. The oligomeric compound of embodiment 1580, wherein each DNA mimic comprises a structure represented by one of the formulas below:

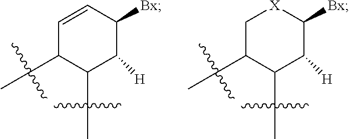

wherein X is O or S and Bx represents a heterocylic base moiety.
1588. The oligomeric compound of embodiment 1580, wherein each DNA mimic comprises a structure represented by one of the formulas below:

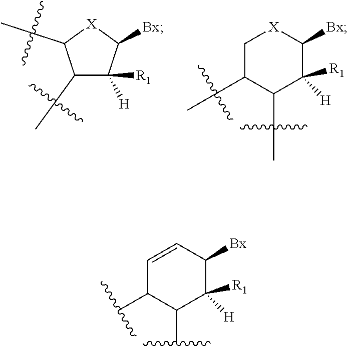

wherein X is O or S;
Bx represents a heterocyclic base moiety; and
R₁ is selected from among H, OH, halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH2C(═O)—N(Rₘ)(Rₙ), where each Rₘ and Rₙ is, independently, H, an amino protecting group, or substituted or unsubstituted C₁-C₁₀ alkyl;
wherein if the DNA mimic comprises the structure:

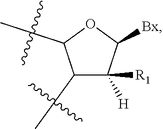

R₁ is other than H.
1589. The oligomeric compound of embodiment 1588, wherein R₁ is H, OH, OMe, or F.
1590. The oligomeric compound of embodiment 1588, wherein R₁ is not F.
1591. The oligomeric compound of embodiment 1580, wherein each DNA mimic comprises a structure represented by the formula below:

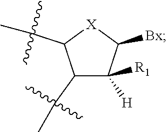

wherein X is S, Bx represents a heterocyclic base moiety, and R₁ is H.
1592. The oligomeric compound of embodiment 1580, wherein the DNA mimic has a sugar moiety selected from among: 5′-methyl-2′-β-D-deoxyribosyl, 5′-ethyl-2′-β-D-deoxyribosyl, and 5′-allyl-2′-β-D-deoxyribosyl.
1593. The oligomeric compound of embodiment 1580, wherein the DNA mimic comprises a 2′-fluoro-β-D-arabinofuranosyl sugar moiety.
1594. The oligomeric compound of embodiment 1580, wherein the DNA mimic does not comprise a 2′-fluoro-β-D-arabinofuranosyl sugar moiety.
1595. The oligomeric compound of any of embodiments 1461-1594, wherein each Na is an unmodified 2′-β-D-deoxyribosyl sugar moiety.
1596. The oligomeric compound of any of embodiments 1461-1595, wherein the central region comprises no more than four nucleosides selected from among DNA isomers, modified DNA isomers, and DNA mimics.
1597. The oligomeric compound of any of embodiments 1461-1595, wherein the central region comprises no more than three nucleosides selected from among DNA isomers, modified DNA isomers, and DNA mimics.
1598. The oligomeric compound of any of embodiments 1461-1595, wherein the central region comprises no more than two nucleosides selected from among DNA isomers, modified DNA isomers, and DNA mimics.
1599. The oligomeric compound of any of embodiments 1461-1595, wherein the central region comprises no more than one nucleoside selected from among DNA isomers, modified DNA isomers, and DNA mimics.
1600. The oligomeric compound of any of embodiments 1461-1599, wherein the central region contains exactly one safety enhancing nucleoside and the remainder of nucleosides in the central region are unmodified 2′-β-D-deoxyribosyl sugar moieties.
1601. The oligomeric compound of any of embodiments 1461-1600, wherein at least one internucleoside linkage of the central region selected from among: the internucleoside Nda to Nx, Nx to Ny, Ny to Nz, or Nz to the adjacent Ndb is a neutral internucleoside linkage.
1602. The oligomeric compound of embodiment 1601, wherein the neutral linkage is a phosphonate internucleoside linkage.
1603. The oligomeric compound of embodiment 1601, wherein the neutral linkage is a methoxypropyl internucleoside linkage.
1604. The oligomeric compound of embodiment 1601, wherein each remaining internucleoside linkage is a phosphodiester or phosphorothioate internucleoside linkage.
1605. The oligomeric compound of any of embodiments 1461-1600, wherein at least one internucleoside linkage of the central region is a 2′-5′ internucleoside linkage.
1606. The oligomeric compound of any of embodiments 1461-1600, wherein exactly one internucleoside linkage of the central region is a 2′-5′ internucleoside linkage.
1607. The oligomeric compound of any of embodiments 1461-1600, wherein at least one internucleoside linkage of the central region selected from among: the internucleoside Nda to Nx, Nx to Ny, Ny to Nz, or Nz to the adjacent Ndb is a a 2′-5′ internucleoside linkage.
1608. A chirally enriched population of modified oligonucleotides of any of embodiments 1461-1607, wherein the central region has at least one particular phorphorothioate internucleoside linkage having a particular stereochemical configuration.
1609. The chirally enriched population of embodiment 1608, wherein the central region has at least one phorphorothioate internucleoside linkage having the (Sp) configuration.
1610. The chirally enriched population of embodiment 1608, wherein central region has at least one phorphorothioate internucleoside linkage having the (Rp) configuration.
1611. The chirally enriched population of embodiment 1608, wherein the central region has a particular, independently selected stereochemical configuration at each phosphorothioate internucleoside linkage.
1612. The chirally enriched population of embodiment 1608, wherein the each phosphorothioate internucleoside linkage of the central region has the (Sp) configuration.
1613. The chirally enriched population of embodiment 1608, wherein the central region has one phosphorothioate internucleoside linkage having the (Rp) configuration and wherein each remaining central region phosphorothioate internucleoside linkage has the (Sp) configuration.
1614. The chirally enriched population of embodiment 1608, wherein the central region has an (Rp) phosphorothioate internucleoside linkage adjacent to an (Sp) phosphorothioate internucleoside linkage.
1615. The chirally enriched population of embodiment 1608, wherein the central region has an (Rp) phosphorothioate internucleoside linkage adjacent to a second (Rp) phosphorothioate internucleoside linkage.
1616. The chirally enriched population of embodiment 1608, wherein the central region has an (Rp) phosphorothioate internucleoside linkage adjacent to a second (Rp) phosphorothioate internucleoside linkage, and wherein each remaining central region phosphorothioate internucleoside linkage has the (Sp) configuration.
1617. The chirally enriched population of any of embodiments 1609, 1610, 1614, or 1615 wherein each phosphorothioate internucleoside linkage that does not have the (Rp) or (Sp) configuration is stereorandom.
1618. The oligomeric compound of any of embodiments 1461-1617 comprising a conjugate group.
1619. The oligomeric compound of embodiment 1618, wherein the conjugate group comprises a linking group attaching the remainder of the conjugate group to the modified oligonucleotide, wherein the linking group comprises 1-5 nucleosides.
1620. The oligomeric compound of any of embodiments 1461-1618, wherein the oligomeric compound does not comprise additional nucleosides beyond those of the modified oligonucleotide.
1621. The oligomeric compound of any of embodiments 1461-1620, wherein the nucleobase sequence of the modified oligonucleotide is complementary to a target RNA.
1622. The oligomeric compound of embodiment 1621, wherein the nucleobase sequence of the modified oligonucleotide is at least 85% complementary to the target RNA.
1623. The oligomeric compound of embodiment 1621, wherein the nucleobase sequence of the modified oligonucleotide is at least 90% complementary to the target RNA.
1624. The oligomeric compound of embodiment 1621, wherein the nucleobase sequence of the modified oligonucleotide is at least 95% complementary to the target RNA.
1625. The oligomeric compound of embodiment 1621, wherein the nucleobase sequence of the modified oligonucleotide is 100% complementary to the target RNA.
1626. The oligomeric compound of any of embodiments 1621-1625, wherein the target RNA is a target mRNA or a target pre-mRNA.
1627. The oligomeric compound of embodiment 1626, wherein the target mRNA or target pre-mRNA is not a mouse, rat, monkey, or human PTEN RNA.
1628. The oligomeric compound of embodiment 1626 or 1627, wherein the target mRNA or target pre-mRNA is not a mouse, rat, monkey, or human SRB-1 RNA.
1629. The oligomeric compound of any of embodiments 1626-1628, wherein the target mRNA or target pre-mRNA is not a mouse, rat, monkey, or human MYD88 RNA.
1630. The oligomeric compound of any of embodiments 1626-1629, wherein the target mRNA or target pre-mRNA is not a mouse, rat, monkey, or human HTT RNA, is not a mouse, rat, monkey, or human SOD1 RNA, and is not a mouse, rat, monkey, or human alpha-synuclein RNA.
1631. The oligomeric compound of any of embodiments 1621-1630, wherein the target RNA is a human RNA.
1632. The oligomeric compound of any of embodiments 1621-1631, wherein the target RNA is expressed in the liver.
1633. The oligomeric compound of any of embodiments 1621-1632, wherein the target RNA is a liver target.
1634. The oligomeric compound of any of embodiments 1621-1631, wherein the target RNA is not expressed in the liver.
1635. The oligomeric compound of any of embodiments 1621-1631 or 1634, wherein the target RNA is not a liver target.
1636. The oligomeric compound of any of embodiments 1621-1635, wherein modulation of the expression of the target RNA in the liver is associated with treating a disorder or condition.
1637. The oligomeric compound of embodiment 1636, wherein the disorder or condition is a liver disorder or condition.
1638. The oligomeric compound of any of embodiments 1621-1637, wherein the target RNA is expressed in the central nervous system.
1639. The oligomeric compound of any of embodiments 1621-1637, wherein the target RNA is not expressed in the central nervous system.
1640. The oligomeric compound of any of embodiments 1621-1638, wherein the target RNA is a central nervous system target.
1641. The oligomeric compound of any of embodiments 1621-1639, wherein the target RNA is not a central nervous system target.
1642. The oligomeric compound of any of embodiments 1621-1641, wherein the target RNA is expressed in white fat cells.
1643. The oligomeric compound of any of embodiments 1621-1641, wherein the target RNA is expressed in brown fat cells.
1644. The oligomeric compound of any of embodiments 1621-1641, wherein the target RNA is expressed in adipocytes.
1645. The oligomeric compound of any of embodiments 1621-1641, wherein the target RNA is expressed in macrophages.
1646. The oligomeric compound of any of embodiments 1621-1641, wherein the target RNA is expressed in cancer cells.
1647. The oligomeric compound of any of embodiments 1621-1641, wherein the target RNA is expressed in tumor cells.
1648. The oligomeric compound of any of embodiments 1621-1641, wherein the target RNA is expressed in smooth muscle cells.
1649. The oligomeric compound of any of embodiments 1621-1641, wherein the target RNA is expressed in lymphocytes.
1650. The oligomeric compound of any of embodiments 1621-1641, wherein the target RNA is expressed in pulmonary cells.
1651. The oligomeric compound of any of embodiments 1621-1641, wherein the target RNA is expressed in heart muscle cells.
1652. The oligomeric compound of any of embodiments 1621-1641, wherein the target RNA is expressed in cardiomyocytes.
1653. The oligomeric compound of any of embodiments 1621-1641, wherein the target RNA is expressed in endothelial cells.
1654. The oligomeric compound of any of embodiments 1621-1641, wherein the target RNA is expressed in fibroblasts.
1655. The oligomeric compound of any of embodiments 1621-1641, wherein the target RNA is expressed in glial cells.
1656. The oligomeric compound of any of embodiments 1621-1641, wherein the target RNA is expressed in Schwann cells.
1657. The oligomeric compound of any of embodiments 1621-1641, wherein the target RNA is expressed in pancreatic cells.
1658. The oligomeric compound of any of embodiments 1621-1641, wherein the target RNA is expressed in kidney cells.
1659. The oligomeric compound of any of embodiments 1621-1641, wherein the target RNA is expressed in beta cells.
1660. The oligomeric compound of any of embodiments 1621-1641, wherein the target RNA is expressed in non-parenchymal cells.
1661. The oligomeric compound of any of embodiments 1621-1641, wherein the target RNA is expressed in hepatocytes.
1662. The oligomeric compound of any of embodiments 1621-1641, wherein the target RNA is expressed in oligodendrocytes.
1663. The oligomeric compound of any of embodiments 1621-1641, wherein the target RNA is expressed in astrocytes.
1664. The oligomeric compound of any of embodiments 1621-1641, wherein the target RNA is expressed in microglia.
1665. The oligomeric compound of any of embodiments 1621-1641, wherein the target RNA is expressed in ependymal cells.
1666. The oligomeric compound of any of embodiments 1621-1641, wherein the target RNA is expressed in sensory neurons.
1667. The oligomeric compound of any of embodiments 1621-1641, wherein the target RNA is expressed in motor neurons.
1668. The oligomeric compound of any of embodiments 1621-1641, wherein the target RNA is expressed in skeletal muscle.
1669. The oligomeric compound of any of embodiments 1621-1641, wherein the target RNA is expressed in cardiac muscle.
1670. The oligomeric compound of any of embodiments 1621-1641, wherein the target RNA is expressed in smooth muscle.
1671. The oligomeric compound of any of embodiments 1621-1641, wherein the target RNA is expressed in adipose tissue.
1672. The oligomeric compound of any of embodiments 1621-1641, wherein the target RNA is expressed in white adipose tissue.
1673. The oligomeric compound of any of embodiments 1621-1641, wherein the target RNA is expressed in the spleen.
1674. The oligomeric compound of any of embodiments 1621-1641, wherein the target RNA is expressed in the bone.
1675. The oligomeric compound of any of embodiments 1621-1641, wherein the target RNA is expressed in the bone marrow.
1676. The oligomeric compound of any of embodiments 1621-1641, wherein the target RNA is expressed in the intestine.
1677. The oligomeric compound of any of embodiments 1621-1641, wherein the target RNA is expressed in the adrenal glands.
1678. The oligomeric compound of any of embodiments 1621-1641, wherein the target RNA is expressed in the testes.
1679. The oligomeric compound of any of embodiments 1621-1641, wherein the target RNA is expressed in the ovaries.
1680. The oligomeric compound of any of embodiments 1621-1641, wherein the target RNA is expressed in the pancreas.
1681. The oligomeric compound of any of embodiments 1621-1641, wherein the target RNA is expressed in the pituitary gland.
1682. The oligomeric compound of any of embodiments 1621-1641, wherein the target RNA is expressed in the prostate gland.
1683. The oligomeric compound of any of embodiments 1621-1641, wherein the target RNA is expressed in the skin.
1684. The oligomeric compound of any of embodiments 1621-1641, wherein the target RNA is expressed in the epidermis.
1685. The oligomeric compound of any of embodiments 1621-1641, wherein the target RNA is expressed in the uterus.
1686. The oligomeric compound of any of embodiments 1621-1641, wherein the target RNA is expressed in the bladder.
1687. The oligomeric compound of any of embodiments 1621-1641, wherein the target RNA is expressed in the brain.
1688. The oligomeric compound of any of embodiments 1621-1641, wherein the target RNA is expressed in the glomerulus.
1689. The oligomeric compound of any of embodiments 1621-1641, wherein the target RNA is expressed in the distal tubular epithelium.
1690. The oligomeric compound of any of embodiments 1621-1641, wherein the target RNA is expressed in breast tissue.
1691. The oligomeric compound of any of embodiments 1621-1641, wherein the target RNA is expressed in the lung.
1692. The oligomeric compound of any of embodiments 1621-1641, wherein the target RNA is expressed in the heart.
1693. The oligomeric compound of any of embodiments 1621-1641, wherein the target RNA is expressed in the kidney.
1694. The oligomeric compound of any of embodiments 1621-1641, wherein the target RNA is expressed in the ganglion.
1695. The oligomeric compound of any of embodiments 1621-1641, wherein the target RNA is expressed in the frontal cortex.
1696. The oligomeric compound of any of embodiments 1621-1641, wherein the target RNA is expressed in the spinal cord.
1697. The oligomeric compound of any of embodiments 1621-1641, wherein the target RNA is expressed in the trigeminal ganglion.
1698. The oligomeric compound of any of embodiments 1621-1641, wherein the target RNA is expressed in the sciatic nerve.
1699. The oligomeric compound of any of embodiments 1621-1641, wherein the target RNA is expressed in the dorsal root ganglion.
1700. The oligomeric compound of any of embodiments 1621-1641, wherein the target RNA is expressed in the epidymal fat.
1701. The oligomeric compound of any of embodiments 1621-1641, wherein the target RNA is expressed in the diaphragm.
1702. The oligomeric compound of any of embodiments 1621-1641, wherein the target RNA is expressed in the colon.
1703. The oligomeric compound of any of embodiments 1621-1641, wherein the target RNA is a white fat cell target.
1704. The oligomeric compound of any of embodiments 1621-1641, wherein the target RNA is a brown fat cell target.
1705. The oligomeric compound of any of embodiments 1621-1641, wherein the target RNA is an adipocyte target.
1706. The oligomeric compound of any of embodiments 1621-1641, wherein the target RNA is a macrophage target.
1707. The oligomeric compound of any of embodiments 1621-1641, wherein the target RNA is a cancer cell target.
1708. The oligomeric compound of any of embodiments 1621-1641, wherein the target RNA is a tumor cell target.
1709. The oligomeric compound of any of embodiments 1621-1641, wherein the target RNA is a smooth muscle cell target.
1710. The oligomeric compound of any of embodiments 1621-1641, wherein the target RNA is a lymphocyte target.
1711. The oligomeric compound of any of embodiments 1621-1641, wherein the target RNA is a pulmonary cell target.
1712. The oligomeric compound of any of embodiments 1621-1641, wherein the target RNA is a heart muscle cell target.
1713. The oligomeric compound of any of embodiments 1621-1641, wherein the target RNA is a cardiomyocyte target.
1714. The oligomeric compound of any of embodiments 1621-1641, wherein the target RNA is a endothelial cell target.
1715. The oligomeric compound of any of embodiments 1621-1641, wherein the target RNA is a fibroblast target.
1716. The oligomeric compound of any of embodiments 1621-1641, wherein the target RNA is a glial cell target.
1717. The oligomeric compound of any of embodiments 1621-1641, wherein the target RNA is a Schwann cell target.
1718. The oligomeric compound of any of embodiments 1621-1641, wherein the target RNA is a pancreatic cell target.
1719. The oligomeric compound of any of embodiments 1621-1641, wherein the target RNA is a kidney cell target.
1720. The oligomeric compound of any of embodiments 1621-1641, wherein the target RNA is a beta cell target.
1721. The oligomeric compound of any of embodiments 1621-1641, wherein the target RNA is a non-parenchymal cell target.
1722. The oligomeric compound of any of embodiments 1621-1641, wherein the target RNA is a hepatocyte target.
1723. The oligomeric compound of any of embodiments 1621-1641, wherein the target RNA a oligodendrocyte target.
1724. The oligomeric compound of any of embodiments 1621-1641, wherein the target RNA is a astrocyte target.
1725. The oligomeric compound of any of embodiments 1621-1641, wherein the target RNA is a microglia target.
1726. The oligomeric compound of any of embodiments 1621-1641, wherein the target RNA is a ependymal cell target.
1727. The oligomeric compound of any of embodiments 1621-1641, wherein the target RNA is a sensory neuron target.
1728. The oligomeric compound of any of embodiments 1621-1641, wherein the target RNA is a motor neuron target.
1729. The oligomeric compound of any of embodiments 1621-1641, wherein the target RNA is a skeletal muscle target.
1730. The oligomeric compound of any of embodiments 1621-1641, wherein the target RNA is a cardiac muscle target.
1731. The oligomeric compound of any of embodiments 1621-1641, wherein the target RNA is a smooth muscle target.
1732. The oligomeric compound of any of embodiments 1621-1641, wherein the target RNA is a adipose tissue target.
1733. The oligomeric compound of any of embodiments 1621-1641, wherein the target RNA is a white adipose tissue target.
1734. The oligomeric compound of any of embodiments 1621-1641, wherein the target RNA is a spleen target.
1735. The oligomeric compound of any of embodiments 1621-1641, wherein the target RNA is a bone target.
1736. The oligomeric compound of any of embodiments 1621-1641, wherein the target RNA is a bone marrow target.
1737. The oligomeric compound of any of embodiments 1621-1641, wherein the target RNA is an intestinal target.
1738. The oligomeric compound of any of embodiments 1621-1641, wherein the target RNA is an adrenal gland target.
1739. The oligomeric compound of any of embodiments 1621-1641, wherein the target RNA is a testicular target.
1740. The oligomeric compound of any of embodiments 1621-1641, wherein the target RNA is an ovarian target.
1741. The oligomeric compound of any of embodiments 1621-1641, wherein the target RNA is a pancreatic target.
1742. The oligomeric compound of any of embodiments 1621-1641, wherein the target RNA is a pituitary gland target.
1743. The oligomeric compound of any of embodiments 1621-1641, wherein the target RNA is a prostate gland target.
1744. The oligomeric compound of any of embodiments 1621-1641, wherein the target RNA is a skin target.
1745. The oligomeric compound of any of embodiments 1621-1641, wherein the target RNA is an epidermal target.
1746. The oligomeric compound of any of embodiments 1621-1641, wherein the target RNA is a uterine target.
1747. The oligomeric compound of any of embodiments 1621-1641, wherein the target RNA is a bladder target.
1748. The oligomeric compound of any of embodiments 1621-1641, wherein the target RNA is a brain target.
1749. The oligomeric compound of any of embodiments 1621-1641, wherein the target RNA is a glomerulus target.
1750. The oligomeric compound of any of embodiments 1621-1641, wherein the target RNA is a distal tubular epithelium target.
1751. The oligomeric compound of any of embodiments 1621-1641, wherein the target RNA is abreast tissue target.
1752. The oligomeric compound of any of embodiments 1621-1641, wherein the target RNA is a lung target.
1753. The oligomeric compound of any of embodiments 1621-1641, wherein the target RNA is a heart target.
1754. The oligomeric compound of any of embodiments 1621-1641, wherein the target RNA is a kidney target.
1755. The oligomeric compound of any of embodiments 1621-1641, wherein the target RNA is a ganglion target.
1756. The oligomeric compound of any of embodiments 1621-1641, wherein the target RNA is a frontal cortex target.
1757. The oligomeric compound of any of embodiments 1621-1641, wherein the target RNA is a spinal cord target.
1758. The oligomeric compound of any of embodiments 1621-1641, wherein the target RNA is a trigeminal ganglion target.
1759. The oligomeric compound of any of embodiments 1621-1641, wherein the target RNA is a sciatic nerve target.
1760. The oligomeric compound of any of embodiments 1621-1641, wherein the target RNA is a dorsal root ganglion target.
1761. The oligomeric compound of any of embodiments 1621-1641, wherein the target RNA is a epidymal fat target.
1762. The oligomeric compound of any of embodiments 1621-1641, wherein the target RNA is a diaphragm target.
1763. The oligomeric compound of any of embodiments 1621-1641, wherein the target RNA is a colon target.
1764. The oligomeric compound of any of embodiments 1621-1763, wherein the target RNA is a MeCP2, DUX4, HDAC2, APP, Ataxin 1, Ataxin 2, Ataxin 3, Ataxin 6, Ataxin 7, C9ORF72, UBE3A, Prion, PMP22, Tau, LRRK2, LINGO2, GYS1, KCNT1, IRF8, Progranulin, or GFAP RNA.
1765. The oligomeric compound of any of embodiments 1621-1763, wherein the target RNA is a HTT RNA.
1766. The oligomeric compound of embodiment 1764, wherein the target RNA is a MeCP2 RNA.
1767. The oligomeric compound of embodiment 1764, wherein the target RNA is a DUX4 RNA.
1768. The oligomeric compound of embodiment 1764, wherein the target RNA is a HDAC2 RNA.
1769. The oligomeric compound of embodiment 1764, wherein the target RNA is a Ataxin 1 RNA.
1770. The oligomeric compound of embodiment 1764, wherein the target RNA is a Ataxin 2 RNA.
1771. The oligomeric compound of embodiment 1764, wherein the target RNA is a Ataxin 3 RNA.
1772. The oligomeric compound of embodiment 1764, wherein the target RNA is a Ataxin 6 RNA.
1773. The oligomeric compound of embodiment 1764, wherein the target RNA is a Ataxin 7 RNA.
1774. The oligomeric compound of embodiment 1764, wherein the target RNA is a C9ORF72 RNA.
1775. The oligomeric compound of embodiment 1621-1763, wherein the target RNA is an alpha-synuclein RNA.
1776. The oligomeric compound of embodiment 1764, wherein the target RNA is an UBE3A RNA.
1777. The oligomeric compound of any of embodiments 1621-1763, wherein the target RNA is a SOD1 RNA.
1778. The oligomeric compound of embodiment 1764, wherein the target RNA is a Prion RNA.
1779. The oligomeric compound of embodiment 1764, wherein the target RNA is a PMP22 RNA.
1780. The oligomeric compound of any of embodiments 1621-1764, wherein the target RNA is a Tau RNA.
1781. The oligomeric compound of embodiment 1764, wherein the target RNA is a LRRK2 RNA.
1782. The oligomeric compound of embodiment 1764, wherein the target RNA is an APP RNA.
1783. The oligomeric compound of 1764, wherein the target RNA is a LING02 RNA.
1784. The oligomeric compound of any of embodiments 1764, wherein the target RNA is a GYS1 RNA.
1785. The oligomeric compound of any of embodiments 1764, wherein the target RNA is a KCNT1 RNA.
1786. The oligomeric compound of any of embodiments 1764, wherein the target RNA is a IRF8 RNA.
1787. The oligomeric compound of any of embodiments 1764, wherein the target RNA is a Progranulin RNA.
1788. The oligomeric compound of any of embodiments 1764, wherein the target RNA is a GFAP RNA.
1789. The oligomeric compound of any of embodiments 1621-1788, wherein modulation of the expression of the target RNA is associated with treating a disorder or condition.
1790. The oligomeric compound of embodiment 1789, wherein the disorder or condition is Alzheimer's Disease, Amyotrophic Lateral Sclerosis, Parkinson's Disease, a Spinocerebellar ataxia, Angelman Syndrome, Alexander's Disease, Lafora Disease, Charcot-Marie Tooth Disease, Prion Disease, a dementia, neurodegeneration, MeCP2 Duplication Syndrome, encephalopathy, neuroinflammation, or multiple sclerosis.
1791. The oligomeric compound of embodiment 1790, wherein the disorder or condition is Alzheimer's Disease.
1792. The oligomeric compound of embodiment 1790, wherein the disorder or condition is Amyotrophic Lateral Sclerosis.
1793. The oligomeric compound of embodiment 1790, wherein the disorder or condition is Parkinson's Disease.
1794. The oligomeric compound of embodiment 1790 wherein the disorder or condition is a Spinocerebellar ataxia.
1795. The oligomeric compound of embodiment 1790 wherein the disorder or condition is Angelman Syndrome.
1796. The oligomeric compound of embodiment 1790, wherein the disorder or condition is Alexander's Disease.
1797. The oligomeric compound of embodiment 1790, wherein the disorder or condition is Lafora Disease.
1798. The oligomeric compound of embodiment 1790, wherein the disorder or condition is Charcot-Marie Tooth Disease.
1799. The oligomeric compound of embodiment 1790, wherein the disorder or condition is Prion Disease.
1800. The oligomeric compound of embodiment 1790, wherein the disorder or condition is a dementia.
1801. The oligomeric compound of embodiment 1790, wherein the disorder or condition is neurodegeneration.
1802. The oligomeric compound of embodiment 1790, wherein the disorder or condition is MeCP2 Duplication Syndrome.
1803. The oligomeric compound of embodiment 1790, wherein the disorder or condition is encephalopathy.
1804. The oligomeric compound of embodiment 1790, wherein the disorder or condition is neuroinflammation.
1805. The oligomeric compound of embodiment 1790, wherein the disorder or condition is multiple sclerosis.
1806. The oligomeric compound of any of embodiments 1461-1805, wherein the parent oligomeric compound of the oligomeric compound of any of embodiments 1461-1805 is cytotoxic in vitro.
1807. The oligomeric compound of embodiment 1806, wherein the parent oligomeric compound is cytotoxic in a standard in vitro cytotoxicity assay.
1808. The oligomeric compound of any of embodiments 1461-1805 wherein administration to a mouse of the parent oligomeric compound of the oligomeric compound of any of embodiments 1461-1805 is hepatotoxic to the mouse.
1809. The oligomeric compound of embodiment 1808, wherein the administration of the parent oligomeric compound to the mouse is systemic administration.
1810. The oligomeric compound of embodiment 1809, wherein the systemic administration is subcutaneous administration.
1811. The oligomeric compound of any of embodiments 1808-1810, wherein the mouse is a CD-1 mouse.
1812. The oligomeric compound of any of embodiments 1808-1810, wherein the mouse is a C57BL/6 mouse.
1813. The oligomeric compound of any of embodiments 1808-1810, wherein the mouse is a BALB/c mouse.
1814. The oligomeric compound of any of embodiments 1807-1813, wherein the plasma ALT level in the mouse is higher than 500 units per liter following the administration of the parent oligomeric compound.
1815. The oligomeric compound of any of embodiments 1807-1814, wherein the plasma AST level in the mouse is higher than 500 units per liter following the administration of the parent oligomeric compound.
1816. The oligomeric compound of any of embodiments 1807-1815, wherein the plasma ALT level in the mouse is higher than 1000 units per liter following the administration of the parent oligomeric compound.
1817. The oligomeric compound of any of embodiments 1807-1816, wherein the plasma AST level in the mouse is higher than 1000 units per liter following the administration of the parent oligomeric compound.
1818. The oligomeric compound of any of embodiments 1807-1817, wherein the hepatotoxicity is measured 24-48 hours following the administration of the parent oligomeric compound.
1819. The oligomeric compound of any of embodiments 1807-1817, wherein the hepatotoxicity is measured 72 hours following the administration of the parent oligomeric compound.
1820. The oligomeric compound of any of embodiments 1807-1817, wherein the hepatotoxicity is measured 3 days following the administration of the parent oligomeric compound.
1821. The oligomeric compound of any of embodiments 1807-1817, wherein the hepatotoxicity is measured 5 days following the administration of the parent oligomeric compound.
1822. The oligomeric compound of any of embodiments 1807-1817, wherein the hepatotoxicity is measured 7 days following the administration of the parent oligomeric compound.
1823. The oligomeric compound of any of embodiments 1807-1817, wherein the hepatotoxicity is measured 10 days following the administration of the parent oligomeric compound.
1824. The oligomeric compound of any of embodiments 1807-1817, wherein the hepatotoxicity is measured 14 days following the administration of the parent oligomeric compound.
1825. The oligomeric compound of any of embodiments 1807-1817, wherein 1-10 mg/kg of the parent oligomeric compound is administered to the mouse.
1826. The oligomeric compound of any of embodiments 1807-1817, wherein 10-25 mg/kg of the parent oligomeric compound is administered to the mouse.
1827. The oligomeric compound of any of embodiments 1807-1817, wherein 25-50 mg/kg of the parent oligomeric compound is administered to the mouse.
1828. The oligomeric compound of any of embodiments 1807-1817, wherein 50-100 mg/kg of the parent oligomeric compound is administered to the mouse.
1829. The oligomeric compound of any of embodiments 1807-1817, wherein 100-150 mg/kg of the parent oligomeric compound is administered to the mouse.
1830. The oligomeric compound of any of embodiments 1807-1817, wherein the mouse is a BALB/c mouse, wherein 150 mg/kg of the parent oligomeric compound is administered to the mouse, and wherein the plasma ALT level in the mouse is measured 72 hours following the administration of the parent oligomeric compound.
1831. The oligomeric compound of any of embodiments 1461-1830, wherein administration of 50 mg/kg of the oligomeric compound of any of embodiments 1461-1830 to a mouse is not hepatotoxic to the mouse.
1832. The oligomeric compound of embodiment 1831, wherein plasma ALT level in the mouse is lower than 100 units per liter following the administration of the oligomeric compound of embodiment 1831.
1833. The oligomeric compound of embodiment 1831 or 1832, wherein plasma ALT level in the mouse is at least 10-fold lower than plasma ALT level in a mouse administered the parent oligomeric compound of the oligomeric compound of embodiment 1831 or 1832, wherein the administrations and plasma ALT measurements of both the oligomeric compound of embodiment 1831 or 1832 and the parent oligomeric compound are completed in the same way.
1834. The oligomeric compound of embodiment 1833, wherein the plasma ALT level in the mouse is at least 100-fold lower than the plasma ALT level in the mouse administered the parent oligomeric compound.
1835. The oligomeric compound of embodiment 1833, wherein the plasma ALT level in the mouse is at least 500-fold lower than the plasma ALT level in the mouse administered the parent oligomeric compound.
1836. The oligomeric compound of any of embodiments 1807-1835, wherein the therapeutic index in a mouse of the oligomeric compound of any of embodiments 1807-1835 is increased relative to the therapeutic index of the parent oligomeric compound.
1837. The oligomeric compound of embodiment 1836, wherein the therapeutic index in a mouse of the oligomeric compound of embodiment 1836 is at least two-fold greater than the therapeutic index of the parent oligomeric compound.
1838. The oligomeric compound of any of embodiments 1461-1805, wherein an otherwise identical oligomeric compound lacking the at least one modified internucleoside linkage other than phosphorothioate and/or at least one modified nucleoside comprising a modified nucleobase other than 5-methylcytosine and/or a modified sugar moiety in the central region is hepatotoxic following administration to a mouse;
   and wherein the otherwise identical oligomeric compound has a central region consisting of phosphorothioate internucleoside linkages, unmodified 2′-β-D-deoxyribosyl sugar moieties, and nucleobases selected from thymine, uracil, adenine, guanine, cytosine, and 5-methylcytosine.
1839. The oligomeric compound of embodiment 1838, wherein the at least one modified internucleoside linkage other than phosphorothioate and/or at least one modified nucleoside comprising a modified nucleobase other than 5-methylcytosine and/or a modified sugar moiety in the central region is a 2′-O-methyl modified sugar moiety.
1840. The oligomeric compound of embodiment 1839, wherein the 2′-O-methyl modified sugar moiety is at position 2 of the central region.
1841. The oligomeric compound of embodiment 1838-1840, wherein the otherwise identical oligomeric compound lacks a 2′-O-methyl modified sugar moiety, and wherein the otherwise identical oligomeric compound comprises an unmodified 2′-β-D-deoxyribosyl sugar moiety in place of the 2′-O-methyl modified sugar moiety.
1842. The oligomeric compound of any of embodiments 1838-1841, wherein the nucleobase sequence of the otherwise identical oligomeric compound is 100% complementary to the target RNA.
1843. The oligomeric compound of any of embodiments 1838-1842, wherein the otherwise identical oligomeric compound is a parent oligomeric compound of the oligomeric compound of any of embodiments 1838-1842.
1844. The oligomeric compound of any of embodiments 1621-1843, wherein the oligomeric compound inhibits the target RNA with an IC₅₀ lower than 100 nM in a standard in vitro activity assay.
1845. The oligomeric compound of any of embodiments 1621-1843, wherein the parent oligomeric compound of the oligomeric compound of any of embodiments 1621-1843 inhibits the target RNA with an IC₅₀ lower than 100 nM in a standard in vitro activity assay.
1846. The oligomeric compound of any of embodiments 1621-1845, wherein the difference between the IC₅₀ of the parent oligomeric compound measured in a standard in vitro activity assay and the IC₅₀ of the oligomeric compound of any of embodiments 1621-1845 measured in a standard in vitro activity assay is less than 4-fold.
1847. The oligomeric compound of any of embodiments 1621-1845, wherein the difference between the IC₅₀ of the parent oligomeric compound measured in a standard in vitro activity assay and the IC₅₀ of the oligomeric compound of any of embodiments 1621-1845 measured in a standard in vitro activity assay is less than 3-fold.
1848. The oligomeric compound of any of embodiments 1621-1845, wherein the difference between the IC₅₀ of the parent oligomeric compound measured in a standard in vitro activity assay and the IC₅₀ of the oligomeric compound of any of embodiments 1621-1845 measured in a standard in vitro activity assay is less than 2-fold.
1849. The oligomeric compound of any of embodiments 1621-1848, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
1850. The oligomeric compound of any of embodiments 1621-1849, wherein the nucleobase sequence of the parent oligomeric compound is 100% complementary to the target RNA.
1851. The oligomeric compound of any of embodiments 1461-1850, wherein administration of 10-50 mg of the oligomeric compound to a human is not hepatotoxic to the human.
1852. The oligomeric compound of any of embodiments 1461-1850, wherein administration of 50-100 mg of the oligomeric compound to a human is not hepatotoxic to the human.
1853. The oligomeric compound of any of embodiments 1461-1850, wherein administration of 100-300 mg of the oligomeric compound to a human is not hepatotoxic to the human.
1854. The oligomeric compound of any of embodiments 1851-1853, wherein the administration is systemic administration.
1855. A composition comprising the oligomeric compound of any of embodiments 1461-1854, and a second oligomeric compound, wherein the second oligomeric compound is complementary to the oligomeric compound of any of embodiments 1461-1854.
1856. The composition of embodiment 1855, wherein the second oligomeric compound comprises at least 14 contiguous linked nucleosides that are at least 90% complementary to the oligomeric compound of any of embodiments 1851-1853.
1857. The composition of embodiment 1855, wherein the second oligomeric compound comprises at least 14 contiguous linked nucleosides that are 100% complementary to the oligomeric compound of any of embodiments 1461-1854.
1858. A pharmaceutical composition comprising the oligomeric compound of any of embodiments 1461-1854 or the composition of any of embodiments 1855-1857, comprising a pharmaceutically acceptable carrier or diluent.
1859. A method comprising administering the oligomeric compound or composition of any of embodiments 1461-1858 to a human subject.
1860. A method of treating a disease or disorder comprising administering the oligomeric compound or composition of any of embodiments 1461-1858 to a human subject.
1861. A method of modulating the amount or activity of a target RNA in a human subject comprising administering the oligomeric compound or composition of any of embodiments 1461-1858 to the human subject; and thereby modulating the amount or activity of the target RNA in the human subject.
1862. A method of treating a disease or disorder in a human subject comprising administering the oligomeric compound or composition of any of embodiments 1461-1858 to the human subject having the disease or disorder; and thereby treating the disease or disorder in the human subject.
1863. The method of embodiment 1861 or 1862, wherein the disease or disorder is a liver disease, and wherein the oligomeric compound modulates the amount or activity of a liver target RNA.
1864. The method of embodiment 1861 or 1862, wherein the disease or disorder is a central nervous system disease, and wherein the oligomeric compound modulates the amount or activity of a central nervous system target RNA.
1865. The method of any of embodiments 1861-1864, wherein the disease or disorder is a cancer, a neurodegenerative disease, a cardiovascular disease, an immunological disease, a hereditary disease, a repeat expansion disease, a muscle-wasting disease, or a metabolic disease.
1866. The method of embodiment 1861 or 1862, wherein the oligomeric compound modulates the amount or activity of a white fat cell target.
1867. The method of embodiment 1861 or 1862, wherein the oligomeric compound modulates the amount or activity of a brown fat cell target.
1868. The method of embodiment 1861 or 1862, wherein the oligomeric compound modulates the amount or activity of an adipocyte target.
1869. The method of embodiment 1861 or 1862, wherein the oligomeric compound modulates the amount or activity of a macrophage target.
1870. The method of embodiment 1861 or 1862, wherein the oligomeric compound modulates the amount or activity of a cancer cell target.
1871. The method of embodiment 1861 or 1862, wherein the oligomeric compound modulates the amount or activity of a tumor cell target.
1872. The method of embodiment 1861 or 1862, wherein the oligomeric compound modulates the amount or activity of a smooth muscle cell target.
1873. The method of embodiment 1861 or 1862, wherein the oligomeric compound modulates the amount or activity of a lymphocyte target.
1874. The method of embodiment 1861 or 1862, wherein the oligomeric compound modulates the amount or activity of a pulmonary cell target.
1875. The method of embodiment 1861 or 1862, wherein the oligomeric compound modulates the amount or activity of a heart muscle cell target.
1876. The method of embodiment 1861 or 1862, wherein the oligomeric compound modulates the amount or activity of a cardiomyocyte target.
1877. The method of embodiment 1861 or 1862, wherein the oligomeric compound modulates the amount or activity of a endothelial cell target.
1878. The method of embodiment 1861 or 1862, wherein the oligomeric compound modulates the amount or activity of a fibroblast target.
1879. The method of embodiment 1861 or 1862, wherein the oligomeric compound modulates the amount or activity of a glial cell target.
1880. The method of embodiment 1861 or 1862, wherein the oligomeric compound modulates the amount or activity of a Schwann cell target.
1881. The method of embodiment 1861 or 1862, wherein the oligomeric compound modulates the amount or activity of a pancreatic cell target.
1882. The method of embodiment 1861 or 1862, wherein the oligomeric compound modulates the amount or activity of a kidney cell target.
1883. The method of embodiment 1861 or 1862, wherein the oligomeric compound modulates the amount or activity of a beta cell target.
1884. The method of embodiment 1861 or 1862, wherein the oligomeric compound modulates the amount or activity of a non-parenchymal cell target.
1885. The method of embodiment 1861 or 1862, wherein the oligomeric compound modulates the amount or activity of a hepatocyte target.
1886. The method of embodiment 1861 or 1862, wherein the oligomeric compound modulates the amount or activity of a oligodendrocyte target.
1887. The method of embodiment 1861 or 1862, wherein the oligomeric compound modulates the amount or activity of a astrocyte target.
1888. The method of embodiment 1861 or 1862, wherein the oligomeric compound modulates the amount or activity of a microglia target.
1889. The method of embodiment 1861 or 1862, wherein the oligomeric compound modulates the amount or activity of a ependymal cell target.
1890. The method of embodiment 1861 or 1862, wherein the oligomeric compound modulates the amount or activity of a sensory neuron target.
1891. The method of embodiment 1861 or 1862, wherein the oligomeric compound modulates the amount or activity of a motor neuron target.
1892. The method of embodiment 1861 or 1862, wherein the oligomeric compound modulates the amount or activity of a skeletal muscle target.
1893. The method of embodiment 1861 or 1862, wherein the oligomeric compound modulates the amount or activity of a cardiac muscle target.
1894. The method of embodiment 1861 or 1862, wherein the oligomeric compound modulates the amount or activity of a smooth muscle target.
1895. The method of embodiment 1861 or 1862, wherein the oligomeric compound modulates the amount or activity of a adipose tissue target.
1896. The method of embodiment 1861 or 1862, wherein the oligomeric compound modulates the amount or activity of a white adipose tissue target.
1897. The method of embodiment 1861 or 1862, wherein the oligomeric compound modulates the amount or activity of a spleen target.
1898. The method of embodiment 1861 or 1862, wherein the oligomeric compound modulates the amount or activity of a bone target.
1899. The method of embodiment 1861 or 1862, wherein the oligomeric compound modulates the amount or activity of a bone marrow target.
1900. The method of embodiment 1861 or 1862, wherein the oligomeric compound modulates the amount or activity of an intestinal target.
1901. The method of embodiment 1861 or 1862, wherein the oligomeric compound modulates the amount or activity of an adrenal gland target.
1902. The method of embodiment 1861 or 1862, wherein the oligomeric compound modulates the amount or activity of a testicular target.
1903. The method of embodiment 1861 or 1862, wherein the oligomeric compound modulates the amount or activity of an ovarian target.
1904. The method of embodiment 1861 or 1862, wherein the oligomeric compound modulates the amount or activity of a pancreatic target.
1905. The method of embodiment 1861 or 1862, wherein the oligomeric compound modulates the amount or activity of a pituitary gland target.
1906. The method of embodiment 1861 or 1862, wherein the oligomeric compound modulates the amount or activity of a prostate gland target.
1907. The method of embodiment 1861 or 1862, wherein the oligomeric compound modulates the amount or activity of a skin target.
1908. The method of embodiment 1861 or 1862, wherein the oligomeric compound modulates the amount or activity of an epidermal target.
1909. The method of embodiment 1861 or 1862, wherein the oligomeric compound modulates the amount or activity of a uterine target.
1910. The method of embodiment 1861 or 1862, wherein the oligomeric compound modulates the amount or activity of a bladder target.
1911. The method of embodiment 1861 or 1862, wherein the oligomeric compound modulates the amount or activity of a brain target.
1912. The method of embodiment 1861 or 1862, wherein the oligomeric compound modulates the amount or activity of a glomerulus target.
1913. The method of embodiment 1861 or 1862, wherein the oligomeric compound modulates the amount or activity of a distal tubular epithelium target.
1914. The method of embodiment 1861 or 1862, wherein the oligomeric compound modulates the amount or activity of a breast tissue target.
1915. The method of embodiment 1861 or 1862, wherein the oligomeric compound modulates the amount or activity of a lung target.
1916. The method of embodiment 1861 or 1862, wherein the oligomeric compound modulates the amount or activity of a heart target.
1917. The method of embodiment 1861 or 1862, wherein the oligomeric compound modulates the amount or activity of a kidney target.
1918. The method of embodiment 1861 or 1862, wherein the oligomeric compound modulates the amount or activity of a ganglion target.
1919. The method of embodiment 1861 or 1862, wherein the oligomeric compound modulates the amount or activity of a frontal cortex target.
1920. The method of embodiment 1861 or 1862, wherein the oligomeric compound modulates the amount or activity of a spinal cord target.
1921. The method of embodiment 1861 or 1862, wherein the oligomeric compound modulates the amount or activity of a trigeminal ganglion target.
1922. The method of embodiment 1861 or 1862, wherein the oligomeric compound modulates the amount or activity of a sciatic nerve target.
1923. The method of embodiment 1861 or 1862, wherein the oligomeric compound modulates the amount or activity of a dorsal root ganglion target.
1924. The method of embodiment 1861 or 1862, wherein the oligomeric compound modulates the amount or activity of a epidymal fat target.
1925. The method of embodiment 1861 or 1862, wherein the oligomeric compound modulates the amount or activity of a diaphragm target.
1926. The method of embodiment 1861 or 1862, wherein the oligomeric compound modulates the amount or activity of a colon target.
1927. The method of any of embodiments 1802-1926, wherein the plasma ALT level of the human subject after the administration is not more than three times higher than it was prior to the administration.
1928. The method of any of embodiments 1802-1926, wherein the plasma ALT level of the human subject after the administration is not more than 150 units per liter.
1929. The method of any of embodiments 1802-1928, wherein the human subject is susceptible to liver damage.
1930. The method of any of embodiments 1802-1928, wherein the human subject is susceptible to liver degeneration.
1931. The method of any of embodiments 1802-1930, wherein the human subject is susceptible to elevated apoptosis in the liver.
1932. The method of any of embodiments 1802-1931, wherein the human subject has a liver disease.
1933. The method of any of embodiments 1802-1932, wherein the human subject has kidney disease.
1934. The method of any of embodiments 1802-1933, wherein the human subject is susceptible to kidney damage.
1935. The method of any of embodiments 1802-1934, wherein the human subject has heart disease.
1936. The method of any of embodiments 1802-1935, wherein the human subject is susceptible to heart damage.
1937. The method of any of embodiments 1802-1936, wherein the human subject has pancreatitis.
1938. The method of any of embodiments 1802-1937, wherein the human subject is susceptible to pancreatic damage.
1939. The method of any of embodiments 1802-1938, wherein the human subject has a neurological disease.
1940. The method of any of embodiments 1802-1939, wherein the human subject is susceptible to neurological damage.
1941. The method of any of embodiments 1859-1940, comprising administering the parent oligomeric compound of the oligomeric compound of any of embodiments 1461-1858 to a mouse.
1942. The method of any of embodiments 1859-1940, comprising contacting a cell with the parent oligomeric compound of the oligomeric compound of any of embodiments 1461-1858.
1943. The method of embodiment 1941 or 1942, wherein each sugar moiety of the central region of the parent oligomeric compound is an unmodified 2′-β-D-deoxyribosyl sugar moiety, and wherein each internucleoside linkage of the central region of the parent oligomeric compound is a phosphorothioate internucleoside linkage.
1944. A method of designing an oligomeric compound comprising starting with a parent oligomeric compound and changing the design of that compound in order to arrive at an oligomeric compound of any one of embodiments 1461-1858.
1945. A method of designing an oligomeric compound comprising identifying a parent oligomeric compound and changing the design of that parent oligomeric compound to arrive at a second oligomeric compound, wherein the second oligomeric compound is an oligomeric compound of any one of embodiments 1461-1858.
1946. A method of improving hepatotoxicity of an oligomeric compound comprising the steps of (i) identifying a parent oligomeric compound that has plasma ALT levels above 300 units per liter in a mouse, and (ii) providing an oligomeric compound according to any one of embodiments 1461-1858.
1947. The method of embodiment 1945 or 1946, wherein the method designs an oligomeric compound with improved therapeutic index relative to the parent oligomeric compound.
1948. The method of embodiment 1945 or 1946, wherein the method designs an oligomeric compound with lower hepatotoxicity relative to the parent oligomeric compound.
1949. The method of embodiment 1945, wherein the second oligomeric compound has an improved therapeutic index relative to the parent oligomeric compound.
1950. The method of embodiment 1945 or 1946, wherein the second oligomeric compound has reduced hepatotoxicity in a mouse relative to the parent oligomeric compound.
1951. The method of embodiment 1945, wherein the oligomeric compound according to any one of embodiments 1461-1858 has reduced hepatotoxicity relative to the parent oligomeric compound.
1952. A method comprising administering an oligomeric compound of any of embodiments 1461-1858 to a mouse and separately administering the parent oligomeric compound of the oligomeric compound of any of embodiments 1461-1858 to a second mouse, wherein the therapeutic index of the oligomeric compound of any of embodiments 1461-1858 is improved relative to the therapeutic index of the parent oligomeric compound.
1953. The method of any of embodiments 1859-1952, wherein the level of p21 RNA is measured in the mouse, the cell, and/or the human subject.
1954. A method comprising administering an oligomeric compound of any of embodiments 1461-1858 to a subject and measuring the level of p21 RNA in the subject.
1955. The method of embodiment 1954, wherein the subject is a mouse.
1956. The method of embodiment 1954, wherein the subject is a human.
1957. The method of any of embodiments 1954-1956, wherein the p21 RNA level is measured within 24 hours of the administration.
1958. The method of any of embodiments 1954-1956, wherein the p21 RNA level is measured 24-48 hours following the administration.
1959. An oligomeric compound or composition of any one of embodiments 1461-1858, for use in medical therapy.
1960. The oligomeric compound of any of embodiments 1461-1858, wherein the oligomeric compound is not toxic.
1961. The oligomeric compound of any of embodiment 1461-1858, wherein a comparator compound is toxic; wherein the comparator compound is identical to the oligomeric compound except that each nucleoside of the central region of the comparator compound is a nucleoside comprising 2′-β-D-deoxyribosyl sugar moiety.
1962. The oligomeric compound of embodiment 1961, wherein administering a dose of the oligomeric compound to an animal results in a circulating ALT level that is at least 3-fold lower than the ALT level that results from administering the same dose of the comparator compound to an animal for at least one matched dose.
1963. The oligomeric compound of embodiment 1961 or 1962, wherein the lowest dose of the oligomeric compound that raises circulating ALT to over 300 units per liter is at least three-fold higher than the lowest dose of the comparator compound that raises circulating ALT to over 300 units per liter.
1964. The oligomeric compound of any of embodiments 1961-1963, wherein the oligomeric compound is capable of reducing the target RNA in a cell.
1965. The oligomeric compound of embodiment 1964, wherein the oligomeric compound has RNA-reducing activity comparable to a comparator compound, wherein the comparator compound is identical to the oligomeric compound except that each nucleoside of the central region of the comparator compound is a nucleoside comprising 2′-β-D-deoxyribosyl sugar moiety.
1966. The oligomeric compound of embodiment 1965 having RNA-reducing activity that is no more than 2-fold worse than the RNA-reducing activity of the comparator compound.
1967. A method of modulating the amount or activity of a target RNA in a human subject comprising administering the oligomeric compound or composition of any of embodiments 1461-1858 to the human subject; and thereby modulating the amount or activity of the target RNA in the human subject.
1968. A method of treating a disease or disorder in a human subject comprising administering the oligomeric compound or composition of any of embodiments 1461-1858 to the human subject having the disease or disorder; and thereby treating the disease or disorder in the human subject.
1969. The method of embodiment 1967 or 1968, wherein the disease or disorder is a liver disease, and wherein the oligomeric compound modulates the amount or activity of a liver target RNA.
1970. The method of embodiment 1967 or 1968, wherein the disease or disorder is a central nervous system disease, and wherein the oligomeric compound modulates the amount or activity of a central nervous system target RNA.
1971. The method of any of embodiments 1967-1970, wherein the disease or disorder is a cancer, a neurodegenerative disease, a cardiovascular disease, an immunological disease, a hereditary disease, a repeat expansion disease, a muscle-wasting disease, or a metabolic disease.
1972. The method of any of embodiments 1967-1971, wherein the plasma ALT level of the human subject after the administration is not more than three times higher than it was prior to the administration.
1973. The method of any of embodiments 1967-1972, wherein the plasma ALT level of the human subject after the administration is not more than 150 units per liter.
1974. A method of treating a disease or disorder in a human subject comprising administering the oligomeric compound or composition of any of embodiments 1461-1858 to the human subject having the disease or disorder; and thereby treating the disease or disorder in the human subject.
1975. The method of embodiment 1974, wherein the disease or disorder is not a CNS disease or disorder.
1976. The method of embodiment 1974, wherein the disease or disorder is a disease or disorder of the white fat cells.
1977. The method of embodiment 1974, wherein the disease or disorder is a disease or disorder of the brown fat cells.
1978. The method of embodiment 1974, wherein the disease or disorder is a disease or disorder of the adipocytes.
1979. The method of embodiment 1974, wherein the disease or disorder is a disease or disorder of the macrophages.
1980. The method of embodiment 1974, wherein the disease or disorder is a disease or disorder of the cancer cells.
1981. The method of embodiment 1974, wherein the disease or disorder is a disease or disorder of the tumor cells.
1982. The method of embodiment 1974, wherein the disease or disorder is a disease or disorder of the smooth muscle cells.
1983. The method of embodiment 1974, wherein the disease or disorder is a disease or disorder of the lymphocytes.
1984. The method of embodiment 1974, wherein the disease or disorder is a disease or disorder of the pulmonary cells.
1985. The method of embodiment 1974, wherein the disease or disorder is a disease or disorder of the heart muscle cells.
1986. The method of embodiment 1974, wherein the disease or disorder is a disease or disorder of the cardiomyocytes.
1987. The method of embodiment 1974, wherein the disease or disorder is a disease or disorder of the endothelial cells.
1988. The method of embodiment 1974, wherein the disease or disorder is a disease or disorder of the fibroblasts.
1989. The method of embodiment 1974, wherein the disease or disorder is a disease or disorder of the glial cells.
1990. The method of embodiment 1974, wherein the disease or disorder is a disease or disorder of the Schwann cells.
1991. The method of embodiment 1974, wherein the disease or disorder is a disease or disorder of the pancreatic cells.
1992. The method of embodiment 1974, wherein the disease or disorder is a disease or disorder of the kidney cells.
1993. The method of embodiment 1974, wherein the disease or disorder is a disease or disorder of the beta cells.
1994. The method of embodiment 1974, wherein the disease or disorder is a disease or disorder of the non-parenchymal cells.
1995. The method of embodiment 1974, wherein the disease or disorder is a disease or disorder of the hepatocytes.
1996. The method of embodiment 1974, wherein the disease or disorder is a disease or disorder of the oligodendrocytes.
1997. The method of embodiment 1974, wherein the disease or disorder is a disease or disorder of the astrocytes.
1998. The method of embodiment 1974, wherein the disease or disorder is a disease or disorder of the microglia.
1999. The method of embodiment 1974, wherein the disease or disorder is a disease or disorder of the ependymal cells.
2000. The method of embodiment 1974, wherein the disease or disorder is a disease or disorder of the sensory neurons.
2001. The method of embodiment 1974, wherein the disease or disorder is a disease or disorder of the motor neurons.
2002. The method of embodiment 1974, wherein the disease or disorder is a disease or disorder of the skeletal muscle.
2003. The method of embodiment 1974, wherein the disease or disorder is a disease or disorder of the cardiac muscle.
2004. The method of embodiment 1974, wherein the disease or disorder is a disease or disorder of the smooth muscle.
2005. The method of embodiment 1974, wherein the disease or disorder is a disease or disorder of the adipose tissue.
2006. The method of embodiment 1974, wherein the disease or disorder is a disease or disorder of the white adipose tissue.
2007. The method of embodiment 1974, wherein the disease or disorder is a disease or disorder of the spleen.
2008. The method of embodiment 1974, wherein the disease or disorder is a disease or disorder of the bone.
2009. The method of embodiment 1974, wherein the disease or disorder is a disease or disorder of the bone marrow.
2010. The method of embodiment 1974, wherein the disease or disorder is a disease or disorder of the intestine.
2011. The method of embodiment 1974, wherein the disease or disorder is a disease or disorder of the adrenal glands.
2012. The method of embodiment 1974, wherein the disease or disorder is a disease or disorder of the testes.
2013. The method of embodiment 1974, wherein the disease or disorder is a disease or disorder of the ovaries.
2014. The method of embodiment 1974, wherein the disease or disorder is a disease or disorder of the pancreas.
2015. The method of embodiment 1974, wherein the disease or disorder is a disease or disorder of the pituitary gland.
2016. The method of embodiment 1974, wherein the disease or disorder is a disease or disorder of the prostate gland.
2017. The method of embodiment 1974, wherein the disease or disorder is a disease or disorder of the skin.
2018. The method of embodiment 1974, wherein the disease or disorder is a disease or disorder of the epidermis.
2019. The method of embodiment 1974, wherein the disease or disorder is a disease or disorder of the uterus.
2020. The method of embodiment 1974, wherein the disease or disorder is a disease or disorder of the bladder.
2021. The method of embodiment 1974, wherein the disease or disorder is a disease or disorder of the brain.
2022. The method of embodiment 1974, wherein the disease or disorder is a disease or disorder of the glomerulus.
2023. The method of embodiment 1974, wherein the disease or disorder is a disease or disorder of the distal tubular epithelium.
2024. The method of embodiment 1974, wherein the disease or disorder is a disease or disorder of the breast tissue.
2025. The method of embodiment 1974, wherein the disease or disorder is a disease or disorder of the lung.
2026. The method of embodiment 1974, wherein the disease or disorder is a disease or disorder of the heart.
2027. The method of embodiment 1974, wherein the disease or disorder is a disease or disorder of the kidney.
2028. The method of embodiment 1974, wherein the disease or disorder is a disease or disorder of the ganglion.
2029. The method of embodiment 1974, wherein the disease or disorder is a disease or disorder of the frontal cortex.
2030. The method of embodiment 1974, wherein the disease or disorder is a disease or disorder of the spinal cord.
2031. The method of embodiment 1974, wherein the disease or disorder is a disease or disorder of the trigeminal ganglion.
2032. The method of embodiment 1974, wherein the disease or disorder is a disease or disorder of the sciatic nerve.
2033. The method of embodiment 1974, wherein the disease or disorder is a disease or disorder of the dorsal root ganglion.
2034. The method of embodiment 1974, wherein the disease or disorder is a disease or disorder of the epidymal fat.
2035. The method of embodiment 1974, wherein the disease or disorder is a disease or disorder of the diaphragm.
2036. The method of embodiment 1974, wherein the disease or disorder is a disease or disorder of the colon.
2037. A method of screening a library of oligomeric compounds for activity against a target RNA, wherein the library of oligomeric compounds comprises a plurality of oligomeric compounds of any of embodiments 1461-1858.

### Certain Compounds

In certain embodiments, compounds described herein are oligomeric compounds comprising or consisting of oligonucleotides consisting of linked nucleosides. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety and/or a modified nucleobase) and/or at least one modified internucleoside linkage).

### I. Modifications

### A. Modified Nucleosides

Modified nucleosides comprise a modified sugar moiety, a modified nucleobase, or both a modified sugar moiety and a modified nucleobase.

### 1. Certain Modified Sugar Moieties

In certain embodiments, sugar moieties are non-bicyclic, modified furanosyl sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments, the furanosyl sugar moiety is a β-D-ribofuranosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“2′-OMe” or “2′-O-methyl”), and 2′-O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted nucleoside or non-bicyclic 2′-modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or non-bicyclic 2′-modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or non-bicyclic 2′-modified nucleoside comprises a sugar moiety comprising a non-bridging 2′-substituent group selected from: F, OCH₃, and OCH₂CH₂OCH₃.

In certain embodiments, the 4′ 0 of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

Certain modified sugar moieties comprise a bridging sugar substituent that forms a second ring resulting in a bicyclic sugar moiety. In certain such embodiments, the bicyclic sugar moiety comprises abridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂-O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem.,2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672), 4′-C(═O)—N(CH₃)2-2′, 4′-C(═O)—N(R)₂-2′, 4′-C(═S)—N(R)₂-2′ and analgos thereof (see, e.g., Obika et al., WO2011052436A1, Yusuke, WO2017018360A1).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J. Am. Chem. Soc., 2017, 129, 8362-8379; Elayadi et al.,; Christiansen, et al., J. Am. Chem. Soc. 1998, 120, 5458-5463; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

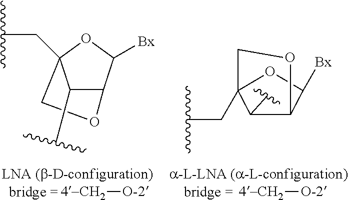

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

Nucleosides comprising modified furanosyl sugar moieties and modified furanosyl sugar moieties may be referred to by the position(s) of the substitution(s) on the sugar moiety of the nucleoside. The term “modified” following a position of the furanosyl ring, such as “2′-modified”, indicates that the sugar moiety comprises the indicated modification at the 2′ position and may comprise additional modifications and/or substituents. A 4′-2′ bridged sugar moiety is 2′-modified and 4′-modified, or, alternatively, “2′, 4′-modified”. The term “substituted” following a position of the furanosyl ring, such as “2′-substituted” or “2′-4′-substituted”, indicates that is the only position(s) having a substituent other than those found in unmodified sugar moieties in oligonucleotides. Accordingly, the following sugar moieties are represented by the following formulas.

In the context of a nucleoside and/or an oligonucleotide, a non-bicyclic, modified furanosyl sugar moiety is represented by formula I.

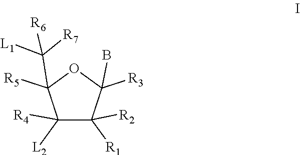

wherein B is a nucleobase; and L₁ and L₂ are each, independently, an internucleoside linkage, a terminal group, a conjugate group, or a hydroxyl group. Among the R groups, at least one of R₃₋₇ is not H and/or at least one of R₁ and R₂ is not H or OH. In a 2′-modified furanosyl sugar moiety, at least one of R₁ and R₂ is not H or OH and each of R₃₋₇ is independently selected from H or a substituent other than H. In a 4′-modified furanosyl sugar moiety, R₅ is not H and each of R_{1-4, 6, 7} are independently selected from H and a substituent other than H; and so on for each position of the furanosyl ring. The stereochemistry is not defined unless otherwise noted.

In the context of a nucleoside and/or an oligonucleotide, a non-bicyclic, modified, substituted fuarnosyl sugar moiety is represented by formula I, wherein B is a nucleobase; and L₁ and L₂ are each, independently, an internucleoside linkage, a terminal group, a conjugate group, or a hydroxyl group. Among the R groups, either one (and no more than one) of R₃₋₇ is a substituent other than H or one of R₁ or R₂ is a substituent other than H or OH. The stereochemistry is not defined unless otherwise noted. Examples of non-bicyclic, modified, substituted furanosyl sugar moieties include 2′-substituted ribosyl, 4′-substituted ribosyl, and 5′-substituted ribosyl sugar moieties, as well as substituted 2′-deoxyfuranosyl sugar moieties, such as 4′-substituted 2′-deoxyribosyl and 5′-substituted 2′-deoxyribosyl sugar moieties.

In the context of a nucleoside and/or an oligonucleotide, a 2′-substituted ribosyl sugar moiety is represented by formula II:

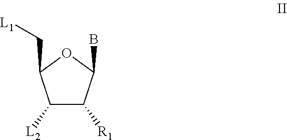

wherein B is a nucleobase; and L₁ and L₂ are each, independently, an internucleoside linkage, a terminal group, a conjugate group, or a hydroxyl group. R₁ is a substituent other than H or OH. The stereochemistry is defined as shown.

In the context of a nucleoside and/or an oligonucleotide, a 4′-substituted ribosyl sugar moiety is represented by formula III:

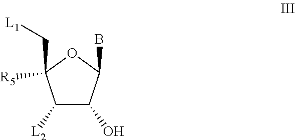

wherein B is a nucleobase; and L₁ and L₂ are each, independently, an internucleoside linkage, a terminal group, a conjugate group, or a hydroxyl group. R₅ is a substituent other than H. The stereochemistry is defined as shown.

In the context of a nucleoside and/or an oligonucleotide, a 5′-substituted ribosyl sugar moiety is represented by formula IV:

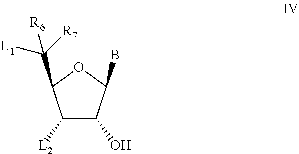

wherein B is a nucleobase; and L₁ and L₂ are each, independently, an internucleoside linkage, a terminal group, a conjugate group, or a hydroxyl group. R₆ or R₇ is a substituent other than H. The stereochemistry is defined as shown.

In the context of a nucleoside and/or an oligonucleotide, a 2′-deoxyfuranosyl sugar moiety is represented by formula V:

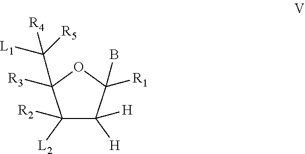

wherein B is a nucleobase; and L₁ and L₂ are each, independently, an internucleoside linkage, a terminal group, a conjugate group, or a hydroxyl group. Each of R₁₋₅ are independently selected from H and a non-H substituent. If all of R₁₋₅ are each H, the sugar moiety is an unsubstituted 2′-deoxyfuranosyl sugar moiety. The stereochemistry is not defined unless otherwise noted.

In the context of a nucleoside and/or an oligonucleotide, a 4′-substituted 2′-deoxyribosyl sugar moiety is represented by formula VI:

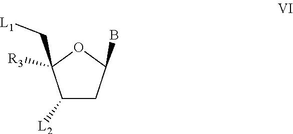

wherein B is a nucleobase; and L₁ and L₂ are each, independently, an internucleoside linkage, a terminal group, a conjugate group, or a hydroxyl group. R₃ is a substituent other than H. The stereochemistry is defined as shown.

In the context of a nucleoside and/or an oligonucleotide, a 5′-substituted 2′-deoxyribosyl sugar moiety is represented by formula VII:

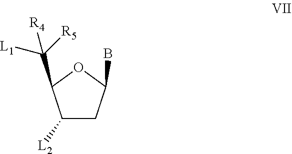

wherein B is a nucleobase; and L₁ and L₂ are each, independently, an internucleoside linkage, a terminal group, a conjugate group, or a hydroxyl group. R₄ or R₅ is a substituent other than H. The stereochemistry is defined as shown.

Unsubstituted 2′-deoxyfuranosyl sugar moieties may be unmodified (β-D-2′-deoxyribosyl) or modified. Examples of modified, unsubstituted 2′-deoxyfuranosyl sugar moieties include 3-L-2′-deoxyribosyl, α-L-2′-deoxyribosyl, α-D-2′-deoxyribosyl, and β-D-xylosyl sugar moieties. For example, in the context of a nucleoside and/or an oligonucleotide, a β-L-2′-deoxyribosyl sugar moiety is represented by formula VIII:

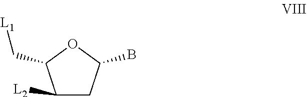

wherein B is a nucleobase; and L₁ and L₂ are each, independently, an internucleoside linkage, a terminal group, a conjugate group, or a hydroxyl group. The stereochemistry is defined as shown. Synthesis of α-L-ribosyl nucleotides and β-D-xylosyl nucleotides has been described by Gaubert, et al., Tetehedron 2006, 62: 2278-2294. Additional isomers of DNA and RNA nucleosides are described by Vester, et. al., “Chemically modified oligonucleotides with efficient RNase H response,” Bioorg. Med. Chem. Letters, 2008, 18: 2296-2300.

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, CJ. Bioorg. & Med. Chem. 2002, 10, 841-854), fluoro HNA

(“F-HNA”, see e.g.Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), F-CeNA, and 3′-ara-HNA, having the formulas below, where L₁ and L₂ are each, independently, an internucleoside linkage linking the modified THP nucleoside to the remainder of an oligonucleotide or one of L₁ and L₂ is an internucleoside linkage linking the modified THP nucleoside to the remainder of an oligonucleotide and the other of L₁ and L₂ is H, a hydroxyl protecting group, a linked conjugate group, or a 5′ or 3′-terminal group.

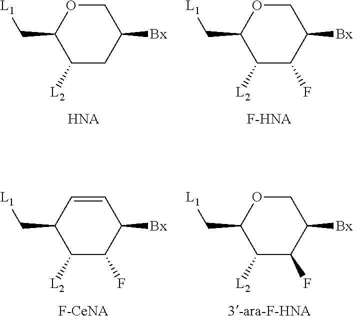

Additional sugar surrogates comprise THP compounds having the formula:

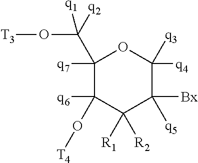

wherein, independently, for each of said modified THP nucleoside:
Bx is a nucleobase moiety;
T₃ and T₄ are each, independently, an internucleoside linkage linking the modified THP nucleoside to the remainder of an oligonucleotide or one of T₃ and T₄ is an internucleoside linkage linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

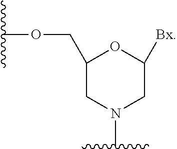

In certain embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

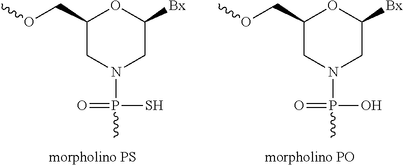

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876.

Many other bicyclic and tricyclic sugar and sugar surrogate ring systems are known in the art that can be used in modified nucleosides. Certain such ring systems are described in Hanessian, et al., J. Org. Chem., 2013, 78: 9051-9063 and include bcDNA and tcDNA. Modifications to bcDNA and tcDNA, such as 6′-fluoro, have also been described (Dogovic and Leumann, J. Org. Chem., 2014, 79: 1271-1279).

In certain embodiments, modified nucleosides are DNA mimics. “DNA mimic” means a nucleoside other than a DNA nucleoside wherein the nucleobase is directly linked to a carbon atom of a ring bound to a second carbon atom within the ring, wherein the second carbon atom comprises a bond to at least one hydrogen atom, wherein the nucleobase and at least one hydrogen atom are trans to one another relative to the bond between the two carbon atoms.

In certain embodiments, a DNA mimic comprises a structure represented by the formula below:

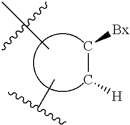

Wherein Bx represents a heterocyclic base moiety.
In certain embodiments, a DNA mimic comprises a structure represented by one of the formulas below:

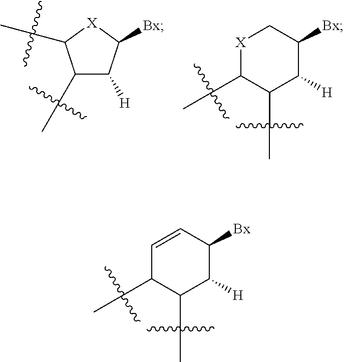

wherein X is O or S and Bx represents a heterocyclic base moiety.

In certain embodiments, a DNA mimic is a sugar surrogate. In certain embodiments, a DNA mimic is a cycohexenyl or hexitol nucleic acid. In certain embodiments, a DNA mimic is described in FIG. 1 of Vester, et. al., “Chemically modified oligonucleotides with efficient RNase H response,” Bioorg. Med. Chem. Letters, 2008, 18: 2296-2300, incorporated by reference herein. In certain embodiments, a DNA mimic nucleoside has a formula selected from:

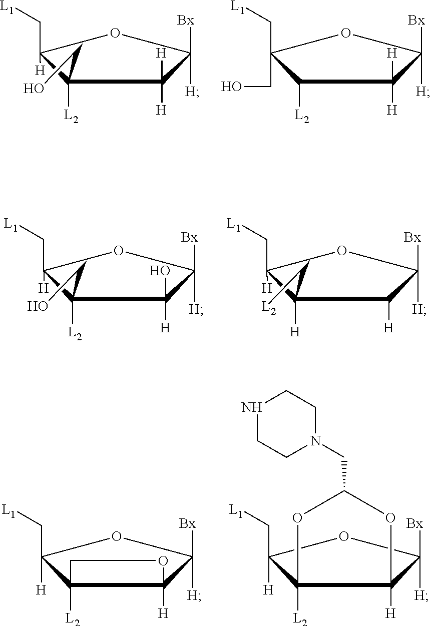

wherein Bx is a heterocyclic base moiety, and L₁ and L₂ are each, independently, an internucleoside linkage linking the modified THP nucleoside to the remainder of an oligonucleotide or one of L₁ and L₂ is an internucleoside linkage linking the modified nucleoside to the remainder of an oligonucleotide and the other of L₁ and L₂ is H, a hydroxyl protecting group, a linked conjugate group, or a 5′ or 3′-terminal group. In certain embodiments, a DNA mimic is α,β-constrained nucleic acid (CAN), 2′,4′-carbocyclic-LNA, or 2′,4′-carbocyclic-ENA. In certain embodiments, a DNA mimic has a sugar moiety selected from among: 4′-C-hydroxymethyl-2′-deoxyribosyl, 3′-C-hydroxymethyl-2′-deoxyribosyl, 3′-C-hydroxymethyl-arabinosyl, 3′-C-2′-O-arabinosyl, 3′-C-methylene-extended-xyolosyl, 3′-C-2′-O-piperazino-arabinosyl. In certain embodiments, a DNA mimic has a sugar moiety selected from among: 2′-methylribosyl, 2′-S-methylribosyl, 2′-aminoribosyl, 2′-NH(CH₂)-ribosyl, 2′-NH(CH₂)₂-ribosyl, 2′-CH2-F-ribosyl, 2′-CHF2-ribosyl, 2′-CF3-ribosyl, 2′=CF₂ ribosyl, 2′-ethylribosyl, 2′-alkenylribosyl, 2′-alkynylribosyl, 2′-O-4′-C-methyleneribosyl, 2′-cyanoarabinosyl, 2′-chloroarabinosyl, 2′-fluoroarabinosyl, 2′-bromoarabinosyl, 2′-azidoarabinosyl, 2′-methoxyarabinosyl, and 2′-arabinosyl. In certain embodiments, a DNA mimic has a sugar moiety selected from 4′-methyl-modified deoxyfuranosyl, 4′-F-deoxyfuranosyl, 4′-OMe-deoxyfuranosyl. In certain embodiments, a DNA mimic has a sugar moiety selected from among: 5′-methyl-2′-β-D-deoxyribosyl, 5′-ethyl-2′-β-D-deoxyribosyl, 5′-allyl-2′-β-D-deoxyribosyl, 2′-fluoro-β-D-arabinofuranosyl. In certain embodiments, DNA mimics are listed on page 32-33 of PCT/US00/267929 as B-form nucleotides, incorporated by reference herein in its entirety.

### 2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinaset al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to an target nucleic acid comprising one or more modified nucleobases. In certain embodiments, the modified nucleobase is 5-methylcytosine. In certain embodiments, each cytosine is a 5-methylcytosine.

### B. Modified Internucleoside Linkages

In certain embodiments, compounds described herein having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS-P=S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

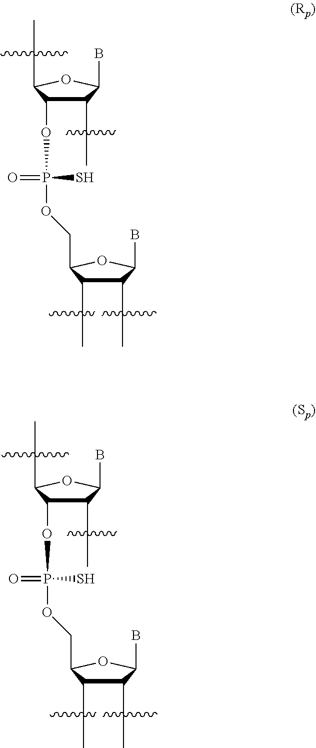

Unless otherwise indicated, chiral internucleoside linkages of modified oligonucleotides described herein can be stereorandom or in a particular stereochemical configuration.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′- O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

In certain embodiments, nucleic acids can be linked 2′ to 5′ rather than the standard 3′ to 5′ linkage. Such a linkage is illustrated below.

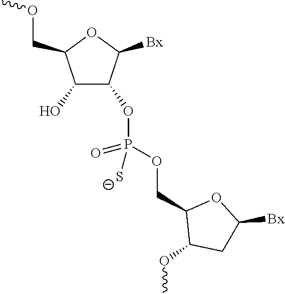

In the context of a nucleoside and/or an oligonucleotide, a non-bicyclic, 2′-linked modified furanosyl sugar moiety is represented by formula IX:

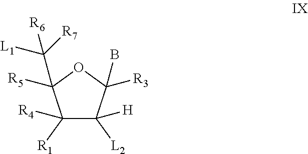

wherein B is a nucleobase; L₁ is an internucleoside linkage, a terminal group, a conjugate group, or a hydroxyl group and L₂ is an internucleoside linkage. The stereochemistry is not defined unless otherwise noted.

In certain embodiments, nucleosides can be linked by vinicinal 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

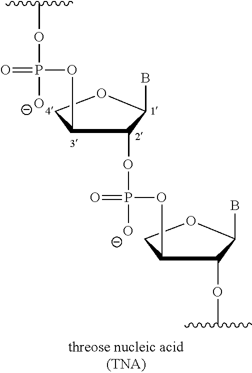

Additional modified linkages include α,β-D-CNA type linkages and related conformationally-constrained linkages, shown below. Synthesis of such molecules has been described previously (see Dupouy, et al., Angew. Chem. Int. Ed. Engl. 2014, 45: 3623-3627: Borsting, et al. Tetahedron, 2004, 60:10955-10966; Ostergaard, et al., ACS Chem. Biol. 2014, 9: 1975-1979; Dupouy, et al, Eur. J. Org. Chem., 2008, 1285-1294; Martinez, et al., PLoS One, 2011, 6:e25510; Dupouy, et al., Eur. J. Org. Chem. 2007, 5256-5264; Boissonnet, et al., New J. Chem., 2011, 35: 1528-1533.)

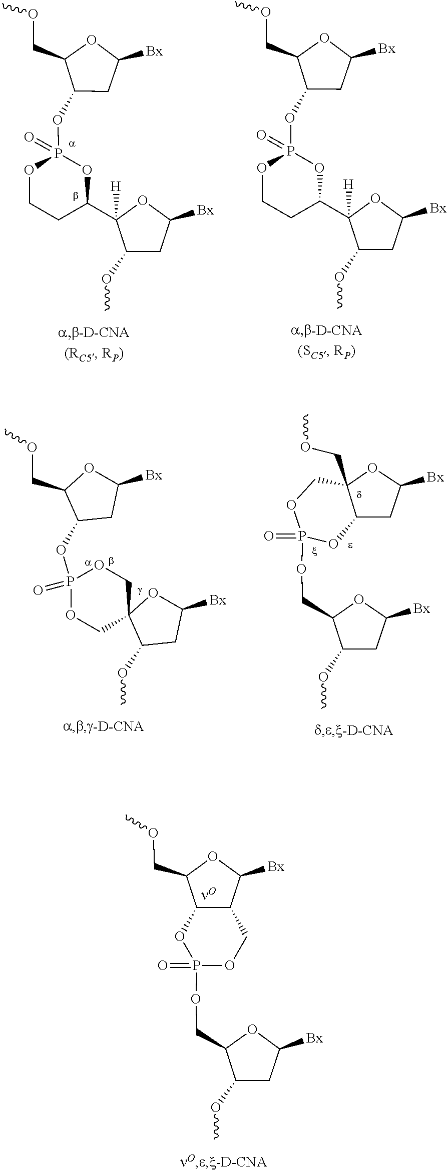

### II. Certain Motifs

In certain embodiments, oligomeric compounds described herein comprise or consist of oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### A. Certain Sugar Motifs

In certain embodiments, oligomeric compounds described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, a modified oligonucleotide comprises or consists of a gapmer. The sugar motif of a gapmer defines the regions of the gapmer: 5′-region, central region, and 3′-region. The positions of the nucleosides within each region are counted beginning at the 5′-end of each region. Each region of a gapmer is connected by an internucleoside linkage, as are the nucleosides within each region. Each nucleoside of the 5′-region and each nucleoside of the 3′-region comprise a 2′-modified furanosyl sugar moiety. The nucleoside at the first position (position 1) of the central region and the nucleoside at the last position of the central region are adjacent to the 5′-region and 3′-region, respectively, and each comprise a sugar moiety independently selected from a 2′-deoxyfuranosyl sugar moiety or a sugar surrogate. Unlike the nucleosides at the first and last positions of the central region, the nucleosides at the other positions within the central region may comprise a 2′-modified furanosyl sugar moiety. In certain embodiments, the 2′-modified furanosyl sugar moiety in the 5′ and 3′-regions is a 4′-2′-bicyclic sugar moiety. In certain embodiments, the 2′-modified furanosyl sugar moiety in the 5′ and 3′ regions is a cEt. In certain embodiments, the 2′-modified furanosyl sugar moiety is a 2′-MOE furanosyl sugar moiety. In certain embodiments, each nucleoside within the gap supports RNase H cleavage. In certain embodiments,a plurality of nucleosides within the gap support RNase H cleavage. In certain embodiments, the nucleoside at the first and last positions of the central region adjacent to the 5′ and 3′ regions are DNA nucleosides.

Herein, the lengths (number of nucleosides) of the three regions of a gapmer may be provided using the notation [# of nucleosides in the 5′-wing]−[# of nucleosides in the gap]−[# of nucleosides in the 3′-wing]. Thus, a 3-10-3 gapmer consists of 3 linked nucleosides in each wing and 10 linked nucleosides in the gap. Where such nomenclature is followed by a specific modification, that modification is the modification of each sugar moiety of each wing and the gap nucleosides comprise 2′-β-D-deoxyribosyl sugar moieties. Thus, a 5-10-5 MOE gapmer consists of 5 linked nucleosides comprising 2′-MOE-β-D-ribofuranosyl sugar moieties in the 5′-wing, 10 linked nucleosides comprising a 2′-β-D-deoxyribosyl sugar moiety in the gap, and 5 linked nucleosides comprising 2′-MOE-β-D-ribofuranosyl sugar moieties in the 3′-wing. A 3-10-3 cEt gapmer consists of 3 linked nucleosides comprising a cEt in the 5′-wing, 10 linked nucleosides comprising a 2′-β-D-deoxyribosyl sugar moiety in the gap, and 3 linked nucleosides comprising a cEt in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

The sugar motif of a 3-10-3 cEt gapmer may also be denoted by the notation kkk-d(10)-kkk, wherein each “k” represents a cEt and each “d” represents a 2′-β-D-deoxyribosyl sugar moiety. This sugar motif is independent of the nucleobase sequence, the internucleoside linkage motif, and any nucleobase modifications. A 5-10-5 MOE gapmer may be denoted by the notation eeeee-d(10)-eeeee or e(5)-d(10)-e(5), wherein each “e” represents a 2′-MOE-β-D-ribofuranosyl sugar moiety, and each “d” represents a 2′-β-D-deoxyribosyl sugar moiety.

### B. Certain Nucleobase Motifs

In certain embodiments, oligomeric compounds described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

### C. Certain Internucleoside Linkage Motifs

In certain embodiments, oligomeric compounds described herein comprise or consist of oligonucleotides. In certain embodiments, oligonucleotides comprise modified and/or unmodified internucleoside linkages arranged along the oligonucleotide or region thereof in a defined pattern or motif. In certain embodiments, each internucleoside linkage is a phosphodiester internucleoside linkage (P═O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, some or all of the internucleoside linkages in the 5′-region and 3′-region are unmodified phosphate linkages. In certain embodiments, the terminal internucleoside linkages are modified. In certain embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are phosphorothioate internucleoside linkages. In certain embodiments, all of the internucleoside linkages of the oligonucleotide are phosphorothioate internucleoside linkages. In certain embodiments, each internucleoside linkage of the oligonucleotide is selected from phosphodiester or phosphate and phosphorothioate. In certain embodiments, each internucleoside linkage of the oligonucleotide is selected from phosphodiester or phosphate and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, modified oligonucleotides comprise a linkage motif comprising all phosphorothioate linkages except for one or two methylphosphonate linkages. In certain embodiments, one methylphosphonate linkage is in the central region of an oligonucleotide.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

### III. Certain Modified Oligonucleotides

In certain embodiments, oligomeric compounds described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with a RNA-like nucleoside, including a nucleoside comprising a 2′-OMe modified sugar moiety, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### IV. Certain Conjugated Compounds

In certain embodiments, the oligomeric compounds described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, a conjugate linker is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the compound has been taken up, it is desirable that the conjugate group be cleaved to release the unconjugated oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

### 3. Certain Cell-Targeting Conjugate Moieties

In certain embodiments, a conjugate group comprises a cell-targeting conjugate moiety. In certain embodiments, a conjugate group has the general formula:

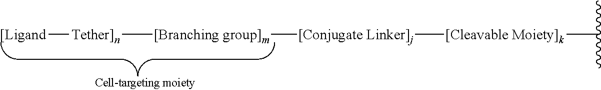

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-0-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Oligomeric compounds described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds or a salt thereof. In certain embodiments, the oligomeric compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

In certain embodiments, oligomeric compounds described herein comprise or consist of modified oligonucleotides. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Nucleosides that are sufficiently “DNA-like” to elicit RNase H activity are referred to as DNA mimics herein. Further, in certain embodiments, one or more non-DNA-like nucleoside in in the RNA:DNA duplex is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Certain Toxicities

For a compound to be a viable therapeutic agent, it must be safe at therapeutically relevant doses. It has become clear that toxicity of oligonucleotides can arise from one or more of several mechanisms. For example, some oligonucleotides hybridize an unintended RNA (or “off-target RNA”) resulting in reduction of the off-target RNA and the protein encoded by that off-target RNA. Such unintended protein reduction may have toxic consequences. The data disclosed herein demonstrate that toxicity can result from oligonucleotides binding certain proteins and subsequent sub-cellular localization of the oligonucleotide/protein complex. Other mechanisms of toxicity may also contribute. Of course, for an oligonucleotide to be a suitable drug for use in therapy, all of the forms or mechanisms of toxicity must be acceptably low.

Since toxicity can result from multiple mechanisms, the observed toxicity for a particular compound will typically be the most toxic mechanism or the mechanism that results in toxicity at the lowest dose for that particular compound (the “limiting toxicity”). Changes to a compound that reduce the limiting toxicity will result in a compound having an observable improvement in toxic profile. Changes that reduce a form of toxicity that is not the limiting toxicity may not result in an observable improvement in toxicity, because the improvement may be masked by the limiting toxicity. In such instances, the improvement to a non-limiting toxicity can nonetheless have value. For example, the limiting toxicity might be controlled through additional changes to the compound or through changes in dose or dose frequency or through use of a separate therapy that mitigates the limiting toxicity; at that point, a previously masked toxicity would become limiting. Alternatively, in certain circumstances, the limiting toxicity might be less relevant (for example, if the drug is intended for delivery to specific tissues not affected by the limiting toxicity or if the compound is for the treatment of severe or life-threatening indications where a certain degree of the limiting toxicity may be acceptable). In such instances, improvements to a non-limiting toxicity can have significant benefit. Further, the various forms and mechanisms of toxicity may have a cumulative effect, particularly over time. Accordingly, the beneficial effects of improvements to a particular mechanism of toxicity might be masked at an early time point where another mechanism is the limiting toxicity, but over time such masked toxicity may contribute or even predominate the overall safety profile.

In certain embodiments, oligomeric compounds of the invention have improved toxicity profiles compared to standard gapmer compounds having a gap comprising only nucleosides having 2′-β-D-deoxyribosyl sugar moieties and the same nucleobase sequence. It should be noted that some standard gapmers are suitable therapeutic agents. Toxicity is driven in part by nucleobase sequence (oligonucleotides having identical chemical modification patterns but different sequences can have vastly different safety profiles). When one attempts to modulate a particular target RNA, one might find an antisense oligonucleotide that has an acceptable safety profile at therapeutic doses. On other occasions, however, the most active/potent oligonucleotides have unacceptable toxicity. In such instances, it is desirable to modify such compounds to reduce their toxicity, ideally with no loss or only modest loss in activity/potency. In certain embodiments, modification motifs described herein reduce toxicity with little or no loss in activity/potency.

Without limitation to any particular mechanism, it is believed that certain modification motifs described herein may reduce interactions between an oligomeric compound and certain proteins. In certain embodiments, such interactions result in the limiting toxicity and so disruption of these interactions results in observable improvements in the toxicity profile. In certain embodiments, the motifs described herein may alter off-target cleavage. In certain embodiments, the disclosed motifs improve toxicity through an undefined mechanism. In certain embodiments, the motifs may improve toxicity through multiple mechanisms, including, but not limited to those described here.

Disclosed herein for comparison are certain gapmer oligonucleotides that are notably more toxic than other gapmers. These toxic gapmer oligonucleotides cause rapid delocalization of paraspeckle proteins, including p54nrb, to nucleoli, possibly due to the binding of these toxic oligonucleotides to the p54nrb protein and/or other paraspeckle proteins. Certain such toxic oligonucleotides have both more global protein binding than their nontoxic (or less toxic) counterparts and have higher binding affinities (i.e., lower Kd values) for key paraspeckle proteins, including p54nrb and RNase H1. Certain such toxic oligonucleotides, but not nontoxic (or less toxic) oligonucleotides, cause the paraspeckle proteins PSF, PSPC1, and FUS to localize to the nucleoli as well, as observed across a number of mouse and human cell types. In certain instances, the nucleolar delocalization of paraspeckle proteins is mediated by RNase H1. Importantly, in some instances, nontoxic (or less toxic) modified oligonucleotides with the same chemical modification pattern (gapmer motif) and a different sequence do not cause the delocalization of p54nrb to nucleoli. This early event leads to nucleolar stress, p53 activation, and apoptotic cell death, both in vitro across a number of mammalian cell types and in vivo in mice. These results were consistent for gapmers with toxic sequences and containing several different commonly-used chemical modifications of modified oligonucleotides, including LNA, cEt, and 2′-MOE.

In certain embodiments, the instant invention is directed towards modified oligonucleotides having chemical modifications that can alleviate the observed toxicity. In certain embodiments, such toxicity is related to protein binding and the resulting nucleolar mislocalization of proteins, such as paraspeckle proteins described above. In certain embodiments, the incorporation of a safety-enhancing nucleoside at position 2, 3 or 4 of the central region (or “gap”) of the modified oligonucleotide can reduce both global protein binding and the associated toxicity. In certain embodiments, the safety-enhancing nucleoside is a nucleoside comprising a 2′-OMe β-D-deoxyribosyl sugar moiety at position 2 of the central region. Incorporation of a nucleoside comprising a 2′-OMe-β-D-deoxyribosyl sugar moiety at position 2 of the central region in a toxic 3-10-3, 3-10-4, 4-10-3 cEt gapmer, a 3-10-3 LNA gapmer, a 5-10-5 MOE gapmer, or several cEt/MOE mixed wing gapmers reduced cellular toxicity and apoptosis in vitro and hepatoxicity in vivo across a wide variety of sequences, while having only a modest effect, if any, on antisense activity. In certain embodiments, incorporation of a nucleoside comprising a 2′-OMe-β-D-deoxyribosyl sugar moiety at position 2 of the central region also reduced delayed neurotoxicity, suggesting a common mechanism for delayed neurotoxicity and hepatoxicity. In certain embodiments, the safety-enhancing nucleoside is a nucleoside comprising a 5′-alkyl or 5′-allyl modified β-D-deoxyribosyl sugar moiety at position 3 or position 4 of the central region. In certain instances, incorporation of a nucleoside comprising a 5′-alkyl β-D-deoxyribosyl sugar moiety at position 3 or position 4 of the central region of a 3-10-3 cEt gapmer reduces cellular toxicity and apoptosis in vitro and hepatoxicity in vivo across a variety of sequences, while having a modest effect, if any, on antisense activity. In certain embodiments, the incorporation of a safety-enhancing internucleoside linkage between the nucleosides at positions 2-3 or positions 3-4 of the central region can reduce toxicity. In certain embodiments, one or more of a safety-enhancing linkages linking the nucleosides from positions 2-4 of the central region is a neutral linkage. In certain embodiments, one or more of a safety-enhancing linkages linking the nucleosides from positions 2-4 of the central region is a 2′-5′ internucleoside linkage. The instant invention extends to any chemical modification introduced at positions 2, 3 or 4 of the central region, including modifications to nucleosides and to internucleoside linkages. In certain embodiments, such modification reduces in vitro toxicity (compared to the modified oligonucleotide lacking the safety enhancing nucleoside at positions 2, 3 or 4 of the central region or lacking the safety enhancing internucleoside linkage between positions 2-3 or 3-4 of the central region) as measured by the caspase 3/7 assay and/or in vivo hepatotoxicity, as measured by ALT or AST, and/or in vivo neurotoxicity, as measured by an FOB score or accumulation of markers of glial inflammation, Aif1 and GFAP.

In certain embodiments, introducing chemical modifications at positions 2, 3 or 4 of the central region can significantly reduce toxicity with only a modest loss in potency, if any. This leads to an improvement in therapeutic index for a given target sequence. In certain cases, such improvements in therapeutic index are large enough to allow further drug development based on a compound targeted to a previously-toxic (but potent) sequence.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron.

### Certain Compounds Having Central Region Modifications

In certain embodiments, the oligomeric compounds herein comprise a gapmer comprising one or more an altered nucleotides in the central region of the gapmer. Each of such oligomeric compounds has a corresponding parent oligomeric compound that is identical to the first oligomeric compound except that is lacking the one or more altered nucleotides in the central region of the gapmer. Examples of such parent oligomeric compounds and their corresponding identical oligomeric compounds lacking the altered nucleotide are found in Tables 1 and 2 of Example 1 as well as throughout the Examples section. In Example 1, 558807 is the parent oligonucleotide. In certain embodiments, the central region of a parent oligomeric compound comprises only phosphodiester and/or phosphorothioate internucleoside linkages, unmodified nucleobases and/or 5-methylcytosine, and unmodified, 2′β-D-deoxyribosyl sugar moieties.

In certain embodiments, the present disclosure provides oligomeric compounds that comprise a gapmer comprising one or more altered nucleotides in the central region of the gapmer that have an increased therapeutic index an/or increased tolerability compared to the corresponding parent oligomeric compounds. In certain such embodiments, the modification or modifications of the central region of the oligomeric compounds with increased therapeutic index and/or increased tolerability are particularly useful in providing oligomeric compounds having reduced toxicity without significantly altering the potency. The modifications in the central region described herein can be at any position in the central region, and examples of embodiments comprising modifications at such positions are disclosed in the numbered embodiments and Examples. In certain embodiments, the altered nucleotide is an altered nucleoside attached to a phosphorothioate or phosphodiester internucleoside linkage. In a preferred embodiment, the altered nucleotide is at positions 1-4 of the central region of the modified oligonucleotide. In another preferred embodiment, the altered nucleotide comprises a nucleoside comprising a 2′-modified sugar moiety at position 2 of the central region of the modified oligonucleotide. In another preferred embodiment, the altered nucleotide comprises a nucleoside comprising a 5′-modified sugar moiety at positions 3 or 4 of the central region of the modified oligonucleotide. In another preferred embodiment, the altered nucleotide comprises a neutral internucleoside linkage between positions 2-3 or positions 3-4 of the central region of the modified oligonucleotide.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine nucleobase could be described as a DNA having an RNA sugar, or as an RNA having a DNA nucleobase.

Accordingly, nucleic acid sequences provided herein, including, but not limited to those in the sequence listing, are intended to encompass nucleic acids containing any combination of unmodified or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “AT^{m}CGAUCG,” wherein ^{m}C indicates a cytosine base comprising a methyl group at the 5-position. In certain cases, compounds comprising a uridine nucleobase and a 2′-OMe sugar moiety are compared to compounds comprising a thymidine nucoebase with a 2′-β-D-deoxyribosyl sugar moiety at the same position. While these compounds have different SEQ ID NO:, they are not considered distinct sequences, and they have identical target RNA. In certain cases, compounds comprising a cytosine nucleobase and a 2′-OMe sugar moiety are compared to compounds comprising a 5-methylcytosine nucleobase and a 2′-β-D-deoxyribosyl sugar moiety at the same position.

In the Examples below, modified oligonucleotides are represented by a chemistry notation, always shown in the 5′-to-3′ direction, of the format BₛₗBₛₗ^{m}Bₛ, where “B” or “^{m}B” represents the nucleobase, with a superscript “m” before “B” representing a 5-methyl modification, the subscript in postion “s” represents the sugar moiety, and the subscript in postion “l” represents the 5′-to-3′ internucleoside linkage.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1 Effect of Position-Specific 2′-Modifications on In Vitro Activity and In Vivo Liver Toxicity of Modified Oligonucleotides Complementary to CXCL12

Modified oligonucleotides were synthesized with kkk-x-d(9)-kkk, kkk-d-x-d(8)-kkk, kkk-dd-x-d(7)-kkk or kkk-d(3)-x-d(6)-kkk sugar motifs, respectively, where “x” represents a sugar moiety having the modification indicated in the table below, “k” represents a cEt, and “d” represents an unmodified, 2′-β-D-deoxyribosyl sugar moiety. These modified oligonucleotides were compared to the otherwise identical modified oligonucleotide lacking an altered nucleotide in the central region, a 3-10-3 cEt gapmer, having three cEt nucleosides in each of the 5′ and 3′ regions and 10 DNA nucleosides in the central region. Each internucleoside linkage is a phosphorothioate. The compounds in the table below are 100% complementary to mouse CXCL12, GENBANK NT_039353.7 truncated from 69430515 to 69445350 (SEQ ID NO: 1), at position 6877 to 6892.

TABLE 1
Modified oligonucleotides complementary to CXCL12

| | position | | | |
|---|---|---|---|---|
| | of altered | sugar | | |
| | nucleotide | modification | | SEQ |
| Compound | in central | of altered | | ID |
| ID | region | nucleotide | Chemistry Notation (5′-3′) | NO |
| 558807 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 936049 | 1 | 2′-OMe | Gₖₛ^{m}CₖₛAₖₛUₘₛG_{ds}T_{ds}T_{ds}^{m}C_{ds}T_{ds}^{m}C_{ds}A_{ds}^{m}C_{ds}A_{ds}TₖₛTₖₛAₖ | 19 |
| 828910 | 1 | 2′-MOE | Gₖₛ^{m}CₖₛAₖₛTₑₛG_{ds}T_{ds}T_{ds}^{m}C_{ds}T_{ds}^{m}C_{ds}A_{ds}^{m}C_{ds}A_{ds}TₖₛTₖₛAₖ | 18 |
| 936050 | 1 | cEt | Gₖₛ^{m}CₖₛAₖₛTₖₛG_{ds}T_{ds}T_{ds}^{m}C_{ds}T_{ds}^{m}C_{ds}A_{ds}^{m}C_{ds}A_{ds}TₖₛTₖₛAₖ | 18 |
| 936054 | 1 | 2′-FANA | Gₖₛ^{m}CₖₛAₖₛU_{(FANA)s}G_{ds}T_{ds}T_{ds}^{m}C_{ds}T_{ds}^{m}C_{ds}A_{ds}^{m}C_{ds}A_{ds}TₖₛTₖₛAₖ | 19 |
| 936048 | 1 | 2′-ribo-F | Gₖₛ^{m}CₖₛAₖₛU_{fs}G_{ds}T_{ds}T_{ds}^{m}C_{ds}T_{ds}^{m}C_{ds}A_{ds}^{m}C_{ds}A_{ds}TₖₛTₖₛAₖ | 19 |
| 936053 | 2 | 2′-OMe | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | 18 |
| 828911 | 2 | 2′-MOE | Gₖₛ^{m}CₖₛAₖₛT_{ds}GₑₛT_{ds}T_{ds}^{m}C_{ds}T_{ds}^{m}C_{ds}A_{ds}^{m}C_{ds}A_{ds}TₖₛTₖₛAₖ | 18 |
| 1070041 | 2 | cEt | Gₖₛ^{m}CₖₛAₖₛT_{ds}GₖₛT_{ds}T_{ds}^{m}C_{ds}T_{ds}^{m}C_{ds}A_{ds}^{m}C_{ds}A_{ds}TₖₛTₖₛAₖ | 18 |
| 1061314 | 2 | 2′-OH | Gₖₛ^{m}CₖₛAₖₛT_{ds}GᵣₛT_{ds}T_{ds}^{m}C_{ds}T_{ds}^{m}C_{ds}A_{ds}^{m}C_{ds}A_{ds}TₖₛTₖₛAₖ | 18 |
| | | (RNA) | | |
| 936051 | 2 | 2′-FANA | Gₖₛ^{m}CₖₛAₖₛT_{ds}G_{(FANA)s}T_{ds}T_{ds}mC_{ds}T_{ds}mC_{ds}A_{ds}mC_{ds}A_{ds}TₖₛTₖₛAₖ | 18 |
| 936052 | 2 | 2′-ribo-F | Gₖₛ^{m}CₖₛAₖₛT_{ds}G_{fs}T_{ds}T_{ds}^{m}C_{ds}T_{ds}^{m}C_{ds}A_{ds}^{m}C_{ds}A_{ds}TₖₛTₖₛAₖ | 18 |
| 892826 | 3 | 2′-OMe | Gₖₛ^{m}CₖₛAₖₛT_{ds}G_{ds}UₘₛT_{ds}^{m}C_{ds}T_{ds}^{m}C_{ds}A_{ds}^{m}C_{ds}A_{ds}TₖₛTₖₛAₖ | 20 |
| 828912 | 3 | 2′-MOE | Gₖₛ^{m}CₖₛAₖₛT_{ds}G_{ds}Te_{S}T_{ds}^{m}C_{ds}T_{ds}^{m}C_{ds}A_{ds}^{m}C_{ds}A_{ds}TₖₛTₖₛAₖ | 18 |
| 892816 | 3 | cEt | Gₖₛ^{m}CₖₛAₖₛT_{ds}G_{ds}TₖₛT_{ds}^{m}C_{ds}T_{ds}^{m}C_{ds}A_{ds}^{m}C_{ds}A_{ds}TₖₛTₖₛAₖ | 18 |
| 895596 | 3 | 2′-FANA | Gₖₛ^{m}CₖₛAₖₛT_{ds}G_{ds}U_{(FANA)s}T_{ds}^{m}C_{ds}T_{ds}^{m}C_{ds}A_{ds}^{m}C_{ds}A_{ds}TₖₛTₖₛAₖ | 20 |
| 892821 | 3 | 2′-ribo-F | Gₖₛ^{m}CₖₛAₖₛT_{ds}G_{ds}U_{fS}T_{ds}^{m}C_{ds}T_{ds}^{m}C_{ds}A_{ds}^{m}C_{ds}A_{ds}TₖₛTₖₛAₖ | 20 |
| 892819 | 4 | 2′-OMe | Gₖₛ^{m}CₖₛAₖₛT_{ds}G_{ds}T_{ds}Uₘₛ^{m}C_{ds}T_{ds}^{m}C_{ds}A_{ds}^{m}C_{ds}A_{ds}TₖₛTₖₛAₖ | 21 |
| 828913 | 4 | 2′-MOE | Gₖₛ^{m}CₖₛAₖₛT_{ds}G_{ds}T_{ds}Tₑₛ^{m}C_{ds}T_{ds}^{m}C_{ds}A_{ds}^{m}C_{ds}A_{ds}TₖₛTₖₛAₖ | 18 |
| 892817 | 4 | cEt | Gₖₛ^{m}CₖₛAₖₛT_{ds}G_{ds}T_{ds}Tₖₛ^{m}C_{ds}T_{ds}^{m}C_{ds}A_{ds}^{m}C_{ds}A_{ds}TₖₛTₖₛAₖ | 18 |
| 895595 | 4 | 2′-FANA | Gₖₛ^{m}CₖₛAₖₛT_{ds}G_{ds}T_{ds}U_{(FANA)s}^{m}C_{ds}T_{ds}^{m}C_{ds}A_{ds}^{m}C_{ds}A_{ds}TₖₛTₖₛAₖ | 21 |
| 892822 | 4 | 2′-ribo-F | Gₖₛ^{m}CₖₛAₖₛT_{ds}G_{ds}T_{ds}U_{fs}^{m}C_{ds}T_{ds}^{m}C_{ds}A_{ds}^{m}C_{ds}TₖₛTₖₛAₖ | 21 |

A subscript “d” indicates an unmodified, 2′-β-D-deoxyribosyl sugar moiety.
A subscript “e” indicates a 2′-MOE-β-D-ribofuranosyl sugar moiety.
A subscript “m” indicates a 2′-O-methyl-β-D-ribofuranosyl sugar moiety.
A subscript “k” indicates a cEt.
A subscript “s” indicates a phosphorothioate internucleoside linkage.
A subscript “f” indicates a 2′-F-β-D-2′-deoxyribosyl sugar moiety.
A subscript “(FANA)” indicates a 2′-F-β-D-arabinofuranosyl modified sugar moiety.
A superscript “m” before a C indicates 5-methyl Cytosine.

For in vitro activity studies, approximately 20,000 mouse 3T3-L1 cells were electroporated with 0, 27 nM, 82 nM, 247 nM, 740 nM, 2,220 nM, 6,660 nM or 20,000 nM antisense oligonucleotide. mRNA was harvested and analyzed by RT-qPCR. CXCL12 mRNA was detected with primer probe set RTS2605 (forward sequence CCAGAGCCAACGTCAAGCAT, SEQ ID NO: 9; reverse sequence: CAGCCGTGCAACAATCTGAA, SEQ ID NO: 10; probe sequence: TGAAAATCCTCAACACTCCAAACTGTGCC, SEQ ID NO: 11) and RAPTOR mRNA was detected with primer probe set RTS3420 (forward sequence GCCCTCAGAAAGCTCTGGAA, SEQ ID NO: 12; reverse sequence: TAGGGTCGAGGCTCTGCTTGT, SEQ ID NO: 13; probe sequence: CCATCGGTGCAAACCTACAGAAGCAGTATG, SEQ ID NO: 14). RAPTOR is a sentinel gene that can be indicative of toxicity, as described in US 20160160280, hereby incorporated by reference.

For acute in vivo toxicity studies, three BALB/C mice per group were administered 150 mg/kg modified oligonucleotide by subcutaneous injection and sacrificed 72 hours later. Three mice were administered an injection of saline as a control. Plasma levels of ALT were measured using an automated clinical chemistry analyzer. Increased ALT is indicative of acute liver toxicity.

TABLE 2
Activity and toxicity of modified oligonucleotides
complementary CXCL12

| | position | | | | |
|---|---|---|---|---|---|
| | of altered | sugar | in vitro | in vitro | ALT @ |
| | nucleotide | modification | CXCL12 | RAPTOR | 150 |
| Compound | in central | of altered | IC₅₀ | IC₅₀ | mg/kg |
| ID | region | nucleotide | (μM) | (μM) | (IU/L) |
| PBS | n/a | n/a | n/a | n/a | 23 |
| 558807 | n/a | n/a | 0.2 | 1.26 | n.d.** |
| 936049 | 1 | 2′-OMe | 0.17 | 4.8 | 8622 |
| 828910 | 1 | 2′-MOE | 0.12 | 7.2 | 2175 |
| 936050 | 1 | cEt | 0.15 | 6.5 | 912 |
| 936054 | 1 | 2′-FANA | 0.12 | 9.9 | 5755 |
| 936048 | 1 | 2′-ribo-F | 0.15 | 1.9 | death |
| 936053 | 2 | 2′-OMe | 0.17 | >>10 | 46 |
| 828911 | 2 | 2′-MOE | 0.42 | >>10 | 27 |
| 1070041 | 2 | cEt | 0.52 | n.d. | 96 |
| 1061314 | 2 | 2′-OH | n.d. | n.d. | 26 |
| | | (RNA) | | | |
| 936051 | 2 | 2′-FANA | 0.12 | 2.34 | death |
| 936052 | 2 | 2′-ribo-F | 0.19 | 13.5 | 1110 |
| 892826 | 3 | 2′-OMe | 0.21 | 7.1 | 10463 |
| 828912 | 3 | 2′-MOE | 0.28 | 10 | 701 |
| 892816 | 3 | cEt | 0.17 | 11 | 278 |
| 895596 | 3 | 2′-FANA | 0.12 | 7.4 | 17369 |
| 892821 | 3 | 2′-ribo-F | 0.18 | 4.5 | 6333 |
| 892819 | 4 | 2′-OMe | 0.18 | >10 | 565 |
| 828913 | 4 | 2′-MOE | 0.22 | 10 | 2474 |
| 892817 | 4 | cEt | 0.23 | 9 | 5264 |
| 895595 | 4 | 2′-FANA | 0.08 | 8.8 | 22082 |
| 892822 | 4 | 2′-ribo-F | 0.04 | 4.85 | 4020 |

**558807 treatment at 16.7 mg/kg leads to an ALT of 586 IU/L in this experiment; mice that are treated with 558807 at 150 mg/kg typically experience death before 72 hours post-treatment.

For the in vitro study reported in the tables below, 3T3-L1 cells were electroporated with 27 nM, 80 nM, 250 nM, 740 nM, 2, 222 nM, 6,667 nM, or 20,000 nM of modified oligonucleotide and levels of P21 and Gadd45a were measured by RT-qPCR. Levels of Gadd45a were analyzed using primer probe set Mm00432802_m1 (ThermoFisher). Levels of P21 were analyzed using primer probe set Mm04207341_m1 (ThermoFisher).

TABLE 2b
in vitro P21 Expression in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 558807 | 110 | 98 | 69 | 99 | 117 | 228 | 488 |
| 936049 | 63 | 116 | 96 | 93 | 121 | 151 | 199 |
| 936053 | 151 | 144 | 158 | 160 | 152 | 143 | 155 |
| 892826 | 140 | 104 | 104 | 128 | 138 | 181 | 177 |

TABLE 2c
in vitro Gadd45a Expression in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 558807 | 115 | 120 | 111 | 136 | 155 | 237 | 298 |
| 936049 | 101 | 124 | 131 | 158 | 172 | 212 | 276 |
| 936053 | 144 | 227 | 175 | 203 | 197 | 201 | 193 |
| 892826 | 132 | 114 | 134 | 152 | 147 | 163 | 158 |

For the in vitro study reported in the tables below, b.END cells were electroporated with 27 nM, 80 nM, 250 nM, 740 nM, 2, 222 nM, 6,667 nM, or 20,000 nM of modified oligonucleotide and levels of P21 were measured by RT-qPCR using primer probe set Mm04207341_m1 (ThermoFisher).

TABLE 2d
in vitro P21 Expression in b.END cells

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 558807 | 138 | 117 | 93 | 73 | 107 | 160 | 226 |
| 936053 | 108 | 112 | 96 | 90 | 111 | 101 | 118 |
| 892826 | 112 | 112 | 114 | 107 | 117 | 116 | 137 |
| 1061314 | 126 | 128 | 103 | 115 | 128 | 122 | 112 |
| 936051 | 114 | 113 | 109 | 118 | 117 | 123 | 178 |
| 936052 | 109 | 116 | 102 | 100 | 112 | 119 | 138 |
| 828911 | 115 | 108 | 120 | 113 | 114 | 115 | 122 |
| 1070041 | 101 | 100 | 109 | 104 | 104 | 120 | 132 |

TABLE 2e
in vitro Gadd45a Expression in b.END cells

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 558807 | 558807 | 86 | 132 | 93 | 101 | 178 | 220 |
| 936053 | 936053 | 111 | n.d. | 148 | 150 | 200 | 215 |
| 892826 | 892826 | 134 | 177 | 160 | 170 | 177 | 162 |
| 1061314 | 1061314 | 135 | 149 | 150 | 197 | 199 | 184 |
| 936051 | 136 | 132 | 152 | 185 | 199 | 193 | 258 |
| 936052 | 125 | 160 | 146 | 173 | 210 | 201 | 228 |
| 828911 | 121 | 154 | 158 | 193 | 190 | 189 | 249 |
| 1070041 | 118 | 139 | 163 | 194 | 225 | 301 | 313 |

For the in vivo activity and toxicity study in the table below, 2 or 3 BALB/C mice per group were administered modified oligonucleotide at 1.8 mg/kg, 5.5 mg/kg, or 16.7 mg/kg by subcutaneous injection and sacrificed after 72 hours.

TABLE 2f
in vivo Activity and Toxicity

| | position | | | | |
|---|---|---|---|---|---|
| | of altered | sugar | in vivo | ALT | |
| Com- | nucleotide | modification | CXCL12 | @5.5 | ALT @ |
| pound | in central | of altered | ED50 | mg/kg | 16.7 mg/kg |
| ID | region | nucleotide | (mg/kg) | (IU/L) | (IU/L) |
| PBS | n/a | n/a | n/a | 30 (@0 mg/kg) | |
| 558807 | n/a | n/a | 1.6 | 40 | 1721 |
| 936051 | 2 | 2′-FANA | 0.15 | 44 | 4285 |
| 936053 | 2 | 2′-OMe | 5.5 | 27 | 25 |
| 828911 | 2 | 2′-MOE | 14 | 36 | 25 |
| 936052 | 2 | 2′-ribo-F | 2.9 | 26 | 29 |

For in vivo activity and toxicity study in the table below, 3 BALB/C mice per group were administered modified oligonucleotide by subcutaneous injection and sacrificed after 72 hours. Compound 558807 was dosed at 1.8, 5.5, or 16.7 mg/kg, compounds 828911, 936052 and 936053 were dosed at 1.8, 5.5, 16.7 and 50 mg/kg, and compounds 1061315 and 1070041 were dosed at 1.8, 5.5, 16.7, 50 or 150 mg/kg. Tissue were collected and mRNA was isolated and levels of CXCL12 were measured by RT-qPCR with primer probe set RTS2605 as described above. Levels of Gadd45a were analyzed using primer probe set Mm00432802_m1 (ThermoFisher). Levels of P21 were analyzed using primer probe set Mm04207341_m1 (ThermoFisher). Levels of Tnfrsf10b were analyzed using primer probe set Mm00457866_m1 (ThermoFisher). Expression levels were normalized with Ribogreen® and are presented relative to levels in mice treated with PBS.

TABLE 2g
In Vivo Activity and Toxicity of modified
oligonucleotides complementary to CXCL12

| | position | sugar | | | |
|---|---|---|---|---|---|
| | of altered | modifica- | in vivo | | |
| Com- | nucleotide | tion of | CXCL12 | ALT @50 | ALT @ |
| pound | in central | altered | ED50 | mg/kg | 150 mg/kg |
| ID | region | nucleotide | (mg/kg) | (IU/L) | (IU/L) |
| PBS | n/a | n/a | n/a | 28 (@0 mg/kg) | |
| 558807 | n/a | n/a | 2.7 | n.d.** | |
| 936053 | 2 | 2′-OMe | 4.9 | 23 | n.d. |
| 828911 | 2 | 2′-MOE | 14 | 27 | n.d. |
| 1070041 | 2 | cEt | 29 | 25 | 78 |
| 1061314 | 2 | 2′-OH | 78 | 21 | 24 |
| | | (RNA) | | | |
| 936052 | 2 | 2′-ribo-F | 4.2 | 39 | n.d. |

**558807 treatment at 16.7 mg/kg leads to an ALT of 586 IU/L; mice that are treated with 558807 at 150 mg/kg typically experience death before 72 hours post-treatment.

TABLE 2h
In Vivo Dose-response of Gadd45a mRNA upon treatment with
modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of Gadd45a mRNA (% Control) | | | | |
| 558807 | 100 | 172 | 856 | n/a | n/a |
| 936053 | 61 | 99 | 91 | 92 | n/a |
| 828911 | 80 | 100 | 96 | 100 | n/a |
| 1070041 | 128 | 225 | 139 | 177 | 169 |
| 1061314 | 112 | 84 | 89 | 105 | 180 |
| 936052 | 84 | 80 | 134 | 126 | n/a |

TABLE 2i
In Vivo Dose-response of Tnfrsf10b mRNA upon treatment
with modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of Tnfrsf10b mRNA (% Control) | | | | |
| 558807 | 102 | 267 | 790 | n/a | n/a |
| 936053 | 106 | 111 | 130 | 100 | n/a |
| 828911 | 120 | 116 | 95 | 102 | n/a |
| 1070041 | 106 | 139 | 252 | 483 | 1021 |
| 1061314 | 79 | 66 | 81 | 136 | 220 |
| 936052 | 82 | 101 | 183 | 138 | n/a |

TABLE 2j
In Vivo Dose-response of P21 mRNA upon treatment with
modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of P21 mRNA (% Control) | | | | |
| 558807 | 100 | 61 | 609 | n/a | n/a |
| 936053 | 104 | 94 | 104 | 89 | n/a |
| 828911 | 90 | 145 | 52 | 92 | n/a |
| 1070041 | 28 | 93 | 83 | 132 | 264 |
| 1061314 | 45 | 59 | 30 | 34 | 178 |
| 936052 | 70 | 71 | 51 | 101 | n/a |

For the in vivo activity study in the tables below, 3 BALB/C mice per group were administered 3.37, 11, 33, or 100 mg/kg doses of modified oligonucleotide, as indicated in the table below, by subcutaneous injection and sacrificed 72 hours later. Liver mRNA was isolated an analyzed by RT-PCR as described in above. Expression levels were normalized with Ribogreen® and are expressed relative to PBS-treated control mice.

TABLE 2k
Activity and toxicity of modified
oligonucleotides complementary to CXCL12

| | position | | | |
|---|---|---|---|---|
| | of altered | sugar | | |
| | nucleotide | modification | | |
| Compound | in central | of altered | ALT (IU/L) | ALT (IU/L) |
| ID | region | nucleotide | at 100 mg/kg | at 33 mg/kg |
| 558807 | n/a | n/a | death | 3740 |
| 936049 | 1 | 2′-OMe | 3060 | 612 |
| 936053 | 2 | 2′-OMe | 42 | 21 |
| 892826 | 3 | 2′-OMe | 1127 | 2281 |

TABLE 2l
In Vivo Dose-response of CXCL12 mRNA upon treatment with
modified oligonucleotides complementary to CXCL12

| Compound | 3.37 mg/kg | 11 mg/kg | 33 mg/kg | 100 mg/kg |
|---|---|---|---|---|
| ID | Expression level of CXCL12 mRNA (% Control) | | | |
| 558807 | 95 | 29 | 12 | n.d. |
| 936049 | 102 | 50 | 22 | 14 |
| 936053 | 100 | 70 | 40 | 31 |
| 892826 | 100 | 49 | 16 | 10 |

TABLE 2m
In Vivo Dose-response of P21 mRNA upon treatment with
modified oligonucleotides complementary to CXCL12

| Compound | 3.37 mg/kg | 11 mg/kg | 33 mg/kg | 100 mg/kg |
|---|---|---|---|---|
| ID | Expression level of P21 mRNA (% Control) | | | |
| 558807 | 194 | 186 | 32700 | n.d. |
| 936049 | 126 | 127 | 3156 | 21746 |
| 936053 | 100 | 49 | 89 | 185 |
| 892826 | 60 | 60 | 2401 | 12981 |

### Example 2 Effect of Position-Specific 5′-Modifications on In Vitro Activity and In Vivo Liver Toxicity of Modified Oligonucleotides Complementary CXCL12

Modified oligonucleotides containing 5′-methyl, 5′-allyl, and 5′-ethyl modifications at various positions were synthesized. Procedures for the synthesis of 5′-methyl and 5′-allyl analogs are detailed in WO2013022967. Procedures for the synthesis of 5′-ethyl analogs are detailed herein below in Example 39. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleobases in the central region. The compounds in the table below are 100% complementary to mouse CXCL12, GENBANK NT_039353.7 truncated from 69430515 to 69445350 (SEQ ID NO: 1), at position 6877 to 6892.

TABLE 3
Modified oligonucleotides complementary to CXCL12

| | position | | | |
|---|---|---|---|---|
| | of | | | |
| | altered | sugar | | |
| | nucleotide | modification | | |
| | in | of | | SEQ |
| Compound | central | altered | | ID |
| ID | region | nucleotide | Chemistry Notation (5′-3′) | NO |
| 558807 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 1123320 | 2 | 5′-(S)- | Gₖₛ^{m}CₖₛAₖₛT_{ds}G_{[(S)-μ]s}T_{ds}T_{ds}^{m}C_{ds}T_{ds}^{m}C_{ds}A_{ds}^{m}C_{ds}A_{ds}TₖₛTₖₛAₖ | 18 |
| | | Me | | |
| 1123322 | 2 | 5′-(R)- | Gₖₛ^{m}CₖₛAₖₛT_{ds}G_{[(R)-μ]s}T_{ds}T_{ds}^{m}C_{ds}T_{ds}^{m}C_{ds}A_{ds}^{m}C_{ds}A_{ds}TₖₛTₖₛAₖ | 18 |
| | | Me | | |
| 1123479 | 2 | 5′-(R,S)- | Gₖₛ^{m}CₖₛAₖₛT_{ds}G_{[γ]s}T_{ds}T_{ds}^{m}C_{ds}T_{ds}^{m}C_{ds}A_{ds}^{m}C_{ds}A_{ds}TₖₛTₖₛAₖ | 18 |
| | | allyl | | |
| 942943 | 3 | 5′-(R)- | Gₖₛ^{m}CₖₛAₖₛT_{ds}G_{ds}T_{[(R)-μ]s}T_{ds}^{m}C_{ds}T_{ds}^{m}C_{ds}A_{ds}^{m}C_{ds}A_{ds}TₖₛTₖₛAₖ | 18 |
| | | Me | | |
| 957908 | 3 | 5′-(S)- | Gₖₛ^{m}CₖₛAₖₛT_{ds}G_{ds}T_{[(S)-μ]s}T_{ds}^{m}C_{ds}T_{ds}^{m}C_{ds}A_{ds}^{m}C_{ds}A_{ds}TₖₛTₖₛAₖ | 18 |
| | | Me | | |
| 957910 | 3 | 5′-(R)- | Gₖₛ^{m}CₖₛAₖₛT_{ds}G_{ds}T_{[(R)-γ]s}T_{ds}^{m}C_{ds}T_{ds}^{m}C_{ds}A_{ds}^{m}C_{ds}A_{ds}TₖₛTₖₛAₖ | 18 |
| | | allyl | | |
| 957912 | 3 | 5′-(S)- | Gₖₛ^{m}CₖₛAₖₛT_{ds}G_{ds}T_{[(S)-γ]s}T_{ds}^{m}C_{ds}T_{ds}^{m}C_{ds}A_{ds}^{m}C_{ds}A_{ds}TₖₛTₖₛAₖ | 18 |
| | | allyl | | |
| 1175787 | 3 | 5′-(R)-Et | Gₖₛ^{m}CₖₛAₖₛT_{ds}G_{ds}T_{[(R)-ε]s}T_{ds}^{m}C_{ds}T_{ds}^{m}C_{ds}A_{ds}^{m}C_{ds}A_{ds}TₖₛTₖₛAₖ | 18 |
| 1175785 | 3 | 5′-(S)-Et | Gₖₛ^{m}CₖₛAₖₛT_{ds}G_{ds}T_{[(S)-ε]s}T_{ds}^{m}C_{ds}T_{ds}^{m}C_{ds}A_{ds}^{m}C_{ds}A_{ds}TₖₛTₖₛAₖ | 18 |
| 942944 | 4 | 5′-(R)- | Gₖₛ^{m}CₖₛAₖₛT_{ds}G_{ds}T_{ds}T_{[(R)-μ]s}^{m}C_{ds}T_{ds}^{m}C_{ds}A_{ds}^{m}C_{ds}A_{ds}TₖₛTₖₛAₖ | 18 |
| | | Me | | |
| 957909 | 4 | 5′-(S)- | Gₖₛ^{m}CₖₛAₖₛT_{ds}G_{ds}T_{ds}T_{[(S)-μ]s}^{m}C_{ds}T_{ds}^{m}C_{ds}A_{ds}^{m}C_{ds}A_{ds}TₖₛTₖₛAₖ | 18 |
| | | Me | | |
| 957911 | 4 | 5′-(R)- | Gₖₛ^{m}CₖₛAₖₛT_{ds}G_{ds}T_{ds}T_{[(R)-γ]S}^{m}C_{ds}T_{ds}^{m}C_{ds}A_{ds}^{m}C_{ds}A_{ds}TₖₛTₖₛAₖ | 18 |
| | | allyl | | |
| 957913 | 4 | 5′-(S)- | Gₖₛ^{m}CₖₛAₖₛT_{ds}G_{ds}T_{ds}T_{[(S)-γ]s}^{m}C_{ds}T_{ds}^{m}C_{ds}A_{ds}^{m}C_{ds}A_{ds}TₖₛTₖₛAₖ | 18 |
| | | allyl | | |
| 1175786 | 4 | 5′-(R)-Et | Gₖₛ^{m}CₖₛAₖₛT_{ds}G_{ds}T_{ds}T_{[(R)-ε]s}^{m}C_{ds}T_{ds}^{m}C_{ds}A_{ds}^{m}C_{ds}A_{ds}TₖₛTₖₛAₖ | 18 |
| 1175782 | 4 | 5′-(S)-Et | Gₖₛ^{m}CₖₛAₖₛT_{ds}G_{ds}T_{ds}T_{[(S)-ε]s}^{m}C_{ds}T_{ds}^{m}C_{ds}A_{ds}^{m}C_{ds}A_{ds}TₖₛTₖₛAₖ | 18 |

A subscript “d” indicates an unmodified, 2′-β-D-deoxyribosyl sugar moiety.
A subscript “k” indicates a cEt.
A subscript “s” indicates a phosphorothioate internucleoside linkage.
A subscript “[μ]″ indicates a 5′-(R,S)-Me-β-D-2’-deoxyribosyl sugar moiety.
A subscript “[(R)-μ]″ indicates a 5’-(R)-Me-β-D-2′-deoxyribosyl sugar moiety.
A subscript “[(S)-μ]” indicates a 5′-(S)-Me-β-D-2′-deoxyribosyl sugar moiety.
A subscript “[γ]” indicates a 5′-(R,S)-allyl-β-D-2′-deoxyribosyl sugar moiety.
A subscript “[(R)-γ]″ indicates a 5′-(R)-allyl-β-D-2′-deoxyribosyl sugar moiety.
A subscript “[(S)-γ]” indicates a 5′-(S)-allyl-β-D-2′-deoxyribosyl sugar moiety.
A subscript “[ε]” indicates a 5′-(R,S)-ethyl-β-D-2′-deoxyribosyl sugar moiety.
A subscript “[(R)-ε]” indicates a 5′-(R)-ethyl-β-D-2′-deoxyribosyl sugar moiety.
A subscript “[(S)-ε]” indicates a 5′-(S)-ethyl-β-D-2′-deoxyribosyl sugar moiety.

### Experimental Procedures & Results

In vitro activity and in vivo activity and toxicity experiments were performed essentially as described in Example 1. For in vivo toxicity studies, a single BALB/C mouse per modified oligonucleotide was administered 150 mg/kg modified oligonucleotide by subcutaneous injection and sacrificed 72 hours later. For the in vivo activity study in the table below, 2 BALB/C mice per group were administered 1.8 mg/kg, 5.5 mg/kg, 16.7 mg/kg, 50 mg/kg dose of modified oligonucleotide subcutaneously and sacrificed after 72 hours. For 558807, only 1.8 mg/kg, 5.5 mg/kg, and 16.7 mg/kg doses were tested for dose response, due to acute toxicity of higher doses. Tissues were collected and liver mRNA was isolated and levels of CXCL12 were measured by RT-qPCR with primer probe set RTS2605 as described above.

TABLE 4
Activity and toxicity of modified oligonucleotides complementary CXCL12

| | position | | | | | |
|---|---|---|---|---|---|---|
| | of altered | sugar | In vitro | Raptor | | |
| | nucleotide | modification | IC50 | IC50 | in vivo | ALT |
| Compound | in central | of altered | CXCL12 | (tox | ED50 | (at 150 |
| ID | region | nucleotide | (μM) | marker) | (mg/kg) | mg/kg) |
| 558807 | n/a | Parent | 0.11 | 1.3 | 2.9 | n.d.** |
| 942943 | 3 | 5′-(R)-Me | 0.118 | 23 | 2.8 | 2466 |
| 942944 | 4 | 5′-(R)-Me | 0.169 | 22 | 3 | 233 |
| 957908 | 3 | 5′-(S)-Me | 0.193 | 33 | 3.7 | 52 |
| 957909 | 4 | 5′-(S)-Me | 0.159 | 4 | 2.2 | 1267 |
| 957910 | 3 | 5′-(R)-allyl | 0.239 | >>20 | 3.6 | 32 |
| 957911 | 4 | 5′-(R)-allyl | 0.269 | >>20 | 6.4 | 30 |
| 957912 | 3 | 5′-(S)-allyl | 0.234 | >>20 | 5.1 | 30 |
| 957913 | 4 | 5′-(S)-allyl | 0.263 | >>20 | 5.7 | 32 |

**Not tested in this experiment; mice that are treated with 558807 at 150 mg/kg typically experience death before 72 hours post-treatment.

For the in vivo activity and toxicity study reported in the table below, 2 BALB/C mice per group were administered 5 mg/kg or 150 mg/kg modified oligonucleotide subcutaneously and sacrificed 72 hours later. Plasma levels of ALT were measured and liver mRNA was analyzed for target reduction as in example 1 above.

TABLE 5
Activity and toxicity of modified oligonucleotides complementary CXCL12

| | position | | | | | |
|---|---|---|---|---|---|---|
| | of altered | sugar | CXCL12 | CXCL12 | | |
| | nucleotide | modification | mRNA | mRNA | ALT | ALT |
| Compound | in central | of altered | (% control) | (% control) | 5 mg/kg | 150 mg/kg |
| ID | region | nucleotide | 5 mg/kg | 150 mg/kg | (IU/L) | (IU/L) |
| 558807 | n/a | n/a | n.d. | n.d. | n.d. | n.d.** |
| 942943 | 3 | 5′-(R)-Me | 28 | 3 | 27 | 4407 |
| 957910 | 3 | 5′-(R)-allyl | 53 | 7 | 24 | 38 |
| 1175787 | 3 | 5′-(R)-Et | 57 | 6 | 27 | 39 |
| 1175785 | 3 | 5′-(S)-Et | 46 | 8 | 25 | 45 |
| 957909 | 4 | 5′-(S)-Me | 30 | 7 | 22 | 7133 |
| 957913 | 4 | 5′-(S)-allyl | 59 | 10 | 30 | 37 |
| 1175786 | 4 | 5′-(R)-Et | 44 | 35 | 24 | 44 |
| 1175782 | 4 | 5′-(S)-Et | 52 | 7 | 26 | 131 |

**Not tested in this experiment; mice that are treated with 558807 at 150 mg/kg typically experience death before 72 hours post-treatment.

For the results in the tables below, in vivo activity and toxicity experiments were performed essentially as described in Example 1. For in vivo toxicity studies, two BALB/C mice per group was administered 50 or 150 mg/kg modified oligonucleotide by subcutaneous injection and sacrificed 72 hours later. One animal was administered an injection of saline as a control. Plasma levels of ALT were measured using an automated clinical chemistry analyzer.

For the in vivo activity study in the tables below, 2 BALB/C mice per group were administered 1.8 mg/kg, 5.5 mg/kg, 16.7 mg/kg, 50 mg/kg, or 150 mg/kg doses of modified oligonucleotide, as indicated in the table below, by subcutaneous injection and sacrificed 72 hours later. For 558807, only 1.8 mg/kg, 5.5 mg/kg, and 16.7 mg/kg doses were tested for dose response, due to acute toxicity of higher doses. Liver mRNA was isolated an analyzed by RT-PCR as described in Example 1 above. Expression levels were normalized with Ribogreen® and are expressed relative to PBS-treated control mice.

TABLE 6
Activity and toxicity of modified oligonucleotides
complementary CXCL12

| | position | | | | |
|---|---|---|---|---|---|
| | of altered | sugar | | | |
| | nucleotide | modification | in vivo | ALT | ALT |
| Compound | in central | of altered | ED₅₀ | (IU/L) at | (IU/L) at |
| ID | region | nucleotide | (mg/kg) | 150 mg/kg | 50 mg/kg |
| 558807 | n/a | n/a | 1.7* | n.d.** | n.d.** |
| 1123320 | 2 | 5′-(R)-Me | 2.8 | 7448 | 3987 |
| 1123322 | 2 | 5′-(S)-Me | 2.1 | 5181 | 1912 |
| 1123479 | 2 | 5′-(R,S)-allyl | 6.1 | 2562 | 56 |

*Compound 558807 was only dosed at 1.8, 5.5, and 16.7 mg/kg
**Not tested in this experiment; mice that are treated with 558807 150 mg/kg typically experience death within 72 hours post-treatment.

TABLE 6b
In Vivo Dose-response of Gadd45a mRNA upon treatment with
modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of Gadd45a mRNA (% Control) | | | | |
| 558807 | 83 | 143 | 188 | n/a | n/a |
| 1123320 | 68 | 80 | 114 | 387 | 683 |
| 1123322 | 105 | 61 | 169 | 141 | 575 |
| 1123479 | 88 | 70 | 75 | 273 | 141 |

TABLE 6c
In Vivo Dose-response of Tnfrsf10b mRNA upon treatment
with modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of Tnfrsf10b mRNA (% Control) | | | | |
| 558807 | 115 | 119 | 195 | n/a | n/a |
| 1123320 | 78 | 115 | 77 | 1,802 | 6,928 |
| 1123322 | 95 | 75 | 231 | 1,036 | 8,281 |
| 1123479 | 174 | 132 | 125 | 303 | 1,423 |

TABLE 6d
In Vivo Dose-response of P21 mRNA upon treatment with
modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of P21 mRNA (% Control) | | | | |
| 558807 | 144 | 123 | 1212 | n/a | n/a |
| 1123320 | 109 | 224 | 114 | 17,332 | 51,431 |
| 1123322 | 218 | 92 | 303 | 10,383 | 75,226 |
| 1123479 | 271 | 209 | 295 | 838 | 12,248 |

TABLE 7
Activity and toxicity of modified oligonucleotides
complementary CXCL12

| | position | | | | |
|---|---|---|---|---|---|
| | of altered | sugar | | | |
| | nucleotide | modification | in vivo | ALT | ALT |
| Compound | in central | of altered | EC₅₀ | (IU/L) at | (IU/L) at |
| ID | region | nucleotide | (mg/kg) | 150 mg/kg | 50 mg/kg |
| 936053 | 2 | 2′-OMe | 4.9 | 49 | 23 |
| 1175782 | 4 | 5′-(S)-Et | 3.7 | 153 | 37 |
| 1175785 | 3 | 5′-(S)-Et | 6.6 | 34 | 24 |
| 1175786 | 4 | 5′-(R)-Et | 3.5 | 33 | 26 |
| 1175787 | 3 | 5′-(R)-Et | 5.8 | 39 | 28 |

### Example 3 Effect of Position-Specific 2′-Modifications on In Vitro Activity and In Vivo Liver Toxicity of Modified Oligonucleotides Complementary to Factor XI

Modified oligonucleotides were synthesized as indicated in the table below, comprising an altered nucleotide at positions 1-3 of the central region. These modified oligonucleotides were compared to the otherwise identical modified oligonucleotide lacking an altered nucleotide in the central region, a 3-10-3 cEt gapmer, containing three cEt nucleosides in each of the 5′ and 3′ regions and 10 unmodified DNA nucleosides in the central region. Each internucleoside linkage is a phosphorothioate internucleoside linkage. The compounds in the table below are 100% complementary to the complement of mouse Factor XI, GENBANK NT_039460.6 truncated from 6086000 to 6111000 (SEQ ID NO: 2), at position 11699 to 11714.

TABLE 8
Modified oligonucleotides complementary to Factor XI

| | position of | | | |
|---|---|---|---|---|
| | altered | sugar | | |
| | nucleotide in | modification | | SEQ |
| Compound | central | of altered | | ID |
| ID | region | nucleotide | Chemistry Notation (5′-3′) | NO |
| 464917 | n/a | n/a | GₖₛTₖₛ^{m}CₖₛT_{ds}G_{ds}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 982033 | 1 | 2′-OMe | GₖₛTₖₛ^{m}CₖₛUₘₛG_{ds}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 143 |
| 982034 | 2 | 2′-OMe | GₖₛTₖₛ^{m}CₖₛT_{ds}GₘₛT_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 985292 | 2 | 2′-MOE | GₖₛTₖₛ^{m}CₖₛT_{ds}GₑₛT_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 985293 | 2 | cEt | GₖₛTₖₛ^{m}CₖₛT_{ds}GₖₛT_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1011274 | 2 | 2′-FANA | GₖₛTₖₛ^{m}CₖₛT_{ds}G_{(FANA)s}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 604581 | 2 | 2′-ribo-F | GₖₛTₖₛ^{m}CₖₛT_{ds}G_{fs}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 982035 | 3 | 2′-OMe | GₖₛTₖₛ^{m}CₖₛT_{ds}G_{ds}UₘₛG_{ds}^{m}C_{ds}A_{ds}T_{ds}^{m}C_{ds}T_{ds}^{m}C_{ds}Tₖₛ^{m}Cₖₛ^{m}Cₖ | 23 |
| 985294 | 3 | 2′-MOE | GₖₛTₖₛ^{m}CₖₛT_{ds}G_{ds}TₑₛG_{ds}^{m}C_{ds}A_{ds}T_{ds}^{m}C_{ds}T_{ds}^{m}C_{ds}Tₖₛ^{m}Cₖₛ^{m}Cₖ | 22 |
| 985295 | 3 | cEt | GₖₛTₖₛ^{m}CₖₛT_{ds}G_{ds}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1011276 | 3 | 2′-FANA | GₖₛTₖₛ^{m}CₖₛT_{ds}G_{ds}U_{(FANA)s}G_{ds}^{m}C_{ds}A_{ds}T_{ds}^{m}C_{ds}T_{ds}^{m}C_{ds}Tₖₛ^{m}Cₖₛ^{m}Cₖ | 23 |
| 605933 | 3 | 2′-ribo-F | GₖₛTₖₛ^{m}CₖₛT_{ds}G_{ds}U_{fs}G_{ds}^{m}C_{ds}A_{ds}T_{ds}^{m}C_{ds}T_{ds}^{m}C_{ds}Tₖₛ^{m}Cₖₛ^{m}Cₖ | 23 |

A subscript “d” indicates an unmodified, 2′-β-D-deoxyribosyl sugar moiety.
A subscript “e” indicates a 2′-MOE-β-D-ribofuranosyl sugar moiety.
A subscript “m” indicates a 2′-O-methyl-β-D-ribofuranosyl sugar moiety.
A subscript “k” indicates a cEt.
A subscript “s” indicates a phosphorothioate internucleoside linkage.
A subscript “f” indicates a 2′-F-β-D-2′-deoxyribosyl sugar moiety.
A subscript “(FANA)” indicates a 2′-F-β-D-arabinofiiranosyl modified sugar moiety.
A superscript “m” before a C indicates 5-methyl Cytosine.

### Experimental Procedures & Results

For in vitro toxicity studies, 3T3-L1 cells were electroporated with 27, 80, 250, 740, 2, 222, 6, 667, or 20,000 nM of modified oligonucleotide and levels of Raptor were measured by RT-PCR as in Example 1.

For in vivo toxicity studies, two BALB/C mice per group were administered 100 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

TABLE 8b
Toxicity of modified oligonucleotides complementary Factor XI

| | position | | | |
|---|---|---|---|---|
| | of altered | sugar | | |
| | nucleotide | modification | Raptor | ALT at |
| Compound | in central | of altered | IC50 | 100 mg/kg |
| ID | region | nucleotide | (μM) | (IU/L) |
| 464917 | n/a | n/a | 1.6 | 18751 * |
| 982034 | 2 | 2′-OMe | >20 | 1363 |
| 985292 | 2 | 2′-MOE | 15.5 | 2406 |
| 985293 | 2 | cEt | 9.3 | 15141 |
| 1011274 | 2 | 2′-FANA | 2.3 | death |
| 604581 | 2 | 2′-ribo-F | 6 | 14957 |
| 982035 | 3 | 2′-OMe | 1.8 | 6411 |
| 985294 | 3 | 2′-MOE | 6.2 | 2836 |
| 985295 | 3 | cEt | 5.2 | 3669 |
| 1011276 | 3 | 2′-FANA | >20 | death |
| 605933 | 3 | 2′-ribo-F | 4.6 | 18570 |

* ALT for 464917 is for a 50 mg/kg dose

For the in vitro study reported in the tables below, 3T3-L1 cells were electroporated with 27, 80, 250, 740, 2, 222, 6,667, or 20,000 nM of modified oligonucleotide and levels of P21 and Gadd45a were measured by RT-qPCR. Levels of Gadd45a were analyzed using primer probe set Mm00432802_m1 (ThermoFisher).

TABLE 8c
in vitro P21 Expression in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 464917 | 108 | 124 | 122 | 169 | 228 | 478 | 749 |
| 982033 | 119 | 120 | 128 | 128 | 218 | 498 | 895 |
| 982034 | 115 | 121 | 110 | 102 | 136 | 266 | 840 |
| 982035 | 162 | 157 | 175 | 206 | 466 | 768 | 661 |

TABLE 8d
in vitro Gadd45a Expression in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 464917 | 130 | 150 | 115 | 179 | 321 | 632 | 633 |
| 982033 | 120 | 117 | 126 | 203 | 331 | 767 | 798 |
| 982034 | 89 | 111 | 103 | 102 | 173 | 678 | 800 |
| 982035 | 161 | 120 | 140 | 181 | 557 | 779 | 497 |

For the in vitro study reported in the tables below, b.END cells were electroporated with 27, 80, 250, 740, 2, 222, 6,667, or 20,000 nM of modified oligonucleotide and levels of P21 and Gadd45a were measured by RT-qPCR. Levels of Gadd45a were analyzed using primer probe set Mm00432802_m1 (ThermoFisher). Levels of P21 were analyzed using primer probe set Mm04207341_m1 (ThermoFisher).

TABLE 8e
in vitro P21 Expression in b.END cells

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 464917 | 111 | 115 | 124 | 120 | 139 | 192 | 446 |
| 982033 | 105 | 102 | 107 | 110 | 125 | 171 | 414 |
| 982034 | 106 | 102 | 109 | 112 | 120 | 132 | 208 |
| 982035 | 102 | 97 | 111 | 115 | 129 | 168 | 392 |

TABLE 8f
in vitro Gadd45a Expression in b.END cells

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 464917 | 101 | 83 | 85 | 87 | 129 | 236 | 380 |
| 982033 | 89 | 76 | 101 | 91 | 177 | 347 | 731 |
| 982034 | 58 | 73 | 86 | 88 | 115 | 202 | 373 |
| 982035 | 68 | 72 | 81 | 103 | 166 | 298 | 620 |

For the in vivo activity study in the tables below, 3 BALB/C mice per group were administered 3.37, 11, 33, or 100 mg/kg doses of modified oligonucleotide, as indicated in the table below, by subcutaneous injection and sacrificed 72 hours later. Liver mRNA was isolated an analyzed by RT-PCR as described in Example 1 above. Expression levels were normalized with Ribogreen® and are expressed relative to PBS-treated control mice.

TABLE 8g
Activity and toxicity of modified
oligonucleotides complementary FXI

| | position | | | |
|---|---|---|---|---|
| | of altered | sugar | | |
| | nucleotide | modification | | |
| Compound | in central | of altered | ALT (IU/L) | ALT (IU/L) |
| ID | region | nucleotide | at 100 mg/kg | at 33 mg/kg |
| 464917 | n/a | n/a | 11816 | 11682 |
| 982033 | 1 | OMe | 26992 | 3951 |
| 982034 | 2 | OMe | 7954 | 920 |
| 982035 | 3 | OMe | 28994 | 3848 |

TABLE 8h
In Vivo Dose-response of FXI mRNA upon treatment with
modified oligonucleotides complementary to FXI

| Compound | 3.37 mg/kg | 11 mg/kg | 33 mg/kg | 100 mg/kg |
|---|---|---|---|---|
| ID | Expression level of FXI mRNA (% Control) | | | |
| 464917 | 47 | 12 | 8.0 | 1.8 |
| 982033 | 53 | 18 | 10 | 5.5 |
| 982034 | 53 | 24 | 9.9 | 3.5 |
| 982035 | 36 | 20 | 11 | 5.3 |

TABLE 8i
In Vivo Dose-response of P21 mRNA upon treatment with
modified oligonucleotides complementary to FXI

| Compound | 3.37 mg/kg | 11 mg/kg | 33 mg/kg | 100 mg/kg |
|---|---|---|---|---|
| ID | Expression level of P21 mRNA (% Control) | | | |
| 464917 | 230 | 4143 | 4678 | 5289 |
| 982033 | 122 | 1106 | 2926 | 5653 |
| 982034 | 93 | 297 | 1694 | 4294 |
| 982035 | 418 | 1283 | 4759 | 6960 |

### Example 4 Effect of Position-Specific 2′ and 5′-Modifications on In Vivo Activity and Liver Toxicity of Modified Oligonucleotides Complementary to Factor XI

Modified oligonucleotides were synthesized with 2′ or 5′ modifications as indicated in the table below. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleobases in the central region. The compounds in the table below are 100% complementary to mouse Factor XI, the complement of GENBANK NT_039460.6 truncated from 6086000 to 6111000 (SEQ ID NO: 2), at position 11699 to 11714.

TABLE 9
Modified oligonucleotides complementary to Factor XI

| | position of | | | |
|---|---|---|---|---|
| | altered | sugar | | |
| | nucleotide | modification of | | SEQ |
| Compound | in central | altered | | ID |
| ID | region | nucleotide | Chemistry Notation (5′-3′) | NO |
| 464917 | n/a | n/a | GₖₛTₖₛ^{m}CₖₛT_{ds}G_{ds}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 443919 | n/a | n/a | GₑₛTₑₛ^{m}CₑₛT_{ds}G_{ds}T_{ds}G_{ds}^{m}C_{ds}A_{ds}T_{ds}^{m}C_{ds}T_{ds}^{m}C_{ds}Tₑₛ^{m}Cₑₛ^{m}Cₑ | 22 |
| 465977 | n/a | n/a | GₖₛTₖₛ^{m}CₖₛT_{ds}G_{ds}T_{ds}G_{ds}^{m}C_{ds}A_{ds}T_{ds}^{m}C_{ds}T_{ds}^{m}C_{ds}Tₑₛ^{m}Cₑₛ^{m}Cₑ | 22 |
| 483706 | n/a | n/a | GₑₛTₑₛ^{m}CₑₛT_{ds}G_{ds}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1183062 | 2 | 5′-(R)-Me | GₖₛTₖₛ^{m}CₖₛT_{ds}G_{[(R)-μ]s}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1183059 | 2 | 5′-(S)-Me | GₖₛTₖₛ^{m}CₖₛT_{ds}G_{[(S)-μ]s}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1183065 | 2 | 5′-(R)-allyl | GₖₛTₖₛ^{m}CₖₛT_{ds}G_{[(R)-γ]s}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1183063 | 3 | 5′-(R)-Me | GₖₛTₖₛ^{m}CₖₛT_{ds}G_{ds}T_{[(R)-μ]s}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1183060 | 3 | 5′-(S)-Me | GₖₛTₖₛ^{m}CₖₛT_{ds}G_{ds}T_{[(S)-μ]s}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1183066 | 3 | 5′-(R)-allyl | GₖₛTₖₛ^{m}CₖₛT_{ds}G_{ds}T_{[(R)-γ]s}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 1183064 | 4 | 5′-(R)-Me | GₖₛTₖₛ^{m}CₖₛT_{ds}G_{ds}T_{ds}G_{[(R)-μ]s}^{m}C_{ds}A_{ds}T_{ds}^{m}C_{ds}T_{ds}^{m}C_{ds}Tₖₛ^{m}Cₖₛ^{m}Cₖ | 22 |
| 1183061 | 4 | 5′-(S)-Me | GₖₛTₖₛ^{m}CₖₛT_{ds}G_{ds}T_{ds}G_{[(S)-μ]s}^{m}C_{ds}A_{ds}T_{ds}^{m}C_{ds}T_{ds}^{m}C_{ds}Tₖₛ^{m}Cₖₛ^{m}Cₖ | 22 |
| 1183067 | 4 | 5′-(R)-allyl | GₖₛTₖₛ^{m}CₖₛT_{ds}G_{ds}T_{ds}G_{[(R)-γ]s}^{m}C_{ds}A_{ds}T_{ds}^{m}C_{ds}T_{ds}^{m}C_{ds}Tₖₛ^{m}Cₖₛ^{m}Cₖ | 22 |

A subscript “d” indicates an unmodified, 2′-β-D-deoxyribosyl sugar moiety.
A subscript “k” indicates a cEt.
A subscript “s” indicates a phosphorothioate internucleoside linkage.
A subscript “[μ]” indicates a 5′-(R,S)-Me-β-D-2’-deoxyribosyl sugar moiety.
A subscript “[(R)-μ]” indicates a 5′-(R)-Me-β-D-2’-deoxyribosyl sugar moiety.
A subscript “[(S)-μ]” indicates a 5′-(S)-Me-β-D-2′-deoxyribosyl sugar moiety.
A subscript “[γ]” indicates a 5′-(R,S)-allyl-β-D-2′-deoxyribosyl sugar moiety.
A subscript “[(R)-γ]” indicates a 5′-(R)-allyl-P-β-2’-deoxyribosyl sugar moiety.
A subscript “[(S)-γ]” indicates a 5′-(S)-allyl-β-D-2′-deoxyribosyl sugar moiety.
A subscript “[ε]” indicates a 5′-(R,S)-ethyl-β-D-2′-deoxyribosyl sugar moiety.
A subscript “[(R)-ε]” indicates a 5′-(R)-ethyl-β-D-2′-deoxyribosyl sugar moiety.
A subscript “[(S)-ε]” indicates a 5′-(S)-ethyl-β-D-2’-deoxyribosyl sugar moiety.

### Experimental Procedures & Results

For the in vivo activity and toxicity study below, two BALB/C mice per group were administered 33 or 100 mg/kg modified oligonucleotide by subcutaneous injection and sacrificed 72 hours later. Expression levels of FXI were measured by RT-qPCR using primer probe set RTS2898 (forward sequence: ACATGACAGGCGCGATCTCT, SEQ ID NO: 78; reverse sequence: TCTAGGTTCACGTACACATCTTTGC, SEQ ID NO: 79; probe sequence: TTCCTTCAAGCAATGCCCTCAGCAAT, SEQ ID NO: 80). Plasma levels of ALT were measured using an automated clinical chemistry analyzer. Increased ALT is indicative of acute liver toxicity.

TABLE 10
Toxicity and activity of modified oligonucleotides complementary Factor XI

| | position | | | | | |
|---|---|---|---|---|---|---|
| | of altered | sugar | | | | |
| | nucleotide | modification | FXI mRNA | FXI mRNA | ALT | ALT |
| Compound | in central | of altered | (% control) | (% control) | (IU/L) at | (IU/L) at |
| ID | region | nucleotide | 33 mg/kg | 100 mg/kg | 33 mg/kg | 100 mg/kg |
| 464917 | n/a | n/a | 11 | 0.9 | 7511 | 31066* |
| 443919 | n/a | n/a | 27 | 7.9 | 24 | 57 |
| 465977 | n/a | n/a | 5.6 | n.d. | 11575 | death |
| 483706 | n/a | n/a | 20.3 | 4.9 | 52 | 732 |
| 1183062 | 2 | 5′-(R)-Me | 5.7 | n.d. | 12083 | death |
| 1183059 | 2 | 5′-(S)-Me | 4.0 | 2.4 | 662 | 7894 |
| 1183065 | 2 | 5′-(R)-allyl | 5.2 | 1.5 | 4707 | 24000 |
| 1183063 | 3 | 5′-(R)-Me | 4.9 | 2.0 | 2458 | 14891 |
| 1183060 | 3 | 5′-(S)-Me | 8.2 | 2.1 | 8710 | 23995 |
| 1183066 | 3 | 5′-(R)-allyl | 5.1 | 2.0 | 524 | 6473 |
| 1183064 | 4 | 5′-(R)-Me | 4.0 | 1.5 | 4357 | 11342 |
| 1183061 | 4 | 5′-(S)-Me | 4.1 | 2.3 | 1891 | 20557 |
| 1183067 | 4 | 5′-(R)-allyl | 11 | 3.6 | 184 | 2536 |

*One of two mice died

For the in vitro study reported in the tables below, 3T3-L1 cells were electroporated with 27 nM, 80 nM, 250 nM, 740 nM, 2, 222 nM, 6,667 nM, or 20,000 nM of modified oligonucleotide and levels of P21 and Gadd45a were measured by RT-qPCR as described in Example 1 above. Caspase-3 and caspase-7 activation was measured using the Caspase-Glo® 3/7 Assay System (G8090, Promega). Levels of caspase activation correlate with apoptotic cell death.

TABLE 10b
in vitro P21 Expression in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 464917 | 100 | 100 | 116 | 139 | 216 | 496 | 1232 |
| 443919 | 122 | 116 | 99 | 86 | 114 | 105 | 184 |
| 465977 | 104 | 117 | 103 | 106 | 139 | 220 | 578 |
| 483706 | 105 | 92 | 116 | 125 | 135 | 165 | 376 |

TABLE 10c
in vitro Gadd45a Expression in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 464917 | 89 | 93 | 106 | 113 | 157 | 324 | 599 |
| 443919 | 163 | 166 | 147 | 129 | 145 | 126 | 178 |
| 465977 | 101 | 110 | 119 | 100 | 135 | 150 | 334 |
| 483706 | 89 | 133 | 185 | 194 | 197 | 217 | 459 |

TABLE 10d
in vitro Caspase Activation in 3T3-L1 cells

| Compound | 27 nM | 80 nM | 250 nM | 740 nM | 2,222 nM | 6,667 nM | 20,000 nM |
|---|---|---|---|---|---|---|---|
| ID | Caspase Activation | | | | | | |
| 464917 | 4388 | 4428 | 4656 | 6208 | 20274 | 48106 | 82324 |
| 443919 | 4200 | 4802 | 4986 | 4605 | 4714 | 4552 | 9385 |
| 465977 | 4017 | 4133 | 4253 | 4465 | 6983 | 34156 | 61008 |
| 483706 | 4155 | 4595 | 4020 | 4476 | 4585 | 6565 | 16766 |

For the in vivo study in the table below, three BALB/C mice per group were administered 11 or 33 mg/kg of modified oligonucleotide and sacrificed after 72 hours.

TABLE 10e
in vivo Activity and toxicity of modified oligonucleotides complementary FXI

| | 2′ sugar | 2′ sugar | | | | |
|---|---|---|---|---|---|---|
| | modification | modification | P21 mRNA | Tnfrsf10b mRNA | FXI mRNA | |
| Compound | of nucleosides | of nucleosides | @ 33 mg/kg | @ 33 mg/kg | @ 33 mg/kg | ALT@33 |
| ID | in 5′ region | in 3′ region | (% control) | (% control) | (% control) | mg/kg |
| 464917 | kkk | kkk | 24040 | 108884 | 1.4 | 18316 |
| 443919 | kkk | eee | 109 | 110 | 16 | 68 |
| 465977 | eee | kkk | n.d. | n.d. | n.d. | death |
| 483706 | eee | eee | 1195 | 733 | 2.7 | 1424 |

### Example 5 Effect of Position-Specific 2′-Modifications on In Vitro Activity and In Vivo Liver Toxicity of Modified Oligonucleotides Complementary to PTEN

Modified oligonucleotides were synthesized with 2′ modifications as indicated in the table below. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleobases in the central region. The compounds in the table below are 10000 complementary to mouse PTEN, GENBANK NC_000085.6, truncated from 32755001 to 32829000 (SEQ ID NO: 3), at position 2635 to 2650.

TABLE 11
Modified oligonucleotides complementary to PTEN

| | position | | | |
|---|---|---|---|---|
| | of altered | sugar | | |
| | nucleotide | modification | | SEQ |
| Compound | in central | of altered | | ID |
| ID | region | nucleotide | Chemistry Notation (5′-3′) | NO |
| 482050 | n/a | n/a | AₖₛTₖₛ^{m}CₖₛA_{ds}T_{ds}G_{ds}G_{ds}^{m}C_{ds}T_{ds}G_{ds}^{m}C_{ds}A_{ds}G_{ds}^{m}CₖₛTₖₛTₖ | 24 |
| 982036 | 1 | 2′-OMe | AₖₛTₖₛ^{m}CₖₛAₘₛT_{ds}G_{ds}G_{ds}^{m}C_{ds}T_{ds}G_{ds}^{m}C_{ds}A_{ds}G_{ds}^{m}CₖₛTₖₛTₖ | 24 |
| 982037 | 2 | 2′-OMe | AₖₛTₖₛ^{m}CₖₛA_{ds}UₘₛG_{ds}G_{ds}^{m}C_{ds}T_{ds}G_{ds}^{m}C_{ds}A_{ds}G_{ds}^{m}CₖₛTₖₛTₖ | 25 |
| 985297 | 2 | 2′-MOE | AₖₛTₖₛ^{m}CₖₛA_{ds}Te_{S}G_{ds}G_{ds}^{m}C_{ds}T_{ds}G_{ds}^{m}C_{ds}A_{ds}G_{ds}^{m}CₖₛTₖₛTₖ | 24 |
| 985298 | 2 | cEt | AₖₛTₖₛ^{m}CₖₛA_{ds}TₖₛG_{ds}G_{ds}^{m}C_{ds}T_{ds}G_{ds}^{m}C_{ds}A_{ds}G_{ds}^{m}CₖₛTₖₛTₖ | 24 |
| 1011277 | 2 | 2′-FANA | AₖₛTₖₛ^{m}CₖₛA_{ds}U_{(FANA)s}G_{ds}G_{ds}^{m}C_{ds}T_{ds}G_{ds}^{m}C_{ds}A_{ds}G_{ds}^{m}CₖₛTₖₛTₖ | 25 |
| 985296 | 2 | 2′-ribo-F | AₖₛTₖₛ^{m}CₖₛA_{ds}U_{fS}G_{ds}G_{ds}^{m}C_{ds}T_{ds}G_{ds}^{m}C_{ds}A_{ds}G_{ds}^{m}CₖₛTₖₛTₖ | 25 |
| 982038 | 3 | 2′-OMe | AₖₛTₖₛ^{m}CₖₛA_{ds}T_{ds}GₘₛG_{ds}^{m}C_{ds}T_{ds}G_{ds}^{m}C_{ds}A_{ds}G_{ds}^{m}CₖₛTₖₛTₖ | 24 |
| 985301 | 3 | 2′-MOE | AₖₛTₖₛ^{m}CₖₛA_{ds}T_{ds}GₑₛG_{ds}^{m}C_{ds}T_{ds}G_{ds}^{m}C_{ds}A_{ds}G_{ds}^{m}CₖₛTₖₛTₖ | 24 |
| 985302 | 3 | cEt | AₖₛTₖₛ^{m}CₖₛA_{ds}T_{ds}GₖₛG_{ds}^{m}C_{ds}T_{ds}G_{ds}^{m}C_{ds}A_{ds}G_{ds}^{m}CₖₛTₖₛTₖ | 24 |
| 1011278 | 3 | 2′-FANA | AₖₛTₖₛ^{m}CₖₛA_{ds}T_{ds}G_{(FANA)s}G_{ds}^{m}C_{ds}T_{ds}G_{ds}^{m}C_{ds}A_{ds}G_{ds}^{m}CₖₛTₖₛTₖ | 24 |
| 985300 | 3 | 2′-ribo-F | AₖₛTₖₛ^{m}CₖₛA_{ds}T_{ds}G_{fs}G_{ds}^{m}C_{ds}T_{ds}G_{ds}^{m}C_{ds}A_{ds}G_{ds}^{m}CₖₛTₖₛTₖ | 24 |

A subscript “d” indicates an unmodified, 2′-β-D-deoxyribosyl sugar moiety.
A subscript “e” indicates a 2′-MOE-β-D-ribofuranosyl sugar moiety.
A subscript “m” indicates a 2′-O-methyl-β-D-ribofiiranosyl sugar moiety.
A subscript “k” indicates a cEt.
A subscript “s” indicates a phosphorothioate internucleoside linkage.
A subscript ″f″ indicates a 2′-F-β-D-2′-deoxyribosyl sugar moiety.
A subscript “(FANA)” indicates a 2′-F-β-D-arabinofuranosyl modified sugar moiety.
A superscript “m” before a C indicates 5-methyl Cytosine.

For in vitro activity studies, 3T3-L1 cells were plated and transfected with 16, 80, 400, 2,000, and 10,000 nM modified oligonucleotide by electroporation. mRNA was harvested and analyzed by RT-qPCR. PTEN mRNA was detected and RAPTOR mRNA was detected.

For in vivo toxicity studies, 2-4 BALB/C mice per group were administered 200 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

TABLE 12
Activity and toxicity of modified
oligonucleotides complementary PTEN

| | position | | | | |
|---|---|---|---|---|---|
| | of altered | sugar | | | |
| | nucleotide | modification | PTEN | RAPTOR | ALT @ |
| Compound | in central | of altered | IC₅₀ | IC₅₀ | 200 mg/kg |
| ID | region | nucleotide | (μM) | (μM) | (IU/L) |
| 482050 | n/a | n/a | 3.9 | 2.4 | 2458 |
| 982037 | 2 | 2′-OMe | 2.7 | 10 | 133 |
| 985297 | 2 | 2′-MOE | 3 | 9.4 | 242 |
| 985298 | 2 | cEt | 1.4 | 2.1 | 890 |
| 1011277 | 2 | 2′-FANA | 3.1 | 3.5 | 1488 |
| 985296 | 2 | 2′-ribo-F | 2.2 | 6 | 1884 |
| 982038 | 3 | 2′-OMe | 1.8 | 3.7 | 327 |
| 985301 | 3 | 2′-MOE | 1.5 | 5 | 261 |
| 985302 | 3 | cEt | 2 | 3.3 | 87 |
| 1011278 | 3 | 2′-FANA | 1.7 | 1.1 | 14073 |
| 985300 | 3 | 2′-ribo-F | 2.2 | 6 | 107 |

For the in vitro study reported in the tables below, 3T3-L1 cells were transfected with 27, 80, 250, 740, 2, 222, 6,667, or 20,000 nM of modified oligonucleotide by electroporation and levels of P21 and Gadd45a were measured by RT-qPCR as described in example 1.

TABLE 12b
in vitro P21 Expression

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 482050 | 111 | 107 | 113 | 124 | 113 | 130 | 157 |
| 982036 | 92 | 95 | 93 | 95 | 91 | 110 | 162 |
| 982037 | 112 | 108 | 99 | 105 | 112 | 120 | 113 |
| 982038 | 108 | 105 | 111 | 111 | 114 | 99 | 108 |

TABLE 12c
in vitro Gadd45a Expression

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 482050 | 65 | 64 | 70 | 67 | 79 | 176 | 276 |
| 982036 | 81 | 62 | 62 | 71 | 113 | 189 | 467 |
| 982037 | 107 | 90 | 79 | 75 | 79 | 100 | 165 |
| 982038 | 110 | 112 | 104 | 131 | 118 | 129 | 266 |

### Example 6 Effect of Position-Specific 2′-Modifications on In Vitro Activity and In Vivo Liver Toxicity of Modified Oligonucleotides Complementary to SOD1

Modified oligonucleotides were synthesized with 2′ modifications as indicated in the table below. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleobases in the central region. The compounds in the table below are 100% complementary to mouse SOD1, GENBANK NT_039625.7 truncated from 24924000 to 24933000 (SEQ ID NO: 4), at position 5685 to 5880.

TABLE 13
Modified oligonucleotides complementary to SOD1

| | position of | | | |
|---|---|---|---|---|
| | altered | sugar | | |
| | nucleotide | modification | | SEQ |
| Compound | in central | of altered | | ID |
| ID | region | nucleotide | Chemistry Notation (5′-3′) | NO |
| 508031 | n/a | n/a | TₖₛGₖₛAₖₛG_{ds}G_{ds}T_{ds}^{m}C_{ds}^{m}C_{ds}T_{ds}G_{ds}^{m}C_{ds}A_{ds}^{m}C_{ds}TₖₛGₖₛGₖ | 26 |
| 508034 | n/a | n/a | TₖₛGₖₛAₖₛG_{ds}G_{ds}T_{ds}^{m}C_{ds}^{m}C_{ds}T_{ds}G_{ds}^{m}C_{ds}A_{ds}^{m}C_{ds}TₑₛGₑₛGₑ | 26 |
| 508037 | n/a | n/a | TₑₛGₑₛAₑₛG_{ds}G_{ds}T_{ds}^{m}C_{ds}^{m}C_{ds}T_{ds}G_{ds}^{m}C_{ds}A_{ds}^{m}C_{ds}TₖₛGₖₛGₖ | 26 |
| 529933 | n/a | n/a | TₑₛGₑₛAₑₛG_{ds}G_{ds}T_{ds}^{m}C_{ds}^{m}C_{ds}T_{ds}G_{ds}^{m}C_{ds}A_{ds}^{m}C_{ds}TₑₛGₑₛGₑ | 26 |
| 895154 | 1 | 2′-OMe | TₖₛGₖₛAₖₛGₘₛG_{ds}T_{ds}^{m}C_{ds}^{m}C_{ds}T_{ds}G_{ds}^{m}C_{ds}A_{ds}^{m}C_{ds}TₖₛGₖₛGₖ | 26 |
| 895155 | 2 | 2′-OMe | TₖₛGₖₛAₖₛG_{ds}GₘₛT_{ds}^{m}C_{ds}^{m}C_{ds}T_{ds}G_{ds}^{m}C_{ds}A_{ds}^{m}C_{ds}TₖₛGₖₛGₖ | 26 |
| 985305 | 2 | 2′-MOE | TₖₛGₖₛAₖₛG_{ds}GₑₛT_{ds}^{m}C_{ds}^{m}C_{ds}T_{ds}G_{ds}^{m}C_{ds}A_{ds}^{m}C_{ds}TₖₛGₖₛGₖ | 26 |
| 985306 | 2 | cEt | TₖₛGₖₛAₖₛG_{ds}GₖₛT_{ds}^{m}C_{ds}^{m}C_{ds}T_{ds}G_{ds}^{m}C_{ds}A_{ds}^{m}C_{ds}TₖₛGₖₛGₖ | 26 |
| 1011279 | 2 | 2′-FANA | TₖₛGₖₛAₖₛG_{ds}G_{(FANA)s}T_{ds}^{m}C_{ds}^{m}C_{ds}T_{ds}G_{ds}^{m}C_{ds}A_{ds}^{m}C_{ds}TₖₛGₖₛGₖ | 26 |
| 985304 | 2 | 2′-ribo-F | TₖₛGₖₛAₖₛG_{ds}G_{fs}T_{ds}^{m}C_{ds}^{m}C_{ds}T_{ds}G_{ds}^{m}C_{ds}A_{ds}^{m}C_{ds}TₖₛGₖₛGₖ | 26 |
| 895156 | 3 | 2′-OMe | TₖₛGₖₛAₖₛG_{ds}G_{ds}Uₘₛ^{m}C_{ds}^{m}C_{ds}T_{ds}G_{ds}^{m}C_{ds}A_{ds}^{m}C_{ds}TₖₛGₖₛGₖ | 27 |
| 985309 | 3 | 2′-MOE | TₖₛGₖₛAₖₛG_{ds}G_{ds}Tₑₛ^{m}C_{ds}^{m}C_{ds}T_{ds}G_{ds}^{m}C_{ds}A_{ds}^{m}C_{ds}TₖₛGₖₛGₖ | 26 |
| 985310 | 3 | cEt | TₖₛGₖₛAₖₛG_{ds}G_{ds}Tₖₛ^{m}C_{ds}^{m}C_{ds}T_{ds}G_{ds}^{m}C_{ds}A_{ds}^{m}C_{ds}TₖₛGₖₛGₖ | 26 |
| 1011280 | 3 | 2′-FANA | TₖₛGₖₛAₖₛG_{ds}G_{ds}U_{(FANA)s}^{m}C_{ds}^{m}C_{ds}T_{ds}G_{ds}^{m}C_{ds}A_{ds}^{m}C_{ds}TₖₛGₖₛGₖ | 27 |
| 985308 | 3 | 2′-ribo-F | TₖₛGₖₛAₖₛG_{ds}G_{ds}U_{fs}^{m}C_{ds}^{m}C_{ds}T_{ds}G_{ds}^{m}C_{ds}A_{ds}^{m}C_{ds}TₖₛGₖₛGₖ | 27 |

A subscript “d” indicates an unmodified, 2′-β-D-deoxyribosyl sugar moiety.
A subscript “e” indicates a 2′-MOE-β-D-ribofuranosyl sugar moiety.
A subscript “m” indicates a 2′-O-methyl-β-D-ribofuranosyl sugar moiety.
A subscript “k” indicates a cEt.
A subscript “s” indicates a phosphorothioate internucleoside linkage.
A subscript “f” indicates a 2′-F-β-D-2′-deoxyribosyl sugar moiety.
A subscript “(FANA)” indicates a 2′-F-β-D-arabinofuranosyl modified sugar moiety.
A superscript “m” before a C indicates 5-methyl Cytosine.

For in vitro activity studies, 3T3-L1 cells were plated at 20,000 cells/well and transfected with 16, 80, 400, 2,000, and 10,000 nM modified oligonucleotide by electroporation. mRNA was harvested and analyzed by RT-qPCR. SOD1 mRNA was detected using primer probe set RTS3025 (forward sequence: TTTTTTGCGCGGTCCTTTC (SEQ ID NO: 119); reverse sequence: GAGGGACCAGAGAGAGCAAGAC (SEQ ID NO: 120); probe sequence: CGCCTTCCGTCCGTCGGCT (SEQ ID NO:121)) and RAPTOR mRNA was detected as in Example 1 above.

For the in vivo toxicity study in the table below, two BALB/C mice per modified oligonucleotide were administered 150 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

TABLE 14
Activity and toxicity of modified
oligonucleotides complementary SOD1

| | position | | | | |
|---|---|---|---|---|---|
| | of altered | sugar | | | |
| | nucleotide | modification | SOD1 | RAPTOR | ALT @ |
| Compound | in central | of altered | IC₅₀ | IC₅₀ | 150 mg/kg |
| ID | region | nucleotide | (μM) | (μM) | (IU/L) |
| 508031 | n/a | n/a | 0.03 | 0.46 | 21934 |
| 895155 | 2 | 2′-OMe | 0.04 | 1 | 112 |
| 985305 | 2 | 2′-MOE | 0.21 | n/a | 63 |
| 985306 | 2 | cEt | 1.61 | 10.2 | 826 |
| 1011279 | 2 | 2′-FANA | 0.28 | 1 | death |
| 985304 | 2 | 2′-ribo-F | 0.04 | 0.8 | 182 |
| 895156 | 3 | 2′-OMe | 0.48 | 4.5 | 1371 |
| 985309 | 3 | 2′-MOE | 0.61 | 6 | 1629 |
| 985310 | 3 | cEt | 1.46 | 11.9 | 178 |
| 1011280 | 3 | 2′-FANA | 0.6 | 4 | death |
| 985308 | 3 | 2′-ribo-F | 0.24 | 0.92 | 887 |

For in vitro activity studies, 3T3-L1 cells were plated at 20,000 cells/well and transfected with 30 nM, 80 nM, 250 nM, 740 nM, 2,222 nM, 6,667 nM, or 20,000 nM modified oligonucleotide by electroporation. P21 and Gadd45a mRNA were analyzed as in Example 1 above and caspase activation was measured as in Example 4 above. Results were normalized with Ribogreen® and are presented relative to the average of untreated control cells.

TABLE 14b
in vitro P21 Expression

| Compound | 30 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 508031 | 104 | 96 | 104 | 91 | 99 | 180 | 366 |
| 895154 | 94 | 117 | 85 | 93 | 105 | 159 | 181 |
| 895155 | 98 | 110 | 92 | 88 | 88 | 101 | 137 |
| 895156 | 95 | 104 | 74 | 97 | 125 | 139 | 283 |

TABLE 14c
in vitro Gadd45a Expression

| Compound | 30 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 508031 | 103 | 99 | 113 | 103 | 139 | 564 | 844 |
| 895154 | 110 | 125 | 114 | 106 | 130 | 297 | 669 |
| 895155 | 129 | 139 | 120 | 126 | 122 | 145 | 340 |
| 895156 | 122 | 132 | 94 | 125 | 223 | 490 | 856 |

TABLE 14d
in vitro Caspase Activation in 3T3-L1 cells

| Compound | 27 nM | 80 nM | 250 nM | 740 nM | 2,222 nM | 6,667 nM | 20,000 nM |
|---|---|---|---|---|---|---|---|
| ID | Caspase Activation | | | | | | |
| 508031 | 10871 | 11667 | 12107 | 14458 | 46619 | 101512 | 177873 |
| 895154 | 11681 | 11503 | 11656 | 11422 | 17167 | 70398 | 124774 |
| 895155 | 11669 | 11005 | 11479 | 11156 | 12487 | 20199 | 77630 |
| 895156 | 11980 | 10646 | 10616 | 11178 | 24226 | 72844 | 153302 |

For the in vivo toxicity study in the table below, three BALB/C mice per modified oligonucleotide were administered 100 mg/kg modified oligonucleotide by subcutaneous injection and sacrificed 72 hours later. One animal was administered an injection of saline as a control. Plasma levels of ALT and AST were measured using an automated clinical chemistry analyzer. Increased ALT and AST are indicative of acute liver toxicity.

For the in vivo study in the table below, three BALB/C mice per group were administered 33 or 100 mg/kg of modified oligonucleotide and sacrificed after 24 hours.

TABLE 15
Activity and toxicity of modified oligonucleotides complementary SOD1

| | position | | | | | |
|---|---|---|---|---|---|---|
| | of altered | sugar | | | | |
| | nucleotide | modification | P21 mRNA | Tnfrsf10b mRNA | Gadd45a mRNA | SOD1 mRNA |
| Compound | in central | of altered | @ 100 mg/kg | @ 100 mg/kg | @ 100 mg/kg | @ 100 mg/kg |
| ID | region | nucleotide | (% control) | (% control) | (% control) | (% control) |
| 508031 | n/a | n/a | 823 | 399 | 321 | 36 |
| 895154 | 1 | 2′-OMe | 125 | 176 | 345 | 56 |
| 895155 | 2 | 2′-OMe | 67 | 147 | 365 | 75 |
| 895156 | 3 | 2′-OMe | 538 | 351 | 525 | 51 |

For the in vivo study in the table below, three BALB/C mice per group were administered 33 or 100 mg/kg of modified oligonucleotide and sacrificed after 72 hours.

TABLE 15b
Activity and toxicity of modified oligonucleotides complementary SOD1

| | position | | | | | | |
|---|---|---|---|---|---|---|---|
| | of altered | sugar | | | | | |
| | nucleotide | modification | P21 mRNA | Tnfrsf10b mRNA | Gadd45a mRNA | SOD1 mRNA | |
| Compound | in central | of altered | @ 100 mg/kg | @ 100 mg/kg | @ 100 mg/kg | @ 100 mg/kg | ALT @ 100 |
| ID | region | nucleotide | (% control) | (% control) | (% control) | (% control) | mg/kg (IU/L) |
| 508031 | n/a | n/a | 6007 | 9032 | 392 | 13 | 16,317 |
| 895154 | 1 | 2′-OMe | 561 | 1042 | 129 | 19 | 206 |
| 895155 | 2 | 2′-OMe | 165 | 233 | 18 | 26 | 41 |
| 895156 | 3 | 2′-OMe | 3218 | 8189 | 190 | 9.6 | 1,242 |

TABLE 15c
Activity and toxicity of modified oligonucleotides complementary to SOD1

| | 2′ sugar | 2′ sugar | P21 mRNA | Tnfrsf10b mRNA | SOD1 mRNA | |
|---|---|---|---|---|---|---|
| Compound | modification | modification | @ 100 mg/kg | @ 100 mg/kg | @ 100 mg/kg | ALT @ 100 |
| ID | in 5′ region | in 3′ region | (% control) | (% control) | (% control) | mg/kg (IU/L) |
| 508031 | kkk | kkk | 3478 | 4593 | 9 | 14526 |
| 508034 | kkk | eee | 11365 | 7288 | 3 | 22396* |
| 508037 | eee | kkk | 130 | 225 | 17 | 20 |
| 529933 | eee | eee | 90 | 142 | 18 | 11 |

*2/3 animals were found dead

For the in vitro study reported in the tables below, 3T3-L1 cells were electroporated with 27 nM, 80 nM, 250 nM, 740 nM, 2, 222 nM, 6,667 nM, or 20,000 nM of modified oligonucleotide and levels of P21 and Gadd45a were measured by RT-qPCR as described in Example 1 above. Caspase-3 and caspase-7 activation was measured using the Caspase-Glo® 3/7 Assay System (G8090, Promega). Levels of caspase activation correlate with apoptotic cell death.

TABLE 15d
in vitro Caspase Activation in 3T3-L1 cells

| Compound | 27 nM | 80 nM | 250 nM | 740 nM | 2,222 nM | 6,667 nM | 20,000 nM |
|---|---|---|---|---|---|---|---|
| ID | Caspase Activation | | | | | | |
| 508031 | 5969 | 6550 | 5986 | 8376 | 22499 | 56695 | 91450 |
| 508034 | 5652 | 5258 | 6555 | 7590 | 17098 | 49473 | 73813 |
| 508037 | 4027 | 4000 | 4222 | 4104 | 4208 | 3899 | 7869 |
| 529933 | 5904 | 5393 | 5595 | 5677 | 4772 | 4914 | 11918 |

TABLE 15e
in vitro P21 mRNA in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 508031 | 132 | 116 | 119 | 108 | 121 | 185 | 692 |
| 508034 | 119 | 115 | 120 | 117 | 125 | 174 | 344 |
| 508037 | 120 | 119 | 121 | 121 | 117 | 122 | 149 |
| 529933 | 106 | 110 | 101 | 120 | 108 | 108 | 100 |

TABLE 15f
in vitro Gadd45a mRNA in 3T3-L1 cells

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 508031 | 202 | 132 | 198 | 137 | 215 | 570 | 1046 |
| 508034 | 132 | 132 | 167 | 161 | 185 | 475 | 842 |
| 508037 | 175 | 164 | 181 | 175 | 195 | 215 | 416 |
| 529933 | 136 | 136 | 148 | 167 | 169 | 130 | 155 |

For the in vivo dose-response study in the table below, three BALB/C mice per group were administered 3.7, 11.1, 33, or 100 mg/kg 508031 or 3.7, 11.1, 33, 100, or 300 mg/kg 895155 by subcutaneous injection and sacrificed. Levels of Gadd45a, P21, and Tnfrsf1b mRNA were measured by RT-PCR as described in Example 1.

TABLE 15g
Activity and toxicity of modified oligonucleotides
complementary to SOD1

| | position | sugar | |
|---|---|---|---|
| | of altered | modification | |
| Compound | nucleotide in | of altered | ED₅₀ |
| ID | central region | nucleotide | (mg/kg) |
| 508031 | n/a | n/a | 13.12 |
| 895155 | 2 | 2′-OMe | 38.8 |

TABLE 15h
In Vivo Dose-response of Tnfrsf10b mRNA upon treatment
with modified oligonucleotides complementary to SOD1

| Com- | | | | | |
|---|---|---|---|---|---|
| pound | 3.7 mg/kg | 11.1 mg/kg | 33 mg/kg | 100 mg/kg | 300 mg/kg |
| ID | Expression level of Tnfrsf10b mRNA (% Control) | | | | |
| 508031 | 99 | 276 | 3443 | 6446 | n/a |
| 895155 | 81 | 105 | 115 | 193 | 2215 |

TABLE 15i
In Vivo Dose-response of P21 mRNA upon treatment with
modified oligonucleotides complementary to SOD1

| Com- | | | | | |
|---|---|---|---|---|---|
| pound | 3.7 mg/kg | 11.1 mg/kg | 33 mg/kg | 100 mg/kg | 300 mg/kg |
| ID | Expression level of P21 mRNA (% Control) | | | | |
| 508031 | 163 | 222 | 1867 | 3788 | n/a |
| 895155 | 162 | 167 | 167 | 199 | 1467 |

### Example 7 Effect of Position-Specific 2′-Modifications on In Vitro Activity and In Vivo Liver Toxicity of Modified Oligonucleotides Complementary to SRB1

Modified oligonucleotides were synthesized with 2′ modifications as indicated in the table below. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleosides in the central region. Each internucleoside linkage is a phosphorothioate. The compounds in the table below are 100% complementary to the complement of mouse SRB1, GENBANK NT_039313.7 truncated from 566000 to 632000 (SEQ ID NO: 5), at position 64840 to 64855.

TABLE 16
Modified oligonucleotides complementary to SRB1

| | position | | | |
|---|---|---|---|---|
| | of altered | sugar | | |
| | nucleotide | modification | | SEQ |
| Compound | in central | of altered | | ID |
| ID | region | nucleotide | Chemistry Notation (5′-3′) | NO |
| 449093 | n/a | n/a | TₖₛTₖₛ^{m}CₖₛA_{ds}G_{ds}T_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 982030 | 1 | 2′-OMe | TₖₛTₖₛ^{m}CₖₛAₘₛG_{ds}T_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 982031 | 2 | 2′-OMe | TₖₛTₖₛ^{m}CₖₛA_{ds}GₘₛT_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 1042573 | 2 | 2′-MOE | TₖₛTₖₛ^{m}CₖₛA_{ds}GₑₛT_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 1042574 | 2 | cEt | TₖₛTₖₛ^{m}CₖₛA_{ds}GₖₛT_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 1042575 | 2 | 2′-FANA | TₖₛTₖₛ^{m}CₖₛA_{ds}G_{(FANA)s}T_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 1042572 | 2 | 2′-ribo-F | TₖₛTₖₛ^{m}CₖₛA_{ds}G_{fs}T_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 982032 | 3 | 2′-OMe | TₖₛTₖₛ^{m}CₖₛA_{ds}G_{ds}Uₘₛ^{m}C_{ds}A_{ds}T_{ds}G_{ds}A_{ds}^{m}C_{ds}T_{ds}Tₖₛ^{m}Cₖₛ^{m}Cₖ | 29 |
| 1042577 | 3 | 2′-MOE | TₖₛTₖₛ^{m}CₖₛA_{ds}G_{ds}Tₑₛ^{m}C_{ds}A_{ds}T_{ds}G_{ds}A_{ds}^{m}C_{ds}T_{ds}Tₖₛ^{m}Cₖₛ^{m}Cₖ | 28 |
| 1042578 | 3 | cEt | TₖₛTₖₛ^{m}CₖₛA_{ds}G_{ds}Tₖₛ^{m}C_{ds}A_{ds}T_{ds}G_{ds}A_{ds}^{m}C_{ds}T_{ds}Tₖₛ^{m}Cₖₛ^{m}Cₖ | 28 |
| 1042580 | 3 | 2′-FANA | TₖₛTₖₛ^{m}CₖₛA_{ds}G_{ds}U_{(FANA)s}^{m}C_{ds}A_{ds}T_{ds}G_{ds}A_{ds}^{m}C_{ds}T_{ds}Tₖₛ^{m}Cₖₛ^{m}Cₖ | 29 |
| 1042576 | 3 | 2′-ribo-F | TₖₛTₖₛ^{m}CₖₛA_{ds}G_{ds}U_{fs}^{m}C_{ds}A_{ds}T_{ds}G_{ds}A_{ds}^{m}C_{ds}T_{ds}Tₖₛ^{m}Cₖₛ^{m}Cₖ | 29 |

A subscript “d” indicates an unmodified, 2′-β-D-deoxyribosyl sugar moiety.
A subscript “e” indicates a 2′-MOE-β-D-ribofuranosyl sugar moiety.
A subscript “m” indicates a 2′-O-methyl-β-D-ribofuranosyl sugar moiety.
A subscript “k” indicates a cEt.
A subscript “s” indicates a phosphorothioate internucleoside linkage.
A subscript “f” indicates a 2′-F-β-D-2′-deoxyribosyl sugar moiety.
A subscript “(FANA)” indicates a 2′-F-β-D-arabinofuranosyl modified sugar moiety.
A superscript “m” before a C indicates 5-methyl Cytosine.

For in vivo toxicity studies, two BALB/C mice per modified oligonucleotide was administered 150 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

TABLE 17
Toxicity of modified oligonucleotides complementary SRB1

| | position | sugar | |
|---|---|---|---|
| | of altered | modification | ALT @ |
| Compound | nucleotide in | of altered | 150 mg/kg |
| ID | central region | nucleotide | (IU/L) |
| 449093 | n/a | n/a | 2009 |
| 982031 | 2 | 2′-OMe | 2168 |
| 1042573 | 2 | 2′-MOE | 3368 |
| 1042574 | 2 | cEt | 1972 |
| 1042575 | 2 | 2′-FANA | 16335 |
| 1042572 | 2 | 2′-ribo-F | 3563 |
| 982032 | 3 | 2′-OMe | 1630 |
| 1042577 | 3 | 2′-MOE | 2965 |
| 1042578 | 3 | cEt | 3650 |
| 1042580 | 3 | 2′-FANA | 6622 |
| 1042576 | 3 | 2′-ribo-F | 3521 |

For the in vitro study reported in the tables below, 3T3-L1 cells were transfected with 27, 80, 250, 740, 2, 222, 6,667, or 20,000 nM of modified oligonucleotide by electroporation and levels of P21 and Gadd45a were measured by RT-qPCR as described in example 1.

TABLE 17b
in vitro P21 Expression

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 449093 | 99 | 107 | 101 | 104 | 175 | 212 | 255 |
| 982030 | 102 | 100 | 108 | 125 | 172 | 215 | 288 |
| 982031 | 115 | 116 | 114 | 137 | 174 | 204 | 330 |
| 982032 | 107 | 97 | 106 | 112 | 134 | 183 | 224 |

TABLE 17c
in vitro Gadd45a Expression

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 449093 | 124 | 105 | 120 | 105 | 122 | 215 | 350 |
| 982030 | 105 | 103 | 107 | 104 | 126 | 249 | 551 |
| 982031 | 88 | 79 | 86 | 80 | 95 | 182 | 447 |
| 982032 | 82 | 69 | 73 | 76 | 89 | 172 | 366 |

### Example 8 Effect of Inosine Substitution on In Vitro Activity and In Vivo Liver Toxicity of Modified Oligonucleotides Complementary to CXCL12 and Factor XI

TABLE 18
Modified oligonucleotides

| | position of | | | |
|---|---|---|---|---|
| | altered | | | |
| | nucleotide | nucleobase | | SEQ |
| Compound | in central | of altered | | ID |
| ID | region | nucleotide | Chemistry Notation (5′-3′) | NO |
| 1061955 | 2 | Inosine | Gₖₛ^{m}CₖₛAₖₛT_{ds}I_{ds}T_{ds}T_{ds}^{m}C_{ds}T_{ds}^{m}C_{ds}A_{ds}^{m}C_{ds}A_{ds}TₖₛTₖₛAₖ | 30 |
| 1154233 | 2 | Inosine | GₖₛTₖₛ^{m}CₖₛT_{ds}I_{ds}T_{ds}G_{ds}^{m}C_{ds}A_{ds}T_{ds}C_{ds}T_{ds}^{m}C_{ds}Tₖₛ^{m}Cₖₛ^{m}Cₖ | 40 |

A subscript “d” indicates a nucleoside comprising an unmodified, 2′-β-D-deoxyribosyl sugar moiety..
A subscript “k” indicates a cEt. A subscript “s” indicates a phosphorothioate internucleoside linkage.
A superscript “m” before C indicates 5-methyl Cytosine.
I indicates inosine.

For in vitro activity studies, 3T3-L1 cells were plated at 20,000 cells/well and transfected with 27 nM, 80 nM, 250 nM, 740 nM, 2,222 nM, 6,667 nM, or 20,000 nM modified oligonucleotide by electroporation. mRNA was harvested and analyzed by RT-qPCR. CXCL12 mRNA and P21 mRNA were analyzed as in example 1.

Caspase-3 and caspase-7 activation was measured using the Caspase-Glo® 3/7 Assay System (G8090, Promega). Levels of caspase activation correlate with apoptotic cell death. Results are presented relative to the caspase activation in control cells not treated with modified oligonucleotide.

For the in vivo activity and toxicity study in the table below, two BALB/C mice per group were administered 16.7, 50, or 150 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

TABLE 19
Effect of Inosine on activity and toxicity

| | position | | | | | | |
|---|---|---|---|---|---|---|---|
| | of altered | | | in vivo | | | |
| | nucleotide | nucleobase | CXCL12 | CXCL12 | ALT @ | ALT @ | ALT @ |
| Compound | in central | of altered | IC₅₀ | ED₅₀ | 16.7 mg/kg | 50 mg/kg | 150 mg/kg |
| ID | region | nucleotide | (μM) | (mg/kg) | (IU/L) | (IU/L) | (IU/L) |
| 558807* | n/a | n/a | 0.2 | 1.7 | 209 | death | death |
| 1061955 | 2 | Inosine | 0.3 | 4.2 | 20.5 | 26 | 86 |

*Data presented above in Example 4

TABLE 19b
in vitro P21 Expression

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 558807 | 102 | 104 | 105 | 101 | 133 | 191 | 301 |
| 1061955 | 117 | 116 | 106 | 104 | 104 | 121 | 149 |

TABLE 19c
in vitro Caspase Activation

| | | | | | 2,222 | 6,667 | 20,000 |
|---|---|---|---|---|---|---|---|
| Compound | 27 nM | 80 nM | 250 nM | 740 nM | nM | nM | nM |
| ID | Relative Caspase Activation (% Control) | | | | | | |
| 558807 | 135 | 110 | 131 | 115 | 147 | 476 | 462 |
| 1061955 | 75 | 81 | 134 | 120 | 121 | 162 | 170 |

For the study in the tables below, two BALB/C mice per group were administered 1.8 mg/kg, 5.5 mg/kg, 16.7 mg/kg, 50 mg/kg, or 150 mg/kg modified oligonucleotide by subcutaneous injection and sacrificed after 72 hours. 558807 was administered at 1.8 mg/kg, 5.5 mg/kg, 16.7 mg/kg due to acute toxicity at higher doses. Expression levels of Gadd45a, Tnfrsf10b, and P21 mRNA were measured as described in Example 1. Data for 558807 was also presented in Example 2, Tables 6b-6d.

TABLE 19d
In Vivo Dose-response of Gadd45a mRNA upon treatment with
modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of Gadd45a mRNA (% Control) | | | | |
| 558807 | 122 | 211 | 278 | n/a | n/a |
| 1061955 | 109 | 86 | 93 | 84 | 123 |

TABLE 19e
In Vivo Dose-response of Tnfrsf10b mRNA upon treatment
with modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of Tnfrsf10b mRNA (% Control) | | | | |
| 558807 | 160 | 166 | 271 | n/a | n/a |
| 1061955 | 158 | 77 | 126 | 134 | 192 |

TABLE 19f
In Vivo Dose-response of P21 mRNA upon treatment with
modified oligonucleotides complementary to CXCL12

| Com- | | | | | |
|---|---|---|---|---|---|
| pound | 1.8 mg/kg | 5.5 mg/kg | 16.7 mg/kg | 50 mg/kg | 150 mg/kg |
| ID | Expression level of P21 mRNA (% Control) | | | | |
| 558807 | 173 | 148 | 1456 | n/a | n/a |
| 1061955 | 36 | 8.6 | 16 | 33 | 72 |

For the in vivo activity and toxicity study in the table below, two BALB/C mice per group were administered 33 or 100 mg/kg modified oligonucleotide by subcutaneous injection and sacrificed 72 hours later. Four mice were administered an injection of saline as a control. FXI mRNA expression was measured by RT-qPCR as described in Example 3. Plasma levels of ALT were measured using an automated clinical chemistry analyzer. Increased ALT is indicative of acute liver toxicity.

TABLE 19g
Effect of Inosine on activity and toxicity

| | position | | | | | |
|---|---|---|---|---|---|---|
| | of altered | | | | | |
| | nucleotide | nucleobase | FXI mRNA | FXI mRNA | ALT @ | ALT @ |
| Compound | in central | of altered | @ 33 mg/kg | @ 100 mg/kg | 33 mg/kg | 100 mg/kg |
| ID | region | nucleotide | (% control) | (% control) | (IU/L) | (IU/L) |
| 464917* | n/a | n/a | 10.9 | 0.9 | 7511 | 31066 |
| 1154233 | 2 | Inosine | 5.0 | 1.2 | 315 | 4553 |

*Data presented above in Example 4

### Example 9 Effect of Position-Specific Nucleobase Substitutions on In Vitro Activity and In Vivo Liver Toxicity of Modified Oligonucleotides Complementary to CXCL12

Modified oligonucleotides containing nucleobase modifications at various positions were synthesized. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleobases in the central region. The compounds in the table below are 100% complementary to mouse CXCL12, GENBANK NT_039353.7 truncated from 69430515 to 69445350 (SEQ ID NO: 1), at position 6877 to 6892.

Nucleobase modifications:

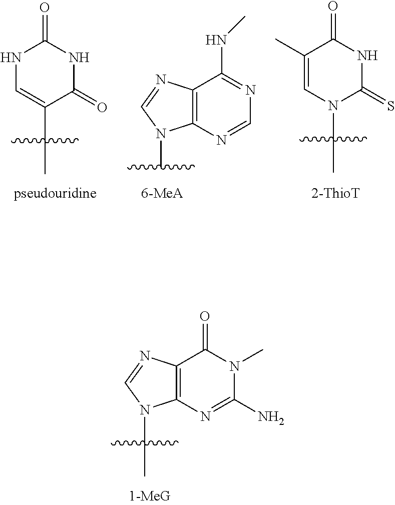

TABLE 20
Modified oligonucleotides

| | position of | | | |
|---|---|---|---|---|
| | altered | | | |
| | nucleotide | | | SEQ |
| Compound | in central | nucleobase of | | ID |
| ID | region | altered nucleotide | Chemistry Notation (5′-3′) | NO |
| 1076587 | 2 | 6-MeA, 2′-OH | Gₖₛ^{m}CₖₛAₖₛT_{ds}^{m6}AᵣₛT_{ds}T_{ds}^{m}C_{ds}T_{ds}^{m}C_{ds}A_{ds}^{m}C_{ds}A_{ds}TₖₛTₖₛAₖ | 31 |
| 1076588 | 3 | 6-MeA, 2′-OH | Gₖₛ^{m}CₖₛAₖₛT_{ds}G_{ds}^{m6}AᵣₛT_{ds}^{m}C_{ds}T_{ds}^{m}C_{ds}A_{ds}^{m}C_{ds}A_{ds}TₖₛTₖₛAₖ | 32 |
| 1069852 | 2 | pseudouridine, 2′-OH | Gₖₛ^{m}CₖₛAₖₛT_{ds}ΨᵣₛT_{ds}T_{ds}^{m}C_{ds}T_{ds}^{m}C_{ds}A_{ds}^{m}C_{ds}A_{ds}TₖₛTₖₛAₖ | 33 |
| 1061328 | 3 | pseudouridine, 2′-OH | Gₖₛ^{m}CₖₛAₖₛT_{ds}G_{ds}ΨᵣₛT_{ds}^{m}C_{ds}T_{ds}^{m}C_{ds}A_{ds}^{m}C_{ds}A_{ds}TₖₛTₖₛAₖ | 34 |
| 1016673 | 1 | pseudouridine, 2′-H | Gₖₛ^{m}CₖₛAₖₛT_{ds}Ψ_{ds}G_{ds}T_{ds}^{m}C_{ds}T_{ds}^{m}C_{ds}A_{ds}^{m}C_{ds}A_{ds}TₖₛTₖₛAₖ | 35 |
| 1004684 | 3 | pseudouridine, 2′-H | Gₖₛ^{m}CₖₛAₖₛT_{ds}T_{ds}Ψ_{ds}T_{ds}^{m}C_{ds}T_{ds}^{m}C_{ds}A_{ds}^{m}C_{ds}A_{ds}TₖₛTₖₛAₖ | 36 |
| 1004685 | 4 | pseudouridine, 2′-H | Gₖₛ^{m}CₖₛAₖₛT_{ds}G_{ds}T_{ds}Ψ_{ds}^{m}C_{ds}T_{ds}^{m}C_{ds}A_{ds}^{m}C_{ds}A_{ds}TₖₛTₖₛAₖ | 37 |
| 1016674 | 6 | pseudouridine, 2′-H | Gₖₛ^{m}CₖₛAₖₛT_{ds}G_{ds}T_{ds}^{m}C_{ds}T_{ds}Ψ_{ds}^{m}C_{ds}A_{ds}^{m}C_{ds}A_{ds}TₖₛTₖₛAₖ | 38 |
| 863089 | 1 | 2-thioT | Gₖₛ^{m}CₖₛAₖₛ^{s}T_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 863090 | 3 | 2-thioT | Gₖₛ^{m}CₖₛAₖₛT_{ds}G_{ds}^{s}T_{ds}T_{ds}^{m}C_{ds}T_{ds}^{m}C_{ds}A_{ds}^{m}C_{ds}A_{ds}TₖₛTₖₛAₖ | 18 |
| 863091 | 4 | 2-thioT | Gₖₛ^{m}CₖₛAₖₛT_{ds}G_{ds}T_{ds}^{s}T_{ds}^{m}C_{ds}T_{ds}^{m}C_{ds}A_{ds}^{m}C_{ds}A_{ds}TₖₛTₖₛAₖ | 18 |
| 863092 | 6 | 2-thioT | Gₖₛ^{m}CₖₛAₖₛT_{ds}G_{ds}T_{ds}T_{ds}^{m}C_{ds}^{s}T_{ds}^{m}C_{ds}A_{ds}^{m}C_{ds}A_{ds}TₖₛTₖₛAₖ | 18 |
| 1061964 | 2 | 1-MeG, 2′-OH | Gₖₛ^{m}CₖₛAₖₛT_{ds}^{ml}GᵣₛT_{ds}T_{ds}^{m}C_{ds}T_{ds}^{m}C_{ds}A_{ds}^{m}C_{ds}A_{ds}TₖₛTₖₛAₖ | 18 |

A subscript “d” indicates an unmodified, 2′-β-D-deoxyribosyl sugar moiety.
A subscript “r” indicates a unmodified, β-D-ribofuranosyl sugar moiety.
A subscript “k” indicates a cEt.
A subscript “s” indicates a phosphorothioate internucleoside linkage.
A superscript “m” before C indicates 5-methyl Cytosine.
A subscript “s” indicates a phosphorothioate internucleoside linkage.
A superscript “m” before a nucleobase indicator indicates that the nucleobase has a 5-methyl group, such as methyl Cytosine, methyl Adenosine, or methyl Guanosine.
A superscript “m6” before a A indicates 6-methyl Adenosine Ψ represents the nucleobase pseudouridine.
^{S}T represents the nucleobase 2-thiothymidine.

For in vitro activity studies, 3T3-L1 cells were plated at 20,000 cells/well and transfected with 10 nM, 30 nM, 250 nM, 740 nM, 2,220 nM, 6, 667 nM, or 20,000 nM modified oligonucleotide by electroporation. mRNA was harvested and analyzed by RT-qPCR. CXCL12 and RAPTOR mRNA was analyzed as in Example 1. The in vitro caspase assay was performed as described in Example 4.

For the in vivo toxicity study in the table below, two BALB/C mice per modified oligonucleotide were administered 50 or150 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

TABLE 21
Effect of modified nucleobases on activity and toxicity

| | position | | | | | |
|---|---|---|---|---|---|---|
| | of altered | | | | | |
| | nucleotide | | | | ALT @ | ALT @ |
| Compound | in central | nucleobase of | CXCL12 | RAPTOR | 50 mg/kg | 150 mg/kg |
| ID | region | altered nucleotide | IC₅₀ (nM) | IC₅₀ (nM) | (IU/L) | (IU/L) |
| 558807 | n/a | n/a | 47 | 800 | 6303 | death |
| 1076587 | 2 | 6-MeA | 300 | 18 | n.d. | n.d. |
| 1076588 | 3 | 6-MeA | 1400 | 13 | n.d. | n.d. |
| 1016673 | 1 | pseudouridine, 2′-H | 156 | 3600 | n.d. | n.d. |
| 1004684 | 2 | pseudouridine, 2′-H | 105 | 2600 | n.d. | n.d. |
| 1004685 | 3 | pseudouridine, 2′-H | 157 | 4100 | n.d. | n.d. |
| 1016674 | 4 | pseudouridine, 2′-H | 142 | 3800 | n.d. | n.d. |
| 863089 | 1 | 2-thioT | 48 | 8800 | 390 | 3620 |
| 863090 | 3 | 2-thioT | 130 | 1400 | death | death |
| 863091 | 4 | 2-thioT | 155 | 1700 | 6237 | death |
| 863092 | 6 | 2-thioT | 110 | 1900 | 14514.5 | death |
| 1061964 | 2 | 1-MeG | 5200 | 8600 | n.d. | n.d. |

TABLE 21b
in vitro Caspase Activation

| | | | | | 2222 | 6667 | 20,000 |
|---|---|---|---|---|---|---|---|
| Compound | 27 nM | 80 nM | 250 nM | 740 nM | nM | nM | nM |
| ID | Relative Caspase Activation (% Control) | | | | | | |
| 558807 | 100 | 100 | 100 | 97 | 110 | 202 | 298 |
| 1076587 | 90 | 86 | 80 | 82 | 81 | 77 | 94 |
| 1076588 | 91 | 91 | 96 | 91 | 96 | 97 | 114 |
| 1069852 | 97 | 87 | 105 | 100 | 89 | 79 | 85 |
| 1061328 | 92 | 95 | 96 | 98 | 102 | 153 | 199 |

TABLE 21c
in vitro Caspase Activation

| | | | | | 2,222 | 6,667 | 20,000 |
|---|---|---|---|---|---|---|---|
| Compound | 27 nM | 80 nM | 250 nM | 740 nM | nM | nM | nM |
| ID | Relative Caspase Activation (% Control) | | | | | | |
| 558807 | 135 | 110 | 131 | 115 | 147 | 476 | 462 |
| 1061964 | 107 | 142 | 140 | 149 | 135 | 123 | 125 |

TABLE 21d
in vitro P21 Expression

| | | | | | 2,222 | 6,667 | 20,000 |
|---|---|---|---|---|---|---|---|
| Compound | 27 nM | 80 nM | 250 nM | 740 nM | nM | nM | nM |
| ID | Expression level of P21 mRNA (% Control) | | | | | | |
| 558807 | 102 | 104 | 105 | 101 | 133 | 191 | 301 |
| 1061964 | 121 | 110 | 115 | 90 | 107 | 102 | 90 |

For in vivo activity and toxicity study in the table below, 2 BALB/C mice per group were administered modified oligonucleotide by subcutaneous injection and sacrificed after 72 hours. Compounds were dosed at 0.6, 1.8, 5.5, 16.7, 50 or 150 mg/kg. Tissue were collected and mRNA was isolated and levels of CXCL12 were measured by RT-qPCR with primer probe set RTS2605 as described above. Levels of Gadd45a were analyzed using primer probe set Mm00432802_m1 (ThermoFisher). Levels of P21 were analyzed using primer probe set Mm04207341_m1 (ThermoFisher). Levels of Tnfrsf10b were analyzed using primer probe set Mm00457866_m1 (ThermoFisher). Expression levels were normalized with Ribogreen® and are presented relative to levels in mice treated with PBS.

TABLE 21e
In Vivo of modified oligonucleotides complementary
to CXCL12 containing 2-Thio-T

| | 2-Thio-T | | | |
|---|---|---|---|---|
| | position | ALT@16.7 | ALT @50 | ALT @ 150 |
| Compound | in central | mg/kg | mg/kg | mg/kg |
| ID | region | (IU/L) | (IU/L) | (IU/L) |
| PBS | n/a | 27 (@0 mg/kg) | | |
| 558807 | n/a | 2002 | 6303 | death |
| 863089 | 1 | 60 | 390 | 3620 |
| 863090 | 3 | 4929 | death | death |
| 863091 | 4 | 1894 | 6237 | death |
| 863092 | 6 | 1073 | 14515 | death |

TABLE 21f
In Vivo Activity of modified oligonucleotides
complementary to CXCL12 containing 2-Thio-T

| | 0.6 | 1.8 | 5.5 | 16.7 | 50 | 150 |
|---|---|---|---|---|---|---|
| Compound | mg/kg | mg/kg | mg/kg | mg/kg | mg/kg | mg/kg |
| ID | Expression level of CXCL12 mRNA (% Control) | | | | | |
| 558807 | 65 | 34 | 14 | 4 | 7 | n.d. |
| 863089 | 72 | 51 | 33 | 16 | 14 | 8 |
| 863090 | 58 | 31 | 11 | 11 | 0 | 0 |
| 863091 | 66 | 28 | 24 | 12 | 12 | 0 |
| 863092 | 59 | 42 | 20 | 5 | 6 | 0 |

### Example 10 Effect of Position-Specific Morpholinos on In Vitro Activity and In Vivo Liver Toxicity of Modified Oligonucleotides Complementary to CXCL12

Modified oligonucleotides containing morpholinos at various positions were synthesized. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleobases in the central region. The compounds in the table below are 10000 complementary to mouse CXCL12, GENBANK NT_039353.7 truncated from 69430515 to 69445350 (SEQ ID NO: 1), at position 6877 to 6892. Morpholino residues replace a full nucleotide, including the internucleoside linkage, and have the structures shown below.

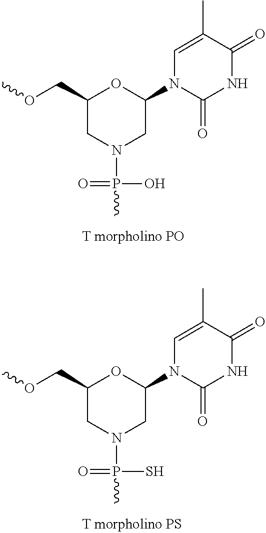

TABLE 22
Modified oligonucleotides

| | morpholino | | | |
|---|---|---|---|---|
| | position in | | | SEQ |
| Compound | central | morpholino | | ID |
| ID | region | type | Chemistry Notation (5′-3′) | NO |
| 1044689 | 1 | T-PO | Gₖₛ^{m}CₖₛAₖₛ(MPₒ^{T})G_{ds}T_{ds}T_{ds}^{m}C_{ds}T_{ds}^{m}C_{ds}A_{ds}^{m}C_{ds}A_{ds}TₖₛTₖₛAₖ | 18 |
| 1044690 | 3 | T-PO | Gₖₛ^{m}CₖₛAₖₛT_{ds}G_{ds}(MPₒ^{T})T_{ds}^{m}C_{ds}T_{ds}^{m}C_{ds}A_{ds}^{m}C_{ds}A_{ds}TₖₛTₖₛAₖ | 18 |
| 1044691 | 4 | T-PO | Gₖₛ^{m}CₖₛAₖₛT_{ds}G_{ds}T_{ds}(MPₒ^{T})^{m}C_{ds}T_{ds}^{m}C_{ds}A_{ds}^{m}C_{ds}A_{ds}TₖₛTₖₛAₖ | 18 |
| 1044692 | 6 | T-PO | Gₖₛ^{m}CₖₛAₖₛT_{ds}G_{ds}T_{ds}T_{ds}^{m}C_{ds}(MPₒ^{T})^{m}C_{ds}A_{ds}^{m}C_{ds}A_{ds}TₖₛTₖₛAₖ | 18 |
| 1048416 | 1 | T-PS | Gₖₛ^{m}CₖₛAₖₛ(MPₒ^{T})G_{ds}T_{ds}T_{ds}^{m}C_{ds}T_{ds}^{m}C_{ds}A_{ds}^{m}C_{ds}A_{ds}TₖₛTₖₛAₖ | 18 |
| 1048417 | 3 | T-PS | Gₖₛ^{m}CₖₛAₖₛT_{ds}G_{ds}(MPₛ^{T})T_{ds}^{m}C_{ds}T_{ds}^{m}C_{ds}A_{ds}^{m}C_{ds}A_{ds}TₖₛTₖₛAₖ | 18 |
| 1048418 | 4 | T-PS | Gₖₛ^{m}CₖₛAₖₛT_{ds}G_{ds}T_{ds}(MPₛ^{T})^{m}C_{ds}T_{ds}^{m}C_{ds}A_{ds}^{m}C_{ds}A_{ds}TₖₛTₖₛAₖ | 18 |
| 1048419 | 6 | T-PS | Gₖₛ^{m}CₖₛAₖₛT_{ds}G_{ds}T_{ds}T_{ds}^{m}C_{ds}(MPₛ^{T})^{m}C_{ds}A_{ds}^{m}C_{ds}A_{ds}TₖₛTₖₛAₖ | 18 |

A subscript “d” indicates a nucleoside comprising an unmodified, 2′-β-D-deoxyribosyl sugar moiety.
A subscript “r” indicates a nucleoside comprising an unmodified, β-D-ribofuranosyl sugar moiety.
A subscript “k” indicates a cEt.
A subscript “s” indicates a phosphorothioate internucleoside linkage.
A superscript “m” before C indicates 5-methyl Cytosine.
A (MP₀^{T}) represents a phosphate thymidine morpholino, while (MPₛ^{T}) represents a phosphorothioate thymidine morpholino.

TABLE 22
Effect of morpholinos on in vitro activity and toxicity

| | morpholino | | | |
|---|---|---|---|---|
| | position | | CXCL12 | RAPTOR |
| Compound | in central | morpholino | IC₅₀ | IC₅₀ |
| ID | region | type | (nM) | (nM) |
| 558807 | n/a | n/a | 47 | 800 |
| 1044689 | 1 | T-PO | 405 | >20000 |
| 1044690 | 3 | T-PO | 182 | 4100 |
| 1044691 | 4 | T-PO | 128 | 4400 |
| 1044692 | 6 | T-PO | 145 | 1900 |
| 1048416 | 1 | T-PS | 333 | >20000 |
| 1048417 | 3 | T-PS | 159 | 3300 |
| 1048418 | 4 | T-PS | 134 | 5200 |
| 1048419 | 6 | T-PS | 119 | 1100 |

### Example 11 Effect of Position-Specific MOP on In Vitro Activity and In Vivo Toxicity of Modified Oligonucleotides Complementary CXCL12, Factor XI, PTEN, and SOD-1

Modified oligonucleotides were synthesized with MOP neutral backbone linkages at specific positions in place of phosphorothioate linkages. These modified oligonucleotides were compared to the otherwise identical modified oligonucleotide lacking the altered nucleotide in the central region, a 3-10-3 cEt modified oligonucleotide, containing three cEt nucleosides in each of the 5′ and 3′ regions and 10 unmodified DNA nucleosides in the central region, and phosphorothioate linkages throughout. The compounds in the table below are 100% complementary to mouse CXCL12, Factor XI, PTEN, or SOD-1, with sequences described above.

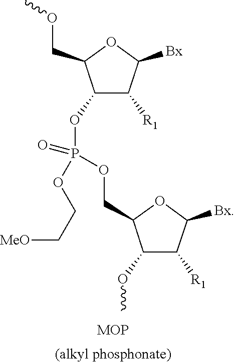

TABLE 23
Modified oligonucleotides containing MOP linkages

| | MOP | | | |
|---|---|---|---|---|
| | position | | | |
| | in | | | SEQ |
| Compound | central | | | ID |
| ID | region | Target | Chemistry Notation (5′-3′) | NO |
| 766676 | 1 | CXCL12 | Gₖₛ^{m}CₖₛAₖₛT_{dx}G_{ds}T_{ds}T_{ds}^{m}C_{ds}T_{ds}^{m}C_{ds}A_{ds}^{m}C_{ds}A_{ds}TₖₛTₖₛAₖ | 18 |
| 766677 | 2 | CXCL12 | Gₖₛ^{m}CₖₛAₖₛT_{ds}G_{dx}T_{ds}T_{ds}^{m}C_{ds}T_{ds}^{m}C_{ds}A_{ds}^{m}C_{ds}A_{ds}TₖₛTₖₛAₖ | 18 |
| 766678 | 3 | CXCL12 | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ | 18 |
| 766679 | 4 | CXCL12 | Gₖₛ^{m}CₖₛAₖₛT_{ds}G_{ds}T_{ds}T_{dx}^{m}C_{ds}T_{ds}^{m}C_{ds}A_{ds}^{m}C_{ds}A_{ds}TₖₛTₖₛAₖ | 18 |
| 766680 | 5 | CXCL12 | Gₖₛ^{m}CₖₛAₖₛT_{ds}G_{ds}T_{ds}T_{ds}^{m}C_{dx}T_{ds}^{m}C_{ds}A_{ds}^{m}C_{ds}A_{ds}TₖₛTₖₛAₖ | 18 |
| 766681 | 6 | CXCL12 | Gₖₛ^{m}CₖₛAₖₛT_{ds}G_{ds}T_{ds}T_{ds}^{m}C_{ds}T_{dx}^{m}C_{ds}A_{ds}^{m}C_{ds}A_{ds}TₖₛTₖₛAₖ | 18 |
| 766682 | 7 | CXCL12 | Gₖₛ^{m}CₖₛAₖₛT_{ds}G_{ds}T_{ds}T_{ds}^{m}C_{ds}T_{ds}^{m}C_{dx}A_{ds}^{m}C_{ds}A_{ds}TₖₛTₖₛAₖ | 18 |
| 766683 | 8 | CXCL12 | Gₖₛ^{m}CₖₛAₖₛT_{ds}G_{ds}T_{ds}T_{ds}^{m}C_{ds}T_{ds}^{m}C_{ds}A_{dx}^{m}C_{ds}A_{ds}TₖₛTₖₛAₖ | 18 |
| 766684 | 9 | CXCL12 | Gₖₛ^{m}CₖₛAₖₛT_{ds}G_{ds}T_{ds}T_{ds}^{m}C_{ds}T_{ds}^{m}C_{ds}A_{ds}^{m}C_{dx}A_{ds}TₖₛTₖₛAₖ | 18 |
| 766685 | 10 | CXCL12 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{dx}TₖₛTₖₛAₖ | 18 |
| 965605 | 2 | FactorXI | GₖₛTₖₛ^{m}CₖₛT_{ds}G_{dx}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 965606 | 3 | FactorXI | GₖₛTₖₛ^{m}CₖₛT_{ds}G_{ds}T_{dx}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 985299 | 2 | PTEN | AₖₛTₖₛ^{m}CₖₛA_{ds}T_{dx}G_{ds}G_{ds}^{m}C_{ds}T_{ds}G_{ds}^{m}C_{ds}A_{ds}G_{ds}^{m}CₖₛTₖₛTₖ | 24 |
| 985303 | 3 | PTEN | AₖₛTₖₛ^{m}CₖₛA_{ds}T_{ds}G_{dx}G_{ds}^{m}C_{ds}T_{ds}G_{ds}^{m}C_{ds}A_{ds}G_{ds}^{m}CₖₛTₖₛTₖ | 24 |
| 985307 | 2 | SOD1 | TₖₛGₖₛAₖₛG_{ds}G_{dx}T_{ds}^{m}C_{ds}^{m}C_{ds}T_{ds}G_{ds}^{m}C_{ds}A_{ds}^{m}C_{ds}TₖₛGₖₛGₖ | 26 |
| 985311 | 3 | SOD1 | TₖₛGₖₛAₖₛG_{ds}G_{ds}T_{dx}^{m}C_{ds}^{m}C_{ds}T_{ds}G_{ds}^{m}C_{ds}A_{ds}^{m}C_{ds}TₖₛGₖₛGₖ | 26 |

A subscript “d” indicates an unmodified, 2′-β-D-deoxyribosyl sugar moiety.
A subscript “k” indicates a cEt.
A subscript “s” indicates a phosphorothioate internucleoside linkage.
A superscript “m” before a C indicates 5-methyl Cytosine.
A subscript “x” indicates a MOP or methoxypropyl internucleoside linkage.
The postion of the internucleoside linkage is designated as the position of the nucleoside that is on the 5’ end of the linkage.

For in vitro activity studies for compounds complementary to CXCL12, b.END cells were plated at 20,000 cells/well and transfected with 0, 27 nM, 82 nM, 247 nM, 740 nM, 2,220 nM, 6,660 nM or 20,000 nM antisense oligonucleotide by electroporation. mRNA was harvested and analyzed by RT-qPCR. CXCL12 and raptor mRNA was analyzed as in previous examples.

For in vitro activity studies for compounds complementary to Factor XI, PTEN or SOD1, 3T3-L1 cells were plated at 20,000 cells/well and transfected with 0, 27 nM, 82 nM, 247 nM, 740 nM, 2,220 nM, 6,660 nM or 20,000 nM antisense oligonucleotide by electroporation. mRNA was harvested and analyzed by RT-qPCR. Complementary mRNA and raptor mRNA was analyzed as in previous examples.

For the in vivo toxicity study in the table below, one or two BALB/C mice per modified oligonucleotide were administered modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

For the in vivo activity study in the table below, two to four BALB/C mice per dosing group were administered modified oligonucleotide by subcutaneous injection and sacrificed 24 hours later. Mice were administered 1.9 mg/kg, 5.6 mg/kg, 16.7 mg/kg, 50 mg/k or 150 mg/kg of compound 558807 or 766676-766685. Mice were administered 6.25 mg/kg, 12.5 mg/kg, 25 mg/kg, 50 mg/kg, or 100 mg/kg modified oligonucleotide for compounds 965605 and 965606. Mice were administered 6.25 mg/kg, 12.5 mg/kg, 25 mg/kg, or 100 mg/kg modified oligonucleotide for 464917. Mice were administered 6.25 mg/kg, 12.5 mg/kg, 25 mg/kg, or 150 mg/kg modified oligonucleotide for 482050. Mice were administered 6.25 mg/kg, 12.5 mg/kg, 25 mg/kg, 50 mg/kg or 150 mg/kg modified oligonucleotide for 985299 and 985303. Mice were administered 12.5 mg/kg, 25 mg/kg, 50 mg/kg or 150 mg/kg modified oligonucleotide for 508031, 985307, and 985311. Two animals were administered an injection of saline as a control.

TABLE 24
Effect of MOP backbone modifications on activity and toxicity

| | MOP | | | | in vivo | |
|---|---|---|---|---|---|---|
| | position | | Complementary | RAPTOR | Complementary | ALT @ |
| Compound | in central | Complementary | mRNA IC₅₀ | IC₅₀ | mRNA ED₅₀ | Max dose* |
| ID | region | mRNA | (μM) | (μM) | (mg/kg) | (IU/L) |
| PBS | n/a | n/a | n/a | n/a | n/a | 23 |
| 558807 | n/a | CXCL12 | 0.095 | 1.26 | 2.9 | death |
| 766676 | 1 | CXCL12 | 0.100 | 5.8 | 4.5 | 7764 |
| 766677 | 2 | CXCL12 | 0.110 | >>10 | 6.8 | 46 |
| 766678 | 3 | CXCL12 | 0.115 | >>10 | 6.2 | 44 |
| 766679 | 4 | CXCL12 | 0.080 | 7.2 | 5.7 | 4481 |
| 766680 | 5 | CXCL12 | 0.085 | 3.5 | 5.1 | 9139 |
| 766681 | 6 | CXCL12 | 0.080 | 3.0 | 3.6 | 17846 |
| 766682 | 7 | CXCL12 | 0.090 | 3.8 | 4.4 | 12510 |
| 766683 | 8 | CXCL12 | 0.070 | 2.2 | 4.3 | death |
| 766684 | 9 | CXCL12 | 0.090 | 2.2 | 3.1 | death |
| 766685 | 10 | CXCL12 | 0.090 | 2.0 | 2.1 | death |
| 464917 | n/a | Factor XI | n.d. | 1.6 | 6.9 | 33848 |
| 965605 | 2 | Factor XI | n.d. | 3.7 | 10.2 | 3464 |
| 965606 | 3 | Factor XI | n.d. | 7.7 | 12.3 | 1160 |
| 482050 | n/a | PTEN | 3.9 | 2.4 | 67 | 2458 |
| 985299 | 2 | PTEN | 1.1 | 4 | 120 | 767 |
| 985303 | 3 | PTEN | 1.7 | 3.7 | 194 | 43 |
| 508031 | n/a | SOD1 | 0.03 | 0.46 | 63 | 21934 |
| 985307 | 2 | SOD1 | 0.17 | 3.6 | 157 | 57 |
| 985311 | 3 | SOD1 | 0.78 | 5.1 | 173 | 71 |

*ALT at 150 mg/kg for CXCL12 oligonucleotides, 100 mg/kg for Factor XI oligonucleotides, 200 mg/kg for PTEN oligonucleotides 985299 and 985303, 100 mg/kg for PTEN oligonucleotide 482050 and 150 mg/kg for SOD1 oligonucleotides 985307 and 985311, and 100 mg/kg for SOD1 oligonucleotide 508031.
**Value represents the average of two independent experiments

Relative caspase activation in 3T3-L1 cells was determined as described in Example 4.

For the in vitro study reported in the tables below, b.END cells were electroporated with 27 nM, 80 nM, 250 nM, 740 nM, 2, 222 nM, 6,667 nM, or 20,000 nM of modified oligonucleotide and levels of P21 were measured by RT-qPCR using primer probe set Mm04207341_m1 (ThermoFisher).

Selected modified nucleotides described in above were tested for their effect on HeLa cells by microscopy. HeLa cells were transfected by lipofectamine 2000 with 200 nM of modified oligonucleotide for 2 hrs and then cellular protein p54nrb was stained by mP54 antibody (Santa Cruz Biotech, sc-376865) and DAPI was used to stain for the nucleus of cells. The number of cells with nucleolar p54nrb and the total number of cells in the images were counted.

TABLE 24a
Effect of MOP backbone modifications on activity and toxicity

| | MOP | | | | |
|---|---|---|---|---|---|
| Com- | position | Comple- | | in vitro | % |
| pound | in central | mentary | Caspase | p21 mRNA | nucleolar |
| ID | region | mRNA | (% mock) | (% saline) | p54nrb |
| 558807 | n/a | CXCL12 | 313 | 243 | 82 |
| 766676 | 1 | CXCL12 | 243 | 187 | 32 |
| 766677 | 2 | CXCL12 | 121 | 179 | 25 |
| 766678 | 3 | CXCL12 | 136 | 180 | 32 |
| 766679 | 4 | CXCL12 | 240 | 195 | 39 |
| 766680 | 5 | CXCL12 | 351 | 263 | 86 |
| 766681 | 6 | CXCL12 | 315 | 309 | 79 |
| 766682 | 7 | CXCL12 | 345 | 236 | 71 |
| 766683 | 8 | CXCL12 | 257 | 260 | 91 |
| 766684 | 9 | CXCL12 | 314 | 247 | 88 |
| 766685 | 10 | CXCL12 | 308 | 291 | 90 |

For the in vivo toxicity study in the table below, two BALB/C mice per dosing group were administered modified oligonucleotide by subcutaneous injection and sacrificed 72 hours later. Mice were administered 10 mg/kg, 33 mg/kg, or 100 mg/kg modified oligonucleotide for compounds 464917, 965605, and 965606 and 10 mg/kg, 100 mg/kg, or 200 mg/kg for 482050, 985299, and 985303. Two animals were administered an injection of saline as a control. Plasma levels of ALT were measured using an automated clinical chemistry analyzer. Increased ALT is indicative of acute liver toxicity. Therapeutic index was calculated as the ratio of maximum non-toxic dose (MNTD)/ED₅₀, where ED₅₀ was determined via the in vivo activity study reported in the table above.

TABLE 24b
in vivo Toxicity improvement for modified
oligonucleotides complementary to Factor XI

| Compound | ALT @ | ALT @ | ALT @ | TI |
|---|---|---|---|---|
| ID | 10 mg/kg | 33 mg/kg | 100 mg/kg | (MNTD/ED50) |
| 464917 | 239 | 8199 | 33848 | 1.4 |
| 965605 | 46 | 125 | 3464 | 3.2 |
| 965606 | 55 | 77 | 1160 | 2.7 |

TABLE 24c
in vivo Toxicity improvement for modified
oligonucleotides complementary to PTEN

| Compound | ALT @ | ALT @ | ALT @ | TI |
|---|---|---|---|---|
| ID | 10 mg/kg | 100 mg/kg | 200 mg/kg | (MNTD/ED50) |
| 482050 | 55 | 9496 | 5329 | 0.15 |
| 985299 | 45 | 164 | 767 | 0.8 |
| 985303 | 33 | 39 | 43 | 1.0 |

TABLE 24d
in vivo Toxicity improvement for modified
oligonucleotides complementary to SOD1

| Compound | ALT @ | ALT @ | TI |
|---|---|---|---|
| ID | 50 mg/kg | 150 mg/kg | (MNTD/ED50) |
| 482050 | 2189 | n.d. | <0.8 |
| 985307 | n.d. | 57 | >1.0 |
| 985311 | n.d. | 71 | >0.9 |

### Example 12 Effect of Position-Specific MOP in Combination with 2′-Modifications

Modified oligonucleotides were synthesized with MOP neutral backbone linkages at specific positions in place of phosphorothioate linkages in combination with 2′-FANA or 2′-OMe modified sugar moieties. These modified oligonucleotides were compared to the otherwise identical modified oligonucleotide lacking a modified nucleoside in the central region, a 3-10-3 cEt modified oligonucleotide, containing three cEt nucleosides in each of the 5′ and 3′ regions and 10 unmodified DNA nucleosides in the central region, and phosphorothioate linkages throughout. The compounds in the table below are 100% complementary to mouse CXCL12 or SOD1, with sequences as described above.

3′-HPPO-GalNAc refers to the structure below, wherein the phosphate group is attached to the 3′-oxygen atom of the 3′ nucleoside:

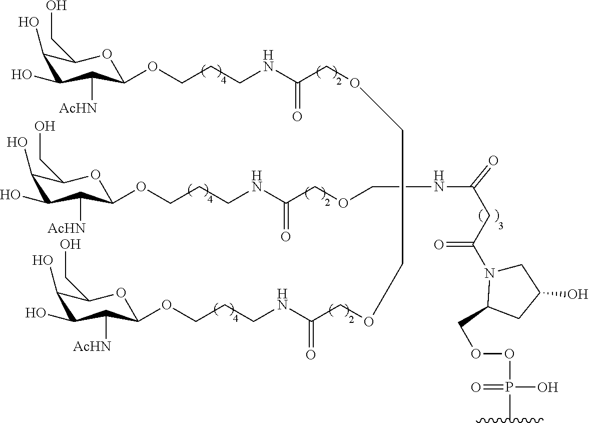

TABLE 25
Modified oligonucleotides containing MOP linkages and 2′-Modifications

| | | 2′-altered | sugar | | | |
|---|---|---|---|---|---|---|
| | MOP | nucleotid | modification | | | |
| | position | eposition | of 2′- | | | SEQ |
| Compound | in central | in central | altered | | | ID |
| ID | region | region | nucleotide | Target | Chemistry Notation (5′-3′) | NO |
| 1061302 | 1 | 1 | 2′-OMe | CXCL12 | Gₖₛ^{m}CₖₛAₖₛTₘₓG_{ds}T_{ds}T_{ds}^{m}C_{ds}T_{ds}^{m}C_{ds}A_{ds} | 18 |
| | | | | | ^{m}C_{ds}A_{ds}TₖₛTₖₛAₖ | |
| 1061303 | 2 | 2 | 2′-OMe | CXCL12 | Gₖₛ^{m}CₖₛAₖₛT_{ds}GₘₓT_{ds}T_{ds}^{m}C_{ds}T_{ds}^{m}C_{ds}A_{ds} | 18 |
| | | | | | ^{m}C_{ds}A_{ds}T_{kS}TₖₛAₖ | |
| 1061304 | 3 | 3 | 2′-OMe | CXCL12 | Gₖₛ^{m}CₖₛAₖₛT_{ds}G_{ds}TₘₓT_{ds}^{m}C_{ds}T_{ds}^{m}C_{ds}A_{ds} | 18 |
| | | | | | ^{m}C_{ds}A_{ds}T_{kS}TₖₛAₖ | |
| 1061305 | 4 | 4 | 2′-OMe | CXCL12 | Gₖₛ^{m}CₖₛAₖₛT_{ds}G_{ds}T_{ds}Tₘₓ^{m}C_{ds}T_{ds}^{m}C_{ds}A_{ds} | 18 |
| | | | | | ^{m}C_{ds}A_{ds}T_{kS}TₖₛAₖ | |
| 1061306 | 1 | 1 | 2′-OMe | SOD1 | TₖₛGₖₛAₖₛGₘₓG_{ds}T_{ds}^{m}C_{ds}^{m}C_{ds}T_{ds}G_{ds}^{m}C_{ds} | 26 |
| | | | | | A_{ds}^{m}C_{ds}TₖₛGₖₛGₖ | |
| 1061307 | 2 | 2 | 2′-OMe | SOD1 | TₖₛGₖₛAₖₛG_{ds}GₘₓT_{ds}^{m}C_{ds}^{m}C_{ds}T_{ds}G_{ds}^{m}C_{ds} | 26 |
| | | | | | A_{ds}^{m}C_{ds}TₖₛGₖₛGₖ | |
| 1061308 | 3 | 3 | 2′-OMe | SOD1 | TₖₛGₖₛAₖₛG_{ds}G_{ds}Tₘₓ^{m}C_{ds}^{m}C_{ds}T_{ds}G_{ds}^{m}C_{ds} | 26 |
| | | | | | A_{ds}^{m}C_{ds}TₖₛGₖₛGₖ | |
| 955900 | 3 | 1 | 2′-FANA | CXCL12 | Gₖₛ^{m}Ck_{S}Ak_{S}U_{(FANA)s}G_{ds}T_{dx} | 19 |
| | | | | | T_{ds}^{m}C_{ds}T_{ds}^{m}C_{ds}A_{ds}^{m}C_{ds}A_{ds}TₖₛTₖₛAₖ | |
| 955901 | 3 | 2 | 2′-FANA | CXCL12 | GₖₛmCₖₛAₖₛT_{ds}G_{(FANA)s}T_{dx} | 18 |
| | | | | | T_{ds}^{m}C_{ds}T_{ds}^{m}C_{ds}A_{ds}^{m}C_{ds}A_{ds}TₖₛTₖₛAₖ | |
| 955902 | 3 | 4 | 2′-FANA | CXCL12 | GₖₛmCₖₛAₖₛT_{ds}G_{ds}T_{dx} | 21 |
| | | | | | U_{(FANA)s}^{m}C_{ds}T_{ds}^{m}C_{ds}A_{ds}^{m}C_{ds}A_{ds}TₖₛTₖₛAₖ | |
| 955903 | 3 | 5 | 2′-FANA | CXCL12 | Gₖₛ^{m}CₖₛAₖₛT_{ds}G_{ds}T_{dx}T_{ds}C(_{FANA)s}T_{ds}^{m}C_{ds} | 18 |
| | | | | | A_{ds}^{m}C_{ds}A_{ds}TₖₛTₖₛAₖ | |
| 955904 | 3 | 6 | 2′-FANA | CXCL12 | Gₖₛ^{m}CₖₛAₖₛT_{ds}G_{ds}T_{dx}T_{ds}C_{ds}U_{(FANA)s)} | 39 |
| | | | | | ^{m}C_{ds}A_{ds}^{m}C_{ds}A_{ds}TₖₛTₖₛAₖ | |
| 955905 | 3 | 7 | 2′-FANA | CXCL12 | Gₖₛ^{m}CₖₛAₖₛT_{ds}G_{ds}T_{dx}T_{ds}^{m}C_{ds}T_{ds}C_{(FANA)s} | 18 |
| | | | | | A_{ds}C_{ds}AₐₛTₖₛTₖₛAₖ | |
| 955906 | 3 | 8 | 2′-FANA | CXCL12 | Gₖₛ^{m}C_{kS}AₖₛT_{ds}G_{ds}T_{dx}T_{ds}^{m}C_{ds}T_{ds}^{m}C_{ds} | 18 |
| | | | | | A_{(FANA)s}C_{ds}A_{ds}TₖₛTₖₛAₖ | |
| 955907 | 3 | 9 | 2′-FANA | CXCL12 | Gₖₛ^{m}CₖₛAₖₛT_{ds}G_{ds}T_{dx}T_{ds}^{m}C_{ds}T_{ds}^{m}C_{ds}A_{ds} | 18 |
| | | | | | C_{(FANA)s}A_{ds}TₖₛTₖₛAₖ | |
| 955908 | 3 | 10 | 2′-FANA | CXCL12 | Gₖₛ^{m}CₖₛAₖₛT_{ds}G_{ds}T_{dx}T_{ds}^{m}C_{ds}T_{ds}^{m}C_{ds}A_{ds} | 18 |
| | | | | | ^{m}C_{ds}A_{(FANA)s}TₖₛTₖₛAₖ | |
| 855156 | n/a | n/a | n/a | CXCL12 | Gₖₛ^{m}CₖₛAₖₛT_{ds}G_{ds}T_{ds}T_{ds}^{m}C_{ds}T_{ds}^{m}C_{ds}A_{ds} | 18 |
| | | | | | ^{m}C_{ds}A_{ds}TₖₛTₖₛAₖ-HPPO-GalNac | |
| 855161 | 3 | n/a | n/a | CXCL12 | GₖₛmCₖₛAₖₛT_{ds}G_{ds}T_{dx} | 18 |
| | | | | | T_{ds}^{m}C_{ds}T_{ds}^{m}C_{ds}A_{ds}^{m}C_{ds}A_{ds}TₖₛTₖₛAₖ- | |
| | | | | | HPPO-GalNac | |
| 855160 | 1, 2 | n/a | n/a | CXCL12 | Gₖₛ^{m}CₖₛAₖₛT_{dx}G_{dx}T_{ds} | 18 |
| | | | | | T_{ds}^{m}C_{ds}T_{ds}^{m}C_{ds}A_{ds}^{m}C_{ds}A_{ds}TₖₛTₖₛAₖ- | |
| | | | | | HPPO-GalNac | |
| 895571 | n/a | 3 | 2′-MOE | CXCL12 | Gₖₛ^{m}CₖₛAₖₛT_{ds}G_{ds}TₑₛT_{ds}^{m}C_{ds}T_{ds}^{m}C_{ds}A_{ds} | 18 |
| | | | | | ^{m}C_{ds}A_{ds}TₖₛTₖₛAₖ-HPPO-GalNac | |
| 978782 | 3 | 2 | 2′-FANA | CXCL12 | Gₖₛ^{m}CₖₛAₖₛT_{ds}G_{(FANA)s}T_{dx} | 18 |
| | | | | | T_{ds}^{m}C_{ds}T_{ds}^{m}C_{ds}A_{ds}^{m}C_{ds}A_{ds}TₖₛTₖₛAₖ- | |
| | | | | | HPPO-GalNac | |
| 978783 | 3 | 4 | 2′-FANA | CXCL12 | Gₖₛ^{m}CₖₛAₖₛT_{ds}G_{ds}T_{dx} | 21 |
| | | | | | U_{(FANA)s}^{m}C_{ds}T_{ds}^{m}C_{ds}A_{ds}^{m}C_{ds}A_{ds}TₖₛTₖₛAₖ- | |
| | | | | | HPPO-GalNac | |
| 978784 | 3 | 5 | 2′-FANA | CXCL12 | GₖₛmCₖₛAₖₛT_{ds}G_{ds}T_{dx} | 18 |
| | | | | | T_{ds}C_{(FANA)s}T_{ds}mC_{ds}A_{ds}^{m}C_{ds}A_{ds}TₖₛTₖₛAₖ- | |
| | | | | | HPPO-GalNac | |
| 978785 | 3 | 6 | 2′-FANA | CXCL12 | Gₖₛ^{m}CₖₛAₖₛT_{ds}G_{ds}T_{dx} | 39 |
| | | | | | T_{ds}^{m}C_{ds}U_{(FANA)s}^{m}C_{ds}A_{ds}^{m}C_{ds}A_{ds}TₖₛTₖₛAₖ- | |
| | | | | | HPPO-GalNac | |
| 978786 | 3 | 10 | 2′-FANA | CXCL12 | Gₖₛ^{m}CₖₛAₖₛT_{ds}G_{ds}T_{dx} | 18 |
| | | | | | T_{ds}^{m}C_{ds}T_{ds}^{m}C_{ds}A_{ds}^{m}C_{ds}A_{(FANA)s}TₖₛTₖₛAₖ- | |
| | | | | | HPPO-GalNac | |

A subscript “d” indicates an unmodified, 2′-β-D-(eoxyribosyl sugar moiety.
A subscript “e” indicates a 2′-MOE-β-D-ribofuranosyl sugar moiety.
A subscript “m” indicates a 2′-O-methyl-β-D-ribofuranosyl sugar moiety.
A subscript “k” indicates a cEt.
A subscript “s” indicates a phosphorothioate internucleoside linkage.
A subscript “f” indicates a 2′-F-β-D-2′-deoxyribosyl sugar moiety.
A subscript “(FANA)” indicates a 2′-F-β-D-arabinofuranosyl modified sugar moiety.
A superscript “m” before a C indicates 5-methyl Cytosine.

For in vitro activity studies, 3T3-L1 cells were plated at 20,000 cells/well and transfected with 0, 27 nM, 82 nM, 247 nM, 740 nM, 2,220 nM, 6,660 nM or 20,000 nM of modified oligonucleotide by electroporation. After X hours, mRNA was harvested and analyzed by RT-qPCR. Target and raptor mRNA was analyzed as previous examples.

TABLE 26
Effect of MOP backbone modifications combined with ara-F
modifications on in vitro activity and toxicity

| | MOP | FANA | | |
|---|---|---|---|---|
| | position | position | Target | RAPTOR |
| Compound | in central | in central | IC₅₀ | IC₅₀ |
| ID | region | region | (nM) | (nM) |
| 558807 | n/a | n/a | 34 | 1000 |
| 766678 | 3 | n/a | 67 | >20,000 |
| 955900 | 3 | 1 | 58 | >20,000 |
| 955901 | 3 | 2 | 43 | >20,000 |
| 955902 | 3 | 4 | 27 | >20,000 |
| 955903 | 3 | 5 | 27 | >20,000 |
| 955904 | 3 | 6 | 65 | >20,000 |
| 955905 | 3 | 7 | 93 | 16000 |
| 955906 | 3 | 8 | 99 | >20,000 |
| 955907 | 3 | 9 | 154 | >20,000 |
| 955908 | 3 | 10 | 171 | >20,000 |

For the in vivo toxicity study in the table below, three male BALB/C mice per modified oligonucleotide were administered 0.2, 0.6, 1.8 or 50 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity.

TABLE 27
Effect of MOP backbone modifications combined with
2′ modifications on in vivo activity and toxicity

| | | position | | | |
|---|---|---|---|---|---|
| | | of 2′- | sugar | | |
| | MOP | altered | modification | in vivo | |
| Com- | position | nucleotide | of 2′- | CXCL12 | |
| pound | in central | in central | altered | ED₅₀ | ALT @ |
| ID | region | region | nucleotide | (mg/kg) | 50 mg/kg |
| 855156 | n/a | n/a | n/a | 0.13 | 2938* |
| 855161 | 3 | n/a | n/a | 0.36 | 40 |
| 855160 | 2, 3 | n/a | n/a | 0.37 | 28 |
| 895571 | n/a | 3 | 2′-MOE | 0.43 | 319 |
| 978782 | 3 | 2 | 2′-FANA | 0.47 | 56 |
| 978783 | 3 | 4 | 2′-FANA | 0.43 | 39 |

*Value represents the ALT at 1.8 mg/kg

For the in vivo toxicity study in the table below, male BALB/C mice per modified oligonucleotide were administered 5, 50, or 150 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity. The caspase assay was performed in vitro as described in Example 8.

TABLE 28
Effect of MOP backbone modifications combined
with 2′-OMe modified sugar moieties

| | MOP | 2′-OMe | | | | |
|---|---|---|---|---|---|---|
| | position | position | | in vivo | ALT | ALT |
| Com- | in | in | CXCL12 | CXCL12 | @ 50 | @ 150 |
| pound | central | central | IC₅₀ | ED₅₀ | mg/kg | mg/kg |
| ID | region | region | (μM) | (mg/kg) | (IU/L) | (IU/L) |
| 558807 | n/a | n/a | 0.18 | 2.88 | 8329 | death |
| 936053 | n/a | 2 | 0.17 | 1.75 | 75 | 40 |
| 1061302 | 1 | 1 | 0.09 | 0.39 | 101 | 2253 |
| 1061303 | 2 | 2 | 0.13 | 11 | 49 | 34 |
| 1061304 | 3 | 3 | 0.09 | 4.6 | 31 | 52 |
| 1061305 | 4 | 4 | 0.09 | 15.4 | 22 | 31 |

TABLE 28b
in vitro Caspase Activation

| | | | | | 2,222 | 6,667 | 20,000 |
|---|---|---|---|---|---|---|---|
| Compound | 27 nM | 80 nM | 250 nM | 740 nM | nM | nM | nM |
| ID | Relative Caspase Activation (% Control) | | | | | | |
| 558807 | 98 | 106 | 112 | 139 | 288 | 587 | 1977 |
| 936053 | 106 | 111 | 113 | 91 | 98 | 107 | 153 |
| 1061302 | 98 | 90 | 106 | 111 | 149 | 456 | 1555 |
| 1061303 | 104 | 99 | 104 | 84 | 102 | 86 | 125 |
| 1061304 | 91 | 97 | 82 | 96 | 85 | 105 | 269 |
| 1061305 | 90 | 96 | 72 | 91 | 84 | 103 | 348 |

TABLE 28c
In Vivo Dose-response of Gadd45a mRNA upon treatment with
modified oligonucleotides complementary to CXCL12

| Compound | 5.0 mg/kg | 50 mg/kg | 150 mg/kg |
|---|---|---|---|
| ID | Expression level of Gadd45a mRNA (% Control) | | |
| 558807 | 120 | 473 | n.d. |
| 936053 | 76 | 169 | 219 |
| 1061302 | 188 | 178 | 357 |
| 1061303 | 55 | 66 | 66 |
| 1061304 | 58 | 66 | 97 |
| 1061305 | 67 | 18 | 20 |

TABLE 28d
In Vivo Dose-response of Tnfrsf10b mRNA upon treatment
with modified oligonucleotides complementary to CXCL12

| Compound | 5.0 mg/kg | 50 mg/kg | 150 mg/kg |
|---|---|---|---|
| ID | Expression level of Tnfrsf10b mRNA (% Control) | | |
| 558807 | 137 | 8022 | n.d. |
| 936053 | 91 | 104 | 180 |
| 1061302 | 104 | 137 | 1217 |
| 1061303 | 90 | 92 | 110 |
| 1061304 | 70 | 75 | 149 |
| 1061305 | 79 | 60 | 50 |

TABLE 28e
In Vivo Dose-response of P21 mRNA upon treatment with
modified oligonucleotides complementary to CXCL12

| Compound | 5.0 mg/kg | 50 mg/kg | 150 mg/kg |
|---|---|---|---|
| ID | Expression level of P21 mRNA (% Control) | | |
| 558807 | 84 | 58138 | n.d. |
| 936053 | 239 | 81 | 299 |
| 1061302 | 87 | 315 | 14680 |
| 1061303 | 293 | 495 | 480 |
| 1061304 | 182 | 400 | 353 |
| 1061305 | 353 | 321 | 223 |

### Example 13 Effect of Position-Specific 2′-OMe on In Vitro Activity and Toxicity of Modified Oligonucleotides with a Variety of Sequences

Modified oligonucleotides were synthesized as indicated in the table below. The compounds have the sugar motif kkk-d(10)-kkk or kkk-d-m-d(8)-kkk, where “k” represents a cEt, “d” represents an unmodified, 2′-β-D-deoxyribosyl sugar moiety, and “m” represents a 2′-OMe-β-D-ribofuranosyl sugar moiety. For sequences with a T at position 5 (from the 5′ end) in the parent kkk-d(10)-kkk oligonucleotide, the kkk-d-m-d(8)-kkk contains a 2′-OMe modified U at this position. For sequences with a ^{m}C at position 5 (from the 5′ end) in the parent kkk-d(10)-kkk oligonucleotide, the kkk-d-m-d(8)-kkk contains a 2′-OMe modified C at this position, with no 5-Me group.

For the in vivo toxicity study in the table below, two male BALB/C mice per modified oligonucleotide were administered 150 mg/kg modified oligonucleotide by subcutaneous injection and sacrificed 72 hours later. One animal was administered an injection of saline as a control. Plasma levels of ALT was measured using an automated clinical chemistry analyzer. Increased ALT is indicative of acute liver toxicity.

For a subset of compounds, on-target activity was measured in the liver. RNA was isolated from the liver and measured by RT-qPCR using the primer probe sets described in Table 30 below. Results were normalized with Ribogreen® and are reported normalized to PBS-treated animals.

Levels of mRNA for Gadd45a, P21, and Tnfrsf10b were analyzed as in Example 1 for mice administered 150 mg/kg modified oligonucleotide. Results are normalized with Ribogreen® and presented relative to PBS-treated control animals.

The caspase assay was performed in vitro as described in Example 8.

TABLE 29
Targets and Sequences

| | Corresponding | | | |
|---|---|---|---|---|
| | compound with 2′- | | | |
| Parent | OMe at position 2 | | | |
| Compound | of the | Complementary | | SEQ |
| ID | central region | mRNA | Sequence | ID NO |
| 546006 | 1133071 | HDAC2 | GAGGATGGCAAGCACA | 41 |
| 549334 | | AR | CACCTGCGGGAAGCTC | 42 |
| | 1200896 | AR | CACCUGCGGGAAGCTC | 126 |
| 562920 | 1201379 | VWF | TGTGCCCCAGCCCATT | 43 |
| 572912 | 1200898 | PABPN1 | CTTCCACAGTATATCT | 44 |
| 576095 | 1200899 | EGLN2 | TACTGGTAGTGTTGCT | 45 |
| 597605 | 1200900 | HEGFL | TTGACACAAAGGGAGT | 46 |
| 601840 | 1201381 | MTDH | GAATCTCCTTTTCCAG | 47 |
| 640599 | 1201862 | EZH2 | TTTACACGCTTCCGCC | 48 |
| 694804 | | DNM2 | AGACTCTCGGTTCCGA | 49 |
| | 1202810 | | AGACUCTCGGTTCCGA | 127 |
| 738431 | 1200905 | Nestin | CTTTTCTATCAGTCTC | 51 |
| 739428 | | WWTR1/TNS | CTTCTTGATGTCTTTC | 52 |
| | 1201694 | | CTTCUTGATGTCTTTC | 129 |
| 747137 | 1200907 | FOXO1A | AAGTGTCACTAAAACC | 53 |
| 747149 | | FOXO1A | GGACTGAAATAGCAGA | 54 |
| | 1203759 | | GGACUGAAATAGCAGA | 130 |
| 747190 | | FOXO1A | AGGCTGGCCCCCACTG | 55 |
| | 1203759 | | AGGCUGGCCCCCACTG | 131 |
| 758252 | | CHOP/DDIT3 | GGTTTTTGATTCTTCC | 56 |
| | 1203759 | | GGTTUTTGATTCTTCC | 132 |
| 797793 | 1201073 | DLL4 | GCATGCCGCCCCGTCC | 57 |
| 808013 | 1203761 | CYBB | TCTTCATACAATAGCA | 58 |
| 813942 | 1203762 | CDK9 | CGTTCAAATTCCGTCT | 59 |
| 832311 | 1201199 | PEMT | TCCGGCTGCGGCTCAG | 60 |

TABLE 30
Primer Probe Sets

| | | | | SEQ |
|---|---|---|---|---|
| | PP Set | | | ID |
| Transcript | Name | Primer | Sequence | NO: |
| HDAC2 | RTS3500 | Forward | TGATGGTGTTGAGGAAGCTTTTT | 15 |
| | | Reverse | TCCCTCAAGTCTCCTGTTCCA | 16 |
| | | Probe | ACAACAGATCGCGTGATGACCGTCTC | 17 |
| DNM2 | RTS36436 | Forward | AGAGGAGACCGAGCGAAT | 61 |
| | | Reverse | CATGGTTTGTGTTGATGTACGAC | 62 |
| | | Probe | CCTACATCAGGGAGCGAGAAGGGA | 63 |
| FOXO1A | RTS4973 | Forward | GTCAAGACTACAACACACAGC | 64 |
| | | Reverse | AAAACTATAAGGAGGGGTGAAGG | 65 |
| | | Probe | CTGAAGGACTTTTAAATGTAGCCTG | 66 |
| | | | CTCACTAA | |
| PABPN1 | n/a | Forward | CCGGAGCTAGAAGCGATCAA | 70 |
| | | Reverse | CCTTTAGCTTCTCAGCCTCTTCCT | 71 |
| | | Probe | CTCGAGTCAGGGAGATG | 72 |

TABLE 31
Toxicity and Activity

| | Position of | | | | | | Relative | |
|---|---|---|---|---|---|---|---|---|
| | 2′-altered | sugar | | | | | Caspase | |
| | nucleotide | modification | Gadd45a | Tnfrsf10b | | | Activation | Complementary |
| Compound | in central | of altered | mRNA | mRNA | P21 mRNA | ALT @ | (% Control) | mRNA* |
| ID | region | nucleotide | (% Control) | (% Control) | (% Control) | 150 mg/kg | @ 20 μM | (% Control) |
| PBS | n/a | n/a | 100 | 100 | 111 | 28 @ 0 | n.d. | 100 |
| 546006 | n/a | n/a | 1885 | 4660 | 6556 | 131 | 291 | 38 |
| 1133071 | 2 | 2′-OMe | 1357 | 5569 | 6541 | 245 | 279 | 37 |
| 549334 | n/a | n/a | 187 | 225 | 182 | 30 | n.d. | n.d. |
| 1200896 | 2 | 2′-OMe | 165 | 126 | 130 | 28 | n.d. | n.d. |
| 562920 | n/a | n/a | 498 | 568 | 1336 | 109 | 473 | n.d. |
| 1201379 | 2 | 2′-OMe | 171 | 200 | 402 | 32 | 473 | n.d. |
| 572912 | n/a | n/a | 357 | 7503 | 5043 | 3883 | 205 | n.d. |
| 1200898 | 2 | 2′-OMe | 155 | 170 | 301 | 41 | 84 | n.d. |
| 576095 | n/a | n/a | 147 | 121 | 154 | 39 | n.d. | n.d. |
| 1200899 | 2 | 2′-OMe | 401 | 154 | 169 | 32 | n.d. | n.d. |
| 597605 | n/a | n/a | 353 | 1965 | 2263 | 488 | 328 | n.d. |
| 1200900 | 2 | 2′-OMe | 121 | 227 | 228 | 32 | 126 | n.d. |
| 601840 | n/a | n/a | 221 | 365 | 840 | 98 | 287 | n.d. |
| 1201381 | 2 | 2′-OMe | 103 | 123 | 72 | 24 | 274 | n.d. |
| 640599 | n/a | n/a | 111 | 286 | 376 | 26 | 184 | n.d. |
| 1201862 | 2 | 2′-OMe | 96 | 262 | 276 | 22 | 99 | n.d. |
| 694804 | n/a | n/a | 336 | 916 | 1297 | 1090 | 257 | 6 |
| 1202810 | 2 | 2′-OMe | 106 | 238 | 257 | 36 | 166 | 16 |
| 715415 | n/a | n/a | 186 | 1211 | 1249 | 420 | 137 | n.d. |
| 1203758 | 2 | 2′-OMe | 78 | 150 | 115 | 41 | 141 | n.d. |
| 738431 | n/a | n/a | 229 | 507 | 448 | 608 | 220 | n.d. |
| 1200905 | 2 | 2′-OMe | 141 | 193 | 197 | 69 | 181 | n.d. |
| 739428 | n/a | n/a | 234 | 1975 | 2107 | 533 | 269 | n.d. |
| 1201694 | 2 | 2′-OMe | 154 | 593 | 388 | 42 | 114 | n.d. |
| 747137 | n/a | n/a | 155 | 1379 | 1851 | 50 | 512 | 19 |
| 1200907 | 2 | 2′-OMe | 99 | 716 | 824 | 39 | 168 | 27 |
| 747149 | n/a | n/a | 454 | 5765 | 4892 | 606 | 166 | 9 |
| 1203759 | 2 | 2′-OMe | 105 | 119 | 211 | 33 | 109 | 22 |
| 747190 | n/a | n/a | 162 | 2856 | 4677 | 1315 | 393 | 2 |
| 1200961 | 2 | 2′-OMe | 129 | 237 | 345 | 71 | 305 | 7 |
| 758252 | n/a | n/a | 158 | 989 | 861 | 725 | 355 | n.d. |
| 1233760 | 2 | 2′-OMe | 94 | 106 | 182 | 47 | 187 | n.d. |
| 797793 | n/a | n/a | 190 | 1175 | 1181 | 1318 | 229 | n.d. |
| 1201073 | 2 | 2′-OMe | 184 | 230 | 201 | 78 | 125 | n.d. |
| 808013 | n/a | n/a | 126 | 2153 | 4617 | 169 | 437 | n.d. |
| 1203761 | 2 | 2′-OMe | 154 | 163 | 147 | 25 | 113 | n.d. |
| 813942 | n/a | n/a | 351 | 3758 | 4638 | 127 | 340 | n.d. |
| 1203762 | 2 | 2′-OMe | 103 | 89 | 257 | 28 | 88 | n.d. |
| 832311 | n/a | n/a | 305 | 1059 | 878 | 739 | 288 | n.d. |
| 1201199 | 2 | 2′-OMe | 294 | 720 | 597 | 208 | 256 | n.d. |

*Value represents the reduction of the mRNA that is complementary to the modified oligonucleotide as indicated in Table 29 above.

### Example 14 Dose-Response of Position-Specific 2′-OMe on In Vitro and In Vivo Activity and Toxicity of Modified Oligonucleotides with a Variety of Sequences

On target in vivo activity and toxicity was measured for a subset of compounds described in Example 13 above. Two male BALB/c mice per group were administered 1.85, 5.55, 16.67, 50, or 150 mg/kg modified oligonucleotide once via subcutaneous injection, as indicated in the tables below. Mice were sacrificed after 1 week and mRNA was isolated from the liver and measured by RT-qPCR using the primer probe sets described in Table 30 above. Levels of mRNA for Gadd45a, P21, and Tnfrsf10b were analyzed as in Example 1. Results were normalized with Ribogreen and are reported normalized to PBS-treated animals. Plasma levels of ALT was measured using an automated clinical chemistry analyzer. Increased ALT is indicative of acute liver toxicity. The therapeutic index is calculated as a ratio of the maximum non-toxic dose divided by the in vivo ED50. The maximum non-toxic dose is the highest dose at which the ALT value remains less than 5× increased compared to PBS-treated mice, typically 150 IU/L.

TABLE 33
in vivo dose response activity

| | Target Expression (% Control) | | | | | |
|---|---|---|---|---|---|---|
| Compound | 1.9 | 5.6 | 16.7 | 50 | 150 | ED50 |
| Number | mg/kg | mg/kg | mg/kg | mg/kg | mg/kg | (μg/g) |
| 546006 | 86.8 | 75.6 | 78.1 | 56.2 | 41.4 | 87.9 |
| 1133071 | 81.6 | 74.5 | 61.4 | 52.9 | 37.6 | 53.9 |
| 572912 | 80.7 | 58.2 | 34.6 | 26.2 | 21.7 | 10.0 |
| 1200898 | 84.8 | 80.2 | 67.4 | 42.4 | 16.3 | 31.5 |
| 694804 | 62.8 | 38.2 | 15.3 | 7.8 | 7.6 | 3.2 |
| 1202810 | 71.0 | 46.7 | 33.2 | 21.0 | 10.1 | 5.8 |
| 747137 | 45.3 | 42.5 | 28.7 | 21.4 | 14.0 | 1.5 |
| 1200907 | 42.1 | 35.3 | 40.2 | 30.6 | 18.8 | 0.53 |
| 747149 | 72.8 | 42.2 | 23.6 | 14.6 | 7.3 | 6.0 |
| 1203759 | 52.9 | 40.6 | 24.4 | 21.5 | 18.3 | 2.1 |
| 715415 | 61.5 | 56.9 | 41.9 | 19.9 | 12.0 | 6.5 |
| 1203758 | 71.6 | 68.8 | 61.1 | 34.8 | 20.9 | 20.0 |

TABLE 34
in vivo dose response toxicity (ALT)

| | ALT (IU/L) | | | | |
|---|---|---|---|---|---|
| Compound | 1.9 | 5.6 | 16.7 | 50 | 150 |
| Number | mg/kg | mg/kg | mg/kg | mg/kg | mg/kg |
| 546006 | 29 | 29 | 22 | 26 | 173 |
| 1133071 | 24 | 25 | 25 | 44 | 356 |
| 572912 | 23 | 27 | 112 | 730 | 4674 |
| 1200898 | 23 | 24 | 25 | 28 | 32 |
| 694804 | 29 | 24 | 24 | 143 | 2160 |
| 1202810 | 22 | 24 | 24 | 23 | 61 |
| 747137 | 24 | 22 | 24 | 25 | 86 |
| 1200907 | 23 | 21 | 21 | 31 | 32 |
| 747149 | 26 | 26 | 38 | 157 | 1867 |
| 1203759 | 25 | 21 | 23 | 27 | 27 |
| 715415 | 23 | 21 | 25 | 77 | 1384 |
| 1203758 | 25 | 23 | 23 | 23 | 54 |

TABLE 34b
In Vivo Dose-response of Gadd45a mRNA upon treatment
with modified oligonucleotides complementary to HDAC2

| | 1.9 | 5.6 | 16.7 | 50 | 150 |
|---|---|---|---|---|---|
| | mg/kg | mg/kg | mg/kg | mg/kg | mg/kg |
| | Expression level of Gadd45a mRNA (% Control) | | | | |
| 546006 | 122 | 112 | 77 | 302 | 1070 |
| 1133071 | 94 | 134 | 154 | 396 | 873 |
| 572912 | 94 | 95 | 187 | 196 | 227 |
| 1200898 | 103 | 80 | 112 | 109 | 99 |
| 694804 | 126 | 106 | 161 | 163 | 459 |
| 1202810 | 115 | 93 | 91 | 188 | 169 |
| 747137 | 94 | 67 | 80 | 96 | 153 |
| 1200907 | 79 | 86 | 142 | 88 | 140 |
| 747149 | 123 | 172 | 146 | 283 | 575 |
| 1203759 | 100 | 147 | 102 | 172 | 154 |
| 715415 | 91 | 118 | 201 | 159 | 393 |
| 1203758 | 143 | 114 | 206 | 162 | 197 |

TABLE 34c
In Vivo Dose-response of Tnfrsf10b mRNA upon
treatment with modified oligonucleotides

| | 1.9 | 5.6 | 16.7 | 50 | 150 |
|---|---|---|---|---|---|
| Compound | mg/kg | mg/kg | mg/kg | mg/kg | mg/kg |
| ID | Expression level of Tnfrsf10b mRNA (% Control) | | | | |
| 546006 | 87 | 78 | 161 | 248 | 4235 |
| 1133071 | 95 | 96 | 157 | 622 | 5166 |
| 572912 | 210 | 190 | 551 | 4070 | 5847 |
| 1200898 | 135 | 116 | 105 | 170 | 179 |
| 694804 | 81 | 98 | 116 | 284 | 1775 |
| 1202810 | 88 | 110 | 88 | 128 | 241 |
| 747137 | 56 | 74 | 115 | 273 | 1013 |
| 1200907 | 99 | 86 | 15 | 239 | 453 |
| 747149 | 73 | 70 | 116 | 636 | 6027 |
| 1203759 | 87 | 55 | 57 | 97 | 105 |
| 715415 | 62 | 57 | 111 | 259 | 999 |
| 1203758 | 67 | 72 | 64 | 79 | 126 |

TABLE 34d
In Vivo Dose-response of P21 mRNA upon treatment
with modified oligonucleotides

| | 1.9 | 5.6 | 16.7 | 50 | 150 |
|---|---|---|---|---|---|
| Compound | mg/kg | mg/kg | mg/kg | mg/kg | mg/kg |
| ID | Expression level of P21 mRNA (% Control) | | | | |
| 546006 | 103 | 90 | 172 | 342 | 5193 |
| 1133071 | 97 | 68 | 197 | 773 | 6571 |
| 572912 | 224 | 228 | 762 | 2787 | 3182 |
| 1200898 | 98 | 106 | 70 | 164 | 172 |
| 694804 | 108 | 76 | 72 | 172 | 2212 |
| 1202810 | 91 | 125 | 245 | 51 | 162 |
| 747137 | 43 | 59 | 122 | 294 | 1220 |
| 1200907 | 108 | 97 | 110 | 383 | 708 |
| 747149 | 95 | 44 | 207 | 985 | 3869 |
| 1203759 | 61 | 30 | 47 | 71 | 95 |
| 715415 | 46 | 24 | 45 | 213 | 757 |
| 1203758 | 36 | 34 | 18 | 26 | 35 |

TABLE 34e
Therapeutic Index

| | 2′-altered | sugar | | | |
|---|---|---|---|---|---|
| | nucleotide | modifica- | | | |
| | position | tion of | | TI | |
| Compound | in central | altered | MNTD | (MNTD/ | Fold-TI |
| ID | region | nucleotide | (mg/kg) | ED₅₀) | improvement |
| 546006 | n/a | n/a | 50 | 0.6 | 1.5 |
| 1133071 | 2 | 2′-OMe | 50 | 0.9 | |
| 572912 | n/a | n/a | 16.7 | 2 | >2.4 |
| 1200898 | 2 | 2′-OMe | >150 | >4.8 | |
| 694804 | n/a | n/a | 50 | 16 | >1.6 |
| 1202810 | 2 | 2′-OMe | >150 | >26 | |
| 747137 | n/a | n/a | >150 | >99 | ~2.9 |
| 1200907 | 2 | 2′-OMe | >150 | >284 | |
| 747149 | n/a | n/a | 50 | 11 | >6.5 |
| 1203759 | 2 | 2′-OMe | >150 | >72 | |
| 715415 | n/a | n/a | 50 | 8 | n/a |
| 1203758 | 2 | 2′-OMe | >150 | >7.5 | |

### Example 15 Effect of Modified Oligonucleotides on Nucleolar Localization of p54Nrb

Selected modified nucleotides described in above were tested for their effect on HeLa cells by microscopy. HeLa cells were transfected by lipofectamine 2000 with 200 nM of modified oligonucleotide for 2 hrs and then cellular protein p54nrb was stained by mP54 antibody (Santa Cruz Biotech, sc-376865) and DAPI was used to stain for the nucleus of cells. The number of cells with nucleolar p54nrb and the total number of cells in the images were counted. ALT data presented were previously described in Example 13 above.

TABLE 35
Nucleolar mislocalization of p54nrb
and correlation with hepatoxicity

| | 2′-altered | | | |
|---|---|---|---|---|
| | nucleotide | sugar | | |
| | position | modification | % cells with | |
| Compound | in central | of altered | mislocalization | ALT @ |
| ID | region | nucleotide | of p54nrb | 150 mg/kg |
| 546006 | n/a | n/a | 56 | 131 |
| 1133071 | 2 | 2′-OMe | 67 | 245 |
| 572912 | n/a | n/a | 75 | 3883 |
| 1200898 | 2 | 2′-OMe | 3 | 41 |
| 758252 | n/a | n/a | 71 | 725 |
| 1233760 | 2 | 2′-OMe | 4 | 47 |

### Example 16 Effect of Position-Specific 2′-OMe on In Vitro Activity and Toxicity of Modified Oligonucleotides with Various Sequences

Modified oligonucleotides with the sugar motifs lll-d(10)-lll and lll-d-m-d(8)-lll were synthesized, where “l” indicates a β-D-locked nucleic acid (β-D-LNA), “d” represents a 2′-β-D-deoxyribosyl sugar moiety and “m” represents a 2′-OMe-β-D-ribofuranosyl sugar moiety. Each internucleoside linkage is a phosphorothioate internucleoside linkage. For sequences with a T at position 5 (from the 5′ end) in the parent lll-d(10)-lll oligonucleotide, the lll-d-m-d(8)-lll contains a 2′-OMe modified U at this position. For sequences with a ^{m}C at position 5 (from the 5′ end) in the parent lll-d(10)-lll oligonucleotide, the lll-d-m-d(8)-lll contains a 2′-OMe modified C at this position lacking a 5-Me group.

TABLE 36
Modified Oligonucleotides

| lll-d(10)- | lll-d-m- | | | |
|---|---|---|---|---|
| 111 | d(8)-lll | Comple- | | SEQ |
| compound | compound | mentary | | ID |
| ID | ID | mRNA | Sequence (5′-3′) | NO |
| 1247569 | 1247570 | HDAC2 | GAGGATGGCAAGCACA | 41 |
| 1247571 | 1247572 | PABPN1 | CTTCCACAGTATATCT | 44 |
| 1247573 | | DNM2 | AGACTCTCGGTTCCGA | 49 |
| | 1247574 | | AGACUCTCGGTTCCGA | 127 |
| 1247575 | 1247576 | FOXO1A | AAGTGTCACTAAAACC | 53 |
| 1247577 | 1247578 | FOXO1A | GGACTGAAATAGCAGA | 54 |
| | 1247578 | | GGACUGAAATAGCAGA | 130 |

In vivo toxicity and on target in vivo activity was measured for the compounds described above. Two male balb/c mice per group were administered 16.67 or 150 mg/kg modified oligonucleotide once via subcutaneous injection, as indicated in the tables below. Mice were sacrificed after 72 hours and mRNA was isolated from the liver and measured by RT-qPCR using the primer probe sets described in Table 30 above. Levels of mRNA for P21, and Tnfrsf10b were analyzed as in Example 1. Results were normalized with Ribogreen and are reported normalized to PBS-treated animals.

TABLE 37
in vivo activity and toxicity

| | Complementary | | | | | | | |
|---|---|---|---|---|---|---|---|---|
| | mRNA | | | | | | Tnfrsf10b | |
| | Expression | | ALT | | P21 mRNA | | mRNA | |
| Com- | (% Control) | | (IU/L) | | (% Control) | | (% Control) | |
| pound | 16.7 | 150 | 16.7 | 150 | 16.7 | 150 | 16.7 | 150 |
| Number | mg/kg | mg/kg | mg/kg | mg/kg | mg/kg | mg/kg | mg/kg | mg/kg |
| 1247569 | 70 | 35 | 37 | 2368 | 163 | 12778 | 158 | 7046 |
| 1247570 | 72 | 46 | 34 | 867 | 444 | 11860 | 320 | 6772 |
| 1247571 | 40 | 26 | 460 | 10838 | 3061 | 7588 | 2216 | 8133 |
| 1247572 | 54 | 16 | 26 | 330 | 90 | 928 | 124 | 679 |
| 1247573 | 7 | 19 | 59 | 20665 | 153 | 10379 | 157 | 4858 |
| 1247574 | 19 | 6 | 25 | 284 | 139 | 839 | 123 | 575 |
| 1247575 | 51 | 30 | 50 | 2404 | 390 | 11275 | 334 | 6365 |
| 1247576 | 57 | 27 | 25 | 85 | 142 | 1850 | 218 | 2033 |
| 1247577 | 52 | 25 | 34 | 2460 | 256 | 11736 | 193 | 14610 |
| 1247578 | 60 | 21 | 25 | 39 | 124 | 133 | 178 | 143 |
| 1247579 | 48 | 14 | 23 | 1696 | 95 | 3704 | 176 | 108 |
| 1247580 | 77 | 21 | 28 | 232 | 78 | 265 | 2850 | 307 |

### Example 17 Effect of Position-Specific 2′-Modifications on In Vitro Activity and In Vivo Liver Toxicity of Modified Oligonucleotides Complementary to CPT1A

Modified oligonucleotides were synthesized as indicated in the table below. These modified oligonucleotides were compared to the otherwise identical modified oligonucleotide lacking an altered nucleotide in the central region, a 5-10-5 MOE modified oligonucleotide, containing five nucleosides each comprising a 2′-MOE-β-D-ribofuranosyl sugar moiety in each of the 5′ and 3′ regions and 10 unmodified DNA nucleosides in the central region. Each internucleoside linkage is a phosphorothioate internucleoside linkage. The compounds in the table below are 100% complementary to mouse CPT1A, GENBANK NC_000085.6 truncated from 3319001 to 3389000 (SEQ ID NO: 6), at position 49870 to 49889. In certain instances, a modified oligonucleotide comprising a T at position 5 is compared to a modified oligonucleotide comprising a 2′-OMe U at position 5.

TABLE 38
Modified oligonucleotides complementary to CPT1A

| | position of | | | |
|---|---|---|---|---|
| | altered | sugar | | |
| | nucleotide | modification | | SEQ |
| Compound | in central | of altered | | ID |
| ID | region | nucleotide | Chemistry Notation | NO |
| 147420 | n/a | n/a | AₑₛAₑₛTₑₛGₑₛTₑₛG_{ds}^{m}C_{ds}^{m}C_{ds}T_{ds}G_{ds}^{m}C_{ds}T_{ds} | 73 |
| | | | G_{ds}T_{ds}^{m}C_{ds}^{m}CₑₛTₑₛTₑₛGₑₛAₑ | |
| 994947 | n/a | OMe | AₑₛAₑₛTₑₛGₘₛTₑₛG_{ds}^{m}C_{ds}^{m}C_{ds}T_{ds}G_{ds}^{m}C_{ds}T_{ds} | 73 |
| | | | G_{ds}T_{ds}^{m}C_{ds}^{m}CₑₛTₑₛTₑₛGₑₛAₑ | |
| 994948 | n/a | OMe | AₑₛAₑₛTₑₛGₑₛUₘₛG_{ds}^{m}C_{ds}^{m}C_{ds}T_{ds}G_{ds}^{m}C_{ds}T_{ds} | 74 |
| | | | G_{ds}T_{ds}^{m}C_{ds}^{m}CₑₛTₑₛTₑₛGₑₛAₑ | |
| 994949 | 1 | OMe | AₑₛAₑₛTₑₛGₑₛTₑₛGₘₛ^{m}C_{ds}^{m}C_{ds}T_{ds}G_{ds}^{m}C_{ds}T_{ds} | 73 |
| | | | G_{ds}T_{ds}^{m}C_{ds}^{m}CₑₛTₑₛTₑₛGₑₛAₑ | |
| 994950 | 2 | OMe | AₑₛAₑₛTₑₛGₑₛTₑₛG_{ds}Cₘₛ^{m}C_{ds}T_{ds}G_{ds}^{m}C_{ds}T_{ds} | 73 |
| | | | G_{ds}T_{ds}^{m}C_{ds}^{m}CₑₛTₑₛTₑₛGₑₛAₑ | |
| 994951 | 3 | OMe | AₑₛAₑₛTₑₛGₑₛTₑₛG_{ds}^{m}C_{ds}CₘₛT_{ds}G_{ds}^{m}C_{ds}T_{ds} | 73 |
| | | | G_{ds}T_{ds}^{m}C_{ds}^{m}CₑₛTₑₛTₑₛGₑₛAₑ | |

A subscript “d” indicates an unmodified, 2′-β-D-deoxyribosyl sugar moiety.
A subscript “e” indicates a 2′-MOE-β-D-ribofuranosyl sugar moiety.
A subscript “m” indicates a 2′-O-methyl-β-D-ribofuranosyl sugar moiety.
A subscript “k” indicates a cEt. A subscript “s” indicates a phosphorothioate internucleoside linkage.
A superscript “m” before a C indicates 5-methyl Cytosine..
A subscript “x” indicates a MOP internucleoside linkage.
A subscript “(FANA)” indicates a nucleoside comprising an ara 2′-F modified sugar moiety.

For the in vivo toxicity and activity study in the table below, BALB/C mice per modified oligonucleotide were administered 200 mg/kg modified oligonucleotide by subcutaneous injection and sacrificed 72 hours later. One animal was administered an injection of saline as a control. Plasma levels of ALT were measured using an automated clinical chemistry analyzer. Increased ALT is indicative of acute liver toxicity. Liver mRNA was harvested and analyzed by RT-qPCR. Cpt1a mRNA was detected using primer probe setRTS40014 (forward sequence: AGATCAATCGGACCCTAGACA, SEQ ID NO: 75; reverse sequence: CAGCACCTTCAGCGAGTA; SEQ ID NO: 76; probe sequence: AAGAGGACGCCACTCACGATGTTC, SEQ ID NO: 77) and P21 and Tnfrsf10b mRNA were detected as described in Example 1.

TABLE 39
Activity and toxicity of modified oligonucleotides complementary CPT1A

| | position | sugar | | | | |
|---|---|---|---|---|---|---|
| | of altered | modification | | | | ALT @ |
| Compound | nucleotide in | of altered | Cpt1a | P21 | Tnfrsf10b | 200 mg/kg |
| ID | central region | nucleotide | (% control) | (% control) | (% control) | (IU/L) |
| 147420 | n/a | n/a | 6.42 | 6616 | 8796 | 15308 |
| 994947 | n/a | 2′-OMe | 6.49 | 6984 | 11499 | 18395 |
| 994948 | n/a | 2′-OMe | 8.99 | 7085 | 10520 | 10535 |
| 994949 | 1 | 2′-OMe | 5.90 | 6370 | 9595 | 12370 |
| 994950 | 2 | 2′-OMe | 12.19 | 2219 | 2146 | 52 |
| 994951 | 3 | 2′-OMe | 6.72 | 6275 | 10555 | 2991 |

For the in vivo activity study in the table below, three BALB/C mice per modified oligonucleotide were administered 2.5, 7.4, 22.2, 66.7, 200 mg/kg 147420 or 994950 by subcutaneous injection and sacrificed after 72 hours. Plasma levels of ALT were measured using an automated clinical chemistry analyzer. Increased ALT is indicative of acute liver toxicity. Cpt1a mRNA was detected using RT-qPCR as described above.

TABLE 40
In Vivo Toxicity of modified oligonucleotides
complementary CPT1A

| | ALT (IU/L) | | | | |
|---|---|---|---|---|---|
| Compound | 2.5 | 7.4 | 22.2 | 66.7 | 200 |
| Number | mg/kg | mg/kg | mg/kg | mg/kg | mg/kg |
| 147420 | 51 | 23 | 23 | 106 | 7794 |
| 994950 | 25 | 25 | 21 | 23 | 53 |

TABLE 41
In Vivo Activity of modified oligonucleotides
complementary CPT1A

| | CPT1 mRNA (% Control) | | | | |
|---|---|---|---|---|---|
| Compound | 2.5 | 7.4 | 22.2 | 66.7 | 200 |
| Number | mg/kg | mg/kg | mg/kg | mg/kg | mg/kg |
| 147420 | 115.9 | 93.1 | 78.2 | 33.7 | 4.6 |
| 994950 | 116.0 | 117.2 | 105.6 | 55.9 | 21.9 |

TABLE 42
In Vivo Dose-response of Tnfrsf10b mRNA upon
treatment with modified oligonucleotides

| | Tnfrsf10b mRNA (% Control) | | | | |
|---|---|---|---|---|---|
| Compound | 2.5 | 7.4 | 22.2 | 66.7 | 200 |
| Number | mg/kg | mg/kg | mg/kg | mg/kg | mg/kg |
| 147420 | 132 | 141 | 162 | 2341 | 8622 |
| 994950 | 119 | 133 | 125 | 153 | 1026 |

TABLE 43
In Vivo Dose-response of P21 mRNA upon treatment
with modified oligonucleotides

| | P21 mRNA (% Control) | | | | |
|---|---|---|---|---|---|
| Compound | 2.5 | 7.4 | 22.2 | 66.7 | 200 |
| Number | mg/kg | mg/kg | mg/kg | mg/kg | mg/kg |
| 147420 | 141 | 94 | 240 | 4305 | 15334 |
| 994950 | 105 | 89 | 103 | 208 | 2413 |

### Example 18 Effect of 2′-OMe Modification in Modified Oligonucleotides Complementary to Factor XI

Modified oligonucleotides were synthesized as indicated in the table below. The compounds have the sugar motif kkk-d(10)-kkk (“parent”) or kkk-d-m-d(8)-kkk, where “k” represents a cEt, “d” represents an unmodified, 2′-β-D-deoxyribosyl sugar moiety, and “m” represents a 2′-OMe-ribofuranosyl sugar moiety. For sequences with a T at position 5 (from the 5′ end) in the parent kkk-d(10)-kkk oligonucleotide, the kkk-d-m-d(8)-kkk contains a 2′-OMe modified U at this position. For sequences with a ^{m}C at position 5 (from the 5′ end) in the kkk-d(10)-kkk oligonucleotide, the kkk-d-m-d(8)-kkk contains a 2′-OMe modified C at this position, lacking the 5-Me group. Each internucleoside linkage is a phosphorothioate internucleoside linkage.

For the in vitro activity study in the table below, primary mouse hepatocytes were isolated from male balb/c mice and transfected with 0.9, 2.7, 8.2, 24.7, 74.0, 222, 667, or 2,000 nM modified oligonucleotide. After 24 hrs, mRNA was harvested and analyzed for FXI and RAPTOR as described above.

For the in vivo toxicity study in the table below, two male BALB/C mice per modified oligonucleotide were administered 150 mg/kg modified oligonucleotide by subcutaneous injection and sacrificed 72 hours later. One animal was administered an injection of saline as a control. Plasma levels of ALT was measured using an automated clinical chemistry analyzer. Increased ALT is indicative of acute liver toxicity.

For a subset of compounds, on-target activity was measured in the liver. RNA was isolated from the liver and measured by RT-qPCR using the primer probe set RTS2898, described in Example 4 above. Results were normalized with Ribogreen® and are reported normalized to PBS-treated animals.

TABLE 44
Sequences

| kkk-d(10)- | | | |
|---|---|---|---|
| kkk | kkk-d-m-d(8)- | | |
| compound | kkk compound | | SEQ |
| ID | ID | Sequence (5′-3′) | ID NO |
| 464917 | 982034 | GTCTGTGCATCTCTCC | 22 |
| 464924 | | GTTATTGTGGTTGGCG | 81 |
| | 1133247 | GTTAUTGTGGTTGGCG | 133 |
| 465156 | | ATTCTGTGTGCACTGC | 82 |
| | 1133310 | ATTCUGTGTGCACTGC | 134 |
| 465162 | 1133316 | TCTTGTCTGACATTCT | 83 |
| 465163 | 1133317 | TTTTGTGTCTTCTGTA | 84 |
| 465172 | | CTGTTTGAGTTTTCTC | 85 |
| | 1133326 | CTGTUTGAGTTTTCTC | 135 |
| 465174 | 1133328 | CAAAGTGATACCAGTT | 86 |
| 465175 | | AATCTTCCAGGGCCAC | 87 |
| | 1133329 | AATCUTCCAGGGCCAC | 136 |
| 465176 | | TCATTTCTATGGAATA | 88 |
| | 1133330 | TCATUTCTATGGAATA | 137 |
| 465178 | 1133332 | GTCAGTATCCCAGTGT | 89 |
| 465179 | 1133333 | GGTTACAGTGGAAGAG | 90 |
| 465181 | 1133335 | TCTGGGTGTTCTTACG | 91 |
| 465186 | 1133340 | TTTCCTTGAGTAGTAG | 92 |
| 465187 | 1133341 | TCTCCTTGCTGTATTT | 93 |

TABLE 45
Activity and Toxicity of Modified oligonucleotides complementary to Factor XI

| | | | | | Relative | | | |
|---|---|---|---|---|---|---|---|---|
| | altered | sugar | | in vitro | Caspase | | | |
| | nucleotide | modification | in vitro | RAPTOR | Activation | p21 | FXI | ALT @ |
| Compound | position in | of altered | FXI IC₅₀ | IC₅₀ | (% Control) | @150 | @ 10 | 150 mg/kg |
| ID | central region | nucleotide | (nM) | (nM) | @20 μM | mg/kg | mg/kg | (IU/L) |
| 464917 | n/a | n/a | 101 | 44 | 700 | death | 25.4 | death |
| 982034 | 2 | 2′-OMe | 221 | 119 | 122 | 31922 | 40.8 | 13172 |
| 464924 | n/a | n/a | 115 | >2000 | 332 | 19340 | 8.6 | 5365 |
| 1133247 | 2 | 2′-OMe | 189 | >2000 | 190 | 753 | 12.1 | 33 |
| 465156 | n/a | n/a | 98 | 129 | 934 | 82279 | 14.4 | 24858 |
| 1133310 | 2 | 2′-OMe | 354 | 465 | 603 | 127 | 14.7 | 7034 |
| 465162 | n/a | n/a | 99 | >2000 | 758 | death | 15.1 | death |
| 1133316 | 2 | 2′-OMe | 144 | >2000 | 189 | 4660 | 34.2 | 60 |
| 465163 | n/a | n/a | 163 | >2000 | 115 | 34117 | 41.5 | 2347 |
| 1133317 | 2 | 2′-OMe | 272 | >2000 | 67 | 11844 | 79.4 | 478 |
| 465172 | n/a | n/a | 106 | >2000 | 429 | 512 | 57.6 | 23 |
| 1133326 | 2 | 2′-OMe | 176 | >2000 | 157 | 237 | 64.8 | 20 |
| 465174 | n/a | n/a | 69 | >2000 | 130 | 276 | 21.7 | 21 |
| 1133328 | 2 | 2′-OMe | 393 | >2000 | 113 | 167 | 33.8 | 23 |
| 465175 | n/a | n/a | 50 | 125 | 523 | 6957 | 37.8 | 1564 |
| 1133329 | 2 | 2′-OMe | 99 | 170 | 501 | 1564 | 59.8 | 60 |
| 465176 | n/a | n/a | 111 | >2000 | 219 | 344 | 98.6 | 27 |
| 1133330 | 2 | 2′-OMe | 89 | >2000 | 135 | 190 | 95.1 | 22 |
| 465178 | n/a | n/a | 11 | 115 | 364 | 900086 | 8.1 | 13168 |
| 1133332 | 2 | 2′-OMe | 24 | 1653 | 247 | 5982 | 27.0 | 75 |
| 465179 | n/a | n/a | 74 | >2000 | 188 | 4046 | 23.0 | 344 |
| 1133333 | 2 | 2′-OMe | 82 | >2000 | 102 | 122 | 62.4 | 23 |
| 465181 | n/a | n/a | 75 | 1571 | 487 | 17469 | 25.4 | 7087 |
| 1133335 | 2 | 2′-OMe | 56 | >2000 | 214 | 929 | 61.7 | 26 |
| 465186 | n/a | n/a | 75 | >2000 | 200 | 42551 | 17.3 | 3709 |
| 1133340 | 2 | 2′-OMe | 208 | >2000 | 125 | 513 | 42.8 | 34 |
| 465187 | n/a | n/a | 35 | 475 | 393 | 778834* | 10.4 | 11752* |
| 1133341 | 2 | 2′-OMe | 28 | >2000 | 167 | 1984 | 38.6 | 36 |

*½ animals were found dead

### Example 19 Effect of 2′-OMe Incorporation on Toxicity of Modified Oligonucleotides Complementary to HDAC2

Modified oligonucleotides were synthesized as indicated in the table below. The compounds have the sugar motif kkk-d(10)-kkk (“parent”) or kkk-d-m-d(8)-kkk, where “k” represents a cEt, “d” represents an unmodified, 2′-β-D-deoxyribosyl sugar moiety, and “m” represents a 2′-OMe-β-D-ribofuranosylsugar moiety. For sequences with a T at position 5 (from the 5′ end) in the parent kkk-d(10)-kkk oligonucleotide, the kkk-d-m-d(8)-kkk contains a 2′-OMe modified U at this position. For sequences with a mC at position 5 (from the 5′ end) in the kkk-d(10)-kkk oligonucleotide, the kkk-d-m-d(8)-kkk contains a 2′-OMe modified C at this position, lacking the 5-Me group. Each internucleoside linkage is a phosphorothioate internucleoside linkage.

For the in vitro activity study in the table below, primary mouse hepatocytes were isolated from male balb/c mice and transfected with 0.9, 2.7, 8.2, 24.7, 74.0, 222, 667, or 2,000 nM modified oligonucleotide. After 24 hrs, mRNA was harvested and analyzed for HDAC2 and RAPTOR as described above. For the in vivo toxicity study in the table below, two male BALB/C mice per modified oligonucleotide were administered 10 or 150 mg/kg modified oligonucleotide by subcutaneous injection and sacrificed 72 hours later. One animal was administered an injection of saline as a control. Plasma levels of ALT was measured using an automated clinical chemistry analyzer. Increased ALT is indicative of acute liver toxicity.

For a subset of compounds, on-target activity was measured in the liver. RNA was isolated from the liver and measured by RT-qPCR using the primer probe set RTS3500 described above in Example 13. Results were normalized with Ribogreen® and are reported normalized to PBS-treated animals.

TABLE 46
Sequences

| kkk-d(10)- | | | |
|---|---|---|---|
| kkk | kkk-d-m-d(8)- | | |
| compound | kkk compound | | SEQ |
| ID | ID | Sequence (5′-3′) | ID NO |
| 545984 | 1133060 | TTGCCAATATCACCAT | 94 |
| 545996 | | CAACUGAACCACCCGT | 95 |
| | 1133066 | CAACTGAACCACCCGT | 138 |
| 546004 | 1133070 | GCACAATATCATTAAC | 96 |
| 546009 | 1132933 | GACTCTCTGATGATAC | 97 |
| 546023 | 1132940 | CTATACCATCTCTCAT | 98 |
| 546024 | 1133080 | CATCATCTATACCATC | 99 |
| 546034 | 1133085 | ACACATTTAGCATGAC | 100 |
| 546045 | | ATTATATGGCAACTCA | 101 |
| | 1132951 | ATTAUATGGCAACTCA | 139 |
| 546049 | 1132953 | GACTAATATGCAGTTT | 102 |
| 546075 | 1132966 | GTCAAATTCAAGGGTT | 103 |
| 546095 | 1132976 | CATAAAGCATGGTGGA | 104 |
| 546108 | 1133122 | TAGTCTCTGTCAGTTA | 105 |
| 546109 | 1132983 | GTACCTATAGTCTCTG | 106 |
| 546110 | 1133123 | TCATGTACCTATAGTC | 107 |
| 546112 | 1133124 | TCTTAATTTCATGTAC | 108 |
| 546118 | 1133127 | ACCCTCAAGTCTCCTG | 109 |

TABLE 47
In vitro Activity and Toxicity of Modified
oligonucleotides complementary to HDAC2

| | altered | sugar | | | Relative |
|---|---|---|---|---|---|
| | nucleotide | modifica- | | | Caspase |
| Com- | position in | tion of | in vitro | in vitro | Activation |
| pound | central | altered | HDAC 2 | RAPTOR | (% Control) |
| ID | region | nucleotide | IC₅₀ (nM) | IC₅₀ (nM) | @20 μM |
| 546009 | n/a | n/a | 44 | 773 | 632 |
| 1132933 | 2 | 2′-OMe | 89 | >2000 | 193 |
| 546023 | n/a | n/a | 79 | 1643 | 825 |
| 1132940 | 2 | 2′-OMe | 156 | >2000 | 980 |
| 546045 | n/a | n/a | 72 | 460 | 469 |
| 1132951 | 2 | 2′-OMe | 98 | >2000 | 326 |
| 546049 | n/a | n/a | 82 | >2000 | 127 |
| 1132953 | 2 | 2′-OMe | 144 | >2000 | 132 |
| 546075 | n/a | n/a | 81 | >2000 | 149 |
| 1132966 | 2 | 2′-OMe | 135 | >2000 | 143 |
| 546095 | n/a | n/a | 78 | >2000 | 126 |
| 1132976 | 2 | 2′-OMe | 128 | >2000 | 98 |
| 546109 | n/a | n/a | 30 | >2000 | 396 |
| 1132983 | 2 | 2′-OMe | 44 | >2000 | 117 |
| 545984 | n/a | n/a | 89 | 452 | 1235 |
| 1133060 | 2 | 2′-OMe | 126 | >2000 | 270 |
| 545996 | n/a | n/a | 297 | >2000 | 776 |
| 1133066 | 2 | 2′-OMe | 111 | >2000 | 327 |
| 546004 | n/a | n/a | 181 | >2000 | 124 |
| 1133070 | 2 | 2′-OMe | 164 | >2000 | 90 |
| 546024 | n/a | n/a | 85 | >2000 | 124 |
| 1133080 | 2 | 2′-OMe | 45 | >2000 | 123 |
| 546034 | n/a | n/a | 125 | >2000 | 107 |
| 1133085 | 2 | 2′-OMe | 125 | >2000 | 104 |
| 546108 | n/a | n/a | 21 | 144 | 1265 |
| 1133122 | 2 | 2′-OMe | 34 | >2000 | 176 |
| 546110 | n/a | n/a | 17 | >2000 | 82 |
| 1133123 | 2 | 2′-OMe | 30 | >2000 | 95 |
| 546112 | n/a | n/a | 178 | >2000 | 106 |
| 1133124 | 2 | 2′-OMe | 106 | >2000 | 98 |
| 546118 | n/a | n/a | 6 | 181 | 425 |
| 1133127 | 2 | 2′-OMe | 11 | >2000 | 158 |

TABLE 48
In vivo Activity and Toxicity of Modified
oligonucleotides complementary to HDAC2

| | | | P21 | | ALT |
|---|---|---|---|---|---|
| | HDAC2 | HDAC2 | mRNA | Tnfrsf10b | @ 150 |
| Compound | @ 10 | @ 150 | @ 150 | @ 150 | mg/kg |
| ID | mg/kg | mg/kg | mg/kg | mg/kg | (IU/L) |
| 546009 | 22 | 7.5 | 5739 | 7162 | 14907 |
| 1132933 | 40 | 7.5 | 98 | 176 | 31 |
| 546023 | 57 | 9.9 | 1586 | 754 | 820 |
| 1132940 | 61 | 12.7 | 1348 | 565 | 224 |
| 546045 | 68 | 17.2 | 5601 | 2362 | 1031 |
| 1132951 | 60 | 15.3 | 1726 | 970 | 183 |
| 546049 | 50 | 8.9 | 294 | 133 | 29 |
| 1132953 | 71 | 15.2 | 282 | 150 | 27 |
| 546075 | 71 | 16.2 | 282 | 232 | 21 |
| 1132966 | 61 | 27.7 | 741 | 621 | 86 |
| 546095 | 39 | 12.7 | 3303 | 2314 | 1063 |
| 1132976 | 50 | 15.3 | 685 | 512 | 94 |
| 546109 | 29 | 4.3 | 684 | 706 | 182 |
| 1132983 | 37 | 5.2 | 217 | 190 | 34 |
| 545984 | 31 | 4.9 | 14070 | 10327 | 37277 |
| 1133060 | 42 | 9.0 | 183 | 138 | 39 |
| 545996 | 56 | 14.7 | 613 | 458 | 433 |
| 1133066 | 60 | 24.2 | 215 | 156 | 28 |
| 546004 | 64 | 13.4 | 499 | 203 | 35 |
| 1133070 | 69 | 17.6 | 286 | 192 | 30 |
| 546024 | 34 | 6.2 | 381 | 169 | 25 |
| 1133080 | 41 | 8.1 | 452 | 201 | 26 |
| 546034 | 52 | 7.5 | 181 | 140 | 32 |
| 1133085 | 68 | 10.6 | 127 | 143 | 27 |
| 546108 | 3 | n.d. | n.d. | n.d. | death |
| 1133122 | 7 | 1.9 | 1524 | 1353 | 131 |
| 546110 | 15 | 6.2 | 23642 | 6298 | 5132 |
| 1133123 | 35 | 3.3 | 221 | 155 | 29 |
| 546112 | 52 | 14.3 | 817 | 350 | 34 |
| 1133124 | 59 | 13.9 | 822 | 571 | 29 |
| 546118 | 13 | 5.7 | 3853 | 3854 | 3894 |
| 1133127 | 15 | 4.8 | 470 | 473 | 139 |

For the FOB scores reported in the table below, mice per group were administered 100 μg modified oligonucleotide by intracerebroventricular (ICV) injection. At 3 hours and 2 weeks post-injection, mice were evaluated according to 7 different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not. After all 7 criteria were evaluated, the FOB scores were summed for each mouse and averaged within each treatment group. The results are presented in the table below.

Two weeks after injection of modified oligonucleotide, mice were sacrificed and levels of HDAC, p21 and Aif1 were measured in the cortex and the spinal cord by RT-PCR as described above. Aif1 is a marker for inflammation. Results are presented below relative to control animals.

TABLE 48b
In vivo Activity and Toxicity of Modified
oligonucleotides complementary to HDAC2

| | | | | HDAC | | p21 | | Aif1 |
|---|---|---|---|---|---|---|---|---|
| | | | HDAC | mRNA | p21 | mRNA | Aif1 | mRNA |
| Compound | 3 hr | 2 week | mRNA | Spinal | mRNA | Spinal | mRNA | Spinal |
| ID | FOB | FOB | Cortex | Cord | Cortex | Cord | Cortex | cord |
| 546009 | 5.5 | 5.5 | n.d. | n.d. | n.d. | n.d. | n.d. | n.d. |
| 1132933 | 3.5 | 0 | 45.3 | 45.3 | 148 | 227 | 96 | 129 |
| 546023 | 0 | 0 | 36.3 | 31.3 | 120 | 144 | 117 | 156 |
| 1132940 | 0 | 0 | 59.2 | 39.3 | 135 | 166 | 94 | 174 |
| 546045 | 4.5 | 0 | 43.4 | 39.2 | 136 | 284 | 113 | 161 |
| 1132951 | 3 | 0 | 61.4 | 42.6 | 128 | 200 | 86 | 121 |
| 546049 | 1 | 0 | 95.3 | 68.8 | 111 | 116 | 90 | 110 |
| 1132953 | 3 | 0 | 67.1 | 47.8 | 126 | 138 | 82 | 103 |
| 546075 | 7 | 7 | n.d. | n.d. | n.d. | n.d. | n.d. | n.d. |
| 1132966 | 2.5 | 0 | 39.9 | 40.9 | 129 | 174 | 116 | 130 |
| 546095 | 5.5 | 0 | 66.7 | 44.2 | 124 | 321 | 90 | 189 |
| 1132976 | 6 | 0 | 55 | 36 | 132 | 427 | 87 | 248 |
| 546109 | 7 | 7 | n.d. | n.d. | n.d. | n.d. | n.d. | n.d. |
| 1132983 | 7 | 7 | n.d. | n.d. | n.d. | n.d. | n.d. | n.d. |
| 545984 | 2.5 | 6.5 | n.d. | n.d. | n.d. | n.d. | n.d. | n.d. |
| 1133060 | 1 | 0 | 71.0 | 39.8 | 107 | 130 | 92 | 112 |
| 545996 | 3 | 0 | 59.1 | 48.9 | 122 | 220 | 104 | 171 |
| 1133066 | 7 | 7 | n.d. | n.d. | n.d. | n.d. | n.d. | n.d. |
| 546004 | 0 | 0 | 55.3 | 47.6 | 136 | 261 | 116 | 176 |
| 1133070 | 0 | 0 | 58.7 | 50.0 | 127 | 173 | 81 | 99 |
| 546024 | 6.5 | 0 | 20.2 | 20.5 | 134 | 211 | 115 | 140 |
| 1133080 | 1 | 0 | 34.6 | 18.4 | 109 | 139 | 88 | 112 |
| 546034 | 3 | 0 | 58.9 | 46.6 | 96 | 149 | 98 | 146 |
| 1133085 | 3 | 0 | 79.0 | 42.0 | 114 | 126 | 101 | 137 |
| 546108 | 2 | 6.5 | n.d. | n.d. | n.d. | n.d. | n.d. | n.d. |
| 1133122 | 3.5 | 1 | 25.4 | 17.7 | 97 | 166 | 120 | 178 |
| 546110 | 2.5 | 3 | 55.0 | 23.5 | 88 | 294 | 93 | 342 |
| 1133123 | 0 | 0 | 57.4 | 49.8 | 112 | 149 | 85 | 105 |
| 546112 | 3 | 0 | 68.5 | 46.6 | 108 | 119 | 97 | 420 |
| 1133124 | 1.5 | 0 | 70.8 | 52.8 | 122 | 107 | 157 | 873 |
| 546118 | 7 | 7 | n.d. | n.d. | n.d. | n.d. | n.d. | n.d. |
| 1133127 | 7 | 7 | n.d. | n.d. | n.d. | n.d. | n.d. | n.d. |

### Example 20 Incorporation of 2′-OMe at Various Positions

Modified oligonucleotides were synthesized with kkk-m-d(9)-kkk, kkk-d-m-d(8)-kkk, kkk-dd-m-d(7)-kkk or kkk-d(3)-m-d(6)-kkk sugar motifs, respectively, where “m” represents a 2′-OMe-β-D-ribofuranosylsugar moiety, “k” represents a cEt, and “d” represents an unmodified, 2′-β-D-deoxyribosyl sugar moiety. In certain instances, 2′-OMeU replaces 2′-deoxyT. These modified oligonucleotides were compared to the otherwise identical modified oligonucleotide lacking an altered nucleotide in the central region, a 3-10-3 cEt modified oligonucleotide, containing three cEt nucleosides in each of the 5′ and 3′ regions and 10 unmodified DNA nucleosides in the central region. Each internucleoside linkage is a phosphorothioate internucleoside linkage. The compounds in the table below are 10000 complementary to mouse CXCL12, GENBANK NT_039353.7 truncated from 69430515 to 69445350 (SEQ ID NO: 1), at position 6877 to 6892.

TABLE 49
Modified Oligonucleotides

| | altered | | | |
|---|---|---|---|---|
| | nucleotide | sugar moiety | | SEQ |
| Compound | position in | of altered | | ID |
| ID | central region | nucleotide | Chemistry Notation | NO |
| 558807 | n/a | n/a | Gₖₛ^{m}CₖₛAₖₛT_{ds}G_{ds}T_{ds}T_{ds}^{m}C_{ds}T_{ds}^{m} | 18 |
| | | | C_{ds}A_{ds}^{m}C_{ds}A_{ds}TₖₛTₖₛAₖ | |
| 1061877 | 1 | 2′-OMe | Gₖₛ^{m}CₖₛAₖₛUₘₛG_{ds}T_{ds}T_{ds}^{m}C_{ds}T_{ds}^{m} | 19 |
| | | | C_{ds}A_{ds}^{m}C_{ds}A_{ds}TₖₛTₖₛAₖ | |
| 936053 | 2 | 2′-OMe | Gₖₛ^{m}CₖₛAₖₛT_{ds}GₘₛT_{ds}T_{ds}^{m}C_{ds}T_{ds}^{m} | 18 |
| | | | C_{ds}A_{ds}^{m}C_{ds}A_{ds}TₖₛTₖₛAₖ | |
| 1061879 | 3 | 2′-OMe | Gₖₛ^{m}CₖₛAₖₛT_{ds}G_{ds}UₘₛT_{ds}^{m}C_{ds}T_{ds}^{m} | 20 |
| | | | C_{ds}A_{ds}^{m}C_{ds}A_{ds}TₖₛTₖₛAₖ | |
| 1061981 | 4 | 2′-OMe | Gₖₛ^{m}C_{kS}A_{kS}T_{ds}G_{ds}T_{ds}Uₘₛ^{m}C_{ds}T_{ds}^{m} | 21 |
| | | | C_{ds}A_{ds}^{m}C_{ds}A_{ds}TₖₛTₖₛAₖ | |
| 1244110 | 5 | 2′-OMe | Gₖₛ^{m}CₖₛAₖₛT_{ds}G_{ds}T_{ds}T_{ds}CₘₛT_{ds}^{m} | 18 |
| | | | C_{ds}A_{ds}^{m}C_{ds}A_{ds}T_{kS}TₖₛAₖ | |
| 1244111 | 6 | 2′-OMe | GₖₛmCₖₛAₖₛT_{ds}G_{ds}T_{ds}T_{ds}mC_{ds}Uₘₛ^{m} | 39 |
| | | | C_{ds}A_{ds}^{m}C_{ds}A_{ds}TₖₛT_{kS}Aₖ | |
| 1244112 | 7 | 2′-OMe | Gₖₛ^{m}CₖₛAₖₛT_{ds}G_{ds}T_{ds}T_{ds}^{m}C_{ds}T_{ds} | 18 |
| | | | CₘₛA_{ds}^{m}C_{ds}A_{ds}T_{kS}TₖₛAₖ | |
| 1244113 | 8 | 2′-OMe | Gₖₛ^{m}CₖₛAₖₛT_{ds}G_{ds}T_{ds}T_{ds}^{m}C_{ds}T_{ds}^{m} | 18 |
| | | | C_{ds}Aₘₛ^{m}C_{ds}A_{ds}TₖₛTₖₛAₖ | |
| 1244114 | 9 | 2′-OMe | Gₖₛ^{m}CₖₛAₖₛT_{ds}G_{ds}T_{ds}T_{ds}^{m}C_{ds}T_{ds}^{m} | 18 |
| | | | C_{ds}A_{ds}CₘₛA_{ds}TₖₛTₖₛAₖ | |
| 1244115 | 10 | 2′-OMe | Gₖₛ^{m}CₖₛAₖₛT_{ds}G_{ds}T_{ds}T_{ds}^{m}C_{ds}T_{ds}^{m} | 18 |
| | | | C_{ds}A_{ds}^{m}C_{ds}AₘₛTₖₛTₖₛAₖ | |

A subscript “d” indicates an unmodified, 2′-β-D-deoxyribosyl sugar moiety. A subscript “in” indicates a 2′-methyl-β-D-ribofuranosyl sugar moiety. A subscript “k” indicates a cEt. A subscript “s” indicates a phosphorothioate internucleoside linkage.. A superscript “in” before a C indicates 5-methyl Cytosine.

For the results in the tables below, in vitro activity and toxicity experiments were performed essentially as described in Example 1. For in vitro activity and toxicity studies, 3T3-L1 cells were transfected with 27 nM, 80 nM, 250 nM, 740 nM, 2, 222 nM, 6,667 nM, or 20,000 nM of modified oligonucleotide by electroporation and levels of p21 were measured by RT-qPCR as described in Example 1 above. The caspase assay was performed as described in Example 8 above in 3T3-L1 cells. Localization of p54nrb in HeLa cells was quantitated as described in Example 15.

TABLE 50
In vitro activity and toxicity of modified
oligonucleotides complementary to CXCL12

| | in vitro | in vitro | in vitro | |
|---|---|---|---|---|
| | CXCL12 | Caspase | p21 | % |
| Compound | IC₅₀ | (% control) | (% control) | nucleolar |
| ID | (nM) | @ 20 μM | @ 20 μM | p54nrb |
| 558807 | 47 | 641 | 307 | 92 |
| 1061877 | 13 | 519 | 266 | 43 |
| 936053 | 67 | 173 | 143 | 8 |
| 1061879 | 59 | 416 | 192 | 59 |
| 1061981 | 112 | 325 | 129 | 46 |
| 1244110 | 21 | 386 | 390 | 60 |
| 1244111 | 53 | 380 | 430 | 69 |
| 1244112 | 42 | 345 | 344 | 92 |
| 1244113 | 114 | 361 | 373 | 54 |
| 1244114 | 17 | 399 | 440 | 78 |
| 1244115 | 70 | 372 | 400 | 67 |

### Example 21 Stereochemical Isomers of Nucleosides

Modified oligonucleotides containing modified nucleotides with various stereochemical configurations at positions 1′, 3′, and 5′ of the 2′-deoxyfuranosyl sugar were synthesized. Amidites for the synthesis of β-L-DNA-containing nucleotides are commercially available (ChemGenes) and the synthesis of both α-L and β-L dT phosphoramidites has been reported (Morvan, Biochem and Biophys Research Comm, 172(2): 537-543, 1990). The altered nucleotides were contained within the central region of the oligonucleotide.

These modified oligonucleotides were compared to the otherwise identical modified oligonucleotide lacking a an altered nucleotide in the central region, 558807, described in Table 1, Example 1 above. The compounds in Table 51 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. The compounds in the table below are 100% complementary to mouse CXCL12, GENBANK NT_039353.7 truncated from 69430515 to 69445350 (SEQ ID NO: 1), at position 6877 to 6892.

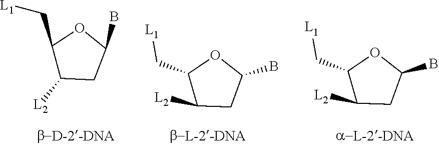

B is any nucleobase and L₁ and L₂ are internucleoside linkages

TABLE 51
modified oligonucleotides with stereochemical modifications

| | position of | | | |
|---|---|---|---|---|
| | altered | stereochemical | | |
| | nucleotide | configuration | | |
| Compound | in central | of altered | | SEQ ID |
| ID | region | nucleotide | Chemistry Notation (5′ to 3′) | NO |
| 1215458 | 2 | β-L-DNA | Gₖₛ^{m}CₖₛAₖₛT_{ds}[_{β-L}G_{ds}]T_{ds} | 18 |
| | | | T_{ds}^{m}C_{ds}T_{ds}^{m}C_{ds}A_{ds}^{m}C_{ds}A_{ds}TₖₛTₖₛAₖ | |
| 1215459 | 3 | β-L-DNA | Gₖₛ^{m}CₖₛAₖₛT_{ds}G_{ds} | 18 |
| | | | [_{β-L}T_{ds}]T_{ds}^{m}C_{ds}T_{ds}^{m}C_{ds}A_{ds}^{m}C_{ds}A_{ds}TₖₛTₖₛAₖ | |
| 1215460 | 4 | β-L-DNA | Gₖₛ^{m}CₖₛAₖₛT_{ds}G_{ds}T_{ds} | 18 |
| | | | [_{β-L}T_{ds}]^{m}C_{ds}T_{ds}^{m}C_{ds}A_{ds}^{m}C_{ds}A_{ds}TₖₛTₖₛAₖ | |
| 1215461 | 3 | α-L-DNA | Gₖₛ^{m}CₖₛAₖₛT_{ds}G_{ds} | 18 |
| | | | [_{α-L}T_{ds}]T_{ds}^{m}C_{ds}T_{ds}^{m}C_{ds}A_{ds}^{m}C_{ds}A_{ds}TₖₛTₖₛAₖ | |
| 1215462 | 4 | α-L-DNA | Gₖₛ^{m}CₖₛAₖₛT_{ds}G_{ds}T_{ds} | 18 |
| | | | [_{α-L}T_{ds}]^{m}C_{ds}T_{ds}^{m}C_{ds}A_{ds}^{m}C_{ds}A_{ds}TₖₛTₖₛAₖ | |

A subscript “d” indicates an unmodified, 2′-β-D-deoxyribosyl sugar moiety. A subscript “k” indicates a cEt. A subscript “s” indicates a phosphorothioate internucleoside linkage. [_{β-L}B_{ds}] indicates a modified β-L-DNA nucleotide with a 2′-deoxyribosyl moiety, a phosphorothioate linkage, and base B. [_{α-L}B_{ds}] indicates a modified, a-L DNA nucleotide with a 2′-deoxyribosyl sugar moiety, a phosphorothioate linkage, and base B.

For the results in the tables below, in vitro activity and toxicity experiments were performed essentially as described in Example 1. For in vitro activity and toxicity studies, 3T3-L1 cells were transfected with 27 nM, 80 nM, 250 nM, 740 nM, 2, 222 nM, 6,667 nM, or 20,000 nM of modified oligonucleotide by electroporation and levels of P21 and Gadd45a and Tnfrsf10b were measured by RT-qPCR as described in Example 1 above. The caspase assay was performed as described in Example 8 above in 3T3-L1 cells.

For the in vivo activity study in the tables below, 2 BALB/C mice per group were administered 1.8 mg/kg, 5.5 mg/kg, 16.7 mg/kg, 50 mg/kg, or 150 mg/kg doses of modified oligonucleotide, as indicated in the table below, by subcutaneous injection and sacrificed 72 hours later. For 558807, only 1.8 mg/kg, 5.5 mg/kg, and 16.7 mg/kg doses were tested for dose response, due to acute toxicity of higher doses. Liver mRNA was isolated an analyzed by RT-PCR as described in Example 1 above. Expression levels were normalized with Ribogreen® and are expressed relative to PBS-treated control mice.

TABLE 52
Activity and toxicity of modified oligonucleotides
complementary CXCL12

| | in vitro | in vitro | in vivo | ALT | ALT |
|---|---|---|---|---|---|
| | CXCL12 | RAPTOR | CXCL12 | @ 50 | @ 150 |
| Compound | IC₅₀ | IC₅₀ | ED₅₀ | mg/kg | mg/kg |
| ID | (μM) | (μM) | (mg/kg) | (IU/L) | (IU/L) |
| PBS | n/a | n/a | n/a | 25 @ 0 mg/kg | |
| 558807 | 0.10 | 1 | 2.9 | n.d.** | |
| 1215458 | 0.41 | >20 | 11 | 32 | 42 |
| 1215459 | 0.44 | >20 | 13 | 31 | 37 |
| 1215460 | 0.41 | >20 | 13 | 29 | 43 |
| 1215461 | 0.14 | 3 | 2.8 | 1725 | 6301 |
| 1215462 | 0.13 | 3 | 3.6 | 45 | 3652 |

**558807 treatment at 16.7 mg/kg leads to an ALT of 586 IU/L; mice that are treated with 558807 at 150 mg/kg typically experience death before 72 hours post-treatment.

TABLE 53
in vitro Caspase Activation

| Compound | 27 nM | 80 nM | 250 nM | 740 nM | 2,222 nM | 6,667 nM | 20,000 nM |
|---|---|---|---|---|---|---|---|
| ID | Relative Caspase Activation (% Control) | | | | | | |
| 558807 | 106 | 113 | 117 | 169 | 250 | 396 | 343 |
| 1215458 | 81 | 88 | 98 | 95 | 74 | 78 | 95 |
| 1215459 | 96 | 88 | 111 | 98 | 98 | 81 | 102 |
| 1215460 | 89 | 98 | 96 | 111 | 91 | 113 | 130 |
| 1215461 | 90 | 94 | 89 | 117 | 142 | 201 | 250 |
| 1215462 | 96 | 93 | 95 | 119 | 150 | 192 | 240 |

TABLE 53b
in vitro P21 Expression

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 558807 | 98 | 116 | 122 | 115 | 115 | 135 | 184 |
| 1215458 | 104 | 127 | 135 | 153 | 139 | 140 | 130 |
| 1215459 | 99 | 116 | 134 | 154 | 158 | 141 | 147 |
| 1215460 | 85 | 109 | 118 | 120 | 118 | 122 | 109 |
| 1215461 | 105 | 107 | 128 | 136 | 139 | 147 | 153 |
| 1215462 | 110 | 127 | 143 | 150 | 139 | 124 | 143 |

TABLE 53c
in vitro Tnfrsf10b Expression

| Compound | 27 nM | 80 nM | 250 nM | 740 nM | 2,222 nM | 6,667 nM | 20,000 nM |
|---|---|---|---|---|---|---|---|
| ID | Expression level of Tnfrsf10b mRNA (% Control) | | | | | | |
| 558807 | 107 | 108 | 105 | 99 | 113 | 102 | 68 |
| 1215458 | 90 | 88 | 92 | 87 | 81 | 78 | 80 |
| 1215459 | 97 | 108 | 108 | 100 | 103 | 94 | 83 |
| 1215460 | 92 | 100 | 99 | 102 | 95 | 95 | 84 |
| 1215461 | 86 | 91 | 99 | 98 | 97 | 97 | 114 |
| 1215462 | 101 | 97 | 98 | 56 | 82 | 101 | 108 |

TABLE 53d
in vitro Gadd45a Expression

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 558807 | 123 | 134 | 135 | 136 | 164 | 180 | 223 |
| 1215458 | 132 | 142 | 141 | 135 | 125 | 104 | 87 |
| 1215459 | 163 | 167 | 183 | 190 | 179 | 150 | 110 |
| 1215460 | 127 | 142 | 140 | 141 | 143 | 120 | 95 |
| 1215461 | 117 | 141 | 150 | 165 | 168 | 167 | 128 |
| 1215462 | 110 | 139 | 143 | 138 | 133 | 150 | 137 |

### Example 22 Stereochemical Isomers of Nucleosides

Modified oligonucleotides containing β-L-DNA nucleotides (described in Example 21 above) at various positions were synthesized. These modified oligonucleotides were compared to compound 558807, described in Table 1, Example 1 above. Compound 558807 contains 5-methyl cytosine for all cytosine nucleosides, as do compounds 1215458-1215460 described in the table below. The compounds in Table 54 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. Compounds 1244441-1244447 in the table below contain unmethylated cytosine in the central region of the compounds. The compounds in the table below are 100% complementary to mouse CXCL12, GENBANK NT_039353.7 truncated from 69430515 to 69445350 (SEQ ID NO: 1), at position 6877 to 6892.

TABLE 54
modified oligonucleotides with stereochemical modifications

| | position | | | |
|---|---|---|---|---|
| | of altered | stereochemical | | |
| | nucleotide | configuration | | SEQ |
| Compound | in central | of altered | | ID |
| ID | region | nucleotide | Chemistry Notation | NO |
| 1244441 | 1 | β-L-DNA | Gₖₛ^{m}CₖₛAₖₛ[_{β-L}T_{ds}]G_{ds}T_{ds}T_{ds}C_{ds}T_{ds}C_{ds}A_{ds}C_{ds}A_{ds}AₖₛTₖₛAₖ | 18 |
| 1215458 | 2 | β-L-DNA | Gₖₛ^{m}CₖₛAₖₛT_{ds} | 18 |
| | | | [_{β-L}G_{ds}]T_{ds}T_{ds}^{m}C_{ds}T_{ds}^{m}C_{ds}A_{ds}^{m}C_{ds}A_{ds}TₖₛTₖₛAₖ | |
| 1215459 | 3 | β-L-DNA | Gₖₛ^{m}CₖₛAₖₛT_{ds}G_{ds} | 18 |
| | | | [_{β-L}T_{ds}]T_{ds}^{m}C_{ds}T_{ds}^{m}C_{ds}A_{ds}^{m}C_{ds}A_{ds}TₖₛTₖₛAₖ | |
| 1215460 | 4 | β-L-DNA | Gₖₛ^{m}CₖₛAₖₛT_{ds}G_{ds}T_{ds} | 18 |
| | | | [_{β-L}T_{ds}]^{m}C_{ds}T_{ds}^{m}C_{ds}A_{ds}^{m}C_{ds}A_{ds}TₖₛTₖₛAₖ | |
| 1244442 | 5 | β-L-DNA | Gₖₛ^{m}CₖₛAₖₛT_{ds}G_{ds}T_{ds}T_{ds}[_{β-L}C_{ds}]T_{ds}C_{ds}A_{ds}C_{ds}A_{ds}TₖₛTₖₛAₖ | 18 |
| 1244443 | 6 | β-L-DNA | Gₖₛ^{m}CₖₛAₖₛT_{ds}G_{ds}T_{ds}T_{ds}C_{ds}[_{β-L}T_{ds}]C_{ds}A_{ds}C_{ds}A_{ds}TₖₛTₖₛAₖ | 18 |
| 1244444 | 7 | β-L-DNA | Gₖₛ^{m}CₖₛAₖₛT_{ds}G_{ds}T_{ds}T_{ds}C_{ds}T_{ds}[_{β-L}C_{ds}]A_{ds}C_{ds}A_{ds}TₖₛTₖₛAₖ | 18 |
| 1244445 | 8 | β-L-DNA | Gₖₛ^{m}CₖₛAₖₛT_{ds}G_{ds}T_{ds}T_{ds}C_{ds}T_{ds}C_{ds}[_{β-L}A_{ds}]C_{ds}A_{ds}TₖₛTₖₛAₖ | 18 |
| 1244446 | 9 | β-L-DNA | Gₖₛ^{m}CₖₛAₖₛT_{ds}G_{ds}T_{ds}T_{ds}C_{ds}T_{ds}C_{ds}A_{ds}[_{β-L}C_{ds}]A_{ds}TₖₛTₖₛAₖ | 18 |
| 1244447 | 10 | β-L-DNA | Gₖₛ^{m}CₖₛAₖₛT_{ds}G_{ds}T_{ds}T_{ds}C_{ds}T_{ds}C_{ds}A_{ds}C_{ds}[_{β-L}A_{ds}]TₖₛTₖₛAₖ | 18 |

A subscript “d” indicates a nucleoside comprising an n unmodified, 2′-β-D-deoxyribosyl sugar moiety. A subscript “k” indicates a cEt. A subscript “s” indicates a phosphorothioate internucleoside linkage. [_{β-L}B_{ds}] indicates a modified β-L-DNA nucleotide with a 2′-deoxyribosyl sugar moiety, a phosphorothioate linkage, and base B.

For the results in the tables below, in vitro activity and toxicity experiments were performed essentially as described in Example 1. For in vitro activity and toxicity studies, 3T3-L1 cells were transfected with 27 nM, 80 nM, 250 nM, 740 nM, 2, 222 nM, 6,667 nM, or 20,000 nM of modified oligonucleotide by electroporation and levels of P21 and Gadd45a and Tnfrsf10b were measured by RT-qPCR as described in Example 1 above. The caspase assay was performed as described in Example 8 above in 3T3-L1 cells. Localization of p54nrb in HeLa cells was quantitated as described in Example 15.

TABLE 55
In vitro activity and toxicity of modified
oligonucleotides complementary to CXCL12

| | in vitro | in vitro |
|---|---|---|
| | CXCL12 | Caspase |
| Compound | IC₅₀ | (% control) |
| ID | (nM) | @ 20 μM |
| 558807 | 0.029 | 321 |
| 1244441 | 0.471 | 108 |
| 1215458 | 0.200 | 104 |
| 1215459 | 0.191 | 111 |
| 1215460 | 0.130 | 133 |
| 1244442 | 0.134 | 185 |
| 1244443 | 0.083 | 279 |
| 1244444 | 0.109 | 213 |
| 1244445 | 0.198 | 249 |
| 1244446 | 0.127 | 243 |
| 1244447 | 0.080 | 333 |

### Example 23 Stereochemical Isomers of Nucleosides

Modified oligonucleotides containing α-D-DNA nucleotides (see below) at various positions were synthesized. These modified oligonucleotides were compared to the otherwise identical modified oligonucleotide lacking an altered nucleotide in the central region. The compounds in Table 54 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. The compounds in the table below are 100% complementary to mouse CXCL12, GENBANK NT_039353.7 truncated from 69430515 to 69445350 (SEQ ID NO: 1), at position 6877 to 6892.

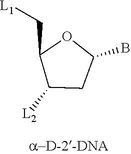

TABLE 56
modified oligonucleotides with stereochemical modifications

| | position | | | |
|---|---|---|---|---|
| | of altered | stereochemical | | |
| | nucleotide | configuration | | SEQ |
| Compound | in central | of altered | | ID |
| ID | region | nucleotide | Chemistry Notation | NO |
| 1244458 | none | none | Gₖₛ^{m}CₖₛAₖₛT_{ds}G_{ds}T_{ds}T_{ds}C_{ds}T_{ds}C_{ds}A_{ds}C_{ds}A_{ds}T_{ds}T_{ds}Aₖ | 18 |
| 1244448 | 1 | α-D-DNA | Gₖₛ^{m}CₖₛAₖₛ[_{α-D}T_{ds}]G_{ds}T_{ds}T_{ds}C_{ds}T_{ds}C_{ds}A_{ds}C_{ds}A_{ds}TₖₛTₖₛAₖ | 18 |
| 1244449 | 2 | α-D-DNA | Gₖₛ^{m}CₖₛAₖₛT_{ds}[_{α-D}G_{ds}]T_{ds}T_{ds}C_{ds}T_{ds}C_{ds}A_{ds}C_{ds}A_{ds}TₖₛTₖₛAₖ | 18 |
| 1244450 | 3 | α-D-DNA | Gₖₛ^{m}CₖₛAₖₛT_{ds}G_{ds}[_{α-D}T_{ds}]T_{ds}C_{ds}T_{ds}C_{ds}A_{ds}C_{ds}A_{ds}TₖₛTₖₛAₖ | 18 |
| 1244451 | 4 | α-D-DNA | Gₖₛ^{m}CₖₛAₖₛT_{ds}G_{ds}T_{ds}[_{α-D}T_{ds}]C_{ds}T_{ds}C_{ds}A_{ds}C_{ds}A_{ds}TₖₛTₖₛAₖ | 18 |
| 1244452 | 5 | α-D-DNA | Gₖₛ^{m}CₖₛAₖₛT_{ds}G_{ds}T_{ds}T_{ds}[_{α-D}C_{ds}]T_{ds}C_{ds}A_{ds}C_{ds}A_{ds}TₖₛTₖₛAₖ | 18 |
| 1244453 | 6 | α-D-DNA | Gₖₛ^{m}CₖₛAₖₛT_{ds}G_{ds}T_{ds}T_{ds}C_{ds}[_{α-D}T_{ds}]C_{ds}A_{ds}C_{ds}A_{ds}TₖₛTₖₛAₖ | 18 |
| 1244454 | 7 | α-D-DNA | Gₖₛ^{m}CₖₛAₖₛT_{ds}G_{ds}T_{ds}T_{ds}C_{ds}T_{ds}[_{α-D}C_{ds}]A_{ds}C_{ds}A_{ds}TₖₛTₖₛAₖ | 18 |
| 1244455 | 8 | α-D-DNA | Gₖₛ^{m}CₖₛAₖₛT_{ds}G_{ds}T_{ds}T_{ds}C_{ds}T_{ds}C_{ds}[_{α-D}A_{ds}]C_{ds}A_{ds}TₖₛTₖₛAₖ | 18 |
| 1244456 | 9 | α-D-DNA | Gₖₛ^{m}CₖₛAₖₛT_{ds}G_{ds}T_{ds}T_{ds}C_{ds}T_{ds}C_{ds}A_{ds}[_{α-D}C_{ds}]A_{ds}TₖₛTₖₛAₖ | 18 |
| 1244457 | 10 | α-D-DNA | Gₖₛ^{m}CₖₛAₖₛT_{ds}G_{ds}T_{ds}T_{ds}C_{ds}T_{ds}C_{ds}A_{ds}C_{ds}[_{α-D}C_{ds}]TₖₛTₖₛAₖ | 18 |

A subscript “d” indicates a nucleoside comprising an n unmodified, 2′-β-D-deoxyribosyl sugar moiety. A subscript “k” indicates a cEt. A subscript “s” indicates a phosphorothioate internucleoside linkage. [_{α-D-}B_{ds}] indicates a modified, α-D-DNA nucleotide with a 2′-deoxyribosyl sugar moiety, a phosphorothioate linkage, and base B.

For the results in the tables below, in vitro activity and toxicity experiments were performed essentially as described in Example 1. For in vitro activity and toxicity studies, 3T3-L1 cells were transfected with 27 nM, 80 nM, 250 nM, 740 nM, 2, 222 nM, 6,667 nM, or 20,000 nM of modified oligonucleotide by electroporation and levels of p21 were measured by RT-qPCR as described in Example 1 above. The caspase assay was performed as described in Example 8 above in 3T3-L1 cells. Localization of p54nrb in HeLa cells was quantitated as described in Example 15.

TABLE 57
In vitro activity and toxicity of modified
oligonucleotides complementary CXCL12

| | in vitro | in vitro | in vitro | |
|---|---|---|---|---|
| | CXCL12 | Caspase | p21 | % |
| Compound | IC₅₀ | (% control) | (% control) | nucleolar |
| ID | (nM) | @ 20 μM | @ 20 μM | p54nrb |
| 1244458 | 19 | 785 | 327 | 86 |
| 1244448 | 35 | 269 | 135 | 66 |
| 1244449 | 169 | 111 | 101 | 8 |
| 1244450 | 103 | 96 | 169 | 11 |
| 1244451 | 45 | 261 | 206 | 78 |
| 1244452 | 393 | 295 | 146 | 83 |
| 1244453 | 80 | 417 | 255 | 92 |
| 1244454 | 512 | 287 | 240 | 65 |
| 1244455 | 125 | 409 | 310 | 83 |
| 1244456 | 247 | 233 | 269 | 96 |
| 1244457 | 31 | 854 | 400 | 100 |

### Example 24 4′-Methyl and Xylo DNA

Modified oligonucleotides containing an altered nucleotide with a 4′-methyl modified sugar moiety or a 2′-deoxy-β-D-xylofuranosyl (2′deoxy-β-D-XNA) sugar moiety at various positions were synthesized (see Table 58 below). Synthesis of oligonucleotides comprising 2′deoxy-β-D-XNA nucleosides has been described previously (Wang, et. al., Biochemistry, 56(29): 3725-3732, 2017). Synthesis of oligonucleotides comprising 4′-methyl modified nucleosides has been described previously (e.g., Detmer et. al., European J. Org. Chem, 1837-1846, 2003).. The compounds in Table 58 each comprise a 5′ wing and a 3′ wing each consisting of three linked cEt nucleosides and a central region comprising nucleosides each comprising 2′-β-D-deoxyribosyl sugar moieties aside from the altered nucleotide, as indicated. Each internucleoside linkage is a phosphodiester internucleoside linkage. These compounds were compared to a compound comprising a 2′-OMe modified sugar moiety at position 2 of the central region, 936053, described in Example 1 above. The compounds in the table below are 100% complementary to mouse CXCL12, GENBANK NT_039353.7 truncated from 69430515 to 69445350 (SEQ ID NO: 1), at position 6877 to 6892.

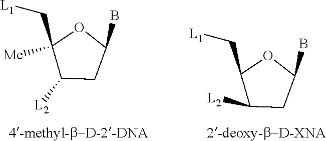

TABLE 58
modified oligonucleotides with stereochemical modifications

| | position | | | |
|---|---|---|---|---|
| | of altered | | | |
| | nucleotide | modification | | SEQ |
| Compound | in central | of altered | | ID |
| ID | region | nucleotide | Chemistry Notation | NO |
| 936053 | 2 | 2′-OMe | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | 18 |
| 1244461 | 3 | 4′-Me | Gₖₛ^{m}CₖₛAₖₛT_{ds}G_{ds}T_{[4m]s}T_{ds}^{m}C_{ds}T_{ds}^{m}C_{ds}A_{ds}^{m}C_{ds}A_{ds}TₖₛTₖₛAₖ | 18 |
| 1244462 | 4 | 4′-Me | Gₖₛ^{m}CₖₛAₖₛT_{ds}G_{ds}T_{ds}T_{[4m]s}^{m}C_{ds}T_{ds}^{m}C_{ds}A_{ds}^{m}C_{ds}A_{ds}TₖₛTₖₛAₖ | 18 |
| 1263776 | 3 | β-D-XNA | Gₖₛ^{m}CₖₛAₖₛT_{ds}G_{ds}[_{β-D}Tₓₛ]T_{ds}^{m}C_{ds}T_{ds}^{m}C_{ds}A_{ds}^{m}C_{ds}A_{ds}TₖₛTₖₛAₖ | 18 |
| 1263777 | 4 | β-D-XNA | Gₖₛ^{m}CₖₛAₖₛT_{ds}G_{ds}T_{ds}[_{β-D}Tₓₛ]^{m}C_{ds}T_{ds}^{m}C_{ds}A_{ds}^{m}C_{ds}A_{ds}TₖₛTₖₛAₖ | 18 |

A subscript “d” indicates an unmodified, 2′-β-D-deoxyribosyl sugar moiety. A subscript “k” indicates a cEt.A subscript “s” indicates a phosphorothioate internucleoside linkage. A superscript “m” indicates 5-methyl Cytosine. A subscript “[4m]” indicates a 4′-methyl-2′-β-D-deoxyribosyl sugar moiety. [_{β-D-}Bₓₛ] indicates a modified, β-D-XNA (xylo) nucleotide with a 2′-deoxyxylosyl sugar moiety, a phosphorothioate linkage, and base B.

For in vivo activity and toxicity studies, 3 BALB/c mice per group were administered 10 or 150 mg/kg modified oligonucleotide by subcutaneous injection and sacrificed after 72 hours. Four animals were administered saline to serve as a control. RT-PCR was performed as described in Example 1 to determine mRNA levels of CXCL12, P21, Tnfrsf10b, and Gadd45a. Plasma levels of ALT was measured using an automated clinical chemistry analyzer. Increased ALT is indicative of acute liver toxicity.

TABLE 59
In vivo activity and toxicity of modified oligonucleotides complementary to CXCL12

| | in vivo | in vivo | in vivo | in vivo | in vivo | in vivo | in vivo |
|---|---|---|---|---|---|---|---|
| | CXCL12 | CXCL12 | P21 | Tnfrsf10b | Gadd45a | ALT | ALT |
| | @ 10 | @ 150 | @ 150 | @ 150 | @ 150 | @ 10 | @ 150 |
| Compound | mg/kg | mg/kg | mg/kg | mg/kg | mg/kg | mg/kg | mg/kg |
| ID | (% control) | (% control) | (% control) | (% control) | (% control) | (IU/L) | (IU/L) |
| PBS | 100 | 100 | 100 | 100 | 100 | 26 (@ 0 mg/kg) | |
| 936053 | 37 | 13 | 175 | 448 | 216 | 23 | 83 |
| 1244461 | 22 | 5 | 2994 | 4663 | 1124 | 31 | 5080 |
| 1244462 | 30 | 7* | 1038 | 717* | 407* | 28 | 1789* |
| 1263776 | 19 | 11 | 4846 | 10686 | 1032 | 27 | 9234 |
| 1263777 | 13 | n.d. | n.d. | n.d. | n.d. | 58 | death |

*Value represents the average of 2 samples.

### Example 25 Microscopy

Selected modified nucleotides described in the Examples above were tested for their effect on HeLa cells by microscopy. HeLa cells were transfected by lipofectamine 2000 with 200 nM of modified oligonucleotide for 2 hrs and then cellular protein p54nrb was stained by mP54 antibody (Santa Cruz Biotech, sc-376865) and DAPI was used to stain for the nucleus of cells.

For experiments reported in the tables below, the number of cells with nucleolar p54nrb and the total number of cells in the images were counted and a percentage of cells with mislocalization of p54nrb was calculated. Where the same compound appears in multiple tables, these represent the results from independent experiments.

TABLE 60
Nucleolar mislocalization of p54nrb

| Compound | Cells with | Total | % cells with |
|---|---|---|---|
| ID | nucleolar p54nrb | cells | mislocalization |
| Mock | 0 | 74 | 0 |
| 558807 | 45 | 51 | 88 |

TABLE 61
Nucleolar mislocalization of p54nrb

| | | | ALT @ |
|---|---|---|---|
| Compound | | % cells with | 150 mg/kg |
| ID | Sugar Motif | mislocalization | (IU/L) |
| 464917 | kkk-d(10)-kkk | 75 | death |
| 982034 | kkk-d-m-(8)-kkk | <7 | 13,172 |
| 465175 | kkk-d(10)-kkk | 57 | 1,564 |
| 1133329 | kkk-d-m-(8)-kkk | 48 | 60 |
| 465181 | kkk-d(10)-kkk | 58 | 7,087 |
| 1133335 | kkk-d-m-(8)-kkk | <1 | 26 |
| 545984 | kkk-d(10)-kkk | 98 | 37,277 |
| 1133060 | kkk-d-m-(8)-kkk | 0 | 39 |

For experiments reported in the tables below, selected images were qualitatively analyzed and assigned a score of (−), indicating that no p54nrb mislocalization to the nucleolus was observed, or a score of (+) indicating that mislocalization of p54nrb to the nucleolus was observed in some cells, or (++) indicating that p54nrb mislocalizization to the nucleolus was observed in most or all cells.

TABLE 62
Nucleolar mislocalization of p54nrb and correlation with toxicity

| Compound | Complementary | Mislocalization | ALT @ max |
|---|---|---|---|
| ID | mRNA | of p54nrb | dose* |
| 558807 | CXCL12 | ++ | death |
| 936049 | CXCL12 | ++ | 8,622 |
| 936053 | CXCL12 | − | 46 |
| 892826 | CXCL12 | − | 10,463 |
| 766677 | CXCL12 | − | 23 |
| 828911 | CXCL12 | − | 27 |
| 936051 | CXCL12 | + | death |
| 936052 | CXCL12 | − | 1,110 |
| 1070041 | CXCL12 | + | 96 |
| 1061314 | CXCL12 | − | 26 |
| 1061302 | CXCL12 | + | 2,253 |
| 1061303 | CXCL12 | − | 34 |
| 1061304 | CXCL12 | − | 52 |
| 1061305 | CXCL12 | − | 31 |
| 1076587 | CXCL12 | − | n.d. |
| 1076588 | CXCL12 | + | n.d. |
| 1069852 | CXCL12 | − | n.d. |
| 1061328 | CXCL12 | + | n.d. |
| 1061955 | CXCL12 | − | 86 |
| 1061964 | CXCL12 | − | n.d. |
| 1244441 | CXCL12 | − | n.d. |
| 1215458 | CXCL12 | − | n.d. |
| 1215459 | CXCL12 | − | n.d. |
| 1215460 | CXCL12 | − | n.d. |
| 1244442 | CXCL12 | − | n.d. |
| 1244443 | CXCL12 | + | n.d. |
| 1244444 | CXCL12 | ++ | n.d. |
| 1244445 | CXCL12 | ++ | n.d. |
| 1244446 | CXCL12 | ++ | n.d. |
| 1244447 | CXCL12 | ++ | n.d. |
| 464917 | FXI | + | 18,316 |
| 465977 | FXI | + | death |
| 483706 | FXI | + | 1,424 |
| 443919 | FXI | − | 68 |
| 820685** | FXI | − | 59 |
| 508031 | SOD1 | ++ | 16,317 |
| 895154 | SOD1 | + | 206 |
| 895155 | SOD1 | − | 41 |
| 895156 | SOD1 | + | 1,242 |
| 508034 | SOD1 | + | 22,396 |
| 508037 | SOD1 | − | 20 |
| 529933 | SOD1 | − | 11 |

*Data presented in previous examples; maximum administered dose is 150 mg/kg for modified oligonucleotides complementary to CXCL12, 100 mg/kg for modified oligonucleotides complementary to SOD1, and 33 mg/kg for compounds complementary to FXI, except that the ALT for 820685 is at 100 mg/kg.
**820685 has the same sequence as 464917 and a sugar motif of kkk-m(10)-kkk.

### Example 26 Nucleolar Mislocalization of p54Nrb with Fluorescently-Labeled Modified Oligonucleotides

Modified oligonucleotides described in the tables above were conjugated to Cy3 or FAM on the 3′-end via phosphorothioate linker or on the 5′-end via a phosphorothioate linker to generate a compound comprising a conjugate group that comprises a fluorophore, resulting in a fluorescently labeled modified oligonucleotide. Fluorescently labeled modified oligonucleotides were incubated with HeLa cells at 200 nM for 2 hours and cells were imaged by fluorescent microscopy. Cellular protein p54nrb was stained by mP54 antibody (Santa Cruz Biotech, sc-376865) and DAPI was used to stain for the nucleus of cells. For experiments reported in the tables below, images were qualitatively analyzed and assigned a score of (−), indicating that no p4nrb mislocalization to the nucleolus was observed, or a score of (+) indicating that mislocalization of p4nrb to the nucleolus was observed in some cells, or (++) indicating that p54nrb mislocalization to the nucleolus was observed in most or all cells.

TABLE 63
Fluorescently Labeled Modified Oligonucleotides

| Unlabelled | Fluorescently- | | SEQ |
|---|---|---|---|
| compound | labelled | Chemistry notation | ID |
| ID | compound ID | for Fluorescently-labelled compound | NO: |
| 558807 | 925819 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖₛ-Cy3 | 18 |
| 766676 | 925820 | Gₖₛ^{m}CₖₛAₖₛT_{dx}G_{ds}T_{ds}T_{ds}^{m}C_{ds}T_{ds}^{m}C_{ds}A_{ds}^{m}C_{ds}A_{ds}TₖₛTₖₛAₖₛ-Cy3 | 18 |
| 766677 | 925821 | Gₖₛ^{m}CₖₛAₖₛT_{ds}G_{dx}T_{ds}T_{ds}^{m}C_{ds}T_{ds}^{m}C_{ds}A_{ds}^{m}C_{ds}A_{ds}TₖₛTₖₛAₖₛ-Cy3 | 18 |
| 766678 | 925822 | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖₛ-Cy3 | 18 |
| 766679 | 925826 | Gₖₛ^{m}CₖₛAₖₛT_{ds}G_{ds}T_{ds}T_{dx}^{m}C_{ds}T_{ds}^{m}C_{ds}A_{ds}^{m}C_{ds}A_{ds}TₖₛTₖₛAₖₛ-Cy3 | 18 |
| 766684 | 925824 | Gₖₛ^{m}CₖₛAₖₛT_{ds}G_{ds}T_{ds}T_{ds}^{m}C_{ds}T_{ds}^{m}C_{ds}A_{ds}^{m}C_{dx}A_{ds}TₖₛTₖₛAₖₛ-Cy3 | 18 |
| 936049 | 958339 | Cy3-Gₖₛ^{m}CₖₛAₖₛUₘₛG_{ds}T_{ds}T_{ds}^{m}C_{ds}T_{ds}^{m}C_{ds}A_{ds}^{m}C_{ds}A_{ds}TₖₛTₖₛAₖ | 19 |
| 936053 | 958340 | Cy3-Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | 18 |
| 892826 | 958341 | Cy3-Gₖₛ^{m}CₖₛAₖₛT_{ds}G_{ds}UₘₛT_{ds}^{m}C_{ds}T_{ds}^{m}C_{ds}A_{ds}^{m}C_{ds}A_{ds}TₖₛTₖₛAₖ | 20 |
| 558807 | 1189295 | FAM-Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖₛ | 18 |
| 1061955 | 1189310 | FAM-Gₖₛ^{m}CₖₛAₖₛT_{ds}I_{ds}T_{ds}T_{ds}^{m}C_{ds}T_{ds}^{m}C_{ds}A_{ds}^{m}C_{ds}A_{ds}TₖₛTₖₛAₖₛ | 30 |
| 766677 | 1215929 | FAM-Gₖₛ^{m}CₖₛAₖₛT_{ds}G_{dx}T_{ds}T_{ds}^{m}C_{ds}T_{ds}^{m}C_{ds}A_{ds}^{m}C_{ds}A_{ds}TₖₛTₖₛAₖₛ | 18 |
| 936053 | 1189369 | FAM-Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | 18 |
| 942944 | 1215928 | FAM-Gₖₛ^{m}CₖₛAₖₛT_{ds}G_{ds}T_{ds}^{(R)-m}T_{ds}^{m}C_{ds}T_{ds}^{m}C_{ds}A_{ds}^{m}C_{ds}A_{ds}TₖₛTₖₛAₖ | 18 |
| 464917 | 813223 | Cy3-GₖₛTₖₛ^{m}CₖₛT_{ds}G_{ds}T_{ds}G_{ds}^{m}C_{ds}A_{ds}T_{ds}^{m}C_{ds}T_{ds}^{m}C_{ds}Tₖₛ^{m}Cₖₛ^{m}Cₖ | 22 |
| 508031 | 828939 | Cy3-TₖₛGₖₛAₖₛG_{ds}G_{ds}T_{ds}^{m}C_{ds}^{m}C_{ds}T_{ds}G_{ds}^{m}C_{ds}A_{ds}^{m}C_{ds}TₖₛGₖₛGₖ | 26 |
| 482050 | 841864 | Cy3-AₖₛTₖₛ^{m}CₖₛA_{ds}T_{ds}G_{ds}G_{ds}^{m}C_{ds}T_{ds}G_{ds}^{m}C_{ds}A_{ds}G_{ds}^{m}CₖₛTₖₛTₖ | 24 |
| 449093 | 489982 | FAM-TₖₛTₖₛ^{m}CₖₛA_{ds}G_{ds}T_{ds}^{m}C_{ds}A_{ds}T_{ds}G_{ds}A_{ds}^{m}C_{ds}T_{ds}Tₖₛ^{m}Cₖₛ^{m}Cₖ | 28 |
| 465178 | 869208 | Cy3-GₖₛTₖₛ^{m}CₖₛA_{ds}G_{ds}T_{ds}A_{ds}T_{ds}^{m}C_{ds}^{m}C_{ds}^{m}C_{ds}A_{ds}G_{ds}TₖₛGₖₛTₖ | 89 |
| 575013 | 869198 | Cy3-^{m}CₖₛAₖₛGₖₛA_{ds}T_{ds}A_{ds}T_{ds}A_{ds}G_{ds}G_{ds}A_{ds}^{m}C_{ds}T_{ds}GₖₛGₖₛAₖ | 110 |
| 549139 | 869199 | Cy3-GₖₛAₖₛ^{m}CₖₛG_{ds}^{m}C_{ds}G_{ds}^{m}C_{ds}^{m}C_{ds}T_{ds}G_{ds}A_{ds}A_{ds}G_{ds}GₖₛTₖₛTₖ | 111 |
| 508032 | 869200 | Cy3-GₖₛGₖₛAₖₛ^{m}C_{ds}A_{ds}^{m}C_{ds}A_{ds}T_{ds}T_{ds}G_{ds}G_{ds}^{m}C_{ds}^{m}C_{ds}Aₖₛ^{m}CₖₛAₖ | 112 |
| 464932 | 869201 | Cy3-GₖₛGₖₛ^{m}Cₖₛ^{m}C_{ds}A_{ds}^{m}C_{ds}^{m}C_{ds}A_{ds}^{m}C_{ds}G_{ds}^{m}C_{ds}T_{ds}G_{ds}Tₖₛ^{m}CₖₛAₖ | 113 |
| 465131 | 869202 | Cy3-TₖₛGₖₛ^{m}Cₖₛ^{m}C_{ds}A_{ds}^{m}C_{ds}^{m}C_{ds}G_{ds}T_{ds}A_{ds}G_{ds}A_{ds}^{m}C_{ds}Aₖₛ^{m}CₖₛGₖ | 114 |
| 147420 | 841863 | Cy3-AₑₛAₑₛTₑₛGₑₛTₑₛG_{ds}^{m}C_{ds}^{m}C_{ds}T_{ds}G_{ds}^{m}C_{ds}T_{ds}G_{ds}T_{ds}^{m}C_{ds}^{m}CₑₛTₑₛTₑₛGₑₛAₑ | 73 |

A subscript “d” indicates an unmodified, 2′-β-D-deoxyribosyl sugar moiety. A subscript “e” indicates a 2′-MOE-β-D-ribofuranosyl sugar moiety. A subscript “m” indicates a 2′-O-methyl-β-D-ribofuranosyl sugar moiety. A subscript “k” indicates a cEt. A subscript “s” indicates a phosphorothioate internucleoside linkage. A superscript “m” before a C indicates 5-methyl Cytosine.. A subscript “x” indicates a MOP internucleoside linkage.

TABLE 64
Nucleolar mislocalization of p54nrb

| | | | | ALT @ |
|---|---|---|---|---|
| | | | | 150 mg/kg of |
| Unlabelled | Fluorescently- | Comple- | Mislocal- | unlabelled |
| compound | labelled | mentary | ization | modified |
| ID | Compound ID | mRNA | of p54nrb | oligonucleotide |
| 558807 | 925819 | CXCL12 | ++ | death |
| 766676 | 925820 | CXCL12 | ++ | 5,475 |
| 766677 | 925821 | CXCL12 | − | 23 |
| 766678 | 925822 | CXCL12 | − | 67 |
| 766679 | 925823 | CXCL12 | + | 3,347 |
| 766684 | 925824 | CXCL12 | ++ | death |
| 936049 | 958339 | CXCL12 | ++ | 8,622 |
| 936053 | 958340 | CXCL12 | − | 46 |
| 892826 | 958341 | CXCL12 | + | 10,463 |
| 558807 | 1189295 | CXCL12 | ++ | death |
| 1061955 | 1189310 | CXCL12 | − | 86 |
| 766677 | 1215929 | CXCL12 | − | 23 |
| 936053 | 1189369 | CXCL12 | − | 46 |
| 942944 | 1215928 | CXCL12 | + | 233 |

TABLE 65
Nucleolar mislocalization of p54nrb

| | | | | ALT @ |
|---|---|---|---|---|
| | | | | 200 mg/kg of |
| | Fluorescently- | Comple- | Mislocal- | unlabelled |
| Unlabelled | labelled | mentary | ization | modified |
| compound ID | Compound ID | mRNA | of p54nrb | oligonucleotide |
| 147420 | 841863 | CPT1A | ++ | 7,794 |

For in vivo maximum tolerated doses reported in the table below, 2-4 BALB/C mice per group were administered modified oligonucleotide at 3.7, 11, 33, 100, or 300 mg/kg by subcutaneous injection and sacrificed after 72 hours. Maximum tolerated dose is the highest dose at which ALT is below 5× that in PBS-treated control mice, or ˜150 IU/L.

TABLE 67
Nucleolar mislocalization of p54nrb

| | | | | Maximum |
|---|---|---|---|---|
| | | | Mislocal- | tolerated |
| | | | ization of | dose un- |
| | | | p54nrb, | labelled |
| | | | labelled | modified |
| | Fluorescently- | Comple- | modified | oligo- |
| Unlabelled | labelled | mentary | oligo- | nucleotide |
| compound ID | compound ID | mRNA | nucleotide | (mg/kg, mouse) |
| 464917 | 813223 | FXI | + | 11 |
| 508031 | 828939 | SOD1 | ++ | 33 |
| 482050 | 841864 | PTEN | ++ | 33 |
| 449093 | 489982 | SRB1 | ++ | 33 |
| 465178 | 869208 | FXI | + | 100 |
| 575013 | 869198 | FXII | − | >300 |
| 549139 | 869199 | none | − | >300 |
| 508032 | 869200 | SOD1 | − | >300 |
| 464932 | 869201 | FXI | − | >300 |
| 465131 | 869202 | FXI | − | >300 |

TABLE 68
Nucleolar mislocalization of p54nrb

| | Fluorescently- | Cells with | | % cells |
|---|---|---|---|---|
| Unlabelled | labelled | nucleolar | Total | with p54nrb |
| compound ID | compound ID | p54nrb | cells | mislocalization |
| 558807 | 925819 | 57 | 74 | 77 |
| 936049 | 958339 | 51 | 72 | 71 |
| 936053 | 958340 | 6 | 65 | 9 |
| 892826 | 958341 | 30 | 53 | 57 |

### Example 27 In Vivo and In Vitro Toxicity of LNA-Containing Modified Oligonucleotides

Modified oligonucleotides in the table below have a 3-10-3 sugar motif with LNA nucleosides on the 5′ and 3′ ends and DNA nucleosides in the central region.

TABLE 69
Modified Oligonucleotides

| Compound | | SEQ |
|---|---|---|
| ID | Chemistry notation | ID NO: |
| 569713 | GₗₛAₗₛ^{m}CₗₛG_{ds}^{m}C_{ds}G_{ds}^{m}C_{ds}^{m}C_{ds}T_{ds}G_{ds}A_{ds}A_{ds}G_{ds}GₗₛTₗₛTₗ | 111 |
| 569717 | AₗₛTₗₛ^{m}CₗₛA_{ds}T_{ds}G_{ds}G_{ds}^{m}C_{ds}T_{ds}G_{ds}^{m}C_{ds}A_{ds}G_{ds}^{m}CₗₛTₗₛTₗ | 24 |
| 569719 | GₗₛTₗₛ^{m}CₗₛT_{ds}G_{ds}T_{ds}G_{ds}^{m}C_{ds}A_{ds}T_{ds}^{m}C_{ds}T_{ds}^{m}C_{ds}Tₗₛ^{m}Cₗₛ^{m}Cₗ | 22 |
| 569721 | TₗₛGₗₛAₗₛG_{ds}G_{ds}T_{ds}^{m}C_{ds}^{m}C_{ds}T_{ds}G_{ds}^{m}C_{ds}A_{ds}^{m}C_{ds}TₗₛGₗₛGₗ | 26 |
| 814336 | Gₗₛ^{m}CₗₛAₗₛT_{ds}G_{ds}T_{ds}T_{ds}^{m}C_{ds}T_{ds}^{m}C_{ds}A_{ds}^{m}C_{ds}A_{ds}TₗₛTₗₛAₗ | 18 |

A subscript “d” indicates an unmodified, 2′-β-D-deoxyribosyl sugar moiety. A subscript “l” indicates a β-D-LNA sugar moiety. A subscript “s” indicates a phosphorothioate internucleoside linkage. A superscript “m” before a C indicates 5-methyl Cytosine.

Modified nucleotides with 3-10-3 lll-d(10)-lll sugar motifs were tested for their effect on 3T3 cells by microscopy. 3T3 cells were transfected by lipofectamine 2000 with 200 nM of modified oligonucleotide for 2 hrs and then cellular protein p54nrb was stained by mP54 antibody (Santa Cruz Biotech, sc-376865) and DAPI was used to stain for the nucleus of cells. For experiments reported in the tables below, images were qualitatively analyzed and assigned a score of (−), indicating that no p54nrb mislocalization to the nucleolus was observed, or a scale of (+) indicating that mislocalization of p54nrb to the nucleolus was observed in some cells, or (++) indicating that p54nrb mislocalization to the nucleolus was observed in most or all cells. Modified nucleotides with 3-10-3 lll-d(10)-lll or kkk-d(10)-kkk sugar motifs were tested in vivo. For in vivo toxicity data, 2-4 BALB/C mice were administered modified oligonucleotide by subcutaneous injection at the doses indicated in the table below. Mice were sacrificed after 72 hours and mRNA was isolated and analyzed as described in Example 1 above. ALT values in plasma were obtained using a clinical chemistry analyzer.

TABLE 70
Modified oligonucleotide dosages administered to mice

| | | Dose 2 (mg/kg) |
|---|---|---|
| | | (Maximum |
| Compound ID | Dose 1 (mg/kg) | dose) |
| 549139 | 300 | n/a |
| 569713 | 300 | n/a |
| 482050 | 33 | 100 |
| 569717 | 33 | 100 |
| 464917 | 11 | 33 |
| 569719 | 11 | 33 |
| 508031 | 33 | 100 |
| 569721 | 33 | 100 |
| 558807 | 17 | 51 |
| 814336 | 17 | 51 |

TABLE 71
In vitro p54nrb localization and in vitro toxicity

| | | | | P21 | Tnfrsf10b |
|---|---|---|---|---|---|
| | | | | mRNA at | mRNA at |
| Com- | Comple- | Mislocal- | ALT @ | maximum | maximum |
| pound | mentary | ization | maximum | dose (% | dose (% |
| ID | mRNA | of p54nrb | dose | control) | control) |
| 549139 | none | − | 35 | 306 | 252 |
| 569713 | none | − | 44 | 449 | 241 |
| 482050 | PTEN | n.d. | 6555 | 10,430 | 4,232 |
| 569717 | PTEN | n.d. | 270 | 17,295 | 9,568 |
| 464917 | FXI | ++ | 13,920 | 9,590 | 7,731 |
| 569719 | FXI | + | 14,449 | 13,020 | 6,569 |
| 508031 | SOD1 | ++ | 18,550 | 8,909 | 6,678 |
| 569721 | SOD1 | + | 33,246 | 12,193 | 9,169 |
| 558807 | CXCL12 | ++ | 9,510 | 11,904 | 6,831 |
| 814336 | CXCL12 | ++ | death* | n.d. | n.d. |

*At 17 mg/kg, ALT was 4725, P21 mRNA was 11,567, and Tnfrsf10b mRNA was 8,636.

For the in vitro study reported in the tables below, 3T3-L1 cells were electroporated with 27 nM, 80 nM, 250 nM, 740 nM, 2, 222 nM, 6,667 nM, or 20,000 nM of modified oligonucleotide and levels of P21 and Gadd45a were measured by RT-qPCR as described in Example 1 above. Caspase-3 and caspase-7 activation was measured using the Caspase-Glo® 3/7 Assay System (G8090, Promega). Levels of caspase activation correlate with apoptotic cell death.

TABLE 72
in vitro Caspase Activation

| Compound | 27 nM | 80 nM | 250 nM | 740 nM | 2,222 nM | 6,667 nM | 20,000 nM |
|---|---|---|---|---|---|---|---|
| ID | Caspase Activation | | | | | | |
| 549139 | 2693 | 2272 | 2536 | 2170 | 2664 | 2128 | 2406 |
| 569713 | 2219 | 1988 | 1996 | 1892 | 2099 | 2178 | 3202 |
| 464917 | 1988 | 2116 | 1907 | 2365 | 6580 | 13268 | 24228 |
| 569719 | 2080 | 2183 | 2610 | 4225 | 10773 | 14199 | 20524 |
| 508031 | 7082 | 6602 | 7123 | 8876 | 14962 | 20060 | 29955 |
| 569721 | 7905 | 7741 | 8508 | 10364 | 20715 | 24370 | 49476 |
| 558807 | 7272 | 7887 | 8672 | 12555 | 19397 | 25124 | 28133 |
| 814336 | 7308 | 7975 | 9150 | 12927 | 21327 | 26992 | 26794 |

TABLE 73
in vitro P21 Expression

| Compound | 27 nM | 80 nM | 250 nM | 740 nM | 2,222 nM | 6,667 nM | 20,000 nM |
|---|---|---|---|---|---|---|---|
| ID | Expression level of P21 mRNA (% Control) | | | | | | |
| 549139 | 108 | 104 | 93 | 97 | 97 | 88 | 99 |
| 569713 | 116 | 105 | 94 | 127 | 129 | 139 | 166 |
| 464917 | 129 | 132 | 145 | 149 | 275 | 595 | 1044 |
| 569719 | 120 | 118 | 144 | 160 | 332 | 731 | 922 |
| 508031 | 100 | 90 | 99 | 102 | 100 | 124 | 247 |
| 569721 | 116 | 104 | 123 | 119 | 148 | 123 | 470 |
| 558807 | 95 | 126 | 123 | 123 | 104 | 119 | 193 |
| 814336 | 86 | 100 | 96 | 85 | 119 | 170 | 254 |

TABLE 74
in vitro Gadd45a Expression

| Compound | 27 nM | 80 nM | 250 nM | 740 nM | 2,222 nM | 6,667 nM | 20,000 nM |
|---|---|---|---|---|---|---|---|
| ID | Expression level of Gadd45a mRNA (% Control) | | | | | | |
| 549139 | 113 | 125 | 105 | 83 | 72 | 61 | 35 |
| 569713 | 168 | 139 | 116 | 154 | 135 | 162 | 147 |
| 464917 | 153 | 170 | 187 | 210 | 376 | 906 | 933 |
| 569719 | 165 | 168 | 217 | 220 | 514 | 1223 | 1086 |
| 508031 | 106 | 115 | 111 | 112 | 114 | 211 | 345 |
| 569721 | 165 | 168 | 158 | 136 | 212 | 326 | 451 |
| 558807 | 200 | 198 | 222 | 216 | 200 | 235 | 263 |
| 814336 | 117 | 113 | 139 | 148 | 169 | 198 | 278 |

TABLE 75
in vitro Tnfrsf10b Expression

| Compound | 27 nM | 80 nM | 250 nM | 740 nM | 2,222 nM | 6,667 nM | 20,000 nM |
|---|---|---|---|---|---|---|---|
| ID | Expression level of Tnfrsf10b mRNA (% Control) | | | | | | |
| 549139 | 93 | 96 | 87 | 87 | 89 | 98 | 96 |
| 569713 | 116 | 111 | 79 | 119 | 115 | 128 | 114 |
| 464917 | 122 | 127 | 129 | 93 | 116 | 186 | 125 |
| 569719 | 105 | 107 | 117 | 88 | 119 | 151 | 36 |

### Example 28 Total Protein Binding of Modified Oligonucleotides Complementary to SOD1

Modified oligonucleotides described in the examples above were evaluated for their total protein binding in HeLa nuclear lysate. Cellular proteins were captured with the biotin-conjugated modified oligonucleotide 791136, which has a 5′-biotin-TEG, the sugar motif kkk-d(10)-kkk and the same sequence as 464917, GTCTGTGCATCTCTCC (SEQ ID NO: 22) and eluted with increasing concentrations of 508031, 895154, 895155, and 895156, described in Example 6 above. Eluted proteins were run on an SDS-PAGE gel. Increased total protein binding is observed for compound 508031 and 895154 compared to compound 895155 and 895156.

### Example 29 Total Protein Binding of Modified Oligonucleotides Complementary to FXI

Modified oligonucleotides described in the examples above were evaluated for their total protein binding in HeLa nuclear lysate. Cellular proteins were captured with the biotin-conjugated modified oligonucleotide 791136, which has a 5′-biotin-TEG, the sugar motif kkk-d(10)-kkk and the same sequence as 464917, GTCTGTGCATCTCTCC (SEQ ID NO: 22) and eluted with increasing concentrations of 464917, 465977, 483706, and 820685, described in Examples 4 and 25 above. Eluted proteins were run on an SDS-PAGE gel. Increased total protein binding is observed for compound 464917 and 465977 compared to compounds 483706 and 820685. A series of western blots was done to detect SSBP1, NCL1, PCNA, p54nrb, RNase H1, and PSF.

In an independent experiment, cellular proteins were captured with 791136 and eluted with increasing concentrations of 464917, 465178, 464392, and 465131. Increased total protein binding is observed for compound 464917 compared to 465178, 464932, and 465131.

### Example 30 Total Protein Binding, Activity and Toxicity with MOP Linkages

Modified oligonucleotides were evaluated for their total protein binding in cells. Cellular proteins were captured with the biotin-conjugated modified oligonucleotide 592590, which has a 5′-biotin-TEG, the sugar motif kkk-d(10)-kkk and the sequence GCTAGCCTCTGGATTT (SEQ ID NO: 115) and eluted with the modified oligonucleotides described in the table below. Eluted proteins were run on an SDS-PAGE gel and visualized. Decreased protein binding is observed for compounds with decreased toxicity compared to 558807, in particular for compounds 766654, 766655, and 766666.

TABLE 76
Modified oligonucleotides containing Two MOP linkages

| | Linkage Mod | | | SEQ |
|---|---|---|---|---|
| Compound | position in | | | ID |
| ID | central region | Target | Chemistry Notation | NO |
| 766653 | 1, 2 | CXCL12 | Gₖₛ^{m}CₖₛAₖₛT_{dx}G_{dx}T_{ds}T_{ds}^{m}C_{ds}T_{ds}^{m}C_{ds}A_{ds}^{m}C_{ds}A_{ds}TₖₛTₖₛAₖ | 18 |
| 766654 | 2, 3 | CXCL12 | Gₖₛ^{m}CₖₛAₖₛT_{ds}G_{dx}T_{dx}T_{ds}^{m}C_{ds}T_{ds}^{m}C_{ds}A_{ds}^{m}C_{ds}A_{ds}TₖₛTₖₛAₖ | 18 |
| 766655 | 3, 4 | CXCL12 | Gₖₛ^{m}CₖₛAₖₛT_{ds}G_{ds}T_{dx}T_{dx}^{m}C_{ds}T_{ds}^{m}C_{ds}A_{ds}^{m}C_{ds}A_{ds}TₖₛTₖₛAₖ | 18 |
| 766656 | 4, 5 | CXCL12 | Gₖₛ^{m}CₖₛAₖₛT_{ds}G_{ds}T_{ds}T_{dx}^{m}C_{dx}T_{ds}^{m}C_{ds}A_{ds}^{m}C_{ds}A_{ds}TₖₛTₖₛAₖ | 18 |
| 766657 | 5, 6 | CXCL12 | Gₖₛ^{m}CₖₛAₖₛT_{ds}G_{ds}T_{ds}T_{ds}^{m}C_{dx}T_{dx}^{m}C_{ds}A_{ds}^{m}C_{ds}A_{ds}TₖₛTₖₛAₖ | 18 |
| 766658 | 6, 7 | CXCL12 | Gₖₛ^{m}CₖₛAₖₛT_{ds}G_{ds}T_{ds}T_{ds}^{m}C_{ds}T_{dx}^{m}C_{dx}A_{ds}^{m}C_{ds}A_{ds}TₖₛTₖₛAₖ | 18 |
| 766659 | 7, 8 | CXCL12 | Gₖₛ^{m}CₖₛAₖₛT_{ds}G_{ds}T_{ds}T_{ds}^{m}C_{ds}T_{ds}^{m}C_{dx}A_{dx}^{m}C_{ds}A_{ds}TₖₛTₖₛAₖ | 18 |
| 766665 | 8, 9 | CXCL12 | Gₖₛ^{m}CₖₛAₖₛT_{ds}G_{ds}T_{ds}T_{ds}^{m}C_{ds}T_{ds}^{m}C_{ds}A_{dx}^{m}C_{dx}A_{ds}TₖₛTₖₛAₖ | 18 |
| 766664 | 9, 10 | CXCL12 | Gₖₛ^{m}CₖₛAₖₛT_{ds}G_{ds}T_{ds}T_{ds}^{m}C_{ds}T_{ds}^{m}C_{ds}A_{ds}^{m}C_{dx}A_{dx}TₖₛTₖₛAₖ | 18 |

A subscript “d” indicates an unmodified, 2′-β-D-deoxyribosyl sugar moiety. A subscript “k” indicates a cEt. A subscript “s” indicates a phosphorothioate internucleoside linkage. A superscript “m” before a C indicates 5-methyl Cytosine. A subscript “x” indicates a MOP internucleoside linkage.

In vitro activity and in vivo activity and toxicity experiments were performed essentially as described in Example 1. For in vivo toxicity studies, a single BALB/C mouse per dose of modified oligonucleotide was administered 16.7 mg/kg, 50 mg/kg, or 150 mg/kg modified oligonucleotide by subcutaneous injection and sacrificed 72 hours later. ALT levels were measured using an automated clinical chemistry analyzer. For the in vivo activity study in the table below, 1 BALB/C mouse per group was administered 1.8 mg/kg, 5.5 mg/kg, 16.7 mg/kg, 50 mg/kg or 150 mg/kg modified oligonucleotide subcutaneously and sacrificed after 24 hours.

TABLE 77
in vivo Activity and Toxicity

| | | in vivo | | |
|---|---|---|---|---|
| | MOP | CXCL12 | ALT @ | ALT @ |
| Compound | linkage | ED50 | 50 mg/kg | 150 mg/kg |
| ID | positions | (mg/kg) | (IU/L) | (IU/L) |
| PBS | n/a | n/a | 26 (@0 mg/kg) | |
| 558807 | n/a | 2.9 | 19,806 | death |
| 766653 | 1, 2 | 23.6 | 32 | 33 |
| 766654 | 2, 3 | 31.6 | 28 | 30 |
| 766655 | 3, 4 | 32.7 | 28 | 27 |
| 766656 | 4, 5 | 26.7 | 25 | 29 |
| 766657 | 5, 6 | 7.0 | 213 | 5,503 |
| 766658 | 6, 7 | 6.2 | 64 | 1,380 |
| 766659 | 7, 8 | 10.6 | 51 | 3,423 |
| 766665 | 8, 9 | 5.5 | 3,437 | 11,954 |
| 766664 | 9, 10 | 6.2 | 4,045 | death |

### Example 31 Self-Structure of Modified Oligonucleotides Complementary to CXCL12

Tm was determined for self-structures of modified oligonucleotides described in the examples above. Compounds in the table below are complementary to CXCL12 and have sequences corresponding to SEQ ID NO: 18-21. Tm was also determined for duplexes of the modified oligonucleotides described in the examples above in complex with a RNA 20-mer with the sequence GAUAAUGUGAGAACAUGCCU (SEQ ID NO: 116).

TABLE 78
Tm of Modified Oligonucleotides complementary
to CXCL12, Self-Structure and Duplex

| | linkage- | Sugar- | | |
|---|---|---|---|---|
| | altered | modification | | |
| | nucleotide | of altered | Tm | |
| | (position | nucleotide | (° C.) | Tm |
| Compound | in central | (position in | Self | (° C.) |
| ID | region) | central region) | structure | duplex |
| 558807 | none | none | 48.6 | 65.1 |
| 1061955 | none | inosine (2) | 32.9 | 57.5 |
| 766676 | MOP(1) | none | 44.6 | 63.3 |
| 766677 | MOP(2) | none | 45.3 | 63.5 |
| 766678 | MOP(3) | none | 47.9 | 63.1 |
| 766679 | MOP(4) | none | 47.1 | 62.6 |
| 766680 | MOP(5) | none | n.d. | 63 |
| 766681 | MOP(6) | none | n.d. | 62.9 |
| 766682 | MOP(7) | none | n.d. | 63.8 |
| 766683 | MOP(8) | none | n.d. | 63.3 |
| 766684 | MOP(9) | none | n.d. | 64.1 |
| 766685 | MOP(10) | none | n.d. | 63.9 |
| 936053 | none | 2′-OMe (2) | 49.0 | 67.0 |
| 828911 | none | 2′-MOE (2) | 48.2 | 66.8 |
| 1070041 | none | cEt (2) | 52.7 | 69.5 |
| 936051 | none | 2′-FANA (2) | 46.1 | 64.8 |
| 936052 | none | 2′-ribo-F (2) | 47.2 | 66.0 |
| 1123320 | none | 5′-(R)—Me (2) | 49.4 | 65.5 |
| 1123322 | none | 5′-(S)—Me (2) | 43.0 | 62.0 |
| 942943 | none | 5′-(R)—Me (3) | 47.3 | 62.3 |
| 957908 | none | 5′-(S)—Me (3) | 45.1 | 65.1 |
| 942944 | none | 5′-(R)—Me (4) | 49.5 | 62.3 |
| 957909 | none | 5′-(S)—Me (4) | 46.2 | 66.3 |
| 957910 | none | 5′-(R)-allyl (3) | 44.4 | 62.1 |
| 957911 | none | 5′-(R)-allyl (4) | 47.3 | 62.4 |
| 957912 | none | 5′-(S)-allyl (3) | 41.7 | 64.0 |
| 957913 | none | 5′-(S)-allyl (4) | 47.1 | 64.6 |
| 1069852 | none | pseudoU (2) | 24.4 | 54.4 |
| 1061328 | none | pseudoU (3) | 44.6 | 55.3 |
| 1215458 | none | β-L-DNA (2) | n.d. | 58 |
| 1215459 | none | β-L-DNA (3) | 43 | 59 |
| 1215460 | none | β-L-DNA (4) | 45 | 62 |
| 1215461 | none | α-L-DNA (3) | 41 | 63 |
| 1215462 | none | α-L-DNA (4) | 49 | 65 |

TABLE 79
Tm of Modified Oligonucleotide Self-Structure

| Compound ID | Target | Tm (° C.) |
|---|---|---|
| 449093 | SRB1 | <40 |
| 464917 | FXI | <40 |
| 482050 | PTEN | 33.4 |
| 508031 | SOD-1 | 58.9 |

### Example 32 2′-Modifications in 5′ and 3′-Regions of Modified Oligonucleotides

Modified oligonucleotides containing various sugar modification motifs were synthesized as indicated in the table below. The compounds in the table below are 100% complementary to mouse CXCL12, GENBANK NT_039353.7 truncated from 69430515 to 69445350 (SEQ ID NO: 1) at positions 6877 to 6892 (16-mers) or 6875 to 6894 (20-mers).

TABLE 80
modified oligonucleotides with 2′-sugar modifications

| | 2′- | 2′- | | |
|---|---|---|---|---|
| | modified | modified | | SEQ |
| Compound | sugars on | sugars on | | ID |
| ID | 5′-end | 3′-end | Chemistry Notation | NO |
| 558807 | kkk | kkk | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 1035522 | kkk | eee | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}T_{eS}TₑₛAₑ | 18 |
| 1035523 | eee | kkk | Gₑₛ^{m}CₑₛAₑₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 985648 | eee | eee | Gₑₛ^{m}CₑₛAₑₛT_{ds}G_{ds}T_{ds}T_{ds}^{m}C_{ds}T_{ds}^{m}C_{ds}A_{ds}^{m}C_{ds}A_{ds}TₑₛTₑₛAₑ | 18 |
| 1069842 | kkeee | eeekk | AₖₛGₖₛGₑₛ^{m}CₑₛAₑₛT_{ds}G_{ds}T_{ds}T_{ds}^{m}C_{ds}T_{ds}^{m}C_{ds}A_{ds}^{m}C_{ds}A_{ds}TₑₛTₑₛAₑₛTₖₛ^{m}Cₖ | 117 |
| 1069843 | kkeee | kkkkk | AₖₛGₖₛGₑₛ^{m}CₑₛAₑₛT_{ds}G_{ds}T_{ds}T_{ds}^{m}C_{ds}T_{ds}^{m}C_{ds}A_{ds}^{m}C_{ds}A_{ds}TₖₛTₖₛAₖₛTₖₛ^{m}Cₖ | 117 |
| 1069844 | kkkkk | eeekk | AₖₛGₖₛGₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₑₛTₑₛAₑₛTₖₛ^{m}Cₖ | 117 |
| 386864 | eeeee | eeeee | AₑₛGₑₛGₑₛ^{m}CₑₛAₑₛT_{ds}G_{ds}T_{ds}T_{ds}^{m}C_{ds}T_{ds}^{m}C_{ds}A_{ds}^{m}C_{ds}A_{ds}TₑₛTₑₛAₑₛTₑₛ^{m}Cₑ | 117 |
| 1069845 | kkkkk | kkkkk | AₖₛGₖₛGₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖₛTₖₛ^{m}Cₖ | 117 |
| 1069846 | eekkkk | kkkkk | A_{eS}GₑₛGₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖₛTₖₛ^{m}Cₖ | 117 |
| 1069847 | kkkkk | kkkee | A_{kS}GₖₛGₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}T_{kS}T_{kS}A_{kS}Tₑₛ^{m}Cₑ | 117 |

A subscript “d” indicates an unmodified, 2′-β-D-deoxyribosyl sugar moiety. A subscript “k” indicates a cEt. A subscript “s” indicates a phosphorothioate internucleoside linkage. A superscript “m” before a C indicates 5-methyl Cytosine. A subscript “e” indicates a 2′-MOE-β-D-ribofuranosyl sugar moiety. A subscript “m” indicates a 2′-O-methyl-β-D-ribofuranosyl sugar moiety.
For the in vitro study reported in the tables below, 3T3-L1 cells were electroporated with 27 nM, 80 nM, 250 nM, 740 nM, 2, 222 nM, 6,667 nM, or 20,000 nM of modified oligonucleotide and levels of CXCL12 were measured by RT-qPCR as described in Example 1 above. Caspase activation was measured as described in Example 4 above. Results are presented relative to the caspase activation in control cells not treated with modified oligonucleotide. Mislocalization of p54nrb was analyzed as described in Example 15 above. For experiments reported in the tables below, selected images were qualitatively analyzed and assigned a score of (−), indicating that no p54nrb mislocalization to the nucleolus was observed, or a score of (+) indicating that mislocalization of p54nrb to the nucleolus was observed in some cells, or (++) indicating that p54nrb mislocalizization to the nucleolus was observed in most or all cells. Treatment of HeLa cells with certain modified oligonucleotides caused a filamentous appearance of p54nrb in cells. This is indicated by a “f” in the table below.

TABLE 81
in vitro Activity and Toxicity

| Compound | Caspase | CXCL12 IC₅₀ | p54nrb |
|---|---|---|---|
| ID | (% control) | (nM) | mislocalization |
| 558807 | 1135 | 30 | ++ |
| 1035522 | 1261 | 35 | +, f |
| 1035523 | 244 | 100 | +, f |
| 985648 | 207 | 200 | −, f |
| 1069842 | 353 | 350 | +, f |
| 1069843 | 670 | 100 | ++ |
| 1069844 | 748 | 350 | + |
| 386864 | 1104 | 200 | −, f |
| 1069845 | 213 | 350 | ++ |
| 1069846 | 963 | 100 | + |
| 1069847 | 923 | 250 | + |

### Example 33 Effect of Treatment of b.END Cells with Modified Oligonucleotides

For the in vitro study reported in the tables below, b.END.3 cells were electroporated with 3.125, 6.25, 12.5, 25, or 50 nM of modified oligonucleotide 464917 (heptatotoxic) or 549148 (nontoxic). 549148 is a 3-10-3 cEt modified oligonucleotide with the sequence GGCTACTACGCCGTCA (SEQ ID NO: 118), which is not complementary to any known mouse gene. Expression levels of p21 and Gadd45a mRNA were measured after 0, 1, 2, 4, and 6 hours by RT-qPCR as described in Example 1.

TABLE 82
Relative mp21 mRNA dose response/time course in b.END cells

| | Dose | % Control mp21 mRNA | | | |
|---|---|---|---|---|---|
| Compound | (nM) | 1 hrs | 2 hrs | 4 hrs | 6 hrs |
| 464917 | 0 | 100 | 100 | 100 | 100 |
| 464917 | 3.125 | 102 | 134 | 147 | 174 |
| 464917 | 6.25 | 113 | 149 | 169 | 242 |
| 464917 | 12.50 | 107 | 141 | 199 | 250 |
| 464917 | 25.0 | 122 | 183 | 330 | 394 |
| 464917 | 50.0 | 113 | 210 | 399 | 427 |
| 549148 | 0 | 100 | 100 | 100 | 100 |
| 549148 | 3.125 | 111 | 42 | 140 | 107 |
| 549148 | 6.25 | 88 | 90 | 128 | 126 |
| 549148 | 12.50 | 120 | 86 | 119 | 109 |
| 549148 | 25.0 | 114 | 111 | 147 | 107 |
| 549148 | 50.0 | 111 | 94 | 126 | 119 |

TABLE 82a
Relative mGadd45a mRNA dose response/time
course in b.END cells

| | Dose | % Control mGadd45a mRNA | | | |
|---|---|---|---|---|---|
| Compound | (nM) | 1 hrs | 2 hrs | 4 hrs | 6 hrs |
| 464917 | 0 | 100 | 100 | 100 | 100 |
| 464917 | 3.125 | 87 | 88 | 203 | 396 |
| 464917 | 6.25 | 81 | 154 | 259 | 565 |
| 464917 | 12.50 | 85 | 173 | 331 | 905 |
| 464917 | 25.0 | 102 | 247 | 715 | 1586 |
| 464917 | 50.0 | 132 | 420 | 1376 | 3339 |
| 549148 | 0 | 100 | 100 | 100 | 100 |
| 549148 | 3.125 | 85 | 31 | 106 | 109 |
| 549148 | 6.25 | 72 | 95 | 103 | 125 |
| 549148 | 12.50 | 85 | 87 | 106 | 127 |
| 549148 | 25.0 | 85 | 103 | 144 | 123 |
| 549148 | 50.0 | 97 | 107 | 131 | 198 |

### Example 34 Nucleolar Delocalization of p54Nrb in Various Cell Lines

Cells were plated at 20,000 cells/well and transfected with Lipofectamine 2,000 and 60 nM of modified oligonucleotide 791143, compound 464917 labeled on the 3′-end with Cy3. Cells were visualized 6 hours after transfection.

TABLE 83
p54nrb nucleolar mislocalization

| Compound ID | Cell line | p54nrb nucleolar mislocalization |
|---|---|---|
| 791143 | U2OS | + |
| 791143 | NIH3T3 | ++ |

Cells were plated at 20,000 cells/well and transfected by free uptake with modified oligonucleotide 791143 as indicated in the table below. Cells were visualized 5 hours after transfection.

TABLE 84
p54nrb nucleolar mislocalization

| Compound ID | Cell line | p54nrb nucleolar mislocalization |
|---|---|---|
| 791143 | MHT | + |
| 791143 | HeLa | ++ |

Cells were plated at 20,000 cells/well and transfected by NEON electroporation at 1400V, 20 ms, 2 pulses with 60 nM modified oligonucleotide 813223, compound 464917 labeled on the 5′-end with Cy3. Cells were visualized 5 hours after transfection.

TABLE 85
p54nrb nucleolar mislocalization

| Compound ID | Cell line | p54nrb nucleolar mislocalization |
|---|---|---|
| 813223 | HeLa | ++ |

Cells were transfected with 60 nM modified oligonucleotide 813223 or compound 813225. Compound 813225 is the control oligonucleotide 549148 described above labeled on the 5′-end with Cy3. Cells were visualized 4 hours after transfection.

TABLE 85b
p54nrb nucleolar mislocalization

| Compound ID | Cell line | p54nrb nucleolar mislocalization |
|---|---|---|
| 813223 | b.END3 | ++ |
| 813225 | b.END3 | − |
| 813223 | NIH3T3 | + |
| 813223 | primary neuron | + |
| 813223 | primary hepatocyte | ++ |

### Example 35 Effect of Depletion of RNaseH1 on Toxicity of Modified Oligonucleotides

HeLa cells were plated at 150,000 cells/well and transfected with control siRNA targeting luciferase or siRNA targeted to RNaseH1 (s48357 or s48358 from ThermoFisher) at a final concentration of 3 nM using Lipofectamine RNAiMAX for 48 hours. Modified oligonucleotides 464917 and 549148 were added to the cells by free uptake. 549148 is a 3-10-3 cEt modified oligonucleotide with the sequence GGCTACTACGCCGTCA (SEQ ID NO: 118), which is not complementary to any known mouse gene. Confocal microscopy was used to visualize p54nrb localization as described in Example 15 above.

TABLE 86
in vitro p54nrb mislocalization

| | | p54nrb |
|---|---|---|
| Compound | | nucleolar |
| ID | siRNA | mislocalization |
| 464917 | mock | ++ |
| 464917 | RNaseH1a | − |
| 464917 | RNaseH1b | − |

TABLE 87
in vitro P21 Expression in HeLa cells

| Compound | | 0 nM | 3.125 nM | 6.25 nM | 12.5 nM | 25 nM | 50 nM |
|---|---|---|---|---|---|---|---|
| ID | siRNA | Expression level of P21 mRNA (% Control) | | | | | |
| 464917 | Luci | 90 | 134 | 141 | 143 | 171 | 201 |
| 464917 | H1 | 78 | 79 | 82 | 83 | 84 | 95 |
| 549148 | Luci | 100 | 119 | 105 | 84 | 94 | 82 |
| 549148 | H1 | 99 | 86 | 92 | 81 | 79 | 85 |

TABLE 88
in vitro Gadd45a Expression in HeLa cells

| Compound | | 0 nM | 3.125 nM | 6.25 nM | 12.5 nM | 25 nM | 50 nM |
|---|---|---|---|---|---|---|---|
| ID | siRNA | Expression level of P21 mRNA (% Control) | | | | | |
| 464917 | Luci | 73 | 119 | 126 | 179 | 270 | 463 |
| 464917 | H1 | 65 | 82 | 110 | 117 | 154 | 260 |
| 549148 | Luci | 100 | 89 | 107 | 102 | 97 | 83 |
| 549148 | H1 | 72 | 83 | 103 | 103 | 105 | 96 |

### Example 36 Protein Binding of Modified Oligonucleotides

Modified oligonucleotides described in the examples above were evaluated for their protein binding in cells. Cellular proteins were captured with the biotin-conjugated modified oligonucleotide 791136, which has a 5′-biotin-TEG, the sugar motif kkk-d(10)-kkk, a full phosphorothioate backbone, and the same sequence as 464917, GTCTGTGCATCTCTCC (SEQ ID NO: 22). Proteins were eluted with increasing concentrations of 464917 or 549148. Eluted proteins were run on an SDS-PAGE gel and analyzed by western blot for p54nrb, FUS, RNaseH1, SSBP1, Ku70, PSPC1, SND1, FUBP, NCL1, and Ku80. Band intensities are represented in the table below: −, no band; +, faint band; ++, medium band; +++, intense band.

TABLE 89
Protein binding of modified oligonucleotides

| | Relative band intensity | | Relative band intensity | |
|---|---|---|---|---|
| | 464917 | | 549148 | |
| Protein | 0.625 μM | 5 μM | 0.625 μM | 5 μM |
| p54nrb | − | +++ | − | + |
| FUS | − | ++ | − | − |
| RNaseH1 | − | ++ | − | + |
| SSBP1 | ++ | ++ | + | + |
| Ku70 | + | ++ | + | + |
| PSPC1 | − | + | − | + |
| SND1 | ++ | +++ | + | ++ |
| FUBP | + | ++ | + | + |
| NCL1 | + | ++ | + | + |
| Ku80 | + | ++ | ++ | ++ |

Total protein binding to 464917 and 549418 was tested using biotin-464917 or biotin-549148 to capture cellular proteins, which were then eluted with increasing concentrations of 464917 or 549148. The hepatotoxic compound 464917 shows increased global protein binding compared to 549148.

### Example 37 In Vitro Activity and Toxicity of Modified Oligonucleotides Comprising Modified Internucleoside Linkages

Modified oligonucleotides were designed based on the control oligonucleotide 558807, described in Example 1 herein and synthesized using standard procedures. Modified internucleoside linkages (1 or 2) were positioned at various positions within the central region of the oligonucleotides as illustrated below. The resulting modified oligonucleotides were tested for their ability to inhibit CXCL12 (Chemokine ligand 12) and Raptor expression levels. The potency of the modified oligonucleotides was evaluated and compared to the control oligonucleotide.

The modified oligonucleotides were tested in vitro in mouse b.END cells by electroporation. Cells at a density of 20,000 cells per well are transfected using electroporation with 0.027, 0.082, 0.25, 0.74, 2.22, 6.67 and 20 uM concentrations of each of the oligonucleotides listed below. After a treatment period of approximately 24 hours, RNA is isolated from the cells and mRNA levels are measured by quantitative real-time PCR and the CXCL12 mRNA and Raptor mRNA levels are adjusted according to total RNA content, as measured by RIBOGREEN®.

TABLE 90
Modified Oligonucleotides

| | Linkage-altered | | | |
|---|---|---|---|---|
| Com- | nucleotide | | | SEQ |
| pound | position in | | | ID |
| ID | central region | Linkage mod “x” | Chemistry Notation | NO |
| 558807 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 857528 | 3 | THP phosphotriester | Gₖₛ^{m}CₖₛAₖₛT_{ds}G_{ds}TₓT_{ds}^{m}C_{ds}T_{ds}^{m}C_{ds}A_{ds}^{m}C_{ds}A_{ds}TₖₛTₖₛAₖ | 18 |
| 857529 | 3 | isopropyl- | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ | 18 |
| | | phosphotriester | | |
| 857530 | 3 | isobutylphosphonate | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ | 18 |
| 857505 | 3 | isopropylphosphonate | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ | 18 |
| 883401 | 3 | amide-3 | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ | 18 |
| 883521 | 3 | formacetal | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ | 18 |
| 857532 | 4 | THP phosphotriester | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ | 18 |
| 857533 | 4 | isopropyl- | Gₖₛ^{m}CₖₛAₖₛT_{ds}G_{ds}T_{ds}T_{dx}^{m}C_{ds}T_{ds}^{m}C_{ds}A_{ds}^{m}C_{ds}A_{ds}TₖₛTₖₛAₖ | 18 |
| | | phosphotriester | | |
| 857531 | 4 | isopropylphosphonate | Gₖₛ^{m}CₖₛAₖₛT_{ds}G_{ds}T_{ds}T_{dx}^{m}C_{ds}T_{ds}^{m}C_{ds}A_{ds}^{m}C_{ds}A_{ds}TₖₛTₖₛAₖ | 18 |
| 857534 | 4 | isobutylphosphonate | Gₖₛ^{m}CₖₛAₖₛT_{ds}G_{ds}T_{ds}T_{dx}^{m}C_{ds}T_{ds}^{m}C_{ds}A_{ds}^{m}C_{ds}A_{ds}TₖₛTₖₛAₖ | 18 |
| 857537 | 3,4 | isopropylphosphonate | Gₖₛ^{m}CₖₛAₖₛT_{ds}G_{ds}T_{dx}T_{dx}^{m}C_{ds}T_{ds}^{m}C_{ds}A_{ds}^{m}C_{ds}A_{ds}TₖₛTₖₛAₖ | 18 |
| 857540 | 3,4 | isobutylphosphonate | Gₖₛ^{m}CₖₛAₖₛT_{ds}G_{ds}T_{dx}T_{dx}^{m}C_{ds}T_{ds}^{m}C_{ds}A_{ds}^{m}C_{ds}A_{ds}TₖₛTₖₛAₖ | 18 |
| 857538 | 3,4 | THP phosphotriester | Gₖₛ^{m}CₖₛAₖₛT_{ds}G_{ds}T_{dx}T_{dx}^{m}C_{ds}T_{ds}^{m}C_{ds}A_{ds}^{m}C_{ds}A_{ds}TₖₛTₖₛAₖ | 18 |

A subscript “d” indicates a an unmodified, 2′-β-D-deoxyribosyl sugar moiety. A subscript “k” indicates a cEt. A subscript “s” indicates a phosphorothioate internucleoside linkage. A superscript “m” before a C indicates 5-methyl Cytosine. A subscript “x” indicates a modified internucleoside linkage as indicated in the “linkage mod x” column. These linkages are illustrated below.

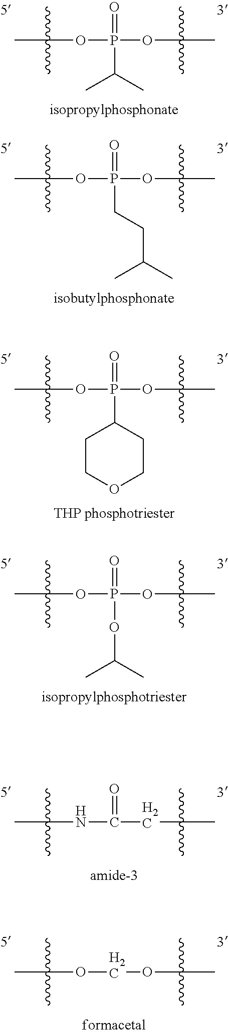

The half maximal inhibitory concentration (IC₅₀) of each oligonucleotide listed above was calculated by plotting the concentration of oligonucleotide versus the percent inhibition of CXCL12 mRNA or Raptor mRNA expression achieved at each concentration, and noting the concentration of oligonucleotide at which 50% inhibition of CXCL12 mRNA expression is achieved compared to the control. The results are presented in the table below.

TABLE 91
In vitro Activity and Toxicity

| Compound | IC₅₀ (μM) | Raptor % | Raptor |
|---|---|---|---|
| ID | CXCL12 | Control (4 μM) | IC50* (μM) |
| 558807 | 0.17 | 47 | 3.7 |
| 857505 | 0.15 | 82 | >4 |
| 857530 | 0.32 | 87 | >4 |
| 857528 | 0.23 | 110 | >4 |
| 857529 | 1.09 | 74 | >4 |
| 883401 | 30 | 65 | >4 |
| 883521 | 0.40 | 94 | >4 |
| 857531 | 0.27 | 99 | >4 |
| 857534 | 0.12 | 57 | >4 |
| 857532 | 0.16 | 69 | >4 |
| 857533 | 0.10 | 61 | >4 |
| 857537 | 1.4 | 82 | >4 |
| 857540 | 0.48 | 65 | >4 |
| 857538 | 0.33 | 110 | >4 |
| 857539 | 0.13 | 74 | >4 |

*IC50 values can only be calculated when less than the highest dose in the experiment, in this case, 4 μM

### Example 38

Modified oligonucleotides were designed based on 558807. Each modified oligonucleotide has a modified internucleoside linkage positioned between nucleosides 3 and 4 counting from the 5′-gap unction (not including the 3 cEt modified nucleosides in the 5′-wing) as illustrated below. Each of the modified oligonucleotides is conjugated with a HPPO-GalNAc conjugate group at the 3′-end as illustrated below. The oligonucleotides were evaluated for reduction in CXCL12 (Chemokine ligand 12) mRNA expression levels in vivo. The transaminase levels (ALT and AST) for each dose were also measured.

Six week old BALB/C mice (purchased from Charles River) were injected subcutaneously once at dosage 0.2, 0.6, 1.8 or 50 mg/kg with the modified oligonucleotides shown below or with saline control. For compound 855156, mice were injected with 0.21, 0.62, 1.85,or 5.56 mg/kg modified oligonucleotide. Each treatment group consisted of 3 animals. The mice were sacrificed 72 hours following administration, and organs and plasma were harvested for further analysis.

TABLE 92
Modified Oligonucleotides

| | Linkage-altered | | | |
|---|---|---|---|---|
| | nucleotide | | | SEQ |
| Compound | position in | | | ID |
| ID | central region | Linkage mod “x” | Chemistry Notation | NO |
| 895566 | 3 | isopropyl- | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | phosphonate | HPPO -GalNAc | |
| 895567 | 3 | THP | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | phosphotriester | HPPO -GalNAc | |
| 895568 | 3 | isopropyl- | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | phosphotriester | HPPO -GalNAc | |
| 895569 | 3 | isobutyl- | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | phosphonate | HPPO -GalNAc | |
| 895570 | 3 | formacetal | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |
| 913196 | 3 | amide-3 | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |
| 920046 | 3 | TANA | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |
| 951972 | 3 | (R)-MOP | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |
| 951973 | 3 | (S)-MOP | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |
| 974343 | 3 | alt- | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | thioformacetal | HPPO -GalNAc | |
| 974344 | 3 | glycine amide | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |
| 1011295 | 3 | thioformacetal | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |
| 1011296 | 3 | MMI | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖ- | 18 |
| | | | HPPO -GalNAc | |

A subscript “d” indicates an unmodified, 2′-β-D-deoxyribosyl sugar moiety. A subscript “k” indicates a cEt. A subscript “s” indicates a phosphorothioate internucleoside linkage. A superscript “m” before a C indicates 5-methyl Cytosine. A subscript “x” indicates a modified internucleoside linkage as indicated in the “linkage mod x” column. These linkages are illustrated above and below.

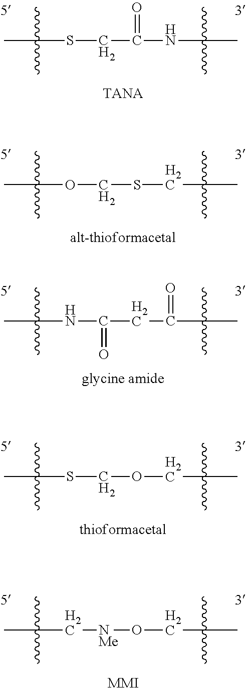

Each modified oligonucleotide in the study includes a 3′-HPPO-GalNAc conjugate group which is attached to the 3′-oxygen of the oligomeric compound. The 3′-HPPO-GalNAc conjugate group is illustrated below wherein the phosphate group is attached to the 3′-oxygen atom:

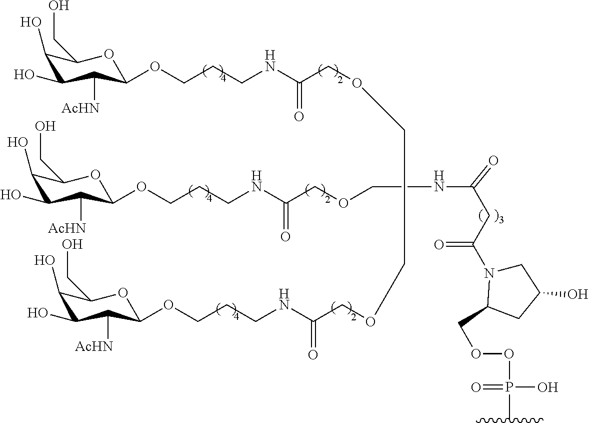

Liver tissues were homogenized and mRNA levels were quantitated using real-time PCR and normalized to RIBOGREEN as described herein. Plasma chemistry markers such as liver transaminase levels, alanine aminotranferase (ALT) in serum were measured relative to saline injected mice.

The ED₅₀ values were calculated by plotting the concentrations of oligonucleotides used versus the percent inhibition of CXCL12 mRNA expression achieved at each concentration, and noting the concentration of oligonucleotide at which 50% inhibition of CXCL12 mRNA expression was achieved compared to the control.

TABLE 93
In vivo Toxicity

| | Linkage-altered | | ALT (at |
|---|---|---|---|
| | nucleotideposition | | 50 |
| Compound ID | in Central region | Linkage Mod | mg/kg) |
| 855156* | n/a | n/a | 4298** |
| 855161 | 3 | MOP | 31 |
| 895566 | 3 | isopropylphosphonate | 24 |
| 895567 | 3 | THP phosphotriester | 25 |
| 895568 | 3 | isopropylphosphotriester | 38 |
| 895569 | 3 | isobutylphosphonate | 28 |
| 895570 | 3 | formacetal | 31 |
| 913196 | 3 | amide-3 | 29 |
| 920046 | 3 | TANA | 24 |
| 951972 | 3 | (R)-MOP | 47 |
| 951973 | 3 | (S)-MOP | 45 |
| 974343 | 3 | alt-thioacetal | 39 |
| 974344 | 3 | glycine amide | 30 |
| 1011295 | 3 | thioacetal | 38 |
| 1011296 | 3 | MMI | 56 |

*Described in Table 25 above
**Values determined in an independent experiment and shown for comparison; ALT value is at 5.56 mg/kg modified oligonucleotide

TABLE 93b
In Vivo CXCL12 mRNA levels

| | 0.21 | 0.62 | 1.85 | 5.56 | 50 |
|---|---|---|---|---|---|
| | mg/kg | mg/kg | mg/kg | mg/kg | mg/kg |
| Compound ID | Expression level of CXCL12 mRNA (% Control) | | | | |
| 855156* | 81 | 63 | 45 | 31 | n.d. |
| 895566 | 68 | 55 | 42 | n.d. | 22 |
| 895567 | 59 | 50 | 36 | n.d. | 18 |
| 895568 | 69 | 49 | 37 | n.d. | 17 |
| 895569 | 72 | 51 | 41 | n.d. | 18 |
| 895570 | 68 | 50 | 38 | n.d. | 17 |
| 913196 | 62 | 48 | 44 | n.d. | 19 |
| 920046 | 80 | 58 | 58 | n.d. | 25 |
| 855161 | 67 | 51 | 38 | 32 | 21 |
| 951972 | 77 | 61 | 39 | 29 | 20 |
| 951973 | 81 | 59 | 37 | 32 | 19 |
| 974343 | 86 | 56 | 37 | 27 | 16 |
| 974344 | 79 | 69 | 44 | 34 | 23 |
| 1011295 | 78 | 62 | 44 | 31 | 30 |
| 1011296 | 77 | 63 | 49 | 51 | 29 |

### Example 39 Synthesis of 5′-(R)-Ethyl and 5′-(S)-Ethyl Phosphoramidites

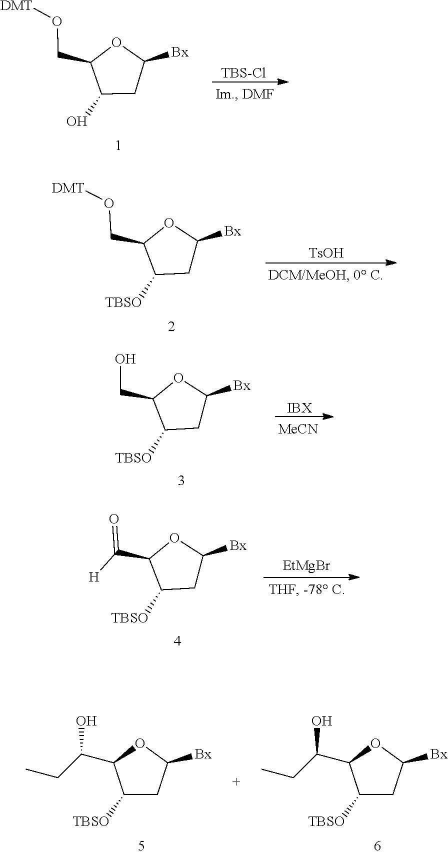

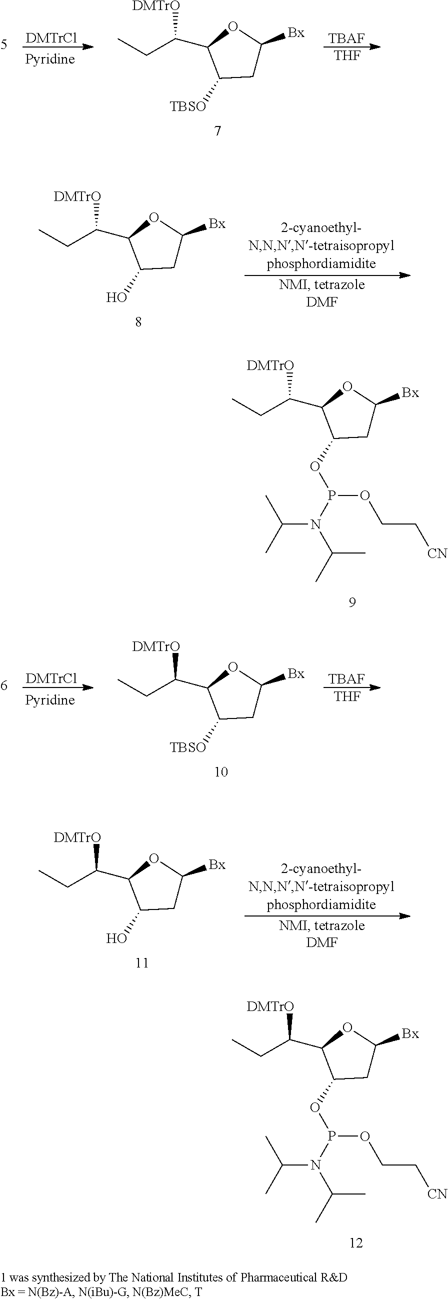

5′-(R)-ethyl and 5′-(S)-ethyl phosphordiamidtes were prepared as per the scheme illustrated above.

### Example 40 Time Course of Modified Oligonucleotides in HeLa Cells

HeLa cells were transfected with a modified oligonucleotide listed in the tables below. At 0, 0.5, 1, 2, 4, and 6 hours after transfection, cells were lysed and mRNA was isolated and analyzed by RT-qPCR. Primer probe set HTS3934 (forward sequence: TGGAGACTCTCAGGGTCGAAA, SEQ ID NO: 122; reverse sequence: GGCGTTTGGAGTGGTAGAAATC, SEQ ID NO: 123; probe sequence: CGGCGGCAGACCAGCATGAC, SEQ ID NO: 124) was used to detect human p21 mRNA, and primer probe set HS00169255_m1 (ThermoFisher)) was used to detect human Gadd45a mRNA. Results are normalized to untreated cells.

TABLE 94
Relative hp21 mRNA timecourse in HeLa cells

| | % Control human p21 mRNA | | | | | |
|---|---|---|---|---|---|---|
| Compound | 0 hrs | 0.5 hrs | 1 hrs | 2 hrs | 4 hrs | 6 hrs |
| 464917 | 100 | 123 | 133 | 241 | 259 | 557 |
| 558807 | 97.8 | 113 | 135 | 187 | 253 | 528 |
| 549148 | 120 | 120 | 129 | 187 | 138 | 147 |
| 549139 | 102 | 125 | 124 | 143 | 133 | 213 |

TABLE 95
Relative hGadd45a mRNA timecourse in HeLa cells

| | % Control human Gadd45a mRNA | | | | | |
|---|---|---|---|---|---|---|
| Compound | 0 hrs | 0.5 hrs | 1 hrs | 2 hrs | 4 hrs | 6 hrs |
| 464917 | 100 | 157 | 281 | 375 | 632 | 746 |
| 558807 | 105 | 188 | 227 | 297 | 261 | 412 |
| 549148 | 106 | 156 | 200 | 231 | 156 | 180 |
| 549139 | 94 | 157 | 213 | 229 | 167 | 237 |

HeLa cells were transfected with various concentrations of modified oligonucleotide as indicated in the table below. At 0, 1, 2, 4, 6, and 8 hours after transfection, cells were lysed and mRNA was isolated and analyzed by RT-qPCR as described above.

TABLE 96
Relative hp21 mRNA dose response/time course in HeLa cells

| | | % Control human p21 mRNA | | | | | |
|---|---|---|---|---|---|---|---|
| | Dose | 0 | 1 | 2 | 4 | 6 | 8 |
| Compound | (nM) | hrs | hrs | hrs | hrs | hrs | hrs |
| 464917 | 0 | 100 | 114 | 87 | 105 | 107 | 97 |
| 464917 | 3.125 | 100 | 109 | 76 | 111 | 179 | 126 |
| 464917 | 6.25 | 100 | 110 | 86 | 113 | 219 | 159 |
| 464917 | 12.50 | 100 | 112 | 86 | 126 | 287 | 239 |
| 464917 | 25.0 | 100 | 110 | 98 | 153 | 313 | 399 |
| 464917 | 50.0 | 100 | 96 | 94 | 165 | 392 | 490 |
| 464917 | 100.0 | 100 | 108 | 106 | 191 | 450 | 600 |
| 464917 | 200.0 | 100 | 99 | 100 | 230 | 510 | 660 |
| 549148 | 0 | 100 | 89 | 106 | 113 | 106 | 79 |
| 549148 | 3.125 | 100 | 105 | 100 | 117 | 126 | 96 |
| 549148 | 6.25 | 100 | 88 | 99 | 128 | 115 | 84 |
| 549148 | 12.50 | 100 | 95 | 108 | 107 | 115 | 107 |
| 549148 | 25.0 | 100 | 95 | 123 | 130 | 140 | 111 |
| 549148 | 50.0 | 100 | 101 | 111 | 122 | 131 | 114 |
| 549148 | 100.0 | 100 | 98 | 89 | 131 | 104 | 100 |
| 549148 | 200.0 | 100 | 93 | 95 | 163 | 102 | 99 |

TABLE 97
Relative hGadd45a mRNA dose response/time course in HeLa cells

| | | % Control human p21 mRNA | | | | | |
|---|---|---|---|---|---|---|---|
| | Dose | 0 | 1 | 2 | 4 | 6 | 8 |
| Compound | (nM) | hrs | hrs | hrs | hrs | hrs | hrs |
| 464917 | 0 | 100 | 97 | 80 | 109 | 110 | 88 |
| 464917 | 3.125 | 100 | 117 | 95 | 156 | 208 | 170 |
| 464917 | 6.25 | 100 | 121 | 111 | 176 | 176 | 205 |
| 464917 | 12.50 | 100 | 139 | 126 | 165 | 271 | 261 |
| 464917 | 25.0 | 100 | 147 | 136 | 176 | 279 | 420 |
| 464917 | 50.0 | 100 | 130 | 171 | 203 | 368 | 700 |
| 464917 | 100.0 | 100 | 143 | 194 | 261 | 835 | 1234 |
| 464917 | 200.0 | 100 | 113 | 198 | 213 | 890 | 1111 |
| 549148 | 0 | 100 | 98 | 104 | 104 | 111 | 99 |
| 549148 | 3.125 | 100 | 124 | 133 | 120 | 132 | 133 |
| 549148 | 6.25 | 100 | 151 | 140 | 155 | 160 | 142 |
| 549148 | 12.50 | 100 | 159 | 159 | 131 | 120 | 144 |
| 549148 | 25.0 | 100 | 173 | 172 | 148 | 156 | 180 |
| 549148 | 50.0 | 100 | 155 | 170 | 164 | 104 | 164 |
| 549148 | 100.0 | 100 | 140 | 129 | 141 | 160 | 190 |
| 549148 | 200.0 | 100 | 121 | 115 | 128 | 107 | 185 |

### Example 41 Time Course of Toxicity of Modified Oligonucleotide 464917 In Vivo

The modified oligonucleotide 464917 was administered subcutaneously at 11, 33, or 100 mg/kg to 9 BALB/C mice per dosing group. Three mice from each group were sacrificed at 24 hours, three at 48 hours, and the last three at 72 hours after dosing. mRNA was isolated and analyzed as described in Example 1. ALT values in plasma were obtained using a clinical chemistry analyzer.

TABLE 98
Time Course of Activity and Toxicity in mice

| | Dose | 24 hrs | 48 hrs | 72 hrs | 24 hrs | 48 hrs | 72 hrs |
|---|---|---|---|---|---|---|---|
| Compound | (mg/kg) | ALT | | | mFXI mRNA | | |
| 464917 | 0 | 44 | 58 | 29 | 100 | 100 | 100 |
| 464917 | 11 | 40 | 132 | 311* | 20 | 17 | 11* |
| 464917 | 33 | 98 | 2015 | 8072 | 2.7 | 2.6 | 5.7 |
| 464917 | 100 | 168 | 12261 | 26659* | 1.7 | 0.5 | 0.07** |

*Data represents a single mouse
**Data represents the average of two mice

TABLE 99
Time Course of Toxicity in mice

| | Dose | 24 hrs | 48 hrs | 72 hrs | 24 hrs | 48 hrs | 72 hrs |
|---|---|---|---|---|---|---|---|
| Compound | (mg/kg) | mP21 mRNA | | | mTnfrsf10b mR | | NA |
| 464917 | 0 | 100 | 100 | 100 | 100 | 100 | 100 |
| 464917 | 11 | 518 | 607 | 2060* | 272 | 845 | 3401* |
| 464917 | 33 | 6451 | 1846 | 5221 | 2071 | 5333 | 7013 |
| 464917 | 100 | 163667 | 4067 | 4910** | 5451 | 12293 | 7402** |

*Data represents a single mouse
**Data represents the average of two mice

### Example 42 Nucleolar Delocalization of p54Nrb In Vivo

Compound 959265 is compound 464917 conjugated to a Cy3 on the 5′-end and HPPO-GalNAc on the 3′-end. Balb/c mice were administered 15 mg/kg of 959265 by subcutaneous injection. Hepatocytes were isolated and purified 40 hours after administration of modified oligonucleotide and plated on a confocal dish for 6-7 hours. After 6-7 hours, cells were fixed with formaldehyde and stained for p54nrb with immunofluorescent staining for p54nrb. Levels of FXI and p21 were detected by RT-qPCR as described above.

A single dose of 959265 at 15 mg/kg reduced FXI to 10.6% of control values. Levels of p21 mRNA were upregulated 1,046%. Isolated hepatocytes were observed to contain p54nrb that had been localized to the nucleolus or no detectable p54nrb.

### Example 43 Nucleolar Delocalization of p54Nrb In Vivo

Balb/c mice were administered 100 mg/kg of 464917 or 549148 by subcutaneous injection. Hepatocytes were isolated and purified 16 hours after administration of modified oligonucleotide and plated on a confocal dish for 1-2 hours. After 1-2 hours, cells were fixed with formaldehyde and stained for p54nrb with immunofluorescent staining for p54nrb.

Localization of p54nrb to the nucleolus of hepatocytes was observed for compound 464917 but not for compound 549148.

### Example 44 In Vivo Activity and Toxicity of Compounds Containing a MOP Neutral Linkage

Six week old BALB/C mice (purchased from Charles River) were injected subcutaneously once at dosage 0.2, 0.6, 1.8 5.4,or 50 mg/kg with the modified oligonucleotides shown below or with saline control. For compound 855156, mice were injected with 0.2, 0.6, 1.8, 5.4, or 15 mg/kg modified oligonucleotide. Each treatment group consisted of 3 animals. The mice were sacrificed 72 hours following administration, and organs and plasma were harvested for further analysis.

Liver tissues were homogenized and mRNA levels were quantitated using real-time PCR and normalized to RIBOGREEN as described herein. Plasma chemistry markers such as liver transaminase levels, alanine aminotranferase (ALT) in serum were measured relative to saline injected mice.

The ED₅₀ values were calculated by plotting the concentrations of oligonucleotides used versus the percent inhibition of CXCL12 mRNA expression achieved at each concentration, and noting the concentration of oligonucleotide at which 50% inhibition of CXCL12 mRNA expression was achieved compared to the control.

TABLE 100
Modified Oligonucleotides

| | position of | | | |
|---|---|---|---|---|
| | linkage-altered | | | SEQ |
| Compound | nucleotide in | Linkage | | ID |
| ID | central region | mod | Chemistry Notation | NO |
| 869742 | n/a | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖₛTₖ- | 125 |
| | | | HPPO -GalNAc | |
| 898384 | 3 | MOP | Gₖₛ^{m}CₖₛAₖₛT_{ds}G_{ds}T_{dx}T_{ds}^{m}C_{ds}T_{ds}^{m}C_{ds}A_{ds}^{m}C_{ds}A_{ds}TₖₛTₖₛAₖₛTₖ- | 125 |
| | | | HPPO -GalNAc | |
| 898385 | 2, 3 | MOP | Gₖₛ^{m}CₖₛAₖₛT_{ds}G_{dx}T_{dx}T_{ds}^{m}C_{ds}T_{ds}^{m}C_{ds}A_{ds}^{m}C_{ds}A_{ds}TₖₛTₖₛAₖₛTₖ- | 125 |
| | | | HPPO -GalNAc | |

A subscript “d” indicates an unmodified, 2′-β-D-deoxyribosyl sugar moiety. A subscript “k” indicates a cEt. A subscript “s” indicates a phosphorothioate internucleoside linkage. A superscript “in” before a C indicates 5-methyl Cytosine. A subscript “x” indicates a MOP internucleoside linkage.

TABLE 101
In vivo Toxicity

| | Linkage Mod | | | |
|---|---|---|---|---|
| | position in | Linkage | ALT at | ALT at |
| Compound ID | Central region | Mod | 15 mg/kg | 50 mg/kg |
| 855156 | n/a | n/a | 9,639 | n/a |
| 869742 | n/a | n/a | 2,001 | n/a |
| 898384 | 3 | MOP | 30 | 32 |
| 898385 | 2, 3 | MOP | 32 | 30 |

TABLE 102
In Vivo CXCL12 mRNA levels

| | 0.2 | 0.6 | 1.8 | 5.4 | 15 | 50 |
|---|---|---|---|---|---|---|
| Compound | mg/kg | mg/kg | mg/kg | mg/kg | mg/kg | mg/kg |
| ID | Expression level of CXCL12 mRNA (% Control) | | | | | |
| 855156 | 64 | 42 | 23 | 19 | 16 | n/a |
| 869742 | 87 | 58 | 32 | 23 | 18 | n/a |
| 898384 | 87 | 91 | 49 | 40 | 36 | 31 |
| 898385 | 91 | 90 | 64 | 64 | 55 | 41 |

### Example 45

Modified oligonucleotides were tested for toxicity in vivo in Balb/c mice.

TABLE 103
Modified Oligonucleotides

| Com- | | SEQ |
|---|---|---|
| pound | | ID |
| ID | Chemistry notation | NO: |
| 575013 | ^{m}CₖₛAₖₛGₖₛA_{ds}T_{ds}A_{ds}T_{ds}A_{ds}G_{ds}G_{ds}A_{ds}^{m}C_{ds}T_{ds}GₖₛGₖₛAₖ | 110 |
| 465131 | TₖₛGₖₛ^{m}Cₖₛ^{m}C_{ds}A_{ds}^{m}C_{ds}^{m}C_{ds}G_{ds}T_{ds}A_{ds} | 114 |
| | G_{ds}A_{ds}^{m}C_{ds}Aₖₛ^{m}CₖₛGₖ | |
| 549139 | GₖₛAₖₛ^{m}CₖₛG_{ds}^{m}C_{ds}G_{ds}^{m}C_{ds}^{m}C_{ds}T_{ds}G_{ds}A_{ds}A_{ds}G_{ds}GₖₛTₖₛTₖ | 111 |
| 464932 | GₖₛGₖₛAₖₛ^{m}C_{ds}A_{ds}^{m}C_{ds}A_{ds}T_{ds}T_{ds}G_{ds}G_{ds}^{m}C_{ds}^{m} | 112 |
| | C_{ds}Aₖₛ^{m}CₖₛAₖ | |

A subscript “d” indicates an unmodified, 2′-β-D-deoxyribosyl sugar moiety. A subscript “k” indicates a cEt modified sugar moiety. A subscript “s” indicates a phosphorothioate internucleoside linkage. A superscript “m” before a C indicates 5-methyl Cytosine.

For in vivo toxicity studies, 3 BALB/c mice per group were administered the indicated dose of modified oligonucleotide by subcutaneous injection and sacrificed after 72 hours. Four animals were administered saline to serve as a control. RT-PCR was performed as described in Example 1 to determine mRNA levels of CXCL12, P21, Tnfrsf10b, and Gadd45a. Plasma levels of ALT were measured using an automated clinical chemistry analyzer. Increased ALT is indicative of acute liver toxicity.

For the in vitro toxicity study in the table below, the caspase assay was performed essentially as described in Example 8 in 3T3-L1 cells. The caspase assay was performed in HeLa cells by free uptake at 2 μM modified oligonucleotide and in b.END3 cells by free uptake at 50 μM modified oligonucleotide.

TABLE 104
In vivo and in vitro toxicity of modified oligonucleotides

| | | | | | in vitro | in vitro | in vitro |
|---|---|---|---|---|---|---|---|
| | | | | | caspase | caspase | caspase |
| | in vivo | | in vivo | in vivo | @20 μM, | @2 μM, | @50 μM, |
| Compound | Dose | ALT | P21 | Tnfrsf10b | 3T3-L1 | HeLa | b.END |
| ID | (mg/kg) | (IU/L) | (% control) | (% control) | (% control) | (% control) | (% control) |
| 575013 | 100 | 12 | 54 | 105 | 237 | 100 | 140 |
| 465131 | 100 | 21 | 111 | 164 | 114 | 119 | n.d. |
| 549139 | 100 | 24 | 118 | 135 | 125 | 111 | 113 |
| 549148 | 100 | 24 | 72 | 83 | 184 | 121 | 159 |
| 464932 | 100 | 5 | 150 | 180 | 280 | 113 | 187 |
| 449093 | 33 | 2324 | 42802 | 3835 | 2703 | 306 | 783 |
| | 100 | 9983 | 150994 | 3744 | | | |
| 482050 | 33 | 1470 | 7890 | 4725 | 1502 | 203 | 439 |
| | 100 | 6555 | 10430 | 4232 | | | |
| 508031 | 33 | 648 | 2980 | 2239 | 1082 | 255 | 357 |
| | 100 | 18550 | 8909 | 6678 | | | |
| 558807 | 17 | 1877 | 2763 | 1168 | 910 | 408 | 413 |
| | 51 | 9510 | 11904 | 6831 | | | |
| 464917 | 11 | 601 | 6098 | 3516 | 1724 | 219 | 552 |
| | 33 | 13920 | 9590 | 7731 | | | |

### Example 46 Time Course of Toxicity and Activity of Modified Oligonucleotide 464932 or 464917 In Vivo

The modified oligonucleotide 464932, described in Example 45 above, or 464917, described in Example 4 above, was administered subcutaneously at 33 mg/kg to BALB/C mice. Three mice from each dosing group were sacrificed at each indicated time point and mRNA was isolated and analyzed as described in Example 1. ALT values in plasma were obtained using a clinical chemistry analyzer and were normalized to saline-treated animals.

TABLE 105
Time Course of Activity and Toxicity in mice

| | | 8 | 12 | 16 | 24 | 48 | 72 |
|---|---|---|---|---|---|---|---|
| Treatment | Measurement | hrs | hrs | hrs | hrs | hrs | hrs |
| PBS | ALT | 20 | 41 | 40 | 40 | 44 | 41 |
| 464932 | ALT | 49 | 64 | 58 | 47 | 39 | 108 |
| | mFXI | 174 | 104 | 40 | 53 | 19 | 12 |
| | mP21 | 94 | 115 | 71 | 182 | 47 | 185 |
| | mTnfrsf10b | 133 | 101 | 112 | 108 | 117 | 140 |
| 464917 | ALT | 39 | 49 | 53 | 41 | 1903 | 13917 |
| | mFXI | 100 | 56 | 12 | 19 | 4 | 5 |
| | mP21 | 138 | 391 | 829 | 3751 | 1854 | 12716 |
| | mTnfrsf10b | 118 | 221 | 714 | 1250 | 6369 | 8781 |

### Example 47 Time Course of Toxicity and Activity of Modified Oligonucleotide 558807 or 558765 In Vivo

Modified oligonucleotide 558765 is a 3-10-3 cEt gapmer with a full phosphorothioate backbone and the sequence A^{m}CAT^{m}CTT^{m}CAGAT^{m}CATT (SEQ ID NO: 144). The modified oligonucleotide 558807 or 558765 was administered subcutaneously at 51 mg/kg to BALB/C mice. Three mice from each dosing group were sacrificed at each indicated time point and mRNA was isolated and analyzed as described in Example 1. ALT values in plasma were obtained using a clinical chemistry analyzer and were normalized to saline-treated animals.

TABLE 106
Time Course of Activity and Toxicity in mice

| | | 8 | 12 | 16 | 24 | 48 | 72 |
|---|---|---|---|---|---|---|---|
| Treatment | Measurement | hrs | hrs | hrs | hrs | hrs | hrs |
| PBS | ALT | 21 | 42 | 40 | 40 | 44 | 41 |
| 558765 | ALT | 34 | 53 | 46 | 92 | 33 | 36 |
| | mCXCL12 | 109 | 94 | 20 | 54 | 29 | 26 |
| | mP21 | 143 | 116 | 92 | 209 | 48 | 404 |
| | mTnfrsf10b | 106 | 70 | 118 | 98 | 154 | 215 |
| 558807 | ALT | 36 | 50 | 53 | 36 | 1888 | 7272 |
| | mCXCL12 | 43 | 18 | 5 | 10 | 3 | 3 |
| | mP21 | 136 | 142 | 86 | 580 | 1573 | 1642 |
| | mTnfrsf10b | 101 | 148 | 236 | 292 | 3375 | 7454 |

### Example 48 Toxicity Improvement In Vivo with Incorporation 2′-OMe Modified-Nucleoside in the Central Region

BALB/c mice were administered 1.8, 5.5, 16.7, or 50 mg/kg of 558807 or 1.8, 5.5, 16.7, 50, 100, 200, or 300 mg/kg of 936053 and sacrificed after 72 hours. Plasma levels of ALT were measured with a clinical chemistry analyzer and mRNA was isolated and analyzed as described in Example 1. Therapeutic index (TI) was calculated as the maximum non-toxic dose divided by the ED50. Compound 936053 differs from compound 558807 only in the presence of a 2′-OMe group at position 5 from the 5′ end of the compound, or position 2 of the central region.

TABLE 107
in vivo dose response

| | | | in vivo | in vivo | in vivo | CXCL12 | | | |
|---|---|---|---|---|---|---|---|---|---|
| Compound | Dose | ALT | P21 | Tnfrsf10b | Gadd45a | mRNA | ED50 | MTND | Therapeutic |
| ID | (mg/kg) | (IU/L) | (% control) | (% control) | (% control) | (% control) | (mg/kg) | (mg/kg) | Index |
| 558807 | 1.8 | 24 | 118 | 120 | 147 | 67.5 | 2.75 | 5.5 | 2 |
| | 5.5 | 27 | 63 | 103 | 176 | 20.3 | | | |
| | 16.7 | 586 | 625 | 788 | 879 | 9.7 | | | |
| | 50 | death | n.d. | n.d. | n.d. | n.d. | | | |
| 936053 | 1.8 | 34 | 104 | 78 | 61 | 65.3 | 4.86 | 200 | 41 |
| | 5.5 | 26 | 94 | 137 | 99 | 47.4 | | | |
| | 16.7 | 23 | 104 | 110 | 91 | 32.7 | | | |
| | 50 | 23 | 89 | 122 | 90 | 14.4 | | | |
| | 100 | 42 | n.d. | n.d. | n.d. | n.d. | | | |
| | 200 | 109 | n.d. | n.d. | n.d. | n.d. | | | |
| | 300 | 231 | n.d. | n.d. | n.d. | n.d. | | | |

### Example 49 Toxicity Improvement of Modified Oligonucleotides Targeted to FXI

BALB/c mice were administered 1.8, 5.5, 16.7, 50 or 150 mg/kg of modified oligonucleotide by subcutaneous injection. Each group contained 3 mice. A group of 4 mice was administered PBS as a control. Plasma levels of ALT were measured with a clinical chemistry analyzer and mRNA was isolated and analyzed as described in Example 1. Modified oligonucleotides are described in Example 18. Each pair of compounds, presented adjacent to each other in the table below, represents a compound with the motif kkk-d(10)-kkk (464xxx) and the same sequence with the motif kkk-d-m-d(8)-kkk (1133xxx). In instances where position 5 in the original sequence is a T, this nucleoside is a 2′-OMeU in the kkk-d-m-d(8)-kkk sequence.

TABLE 108
in vivo dose response

| | | | in vivo | | | | |
|---|---|---|---|---|---|---|---|
| Compound | Dose | ALT | P21 | FXI mRNA | ED50 | MTND | Therapeutic |
| ID | (mg/kg) | (IU/L) | (% control) | (% control) | (mg/kg) | (mg/kg) | Index |
| 464924 | 1.8 | 27 | 136 | 59.7 | 2.4 | 16.7 | 7.0 |
| | 5.5 | 27 | 144 | 20.4 | | | |
| | 16.7 | 31 | 167 | 2.5 | | | |
| | 50 | 646 | 551 | 0.4 | | | |
| | 150 | 4509 | 1160 | 0.4 | | | |
| 1133247 | 1.8 | 32 | 130 | 75.0 | 3.4 | >150 | >44 |
| | 5.5 | 30 | 67 | 29.3 | | | |
| | 16.7 | 30 | 94 | 5.4 | | | |
| | 50 | 37 | 123 | 1.9 | | | |
| | 150 | 53 | 304 | 1.4 | | | |
| 465172 | 1.8 | 26 | 131 | 73.5 | 6.7 | >150 | >22 |
| | 5.5 | 22 | 102 | 57.8 | | | |
| | 16.7 | 23 | 99 | 28.8 | | | |
| | 50 | 25 | 102 | 13.8 | | | |
| | 150 | 33 | 177 | 6.2 | | | |
| 1133326 | 1.8 | 25 | 51 | 81.1 | 16.3 | >150 | >9 |
| | 5.5 | 25 | 64 | 81.8 | | | |
| | 16.7 | 24 | 55 | 49.0 | | | |
| | 50 | 24 | 78 | 21.1 | | | |
| | 150 | 22 | 90 | 11.8 | | | |
| 465174 | 1.8 | 25 | 192 | 67.5 | 4.2 | >150 | >36 |
| | 5.5 | 29 | 172 | 46.8 | | | |
| | 16.7 | 22 | 31 | 18.0 | | | |
| | 50 | 20 | 49 | 7.5 | | | |
| | 150 | 29 | 83 | 5.7 | | | |
| 1133328 | 1.8 | 21 | 40 | 74.8 | 4.8 | >150 | >32 |
| | 5.5 | 23 | 38 | 44.3 | | | |
| | 16.7 | 28 | 42 | 18.6 | | | |
| | 50 | 26 | 25 | 13.0 | | | |
| | 150 | 31 | 38 | 10.7 | | | |
| 465178 | 1.8 | 26 | 43 | 47.2 | 1.7 | 16.7 | 10 |
| | 5.5 | 35 | 119 | 18.4 | | | |
| | 16.7 | 73 | 627 | 4.3 | | | |
| | 50 | 1067 | 3509 | 0.7 | | | |
| | 150 | 11596 | 4849 | 0.4 | | | |
| 1133332 | 1.8 | 23 | 101 | 47.8 | 1.8 | 150 | 83 |
| | 5.5 | 35 | 42 | 30.7 | | | |
| | 16.7 | 33 | 136 | 13.1 | | | |
| | 50 | 41 | 600 | 3.7 | | | |
| | 150 | 117 | 1414 | 1.3 | | | |

### Example 50 Toxicity Improvement of Modified Oligonucleotides Targeted to HDAC2

BALB/c mice were administered 1.8, 5.5, 16.7, 50 or 150 mg/kg of modified oligonucleotide by subcutaneous injection. Each group contained 3 mice. A group of 4 mice was administered PBS as a control. Plasma levels of ALT were measured with a clinical chemistry analyzer and mRNA was isolated and analyzed as described in Example 1. Modified oligonucleotides are described in Example 19. Each pair of compounds, presented adjacent to each other in the table below, represents a compound with the motif kkk-d(10)-kkk (546xxx) and the same sequence with the motif kkk-d-m-d(8)-kkk (1133xxx). In instances where position 5 in the original sequence is a T, this nucleoside is a 2′-OMeU in the kkk-d-m-d(8)-kkk sequence.

TABLE 109
in vivo dose response

| Compound | Dose | ALT | HDAC2 mRNA | ED50 | MTND | Therapeutic |
|---|---|---|---|---|---|---|
| ID | (mg/kg) | (IU/L) | (% control) | (mg/kg) | (mg/kg) | Index |
| 546108 | 1.8 | 33 | 74 | 3.2 | 16.7 | 5.2 |
| | 5.5 | 47 | 26.2 | | | |
| | 16.7 | 168 | 3.8 | | | |
| | 50 | 1713 | 4.8 | | | |
| | 150 | 17844 | 4.5 | | | |
| 1133122 | 1.8 | 29 | 91.6 | 5.5 | >150 | >27 |
| | 5.5 | 25 | 48.3 | | | |
| | 16.7 | 2 | 11.0 | | | |
| | 50 | 43 | 1.1 | | | |
| | 150 | 78 | 1.1 | | | |
| 546110 | 1.8 | 25 | 72.9 | 6.4 | 16.7 | 2.6 |
| | 5.5 | 27 | 57.4 | | | |
| | 16.7 | 37 | 29.4 | | | |
| | 50 | 416 | 6.7 | | | |
| | 150 | 2817 | 6.0 | | | |
| 1133123 | 1.8 | 24 | 71.0 | 6.8 | >150 | >22 |
| | 5.5 | 80 | 49.6 | | | |
| | 16.7 | 25 | 47.8 | | | |
| | 50 | 25 | 8.0 | | | |
| | 150 | 28 | 3.0 | | | |
| 546118 | 1.8 | 30 | 69.9 | 23.8 | 16.7 | 0.7 |
| | 5.5 | 29 | 70.1 | | | |
| | 16.7 | 40 | 50.8 | | | |
| | 50 | 365 | 39.1 | | | |
| | 150 | 1681 | 36.0 | | | |
| 1133127 | 1.8 | 35 | 77.2 | 24.9 | >150 | >6 |
| | 5.5 | 25 | 60.6 | | | |
| | 16.7 | 26 | 57.1 | | | |
| | 50 | 25 | 39.9 | | | |
| | 150 | 33 | 34.4 | | | |

### Example 51 Toxicity Improvement of Modified Oligonucleotides

Modified oligonucleotides were tested for toxicity in vivo in Balb/c mice. Compound 865060 has the motif kkk-d(10)-kkkk and compound 865061 has the motif kkkk-d(10)-kkk. Compounds 1269430 and 1269431 are otherwise identical compounds to 865060 and 865061, respectively, containing a 2′-OMe modified sugar moiety at the second position in the central region.

TABLE 110
Modified Oligonucleotides

| Compound | | SEQ ID |
|---|---|---|
| ID | Chemistry notation | NO: |
| 865060 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖₛTₖ | 125 |
| 865061 | GₖₛGₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 141 |
| 1269430 | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖₛTₖ | 125 |
| 1269431 | GₖₛGₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | 141 |

A subscript “d” indicates an unmodified, 2′-β-D-deoxyribosyl sugar moiety. A subscript “k” indicates a cEt. A subscript “m” indicates a 2′-O-methyl-β-D-ribofuranosyl sugar moiety. A subscript “s” indicates a phosphorothioate internucleoside linkage. A superscript “m” before a C indicates 5-methyl Cytosine.

For in vivo toxicity studies, 3 BALB/c mice per group were administered the indicated dose of modified oligonucleotide by subcutaneous injection and sacrificed after 72 hours. Four animals were administered saline to serve as a control. RT-PCR was performed as described in Example 1 to determine mRNA levels of CXCL12, P21, Tnfrsf10b, and Gadd45a. Plasma levels of ALT was measured using an automated clinical chemistry analyzer. Increased ALT is indicative of acute liver toxicity.

For the in vitro toxicity study in the table below, the caspase assay was performed essentially as described in Example 8 in 3T3-L1 cells, the percent nucleolar p54nrb was visualized as described in Example 11, and the p21 mRNA levels were determined as described in Example 11.

TABLE 111
In vivo and in vitro toxicity of modified oligonucleotides

| | | Relative | in vitro | | | | |
|---|---|---|---|---|---|---|---|
| | in vitro | Caspase | p21 mRNA | in vitro | in vivo | in vivo | |
| | CXCL12 | Activation | (% Control) | % | p21 | Tnfrsf10b | ALT @ |
| Compound | IC₅₀ | (% Control) | @20 | nucleolar | @150 | mRNA @ | 150 mg/kg |
| ID | (nM) | @20 μM | μM | p54nrb | mg/kg | 150 mg/kg | (IU/L) |
| 558807 | 183 | 432 | 285 | 82 | 7152* | 5504* | 9928* |
| 936053 | 259 | 114 | 114 | 0 | 166 | 204 | 12 |
| 865060 | 310 | 355 | 224 | 64 | 7604* | 9339* | 11058* |
| 1269430 | 308 | 110 | 127 | 0 | 209 | 350 | 10 |
| 865061 | 510 | 738 | 198 | 75 | 12531 | 6351 | 9014 |
| 1269431 | 849 | 116 | 134 | 0 | 376 | 661 | 52 |

*Value at 50 mg/kg dose; mice administered 150 mg/kg did not survive

### Example 52 Long-Term Toxicity Improvement of Modified Oligonucleotides

Modified oligonucleotides were tested for toxicity in vivo in Balb/c mice. Mice were administered 50 mg/kg modified oligonucleotide once a week for six weeks and ALT was measured using an automated clinical chemistry analyzer. Compounds in the table below are described in Example 13. Each pair of compounds represents an oligonucleotide with the sugar motif kkk-d(10)-kkk (upper) and an oligonucleotide with the same sequence having the sugar motif kkk-d-m-d(8)-kkk (lower).

TABLE 112
Long-term Toxicity

| | Week of Dosing | | | | | |
|---|---|---|---|---|---|---|
| Compound | 1 | 2 | 3 | 4 | 5 | 6 |
| ID | ALT (IU/L) | | | | | |
| PBS | 35 | 65 | 27 | 45 | 55 | 23 |
| 572912 | 398 | 1036 | 2667 | 2787 | n.d. | n.d. |
| 1200898 | 36 | 53 | 70 | 112 | 187 | 513 |
| 797793 | 1210 | 972 | 1674 | 2703 | 3831 | 3635 |
| 1201073 | 99 | 143 | 165 | 178 | 217 | 330 |
| 576095 | 46 | 83 | 1017 | 1763 | 2886 | 4118 |
| 1200899 | 50 | 57 | 129 | 550 | 1225 | 1392 |

n.d. indicates that the mice were sacrificed prior to the measurement date.

### Example 53

Modified oligonucleotides were designed based on 546118. Each of the modified oligonucleotides is conjugated with a THA-GalNAc conjugate group at the 3′-end. THA-GalNac refers to this structure:

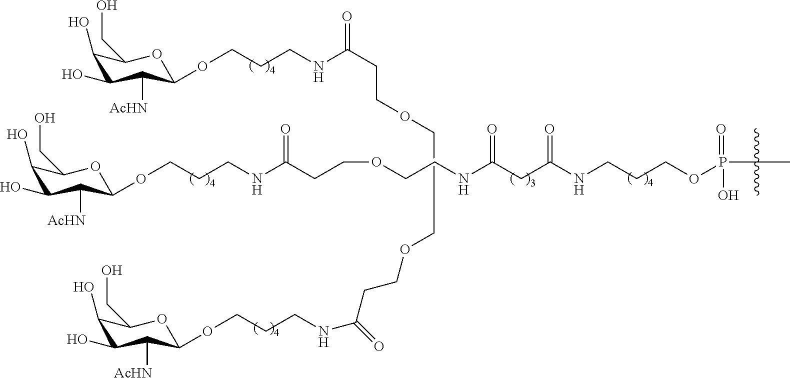

wherein the phosphate group is attached to the 3′-oxygen atom of the 3′ nucleoside.

The oligonucleotides were evaluated for reduction in HDAC2 mRNA expression levels in vivo. P21 and Tnfrsf10b mRNA levels were also measured. The transaminase levels (ALT and AST) for each dose were also measured.

Six week old BALB/C mice (purchased from Charles River) were injected subcutaneously once at dosage 0.023, 0.067, 0.2, 0.6, 1.8, 5.4, 15, or 50 mg/kg with the modified oligonucleotides shown below or with saline control. Each treatment group consisted of 3 animals. The mice were sacrificed 72 hours following administration, and organs and plasma were harvested for further analysis.

TABLE 113
Modified Oligonucleotides

| | position of | | | |
|---|---|---|---|---|
| | altered | | | |
| | nucleotide | modification | | |
| Compound | in central | of altered | | SEQ ID |
| ID | region | nucleotide | Chemistry Notation | NO |
| 1270732 | N/A | N/A | Aₖₛ^{m}Cₖₛ^{m}Cₖₛ^{m}C_{ds}T_{ds}^{m}C_{ds}A_{ds}A_{ds}G_{ds}T_{ds}^{m}C_{ds}T_{ds}^{m}Cₖₛ^{m}CₖₛTₖₛ | 109 |
| | | | Gₖ-THAGalNAc | |
| 1270733 | 2 | 2′-OMe | Aₖₛ^{m}Cₖₛ^{m}Cₖₛ^{m}C_{ds}Uₘₛ^{m}C_{ds}A_{ds}A_{ds}G_{ds}T_{ds}^{m}C_{ds}T_{ds}^{m}C_{ds}^{m}CₖₛTₖₛ | 140 |
| | | | Gₖ-THAGalNAc | |
| 1270734 | 2 | MOP | Aₖₛ^{m}Cₖₛ^{m}Cₖₛ^{m}C_{ds}T_{dx}^{m}C_{ds}A_{ds}A_{ds}G_{ds}T_{ds}^{m}C_{ds}T_{ds}^{m}C_{ds}^{m}CₖₛTₖₛ | 109 |
| | | | Gₖ-THAGalNAc | |

A subscript “d” indicates an unmodified, 2′-β-D-deoxyribosyl sugar moiety. A subscript “in” indicates a 2′-O-methyl-β-D-ribofuranosyl sugar moiety. A subscript “k” indicates a cEt. A subscript “s” indicates a phosphorothioate internucleoside linkage. A superscript “m” before a C indicates 5-methyl Cytosine. A subscript “x” indicates a MOP internucleoside linkage.

TABLE 114
In vivo Activity and Toxicity of Modified Oligonucleotides

| | | | | Tnfrsf10b | HDAC2 |
|---|---|---|---|---|---|
| Compound | Dose | ALT | P21 mRNA | mRNA | ED50 |
| ID | (mg/kg) | (IU/L) | (% Control) | (% Control) | (mg/kg) |
| 1270732 | 0.023 | 34 | 106 | n.d. | 0.060 |
| | 0.067 | 7 | 74 | n.d. | |
| | 0.2 | 15 | 113 | 80 | |
| | 0.6 | 13 | 112 | 76 | |
| | 1.8 | 33 | 537 | 118 | |
| | 5.4 | 122 | 688 | 271 | |
| | 15 | 1467 | 2606 | 1418 | |
| | 50 | 3429 | 5197 | 3064 | |
| 1270733 | 0.023 | 22 | 71 | n.d. | 0.066 |
| | 0.067 | 43 | 91 | n.d. | |
| | 0.2 | 18 | 89 | 80 | |
| | 0.6 | 20 | 103 | 104 | |
| | 1.8 | 17 | 81 | 81 | |
| | 5.4 | 23 | 154 | 81 | |
| | 15 | 11 | 172 | 110 | |
| | 50 | 22 | 988 | 353 | |
| 1270734 | 0.023 | 13 | 64 | n.d. | 0.084 |
| | 0.067 | 22 | 65 | n.d. | |
| | 0.2 | 31 | 158 | 93 | |
| | 0.6 | 7 | 230 | 149 | |
| | 1.8 | 12 | 64 | 93 | |
| | 5.4 | 20 | 169 | 110 | |
| | 15 | 318 | 1513 | 608 | |
| | 50 | 1650 | 2894 | 1368 | |

n.d. means a value was not determined.

### Example 54

Modified oligonucleotides were designed based on 546110, described in Example 19 above. Each of the modified oligonucleotides is conjugated with a THA-GalNAc conjugate group at the 3′-end as described in Example 53 above. The oligonucleotides were evaluated for reduction in HDAC2 mRNA expression levels in vivo. P21 and Tnfrsf10b mRNA levels were also measured. The transaminase levels (ALT and AST) for each dose were also measured.

Six week old BALB/C mice (purchased from Charles River) were injected subcutaneously once at dosage 0.016, 0.08, 0.40, 2.0, or 50 mg/kg with the modified oligonucleotides shown below or with saline control. Each treatment group consisted of 3 animals. The mice were sacrificed 72 hours following administration, and organs and plasma were harvested for further analysis.

TABLE 115
Modified Oligonucleotides

| | position of | | | |
|---|---|---|---|---|
| | altered | | | |
| | nucleotide | modification | | SEQ |
| Compound | in central | of altered | Chemistry Notation | ID |
| ID | region | nucleotide | (5′ to 3′) | NO |
| 1270729 | n/a | n/a | Tₖₛ^{m}CₖₛAₖₛT_{ds}G_{ds}T_{ds}A_{ds}^{m}C_{ds}^{m}C_{ds}T_{ds}A_{ds}T_{ds}A_{ds}GₖₛTₖₛ^{m}Cₖ- | 107 |
| | | | THA-GalNAc | |
| 1270733 | 2 | 2′-OMe | Tₖₛ^{m}CₖₛAₖₛT_{ds}GₘₛT_{ds}A_{ds}^{m}C_{ds}^{m}C_{ds}T_{ds}A_{ds}T_{ds}A_{ds}GₖₛTₖₛ^{m}Cₖ- | 107 |
| | | | THA-GalNAc | |
| 1270734 | 2 | MOP | Tₖₛ^{m}CₖₛAₖₛT_{ds}G_{dx}T_{ds}A_{ds}^{m}C_{ds}^{m}C_{ds}T_{ds}A_{ds}T_{ds}A_{ds}GₖₛTₖₛ^{m}Cₖ- | 107 |
| | | | THA-GalNAc | |

A subscript “d” indicates an unmodified, 2-β-D-deoxyribosyl sugar moiety. A subscript “k” indicates a cEt. A subscript “m” indicates a 2′-O-methyl-β-D-ribofuranosyl sugar moiety. A subscript “s” indicates a phosphorothioate internucleoside linkage. A superscript “m” before a C indicates 5-methyl Cytosine. A subscript “x” indicates a MOP internucleoside linkage.

TABLE 116
In vivo Activity and Toxicity of Modified Oligonucleotides

| | | | | Tnfrsf10b | HDAC2 |
|---|---|---|---|---|---|
| Compound | Dose | ALT | P21 mRNA | mRNA | ED50 |
| ID | (mg/kg) | (IU/L) | (% Control) | (% Control) | (mg/kg) |
| 1270729 | 0.016 | 6 | 69 | 123 | 0.76 |
| | 0.08 | 14 | 61 | 136 | |
| | 0.40 | 13 | 71 | 142 | |
| | 2.0 | 17 | 174 | 154 | |
| | 50 | 3655 | 7927 | 5297 | |
| 1270730 | 0.016 | 31 | 107 | 124 | 1.05 |
| | 0.08 | 10 | 144 | 132 | |
| | 0.40 | 17 | 65 | 99 | |
| | 2.0 | 9 | 88 | 123 | |
| | 50 | 11 | 110 | 164 | |
| 1270731 | 0.016 | 22 | 88 | 135 | 1.28 |
| | 0.08 | 13 | 86 | 101 | |
| | 0.40 | 20 | 135 | 138 | |
| | 2.0 | 13 | 66 | 137 | |
| | 50 | 6 | 76 | 164 | |

n.d. means a value was not determined.

### Example 55

Modified oligonucleotides were designed based on 747149, described in Example 13 above. Each of the modified oligonucleotides is conjugated with a THA-GalNAc conjugate group at the 3′-end as described in Example 53 above. The oligonucleotides were evaluated for reduction in FBO1A mRNA expression levels in vivo. P21 and Tnfrsf10b mRNA levels were also measured. The transaminase levels (ALT and AST) for each dose were also measured.

Six week old BALB/C mice (purchased from Charles River) were injected subcutaneously once at dosage 0.025, 0.10, 0.40, 1.6, 6.4, or 50 mg/kg with the modified oligonucleotides shown below or with saline control. Each treatment group consisted of 3 animals. The mice were sacrificed 72 hours following administration, and organs and plasma were harvested for further analysis.

TABLE 117
Modified Oligonucleotides

| | position of | | | |
|---|---|---|---|---|
| | altered | | | |
| | nucleotide | modification | | SEQ |
| Compound | in central | of altered | Chemistry Notation | ID |
| ID | region | nucleotide | (5′ to 3′) | NO |
| 1270738 | N/A | N/A | GₖₛGₖₛAₖₛ^{m}C_{ds}T_{ds}G_{ds}A_{ds}A_{ds}A_{ds}T_{ds}A_{ds}G_{ds}^{m}C_{ds}AₖₛGₖₛAₖ- | 54 |
| | | | THA-GalNAc | |
| 1270739 | 2 | 2′-OMe | GₖₛGₖₛAₖₛ^{m}C_{ds}UₘₛG_{ds}A_{ds}A_{ds}A_{ds}T_{ds}A_{ds}G_{ds}^{m}C_{ds}AₖₛGₖₛAₖ- | 130 |
| | | | THA-GalNAc | |
| 1270740 | 2 | MOP | GₖₛGₖₛAₖₛ^{m}C_{ds}T_{dx}G_{ds}A_{ds}A_{ds}A_{ds}T_{ds}A_{ds}G_{ds}^{m}C_{ds}AₖₛGₖₛAₖ- | 54 |
| | | | THA-GalNAc | |

A subscript “d” indicates an unmodified, 2′-β-D-deoxyribosyl sugar moiety. A subscript “k” indicates a cEt. A subscript “s” indicates a phosphorothioate internucleoside linkage. A superscript “m” before a C indicates 5-methyl Cytosine. A subscript “x” indicates a MOP internucleoside linkage. A subscript “m” indicates a 2′-O-methyl-β-D-ribofuranosyl sugar moiety.

TABLE 118
In vivo Activity and Toxicity of Modified Oligonucleotides

| | | | | Tnfrsf10b | FBO1A |
|---|---|---|---|---|---|
| Compound | Dose | ALT | P21 mRNA | mRNA | ED50 |
| ID | (mg/kg) | (IU/L) | (% Control) | (% Control) | (mg/kg) |
| 1270738 | 0.025 | 29 | 49 | 114 | 1.23 |
| | 0.10 | 18 | 46 | 119 | |
| | 0.40 | 30 | 156 | 133 | |
| | 1.6 | 62 | 273 | 258 | |
| | 6.4 | 177 | 1020 | 1426 | |
| | 50 | 1467 | 4296 | 10211 | |
| 1270739 | 0.025 | 10 | 66 | 115 | 5.16 |
| | 0.10 | 14 | 54 | 120 | |
| | 0.40 | 9 | 39 | 93 | |
| | 1.6 | 16 | 34 | 98 | |
| | 6.4 | 12 | 88 | 116 | |
| | 50 | 26 | 163 | 115 | |
| 1270740 | 0.025 | 25 | 59 | 94 | 3.33 |
| | 0.10 | 20 | 79 | 143 | |
| | 0.40 | 22 | 81 | 110 | |
| | 1.6 | 7 | 68 | 146 | |
| | 6.4 | 27 | 195 | 165 | |
| | 50 | 102 | 1378 | 439 | |

### Example 56

Modified oligonucleotides were designed based on 464924, described in Example 18 above. Each of the modified oligonucleotides is conjugated with a THA-GalNAc conjugate group at the 3′-endas described in Example 53 above. The oligonucleotides were evaluated for reduction in FXI mRNA expression levels in vivo. P21 and Tnfrsf10b mRNA levels were also measured. The transaminase levels (ALT and AST) for each dose were also measured.

Six week old BALB/C mice (purchased from Charles River) were injected subcutaneously once at dosage 0.025, 0.10, 0.40, 1.6, 6.4, or 50 mg/kg with the modified oligonucleotides shown below or with saline control. Each treatment group consisted of 3 animals. The mice were sacrificed 72 hours following administration, and organs and plasma were harvested for further analysis.

TABLE 119
Modified Oligonucleotides

| | position | | | |
|---|---|---|---|---|
| | of altered | | | |
| | nucleotide | modification | | SEQ |
| Compound | in central | of altered | Chemistry Notation | ID |
| ID | region | nucleotide | (5′ to 3′) | NO |
| 1270735 | N/A | N/A | GₖₛTₖₛTₖₛA_{ds}T_{ds}T_{ds}G_{ds}T_{ds}G_{ds}G_{ds}T_{ds}T_{ds}G_{ds}Gₖₛ^{m}CₖₛGₖ- | 81 |
| | | | THA-GalNAc | |
| 1270736 | 2 | 2′-OMe | GₖₛTₖₛTₖₛA_{ds}UₘₛT_{ds}G_{ds}T_{ds}G_{ds}G_{ds}T_{ds}T_{ds}G_{ds}Gₖₛ^{m}CₖₛGₖ- | 133 |
| | | | THA-GalNAc | |
| 1270737 | 2 | MOP | GₖₛTₖₛTₖₛA_{ds}T_{dx}T_{ds}G_{ds}T_{ds}G_{ds}G_{ds}T_{ds}T_{ds}G_{ds}Gₖₛ^{m}CₖₛGₖ- | 81 |
| | | | THA-GalNAc | |

A subscript “d” indicates an unmodified, 2′-β-D-deoxyribosyl sugar moiety. A subscript “k” indicates a cEt. A subscript “m” indicates a 2′-O-methyl-β-D-ribofuranosyl sugar moiety. A subscript “s” indicates a phosphorothioate internucleoside linkage. A superscript “m” before a C indicates 5-methyl Cytosine. A subscript “x” indicates a MOP internucleoside linkage.

TABLE 120
In vivo Activity and Toxicity of Modified Oligonucleotides

| | | | | Tnfrsf10b | FBO1A |
|---|---|---|---|---|---|
| Compound | Dose | ALT | P21 mRNA | mRNA | ED50 |
| ID | (mg/kg) | (IU/L) | (% Control) | (% Control) | (mg/kg) |
| 1270735 | 0.025 | 9 | 86 | 93 | 0.86 |
| | 0.10 | 8 | 26 | 66 | |
| | 0.40 | 10 | 94 | 81 | |
| | 1.6 | 22 | 69 | 95 | |
| | 6.4 | 3 | 114 | 137 | |
| | 50 | 30 | 266 | 308 | |
| 1270736 | 0.025 | 17 | 95 | 70 | 0.81 |
| | 0.10 | 26 | 53 | 65 | |
| | 0.40 | 29 | 77 | 58 | |
| | 1.6 | 11 | 53 | 93 | |
| | 6.4 | 12 | 64 | 90 | |
| | 50 | 28 | 92 | 125 | |
| 1270740 | 0.025 | 17 | 63 | 77 | 1.47 |
| | 0.10 | 14 | 83 | 101 | |
| | 0.40 | 9 | 62 | 72 | |
| | 1.6 | 21 | 98 | 105 | |
| | 6.4 | 12 | 33 | 104 | |
| | 50 | 11 | 168 | 214 | |

### Example 57 Introduction of 5′-Alkyl Modifications In Vivo

Modified oligonucleotides containing a 5′-alkyl modified nucleoside in the central region were synthesized.

The oligonucleotides were evaluated for reduction in target mRNA expression levels in vivo. The transaminase levels (ALT and AST) for each dose were measured.

For the in vitro toxicity study in the table below, the caspase assay was performed essentially as described in Example 8 in Hepa1-6 cells.

Fr the in vivo toxicity and activity study in the table below, six week old BALB/C mice (purchased from Charles River) were injected subcutaneously once at dosage 1.8, 5.5, 16.7, 50, or 150 mg/kg with the modified oligonucleotides shown below or with saline control. Each treatment group consisted of 3 animals. The mice were sacrificed 72 hours following administration, and organs and plasma were harvested for further analysis.

TABLE 121
Modified Oligonucleotides

| | | SEQ |
|---|---|---|
| Compound | | ID |
| ID | Chemistry notation | NO: |
| 546108 | TₖₛAₖₛGₖₛT_{ds}^{m}C_{ds}T_{ds}^{m}C_{ds}T_{ds}G_{ds}T_{ds}^{m}C_{ds}A_{ds}G_{ds}TₖₛTₖₛAₖ | 105 |
| 1133122 | TₖₛAₖₛGₖₛT_{ds}CₘₛT_{ds}^{m}C_{ds}T_{ds}G_{ds}T_{ds}^{m}C_{ds}A_{ds}G_{ds}TₖₛTₖₛAₖ | 105 |
| 1280765 | TₖₛAₖₛGₖₛT_{ds}^{m}C_{dx}T_{ds}^{m}C_{ds}T_{ds}G_{ds}T_{ds}^{m}C_{ds}A_{ds}G_{ds}TₖₛTₖₛAₖ | 105 |
| 1280766 | TₖₛAₖₛGₖₛT_{ds}^{m}C_{ds}T_{[(R)-μ]s}^{m}C_{ds}T_{ds}G_{ds}T_{ds}^{m}C_{ds}A_{ds}G_{ds}TₖₛTₖₛAₖ | 105 |
| 1280767 | TₖₛAₖₛGₖₛT_{ds}^{m}C_{ds}T_{ds}^{m}C_{[(R)-μ]s}T_{ds}G_{ds}T_{ds}^{m}C_{ds}A_{ds}G_{ds}TₖₛTₖₛAₖ | 105 |
| 1280768 | TₖₛAₖₛGₖₛT_{ds}^{m}C_{ds}T_{[(R)-ε]s}^{m}C_{ds}T_{ds}G_{ds}T_{ds}^{m}C_{ds}A_{ds}G_{ds}TₖₛTₖₛAₖ | 105 |
| 1280769 | TₖₛAₖₛGₖₛT_{ds}^{m}C_{ds}T_{ds}^{m}C_{[(R)-ε]s}T_{ds}G_{ds}T_{ds}^{m}C_{ds}A_{ds}G_{ds}TₖₛTₖₛAₖ | 105 |
| 694804 | AₖₛGₖₛAₖₛ^{m}C_{ds}T_{ds}^{m}C_{ds}T_{ds}^{m}C_{ds}G_{ds}G_{ds}T_{ds}T_{ds}^{m}C_{ds}^{m}CₖₛGₖₛAₖ | 49 |
| 1202810 | AₖₛGₖₛAₖₛ^{m}C_{ds}Uₘₛ^{m}C_{ds}T_{ds}^{m}C_{ds}G_{ds}G_{ds}T_{ds}T_{ds}^{m}C_{ds}^{m}CₖₛGₖₛAₖ | 127 |
| 1280776 | AₖₛGₖₛAₖₛ^{m}C_{ds}T_{dx}^{m}C_{ds}T_{ds}^{m}C_{ds}G_{ds}G_{ds}T_{ds}T_{ds}^{m}C_{ds}^{m}CₖₛGₖₛAₖ | 49 |
| 1280785 | AₖₛGₖₛAₖₛ^{m}C_{ds}T_{ds}^{m}C_{[(R)-μ]s}T_{ds}^{m}C_{ds}G_{ds}G_{ds}T_{ds}T_{ds}^{m}C_{ds}^{m}CₖₛGₖₛAₖ | 49 |
| 1280795 | AₖₛGₖₛAₖₛ^{m}C_{ds}T_{ds}^{m}C_{ds}T_{[(R)-μ]s}^{m}C_{ds}G_{ds}G_{ds}T_{ds}T_{ds}^{m}C_{ds}^{m}CₖₛGₖₛAₖ | 49 |
| 1280804 | AₖₛGₖₛAₖₛ^{m}C_{ds}T_{ds}^{m}C_{[(R)-μ]s}T_{ds}^{m}C_{ds}G_{ds}G_{ds}T_{ds}T_{ds}^{m}C_{ds}^{m}CₖₛGₖₛAₖ | 49 |
| 1280810 | AₖₛGₖₛAₖₛ^{m}C_{ds}T_{ds}^{m}C_{ds}T_{[(R)-μ]s}^{m}C_{ds}G_{ds}G_{ds}T_{ds}T_{ds}^{m}C_{ds}^{m}CₖₛGₖₛAₖ | 49 |
| 465178 | GₖₛTₖₛ^{m}CₖₛA_{ds}G_{ds}T_{ds}A_{ds}T_{ds}^{m}C_{ds}^{m}C_{ds}^{m}C_{ds}A_{ds}G_{ds}TₖₛGₖₛTₖ | 89 |
| 1133332 | GₖₛTₖₛ^{m}CₖₛA_{ds}GₘₛT_{ds}A_{ds}T_{ds}^{m}C_{ds}^{m}C_{ds}^{m}C_{ds}A_{ds}G_{ds}TₖₛGₖₛTₖ | 89 |
| 1280775 | GₖₛTₖₛ^{m}CₖₛA_{ds}G_{dx}T_{ds}A_{ds}T_{ds}^{m}C_{ds}^{m}C_{ds}^{m}C_{ds}A_{ds}G_{ds}TₖₛGₖₛTₖ | 89 |
| 1280784 | GₖₛTₖₛ^{m}CₖₛA_{ds}G_{ds}T_{[(R)-μ]s}A_{ds}T_{ds}^{m}C_{ds}^{m}C_{ds}^{m}C_{ds}A_{ds}G_{ds}TₖₛGₖₛTₖ | 89 |
| 1280794 | GₖₛTₖₛ^{m}CₖₛA_{ds}G_{ds}T_{ds}A_{[(R)-μ]s}T_{ds}^{m}C_{ds}^{m}C_{ds}^{m}C_{ds}A_{ds}G_{ds}TₖₛGₖₛTₖ | 89 |
| 1280803 | GₖₛTₖₛ^{m}CₖₛA_{ds}G_{ds}T_{[(R)-ε]s}A_{ds}T_{ds}^{m}C_{ds}^{m}C_{ds}^{m}C_{ds}A_{ds}G_{ds}TₖₛGₖₛTₖ | 89 |
| 1281809 | GₖₛTₖₛ^{m}CₖₛA_{ds}G_{ds}T_{ds}A_{[(R)-ε]s}T_{ds}^{m}C_{ds}^{m}C_{ds}^{m}C_{ds}A_{ds}G_{ds}TₖₛGₖₛTₖ | 89 |
| 546110 | Tₖₛ^{m}CₖₛAₖₛT_{ds}G_{ds}T_{ds}A_{ds}^{m}C_{ds}^{m}C_{ds}T_{ds}A_{ds}T_{ds}A_{ds}GₖₛTₖₛ^{m}Cₖ | 107 |
| 1133201 | Tₖₛ^{m}CₖₛAₖₛT_{ds}GₘₛT_{ds}A_{ds}^{m}C_{ds}^{m}C_{ds}T_{ds}A_{ds}T_{ds}A_{ds}GₖₛTₖₛ^{m}Cₖ | 107 |
| 1280778 | Tₖₛ^{m}CₖₛAₖₛT_{ds}G_{dx}T_{ds}A_{ds}^{m}C_{ds}^{m}C_{ds}T_{ds}A_{ds}T_{ds}A_{ds}GₖₛTₖₛ^{m}Cₖ | 107 |
| 1280779 | Tₖₛ^{m}CₖₛAₖₛT_{ds}G_{ds}T_{[(R)-μ]s}A_{ds}^{m}C_{ds}^{m}C_{ds}T_{ds}A_{ds}T_{ds}A_{ds}GₖₛTₖₛ^{m}Cₖ | 107 |
| 1280789 | Tₖₛ^{m}CₖₛAₖₛT_{ds}G_{ds}T_{ds}A_{[(R)-μ]s}^{m}C_{ds}^{m}C_{ds}T_{ds}A_{ds}T_{ds}A_{ds}GₖₛTₖₛ^{m}Cₖ | 107 |
| 1280798 | Tₖₛ^{m}CₖₛAₖₛT_{ds}G_{ds}T_{[(R)-ε]s}A_{ds}^{m}C_{ds}^{m}C_{ds}T_{ds}A_{ds}T_{ds}A_{ds}GₖₛTₖₛ^{m}Cₖ | 107 |
| 1281804 | Tₖₛ^{m}CₖₛAₖₛT_{ds}G_{ds}T_{ds}A_{[(R)-ε]s}^{m}C_{ds}^{m}C_{ds}T_{ds}A_{ds}T_{ds}A_{ds}GₖₛTₖₛ^{m}Cₖ | 107 |
| 464924 | GₖₛTₖₛTₖₛA_{ds}T_{ds}T_{ds}G_{ds}T_{ds}G_{ds}G_{ds}T_{ds}T_{ds}G_{ds}Gₖₛ^{m}CₖₛGₖ | 81 |
| 1133247 | GₖₛTₖₛTₖₛA_{ds}UₘₛT_{ds}G_{ds}T_{ds}G_{ds}G_{ds}T_{ds}T_{ds}G_{ds}Gₖₛ^{m}CₖₛGₖ | 133 |
| 1280774 | GₖₛTₖₛTₖₛA_{ds}T_{dx}T_{ds}G_{ds}T_{ds}G_{ds}G_{ds}T_{ds}T_{ds}G_{ds}Gₖₛ^{m}CₖₛGₖ | 81 |
| 1280783 | GₖₛTₖₛTₖₛA_{ds}T_{ds}T_{[(R)-μ]s}G_{ds}T_{ds}G_{ds}G_{ds}T_{ds}T_{ds}G_{ds}Gₖₛ^{m}CₖₛGₖ | 81 |
| 1280793 | GₖₛTₖₛTₖₛA_{ds}T_{ds}T_{ds}G_{[(R)-μ]s}T_{ds}G_{ds}G_{ds}T_{ds}T_{ds}G_{ds}Gₖₛ^{m}CₖₛGₖ | 81 |
| 747149 | GₖₛGₖₛAₖₛ^{m}C_{ds}T_{ds}G_{ds}A_{ds}A_{ds}A_{ds}T_{ds}A_{ds}G_{ds}^{m}C_{ds}AₖₛGₖₛAₖ | 54 |
| 1203759 | GₖₛGₖₛAₖₛ^{m}C_{ds}UₘₛG_{ds}A_{ds}A_{ds}A_{ds}T_{ds}A_{ds}G_{ds}^{m}C_{ds}AₖₛGₖₛAₖ | 130 |
| 1280778 | GₖₛGₖₛAₖₛ^{m}C_{ds}T_{dx}G_{ds}A_{ds}A_{ds}A_{ds}T_{ds}A_{ds}G_{ds}^{m}C_{ds}AₖₛGₖₛAₖ | 54 |
| 1280787 | GₖₛGₖₛAₖₛ^{m}C_{ds}T_{ds}G_{[(R)-μ]s}A_{ds}A_{ds}A_{ds}T_{ds}A_{ds}G_{ds}^{m}C_{ds}AₖₛGₖₛAₖ | 54 |
| 1280797 | GₖₛGₖₛAₖₛ^{m}C_{ds}T_{ds}G_{ds}A_{[(R)-μ]s}A_{ds}A_{ds}T_{ds}A_{ds}G_{ds}^{m}C_{ds}AₖₛGₖₛAₖ | 54 |

A subscript “d” indicates an unmodified, 2-β-D-deoxyribosyl sugar moiety. A subscript “k” indicates a cEt. A subscript “s” indicates phosphorothioate internucleoside linkage. A subscript “[(R)-μ]” indicates a 5′-(R)-Me-β-D-2′-deoxyribosyl sugar moiety. A subscript “[(R)-ε]” indicates a 5′-(R)-ethyl-β-D-2′-deoxyribosyl sugar moiety.

TABLE 122
Activity and Toxicity in vitro and in vivo

| | | position | | | | Relative |
|---|---|---|---|---|---|---|
| | | of altered | | in vivo | | Caspase |
| | | nucleoside | modification | Target | in vivo | Activation |
| Compound | | in central | of altered | ED₅₀ | ALT | (% Control) |
| ID | Target | region | nucleoside | (mg/kg) | @150 mg/kg | @20 μM |
| 546108 | HDAC2 | N/A | N/A | n.d. | n.d. | 2436 |
| 1133122 | HDAC2 | 2 | 2′-OMe | 6.1 | 127 | 103 |
| 1280765 | HDAC2 | 2 | MOP | 7.7 | 29 | 157 |
| 1280766 | HDAC2 | 3 | 5′-(R)-Me | 4.5 | 61 | 158 |
| 1280767 | HDAC2 | 4 | 5′-(R)-Me | 5.9 | 79 | 122 |
| 1280768 | HDAC2 | 3 | 5′-(R)-Et | 8.6 | 39 | 70 |
| 1280769 | HDAC2 | 4 | 5′-(R)-Et | 9.6 | 55 | 90 |
| 694804 | DMN2 | N/A | N/A | n.d. | n.d. | 1443 |
| 1202810 | DMN2 | 2 | 2′-OMe | 14.7 | 50 | 70 |
| 1280776 | DMN2 | 2 | MOP | 8.6 | 24 | 214 |
| 1280785 | DMN2 | 3 | 5′-(R)-Me | 6.2 | 92 | 285 |
| 1280795 | DMN2 | 4 | 5′-(R)-Me | 7.3 | 27 | 113 |
| 1280804 | DMN2 | 3 | 5′-(R)-Et | 14.9 | 36 | 135 |
| 1280810 | DMN2 | 4 | 5′-(R)-Et | 16.2 | 26 | 211 |
| 465178 | FXI | N/A | N/A | n.d. | n.d. | 506 |
| 1133332 | FXI | 2 | 2′-OMe | 2.0 | 119 | 168 |
| 1280775 | FXI | 2 | MOP | 2.5 | 153 | 136 |
| 1280784 | FXI | 3 | 5′-(R)-Me | 1.7 | 260 | 145 |
| 1280794 | FXI | 4 | 5′-(R)-Me | 2.3 | 358 | 165 |
| 1280803 | FXI | 3 | 5′-(R)-Et | 4.9 | 122 | 104 |
| 1281809 | FXI | 4 | 5′-(R)-Et | 21.2 | 56 | 93 |
| 546110 | FXI | N/A | N/A | n.d. | n.d. | 404 |
| 1133201 | FXI | 2 | 2′-OMe | 10.6 | 127 | 96 |
| 1280778 | FXI | 2 | MOP | 26.6 | 29 | 105 |
| 1280779 | FXI | 3 | 5′-(R)-Me | 10.9 | 61 | 84 |
| 1280789 | FXI | 4 | 5′-(R)-Me | 11.7 | 79 | 74 |
| 1280798 | FXI | 3 | 5′-(R)-Et | 30.5 | 39 | 78 |
| 1281804 | FXI | 4 | 5′-(R)-Et | 31.5 | 55 | 80 |
| 464924 | FXI | N/A | N/A | n.d. | n.d. | n.d. |
| 1133247 | FXI | 2 | 2′-OMe | 4.6 | 51 | n.d. |
| 1280774 | FXI | 2 | MOP | 3.7 | 37 | n.d. |
| 1280783 | FXI | 3 | 5′-(R)-Me | 3.6 | 48 | n.d. |
| 1280793 | FXI | 4 | 5′-(R)-Me | 2.3 | 351 | n.d. |
| 747149 | FOXO1A | N/A | N/A | n.d. | n.d. | n.d. |
| 1203759 | FOXO1A | 2 | 2′-OMe | 24.9 | 30 | n.d. |
| 1280778 | FOXO1A | 2 | MOP | 8.5 | 35 | n.d. |
| 1280787 | FOXO1A | 3 | 5′-(R)-Me | 65.9 | 62 | n.d. |
| 1280797 | FOXO1A | 4 | 5′-(R)-Me | 20.4 | 22 | n.d. |

### Example 58 Nucleosides with Chiral Phosphorothioate Linkages

Modified oligonucleotides containing chirally-controlled phosphorothioate linkages in the central region were synthesized. The compounds in the table below are 100% complementary to mouse CXCL12, GENBANK NT_039353.7 truncated from 69430515 to 69445350 (SEQ ID NO: 1), at position 6877 to 6892. Each compound has the kkk-d(10)-kkk sugar motif, wherein each “k” represents a 2′-constrained ethyl modified sugar moiety and each “d” represents a 2′-deoxy sugar moiety. Internucleoside linkages 1, 2, 3, 14, and 15 are stereorandom phosphorothioate linkages. Internucleoside linkages 4-13 have the stereochemistry indicated in the table below, wherein a subscript “s” indicates a stereorandom phosphorothioate internucleoside linkage, a subscript “r” indicates a phosphorothioate internucleoside linkage having the (Rp) configuration and a subscript “q” indicates a phosphorothioate internucleoside linkage having the (Sp) configuration.

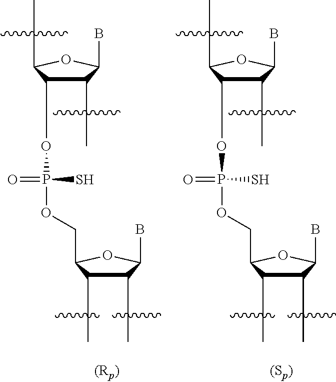

TABLE 123
modified oligonucleotides with stereochemically-controlled
phosphorothioate linkages

| | | SEQ |
|---|---|---|
| Compound | | ID |
| ID | Chemistry Notation | NO |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 1220041 | Gₖₛ^{m}CₖₛAₖₛT_{dq}G_{dq}T_{dq}T_{dq}^{m}C_{dq}T_{dq}^{m}C_{dq}A_{dq}^{m}D_{dq}A_{dq}TₖₛTₖₛAₖ | 18 |
| 1220042 | Gₖₛ^{m}CₖₛAₖₛT_{dr}G_{dq}T_{dq}T_{dq}^{m}C_{dq}T_{dq}^{m}D_{dq}A_{dq}^{m}D_{dq}A_{dq}TₖₛTₖₛAₖ | 18 |
| 1220043 | Gₖₛ^{m}CₖₛAₖₛT_{dq}G_{dr}T_{dq}T_{dq}^{m}C_{dq}T_{dq}^{m}D_{dq}A_{dq}^{m}C_{dq}A_{dq}TₖₛTₖₛAₖ | 18 |
| 1220044 | Gₖₛ^{m}CₖₛAₖₛT_{dq}G_{dq}T_{dr}T_{dq}^{m}C_{dq}T_{dq}^{m}D_{dq}A_{dq}^{m}C_{dq}A_{dq}TₖₛTₖₛAₖ | 18 |
| 1220045 | Gₖₛ^{m}CₖₛAₖₛT_{dq}G_{dq}T_{dq}T_{dr}^{m}C_{dq}T_{dq}^{m}C_{dq}A_{dq}^{m}D_{dq}A_{dq}TₖₛTₖₛAₖ | 18 |
| 1220046 | Gₖₛ^{m}CₖₛAₖₛT_{dq}G_{dq}T_{dq}T_{dq}^{m}C_{dr}T_{dq}^{m}C_{dq}A_{dq}^{m}D_{dq}A_{dq}TₖₛTₖₛAₖ | 18 |
| 1220051 | Gₖₛ^{m}CₖₛAₖₛT_{dq}G_{dq}T_{dq}T_{dq}^{m}C_{dq}T_{dr}^{m}C_{dq}A_{dq}^{m}C_{dq}A_{dq}TₖₛTₖₛAₖ | 18 |
| 1220047 | Gₖₛ^{m}CₖₛAₖₛT_{dq}G_{dq}T_{dq}T_{dq}^{m}C_{dq}T_{dq}^{m}C_{dr}A_{dq}^{m}C_{dq}A_{dq}TₖₛTₖₛAₖ | 18 |
| 1220048 | Gₖₛ^{m}CₖₛAₖₛT_{dq}G_{dq}T_{dq}T_{dq}^{m}C_{dq}T_{dq}^{m}C_{dq}A_{dr}^{m}D_{dq}A_{dq}TₖₛTₖₛAₖ | 18 |
| 1220049 | Gₖₛ^{m}CₖₛAₖₛT_{dq}G_{dq}T_{dq}T_{dq}^{m}C_{dq}T_{dq}^{m}C_{dq}A_{dq}^{m}C_{dr}A_{dq}TₖₛTₖₛAₖ | 18 |
| 1220050 | Gₖₛ^{m}CₖₛAₖₛT_{dq}G_{dq}T_{dq}T_{dq}^{m}C_{dq}T_{dq}^{m}C_{dq}A_{dq}^{m}C_{dq}A_{dr}TₖₛTₖₛAₖ | 18 |
| 1237987 | Gₖₛ^{m}CₖₛAₖₛT_{dq}G_{dq}T_{dq}T_{dq}^{m}C_{dq}T_{dq}^{m}C_{dq}A_{dq}^{m}C_{dr}A_{dr}TₖₛTₖₛAₖ | 18 |
| 1237988 | Gₖₛ^{m}CₖₛAₖₛT_{dq}G_{dq}T_{dq}T_{dq}^{m}C_{dq}T_{dq}^{m}C_{dr}A_{dr}^{m}D_{dq}A_{dq}TₖₛTₖₛAₖ | 18 |
| 1237989 | Gₖₛ^{m}CₖₛAₖₛT_{dq}G_{dq}T_{dq}T_{dq}^{m}C_{dr}T_{dr}^{m}C_{dq}A_{dq}^{m}C_{dq}A_{dq}TₖₛTₖₛAₖ | 18 |
| 1237990 | Gₖₛ^{m}CₖₛAₖₛT_{dq}G_{dq}T_{dr}T_{dr}^{m}C_{dq}T_{dq}^{m}C_{dq}A_{dq}^{m}D_{dq}A_{dq}TₖₛTₖₛAₖ | 18 |
| 1237991 | Gₖₛ^{m}CₖₛAₖₛT_{dr}G_{dr}T_{dq}T_{dq}^{m}C_{dq}T_{dq}^{m}C_{dq}A_{dq}^{m}D_{dq}A_{dq}TₖₛTₖₛAₖ | 18 |
| 1220052 | Gₖₛ^{m}CₖₛAₖₛT_{dr}G_{dr}T_{dr}T_{dr}^{m}C_{dr}T_{dr}^{m}C_{dr}A_{dr}^{m}C_{dr}A_{dr}TₖₛTₖₛAₖ | 18 |
| 1220053 | Gₖₛ^{m}CₖₛAₖₛT_{dq}G_{dr}T_{dr}T_{dr}^{m}C_{dr}T_{dr}^{m}C_{dr}A_{dr}^{m}C_{dr}A_{dr}TₖₛTₖₛAₖ | 18 |
| 1220054 | Gₖₛ^{m}CₖₛAₖₛT_{dr}G_{dq}T_{dr}T_{dr}^{m}C_{dr}T_{dr}^{m}C_{dr}A_{dr}^{m}C_{dr}A_{dr}TₖₛTₖₛAₖ | 18 |
| 1220055 | Gₖₛ^{m}CₖₛAₖₛT_{dr}G_{dr}T_{dq}T_{dr}^{m}C_{dr}T_{dr}^{m}C_{dr}A_{dr}^{m}C_{dr}A_{dr}TₖₛTₖₛAₖ | 18 |
| 1220056 | Gₖₛ^{m}CₖₛAₖₛT_{dr}G_{dr}T_{dr}T_{dq}^{m}C_{dr}T_{dr}^{m}C_{dr}A_{dr}^{m}C_{dr}A_{dr}TₖₛTₖₛAₖ | 18 |
| 1220057 | Gₖₛ^{m}CₖₛAₖₛT_{dr}G_{dr}T_{dr}T_{dr}^{m}C_{dq}T_{dr}^{m}C_{dr}A_{dr}^{m}C_{dr}A_{dr}TₖₛTₖₛAₖ | 18 |
| 1220058 | Gₖₛ^{m}CₖₛAₖₛT_{dr}G_{dr}T_{dr}T_{dr}^{m}C_{dr}T_{dq}^{m}C_{dr}A_{dr}^{m}C_{dr}A_{dr}TₖₛTₖₛAₖ | 18 |
| 1220059 | Gₖₛ^{m}CₖₛAₖₛT_{dr}G_{dr}T_{dr}T_{dr}^{m}C_{dr}T_{dr}^{m}C_{dq}A_{dr}^{m}C_{dr}A_{dr}TₖₛTₖₛAₖ | 18 |
| 1220060 | Gₖₛ^{m}CₖₛAₖₛT_{dr}G_{dr}T_{dr}T_{dr}^{m}C_{dr}T_{dr}^{m}C_{dr}A_{dq}^{m}C_{dr}A_{dr}TₖₛTₖₛAₖ | 18 |
| 1220061 | Gₖₛ^{m}CₖₛAₖₛT_{dr}G_{dr}T_{dr}T_{dr}^{m}C_{dr}T_{dr}^{m}C_{dr}A_{dr}^{m}D_{dq}A_{dr}TₖₛTₖₛAₖ | 18 |
| 1220062 | Gₖₛ^{m}CₖₛAₖₛT_{dr}G_{dr}T_{dr}T_{dr}^{m}C_{dr}T_{dr}^{m}C_{dr}A_{dr}^{m}C_{dr}A_{dq}TₖₛTₖₛAₖ | 18 |
| 1220063 | Gₖₛ^{m}CₖₛAₖₛT_{dq}G_{dq}T_{dr}T_{dq}^{m}C_{dq}T_{dr}^{m}C_{dq}A_{dq}^{m}C_{dr}A_{dr}TₖₛTₖₛAₖ | 18 |
| 1220064 | Gₖₛ^{m}CₖₛAₖₛT_{dr}G_{dr}T_{dq}T_{dr}^{m}C_{dr}T_{dq}^{m}C_{dr}A_{dr}^{m}D_{dq}A_{dq}TₖₛTₖₛAₖ | 18 |

A subscript “d” an unmodified, 2′-β-D-deoxyribosyl sugar moiety. A subscript “k” indicates a cEt.. A superscript “m” before a C indicates 5-methyl Cytosine. A subscript “s” indicates a stereorandom phosphorothioate internucleoside linkage, a subscript “r” indicates a phosphorothioate internucleoside linkage having the (Rp) configuration, and a subscript “q” indicates a phosphorothioate internucleoside linkage having the (Sp) configuration.

For in vitro activity studies, 3T3-L1 cells were plated at 20,000 cells/well and transfected with 27 nM, 80 nM, 250 nM, 740 nM, 2,222 nM, 6,667 nM, or 20,000 nMnM modified oligonucleotide by electroporation. mRNA was harvested and analyzed by RT-qPCR. CXCL12 mRNA was detected with primer probe set RTS 2605 (forward sequence CCAGAGCCAACGTCAAGCAT, SEQ ID NO: 9; reverse sequence: CAGCCGTGCAACAATCTGAA, SEQ ID NO: 10; probe sequence: TGAAAATCCTCAACACTCCAAACTGTGCC, SEQ ID NO: 11) and P21 mRNA was detected with primer probe set Mm04207341_m1 (ThermoFisher).

Caspase-3 and caspase-7 activation was measured using the Caspase-Glo® 3/7 Assay System (G8090, Promega). Levels of caspase activation correlate with apoptotic cell death. Results are presented relative to the caspase activation in control cells not treated with modified oligonucleotide. Localization of p54nrb in HeLa cells was visualized with confocal microscopy. HeLa cells were transfected by lipofectamine 2000 with 200 nM of modified oligonucleotide for 2 hrs and then cellular protein p54nrb was stained by mP54 antibody (Santa Cruz Biotech, sc-376865) and DAPI was used to stain for the nucleus of cells. The number of cells with nucleolar p54nrb and the total number of cells in the images were counted. The self-structure Tm of each compound was determined.

TABLE 124
In vitro activity, toxicity, and Tm of modified
oligonucleotides complementary to CXCL12

| | in vitro | in vitro | | | |
|---|---|---|---|---|---|
| | CXCL12 | Caspase | P21 mRNA | % | |
| Compound | IC₅₀ | (% control) | (% control) | nucleolar | Tm |
| ID | (nM) | @ 20 μM | @ 20 μM | p54nrb | (° C.) |
| 558807 | 39 | 1437 | 353 | 90 | 64.4 |
| 1220041 | 388 | 223 | 182 | 0 | 61.3 |
| 1220042 | 159 | 584 | 431 | 32 | 62.1 |
| 1220043 | 114 | 838 | 488 | 88 | 62 |
| 1220044 | 181 | 489 | 251 | 18 | 61.5 |
| 1220045 | 222 | 321 | 259 | 9.7 | 61.9 |
| 1220046 | 145 | 572 | 635 | 28 | 61.7 |
| 1220051 | 237 | 310 | 167 | 20 | 61.6 |
| 1220047 | 60 | 814 | 238 | 38 | 61.5 |
| 1220048 | 74 | 287 | 174 | 38 | 61.3 |
| 1220049 | 77 | 323 | 243 | 17 | 61.6 |
| 1220050 | 132 | 174 | 121 | 6.4 | 61.5 |
| 1237987 | 26 | 317 | 273 | 3.9 | 62.2 |
| 1237988 | 20 | 336 | 236 | 23 | 62.1 |
| 1237989 | 72 | 300 | 394 | 28 | 62.2 |
| 1237990 | 186 | 299 | 355 | 14 | 62.5 |
| 1237991 | 35 | 562 | 585 | 77 | 63 |

TABLE 125
In vitro activity, toxicity, and Tm of modified
oligonucleotides complementary to CXCL12

| | in vitro | in vitro | | | |
|---|---|---|---|---|---|
| | CXCL12 | Caspase | P21 mRNA | % | |
| Compound | IC₅₀ | (% control) | (% control) | nucleolar | |
| ID | (nM) | (a), 20 μM | @ 20 μM | p54nrb | Tm |
| 558807 | 95 | 647 | 235 | 93 | 64.4 |
| 1220052 | 63 | 484 | 272 | 98 | 67.4 |
| 1220053 | 99 | 621 | 261 | 95 | 66.2 |
| 1220054 | 197 | 495 | 192 | 96 | 66.8 |
| 1220055 | 51 | 606 | 370 | 100 | 66.9 |
| 1220056 | 103 | 569 | 369 | 97 | 67 |
| 1220057 | 104 | 593 | 330 | 92 | 67.1 |
| 1220058 | 125 | 578 | 273 | 100 | 67.3 |
| 1220059 | 109 | 525 | 351 | 62 | 66.7 |
| 1220060 | 61 | 553 | 328 | 100 | 67.3 |
| 1220061 | 84 | 409 | 329 | 100 | 67.1 |
| 1220062 | 123 | 550 | 394 | 100 | 67.1 |
| 1220063 | 111 | 138 | 128 | 12 | 63.1 |
| 1220064 | 53 | 160 | 218 | 100 | 65.3 |

### Example 59 Nucleosides with Chiral Phosphorothioate Linkages and 3′-GalNAc

Modified oligonucleotides containing chirally-controlled phosphorothioate linkages in the central region and a 3′-THA-GalNAc were synthesized. The compounds in the table below are 100% complementary to mouse CXCL12, GENBANK NT_039353.7 truncated from 69430515 to 69445350 (SEQ ID NO: 1), at position 6877 to 6892. Each compound has the kkk-d(10)-kkk sugar motif, wherein each “k” represents a 2′-constrained ethyl modified sugar moiety and each “d” represents a 2′-deoxy sugar moiety. Internucleoside linkages 1, 2, 3, 14, and 15 are stereorandom phosphorothioate linkages. Internucleoside linkages 4-13 have the stereochemistry indicated in the table below, wherein a subscript “s” indicates a stereorandom phosphorothioate internucleoside linkage, a subscript “r” indicates a phosphorothioate internucleoside linkage having the (Rp) configuration and a subscript “q” indicates a phosphorothioate internucleoside linkage having the (Sp) configuration.

THA-GalNAc refers to this structure at the 3′ end of the molecule:

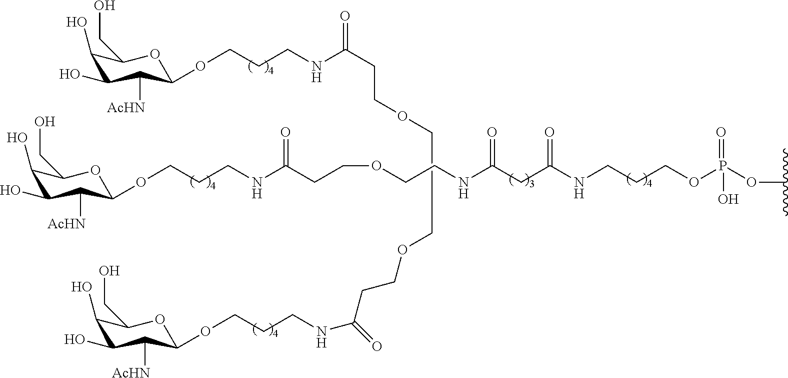

wherein the phosphate group is attached to the 3′-oxygen atom of the 3′ nucleoside.

TABLE 126
Modified oligonucleotides

| | | SEQ |
|---|---|---|
| Compound | | ID |
| ID | Chemistry Notation | NO |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 18 |
| 855156 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ-THA-GalNAc | 18 |
| 1220050 | Gₖₛ^{m}CₖₛAₖₛT_{dq}G_{dq}T_{dq}T_{dq}^{m}C_{dq}T_{dq}^{m}C_{dq}A_{dq}^{m}C_{dq}A_{dr}TₖₛTₖₛAₖ | 18 |
| 1277251 | Gₖₛ^{m}CₖₛAₖₛT_{dq}G_{dq}T_{dq}T_{dq}^{m}C_{dq}T_{dq}^{m}C_{dq}A_{dq}^{m}C_{dq}A_{dr}TₖₛTₖₛAₖ-THA-GalNAc | 18 |
| 1220059 | Gₖₛ^{m}CₖₛAₖₛT_{dr}G_{dr}T_{dr}T_{dr}^{m}C_{dr}T_{dr}^{m}C_{dq}A_{dr}^{m}C_{dr}A_{dr}TₖₛTₖₛAₖ | 18 |
| 1277252 | Gₖₛ^{m}CₖₛAₖₛT_{dr}G_{dr}T_{dr}T_{dr}^{m}C_{dr}T_{dr}^{m}D_{dq}A_{dr}^{m}C_{dr}A_{dr}TₖₛTₖₛAₖ-THA-GalNAc | 18 |
| 1220063 | Gₖₛ^{m}CₖₛAₖₛT_{dq}G_{dq}T_{dr}T_{dq}^{m}C_{dq}T_{dr}^{m}C_{dq}A_{dq}^{m}C_{dr}A_{dr}TₖₛTₖₛAₖ | 18 |
| 1277253 | Gₖₛ^{m}CₖₛAₖₛT_{dq}G_{dq}T_{dr}T_{dq}^{m}C_{dq}T_{dr}^{m}C_{dq}A_{dq}^{m}C_{dr}A_{dr}TₖₛTₖₛAₖ-THA-GalNAc | 18 |
| 1237988 | Gₖₛ^{m}CₖₛAₖₛT_{dq}G_{dq}T_{dq}T_{dq}^{m}C_{dq}T_{dq}^{m}C_{dr}A_{dr}^{m}C_{dq}A_{dr}TₖₛTₖₛAₖ | 18 |
| 1277254 | Gₖₛ^{m}CₖₛAₖₛT_{dq}G_{dq}T_{dq}T_{dq}^{m}C_{dq}T_{dq}^{m}C_{dr}A_{dr}^{m}C_{dq}A_{dr}TₖₛTₖₛAₖ₋THA₋GalNAc | 18 |

A subscript “d” indicates an unmodified, 2-β-D-deoxyribosyl sugar moiety.. A subscript “k” indicates a cEt. A superscript “m” indicates 5-methyl Cytosine. A subscript “s” indicates a stereorandom phosphorothioate internucleoside linkage, a subscript “r” indicates a phosphorothioate internucleoside linkage having the (Rp) configuration, and a subscript “q” indicates a phosphorothioate internucleoside linkage having the (Sp) configuration.

TABLE 127
In vitro toxicity and activity of modified oligonucleotides

| | in vitro | in vitro | |
|---|---|---|---|
| | CXCL12 | Caspase | |
| Compound | IC₅₀ | (% control) | % nucleolar |
| ID | (nM) | @ 20 μM | p54nrb |
| 855156 | 40 | 1437 | 90 |
| 1277251 | 130 | 174 | 6.4 |
| 1277252 | 111 | 525 | 62 |
| 1277253 | 111 | 138 | 12 |
| 1277254 | 20 | 336 | 24 |

### Example 60 Nucleosides with Two Chiral Phosphate Linkages in an Otherwise Stereorandom Phosphorthioate Nucleotide

Modified oligonucleotides containing chirally-controlled phosphorothioate linkages at two positions of the central region were synthesized. The compounds in the table below are 100% complementary to mouse CXCL12, GENBANK NT_039353.7 truncated from 69430515 to 69445350 (SEQ ID NO: 1), at position 6877 to 6892. Each compound with an ID in the range of 1273959-1273967 has a kkk-d(10)-kkk sugar motif, wherein each “k” represents a 2′-constrained ethyl modified sugar moiety and each “d” represents a 2′-deoxy sugar moiety. Each compound with an ID in the range of 1276491-1276497 has a kkk-d-m-d(8)-kkk sugar motif, wherein each “k” represents a cEt and each “d” represents a nucleoside comprising an unmodified, 2′-β-D-deoxyribosyl sugar moiety and each “m” represents nucleoside comprising a a 2′-Omethyl modified sugar moiety. Internucleoside linkages are as indicated in the table below, wherein a subscript “s” indicates a stereorandom phosphorothioate internucleoside linkage, a subscript “r” indicates a phosphorothioate internucleoside linkage having the (Rp) configuration and a subscript “q” indicates a phosphorothioate internucleoside linkage having the (Sp) configuration. Each compound contains an “Rp/Sp” unit comprising an internucleoside linkage having the (Rp) configuration followed by an internucleoside linkage having the (Sp) configuration, from 5′-3′.

Compounds were tested in 3T3-L1 cells for caspase activation as described in Example 1 above.

TABLE 128
Modified oligonucleotides

| | | SEQ |
|---|---|---|
| | | ID |
| Compound ID | Chemistry Notation | NO |
| 1273959 | Gₖₛ^{m}CₖₛAₖₛT_{dr}G_{dq}T_{ds}T_{ds}^{m}C_{ds}T_{ds}^{m}C_{ds}A_{ds}^{m}C_{ds}A_{ds}TₖₛTₖₛAₖ | 18 |
| 1273960 | Gₖₛ^{m}CₖₛAₖₛT_{ds}G_{dr}T_{dq}T_{ds}^{m}C_{ds}T_{ds}^{m}C_{ds}A_{ds}^{m}C_{ds}A_{ds}TₖₛTₖₛAₖ | 18 |
| 1273961 | Gₖₛ^{m}CₖₛAₖₛT_{ds}G_{ds}T_{dr}T_{dq}^{m}C_{ds}T_{ds}^{m}C_{ds}A_{ds}^{m}C_{ds}A_{ds}TₖₛTₖₛAₖ | 18 |
| 1273962 | Gₖₛ^{m}CₖₛAₖₛT_{ds}G_{ds}T_{ds}T_{dr}^{m}C_{dq}T_{ds}^{m}C_{ds}A_{ds}^{m}C_{ds}A_{ds}TₖₛTₖₛAₖ | 18 |
| 1273963 | Gₖₛ^{m}CₖₛAₖₛT_{ds}G_{ds}T_{ds}T_{ds}^{m}C_{dr}T_{dq}^{m}C_{ds}A_{ds}^{m}C_{ds}A_{ds}TₖₛTₖₛAₖ | 18 |
| 1273964 | Gₖₛ^{m}CₖₛAₖₛT_{ds}G_{ds}T_{ds}T_{ds}^{m}C_{ds}T_{dr}^{m}D_{dq}A_{ds}^{m}C_{ds}A_{ds}TₖₛTₖₛAₖ | 18 |
| 1273965 | Gₖₛ^{m}CₖₛAₖₛT_{ds}G_{ds}T_{ds}T_{ds}^{m}C_{ds}T_{ds}^{m}C_{dr}A_{dq}^{m}C_{ds}A_{ds}TₖₛTₖₛAₖ | 18 |
| 1273966 | Gₖₛ^{m}CₖₛAₖₛT_{ds}G_{ds}T_{ds}T_{ds}^{m}C_{ds}T_{ds}^{m}C_{ds}A_{dr}^{m}D_{dq}A_{ds}TₖₛTₖₛAₖ | 18 |
| 1273967 | Gₖₛ^{m}CₖₛAₖₛT_{ds}G_{ds}T_{ds}T_{ds}^{m}C_{ds}T_{ds}^{m}C_{ds}A_{ds}^{m}C_{dr}A_{dq}TₖₛTₖₛAₖ | 18 |
| 1276491 | Gₖₛ^{m}CₖₛAₖₛT_{ds}GₘₛT_{dr}T_{dq}^{m}C_{ds}T_{ds}^{m}C_{ds}A_{ds}^{m}C_{ds}A_{ds}TₖₛTₖₛAₖ | 18 |
| 1276492 | Gₖₛ^{m}CₖₛAₖₛT_{ds}GₘₛT_{ds}T_{dr}^{m}C_{dq}T_{ds}^{m}C_{ds}A_{ds}^{m}C_{ds}A_{ds}TₖₛTₖₛAₖ | 18 |
| 1276493 | Gₖₛ^{m}CₖₛAₖₛT_{ds}GₘₛT_{ds}T_{ds}^{m}C_{dr}T_{dq}^{m}C_{ds}A_{ds}^{m}C_{ds}A_{ds}TₖₛTₖₛAₖ | 18 |
| 1276494 | Gₖₛ^{m}CₖₛAₖₛT_{ds}GₘₛT_{ds}T_{ds}^{m}C_{ds}T_{dr}^{m}D_{dq}A_{ds}^{m}C_{ds}A_{ds}TₖₛTₖₛAₖ | 18 |
| 1276495 | Gₖₛ^{m}CₖₛAₖₛT_{ds}GₘₛT_{ds}T_{ds}^{m}C_{ds}T_{ds}^{m}C_{dr}A_{dq}^{m}C_{ds}A_{ds}TₖₛTₖₛAₖ | 18 |
| 1276496 | Gₖₛ^{m}CₖₛAₖₛT_{ds}GₘₛT_{ds}T_{ds}^{m}C_{ds}T_{ds}^{m}C_{ds}A_{dr}^{m}D_{dq}A_{ds}TₖₛTₖₛAₖ | 18 |
| 1276497 | Gₖₛ^{m}CₖₛAₖₛT_{ds}GₘₛT_{ds}T_{ds}^{m}C_{ds}T_{ds}^{m}C_{ds}A_{ds}^{m}C_{dr}A_{dq}TₖₛTₖₛAₖ | 18 |

A subscript “d” indicates an unmodified, 2′-β-D-deoxyribosyl sugar moiety. A subscript “k” indicates a cEt. A superscript “m” before a C indicates 5-methyl Cytosine. A subscript “s” indicates a stereorandom phosphorothioate internucleoside linkage, a subscript “r” indicates a phosphorothioate internucleoside linkage having the (Rp) configuration, and a subscript “q” indicates a phosphorothioate internucleoside linkage having the (Sp) configuration. A subscript “m” represents a 2′-Omethyl modified sugar moiety.

TABLE 129
Caspase activation in 3T3L1 cells

| | in vitro |
|---|---|
| | Caspase |
| Compound | (% control) |
| ID | @ 20 μM |
| 1273959 | 1138 |
| 1273960 | 654 |
| 1273961 | 1036 |
| 1273962 | 752 |
| 1273963 | 1349 |
| 1273964 | 907 |
| 1273965 | 984 |
| 1273966 | 750 |
| 1273967 | 785 |
| 1276491 | 116 |
| 1276492 | 450 |
| 1276493 | 234 |
| 1276494 | 85 |
| 1276495 | 214 |
| 1276496 | 165 |
| 1276497 | 148 |

### Example 61

Modified oligonucleotides were designed based on compounds 546108, 546118, 465178, and 694804, described in Examples 18, 18, 19, and 13, respectively. For sequences with a T at position 5 (from the 5′ end) in the parent kkk-d(10)-kkk oligonucleotide, the kkk-d-m-d(8)-kkk contains a 2′-OMe modified U at this position. For sequences with a mC at position 5 (from the 5′ end) in the parent kkk-d(10)-kkk oligonucleotide, the kkk-d-m-d(8)-kkk contains a 2′-OMe modified C at this position, lacking the 5-methyl group. Each of the modified oligonucleotides is conjugated with a THA-GalNAc conjugate group at the 3′-end. The oligonucleotides were evaluated for in vivo toxicity at a single dose after 72 hours.

Six week old BALB/C mice (purchased from Charles River) were injected subcutaneously once at 50 mg/kg with the modified oligonucleotides shown below or with saline control. Each treatment group consisted of 3 animals. The mice were sacrificed 72 hours following administration, and organs and plasma were harvested for further analysis.

TABLE 130
Modified Oligonucleotides

| | | position of | | | |
|---|---|---|---|---|---|
| | | altered | | | |
| | | nucleotide | modification | | SEQ |
| Compound | | in central | of altered | Chemistry Notation | ID |
| ID | Target | region | nucleotide | (5′ to 3′) | NO |
| 1306441 | HDAC | N/A | N/A | TₖₛAₖₛGₖₛT_{ds}^{m}C_{ds}T_{ds}^{m}C_{ds}T_{ds}G_{ds}T_{ds}^{m}C_{ds}A_{ds}G_{ds}Tₖ | 105 |
| | | | | TₖₛAₖ-THA-GalNAc | |
| 1306442 | HDAC | 2 | 2′-OMe | TₖₛAₖₛGₖₛT_{ds}CₘₛT_{ds}^{m}C_{ds}T_{ds}G_{ds}T_{ds}^{m}C_{ds}A_{ds}G_{ds}Tₖₛ | 105 |
| | | | | TₖₛAₖ-THA-GalNAc | |
| 1306443 | HDAC | 2 | MOP | TₖₛAₖₛGₖₛT_{ds}^{m}C_{dx}T_{ds}^{m}C_{ds}T_{ds}G_{ds}T_{ds}^{m}C_{ds}A_{ds}G_{ds}Tₖ | 105 |
| | | | | TₖₛAₖ-THA-GalNAc | |
| 1306444 | HDAC | 3 | MOP | TₖₛAₖₛGₖₛT_{ds}^{m}C_{ds}T_{dx}^{m}C_{ds}T_{ds}G_{ds}T_{ds}^{m}C_{ds}A_{ds}G_{ds}Tₖ | 105 |
| | | | | TₖₛAₖ-THA-GalNAc | |
| 1306445 | HDAC | 3 | MOP | Aₖₛ^{m}Cₖₛ^{m}Cₖₛ^{m}C_{ds}T_{ds}^{m}CdxA_{ds}A_{ds}G_{ds}T_{ds}^{m}C_{ds}T_{ds}^{m} | 109 |
| | | | | C_{ds}^{m}CₖₛTₖₛGₖ-THA-GalNAc | |
| 1306446 | FXI | N/A | N/A | GₖₛTₖₛ^{m}CₖₛA_{ds}G_{ds}T_{ds}A_{ds}T_{ds}^{m}C_{ds}^{m}C_{ds}^{m}C_{ds}A_{ds}G_{ds} | 89 |
| | | | | TₖₛGₖₛTₖ-THA-GalNAc | |
| 1306447 | FXI | 2 | 2′-OMe | GₖₛTₖₛ^{m}CₖₛA_{ds}GₘₛT_{ds}A_{ds}T_{ds}^{m}Cₖₛ^{m}C_{ds}^{m}C_{ds}A_{ds}G_{ds} | 89 |
| | | | | TₖₛGₖₛTₖ-THA-GalNAc | |
| 1306448 | FXI | 2 | MOP | GₖₛTₖₛ^{m}CₖₛA_{ds}G_{dx}T_{ds}A_{ds}T_{ds}^{m}C_{ds}^{m}C_{ds}^{m}C_{ds}A_{ds}G_{ds} | 89 |
| | | | | TₖₛGₖₛTₖ-THA-GalNAc | |
| 1306449 | FXI | 3 | MOP | GₖₛTₖₛ^{m}CₖₛA_{ds}G_{ds}T_{dx}A_{ds}T_{ds}^{m}C_{ds}^{m}C_{ds}^{m}C_{ds}A_{ds}G_{ds} | 89 |
| | | | | TₖₛGₖₛTₖ-THA-GalNAc | |
| 1306450 | DMN2 | N/A | N/A | AₖₛGₖₛAₖₛ^{m}C_{ds}T_{ds}^{m}C_{ds}T_{ds}^{m}C_{ds}G_{ds}G_{ds}T_{ds}T_{ds}^{m}C_{ds}^{m} | 49 |
| | | | | CₖₛGkSAK-THA-GalNAc | |
| 1306451 | DMN2 | 2 | 2′-OMe | AₖₛGₖₛAₖₛ^{m}C_{ds}Uₘₛ″C_{ds}T_{ds}^{m}C_{ds}G_{ds}G_{ds}T_{ds}T_{ds}^{m}C_{ds} | 127 |
| | | | | CₖₛGₖₛAₖ-THA-GalNAc | |
| 1306452 | DMN2 | 2 | MOP | AₖₛGₖₛAₖₛ^{m}C_{ds}T_{dx}^{m}C_{ds}T_{ds}^{m}C_{ds}G_{ds}G_{ds}T_{ds}T_{ds}^{m}C_{ds} | 49 |
| | | | | ^{m}CₖₛGₖₛAₖ-THA-GalNAc | |
| 1306453 | DMN2 | 3 | MOP | AₖₛGₖₛAₖₛ^{m}C_{ds}T_{ds}^{m}C_{dx}T_{ds}^{m}C_{ds}G_{ds}G_{ds}T_{ds}T_{ds}^{m}C_{ds} | 49 |
| | | | | ^{m}CₖₛGₖₛAₖ-THA-GalNAc | |

A subscript “d” indicates an unmodified, 2′-β-D-deoxyribosyl sugar moiety. A subscripts “k” indicates a cEt. A subscript “s” indicates a phosphorothioate internucleoside linkage. A superscript “m” before a C indicates 5-methyl Cytosine. A subscript “x” indicates a MOP internucleoside linkage.. A subscript “m” indicates a 2′-O-methyl-β-D-ribofuranosyl sugar moiety.

TABLE 131
In vivo Toxicity of Modified Oligonucleotides

| | position of | | |
|---|---|---|---|
| | altered | modification | |
| Compound | nucleotide in | of altered | ALT |
| ID | central region | nucleotide | (IU/L) |
| PBS | N/A | N/A | 28 |
| 1306441 | N/A | N/A | 2371 |
| 1306442 | 2 | 2′-OMe | 37 |
| 1306443 | 2 | MOP | 30 |
| 1306444 | 3 | MOP | 38 |
| 1306445 | 3 | MOP | 51 |
| 1306446 | N/A | N/A | 1555 |
| 1306447 | 2 | 2′-OMe | 53 |
| 1306448 | 2 | MOP | 43 |
| 1306449 | 3 | MOP | 43 |
| 1306450 | N/A | N/A | 1058 |
| 1306451 | 2 | 2′-OMe | 34 |
| 1306452 | 2 | MOP | 25 |
| 1306453 | 3 | MOP | 23 |

### Example 62 Effect of 2′-OMe Incorporation on Delayed Toxicity of Modified Oligonucleotides Complementary to HDAC2

Modified oligonucleotides were synthesized with 2′ modifications as indicated in the table below. The compounds have the sugar motif kkk-d(10)-kkk (“parent”) or kkk-d-m-d(8)-kkk, where “k” represents a cEt, “d” represents an unmodified, 2′-β-D-deoxyribosyl sugar moiety, and “m” represents 2′-OMe-β-D-ribofuranosyl sugar moiety. For sequences with a T at position 5 (from the 5′ end) in the parent kkk-d(10)-kkk oligonucleotide, the kkk-d-m-d(8)-kkk contains a 2′-OMe modified U at this position. For sequences with a ^{m}C at position 5 (from the 5′ end) in the parent kkk-d(10)-kkk oligonucleotide, the kkk-d-m-d(8)-kkk contains a 2′-OMe modified C at this position, lacking the 5-methyl group. The modified oligonucleotides in the table below have a mixed backbone motif soossssssssssos or soosossssssssos, wherein each “s” represents a phosphorothioate internucleoside linkage and each “o” represents a phosphodiester internucleoside linkage, as indicated by the chemistry notation in the table.

For the in vivo toxicity study in the table below, four female C57/Bl6 mice per group were administered 300 μg modified oligonucleotide by intracerebroventricular (ICV) injection. At 8 weeks post-injection, mice were evaluated according to 7 different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not. After all 7 criteria were evaluated, the FOB scores were summed for each mouse and averaged within each treatment group. The results are presented in the table below.

TABLE 132
In vivo Activity and Toxicity of Modified oligonucleotides complementary to HDAC2

| | | HDAC2 | | |
|---|---|---|---|---|
| | | mRNA (% | 8 | SEQ |
| Compound | | control) | weeK | ID |
| ID | Chemistry notation | Cortex | FOB | NO: |
| 1282276 | GₖₛAₖₒ^{m}CₖₒT_{ds}^{m}C_{ds}T_{ds}^{m}C_{ds}T_{ds}G_{ds}A_{ds}T_{ds}G_{ds}A_{ds}TₖₒAₖₛ^{m}Cₖ | 86 | 5 | 97 |
| 1282277 | GₖₛAₖₒ^{m}CₖₒT_{ds}CₘₛT_{ds}^{m}C_{ds}T_{ds}G_{ds}A_{ds}T_{ds}G_{ds}A_{ds}TₖₒAₖₛ^{m}Cₖ | n.d. | 7 | 97 |
| 1282278 | GₖₛAₖₒ^{m}CₖₒT_{ds}CₘₒT_{ds}^{m}C_{ds}T_{ds}G_{ds}A_{ds}T_{ds}G_{ds}A_{ds}TₖₒAₖₛ^{m}Cₖ | 72 | 6 | 97 |
| 1282280 | ^{m}CₖₛTₖₒAₖₒT_{ds}A_{ds}^{m}C_{ds}^{m}C_{ds}A_{ds}T_{ds}^{m}C_{ds}T_{ds}^{m}C_{ds}T_{ds}^{m}CₖₒAₖₛTₖ | 44 | 2 | 98 |
| 1282296 | ^{m}CₖₛTₖₒAₖₒT_{ds}Aₘₛ^{m}C_{ds}^{m}C_{ds}A_{ds}T_{ds}^{m}C_{ds}T_{ds}^{m}C_{ds}T_{ds}^{m}CₖₒAₖₛTₖ | n.d. | 7 | 98 |
| 1282622 | ^{m}CₖₛTₖₒAₖₒT_{ds}Aₘₒ^{m}C_{ds}^{m}C_{ds}A_{ds}T_{ds}^{m}C_{ds}T_{ds}^{m}C_{ds}T_{ds}^{m}CₖₒAₖₛTₖ | 68 | 0 | 98 |
| 1282281 | AₖₛTₖₒTₖₒA_{ds}T_{ds}A_{ds}T_{ds}G_{ds}G_{ds}^{m}C_{ds}A_{ds}A_{ds}^{m}C_{ds}Tₖₛ^{m}CₖₛAₖ | 70 | 4 | 101 |
| 1282627 | AₖₛTₖₒTₖₒA_{ds}UₘₛA_{ds}T_{ds}G_{ds}G_{ds}^{m}C_{ds}A_{ds}A_{ds}^{m}C_{ds}Tₖₛ^{m}CₖₛAₖ | 50 | 0 | 139 |
| 1282282 | GₖₛAₖₒ^{m}CₖₒT_{ds}A_{ds}A_{ds}T_{ds}A_{ds}T_{ds}G_{ds}^{m}C_{ds}A_{ds}G_{ds}TₖₒTₖₛTₖ | 107 | 0 | 102 |
| 1282628 | GₖₛAₖₒ^{m}CₖₒT_{ds}AₘₛA_{ds}T_{ds}A_{ds}T_{ds}G_{ds}^{m}C_{ds}A_{ds}G_{ds}TₖₒTₖₛTₖ | 86 | 0 | 102 |
| 1282283 | GₖₛTₖₛ^{m}CₖₒA_{ds}A_{ds}A_{ds}T_{ds}T_{ds}^{m}C_{ds}A_{ds}A_{ds}G_{ds}G_{ds}GₖₒTₖₛTₖ | n.d. | 7 | 103 |
| 1282629 | GₖₛTₖₛ^{m}CₖₒA_{ds}AₘₛA_{ds}T_{ds}T_{ds}^{m}C_{ds}A_{ds}A_{ds}G_{ds}G_{ds}GₖₒTₖₛTₖ | n.d. | 7 | 103 |
| 1282284 | ^{m}CₖₛAₖₒTₖₒA_{ds}A_{ds}A_{ds}G_{ds}^{m}C_{ds}A_{ds}T_{ds}G_{ds}G_{ds}T_{ds}GₖₒGₖₛAₖ | n.d. | 7 | 104 |
| 1282630 | ^{m}CₖₛAₖₒTₖₒA_{ds}AₘₛA_{ds}G_{ds}^{m}C_{ds}A_{ds}T_{ds}G_{ds}G_{ds}T_{ds}GₖₒGₖₛAₖ | n.d. | 7 | 104 |
| 1224264 | GₖₛTₖₒAₖₒ^{m}C_{ds}^{m}C_{ds}T_{ds}A_{ds}T_{ds}A_{ds}G_{ds}T_{ds}^{m}C_{ds}T_{ds}^{m}CₖₒTₖₛGₖ | n.d. | 7 | 106 |
| 1282631 | GₖₛTₖₒAₖₒ^{m}C_{ds}CₘₛT_{ds}A_{ds}T_{ds}A_{ds}G_{ds}T_{ds}^{m}C_{ds}T_{ds}^{m}CₖₒTₖₛGₖ | n.d. | 7 | 106 |
| 1282285 | TₖₛTₖₒGₖₒ^{m}C_{ds}^{m}C_{ds}A_{ds}A_{ds}T_{ds}A_{ds}T_{ds}^{m}C_{ds}A_{ds}^{m}C_{ds}^{m}CₖₒAₖₛTₖ | n.d | 7 | 94 |
| 1282632 | TₖₛTₖₒGₖₒ^{m}C_{ds}CₘₛA_{ds}A_{ds}T_{ds}A_{ds}T_{ds}^{m}C_{ds}A_{ds}^{m}C_{ds}^{m}CₖₒAₖₛTₖ | 59 | 0 | 94 |
| 1282623 | TₖₛTₖₒGₖₒ^{m}C_{ds}CₘₒA_{ds}A_{ds}T_{ds}A_{ds}T_{ds}^{m}C_{ds}A_{ds}^{m}C_{ds}^{m}CₖₒAₖₛTₖ | 88 | 0 | 94 |
| 1282286 | ^{m}CₖₛAₖₒAₖₒ^{m}C_{ds}T_{ds}G_{ds}A_{ds}A_{ds}^{m}C_{ds}^{m}C_{ds}A_{ds}^{m}C_{ds}^{m}C_{ds}^{m}CₖₒGₖₛTₖ | 68 | 3 | 95 |
| 1282633 | ^{m}CₖₛAₖₒAₖₒ^{m}C_{ds}UₘₛG_{ds}A_{ds}A_{ds}^{m}C_{ds}^{m}C_{ds}A_{ds}^{m}C_{ds}^{m}C_{ds}^{m}CₖₒGₖₛTₖ | 71 | 0 | 138 |
| 1282287 | Gₖₛ^{m}CₖₒAₖₒ^{m}C_{ds}A_{ds}A_{ds}T_{ds}A_{ds}T_{ds}^{m}C_{ds}A_{ds}T_{ds}T_{ds}AₖₒAₖₛ^{m}Cₖ | 74 | 0 | 96 |
| 1282634 | Gₖₛ^{m}CₖₒAₖₒ^{m}C_{ds}AₘₛA_{ds}T_{ds}A_{ds}T_{ds}^{m}C_{ds}A_{ds}T_{ds}T_{ds}AₖₒAₖₛ^{m}Cₖ | 70 | 0 | 96 |
| 1282288 | ^{m}CₖₛAₖₒTₖₒ^{m}C_{ds}A_{ds}T_{ds}^{m}C_{ds}T_{ds}A_{ds}T_{ds}A_{ds}^{m}C_{ds}^{m}C_{ds}AₖₒTₖₛ^{m}Cₖ | 16 | 2 | 99 |
| 1282298 | ^{m}CₖₛAₖₒTₖₒ^{m}C_{ds}AₘₛT_{ds}^{m}C_{ds}T_{ds}A_{ds}T_{ds}A_{ds}^{m}C_{ds}^{m}C_{ds}AₖₒTₖₛ^{m}Cₖ | 18 | 4 | 99 |
| 1282624 | ^{m}CₖₛAₖₒTₖₒ^{m}C_{ds}AₘₒT_{ds}^{m}C_{ds}T_{ds}A_{ds}T_{ds}A_{ds}^{m}C_{ds}^{m}C_{ds}AₖₒTₖₛ^{m}Cₖ | 70 | 0 | 99 |
| 1224263 | Aₖₛ^{m}CₖₒAₖₒ^{m}C_{ds}A_{ds}T_{ds}T_{ds}T_{ds}A_{ds}G_{ds}^{m}C_{ds}A_{ds}T_{ds}GₖₒAₖₛ^{m}Cₖ | n.d. | 2 | 100 |
| 1282635 | Aₖₛ^{m}CₖₒAₖₒ^{m}C_{ds}AₘₛT_{ds}T_{ds}T_{ds}A_{ds}G_{ds}^{m}C_{ds}A_{ds}T_{ds}GₖₒAₖₛ^{m}Cₖ | 61 | 0 | 100 |
| 1282289 | TₖₛAₖₒGₖₒT_{ds}^{m}C_{ds}T_{ds}^{m}C_{ds}T_{ds}G_{ds}T_{ds}^{m}C_{ds}A_{ds}G_{ds}TₖₒTₖₛAₖ | n.d. | 7 | 105 |
| 1282621 | TₖₛAₖₒGₖₒT_{ds}CₘₛT_{ds}^{m}C_{ds}T_{ds}G_{ds}T_{ds}^{m}C_{ds}A_{ds}G_{ds}TₖₒTₖₛAₖ | n.d. | 7 | 105 |
| 1282625 | TₖₛAₖₒGₖₒT_{ds}CₘₒT_{ds}^{m}C_{ds}T_{ds}G_{ds}T_{ds}^{m}C_{ds}A_{ds}G_{ds}TₖₒTₖₛAₖ | n.d | 6 | 105 |
| 1282290 | Tₖₛ^{m}CₖₒAₖₒT_{ds}G_{ds}T_{ds}A_{ds}^{m}C_{ds}^{m}C_{ds}T_{ds}A_{ds}T_{ds}A_{ds}GₖₒTₖₛ^{m}Cₖ | 22 | 6 | 107 |
| 1282300 | Tₖₛ^{m}CₖₒAₖₒT_{ds}GₘₛT_{ds}A_{ds}^{m}C_{ds}^{m}C_{ds}T_{ds}A_{ds}T_{ds}A_{ds}GₖₒTₖₛ^{m}Cₖ | 60 | 0 | 107 |
| 1282626 | Tₖₛ^{m}CₖₒAₖₒT_{ds}GₘₒT_{ds}A_{ds}^{m}C_{ds}^{m}C_{ds}T_{ds}A_{ds}T_{ds}A_{ds}GₖₒTₖₛ^{m}Cₖ | 107 | 0 | 107 |
| 1282291 | Tₖₛ^{m}CₖₒTₖₒT_{ds}A_{ds}A_{ds}T_{ds}T_{ds}T_{ds}^{m}C_{ds}A_{ds}T_{ds}G_{ds}TₖₒAₖₛ^{m}Cₖ | 64 | 0 | 108 |
| 1282636 | Tₖₛ^{m}CₖₒTₖₒT_{ds}AₘₛA_{ds}T_{ds}T_{ds}T_{ds}^{m}C_{ds}A_{ds}T_{ds}G_{ds}TₖₒAₖₛ^{m}Cₖ | 65 | 0 | 108 |
| 1282292 | Aₖₛ^{m}Cₖₒ^{m}Cₖₒ^{m}C_{ds}T_{ds}^{m}C_{ds}A_{ds}A_{ds}G_{ds}T_{ds}^{m}C_{ds}T_{ds}^{m}C_{ds}^{m}CₖₒTₖₛGₖ | n.d. | 6 | 109 |
| 1282637 | Aₖₛ^{m}Cₖₒ^{m}Cₖₒ^{m}C_{ds}Uₘₛ^{m}C_{ds}A_{ds}A_{ds}G_{ds}T_{ds}^{m}C_{ds}T_{ds}^{m}C_{ds}^{m}CₖₒIₖₛGₖ | n.d. | 7 | 109 |

A subscript “d” indicates an unmodified, 2′-β-D-deoxyribosyl sugar moiety. A subscript “e” indicates a 2′-MOE-β-D-ribofuranosyl sugar moiety. A subscript “m” indicates a 2′-O-methyl-β-D-ribofuranosyl sugar moiety. A subscript “k” indicates a cEt. A subscript “s” indicates a phosphorothioate internucleoside linkage. A superscript “m” before a C indicates 5-methyl Cytosine. A subscript “x” indicates a MOP internucleoside linkage.

### Example 63 Effect of Incorporation of 2′-OMe at Various Positions

Modified oligonucleotides were synthesized with 2′ modifications as indicated in the table below. The compounds have the sugar motif of kkk-d-m-d(8)-kkk, kkk-d(8)-m-d-kkk, or kkk-mm-d(8)-kkk where “k” represents a cEt, “d” represents an unmodified, 2′-β-D-deoxyribosyl sugar moiety, and “in” represents a a 2′-OMe-β-D-ribofuranosyl sugar moiety. For parent sequences with a T at the 2′-OMe-modified position, modified sequences contain a 2′-OMe modified U at this position. For parent sequences with a mC at the 2′-OMe-modified position, modified sequences contain a 2′-OMe modified C at this position, lacking the 5-methyl group.

TABLE 133
Modified Oligonucleotides

| | | position of | | | |
|---|---|---|---|---|---|
| | | altered | | | |
| | | nucleotide | modification | | SEQ |
| Compound | | in central | of altered | Chemistry Notation | ID |
| ID | Target | region | nucleotide | (5′ to 3′) | NO |
| 936053 | CXCL12 | 2 | 2′-OMe | Gₖₛ^{m}CₖₛAₖₛT_{ds}GₘₛT_{ds}T_{ds}^{m}C_{ds}T_{ds}^{m}C_{ds}A_{ds}^{m}C_{ds} | 18 |
| | | | | A_{ds}TₖₛTₖₛAₖ | |
| 1244114 | CXCL12 | 9 | 2′-OMe | Gₖₛ^{m}CₖₛAₖₛT_{ds}G_{ds}T_{ds}T_{ds}^{m}C_{ds}T_{ds}^{m}C_{ds}A_{ds}Cₘₛ | 18 |
| | | | | A_{ds}TₖₛTₖₛAₖ | |
| 1306760 | CXCL12 | 1, 2 | 2′-OMe | Gₖₛ^{m}CₖₛAₖₛUₘₛGₘₛT_{ds}T_{ds}^{m}C_{ds}T_{ds}^{m}C_{ds}A_{ds}^{m} | 19 |
| | | | | C_{ds}A_{ds}TₖₛTₖₛAₖ | |
| 895155 | SOD-1 | 2 | 2′-OMe | TₖₛGₖₛAₖₛG_{ds}GₘₛT_{ds}^{m}C_{ds}^{m}C_{ds}T_{ds}G_{ds}^{m}C_{ds}A_{ds} | 26 |
| | | | | ^{m}C_{ds}TₖₛGₖₛGₖ | |
| 1308544 | SOD-1 | 9 | 2′-OMe | TₖₛGₖₛAₖₛG_{ds}G_{ds}T_{ds}^{m}C_{ds}^{m}C_{ds}T_{ds}G_{ds}^{m}C_{ds}Aₘₛ | 26 |
| | | | | C_{ds}TₖₛGₖₛGₖ | |
| 1309002 | SOD-1 | 1, 2 | 2′-OMe | TₖₛGₖₛAₖₛGₘₛGₘₛT_{ds}^{m}C_{ds}^{m}C_{ds}T_{ds}G_{ds}^{m}C_{ds}A_{ds}^{m} | 26 |
| | | | | C_{ds}TₖₛGₖₛGₖ | |
| 1133122 | HDAC2 | 2 | 2′-OMe | TₖₛAₖₛGₖₛT_{ds}CₘₛT_{ds}^{m}C_{ds}T_{ds}G_{ds}T_{ds}^{m}C_{ds}A_{ds}G_{ds} | 105 |
| | | | | TₖₛTₖₛAₖ | |
| 1308545 | HDAC2 | 9 | 2′-OMe | TₖₛAₖₛGₖₛT_{ds}^{m}C_{ds}T_{ds}^{m}C_{ds}T_{ds}G_{ds}T_{ds}^{m}C_{ds}Aₘₛ | 105 |
| | | | | G_{ds}TₖₛTₖₛAₖ | |
| 1309073 | HDAC2 | 1, 2 | 2′-OMe | TₖₛAₖₛGₖₛUₘₛCₘₛT_{ds}^{m}C_{ds}T_{ds}G_{ds}T_{ds}^{m}C_{ds}A_{ds} | 145 |
| | | | | G_{ds}TₖₛTₖₛAₖ | |

A subscript “d” indicates an unmodified, 2′-β-D-deoxyribosyl sugar moiety. A subscript “e” indicates a 2′-MOE-β-D-ribofuranosyl sugar moiety. A subscript “m” indicates a 2′-O-methyl-β-D-ribofuranosyl sugar moiety. A subscript “k” indicates a cEt. A subscript “s” indicates a phosphorothioate internucleoside linkage. A superscript “m” before a C indicates 5-methyl Cytosine.

Six week old BALB/C mice (purchased from Charles River) were injected subcutaneously once at dosage 1.8, 5.5, 16.7, 50, or 150 mg/kg with the modified oligonucleotides shown below or with saline control. Each treatment group consisted of 3 animals. The mice were sacrificed 72 hours following administration, and organs and plasma were harvested for further analysis. The oligonucleotides were evaluated for reduction in target mRNA expression levels in vivo. P21 and Tnfrsf10b mRNA levels were also measured. The transaminase levels (ALT and AST) for each dose were also measured.

TABLE 134
In vivo Activity and Toxicity of Modified Oligonucleotides

| | position of | ALT | ALT | ED50 |
|---|---|---|---|---|
| | altered | (IU/L) | (IU/L) | (mg/kg) |
| Compound | nucleotide in | @ 50 | @150 | for |
| ID | central region | mg/kg | mg/kg | target |
| 936053 | 2 | 11 | 109 | 3.5 |
| 1244114 | 9 | 9092 | death | 1.0 |
| 1306760 | 1, 2 | 16 | 436 | 3.0 |
| 895155 | 2 | 29 | 110 | 11.0 |
| 1308544 | 9 | 2054 | 14507 | 27.7 |
| 1309002 | 1, 2 | 6 | 64 | 47.6 |
| 1133122 | 2 | 31 | 76 | 2.7 |
| 1308545 | 9 | 24695 | death | 0.9 |
| 1309073 | 1, 2 | 28 | 128 | 3.1 |

### Example 63 Effect of Incorporation of 2′-OMe in a Modified Oligonucleotide with 11 Nucleosides in the Central Region

Modified oligonucleotides were synthesized with 2′ modifications as indicated in the table below. The compounds have the sugar motif of kkk-d(11)-kkk, kkk-d-m-d(9)-kkk, or kkk-dd-m-d(8)-kkk, where “k” represents a cEt, “d” represents an unmodified, 2′-β-D-deoxyribosyl sugar moiety, and “in” represents a a 2′-OMe-β-D-ribofuranosyl modified sugar moiety. For parent sequences with a T at the 2′-OMe-modified position, modified sequences contain a 2′-OMe modified U at this position.

TABLE 135
Modified Oligonucleotides

| | | position of | | | |
|---|---|---|---|---|---|
| | | altered | modification | | SEQ |
| Compound | | nucleoside in | of altered | Chemistry Notation | ID |
| ID | Target | central region | nculeoside | (5′ to 3′) | NO |
| 1280764 | CXCL12 | N/A | 2′-OMe | Gₖₛ^{m}CₖₛAₖₛT_{ds}GₘₛT_{ds}T_{ds}^{m}C_{ds}T_{ds}^{m}C_{ds} | 125 |
| | | | | A_{ds}^{m}C_{ds}A_{ds}T_{ds}TₖₛAₖₛTₖ | |
| 1280763 | CXCL12 | 2 | 2′-OMe | Gₖₛ^{m}CₖₛAₖₛT_{ds}G_{ds}T_{ds}T_{ds}^{m}C_{ds}T_{ds}^{m}C_{ds}A_{ds} | 125 |
| | | | | CₘₛA_{ds}T_{ds}TₖₛAₖₛTₖ | |
| 1306440 | CXCL12 | 3 | 2′-OMe | Gₖₛ^{m}CₖₛAₖₛT_{ds}G_{ds}UₘₛT_{ds}^{m}C_{ds}T_{ds}^{m}C_{ds} | 146 |
| | | | | A_{ds}^{m}C_{ds}A_{ds}T_{ds}TₖₛAₖₛTₖ | |

A subscript “d” indicates an unmodified, 2′-β-D-deoxyribosyl sugar moiety. A subscript “e” indicates a 2′-MOE-β-D-ribofuranosyl sugar moiety. A subscript “m” indicates a 2′-O-methyl-β-D-ribofuranosyl sugar moiety. A subscript “k” indicates a cEt. A subscript “s” indicates a phosphorothioate internucleoside linkage. A superscript “m” before a C indicates 5-methyl Cytosine.

Six week old BALB/C mice (purchased from Charles River) were injected subcutaneously once at dosage 50 or 150 mg/kg with the modified oligonucleotides shown below or with saline control. Each treatment group consisted of 3 animals. The mice were sacrificed 72 hours following administration, and organs and plasma were harvested for further analysis. P21 and Tnfrsf10b mRNA levels were measured. The transaminase levels (ALT and AST) for each dose were also measured.

TABLE 136
In vivo Activity and Toxicity of Modified Oligonucleotides
with an 11-base central region

| | position of | ALT | ALT | P21 | Tnfrsf10b |
|---|---|---|---|---|---|
| | 2′-OMe | (IU/L) | (IU/L) | mRNA | mRNA |
| Compound | nucleotide in | @ 50 | @150 | @150 | @ 150 |
| ID | central region | mg/kg | mg/kg | mg/kg | mg/kg |
| 1280764 | N/A | death | death | death | death |
| 1280763 | 2 | 109 | 112 | 236 | 460 |
| 1306440 | 3 | 5109 | 7614 | 7022 | 13361 |

### Example 65 Effect of Incorporation of 2′-OMe in a Modified Oligonucleotide on Kidney Toxicity

Modified oligonucleotides were synthesized as indicated in the table below. The compounds have the sugar motif of kkk-d(10)-kkk or kkk-d-m-d(8)-kkk, where “k” represents a cEt, “d” represents an unmodified, 2′-β-D-deoxyribosyl sugar moiety, and “m” represents a 2′-OMe-β-D-ribofuranosyl sugar moiety.

TABLE 137
Modified Oligonucleotides

| | position of | | | |
|---|---|---|---|---|
| | altered | modification | | |
| Compound | nucleotide in | of altered | Chemistry Notation | SEQ |
| ID | central region | nucleotide | (5′ to 3′) | ID NO |
| 683702 | N/A | N/A | AₖₛCₖₛAₖₛA_{ds}G_{ds}G_{ds}A_{ds}^{m}C_{ds}A_{ds}^{m} | 147 |
| | | | C_{ds}^{m}C_{ds}A_{ds}A_{ds}GₖₛAₖₛTₖₛ | |
| 1295373 | 2 | 2′-OMe | AₖₛCₖₛAₖₛA_{ds}GₘₛG_{ds}A_{ds}^{m}C_{ds}A_{ds}^{m} | 147 |
| | | | C_{ds}^{m}C_{ds}A_{ds}A_{ds}GₖₛAₖₛTₖₛ | |

A subscript “d” indicates an unmodified, 2′-β-D-deoxyribosyl sugar moiety. A subscript “e” indicates a 2′-MOE-β-D-ribofuranosyl sugar moiety. A subscript “m” indicates a 2′-O-methyl-β-D-ribofuranosyl sugar moiety. A subscript “k” indicates a cEt. A subscript “s” indicates a phosphorothioate internucleoside linkage. A superscript “m” before a C indicates 5-methyl Cytosine.

8-10 week old Sprague Dawley rats were injected subcutaneously at dosage 50 mg/kg/week for two weeks (3 total injections) with the modified oligonucleotides shown below or with saline control. Each treatment group consisted of 3 animals. The mice were sacrificed 72 hours following administration, and organs and plasma were harvested for further analysis. KIM-1, NGAL, P21 and Tnfrsf10b mRNA levels were measured. Primer probe set rHAVCR1 (forward sequence: GGGATTACAGAGATCGTGACTGATT (SEQ ID NO: 148), reverse sequence: TGCAGCTGGAAGAACCAAAA (SEQ ID NO: 149), probe sequence CAGAGTAAAATACCCATTCCAGCTCCTGGG (SEQ ID NO: 150)) was used to measure KIM-1 and primer probe set RTS4389 (forward sequence: GATTCGTCAGCTTTGCCAAGT (SEQ ID NO: 151), reverse sequence: CGTCTGTTCAGTTGTCAATGCA (SEQ ID NO:152), probe sequence TCTGGGCCTCAAGGATAACAACATCGTTT (SEQ ID NO: 153)) was used to measure NGAL. The transaminase levels (ALT and AST) for each dose were also measured.

TABLE 138
In vivo Toxicity of Modified Oligonucleotides in kidney

| | 2′-OMe | | | | | |
|---|---|---|---|---|---|---|
| | position | | P21 | KIM-1 | NGAL | P21 |
| Compound | in central | ALT | mRNA | mRNA | mRNA | mRNA |
| ID | region | (IU/L) | (liver) | (kidney) | (kidney) | (kidney) |
| 683702 | N/A | 393 | 1243 | 3449 | 741 | 439 |
| 1295373 | 2 | 39 | 92 | 122 | 142 | 98 |

### Example 66 Nucleosides with Various Chemistries at Position 2 and 3 of the Central Region

Modified oligonucleotides containing altered nucleotides at position 2 of the central region were synthesized. The compounds in the table below are 100% complementary to mouse FXI. The sequence of the oligonucleotides is GTTATTGTGGTTGGCG (SEQ ID NO: 81), GTTAUTGTGGTTGGCG (SEQ ID NO: 133), or GTTATUGTGGTTGGCG (SEQ ID NO: 154) as indicated in the table below. The compounds have the sugar motif kkk-d-Z-d(8)-kkk or kkk-dd-Z-d(7)-kkk, where “k” represents a cEt, “d” represents an unmodified, 2′-β-D-deoxyribosyl sugar moiety, and “Z” represents a nucleotide comprising a modification as indicated in Table 139 below.

Compounds were tested in 3T3-L1 cells for caspase activation essentially as described in Example 1 above.

TABLE 139
Modified oligonucleotides and in vitro toxicity

| | position of | | | |
|---|---|---|---|---|
| | altered | | | in vitro |
| | nucleotide | Modification | | Caspase |
| | in central | of altered | SEQ | (% control) |
| Compound ID | region | nucleotide | ID NO | @ 40 μM |
| 464924 | N/A | N/A | 81 | 246 |
| 1326529 | 2 | cEt | 81 | 593 |
| 1326530 | 3 | cEt | 81 | 376 |
| 1326531 | 2 | 2′-MOE | 81 | 146 |
| 1326532 | 3 | 2′-MOE | 81 | 121 |
| 1133247 | 2 | 2′-OMe | 133 | 133 |
| 1326533 | 3 | 2′-OMe | 154 | 126 |
| 1326534 | 2 | 2′-FANA | 133 | 65 |
| 1326535 | 3 | 2′-FANA | 154 | 158 |
| 1326536 | 2 | 2′-ribo-F | 133 | 116 |
| 1326537 | 3 | 2′-ribo-F | 154 | 103 |
| 1326538 | 2 | F-HNA | 81 | 115 |
| 1326539 | 3 | F-HNA | 81 | 298 |
| 1351257 | 2 | LNA | 81 | 665 |
| 1351258 | 3 | LNA | 81 | 136 |
| 1351259 | 2 | α-L-LNA | 133 | 217 |
| 1351260 | 3 | α-L-LNA | 154 | 114 |
| 1351261 | 2 | ENA | 81 | 175 |
| 1351262 | 3 | ENA | 81 | 209 |

“cEt” has the meaning set forth herein.
“2′-MOE” has the meaning set forth herein.
“2′-OMe” has the meaning set forth herein.
“2′-FANA” indicates a 2′-F-β-D-arabinofuranosyl modified sugar moiety.
“F-HNA” has the meaning set forth herein.
“2′-ribo-F” indicates a 2′-fluororibose.
“LNA” has the meaning set forth herein.
“α-L-LNA” has the meaning set forth herein.
“ENA” has the meaning set forth herein.

### Example 67 Nucleosides with Various Chemistries at Position 2 and 3 of the Central Region

Modified oligonucleotides with 2′-5′ internucleoside linkages in the central region were synthesized as indicated in the table below, “k” represents a cEt, and “d” represents an unmodified, 2′-β-D-deoxyribosyl sugar moiety, and “25s” represents a 2′-5′ internucleoside linkage. An example of a 2′-5′ internucleoside linkage is shown below:

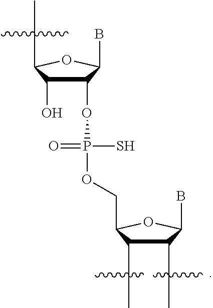

These modified oligonucleotides were compared to the otherwise identical modified oligonucleotide lacking an altered internucleoside linkage nucleotide in the central region, a 3-10-3 cEt gapmer, having three cEt nucleosides in each of the 5′ and 3′ regions and 10 DNA nucleosides in the central region (compound 558807). The modified oligonucleotides were also compared to a modified oligonucleotide having a 2′OMe at position 2 of the central region (Compound 936053). As demonstrated by the caspase activity, placement of a 2′-5′ internucleoside linkage at certain positions in the central region reduces caspase activity compared to the otherwise identical modified oligonucleotide lacking an altered internucleoside linkage. The compounds in the table below are 100% complementary to mouse CXCL12, GENBANK NT_039353.7 truncated from 69430515 to 69445350 (SEQ ID NO: 1), at position 6877 to 6892.

The compounds were tested in Hepa1-6 cells for caspase activation essentially as described in Example 1 above and the results are shown in the table below.

TABLE 140
Modified oligonucleotides complementary to CXCL12

| | position | | | |
|---|---|---|---|---|
| | of altered | | in vitro | |
| | linkage in | | Caspase | SEQ |
| Compound | central | | (% control) | ID |
| ID | region | Chemistry Notation (5′-3′) | @ 20 μM | NO |
| 558807 | n/a | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 3843 | 18 |
| 936053 | n/a | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{ds}TₖₛTₖₛAₖ | 148 | 18 |
| 1273969 | 1 | Gₖₛ^{m}CₖₛAₖₛU_{d25s}GₘₛT_{ds}T_{ds}^{m}C_{ds}T_{ds}^{m}C_{ds}A_{ds}^{m}C_{ds}A_{ds}TₖₛTₖₛAₖ | 538 | 19 |
| 1306771 | 2 | Gₖₛ^{m}CₖₛAₖₛT_{ds}Gₘ₂₅ₛT_{ds}T_{ds}^{m}C_{ds}T_{ds}^{m}C_{ds}A_{ds}^{m}C_{ds}A_{ds}TₖₛTₖₛAₖ | 526 | 18 |
| 1307546 | 3 | Gₖₛ^{m}CₖₛAₖₛT_{ds}GₘₛU_{d25s}T_{ds}^{m}C_{ds}T_{ds}^{m}C_{ds}A_{ds}^{m}C_{ds}A_{ds}TₖₛTₖₛAₖ | 164 | 20 |
| 1306773 | 4 | Gₖₛ^{m}CₖₛAₖₛT_{ds}GₘₛT_{ds}Uₐ₂₅ₛ^{m}C_{ds}T_{ds}^{m}C_{ds}A_{ds}^{m}C_{ds}A_{ds}TₖₛTₖₛAₖ | 210 | 21 |
| 1306777 | 5 | Gₖₛ^{m}CₖₛAₖₛT_{ds}GₘₛT_{ds}T_{ds}^{m}C_{d25s}T_{ds}^{m}C_{ds}A_{ds}^{m}C_{ds}A_{ds}TₖₛTₖₛAₖ | 4293 | 18 |
| 1309496 | 6 | Gₖₛ^{m}CₖₛAₖₛT_{ds}GₘₛT_{ds}T_{ds}^{m}C_{ds}U_{d25s}^{m}C_{ds}A_{ds}^{m}C_{ds}A_{ds}TₖₛTₖₛAₖ | 3744 | 39 |
| 1306759 | 7 | Gₖₛ^{m}CₖₛAₖₛT_{ds}GₘₛT_{ds}T_{ds}^{m}C_{ds}T_{ds}^{m}C_{d25s}A_{ds}^{m}C_{ds}A_{ds}TₖₛTₖₛAₖ | 3408 | 18 |
| 1306738 | 8 | Gₖₛ^{m}CₖₛAₖₛT_{ds}GₘₛT_{ds}T_{ds}^{m}C_{ds}T_{ds}^{m}C_{ds}A_{d25s}^{m}C_{ds}A_{ds}TₖₛTₖₛAₖ | 2162 | 18 |
| 1306931 | 9 | Gₖₛ^{m}CₖₛAₖₛT_{ds}GₘₛT_{ds}T_{ds}^{m}C_{ds}T_{ds}^{m}C_{ds}A_{ds}^{m}C_{d25s}A_{ds}TₖₛTₖₛAₖ | 4384 | 18 |
| 1306769 | 10 | Gₖₛ^{m}CₖₛAₖₛT_{ds}GₘₛT_{ds}T_{ds}^{m}C_{ds}T_{ds}^{m}C_{ds}A_{ds}^{m}C_{ds}A_{d25s}TₖₛTₖₛAₖ | 4769 | 18 |

1.-18. (canceled)

19. An oligomeric compound comprising a modified oligonucleotide consisting of 16 to 20 linked nucleosides, wherein the modified oligonucleotide has a 5′-region, a central region, and a 3′-region, wherein:
the 5′-region consists of 1-5 linked nucleosides, each comprising a modified sugar moiety;
the 3′ region consists of 1-5 linked nucleosides, each comprising a modified sugar moiety;
and the central region consists of 9-10 linked nucleosides, wherein
the 5′-most portion of the central region has the following formula: 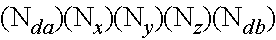
wherein one of Nₓ, N_{y}, and N_{z} is a safety-enhancing nucleoside;
the other two of Nₓ, N_{y}, and N_{z} are unmodified 2′-β-D-deoxyribosyl sugar moieties; and
N_{da} and N_{db} are unmodified 2′-β-D-deoxyribosyl sugar moieties.

20. The oligomeric compound of claim 19, wherein Nₓ is the safety-enhancing nucleoside.

21. The oligomeric compound of claim 20, wherein the safety-enhancing nucleoside has a sugar moiety selected from a morpholino, a cEt bicyclic sugar moiety, a LNA sugar moiety, an ENA sugar moiety, a 5′-methyl substituted 2′-deoxyribosyl sugar moiety, a 5′-ethyl substituted 2′-deoxyribosyl sugar moiety, a 5′-allyl substituted 2′-deoxyribosyl sugar moiety, and a 2′-β-L-deoxyxylosyl sugar moiety.

22. The oligomeric compound of claim 19, wherein N_{y} is the safety-enhancing nucleoside.

23. The oligomeric compound of claim 22, wherein the safety-enhancing nucleoside has a sugar moiety selected from a morpholino, a cEt bicyclic sugar moiety, a LNA sugar moiety, an ENA sugar moiety, a 5′-methyl substituted 2′-deoxyribosyl sugar moiety, a 5′-ethyl substituted 2′-deoxyribosyl sugar moiety, a 5′-allyl substituted 2′-deoxyribosyl sugar moiety, and a 2′-β-L-deoxyxylosyl sugar moiety.

24. The oligomeric compound of claim 23, wherein the safety-enhancing nucleoside is a 5′-methyl substituted 2′-deoxyribosyl sugar moiety, a 5′-ethyl substituted 2′-deoxyribosyl sugar moiety, or a 5′-allyl substituted 2′-deoxyribosyl sugar moiety.

25. The oligomeric compound of claim 19, wherein N_{z} is the safety-enhancing nucleoside.

26. The oligomeric compound of claim 25, wherein the safety-enhancing nucleoside has a sugar moiety selected from a morpholino, a cEt bicyclic sugar moiety, a LNA sugar moiety, an ENA sugar moiety, a 5′-methyl substituted 2′-deoxyribosyl sugar moiety, a 5′-ethyl substituted 2′-deoxyribosyl sugar moiety, a 5′-allyl substituted 2′-deoxyribosyl sugar moiety, and a 2′-β-L-deoxyxylosyl sugar moiety.

27. The oligomeric compound of claim 26, wherein the safety-enhancing nucleoside is a 5′-methyl substituted 2′-deoxyribosyl sugar moiety, a 5′-ethyl substituted 2′-deoxyribosyl sugar moiety, or a 5′-allyl substituted 2′-deoxyribosyl sugar moiety.

28. The oligomeric compound of claim 19, wherein each modified sugar moiety of the 3′ region and each modified sugar moiety of the 5′ region is independently selected from a 2′-substituted sugar moiety and a 4′-to-2′ linked bicyclic sugar moiety.

29. The oligomeric compound of claim 28, wherein each 4′-to-2′ linked bicyclic sugar moiety is independently selected from cEt, LNA, and ENA.

30. The oligomeric compound of claim 28, wherein each 4′-to-2′ linked bicyclic sugar moiety is cEt.

31. The oligomeric compound of claim 28, wherein each 2′-substituted sugar moiety has a 2′-substituent independently selected from 2′-MOE, 2′-OMe, and 2′-NMA.

32. The oligomeric compound of claim 28, wherein each 2′-substituted sugar moiety has a 2′-MOE substituent.

33. The oligomeric compound of claim 19, wherein the 5′ region consists of 3 nucleosides.

34. The oligomeric compound of claim 19, wherein the 3′ region consists of 3 nucleosides.

35. The oligomeric compound of claim 19, wherein each nucleobase of each nucleoside of the modified oligonucleotide is independently selected from thymine, uracil, guanine, cytosine, 5-methylcytosine, and adenine.

36. The oligomeric compound of claim 19, wherein each internucleoside linkage is independently selected from a phosphodiester internucleoside linkage and a phosphorothioate internucleoside linkage.

37. The oligomeric compound of claim 19, wherein the nucleobase sequence of the modified oligonucleotide is at least 85%, at least 90%, at least 95%, or 100% complementary to a target RNA.

38. The oligomeric compound of claim 37, wherein the target RNA is a target mRNA or a target pre-mRNA.
